(19) United States
(12) Patent Application Publication
(10) Pub. No.: US 2017/0291905 A1
(43) Pub. Date: Oct. 12, 2017

(54) ARGININE METHYLTRANSFERASE INHIBITORS AND USES THEREOF

(71) Applicant: Epizyme, Inc., Cambridge, MA (US)

(72) Inventors: Richard Chesworth, Concord, MA (US); Lorna Helen Mitchell, Cambridge, MA (US); John Emmerson Campbell, Cambridge, MA (US); Lawrence Alan Reiter, Mystic, CT (US); Kerren Kalai Swinger, Lexington, MA (US)

(73) Assignee: Epizyme, Inc., Cambridge, MA (US)

(21) Appl. No.: 15/511,509

(22) PCT Filed: Sep. 17, 2015
(86) PCT No.: PCT/US15/50750

§ 371 Date: Mar. 15, 2017

Related U.S. Application Data
(60) Provisional application No. 62/051,907, filed on Sep. 17, 2014.

(51) Int. Cl. C07D 493/08; C07D 417/04; C07D 403/04; C07D 405/14; C07D 231/12; C07D 405/04

(52) U.S. Cl. CPC C07D 493/08; C07D 231/12; C07D 405/04; C07D 403/04; C07D 405/14; C07D 417/04

(57) ABSTRACT

Described herein are compounds of Formula (S-I), pharmaceutically acceptable salts thereof, and pharmaceutical compositions thereof. Compounds described herein are useful for inhibiting arginine methyltransferase activity. Methods of using the compounds for treating arginine methyltransferase-mediated disorders are also described.

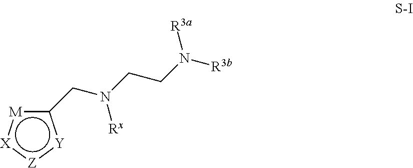

### RELATED APPLICATIONS

The present application claims priority under 35 U.S.C. §119(e) to U.S. provisional patent application, U.S. Ser. No. 62/051,907, filed Sep. 17, 2014, the entire contents of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., arginine methyltransferases), many of which are associated with specific genetic alterations that can cause human disease.

Disease-associated chromatin-modifying enzymes (e.g., arginine methyltransferases) play a role in diseases such as proliferative disorders, autoimmune disorders, muscular disorders, vascular disorders, metabolic disorders, and neurological disorders. Thus, there is a need for the development of small molecules that are capable of inhibiting the activity of arginine methyltransferases.

### DETAILED DESCRIPTION OF CERTAIN EMBODIMENTS

Arginine methyltransferases are attractive targets for modulation given their role in the regulation of diverse biological processes. It has now been found that compounds described herein, and pharmaceutically acceptable salts and compositions thereof, are effective as inhibitors of arginine methyltransferases. Such compounds have the general Formula (S-I):

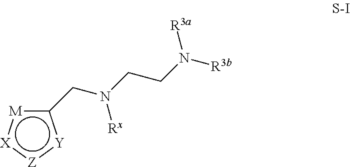

or a pharmaceutically acceptable salt thereof, wherein X, Y, Z, M, R^{3a}, R^{3b}, and R^{x} are as defined herein.

In some embodiments, pharmaceutical compositions are provided which comprise a compound described herein (e.g., a compound of Formula (S-I)), or a pharmaceutically acceptable salt thereof, and optionally a pharmaceutically acceptable excipient.

In certain embodiments, compounds described herein inhibit activity of an arginine methyltransferase (RMT) (e.g., PRMT1, PRMT3, CARM1, PRMT6, and/or PRMT8). In certain embodiments, methods of inhibiting an arginine methyltransferase are provided which comprise contacting the arginine methyltransferase with an effective amount of a compound of Formula (S-I), or a pharmaceutically acceptable salt thereof. The RMT may be purified or crude, and may be present in a cell, tissue, or a subject. Thus, such methods encompass inhibition of RMT activity both in vitro and in vivo. In certain embodiments, the RMT is wild-type. In certain embodiments, the RMT is overexpressed. In certain embodiments, the RMT is a mutant. In certain embodiments, the RMT is in a cell. In some embodiments, the RMT is expressed at normal levels in a subject, but the subject would benefit from RMT inhibition (e.g., because the subject has one or more mutations in an RMT substrate that causes an increase in methylation of the substrate with normal levels of RMT). In some embodiments, the RMT is in a subject known or identified as having abnormal RMT activity (e.g., overexpression).

In certain embodiments, methods of modulating gene expression in a cell are provided which comprise contacting a cell with an effective amount of a compound of Formula (S-I), or a pharmaceutically acceptable salt thereof, or a pharmaceutical composition thereof. In certain embodiments, the cell in culture in vitro. In certain embodiments, cell is in an animal, e.g., a human.

In certain embodiments, methods of modulating transcription in a cell are provided which comprise contacting a cell with an effective amount of a compound of Formula (S-I), or a pharmaceutically acceptable salt thereof, or a pharmaceutical composition thereof. In certain embodiments, the cell in culture in vitro. In certain embodiments, the cell is in an animal, e.g., a human.

In some embodiments, methods of treating an RMT-mediated disorder (e.g., a PRMT1-, PRMT3-, CARM1-, PRMT6-, or PRMT8-mediated disorder) are provided which comprise administering to a subject suffering from an RMT-mediated disorder an effective amount of a compound described herein (e.g., a compound of Formula (S-I)), or a pharmaceutically acceptable salt thereof, or a pharmaceutical composition thereof. In certain embodiments, the RMT-mediated disorder is a proliferative disorder. In certain embodiments, compounds described herein are useful for treating cancer. In certain embodiments, compounds described herein are useful for treating breast cancer, prostate cancer, lung cancer, colon cancer, bladder cancer, or leukemia. In certain embodiments, the RMT-mediated disorder is a muscular disorder. In certain embodiments, the RMT-mediated disorder is an autoimmune disorder. In certain embodiments, the RMT-mediated disorder is a neurological disorder. In certain embodiments, the RMT-mediated disorder is a vascular disorder. In certain embodiments, the RMT-mediated disorder is a metabolic disorder.

Compounds described herein are also useful for the study of arginine methyltransferases in biological and pathological phenomena, the study of intracellular signal transduction pathways mediated by arginine methyltransferases, and the comparative evaluation of new RMT inhibitors.

This application refers to various issued patent, published patent applications, journal articles, and other publications, all of which are incorporated herein by reference.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

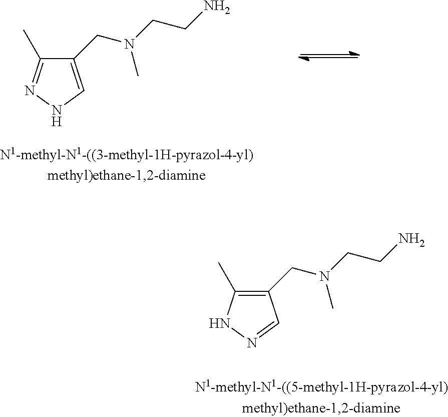

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Radical” refers to a point of attachment on a particular group. Radical includes divalent radicals of a particular group.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms and one or more carbon-carbon double bonds (e.g., 1, 2, 3, or 4 double bonds), and optionally one or more triple bonds (e.g., 1, 2, 3, or 4 triple bonds) (“C₂₋₂₀ alkenyl”). In certain embodiments, alkenyl does not comprise triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms and one or more carbon-carbon triple bonds (e.g., 1, 2, 3, or 4 triple bonds), and optionally one or more double bonds (e.g., 1, 2, 3, or 4 double bonds) (“C₂₋₂₀ alkynyl”). In certain embodiments, alkynyl does not comprise double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₄ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₄ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Fused” or “ortho-fused” are used interchangeably herein, and refer to two rings that have two atoms and one bond in common, e.g.,

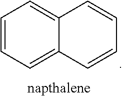

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

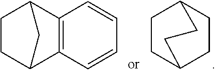

“Spiro” or “Spiro-fused” refers to a group of atoms which connect to the same atom of a carbocyclic or heterocyclic ring system (geminal attachment), thereby forming a ring, e.g.,

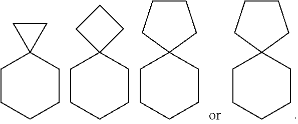

Spiro-fusion at a bridgehead atom is also contemplated.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₄ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₃₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In certain embodiments, heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, and thiiranyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary 5,6-bicyclic heteroaryl groups include, without limitation, any one of the following formulae:

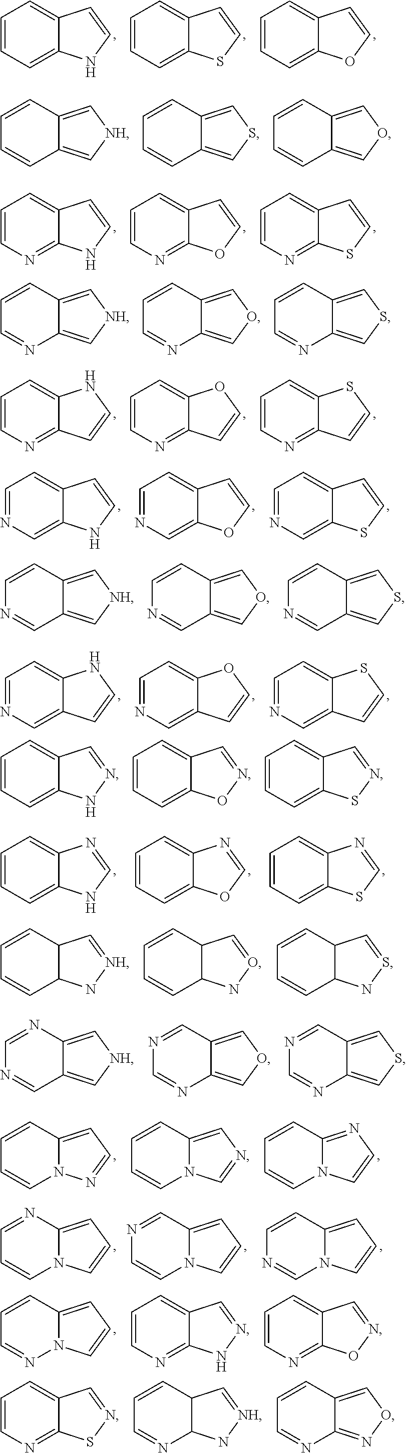

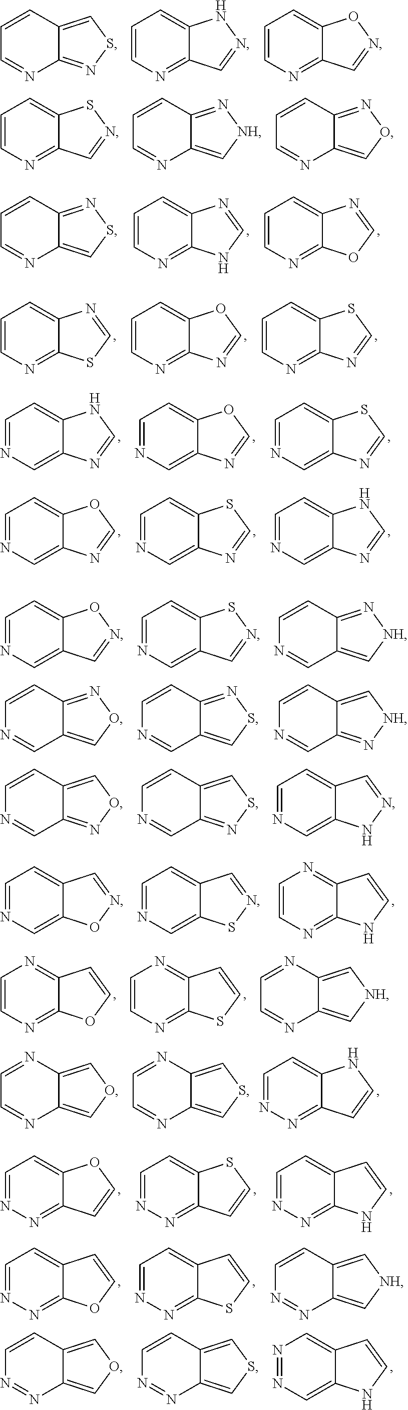

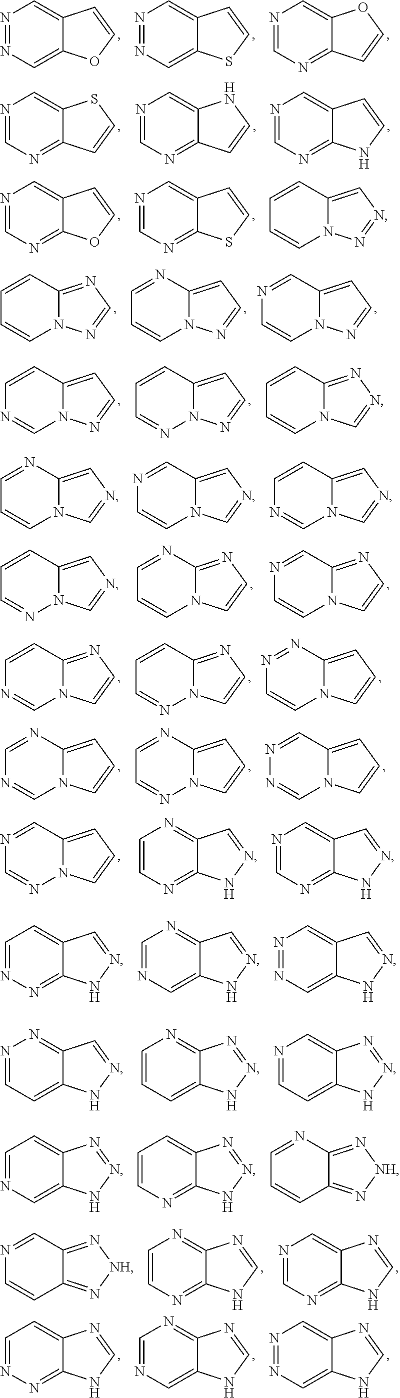

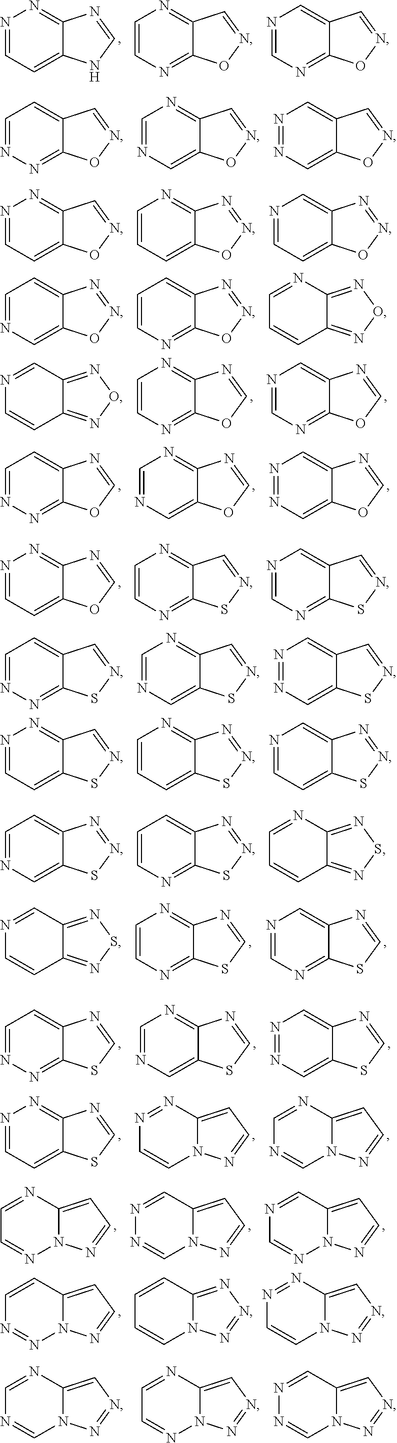

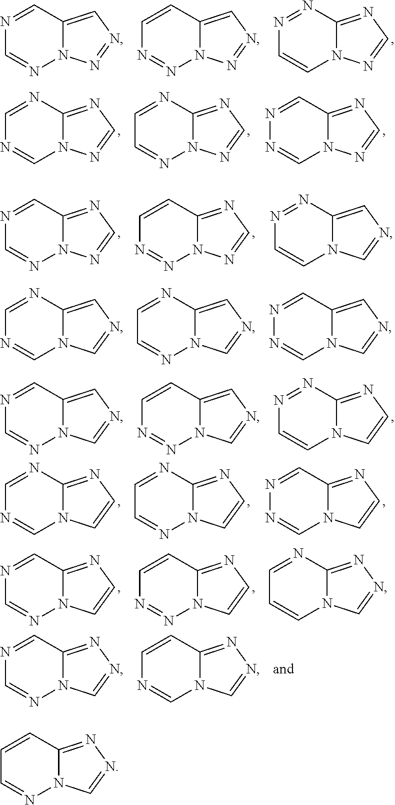

In any of the monocyclic or bicyclic heteroaryl groups, the point of attachment can be any carbon or nitrogen atom, as valency permits.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₄ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quanternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{c}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃10 carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

As generally described above, provided herein are compounds useful as arginine methyltransferase (RMT) inhibitors. In some embodiments, the present disclosure provides a compound of Formula (S-I):

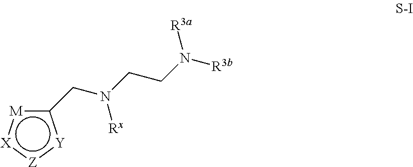

or a pharmaceutically acceptable salt thereof,
wherein

each of X, Y, and Z is independently O, S, N, NR⁴, or CR⁵, as valency permits;

R^{x} is optionally substituted C₁₋₄ alkyl or optionally substituted C₃₋₄ cycloalkyl;

M is —NR^{W1}— or —CR^{W2}—;

each of R^{W1} and R^{W2} is independently substituted cyclohexenyl, substituted cyclohexyl, or substituted tetrahydropyran;

each of R^{3a} and R^{3b} is independently hydrogen, optionally substituted C₁₋₄ alkyl, or optionally substituted C₃₋₄ cycloalkyl;

each instance of R⁴ is independently hydrogen or optionally substituted C₁₋₆ alkyl; and

each instance of R⁵ is independently hydrogen, halo, —CN, NO₂, optionally substituted C₁₋₄ alkyl, or optionally substituted C₃₋₄ cycloalkyl; and

provided that when M is —CR^{W2}—, at most one of X, Y, and Z is CR⁵;

provided that the compound is not one of the compounds in Table 1.

In certain embodiments, M is —NR^{W1}—, wherein R^{W1} is substituted cyclohexenyl, substituted cyclohexyl, or substituted tetrahydropyran. In certain embodiments, M is —CR^{W2}—, wherein R^{W2} is substituted cyclohexenyl, substituted cyclohexyl, or substituted tetrahydropyran.

In certain embodiments, when M is —CR^{W2}—, at most one of X, Y, and Z is CR⁵. In certain embodiments, when M is —CR^{W2}—, X is CR⁵. In certain embodiments, when M is —CR^{W2}—, Y is CR⁵. In certain embodiments, when M is —CR^{W2}—, Z is CR⁵. In certain embodiments, when M is —CR^{W2}—, each of X, Y, and Z is independently O, S, N, or NR⁴, as valency permits. In certain embodiments, when M is —CR^{W2}—, each of X, Y, and Z is independently O, N, or NR⁴, as valency permits.

In certain embodiments, a provided compound is of Formula (S-I-a):

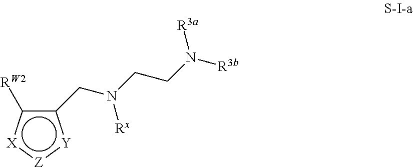

or a pharmaceutically acceptable salt thereof.

In certain embodiments, a provided compound is of Formula (S-I-b):

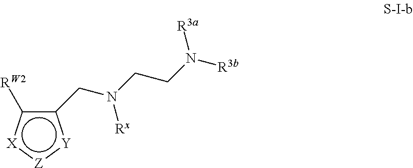

or a pharmaceutically acceptable salt thereof.

In certain embodiments, a provided compound is of Formula (S-II):

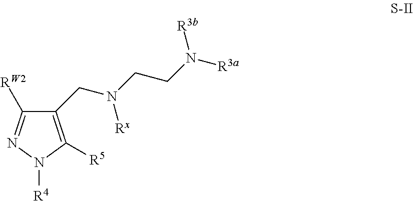

or a pharmaceutically acceptable salt thereof, wherein R^{W2}, R^{3a}, R^{3b}, R⁴, R⁵, and R^{x} are as described herein.

In certain embodiments, a provided compound is of Formula (S-III):

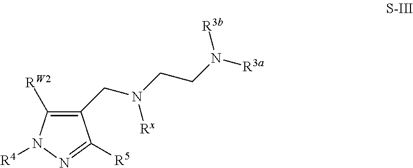

or a pharmaceutically acceptable salt thereof, wherein R^{W2}, R^{3a}, R^{3b}, R⁴, R⁵, and R^{x} are as described herein.

In certain embodiments, a provided compound is of Formula (S-IV):

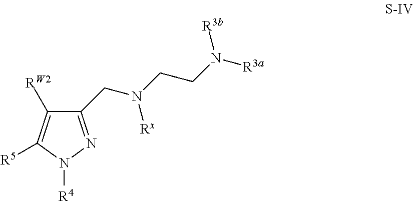

or a pharmaceutically acceptable salt thereof, wherein R^{W2}, R^{3a}, R^{3b}, R⁴, R⁵, and R^{x} are as described herein.

In certain embodiments, a provided compound is of Formula (S-V):

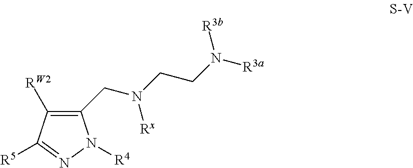

or a pharmaceutically acceptable salt thereof, wherein R^{W2}, R^{3a}, R^{3b}, R⁴, R⁵, and R^{x} are as described herein.

In certain embodiments, a provided compound is of Formula (S-VI):

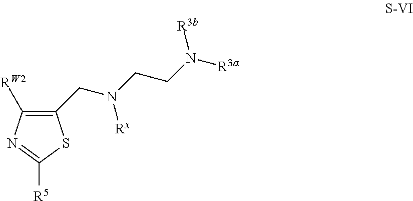

or a pharmaceutically acceptable salt thereof, wherein R^{W2}, R^{3a}, R^{3b}, R⁴, R⁵, and R^{x} are as described herein.

In certain embodiments, a provided compound is of Formula (S-VII):

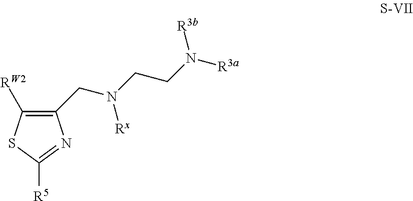

or a pharmaceutically acceptable salt thereof, wherein R^{W2}, R^{3a}, R^{3b}, R⁴, R⁵, and R^{x} are as described herein.

In certain embodiments, a provided compound is of Formula (S-VIII):

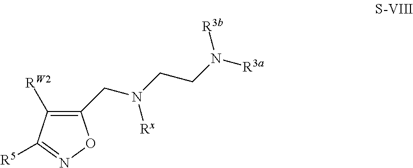

or a pharmaceutically acceptable salt thereof, wherein R^{W2}, R^{3a}, R^{3b}, R⁴, R⁵, and R^{x} are as described herein.

In certain embodiments, a provided compound is of Formula (S-IX):

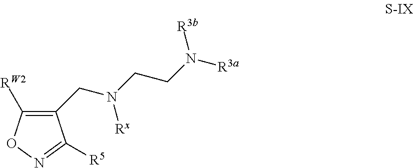

or a pharmaceutically acceptable salt thereof, wherein R^{W2}, R^{3a}, R^{3b}, R⁴, R⁵, and R^{x} are as described herein.

In certain embodiments, a provided compound is of Formula (S-X):

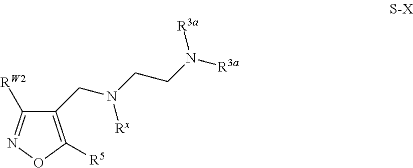

or a pharmaceutically acceptable salt thereof, wherein R^{W2}, R^{3a}, R^{3b}, R⁴, R⁵, and R^{x} are as described herein.

In certain embodiments, a provided compound is of Formula (S-XI):

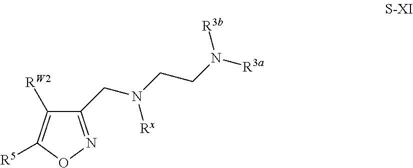

or a pharmaceutically acceptable salt thereof, wherein R^{W2}, R^{3a}, R^{3b}, R⁴, R⁵, and R^{x} are as described herein.

In certain embodiments, a provided compound is of Formula (S-XII):

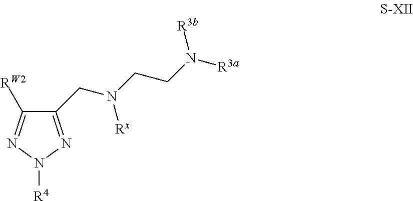

or a pharmaceutically acceptable salt thereof, wherein R^{W2}, R^{3a}, R^{3b}, R⁴, R⁵, and R^{x} are as described herein.

In certain embodiments, a provided compound is of Formula (S-XIII):

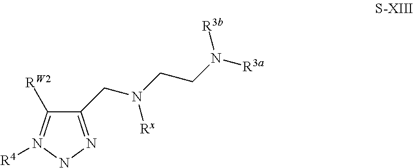

or a pharmaceutically acceptable salt thereof, wherein R^{W2}, R^{3a}, R^{3b}, R⁴, R⁵, and R^{x} are as described herein.

In certain embodiments, a provided compound is of Formula (S-XIV):

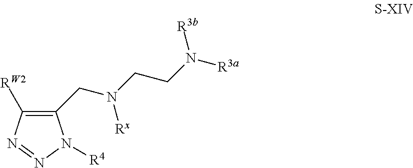

or a pharmaceutically acceptable salt thereof, wherein R^{W2}, R^{3a}, R^{3b}, R⁴, R⁵, and R^{x} are as described herein.

In certain embodiments, a provided compound is of Formula (S-XV):

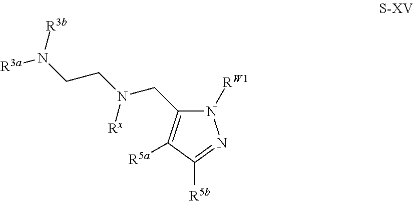

or a pharmaceutically acceptable salt thereof, wherein R^{W1}, R^{5a}, R^{5b}, R^{3a}, R^{3b}, R⁴, R⁵, and R^{x} are as described herein.

In certain embodiments, a provided compound is of Formula (S-XV):

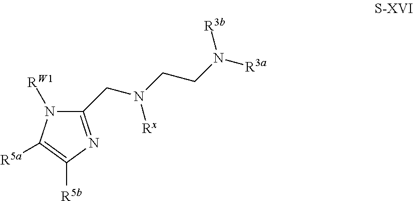

or a pharmaceutically acceptable salt thereof, wherein R^{W1}, R^{5a}, R^{5b}, R^{3a}, R^{3b}, R⁴, R⁵, and R^{x} are as described herein.

In certain embodiments, a provided compound is of Formula (S-XVII):

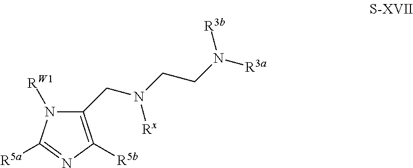

or a pharmaceutically acceptable salt thereof, wherein R^{W1}, R^{5a}, R^{5b}, R^{3a}, R^{3b}, R⁴, R⁵, and R^{x} are as described herein.

In certain embodiments, a provided compound is of Formula (S-XVIII):

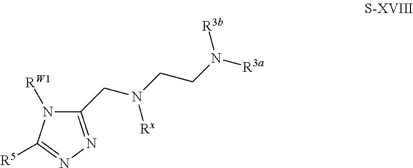

or a pharmaceutically acceptable salt thereof, wherein R^{W1}, R^{5a}, R^{5b}, R^{3a}, R^{3b}, R⁴, R⁵, and R^{x} are as described herein.

In certain embodiments, a provided compound is of Formula (S-XIX):

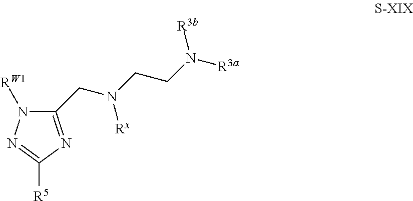

or a pharmaceutically acceptable salt thereof, wherein R^{W1}, R^{5a}, R^{5b}, R^{3a}, R^{3b}, R⁴, R⁵, and R^{x} are as described herein.

In certain embodiments, a provided compound is of Formula (S-XX):

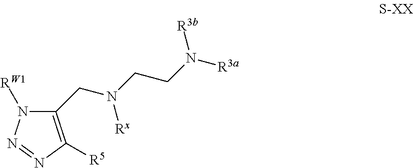

or a pharmaceutically acceptable salt thereof, wherein R^{W1}, R^{5a}, R^{5b}, R^{3a}, R^{3b}, R⁴, R⁵, and R^{x} are as described herein.

In certain embodiments of Formulae (S-I)-(S-XX), R^{x} is optionally substituted C₁₋₄ alkyl or optionally substituted C₃₋₄ cycloalkyl. In certain embodiments, R^{x} is optionally substituted C₁₋₄ alkyl. In certain embodiments, R^{x} is substituted C₁₋₄ alkyl. In certain embodiments, R^{x} is unsubstituted C₁₋₄ alkyl (e.g. methyl or ethyl). In certain embodiments, R^{x} is optionally substituted C₃₋₄ cycloalkyl (e.g. cyclopropyl).

In certain embodiments of Formulae (S-I)-(S-XX), R^{3a} is hydrogen, optionally substituted C₁₋₄ alkyl, or optionally substituted C₃₋₄ cycloalkyl. In certain embodiments, R^{3a} is hydrogen. In certain embodiments, R^{3a} is optionally substituted C₁₋₄ alkyl. In certain embodiments, R^{3a} is substituted C₁₋₄ alkyl. In certain embodiments, R^{3a} is unsubstituted C₁₋₄ alkyl (e.g. methyl or ethyl). In certain embodiments, R^{3a} is optionally substituted C₃₋₄ cycloalkyl (e.g. cyclopropyl).

In certain embodiments of Formulae (S-I)-(S-XX), R^{3b} is hydrogen, optionally substituted C₁₋₄ alkyl, or optionally substituted C₃₋₄ cycloalkyl. In certain embodiments, R^{3a} is hydrogen. In certain embodiments, R^{3b} is optionally substituted C₁₋₄ alkyl. In certain embodiments, R^{3b} is substituted C₁₋₄ alkyl. In certain embodiments, R^{3b} is unsubstituted C₁₋₄ alkyl (e.g. methyl or ethyl). In certain embodiments, R^{3b} is optionally substituted C₃₋₄ cycloalkyl (e.g. cyclopropyl).

In certain embodiments of Formulae (S-I)-(S-XX), R^{3a} and R^{3b} are both hydrogen. In certain embodiments of Formulae (S-I)-(S-XX), R^{3a} is hydrogen and R^{3b} is optionally substituted C₁₋₄ alkyl. In certain embodiments of Formulae (S-I)-(S-XX), R^{3a} is hydrogen and R^{3b} is unsubstituted C₁₋₄ alkyl (e.g. methyl). In certain embodiments of Formulae (S-I)-(S-XX), each of R^{3a} and R^{3b} is independently optionally substituted C₁₋₄ alkyl. In certain embodiments of Formulae (S-I)-(S-XX), each of R^{3a} and R^{3b} is independently unsubstituted C₁₋₄ alkyl. In certain embodiments of Formulae (S-I)-(S-XX), R^{3a} and R^{3b} are both methyl.

In certain embodiments of Formulae (S-I)-(S-XX), R⁴ is hydrogen or optionally substituted C₁₋₆ alkyl. In certain embodiments, R⁴ is hydrogen. In certain embodiments, R⁴ is optionally substituted C₁₋₆ alkyl. In certain embodiments, R⁴ is substituted C₁₋₆ alkyl. In certain embodiments, R⁴ is unsubstituted C₁₋₆ alkyl (e.g. methyl or ethyl).

In certain embodiments of Formulae (S-I)-(S-XX), each instance of R⁵ is independently hydrogen, halo, —CN, NO₂, optionally substituted C₁₋₄ alkyl, or optionally substituted C₃₋₄ cycloalkyl. In certain embodiments, R⁵ is independently hydrogen. In certain embodiments, R⁵ is halo, —CN, NO₂, optionally substituted C₁₋₄ alkyl, or optionally substituted C₃₋₄ cycloalkyl. In certain embodiments, R⁵ is halo (e.g. F, Cl, Br, or I). In certain embodiments, R⁵ is Cl. In certain embodiments, R⁵ is CN. In certain embodiments, R⁵ is NO₂. In certain embodiments, R⁵ is optionally substituted C₁₋₄ alkyl. In certain embodiments, R⁵ is substituted C₁₋₄ alkyl. In certain embodiments, R⁵ is unsubstituted C₁₋₄ alkyl (e.g. methyl or ethyl).

As generally defined herein, each instance of R^{5a} is independently hydrogen, halo, —CN, NO₂, or optionally substituted C₁₋₆ alkyl. In certain embodiments, R^{5a} is hydrogen. In certain embodiments, R^{5a} is halo (e.g. F, Cl, Br, or I). In certain embodiments, R^{5a} is Cl. In certain embodiments, R^{5a} is CN. In certain embodiments, R^{5a} is NO₂. In certain embodiments, R^{5a} is optionally substituted C₁₋₄ alkyl. In certain embodiments, R^{5a} is unsubstituted C₁₋₄ alkyl. In certain embodiments, R^{5a} is methyl or ethyl.

As generally defined herein, each instance of R^{5b} is independently hydrogen, halo, —CN, NO₂, or optionally substituted C₁₋₆ alkyl. In certain embodiments, R^{5b} is hydrogen. In certain embodiments, R^{5b} is halo (e.g. F, Cl, Br, or I). In certain embodiments, R^{5b} is Cl. In certain embodiments, R^{5b} is CN. In certain embodiments, R^{5b} is NO₂. In certain embodiments, R^{5b} is optionally substituted C₁₋₄ alkyl. In certain embodiments, R^{5b} is unsubstituted C₁₋₄ alkyl. In certain embodiments, R^{5b} is methyl or ethyl.

In certain embodiments, R^{5a} and R^{5b} are hydrogen. In certain embodiments, R^{5a} is hydrogen and R^{5b} is halo, —CN, NO₂, or optionally substituted C₁₋₆ alkyl. In certain embodiments, R^{5a} is hydrogen and R^{5b} is halo (e.g. Cl) or optionally substituted C₁₋₆ alkyl (e.g. methyl). In certain embodiments, R^{5b} is hydrogen and R^{5a} is halo (e.g. Cl) or optionally substituted C₁₋₆ alkyl (e.g. methyl). In certain embodiments, R^{5a} is halo (e.g. Cl) and R^{5b} is halo, —CN, NO₂, or optionally substituted C₁₋₆ alkyl. In certain embodiments, R^{5b} is halo (e.g. Cl) and R^{5a} is halo, —CN, NO₂, or optionally substituted C₁₋₆ alkyl. In certain embodiments, R^{5a} and R^{5b} are both halo (e.g. Cl). In certain embodiments, R^{5a} and R^{5b} are both unsubstituted C₁₋₆ alkyl (e.g. methyl).

In certain embodiments of Formulae (S-I)-(S-XX), R^{W2} is independently substituted cyclohexenyl, substituted cyclohexyl, or substituted tetrahydropyran. In some embodiments, R^{W2} is substituted cyclohexenyl. In certain embodiments, R^{W2} is substituted cyclohexyl. In certain embodiments, R^{W2} is substituted tetrahydropyran.

In certain embodiments, R^{W2} is substituted cyclohexyl of Formula (S-i):

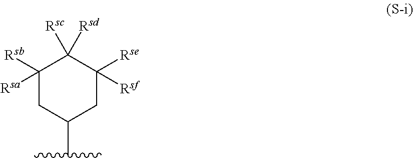

wherein

each of R^{sa}, R^{sb}, R^{se}, and R^{sf} is independently hydrogen, optionally substituted C₁₋₆ alkyl, —OR^{SO}, or —C(═O)N(R^{SN1})₂;

each of R^{sc} and R^{sd} is independently optionally substituted C₁₋₆ alkyl, —OR^{SO}, —C(═O)N(R^{SN1})₂, or —N(R^{SN2})₂;

each instance of R^{SN1} and R^{SN2} is independently hydrogen or optionally substituted alkyl;

each instance of R^{SO} is optionally substituted alkyl, optionally substituted alkenyl, optionally substituted alkynyl, optionally substituted heterocyclyl, optionally substituted carbocyclyl, optionally substituted aryl, optionally substituted heteroaryl; and

R^{sa} and R^{sb} are optionally taken together with the intervening atom to form an optionally substituted carbocylic ring or an optionally substituted heterocyclic ring;

R^{sc} and R^{sd} are optionally taken together with the intervening atom to form an optionally substituted carbocylic ring or an optionally substituted heterocyclic ring; and

R^{se} and R^{sf} are optionally taken together with the intervening atom to form an optionally substituted carbocylic ring or an optionally substituted heterocyclic ring.

In certain embodiments, R^{W2} is one of the following formulae:

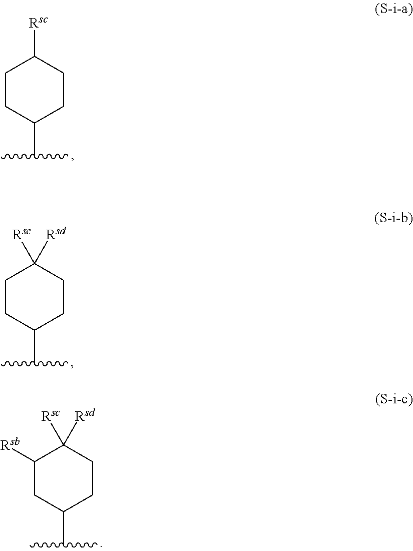

As generally defined herein, R^{sa} is independently hydrogen, optionally substituted C₁₋₆ alkyl, —OR^{SO}, or —C(═O)N(R^{SN1})₂. In certain embodiments, R^{sa} is hydrogen. In certain embodiments, R^{sa} is optionally substituted C₁₋₆ alkyl, —OR^{SO}, or —C(═O)N(R^{SN1})₂. In certain embodiments, R^{sa} is optionally substituted C₁₋₆ alkyl. In certain embodiments, R^{sa} is unsubstituted C₁₋₆ alkyl (e.g. methyl, ethyl, n-propyl, or iso-propyl). In certain embodiments, R^{sa} is substituted C₁₋₆ alkyl. In certain embodiments, R^{sa} is —CH₂—O—(CH₂)ₛₙ—X^{sc} or —CH₂—O—(CH₂)ₛₘ—O—X^{sd}; wherein sn is 0, 1, 2, 3, 4, 5, or 6; sm is 1, 2, 3, 4, 5, or 6; X^{sc} is hydrogen, optionally substituted C₁₋₆ alkyl, optionally substituted aryl, optionally substituted carbocyclyl, or optionally substituted heterocyclyl; and X^{sd} is optionally substituted C₁₋₆ alkyl, optionally substituted aryl, optionally substituted carbocyclyl, or optionally substituted heterocyclyl. In certain embodiments, R^{sa} is —CH₂—O—(CH₂)ₛₙ—X^{sc}, wherein X^{sc} is hydrogen or optionally substituted C₁₋₆ alkyl. In certain embodiments, R^{sa} is —CH₂—O—X^{sc}, wherein X^{sc} is hydrogen or optionally substituted C₁₋₆ alkyl. In certain embodiments, X^{sc} is unsubstituted C₁₋₆ alkyl (e.g. methyl or ethyl). In certain embodiments, X^{sc} is substituted C₁₋₆ alkyl. In certain embodiments, X^{sc} is C₁₋₆ haloalkyl (e.g. CF₃ or CH₂CF₃). In certain embodiments, R^{sa} is —CH₂—O—CH₂CF₃. In certain embodiments, R^{sa} is —CH₂—O—(CH₂)ₛₘ—O—X^{sd}, wherein X^{sd} is hydrogen or optionally substituted C₁₋₆ alkyl. In certain embodiments, X^{sd} is unsubstituted C₁₋₆ alkyl (e.g. methyl or ethyl). In certain embodiments, X^{sd} is substituted C₁₋₆ alkyl (e.g. CF₃ or CH₂CF₃). In certain embodiments, R^{sa} is —OR^{SO}, wherein R^{SO} is as defined herein. In certain embodiments, R^{sa} is —OR^{SO}, wherein R^{SO} is optionally substituted alkyl or optionally substituted heterocylyl. In certain embodiments, R^{SO} is optionally substituted alkyl. In certain embodiments, R^{SO} is unsubstituted alkyl (e.g. methyl, ethyl, n-propyl, or iso-propyl). In certain embodiments, R^{SO} is substituted alkyl (e.g. CF₃ or CH₂CF₃). In certain embodiments, R^{SO} is optionally substituted heterocylyl. In certain embodiments, R^{SO} is optionally substituted six-membered heterocylyl. In certain embodiments, R^{SO} is optionally substituted six-membered heterocyclyl with one heteroatom selected from the group consisting of N, S, and O. In certain embodiments, R^{SO} is optionally substituted tetrahydropyran. In certain embodiments, R^{sa} is —C(═O)N(R^{SN1})₂, wherein each instance of R^{SN1} is independently hydrogen or optionally substituted alkyl. In certain embodiments, R^{sa} is —C(═O)NHR^{SN1}, wherein R^{SN1} is independently hydrogen or optionally substituted alkyl. In certain embodiments, R^{SN1} is unsubstituted alkyl (e.g. methyl or ethyl).

As generally defined herein, R^{sb} is independently hydrogen, optionally substituted C₁₋₆ alkyl, —OR^{SO}, or —C(═O)N(R^{SN1})₂. In certain embodiments, R^{sb} is hydrogen. In certain embodiments, R^{sb} is optionally substituted C₁₋₆ alkyl, —OR^{SO}, or —C(═O)N(R^{SN1})₂. In certain embodiments, R^{sb} is optionally substituted C₁₋₆ alkyl. In certain embodiments, R^{sb} is unsubstituted C₁₋₆ alkyl (e.g. methyl, ethyl, n-propyl, or iso-propyl). In certain embodiments, R^{sb} is substituted C₁₋₆ alkyl. In certain embodiments, R^{sb} is —CH₂—O—(CH₂)ₛₙ—X^{sc} or —CH₂—O—(CH₂)ₛₘ—O—X^{sd}; wherein sn is 0, 1, 2, 3, 4, 5, or 6; sm is 1, 2, 3, 4, 5, or 6; X^{sc} is hydrogen, optionally substituted C₁₋₆ alkyl, optionally substituted aryl, optionally substituted carbocyclyl, or optionally substituted heterocyclyl; and X^{sd} is optionally substituted C₁₋₆ alkyl, optionally substituted aryl, optionally substituted carbocyclyl, or optionally substituted heterocyclyl. In certain embodiments, R^{sb} is —CH₂—O—(CH₂)ₛₙ—X^{sc}, wherein X^{sc} is hydrogen or optionally substituted C₁₋₆ alkyl. In certain embodiments, R^{sb} is —CH₂—O—X^{sc}, wherein X^{sc} is hydrogen or optionally substituted C₁₋₆ alkyl. In certain embodiments, X^{sc} is unsubstituted C₁₋₆ alkyl (e.g. methyl or ethyl). In certain embodiments, X^{sc} is substituted C₁₋₆ alkyl. In certain embodiments, X^{sc} is C₁₋₆ haloalkyl (e.g. CF₃ or CH₂CF₃). In certain embodiments, R^{sb} is —CH₂—O—CH₂CF₃. In certain embodiments, R^{sb}—CH₂—O—(CH₂)ₛₘ—O—X^{sd}, wherein X^{sd} is hydrogen or optionally substituted C₁₋₆ alkyl. In certain embodiments, X^{sd} is unsubstituted C₁₋₆ alkyl (e.g. methyl or ethyl). In certain embodiments, X^{sd} is substituted C₁₋₆ alkyl (e.g. CF₃ or CH₂CF₃). In certain embodiments, R^{sb} is —OR^{SO}, wherein R^{SO} is as defined herein. In certain embodiments, R^{sb} is —OR^{SO}, wherein R^{SO} is optionally substituted alkyl or optionally substituted heterocyclyl. In certain embodiments, R^{SO} is optionally substituted alkyl. In certain embodiments, R^{SO} is unsubstituted alkyl (e.g. methyl, ethyl, n-propyl, or iso-propyl). In certain embodiments, R^{SO} is substituted alkyl (e.g. CF₃ or CH₂CF₃). In certain embodiments, R^{SO} is optionally substituted heterocyclyl. In certain embodiments, R^{SO} is optionally substituted six-membered heterocyclyl. In certain embodiments, R^{SO} is optionally substituted six-membered heterocyclyl with one heteroatom selected from the group consisting of N, S, and O. In certain embodiments, R^{SO} is optionally substituted tetrahydropyran. In certain embodiments, R^{sb} is —C(═O)N(R^{SN1})₂, wherein each instance of R^{SN1} is independently hydrogen or optionally substituted alkyl. In certain embodiments, R^{sb} is —C(═O)NHR^{SN1}, wherein R^{SN1} is independently hydrogen or optionally substituted alkyl. In certain embodiments, R^{SN1} is unsubstituted alkyl (e.g. methyl or ethyl).

As generally defined herein, R^{se} is independently hydrogen, optionally substituted C₁₋₆ alkyl, —OR^{SO}, or —C(═O)N(R^{SN1})₂. In certain embodiments, R^{se} is hydrogen. In certain embodiments, R^{se} is optionally substituted C₁₋₆ alkyl, —OR^{SO}, or —C(═O)N(R^{SN1})₂. In certain embodiments, R^{se} is optionally substituted C₁₋₆ alkyl. In certain embodiments, R^{se} is unsubstituted C₁₋₆ alkyl (e.g. methyl, ethyl, n-propyl, or iso-propyl). In certain embodiments, R^{se} is substituted C₁₋₆ alkyl. In certain embodiments, R^{se} is —CH₂—O—(CH₂)ₛₙ—X^{sc} or —CH₂—O—(CH₂)ₛₘ—O—X^{sd}; wherein sn is 0, 1, 2, 3, 4, 5, or 6; sm is 1, 2, 3, 4, 5, or 6; X^{sc} is hydrogen, optionally substituted C₁₋₆ alkyl, optionally substituted aryl, optionally substituted carbocyclyl, or optionally substituted heterocyclyl; and X^{sd} is optionally substituted C₁₋₆ alkyl, optionally substituted aryl, optionally substituted carbocyclyl, or optionally substituted heterocyclyl. In certain embodiments, R^{se} is —CH₂—O—(CH₂)ₛₙ—X^{sc}, wherein X^{sc} is hydrogen or optionally substituted C₁₋₆ alkyl. In certain embodiments, R^{se} is —CH₂—O—X^{sc}, wherein X^{sc} is hydrogen or optionally substituted C₁₋₆ alkyl. In certain embodiments, X^{sc} is unsubstituted C₁₋₆ alkyl (e.g. methyl or ethyl). In certain embodiments, X^{sc} is substituted C₁₋₆ alkyl. In certain embodiments, X^{sc} is C₁₋₆haloalkyl (e.g. CF₃ or CH₂CF₃). In certain embodiments, R^{se} is —CH₂—O—CH₂CF₃. In certain embodiments, R^{se}—CH₂—O—(CH₂)ₛₘ—O—X^{sd}, wherein X^{sd} is hydrogen or optionally substituted C₁₋₆ alkyl. In certain embodiments, X^{sd} is unsubstituted C₁₋₆ alkyl (e.g. methyl or ethyl). In certain embodiments, X^{sd} is substituted C₁₋₆ alkyl (e.g. CF₃ or CH₂CF₃). In certain embodiments, R^{se} is —OR^{SO}, wherein R^{SO} is as defined herein. In certain embodiments, R^{se} is —OR^{SO}, wherein R^{SO} is optionally substituted alkyl or optionally substituted heterocyclyl. In certain embodiments, R^{SO} is optionally substituted alkyl. In certain embodiments, R^{SO} is unsubstituted alkyl (e.g. methyl, ethyl, n-propyl, or iso-propyl). In certain embodiments, R^{SO} is substituted alkyl (e.g. CF₃ or CH₂CF₃). In certain embodiments, R^{SO} is optionally substituted heterocyclyl. In certain embodiments, R^{SO} is optionally substituted six-membered heterocyclyl. In certain embodiments, R^{SO} is optionally substituted six-membered heterocyclyl with one heteroatom selected from the group consisting of N, S, and O. In certain embodiments, R^{SO} is optionally substituted tetrahydropyran. In certain embodiments, R^{se} is —C(═O)N(R^{SN1})₂, wherein each instance of R^{SN1} is independently hydrogen or optionally substituted alkyl. In certain embodiments, R^{se} is —C(═O)NHR^{SN1}, wherein R^{SN1} is independently hydrogen or optionally substituted alkyl. In certain embodiments, R^{SN1} is unsubstituted alkyl (e.g. methyl or ethyl).

As generally defined herein, R^{sf} is independently hydrogen, optionally substituted C₁₋₆ alkyl, —OR^{SO}, or —C(═O)N(R^{SN1})₂. In certain embodiments, R^{sf} is hydrogen. In certain embodiments, R^{sf} is optionally substituted C₁₋₆ alkyl, —OR^{SO}, or —C(═O)N(R^{SN1})₂. In certain embodiments, R^{sf} is optionally substituted C₁₋₆ alkyl. In certain embodiments, R^{sf} is unsubstituted C₁₋₆ alkyl (e.g. methyl, ethyl, n-propyl, or iso-propyl). In certain embodiments, R^{sf} is substituted C₁₋₆ alkyl. In certain embodiments, R^{sf} is —CH₂—O—(CH₂)ₛₙ—X^{sc} or —CH₂—O—(CH₂)ₛₘ—O—X^{sd}; wherein sn is 0, 1, 2, 3, 4, 5, or 6; sm is 1, 2, 3, 4, 5, or 6; X^{sc} is hydrogen, optionally substituted C₁₋₆ alkyl, optionally substituted aryl, optionally substituted carbocyclyl, or optionally substituted heterocyclyl; and X^{sd} is optionally substituted C₁₋₆ alkyl, optionally substituted aryl, optionally substituted carbocyclyl, or optionally substituted heterocyclyl. In certain embodiments, R^{sf} is —CH₂—O—(CH₂)ₛₙ—X^{sc} wherein X^{sc} is hydrogen or optionally substituted C₁₋₆ alkyl. In certain embodiments, R^{sf} is —CH₂—O—X^{sc}, wherein X^{sc} is hydrogen or optionally substituted C₁₋₆ alkyl. In certain embodiments, X^{sc} is unsubstituted C₁₋₆ alkyl (e.g. methyl or ethyl). In certain embodiments, X^{sc} is substituted C₁₋₆ alkyl. In certain embodiments, X^{sc} is C₁₋₆haloalkyl (e.g. CF₃ or CH₂CF₃). In certain embodiments, R^{sf} is —CH₂—O—CH₂CF₃. In certain embodiments, X^{sc} is optionally substituted heterocyclcyl. In certain embodiments, X^{sc} is optionally substituted six-membered heterocyclcyl. In certain embodiments, X^{sc} is unsubstituted tetrahydropyran. In certain embodiments, X^{sc} is

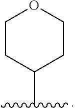 [SMILES: *C1CCOCC1]

In certain embodiments, X^{sc} is

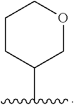 [SMILES: *C1CCCOC1]

In certain embodiments, X^{sc} is

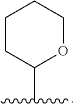 [SMILES: *C1CCCCO1]

In certain embodiments, R^{sf} is —CH₂—O—(CH₂)ₛₘ—O—X^{sd}, wherein X^{sd} is hydrogen or optionally substituted C₁₋₆ alkyl. In certain embodiments, X^{sd} is unsubstituted C₁₋₆ alkyl (e.g. methyl or ethyl). In certain embodiments, X^{sd} is substituted C₁₋₆ alkyl (e.g. CF₃ or CH₂CF₃). In certain embodiments, R^{sf} is —OR^{SO}, wherein R^{SO} is as defined herein. In certain embodiments, R^{sf} is —OR^{SO}, wherein R^{SO} is optionally substituted alkyl or optionally substituted heterocyclyl. In certain embodiments, R^{SO} is optionally substituted alkyl. In certain embodiments, R^{SO} is unsubstituted alkyl (e.g. methyl, ethyl, n-propyl, or iso-propyl). In certain embodiments, R^{SO} is substituted alkyl (e.g. CF₃ or CH₂CF₃). In certain embodiments, R^{SO} is optionally substituted heterocyclyl. In certain embodiments, R^{SO} is optionally substituted six-membered heterocyclyl. In certain embodiments, R^{SO} is optionally substituted six-membered heterocyclyl with one heteroatom selected from the group consisting of N, S, and O. In certain embodiments, R^{SO} is optionally substituted tetrahydropyran. In certain embodiments, R^{sf} is —C(═O)N(R^{SN1})₂, wherein each instance of R^{SN} is independently hydrogen or optionally substituted alkyl. In certain embodiments, R^{sf} is —C(═O)NHR^{SN1}, wherein R^{SN1} is independently hydrogen or optionally substituted alkyl. In certain embodiments, R^{SN1} is unsubstituted alkyl (e.g. methyl or ethyl).

As generally defined herein, R^{sc} is optionally substituted C₁₋₆ alkyl, —OR^{SO}, —C(═O)N(R^{SN1})₂, or —N(R^{SN2})₂, wherein R^{SN1} and R^{SN2} are as defined herein. In certain embodiments, R^{sc} is optionally substituted C₁₋₆ alkyl. In certain embodiments, R^{sc} is unsubstituted C₁₋₆ alkyl (e.g. methyl, ethyl, n-propyl, or iso-propyl). In certain embodiments, R^{sc} is substituted C₁₋₆ alkyl. In certain embodiments, R^{sc} is —CH₂—O—(CH₂)ₛₙ—X^{sc} or —CH₂—O—(CH₂)ₛₘ—O—X^{sd}; wherein sn is 0, 1, 2, 3, 4, 5, or 6; sm is 1, 2, 3, 4, 5, or 6; X^{sc} is hydrogen, optionally substituted C₁₋₆ alkyl, optionally substituted aryl, optionally substituted carbocyclyl, or optionally substituted heterocyclyl; and X^{sd} is optionally substituted C₁₋₆ alkyl, optionally substituted aryl, optionally substituted carbocyclyl, or optionally substituted heterocyclyl. In certain embodiments, R^{sc} is —CH₂—O—(CH₂)ₛₙ—X^{sc}, wherein X^{sc} is hydrogen or optionally substituted C₁₋₆ alkyl. In certain embodiments, R^{sc} is —CH₂—O—X^{sc}, wherein X^{sc} is hydrogen or optionally substituted C₁₋₆ alkyl. In certain embodiments, X^{sc} is unsubstituted C₁₋₆ alkyl (e.g. methyl or ethyl). In certain embodiments, X^{sc} is substituted C₁₋₆ alkyl. In certain embodiments, X^{sc} is C₁₋₆haloalkyl. (e.g. CF₃ or CH₂CF₃). In certain embodiments, R^{sc} is —CH₂—O—CH₂CF₃. In certain embodiments, R^{sc} is —CH₂—O—(CH₂)ₛₙ—X^{sc}, wherein X^{sc} is optionally substituted heterocyclcyl. In certain embodiments, X^{sc} is optionally substituted six-membered heterocyclcyl. In certain embodiments, X^{sc} is unsubstituted tetrahydropyran. In certain embodiments, X^{sc} is

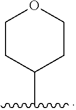 [SMILES: *C1CCOCC1]

In certain embodiments, X^{sc} is

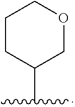 [SMILES: *C1CCCOC1]

In certain embodiments, X^{sc} is

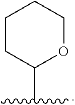 [SMILES: *C1CCCCO1]

In certain embodiments, R^{sc} is —CH₂—O—(CH₂)ₛₘ—O—X^{sd}, wherein X^{sd} is hydrogen or optionally substituted C₁₋₆ alkyl. In certain embodiments, X^{sd} is unsubstituted C₁₋₆ alkyl (e.g. methyl or ethyl). In certain embodiments, X^{sd} is substituted C₁₋₆ alkyl (e.g. CF₃ or CH₂CF₃). In certain embodiments, R^{sc} is —OR^{SO}, wherein R^{SO} is as defined herein. In certain embodiments, R^{sc} is —OR^{SO}, wherein R^{SO} is optionally substituted alkyl or optionally substituted heterocyclyl. In certain embodiments, R^{SO} is optionally substituted alkyl. In certain embodiments, R^{SO} is unsubstituted alkyl (e.g. methyl, ethyl, n-propyl, or iso-propyl). In certain embodiments, R^{SO} is substituted alkyl (e.g. CF₃ or CH₂CF₃). In certain embodiments, R^{SO} is optionally substituted heterocyclyl. In certain embodiments, R^{SO} is optionally substituted six-membered heterocyclyl. In certain embodiments, R^{SO} is optionally substituted six-membered heterocyclyl with one heteroatom selected from the group consisting of N, S, and O. In certain embodiments, R^{SO} is optionally substituted tetrahydropyran. In certain embodiments, R^{sc} is —C(═O)N(R^{SN1})₂, wherein each instance of R^{SN1} is independently hydrogen or optionally substituted alkyl. In certain embodiments, R^{sc} is —C(═O)NHR^{SN1}, wherein R^{SN1} is independently hydrogen or optionally substituted alkyl. In certain embodiments, R^{SN1} is unsubstituted alkyl (e.g. methyl or ethyl). In certain embodiments, R^{sc} is —N(R^{SN2})₂, wherein each instance of R^{SN2} is independently hydrogen or optionally substituted alkyl. In certain embodiments, R^{sc} is —NHR^{SN2}, wherein R^{SN2} is hydrogen or optionally substituted alkyl. In certain embodiments, R^{sc} is —NH₂. In certain embodiments, R^{sc} is —NHR^{SN2}, wherein R^{SN2} is optionally substituted alkyl. In certain embodiments, R^{SN2} is unsubstituted alkyl (e.g. methyl or ethyl).

As generally defined herein, R^{sd} is optionally substituted C₁₋₆ alkyl, —OR^{SO}, —C(═O)N(R^{SN1})₂, or —N(R^{SN2})₂, wherein R^{SN1} and R^{SN2} are as defined herein. In certain embodiments, R^{sd} is optionally substituted C₁₋₆ alkyl. In certain embodiments, R^{sd} is unsubstituted C₁₋₆ alkyl (e.g. methyl, ethyl, n-propyl, or iso-propyl). In certain embodiments, R^{sd} is substituted C₁₋₆ alkyl. In certain embodiments, R^{sd} is —CH₂—O—(CH₂)ₛₙ—X^{sc} or —CH₂—O—(CH₂)ₛₘ—O—X^{sd}; wherein sn is 0, 1, 2, 3, 4, 5, or 6; sm is 1, 2, 3, 4, 5, or 6; X^{sc} is hydrogen, optionally substituted C₁₋₆ alkyl, optionally substituted aryl, optionally substituted carbocyclyl, or optionally substituted heterocyclyl; and X^{sd} is optionally substituted C₁₋₆ alkyl, optionally substituted aryl, optionally substituted carbocyclyl, or optionally substituted heterocyclyl. In certain embodiments, R^{sd} is —CH₂—O—(CH₂)ₛₙ—X^{sc}, wherein X^{sc} is hydrogen or optionally substituted C₁₋₆ alkyl. In certain embodiments, R^{sd} is —CH₂—O—X^{sc}, wherein X^{sc} is hydrogen or optionally substituted C₁₋₆ alkyl. In certain embodiments, X^{sc} is unsubstituted C₁₋₆ alkyl (e.g. methyl or ethyl). In certain embodiments, X^{sc} is substituted C₁₋₆ alkyl. In certain embodiments, X^{sc} is C₁₋₆ haloalkyl. (e.g. CF₃ or CH₂CF₃). In certain embodiments, R^{sd} is —CH₂—O—CH₂CF₃. In certain embodiments, R^{sd} is —CH₂—O—(CH₂)ₛₘ—O—X^{sd}, wherein X^{sd} is hydrogen or optionally substituted C₁₋₆ alkyl. In certain embodiments, X^{sd} is unsubstituted C₁₋₆ alkyl (e.g. methyl or ethyl). In certain embodiments, X^{sd} is substituted C₁₋₆ alkyl (e.g. CF₃ or CH₂CF₃). In certain embodiments, R^{sd} is —OR^{SO}, wherein R^{SO} is as defined herein. In certain embodiments, R^{sd} is —OR^{SO}, wherein R^{SO} is optionally substituted alkyl or optionally substituted heterocyclyl. In certain embodiments, R^{SO} is optionally substituted alkyl. In certain embodiments, R^{SO} is unsubstituted alkyl (e.g. methyl, ethyl, n-propyl, or iso-propyl). In certain embodiments, R^{SO} is substituted alkyl (e.g. CF₃ or CH₂CF₃). In certain embodiments, R^{SO} is optionally substituted heterocyclyl. In certain embodiments, R^{SO} is optionally substituted six-membered heterocyclyl. In certain embodiments, R^{SO} is optionally substituted six-membered heterocyclyl with one heteroatom selected from the group consisting of N, S, and O. In certain embodiments, R^{SO} is optionally substituted tetrahydropyran. In certain embodiments, R^{sd} is —C(═O)N(R^{SN1})₂, wherein each instance of R^{SN1} is independently hydrogen or optionally substituted alkyl. In certain embodiments, R^{sd} is —C(═O)NHR^{SN1}, wherein R^{SN1} is independently hydrogen or optionally substituted alkyl. In certain embodiments, R^{SN1} is unsubstituted alkyl (e.g. methyl or ethyl). In certain embodiments, R^{sd} is —N(R^{SN2})₂, wherein each instance of R^{SN2} is independently hydrogen or optionally substituted alkyl. In certain embodiments, R^{sd} is —NHR^{SN2}, wherein R^{SN2} is hydrogen or optionally substituted alkyl. In certain embodiments, R^{sd} is —NH₂. In certain embodiments, R^{sd} is —NHR^{SN2}, wherein R^{SN2} is optionally substituted alkyl. In certain embodiments, R^{SN2} is unsubstituted alkyl (e.g. methyl or ethyl).

As generally defined herein, sn is 0, 1, 2, 3, 4, 5, or 6. In certain embodiments, sn is 0. In certain embodiments, sn is 1. In certain embodiments, sn is 2. In certain embodiments, sn is 3. In certain embodiments, sn is 4. In certain embodiments, sn is 5. In certain embodiments, sn is 6.

As generally defined herein, sm is 1, 2, 3, 4, 5, or 6. In certain embodiments, sm is 1. In certain embodiments, sm is 2. In certain embodiments, sm is 3. In certain embodiments, sm is 4. In certain embodiments, sm is 5. In certain embodiments, sm is 6.

In certain embodiments, R^{sc} and R^{sd} are the same. In certain embodiments, R^{sc} and R^{sd} are different.

In certain embodiments of Formulae (S-i) and (S-i-a)-(S-i-c), each instance of R^{sc} and R^{sd} is independently unsubstituted C₁₋₆ alkyl (e.g. methyl, ethyl, or n-propyl). In certain embodiments, R^{sc} and R^{sd} are both methyl. In certain embodiments of Formulae (S-i) and (S-i-a)-(S-i-c), each instance of R^{sc} and R^{sd} is independently substituted C₁₋₆ alkyl. In certain embodiments, each instance of R^{sc} and R^{sd} is independently —C₁₋₆alkyl-OH. In certain embodiments, R^{sc} and R^{sd} are —CH₂—OH. In certain embodiments, each instance of R^{sc} and R^{sd} is independently —CH₂—O—(CH₂)ₛₙ—X^{sc}, wherein X^{sc} is as defined herein. In certain embodiments, each instance of R^{sc} and R^{sd} is independently —CH₂—O—(CH₂)ₛₙ—X^{sc}, wherein X^{sc} is optionally substituted C₁₋₆ alkyl or optionally substituted heterocyclyl. In certain embodiments, X^{sc} is unsubstituted C₁₋₆ alkyl (e.g. methyl, ethyl, or n-propyl). In certain embodiments, X^{sc} is substituted C₁₋₆ alkyl (e.g. C₁₋₆ haloalkyl). In certain embodiments, X^{sc} is optionally substituted heterocyclcyl. In certain embodiments, X^{sc} is optionally substituted six-membered heterocyclcyl. In certain embodiments, X^{sc} is unsubstituted tetrahydropyran. In certain embodiments, X^{sc} is

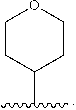 [SMILES: *C1CCOCC1]

In certain embodiments, X^{sc} is

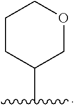 [SMILES: *C1CCCOC1]

In certain embodiments, X^{sc} is

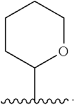 [SMILES: *C1CCCCO1]

In certain embodiments, each instance of R^{sc} and R^{sd} is independently —CH₂—O—(CH₂)ₛₙ—X^{se}, wherein X^{sc} is optionally substituted C₃₋₆ carbocyclyl (e.g. cyclopropyl). In certain embodiments, each instance of R^{sc} and R^{sd} is independently —CH₂—O—(CH₂)ₛₙ—X^{sc}, wherein X^{sc} is optionally substituted heterocyclyl. In certain embodiments, each instance of R^{sc} and R^{sd} is independently —CH₂—O—(CH₂)ₛₙ—X^{sc}, wherein X^{sc} is optionally substituted six-membered heterocyclyl (e.g. tetrahydropyran). In certain embodiments, X^{sc} is

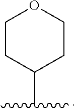 [SMILES: *C1CCOCC1]

In certain embodiments, X^{sc} is

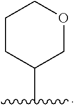 [SMILES: *C1CCCOC1]

In certain embodiments, X^{sc} is

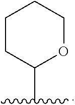 [SMILES: *C1CCCCO1]

In certain embodiments, each instance of R^{sc} and R^{sd} is independently —CH₂—O—(CH₂)ₛₘ—O—X^{sd}, wherein X^{sd} is optionally substituted C₁₋₆ alkyl. In certain embodiments, each instance of R^{sc} and R^{sd} is independently —CH₂—O—(CH₂)ₛₘ—O—X^{sd}, wherein X^{sd} is unsubstituted C₁₋₆ alkyl (e.g. methyl, ethyl, or n-propyl).

In certain embodiments of Formulae (S-i) and (S-i-a)-(S-i-c), each instance of R^{sc} and R^{sd} is independently unsubstituted C₁₋₆ alkyl; and R^{sb} is optionally substituted alkyl, —C(═O)N(R^{SN1})₂, or —OR^{SO}, wherein R^{SN1} and R^{SO} are as defined herein. In certain embodiments, each instance of R^{sc} and R^{sd} is independently unsubstituted C₁₋₆ alkyl; and R^{sb} is optionally substituted alkyl. In certain embodiments, each instance of R^{sc} and R^{sd} is independently unsubstituted C₁₋₆ alkyl; and R^{sb} is unsubstituted alkyl (e.g. methyl, ethyl, or n-propyl). In certain embodiments, each instance of R^{sc} and R^{sd} is independently unsubstituted C₁₋₆ alkyl; and R^{sb} is substituted alkyl. In certain embodiments, each instance of R^{sc} and R^{sd} is independently unsubstituted C₁₋₆ alkyl; and R^{sb} is —CH₂—O—(CH₂)ₛₙ—X^{sc} or —CH₂—O—(CH₂)ₛₘ—O—X^{sd}, wherein sn, sm, X^{sc}, and X^{sd} are as defined herein. In certain embodiments, each instance of R^{sc} and R^{sd} is independently unsubstituted C₁₋₆ alkyl; and R^{sb} is —OR^{SO}, wherein R^{SO} is as defined herein. In certain embodiments, each instance of R^{sc} and R^{sd} is independently unsubstituted C₁₋₆ alkyl; and R^{sb} is —O-methyl, —O-ethyl, —O-propyl, —O-isopropyl, —O— isobutyl, or —O-isoamyl.

In certain embodiments, R^{sc} and R^{sd} are taken together with the intervening atom to form an optionally substituted heterocyclyl. In certain embodiments, R^{sc} and R^{sd} are taken together with the intervening atom to form an optionally substituted heterocyclic ring of Formula (S-i-d):

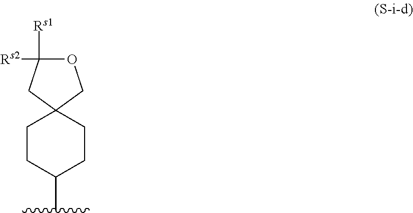

wherein each instance of R^{s1} and R^{s2} is independently optionally substituted C₁₋₆ alkyl.

In certain embodiments, R^{sc} and R^{sd} are taken together with the intervening atom to form an optionally substituted heterocyclic ring of one of the following formulae:

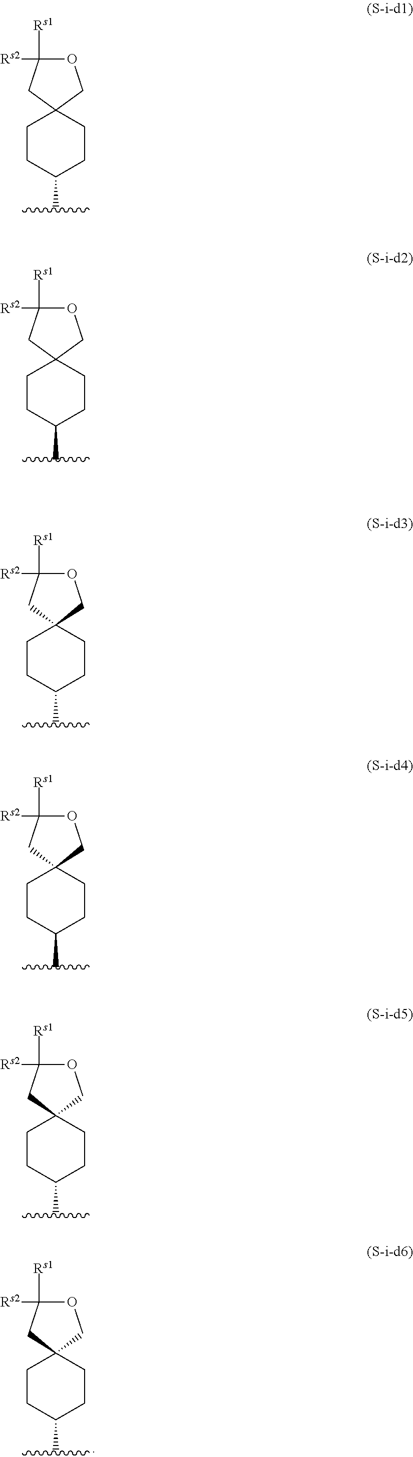

In some embodiments, each instance of R^{s1} and R^{s2} is independently unsubstituted C₁₋₆ alkyl. In some embodiments, both R^{s1} and R^{s2} are methyl.

In some embodiments, R^{sc} and R^{sd} are taken together with the intervening atom to form a substituted heterocyclic ring of Formula (S-i-e):

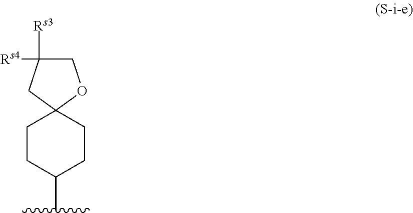

wherein each instance of R^{s3} and R^{s4} is independently optionally substituted C₁₋₆ alkyl.

In some embodiments, R^{sc} and R^{sd} are taken together with the intervening atom to form a substituted heterocyclic ring of one of the following formulae:

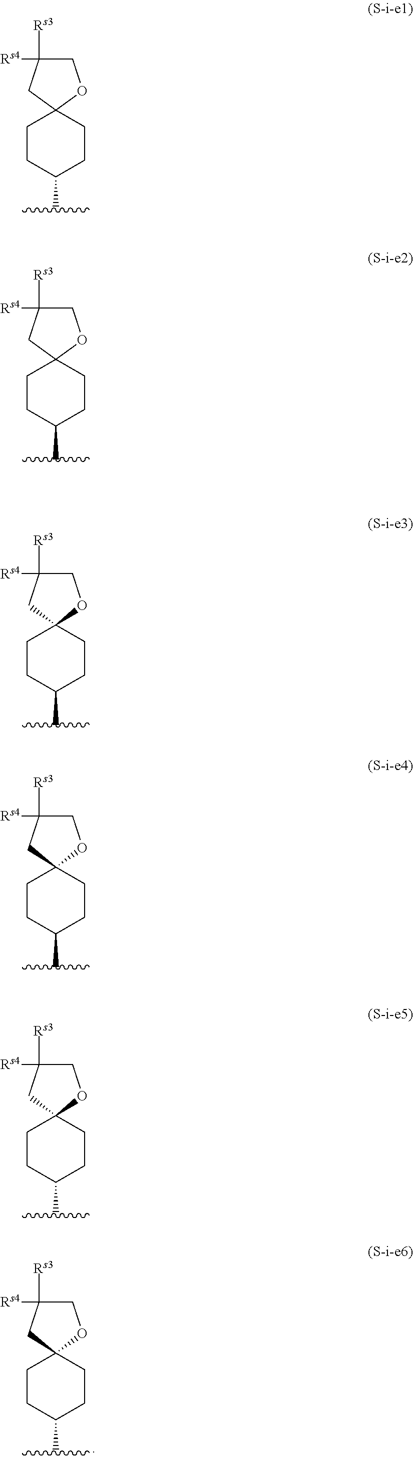

In some embodiments, each instance of R^{s3} and R^{s4} is independently unsubstituted C₁₋₆ alkyl. In some embodiments, both R^{s3} and R^{s4} are methyl.

In some embodiments, R^{sc} and R^{sd} are taken together with the intervening atom to form a substituted heterocyclic ring of Formula (S-i-f1):

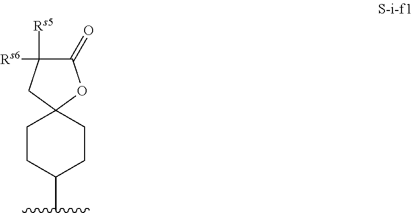

wherein each of R^{s5} and R^{s6} is independently C₁₋₆ alkyl.

In some embodiments, R^{sc} and R^{sd} are taken together with the intervening atom to form a substituted heterocyclic ring of one of the following formulae:

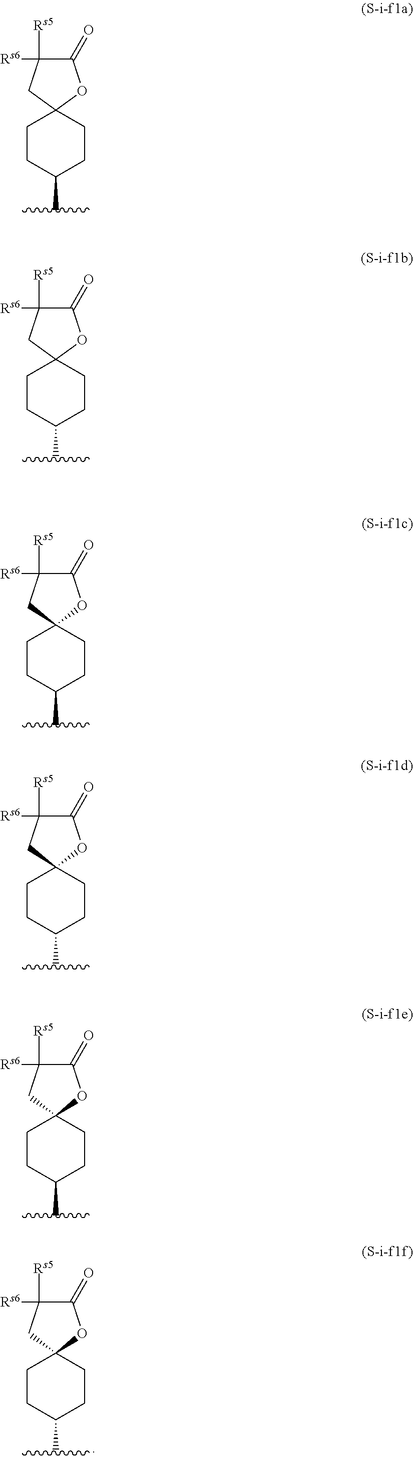

In some embodiments, R^{sc} and R^{sd} are taken together with the intervening atom to form a substituted heterocyclic ring of Formula (S-i-f2):

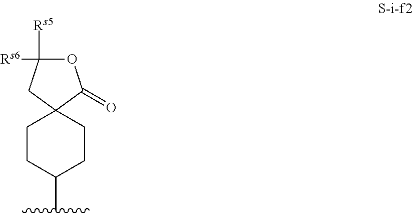

wherein each of R^{s5} and R^{s6} is independently C₁₋₆ alkyl.

In some embodiments, each of R^{s5} and R^{s6} is independently unsubstituted C₁₋₆ alkyl. In some embodiments, R^{s5} and R^{s6} are both methyl.

In some embodiments, R^{sc} and R^{sd} are taken together with the intervening atom to form a heterocyclic ring of Formula (S-i-g):

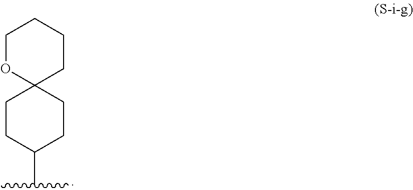 [SMILES: *C1CCC2(CCCCO2)CC1]

In some embodiments, R^{sc} and R^{sd} are taken together with the intervening atom to form a heterocyclic ring of one of the following formulae:

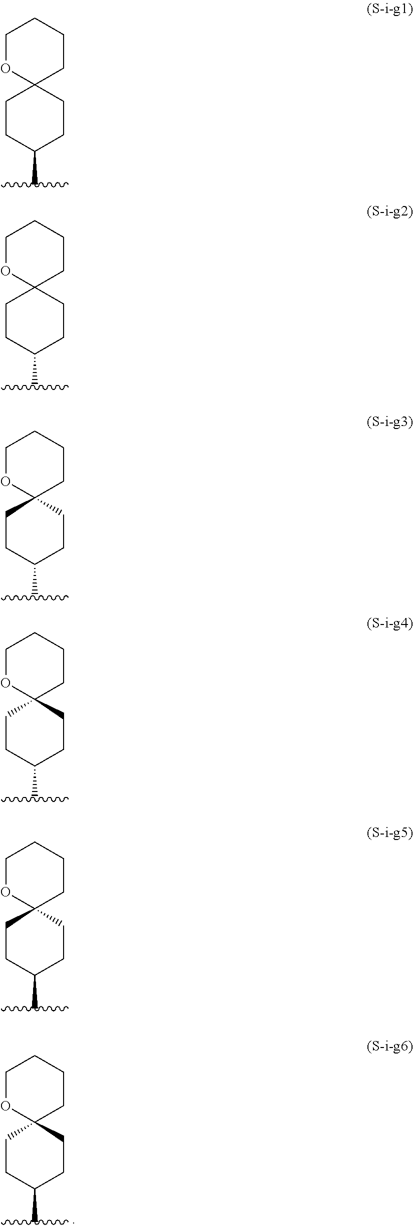

In some embodiments, R^{sc} and R^{sd} are taken together with the intervening atom to form a heterocyclic ring of Formula (S-i-h):

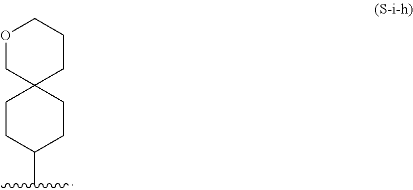 [SMILES: *C1CCC2(CCCOC2)CC1]

In some embodiments, R^{sc} and R^{sd} are taken together with the intervening atom to form a heterocyclic ring of one of the following formulae:

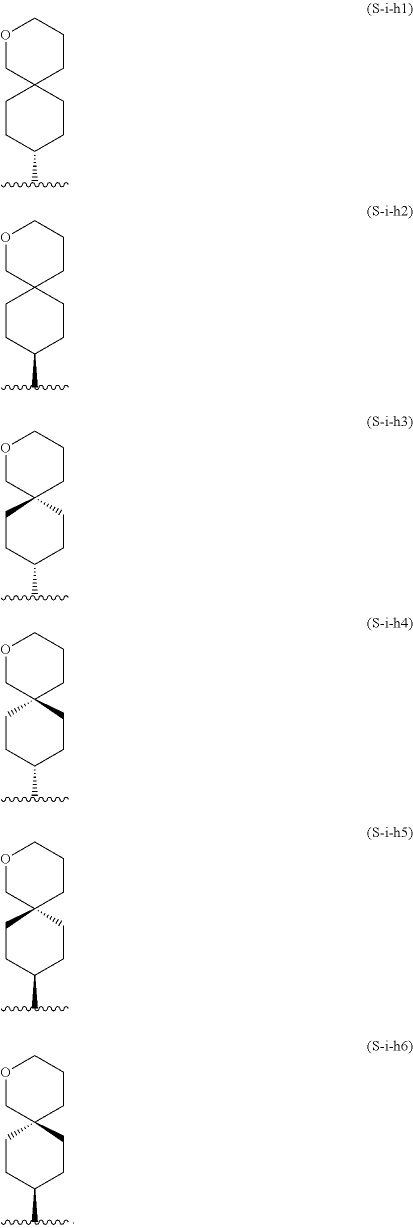

In some embodiments, R^{sc} and R^{sd} are taken together with the intervening atom to form a substituted heterocyclic ring of Formula (S-i-i):

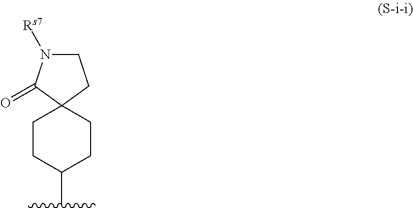

wherein each instance of R^{s7} is optionally substituted alkyl.

In some embodiments, R^{sc} and R^{sd} are taken together with the intervening atom to form a substituted heterocyclic ring of one of the following formulae:

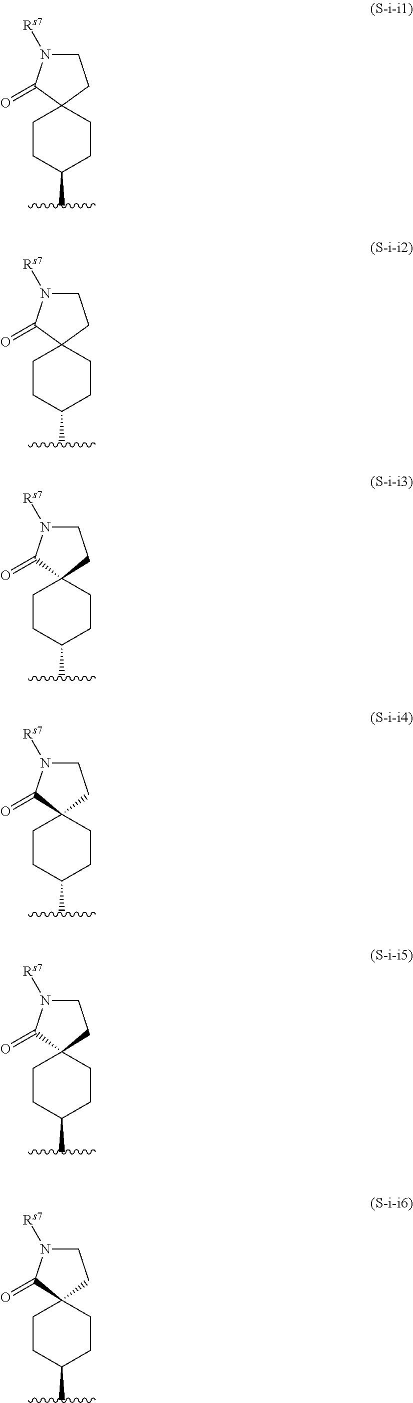

wherein each instance of R^{s7} is optionally substituted alkyl.

As generally defined herein, R^{s7} is optionally substituted alkyl. In certain embodiments, R^{s7} is optionally substituted C₄₋₈ alkyl. In certain embodiments, R^{s7} is unsubstituted C₄₋₈ alkyl. In certain embodiments, R^{s7} is n-butyl, s-butyl, t-butyl, n-pentyl, t-pentyl, neo-pentyl, i-pentyl, s-pentyl, or 3-pentyl.

In certain embodiments, R^{sc} and R^{sd} are taken together with the intervening atom to form a substituted heterocyclic ring of Formula (S-i-j):

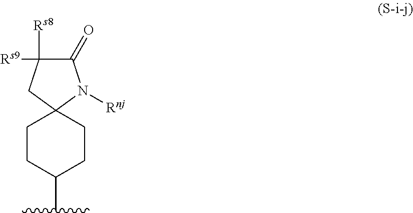

wherein each instance of R^{s8} and R^{s9} is independently hydrogen or optionally substituted C₁₋₆ alkyl; and R^{nj} is independently hydrogen, optionally substituted C₁₋₆ alkyl, or a nitrogen protecting group.

In certain embodiments, R^{sc} and R^{sd} are taken together with the intervening atom to form a substituted heterocyclic ring of one of the following formulae:

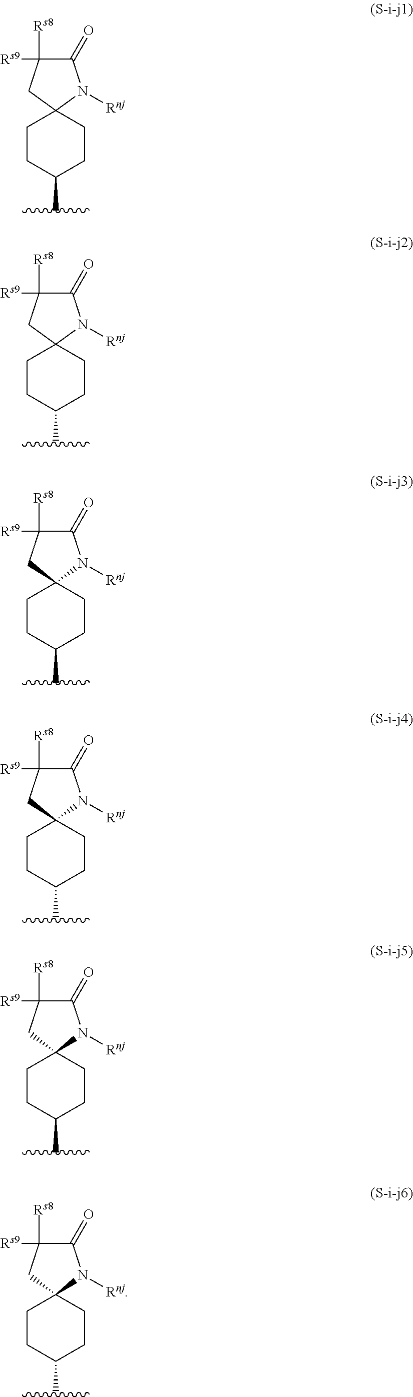

In certain embodiments, R^{s8} and R^{s9} are the same. In certain embodiments, R^{s8} and R^{s9} are different. In certain embodiments, each of R^{s8} and R^{s9} is independently unsubstituted C₁₋₆ alkyl. In certain embodiments, R^{s8} and R^{s9} are both methyl or ethyl.

As generally defined herein, R^{nj} is hydrogen, optionally substituted C₁₋₆ alkyl, or a nitrogen protecting group. In certain embodiments, R^{nj} is hydrogen. In certain embodiments, R^{nj} is unsubstituted C₁₋₆ alkyl (e.g. methyl or ethyl).

In certain embodiments, R^{s8} and R^{s9} is independently unsubstituted C₁₋₆ alkyl (e.g. methyl or ethyl); and R^{nj} is hydrogen. In certain embodiments, R^{s8} and R^{s9} is independently unsubstituted C₁₋₆ alkyl (e.g. methyl or ethyl); and R^{nj} is unsubstituted C₁₋₆ alkyl (e.g. methyl or ethyl).

In certain embodiments, R^{sc} and R^{sd} are taken together with the intervening atom to form a carbocyclic ring of Formula (S-i-l):

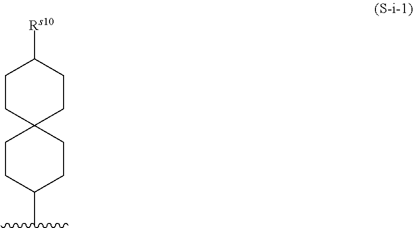

wherein R^{s10} is —OR^{sl}; and R^{sl} is optionally substituted alkyl.

In certain embodiments, R^{sc} and R^{sd} are taken together with the intervening atom to form a carbocyclic ring of one of the following formulae:

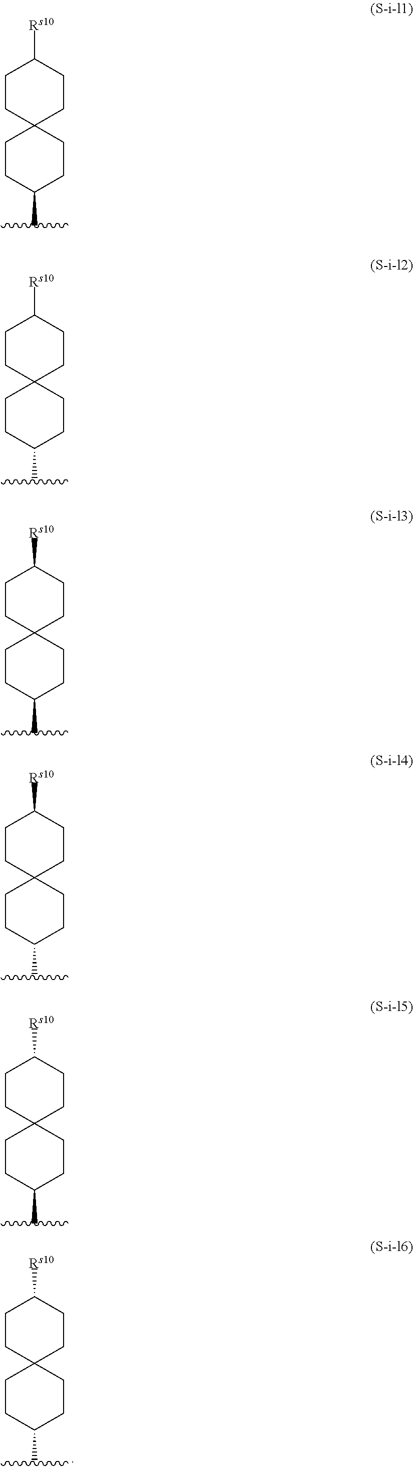

In certain embodiments, R^{s10} is —OR^{sl}; and R^{sl} is unsubstituted alkyl. In certain embodiments, R^{s10} is —OCH₃.

In certain embodiments, R^{sa} and R^{sb} are taken together with the intervening atom to form a carbocyclic ring of one of Formula (S-i-m):

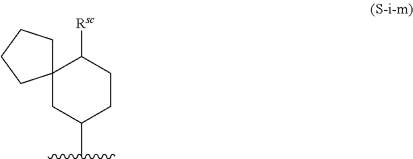

wherein R^{sc} is as defined herein.

In certain embodiments, R^{sa} and R^{sb} are taken together with the intervening atom to form a carbocyclic ring of one of the following formulae:

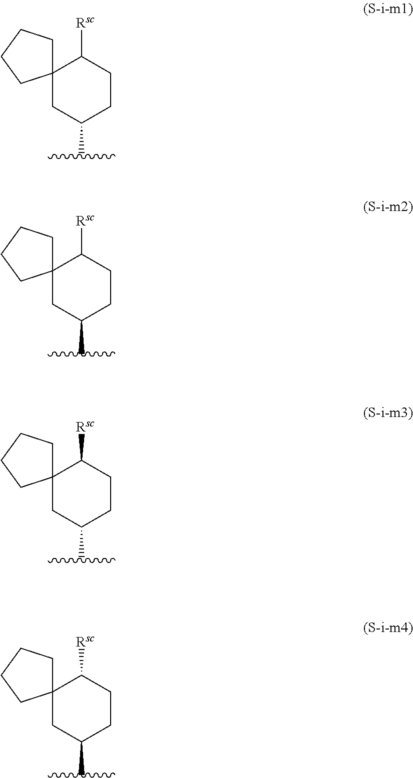

In certain embodiments of Formulae (S-i-m) and (S-i-m1)-(S-i-m4), R^{sc} is optionally substituted C₁₋₆ alkyl. In certain embodiments, R^{sc} is substituted C₁₋₆ alkyl. In certain embodiments, R^{sc} is independently —CH₂—O—(CH₂)ₛₙ—X^{sc}, wherein sn and X^{sc} are as defined herein. In certain embodiments, R^{sc} is independently —CH₂—O—(CH₂)ₛₙ—X^{sc}, wherein X^{sc} is unsubstituted C₁₋₆ alkyl. In certain embodiments, R^{sc} is independently —CH₂—O—(CH₂)ₛₙ—CH₃.

In certain embodiments, R^{W2} is of Formula (S-i-n):

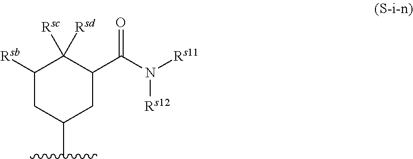

wherein R^{sb}, R^{sc}, and R^{sd} are as defined herein; each of R^{s11} and R^{s12} is independently hydrogen or optionally substituted C₁₋₆ alkyl.

In certain embodiments, R^{W2} is of one of the following formulae:

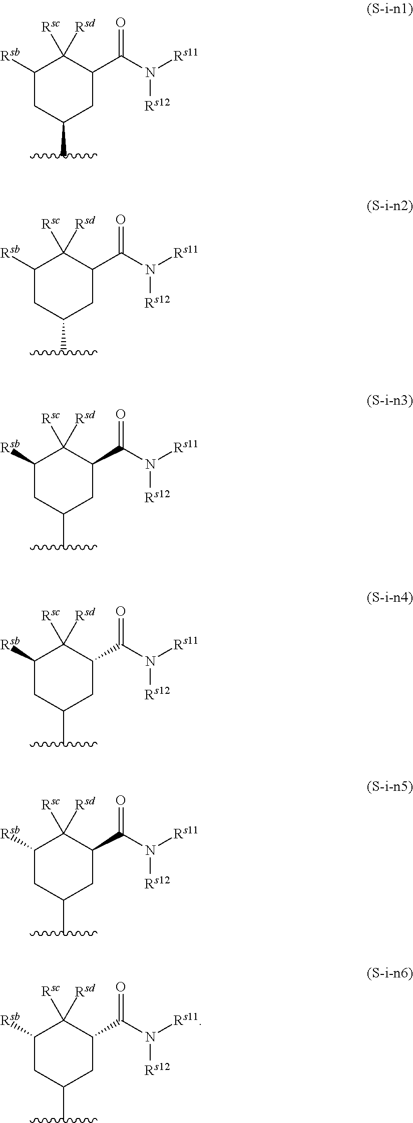

In certain embodiments, R^{s11} and R^{s12} are the same. In certain embodiments, R^{s11} and R^{s12} are different. In certain embodiments, R^{s11} is hydrogen and R^{s12} is independently substituted C₁₋₆ alkyl. In certain embodiments, R^{s11} is hydrogen and R^{s12} is independently unsubstituted C₁₋₆ alkyl. In certain embodiments, R^{s11} is hydrogen and R^{s12} is methyl, ethyl, n-propyl, i-propyl, n-butyl, s-butyl, t-butyl, n-pentyl, t-pentyl, neo-pentyl, i-pentyl, s-pentyl, or 3-pentyl).

In certain embodiments of Formulae (S-i-n) and (S-i-n1)-(S-i-n6), R^{sb} is optionally substituted C₁₋₆ alkyl. In certain embodiments of Formulae (S-i-n) and (S-i-n1)-(S-i-n6), R^{sb} is unsubstituted C₁₋₆ alkyl. In certain embodiments, R^{sb} is ethyl.

In certain embodiments of Formulae (S-i-n) and (S-i-n1)-(S-i-n6), R^{sc} and R^{sd} are the same. In certain embodiments of Formulae (S-i-n) and (S-i-n1)-(S-i-n6), R^{sc} and R^{sd} are different. In certain embodiments of Formulae (S-i-n) and (S-i-n1)-(S-i-n6), each of R^{sc} and R^{sd} is independently optionally substituted C₁₋₆ alkyl. In certain embodiments of Formulae (S-i-n) and (S-i-n1)-(S-i-n6), R^{sc} and R^{sd} are both methyl.

In certain embodiments, R^{W2} is of Formula (S-i-o):

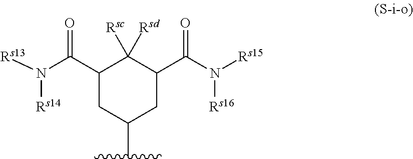

wherein each of R^{s13}, R^{s14}, R^{s15}, and R^{s16} is independently hydrogen or optionally substituted C₁₋₆ alkyl.

In certain embodiments, R^{W2} is of one of the following formulae:

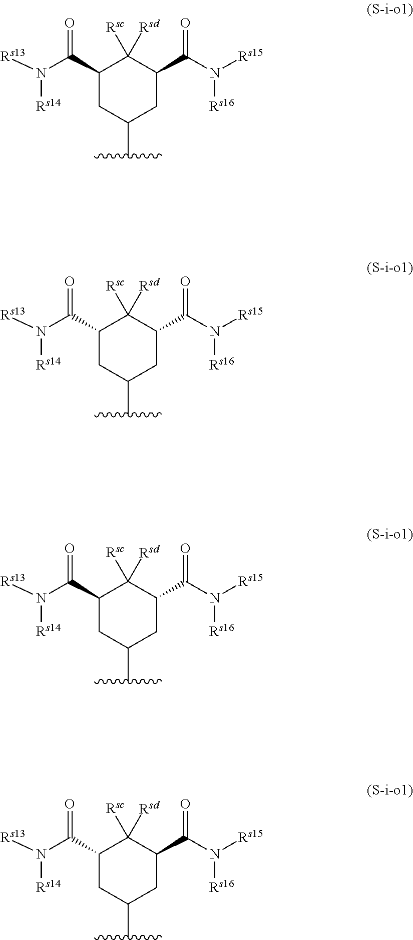

In certain embodiments, R^{s14} and R^{s16} are the same. In certain embodiments, R^{s14} and R^{s16} are different. In certain embodiments, each of R^{s14} and R^{s16} is independently optionally substituted C₁₋₆ alkyl. In certain embodiments, R^{s14} and R^{s16} are both unsubstituted C₁₋₆ alkyl. In certain embodiments, R^{s14} and R^{s16} are both methyl, isobutyl, or isopentyl. In certain embodiments, R^{s14} is hydrogen and R^{s16} is optionally substituted C₁₋₆ alkyl. In certain embodiments, R^{s14} is hydrogen and R^{s16} is substituted C₁₋₆ alkyl (e.g. methyl or ethyl). In certain embodiments, R^{s13} and R^{s15} are the same. In certain embodiments, R^{s13} and R^{s15} are different. In certain embodiments, each of R^{s13} and R^{s15} is independently optionally substituted C₁₋₆ alkyl. In certain embodiments, R^{s13} and R^{s15} are both unsubstituted C₁₋₆ alkyl. In certain embodiments, R^{s13} and R^{s15} are both isobutyl. In certain embodiments, each of R^{s13} and R^{s15} is independently unsubstituted C₁₋₆ alkyl. In certain embodiments, R^{s13} and R^{s15} are both isobutyl. In certain embodiments, R^{s13} is methyl or ethyl and R^{s15} is isopentyl. In certain embodiments, each of R^{s13} and R^{s15} is independently substituted C₁₋₆ alkyl. In certain embodiments, each of R^{s13} and R^{s15} is independently —(CH₂)_{sd}—OX^{sd}, wherein sd is 1, 2, 3, 4, or 5; and X^{sd} is optionally substituted C₁₋₆ alkyl. In certain embodiments, sd is 1. In certain embodiments, sd is 2. In certain embodiments, sd is 3. In certain embodiments, sd is 4. In certain embodiments, sd is 5. In certain embodiments, X^{sd} is substituted C₁₋₆ alkyl. In certain embodiments, X^{sd} is unsubstituted C₁₋₆ alkyl (e.g. methyl). In certain embodiments, R^{s13} and R^{s15} are both —(CH₂)₂—OCH₃.

In certain embodiments, R^{W2} is optionally substituted cyclohexenyl. In certain embodiments, R^{W2} is of Formula (S-ii):

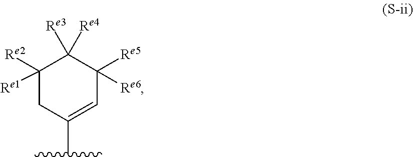

wherein

each of R^{e1}, R^{e2}, R^{e3}, R^{e4}, R^{e5}, and R^{e6} is independently hydrogen, optionally substituted C₁₋₆ alkyl, or —OR^{eo};

R^{eo} is hydrogen, optionally substituted alkyl, optionally substituted carbocyclyl, optionally substituted heterocyclyl, optionally substituted aryl, optionally substituted heteroaryl, or an oxygen protecting group; and

R^{e1} and R^{e2} are optionally taken with the intervening atom to form an optionally substituted carbocyclic ring; and

R^{e3} and R^{e4} are optionally taken with the intervening atom to form an optionally substituted carbocyclic ring.

In certain embodiments, R^{W2} is of one of the following formulae:

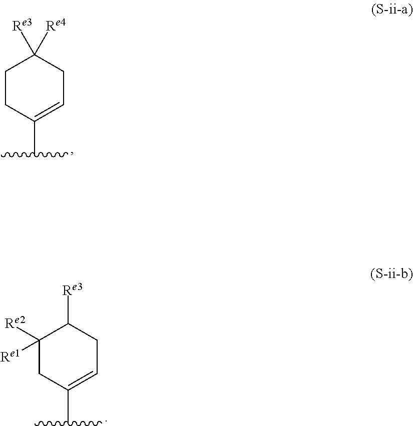

As generally defined herein, R^{e1} is independently hydrogen, optionally substituted C₁₋₆ alkyl, or —OR^{eo}. In certain embodiments, R^{e1} is hydrogen. In certain embodiments, R^{e1} is optionally substituted C₁₋₆ alkyl. In certain embodiments, R^{e1} is unsubstituted C₁₋₆ alkyl (e.g. methyl or ethyl). In certain embodiments, R^{e1} is substituted C₁₋₆ alkyl. In certain embodiments, R^{e1} is substituted C₁₋₆ haloalkyl. In certain embodiments, R^{e1} is —CH₂—CF₃. In certain embodiments, R^{e1} is —CH₂—O—X^{e1}, wherein X^{e1} is optionally substituted C₁₋₆ alkyl. In certain embodiments, R^{e1} is —CH₂—O—X^{e1}, wherein X^{e1} is unsubstituted C₁₋₆ alkyl (e.g. methyl or ethyl). In certain embodiments, R^{e1} is —CH₂—O—X^{e1}, wherein X^{e1} is substituted C₁₋₆ alkyl. In certain embodiments, R^{e1} is —CH₂—O—X^{e1}, wherein X^{e1} is substituted C₁₋₆ haloalkyl. In certain embodiments, R^{e1} is —CH₂—O—X^{e1}, wherein X^{e1} is —CH₂—CF₃. In certain embodiments, R^{e1} is —O—R^{eo}, wherein R^{eo} is optionally substituted C₁₋₆ alkyl. In certain embodiments, R^{e1} is —O—R^{eo}, wherein R^{eo} is unsubstituted C₁₋₆ alkyl (e.g. methyl or ethyl). In certain embodiments, R^{e1} is —O—R^{eo}, wherein R^{eo} is substituted C₁₋₆ alkyl (e.g. haloalkyl). In certain embodiments, R^{e1} is —O—(CH₂)_{eb}—O—X^{e2}; X^{e2} is optionally substituted C₁₋₆ alkyl; and eb is 1, 2, 3, 4, 5, or 6. In certain embodiments, R^{e1} is —O—(CH₂)_{eb}—O—X^{e2}; and X^{e2} is unsubstituted C₁₋₆ alkyl (e.g. methyl or ethyl). In certain embodiments, eb is 1. In certain embodiments, eb is 2. In certain embodiments, eb is 3. In certain embodiments, eb is 4. In certain embodiments, eb is 5. In certain embodiments, eb is 6. In certain embodiments, R^{e1} is —O—(CH₂)₃—O—CH₃.

As generally defined herein, R^{e2} is independently hydrogen, optionally substituted C₁₋₆ alkyl, or —OR^{eo}. In certain embodiments, R^{e2} is hydrogen. In certain embodiments, R^{e2} is optionally substituted C₁₋₆ alkyl. In certain embodiments, R^{e2} is unsubstituted C₁₋₆ alkyl (e.g. methyl or ethyl). In certain embodiments, R^{e2} is substituted C₁₋₆ alkyl. In certain embodiments, R^{e2} is substituted C₁₋₆ haloalkyl. In certain embodiments, R^{e2} is —CH₂—CF₃. In certain embodiments, R^{e2} is —CH₂—O—X^{e2}, wherein X^{e2} is optionally substituted C₁₋₆ alkyl. In certain embodiments, R^{e2} is —CH₂—O—X^{e2}, wherein X^{e2} is unsubstituted C₁₋₆ alkyl (e.g. methyl or ethyl). In certain embodiments, R^{e2} is —CH₂—O—X^{e2}, wherein X^{e2} is substituted C₁₋₆ alkyl. In certain embodiments, R^{e2} is —CH₂—O—X^{e2}, wherein X^{e2} is substituted C₁₋₆ haloalkyl. In certain embodiments, R^{e2} is —CH₂—O—X^{e2}, wherein X^{e2} is —CH₂—CF₃. In certain embodiments, R^{e2} is —O—R^{eo}, wherein R^{eo} is optionally substituted C₁₋₆ alkyl. In certain embodiments, R^{e2} is —O—R^{eo}, wherein R^{eo} is unsubstituted C₁₋₆ alkyl (e.g. methyl or ethyl). In certain embodiments, R^{e2} is —O—R^{eo}, wherein R^{eo} is substituted C₁₋₆ alkyl (e.g. haloalkyl). In certain embodiments, R^{e2} is —O—(CH₂)_{eb}—O—X^{e2}; X^{e2} is optionally substituted C₁₋₆ alkyl; and eb is 1, 2, 3, 4, 5, or 6. In certain embodiments, R^{e2} is —O—(CH₂)_{eb}—O—X^{e2}; and X^{e2} is unsubstituted C₁₋₆ alkyl (e.g. methyl or ethyl). In certain embodiments, eb is 1. In certain embodiments, eb is 2. In certain embodiments, eb is 3. In certain embodiments, eb is 4. In certain embodiments, eb is 5. In certain embodiments, eb is 6. In certain embodiments, R^{e2} is —O—(CH₂)₃—O—CH₃.

As generally defined herein, R^{e3} is independently hydrogen, optionally substituted C₁₋₆ alkyl, or —OR^{eo}. In certain embodiments, R^{e3} is hydrogen. In certain embodiments, R^{e3} is optionally substituted C₁₋₆ alkyl. In certain embodiments, R^{e3} is unsubstituted C₁₋₆ alkyl (e.g. methyl or ethyl). In certain embodiments, R^{e3} is substituted C₁₋₆ alkyl. In certain embodiments, R^{e3} is substituted C₁₋₆ haloalkyl. In certain embodiments, R^{e3} is —CH₂—CF₃. In certain embodiments, R^{e3} is —CH₂—O—X^{e3}, wherein X^{e3} is optionally substituted C₁₋₆ alkyl. In certain embodiments, R^{e3} is —CH₂—O—X^{e3}, wherein X^{e3} is unsubstituted C₁₋₆ alkyl (e.g. methyl or ethyl). In certain embodiments, R^{e3} is —CH₂—O—X^{e3}, wherein X^{e3} is substituted C₁₋₆ alkyl. In certain embodiments, R^{e3} is —CH₂—O—X^{e3}, wherein X^{e3} is substituted C₁₋₆ haloalkyl. In certain embodiments, R^{e3} is —CH₂—O—X^{e3}, wherein X^{e3} is —CH₂—CF₃. In certain embodiments, R^{e3} is —O—R^{eo}, wherein R^{eo} is optionally substituted C₁₋₆ alkyl. In certain embodiments, R^{e3} is —O—R^{eo}, wherein R^{eo} is unsubstituted C₁₋₆ alkyl (e.g. methyl or ethyl). In certain embodiments, R^{e3} is —O—R^{eo}, wherein R^{eo} is substituted C₁₋₆ alkyl (e.g. haloalkyl). In certain embodiments, R^{e3} is —O—(CH₂)_{eb}—O—X^{e2}; X^{e2} is optionally substituted C₁₋₆ alkyl; and eb is 1, 2, 3, 4, 5, or 6. In certain embodiments, R^{e3} is —O—(CH₂)_{eb}—O—X^{e2}; and X^{e2} is unsubstituted C₁₋₆ alkyl (e.g. methyl or ethyl). In certain embodiments, eb is 1. In certain embodiments, eb is 2. In certain embodiments, eb is 3. In certain embodiments, eb is 4. In certain embodiments, eb is 5. In certain embodiments, eb is 6. In certain embodiments, R^{e3} is —O—(CH₂)₃—O—CH₃.

As generally defined herein, R^{e4} is independently hydrogen, optionally substituted C₁₋₆ alkyl, or —OR^{eo}. In certain embodiments, R^{e4} is hydrogen. In certain embodiments, R^{e4} is optionally substituted C₁₋₆ alkyl. In certain embodiments, R^{e4} is unsubstituted C₁₋₆ alkyl (e.g. methyl or ethyl). In certain embodiments, R^{e4} is substituted C₁₋₆ alkyl. In certain embodiments, R^{e4} is substituted C₁₋₆ haloalkyl. In certain embodiments, R^{e4} is —CH₂—CF₃. In certain embodiments, R^{e4} is —CH₂—O—X^{e4}, wherein X^{e4} is optionally substituted C₁₋₆ alkyl. In certain embodiments, R^{e4} is —CH₂—O—X^{e4}, wherein X^{e4} is unsubstituted C₁₋₆ alkyl (e.g. methyl or ethyl). In certain embodiments, R^{e4} is —CH₂—O—X^{e4}, wherein X^{e4} is substituted C₁₋₆ alkyl. In certain embodiments, R^{e4} is —CH₂—O—X^{e4}, wherein X^{e4} is substituted C₁₋₆ haloalkyl. In certain embodiments, R^{e4} is —CH₂—O—X^{e4}, wherein X^{e4} is —CH₂—CF₃. In certain embodiments, R^{e4} is —O—R^{eo}, wherein R^{eo} is optionally substituted C₁₋₆ alkyl. In certain embodiments, R^{e4} is —O—R^{eo}, wherein R^{eo} is unsubstituted C₁₋₆ alkyl (e.g. methyl or ethyl). In certain embodiments, R^{e4} is —O—R^{eo}, wherein R^{eo} is substituted C₁₋₆ alkyl (e.g. haloalkyl). In certain embodiments, R^{e4} is —O—(CH₂)_{eb}—O—X^{e2}; X^{e2} is optionally substituted C₁₋₆ alkyl; and eb is 1, 2, 3, 4, 5, or 6. In certain embodiments, R^{e4} is —O—(CH₂)_{eb}—O—X^{e2}; and X^{e2} is unsubstituted C₁₋₆ alkyl (e.g. methyl or ethyl). In certain embodiments, eb is 1. In certain embodiments, eb is 2. In certain embodiments, eb is 3. In certain embodiments, eb is 4. In certain embodiments, eb is 5. In certain embodiments, eb is 6. In certain embodiments, R^{e4} is —O—(CH₂)₃—O—CH₃.

As generally defined herein, R^{e5} is independently hydrogen, optionally substituted C₁₋₆ alkyl, or —OR^{eo}. In certain embodiments, R^{e5} is hydrogen. In certain embodiments, R^{e5} is optionally substituted C₁₋₆ alkyl. In certain embodiments, R^{e5} is unsubstituted C₁₋₆ alkyl (e.g. methyl or ethyl). In certain embodiments, R^{e5} is substituted C₁₋₆ alkyl. In certain embodiments, R^{e5} is substituted C₁₋₆ haloalkyl. In certain embodiments, R^{e5} is —CH₂—CF₃. In certain embodiments, R^{e5} is —CH₂—O—X^{e5}, wherein X^{e5} is optionally substituted C₁₋₆ alkyl. In certain embodiments, R^{e5} is —CH₂—O—X^{e5}, wherein X^{e5} is unsubstituted C₁₋₆ alkyl (e.g. methyl or ethyl). In certain embodiments, R^{e5} is —CH₂—O—X^{e5}, wherein X^{e5} is substituted C₁₋₆ alkyl. In certain embodiments, R^{e5} is —CH₂—O—X^{e5}, wherein X^{e5} is substituted C₁₋₆ haloalkyl. In certain embodiments, R^{e5} is —CH₂—O—X^{e5}, wherein X^{e5} is —CH₂—CF₃. In certain embodiments, R^{e5} is —O—R^{eo}, wherein R^{eo} is optionally substituted C₁₋₆ alkyl. In certain embodiments, R^{e5} is —O—R^{eo}, wherein R^{eo} is unsubstituted C₁₋₆ alkyl (e.g. methyl or ethyl). In certain embodiments, R^{e5} is —O—R^{eo}, wherein R^{eo} is substituted C₁₋₆ alkyl (e.g. haloalkyl). In certain embodiments, R^{e5} is —O—(CH₂)_{eb}—O—X^{e2}; X^{e2} is optionally substituted C₁₋₆ alkyl; and eb is 1, 2, 3, 4, 5, or 6. In certain embodiments, R^{e5} is —O—(CH₂)_{eb}—O—X^{e2}; and X^{e2} is unsubstituted C₁₋₆ alkyl (e.g. methyl or ethyl). In certain embodiments, eb is 1. In certain embodiments, eb is 2. In certain embodiments, eb is 3. In certain embodiments, eb is 4. In certain embodiments, eb is 5. In certain embodiments, eb is 6. In certain embodiments, R^{e5} is —O—(CH₂)₃—O—CH₃.

As generally defined herein, R^{e6} is independently hydrogen, optionally substituted C₁₋₆ alkyl, or —OR^{eo}. In certain embodiments, R^{e6} is hydrogen. In certain embodiments, R^{e6} is optionally substituted C₁₋₆ alkyl. In certain embodiments, R^{e6} is unsubstituted C₁₋₆ alkyl (e.g. methyl or ethyl). In certain embodiments, R^{e6} is substituted C₁₋₆ alkyl. In certain embodiments, R^{e6} is substituted C₁₋₆ haloalkyl. In certain embodiments, R^{e6} is —CH₂—CF₃. In certain embodiments, R^{e6} is —CH₂—O—X^{e6}, wherein X^{e6} is optionally substituted C₁₋₆ alkyl. In certain embodiments, R^{e6} is —CH₂—O—X^{e6}, wherein X^{e6} is unsubstituted C₁₋₆ alkyl (e.g. methyl or ethyl). In certain embodiments, R^{e6} is —CH₂—O—X^{e6}, wherein X^{e6} is substituted C₁₋₆ alkyl. In certain embodiments, R^{e6} is —CH₂—O—X^{e6}, wherein X^{e6} is substituted C₁₋₆ haloalkyl. In certain embodiments, R^{e6} is —CH₂—O—X^{e6}, wherein X^{e6} is —CH₂—CF₃. In certain embodiments, R^{e6} is —O—R^{eo}, wherein R^{eo} is optionally substituted C₁₋₆ alkyl. In certain embodiments, R^{e6} is —O—R^{eo}, wherein R^{eo} is unsubstituted C₁₋₆ alkyl (e.g. methyl or ethyl). In certain embodiments, R^{e6} is —O—R^{eo}, wherein R^{eo} is substituted C₁₋₆ alkyl (e.g. haloalkyl). In certain embodiments, R^{e6} is —O—(CH₂)_{eb}—O—X^{e2}; X^{e2} is optionally substituted C₁₋₆ alkyl; and eb is 1, 2, 3, 4, 5, or 6. In certain embodiments, R^{e6} is —O—(CH₂)_{eb}—O—X^{e2}; and X^{e2} is unsubstituted C₁₋₆ alkyl (e.g. methyl or ethyl). In certain embodiments, eb is 1. In certain embodiments, eb is 2. In certain embodiments, eb is 3. In certain embodiments, eb is 4. In certain embodiments, eb is 5. In certain embodiments, eb is 6. In certain embodiments, R^{e6} is —O—(CH₂)₃—O—CH₃.

In certain embodiments, R^{e3} and R^{e4} are taken with the intervening atom to form an optionally substituted heterocyclyl. In certain embodiments, R^{W2} is of Formula (S-ii-a1):

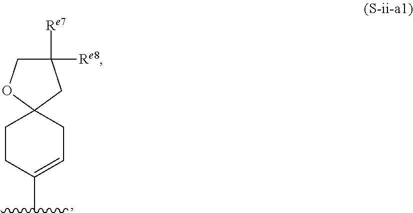

wherein each of R^{e7} and R^{e8} is independently optionally substituted C₁₋₆ alkyl.

In certain embodiments, R^{e7} and R^{e8} are the same. In certain embodiments, R^{e7} and R^{e8} are different. In certain embodiments, R^{e7} and R^{e8} are both unsubstituted C₁₋₆ alkyl. In certain embodiments, R^{e7} and R^{e8} are both methyl.

In certain embodiments, R^{W2} is optionally substituted tetrahydropyran. In certain embodiments, R^{W2} is of Formula (S-iii):

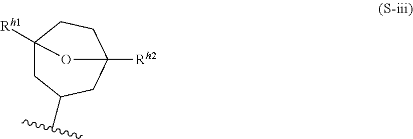

wherein each of R^{h1} and R^{h2} is independently hydrogen, halo, —CN, NO₂, or optionally substituted C₁₋₆ alkyl.

In certain embodiments, R^{h1} and R^{h2} are the same. In certain embodiments, R^{h1} and R^{h2} are different. In certain embodiments, R^{h1} and R^{h2} are both unsubstituted C₁₋₆ alkyl. In certain embodiments, R^{h1} and R^{h2} are both ethyl.

In certain embodiments, R^{W1} is substituted cyclohexyl of Formula (S-iii):

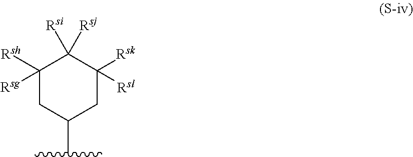

wherein

each of R^{sg}, R^{sh}, R^{sk}, and R^{sl} is independently hydrogen or optionally substituted C₁₋₆ alkyl,

each of R^{si} and R^{sj} is independently optionally substituted C₁₋₆ alkyl; and

R^{sg} and R^{sh} are optionally taken together with the intervening atom to form an optionally substituted carbocylic ring or an optionally substituted heterocyclic ring;

R^{si} and R^{sj} are optionally taken together with the intervening atom to form an optionally substituted carbocylic ring or an optionally substituted heterocyclic ring; and

R^{sk} and R^{sl} are optionally taken together with the intervening atom to form an optionally substituted carbocylic ring or an optionally substituted heterocyclic ring.

In certain embodiments, R^{si} and R^{sj} are the same. In certain embodiments, R^{si} and R^{sj} are different.

As generally defined herein, R^{sg} is independently hydrogen or optionally substituted C₁₋₆ alkyl. In certain embodiments, R^{sg} is hydrogen. In certain embodiments, R^{sg} is optionally substituted C₁₋₆ alkyl. In certain embodiments, R^{sg} is unsubstituted C₁₋₆ alkyl (e.g. methyl, ethyl, n-propyl, or iso-propyl). In certain embodiments, R^{sg} is substituted C₁₋₆ alkyl. In certain embodiments, R^{sg} is —CH₂—O—(CH₂)ₛₚ—X^{si} or —CH₂—O—(CH₂)_{sq}—O—X^{sj}; wherein sp is 0, 1, 2, 3, 4, 5, or 6; sq is 1, 2, 3, 4, 5, or 6; X^{si} is hydrogen, optionally substituted C₁₋₆ alkyl, optionally substituted aryl, optionally substituted carbocyclyl, or optionally substituted heterocyclyl; and X^{sj} is optionally substituted C₁₋₆ alkyl, optionally substituted aryl, optionally substituted carbocyclyl, or optionally substituted heterocyclyl. In certain embodiments, R^{sg} is —CH₂—O—(CH₂)ₛₚ—X^{si}, wherein X^{si} is hydrogen or optionally substituted C₁₋₆ alkyl. In certain embodiments, R^{sg} is —CH₂—O—X^{si}, wherein X^{si} is hydrogen or optionally substituted C₁₋₆ alkyl. In certain embodiments, X^{si} is unsubstituted C₁₋₆ alkyl (e.g. methyl or ethyl). In certain embodiments, X^{si} is substituted C₁₋₆ alkyl. In certain embodiments, X^{si} is C₁₋₆ haloalkyl (e.g. CF₃ or CH₂CF₃). In certain embodiments, R^{sg} is —CH₂—O—(CH₂)_{sq}—O—X^{sj}, wherein X^{sj} is hydrogen or optionally substituted C₁₋₆ alkyl. In certain embodiments, X^{si} is unsubstituted C₁₋₆ alkyl (e.g. methyl or ethyl). In certain embodiments, X^{sj} is substituted C₁₋₆ alkyl (e.g. CF₃ or CH₂CF₃).

As generally defined herein, R^{sh} is independently hydrogen or optionally substituted C₁₋₆ alkyl. In certain embodiments, R^{sh} is hydrogen. In certain embodiments, R^{sh} is optionally substituted C₁₋₆ alkyl. In certain embodiments, R^{sh} is unsubstituted C₁₋₆ alkyl (e.g. methyl, ethyl, n-propyl, or iso-propyl). In certain embodiments, R^{sh} is substituted C₁₋₆ alkyl. In certain embodiments, R^{sh} is —CH₂—O—(CH₂)ₛₚ—X^{si} or —CH₂—O—(CH₂)_{sq}—O—X^{sj}; wherein sp is 0, 1, 2, 3, 4, 5, or 6; sq is 1, 2, 3, 4, 5, or 6; X^{si} is hydrogen, optionally substituted C₁₋₆ alkyl, optionally substituted aryl, optionally substituted carbocyclyl, or optionally substituted heterocyclyl; and X^{sj} is optionally substituted C₁₋₆ alkyl, optionally substituted aryl, optionally substituted carbocyclyl, or optionally substituted heterocyclyl. In certain embodiments, R^{sh} is —CH₂—O—(CH₂)ₛₚ—X^{si}, wherein X^{si} is hydrogen or optionally substituted C₁₋₆ alkyl. In certain embodiments, R^{sh} is —CH₂—O—X^{si}, wherein X^{si} is hydrogen or optionally substituted C₁₋₆ alkyl. In certain embodiments, X^{si} is unsubstituted C₁₋₆ alkyl (e.g. methyl or ethyl). In certain embodiments, X^{si} is substituted C₁₋₆ alkyl. In certain embodiments, X^{si} is C₁₋₆ haloalkyl (e.g. CF₃ or CH₂CF₃). In certain embodiments, R^{sh} is —CH₂—O—(CH₂)_{sq}—O—X^{sj}, wherein X^{sj} is hydrogen or optionally substituted C₁₋₆ alkyl. In certain embodiments, X^{sj} is unsubstituted C₁₋₆ alkyl (e.g. methyl or ethyl). In certain embodiments, X^{sj} is substituted C₁₋₆ alkyl (e.g. CF₃ or CH₂CF₃).

As generally defined herein, R^{sk} is independently hydrogen or optionally substituted C₁₋₆ alkyl. In certain embodiments, R^{sk} is hydrogen. In certain embodiments, R^{sk} is optionally substituted C₁₋₆ alkyl. In certain embodiments, R^{sk} is unsubstituted C₁₋₆ alkyl (e.g. methyl, ethyl, n-propyl, or iso-propyl). In certain embodiments, R^{sk} is substituted C₁₋₆ alkyl. In certain embodiments, R^{sk} is —CH₂—O—(CH₂)ₛₚ—X^{si} or —CH₂—O—(CH₂)_{sq}—O—X^{sj}; wherein sp is 0, 1, 2, 3, 4, 5, or 6; sq is 1, 2, 3, 4, 5, or 6; X^{si} is hydrogen, optionally substituted C₁₋₆ alkyl, optionally substituted aryl, optionally substituted carbocyclyl, or optionally substituted heterocyclyl; and X^{sj} is optionally substituted C₁₋₆ alkyl, optionally substituted aryl, optionally substituted carbocyclyl, or optionally substituted heterocyclyl. In certain embodiments, R^{sk} is —CH₂—O—(CH₂)ₛₚ—X^{si}, wherein X^{si} is hydrogen or optionally substituted C₁₋₆ alkyl. In certain embodiments, R^{sk} is —CH₂—O—X^{si}, wherein X^{si} is hydrogen or optionally substituted C₁₋₆ alkyl. In certain embodiments, X^{si} is unsubstituted C₁₋₆ alkyl (e.g. methyl or ethyl). In certain embodiments, X^{si} is substituted C₁₋₆ alkyl. In certain embodiments, X^{si} is C₁₋₆ haloalkyl (e.g. CF₃ or CH₂CF₃). In certain embodiments, R^{sk} is —CH₂—O—(CH₂)_{sq}—O—X^{sj}, wherein X^{sj} is hydrogen or optionally substituted C₁₋₆ alkyl. In certain embodiments, X^{sj} is unsubstituted C₁₋₆ alkyl (e.g. methyl or ethyl). In certain embodiments, X^{sj} is substituted C₁₋₆ alkyl (e.g. CF₃ or CH₂CF₃).

As generally defined herein, R^{sl} is independently hydrogen or optionally substituted C₁₋₆ alkyl. In certain embodiments, R^{sl} is hydrogen. In certain embodiments, R^{sl} is optionally substituted C₁₋₆ alkyl. In certain embodiments, R^{sl} is unsubstituted C₁₋₆ alkyl (e.g. methyl, ethyl, n-propyl, or iso-propyl). In certain embodiments, R^{sl} is substituted C₁₋₆ alkyl. In certain embodiments, R^{sl} is —CH₂—O—(CH₂)ₛₚ—X^{si} or —CH₂—O—(CH₂)_{sq}—O—X^{sj}; wherein sp is 0, 1, 2, 3, 4, 5, or 6; sq is 1, 2, 3, 4, 5, or 6; X^{si} is hydrogen, optionally substituted C₁₋₆ alkyl, optionally substituted aryl, optionally substituted carbocyclyl, or optionally substituted heterocyclyl; and X^{sj} is optionally substituted C₁₋₆ alkyl, optionally substituted aryl, optionally substituted carbocyclyl, or optionally substituted heterocyclyl. In certain embodiments, R^{sl} is —CH₂—O—(CH₂)ₛₚ—X^{si}, wherein X^{si} is hydrogen or optionally substituted C₁₋₆ alkyl. In certain embodiments, R^{sl} is —CH₂—O—X^{si}, wherein X^{si} is hydrogen or optionally substituted C₁₋₆ alkyl. In certain embodiments, X^{si} is unsubstituted C₁₋₆ alkyl (e.g. methyl or ethyl). In certain embodiments, X^{si} is substituted C₁₋₆ alkyl. In certain embodiments, X^{si} is C₁₋₆ haloalkyl (e.g. CF₃ or CH₂CF₃). In certain embodiments, R^{sl} is —CH₂—O—(CH₂)_{sq}—O—X^{sj} wherein X^{sj} is hydrogen or optionally substituted C₁₋₆ alkyl. In certain embodiments, X^{sj} is unsubstituted C₁₋₆ alkyl (e.g. methyl or ethyl). In certain embodiments, X^{sj} is substituted C₁₋₆ alkyl (e.g. CF₃ or CH₂CF₃).

As generally defined herein, R^{si} is independently optionally substituted C₁₋₆ alkyl. In certain embodiments, R^{si} is optionally substituted C₁₋₆ alkyl. In certain embodiments, R^{si} is unsubstituted C₁₋₆ alkyl (e.g. methyl, ethyl, n-propyl, or iso-propyl). In certain embodiments, R^{si} is substituted C₁₋₆ alkyl. In certain embodiments, R^{si} is —CH₂—O—(CH₂)ₛₚ—X^{si} or —CH₂—O—(CH₂)_{sq}—O—X^{sj}; wherein sp is 0, 1, 2, 3, 4, 5, or 6; sq is 1, 2, 3, 4, 5, or 6; X^{si} is hydrogen, optionally substituted C₁₋₆ alkyl, optionally substituted aryl, optionally substituted carbocyclyl, or optionally substituted heterocyclyl; and X^{sj} is optionally substituted C₁₋₆ alkyl, optionally substituted aryl, optionally substituted carbocyclyl, or optionally substituted heterocyclyl. In certain embodiments, R^{si} is —CH₂—O—(CH₂)ₛₚ—X^{si}, wherein X^{si} is hydrogen or optionally substituted C₁₋₆ alkyl. In certain embodiments, R^{si} is —CH₂—O—X^{si}, wherein X^{si} is hydrogen or optionally substituted C₁₋₆ alkyl. In certain embodiments, X^{si} is unsubstituted C₁₋₆ alkyl (e.g. methyl or ethyl). In certain embodiments, X^{si} is substituted C₁₋₆ alkyl. In certain embodiments, X^{si} is C₁₋₆ haloalkyl (e.g. CF₃ or CH₂CF₃). In certain embodiments, R^{si} is —CH₂—O—(CH₂)_{sq}—O—X^{sj}, wherein X^{sj} is hydrogen or optionally substituted C₁₋₆ alkyl. In certain embodiments, X^{sj} is unsubstituted C₁₋₆ alkyl (e.g. methyl or ethyl). In certain embodiments, X^{sj} is substituted C₁₋₆ alkyl (e.g. CF₃ or CH₂CF₃).

As generally defined herein, R^{sj} is independently optionally substituted C₁₋₆ alkyl. In certain embodiments, R^{sj} is optionally substituted C₁₋₆ alkyl. In certain embodiments, R^{sj} is unsubstituted C₁₋₆ alkyl (e.g. methyl, ethyl, n-propyl, or iso-propyl). In certain embodiments, R^{sj} is substituted C₁₋₆ alkyl. In certain embodiments, R^{sj} is —CH₂—O—(CH₂)ₛₚ—X^{si} or —CH₂—O—(CH₂)_{sq}—O—X^{sj}; wherein sp is 0, 1, 2, 3, 4, 5, or 6; sq is 1, 2, 3, 4, 5, or 6; X^{si} is hydrogen, optionally substituted C₁₋₆ alkyl, optionally substituted aryl, optionally substituted carbocyclyl, or optionally substituted heterocyclyl; and X^{sj} is optionally substituted C₁₋₆ alkyl, optionally substituted aryl, optionally substituted carbocyclyl, or optionally substituted heterocyclyl. In certain embodiments, R^{sj} is —CH₂—O—(CH₂)ₛₚ—X^{si}, wherein X^{si} is hydrogen or optionally substituted C₁₋₆ alkyl. In certain embodiments, R^{sj} is —CH₂—O—X^{si}, wherein X^{si} is hydrogen or optionally substituted C₁₋₆ alkyl. In certain embodiments, X^{si} is unsubstituted C₁₋₆ alkyl (e.g. methyl or ethyl). In certain embodiments, X^{si} is substituted C₁₋₆ alkyl. In certain embodiments, X^{si} is C₁₋₆ haloalkyl (e.g. CF₃ or CH₂CF₃). In certain embodiments, R^{sj} is —CH₂—O—(CH₂)_{sq}—O—X^{sj}, wherein X^{sj} is hydrogen or optionally substituted C₁₋₆ alkyl. In certain embodiments, X^{sj} is unsubstituted C₁₋₆ alkyl (e.g. methyl or ethyl). In certain embodiments, X^{sj} is substituted C₁₋₆ alkyl (e.g. CF₃ or CH₂CF₃).

In certain embodiments, R^{si} and R^{sj} are taken together with the intervening atom to form an optionally substituted heterocyclic ring of Formula (S-iii-a):

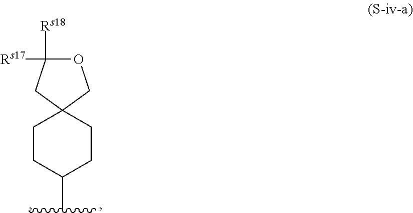

wherein each of R^{s17} and R^{s18} is independently optionally substituted C₁₋₆ alkyl.

In certain embodiments, R^{s17} and R^{s18} are the same. In certain embodiments, R^{s17} and R^{s18} are different. In certain embodiments, R^{s17} and R^{s18} are unsubstituted C₁₋₆ alkyl. In certain embodiments, R^{s17} and R^{s18} are methyl.

In certain embodiments, R^{W1} is of one of the following formulae:

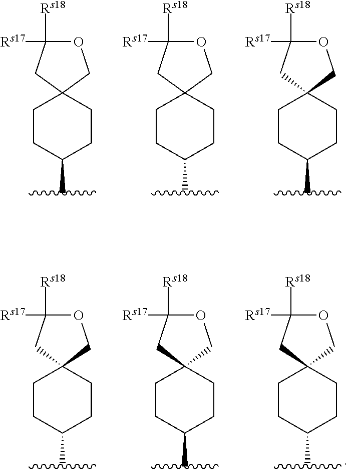

In certain embodiments, R^{si} and R^{sj} are taken together with the intervening atom to form a heterocyclic ring of Formula (S-iii-b):

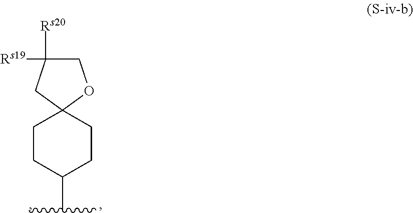

wherein each of R^{s19} and R^{s20} is independently hydrogen or optionally substituted C₁₋₆ alkyl.

In certain embodiments, R^{s19} and R^{s20} are the same. In certain embodiments, R^{s19} and R^{s20} are different. In certain embodiments, R^{s19} and R^{s20} are hydrogen. In certain embodiments, R^{s19} and R^{s20} are substituted C₁₋₆ alkyl. In certain embodiments, R^{s19} and R^{s20} are unsubstituted C₁₋₆ alkyl. In certain embodiments, R^{s19} and R^{s20} are methyl. In certain embodiments, R^{s19} is hydrogen and R^{s20} is optionally substituted C₁₋₆ alkyl. In certain embodiments, R^{s19} is hydrogen and R^{s20} is substituted C₁₋₆ alkyl. In certain embodiments, R^{s19} is hydrogen and R^{s20} is unsubstituted C₁₋₆ alkyl (e.g. methyl or ethyl).

In certain embodiments, R^{W1} is of one of the following formulae:

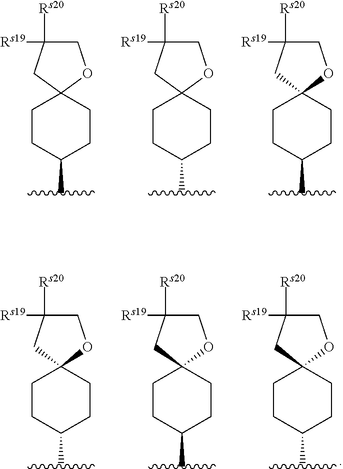

In certain embodiments, a provided compound is not a compound, or pharmaceutically acceptable salt thereof, as provided in PCT/US2014/029710, incorporated herein by reference. In certain embodiments, a provided compound is not a compound, or pharmaceutically acceptable salt thereof, as listed in Table 1.

TABLE 1
Excluded Compounds

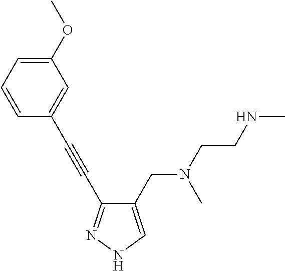 [SMILES: CNCCN(C)Cc1c[nH]nc1C#Cc1cccc(OC)c1]

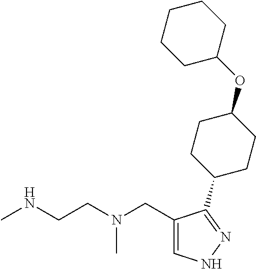 [SMILES: CNCCN(C)Cc1c[nH]nc1[C@H]1CC[C@H](OC2CCCCC2)CC1]

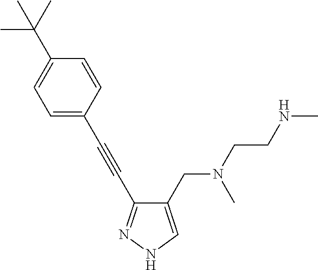 [SMILES: CNCCN(C)Cc1c[nH]nc1C#Cc1ccc(C(C)(C)C)cc1]

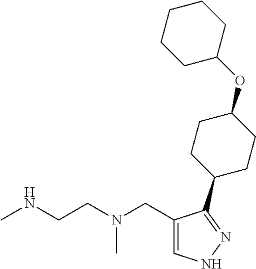 [SMILES: CNCCN(C)Cc1c[nH]nc1[C@H]1CC[C@@H](OC2CCCCC2)CC1]

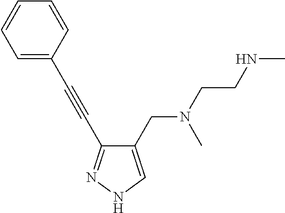 [SMILES: CNCCN(C)Cc1c[nH]nc1C#Cc1ccccc1]

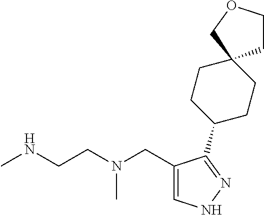 [SMILES: CNCCN(C)Cc1c[nH]nc1[C@H]1CC[C@@]2(CCOC2)CC1]

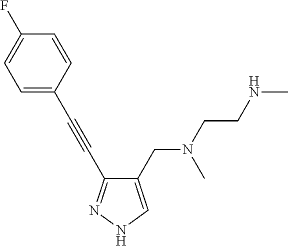 [SMILES: CNCCN(C)Cc1c[nH]nc1C#Cc1ccc(F)cc1]

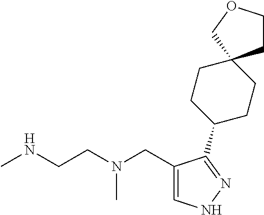 [SMILES: CNCCN(C)Cc1c[nH]nc1[C@H]1CC[C@]2(CCOC2)CC1]

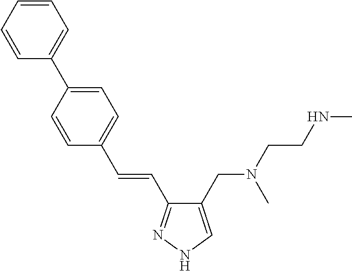 [SMILES: CNCCN(C)Cc1c[nH]nc1/C=C/c1ccc(-c2ccccc2)cc1]

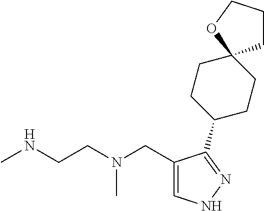 [SMILES: CNCCN(C)Cc1c[nH]nc1[C@H]1CC[C@@]2(CCCO2)CC1]

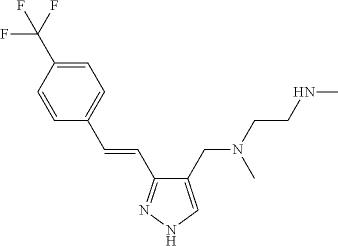 [SMILES: CNCCN(C)Cc1c[nH]nc1/C=C/c1ccc(C(F)(F)F)cc1]

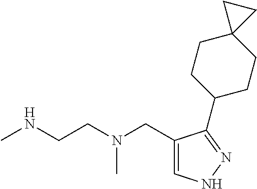 [SMILES: CNCCN(C)Cc1c[nH]nc1C1CCC2(CC1)CC2]

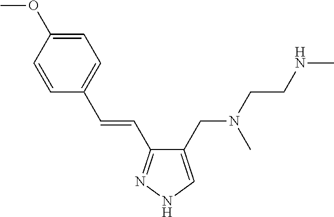

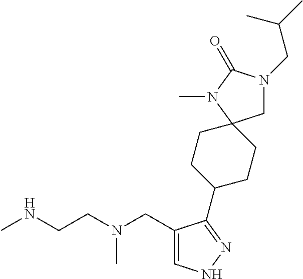 [SMILES: CNCCN(C)Cc1c[nH]nc1C1CCC2(CC1)CN(CC(C)C)C(=O)N2C]

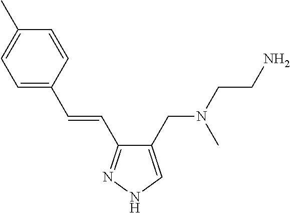 [SMILES: Cc1ccc(/C=C/c2n[nH]cc2CN(C)CCN)cc1]

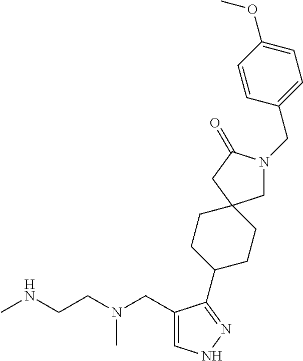 [SMILES: CNCCN(C)Cc1c[nH]nc1C1CCC2(CC1)CC(=O)N(Cc1ccc(OC)cc1)C2]

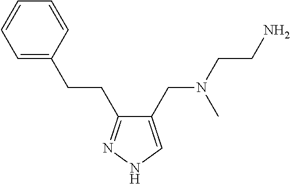 [SMILES: CN(CCN)Cc1c[nH]nc1CCc1ccccc1]

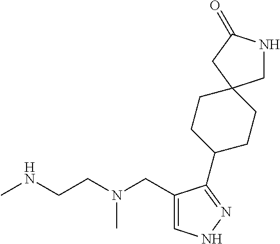 [SMILES: CNCCN(C)Cc1c[nH]nc1C1CCC2(CC1)CNC(=O)C2]

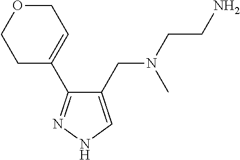 [SMILES: CN(CCN)Cc1c[nH]nc1C1=CCOCC1]

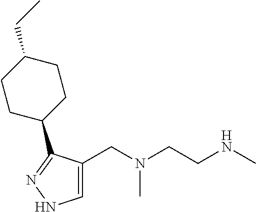 [SMILES: CC[C@H]1CC[C@H](c2n[nH]cc2CN(C)CCNC)CC1]

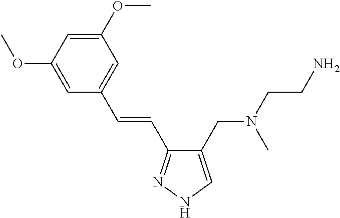 [SMILES: COc1cc(/C=C/c2n[nH]cc2CN(C)CCN)cc(OC)c1]

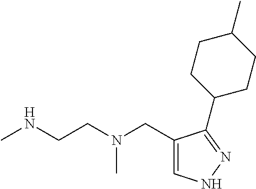 [SMILES: CNCCN(C)Cc1c[nH]nc1C1CCC(C)CC1]

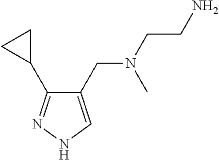

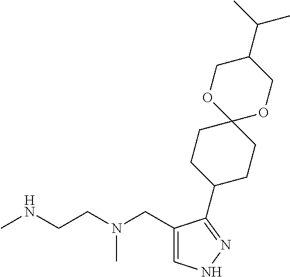 [SMILES: CNCCN(C)Cc1c[nH]nc1C1CCC2(CC1)OCC(C(C)C)CO2]

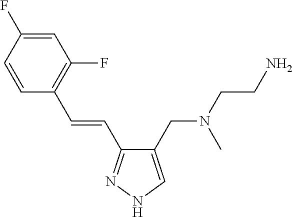 [SMILES: CN(CCN)Cc1c[nH]nc1/C=C/c1ccc(F)cc1F]

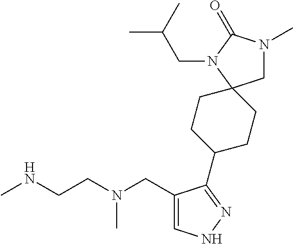

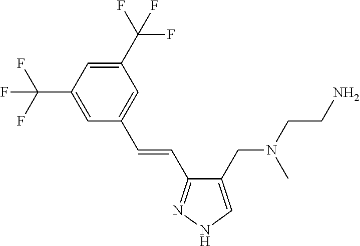 [SMILES: CN(CCN)Cc1c[nH]nc1/C=C/c1cc(C(F)(F)F)cc(C(F)(F)F)c1]

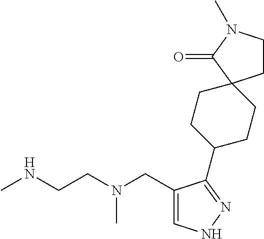 [SMILES: CNCCN(C)Cc1c[nH]nc1C1CCC2(CC1)CCN(C)C2=O]

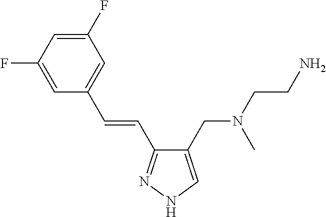 [SMILES: CN(CCN)Cc1c[nH]nc1/C=C/c1cc(F)cc(F)c1]

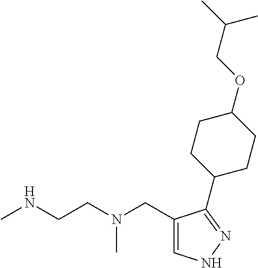 [SMILES: CNCCN(C)Cc1c[nH]nc1C1CCC(OCC(C)C)CC1]

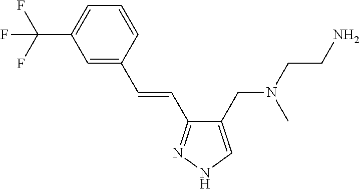 [SMILES: CN(CCN)Cc1c[nH]nc1/C=C/c1cccc(C(F)(F)F)c1]

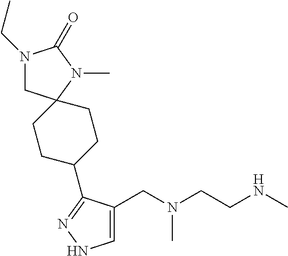 [SMILES: CCN1CC2(CCC(c3n[nH]cc3CN(C)CCNC)CC2)N(C)C1=O]

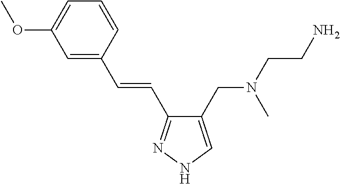 [SMILES: COc1cccc(/C=C/c2n[nH]cc2CN(C)CCN)c1]

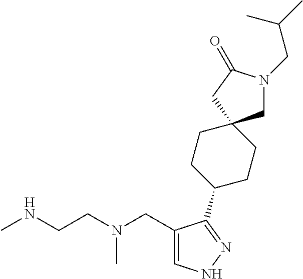 [SMILES: CNCCN(C)Cc1c[nH]nc1[C@H]1CC[C@]2(CC1)CC(=O)N(CC(C)C)C2]

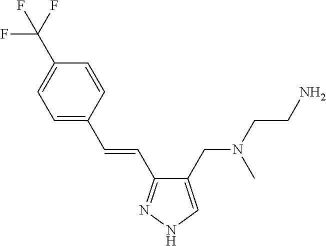 [SMILES: CN(CCN)Cc1c[nH]nc1/C=C/c1ccc(C(F)(F)F)cc1]

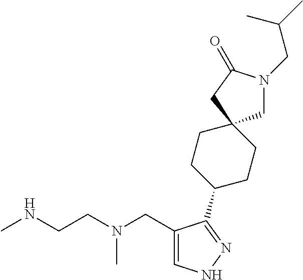 [SMILES: CNCCN(C)Cc1c[nH]nc1[C@H]1CC[C@@]2(CC1)CC(=O)N(CC(C)C)C2]

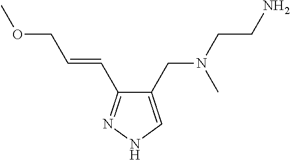 [SMILES: COC/C=C/c1n[nH]cc1CN(C)CCN]

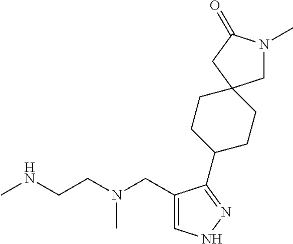

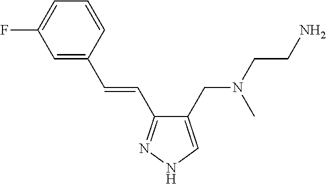 [SMILES: CN(CCN)Cc1c[nH]nc1/C=C/c1cccc(F)c1]

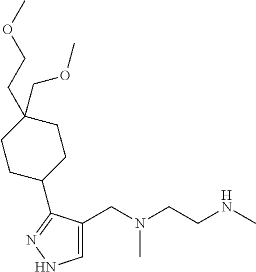 [SMILES: CNCCN(C)Cc1c[nH]nc1C1CCC(CCOC)(COC)CC1]

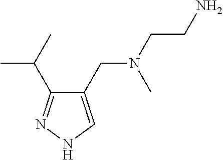

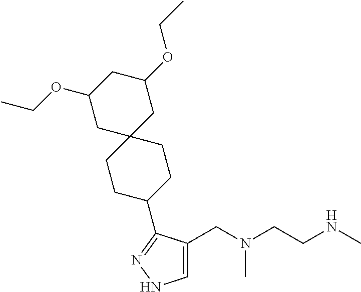 [SMILES: CCOC1CC(OCC)CC2(CCC(c3n[nH]cc3CN(C)CCNC)CC2)C1]

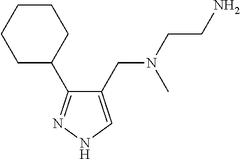 [SMILES: CN(CCN)Cc1c[nH]nc1C1CCCCC1]

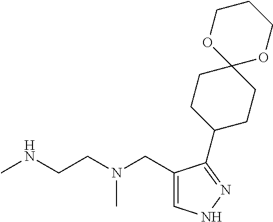 [SMILES: CNCCN(C)Cc1c[nH]nc1C1CCC2(CC1)OCCCO2]

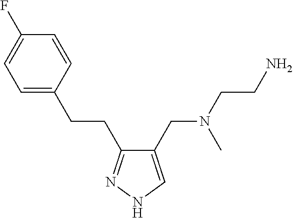 [SMILES: CN(CCN)Cc1c[nH]nc1CCc1ccc(F)cc1]

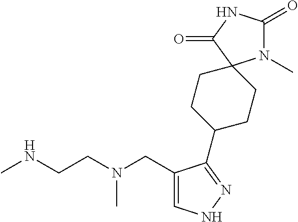 [SMILES: CNCCN(C)Cc1c[nH]nc1C1CCC2(CC1)C(=O)NC(=O)N2C]

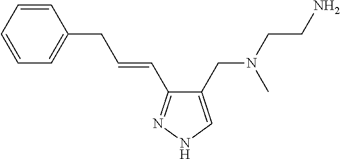 [SMILES: CN(CCN)Cc1c[nH]nc1/C=C/Cc1ccccc1]

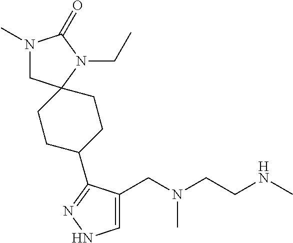

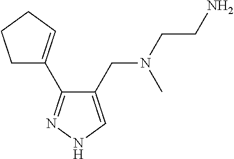 [SMILES: CN(CCN)Cc1c[nH]nc1C1=CCCC1]

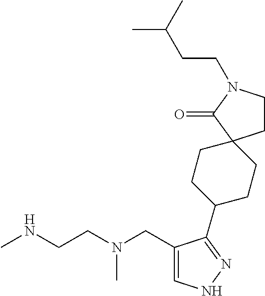 [SMILES: CNCCN(C)Cc1c[nH]nc1C1CCC2(CC1)CCN(CCC(C)C)C2=O]

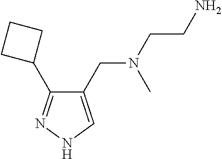

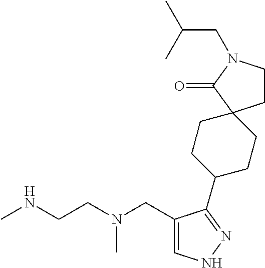 [SMILES: CNCCN(C)Cc1c[nH]nc1C1CCC2(CC1)CCN(CC(C)C)C2=O]

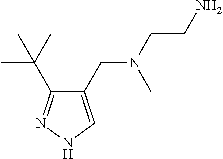

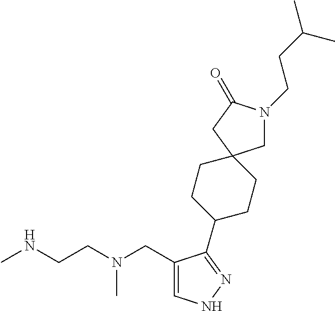 [SMILES: CNCCN(C)Cc1c[nH]nc1C1CCC2(CC1)CC(=O)N(CCC(C)C)C2]

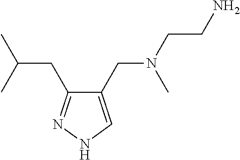

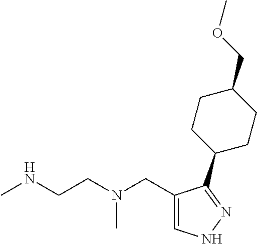 [SMILES: CNCCN(C)Cc1c[nH]nc1[C@H]1CC[C@@H](COC)CC1]

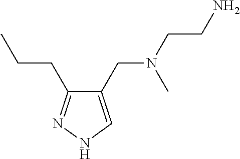

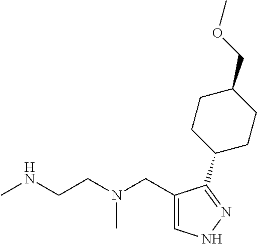 [SMILES: CNCCN(C)Cc1c[nH]nc1[C@H]1CC[C@H](COC)CC1]

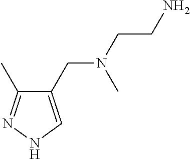

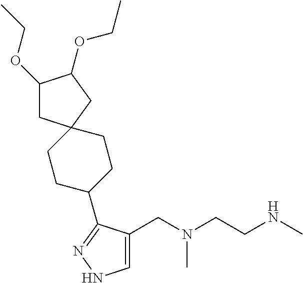 [SMILES: CCOC1CC2(CCC(c3n[nH]cc3CN(C)CCNC)CC2)CC1OCC]

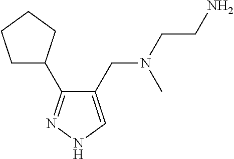 [SMILES: CN(CCN)Cc1c[nH]nc1C1CCCC1]

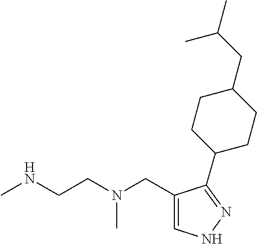 [SMILES: CNCCN(C)Cc1c[nH]nc1C1CCC(CC(C)C)CC1]

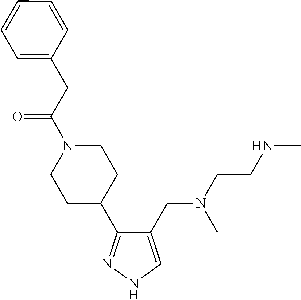 [SMILES: CNCCN(C)Cc1c[nH]nc1C1CCN(C(=O)Cc2ccccc2)CC1]

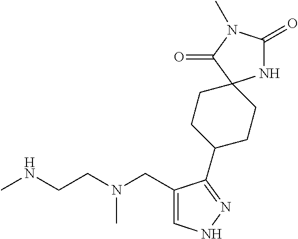 [SMILES: CNCCN(C)Cc1c[nH]nc1C1CCC2(CC1)NC(=O)N(C)C2=O]

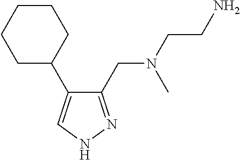 [SMILES: CN(CCN)Cc1n[nH]cc1C1CCCCC1]

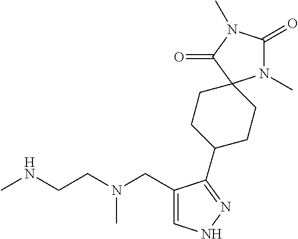 [SMILES: CNCCN(C)Cc1c[nH]nc1C1CCC2(CC1)C(=O)N(C)C(=O)N2C]

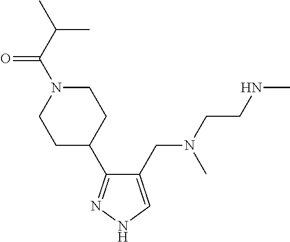 [SMILES: CNCCN(C)Cc1c[nH]nc1C1CCN(C(=O)C(C)C)CC1]

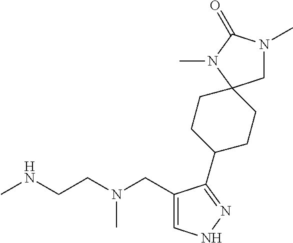

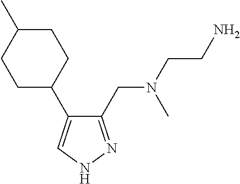 [SMILES: CC1CCC(c2c[nH]nc2CN(C)CCN)CC1]

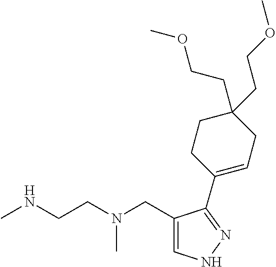 [SMILES: CNCCN(C)Cc1c[nH]nc1C1=CCC(CCOC)(CCOC)CC1]

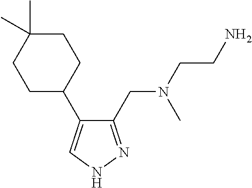 [SMILES: CN(CCN)Cc1n[nH]cc1C1CCC(C)(C)CC1]

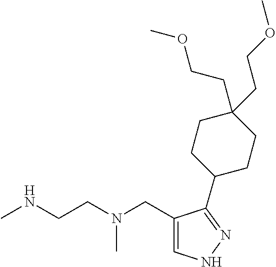 [SMILES: CNCCN(C)Cc1c[nH]nc1C1CCC(CCOC)(CCOC)CC1]

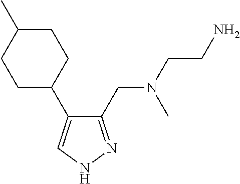 [SMILES: CC1CCC(c2c[nH]nc2CN(C)CCN)CC1]

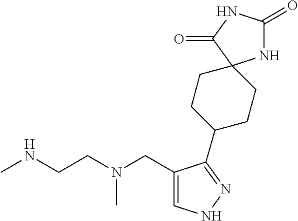 [SMILES: CNCCN(C)Cc1c[nH]nc1C1CCC2(CC1)NC(=O)NC2=O]

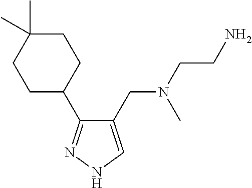 [SMILES: CN(CCN)Cc1c[nH]nc1C1CCC(C)(C)CC1]

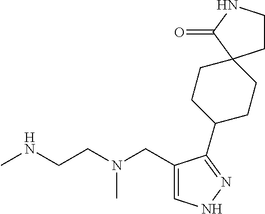 [SMILES: CNCCN(C)Cc1c[nH]nc1C1CCC2(CCNC2=O)CC1]

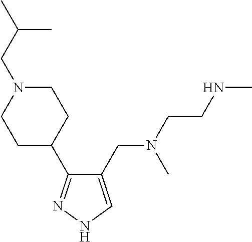 [SMILES: CNCCN(C)Cc1c[nH]nc1C1CCN(CC(C)C)CC1]

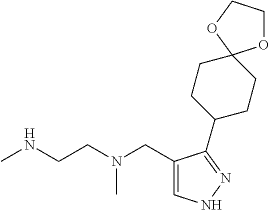 [SMILES: CNCCN(C)Cc1c[nH]nc1C1CCC2(CC1)OCCO2]

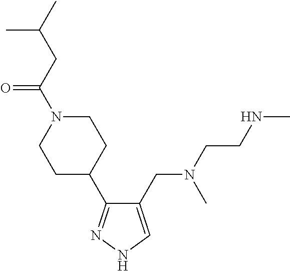 [SMILES: CNCCN(C)Cc1c[nH]nc1C1CCN(C(=O)CC(C)C)CC1]

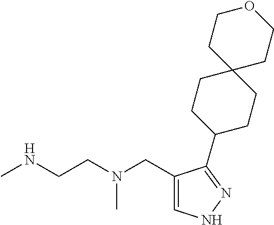 [SMILES: CNCCN(C)Cc1c[nH]nc1C1CCC2(CCOCC2)CC1]

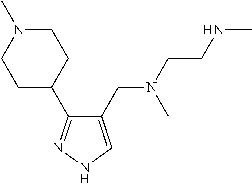 [SMILES: CNCCN(C)Cc1c[nH]nc1C1CCN(C)CC1]

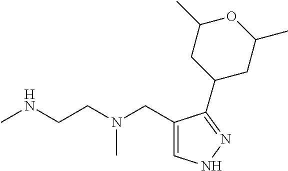

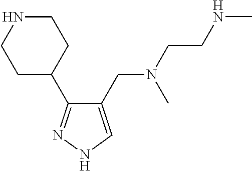 [SMILES: CNCCN(C)Cc1c[nH]nc1C1CCNCC1]

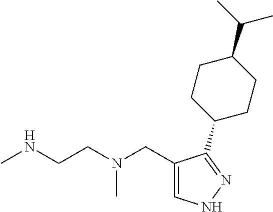 [SMILES: CNCCN(C)Cc1c[nH]nc1[C@H]1CC[C@H](C(C)C)CC1]

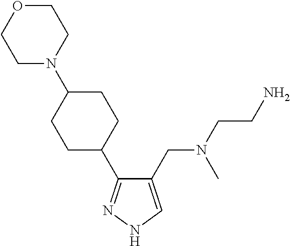 [SMILES: CN(CCN)Cc1c[nH]nc1C1CCC(N2CCOCC2)CC1]

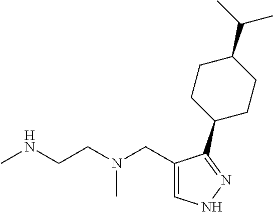 [SMILES: CNCCN(C)Cc1c[nH]nc1[C@H]1CC[C@@H](C(C)C)CC1]

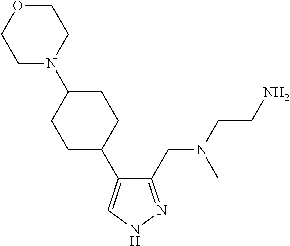 [SMILES: CN(CCN)Cc1n[nH]cc1C1CCC(N2CCOCC2)CC1]

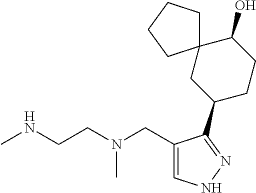 [SMILES: CNCCN(C)Cc1c[nH]nc1[C@@H]1CC[C@H](O)C2(CCCC2)C1]

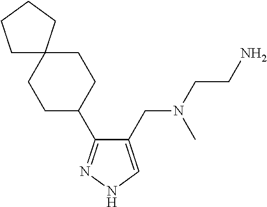 [SMILES: CN(CCN)Cc1c[nH]nc1C1CCC2(CCCC2)CC1]

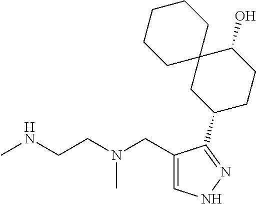 [SMILES: CNCCN(C)Cc1c[nH]nc1[C@H]1CC[C@@H](O)C2(CCCCC2)C1]

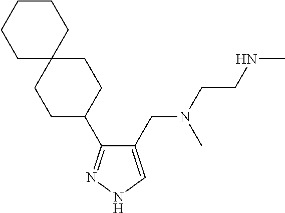 [SMILES: CNCCN(C)Cc1c[nH]nc1C1CCC2(CCCCC2)CC1]

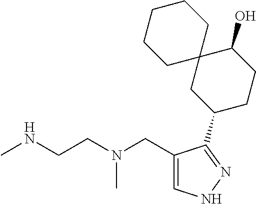 [SMILES: CNCCN(C)Cc1c[nH]nc1[C@H]1CC[C@H](O)C2(CCCCC2)C1]

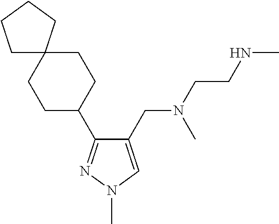 [SMILES: CNCCN(C)Cc1cn(C)nc1C1CCC2(CCCC2)CC1]

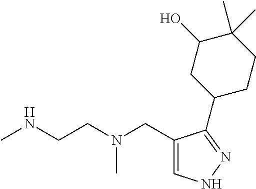

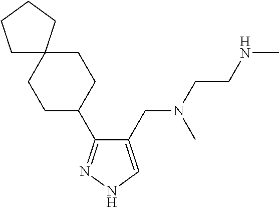 [SMILES: CNCCN(C)Cc1c[nH]nc1C1CCC2(CCCC2)CC1]

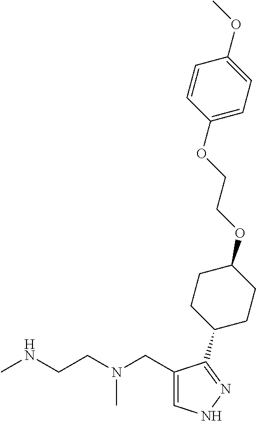 [SMILES: CNCCN(C)Cc1c[nH]nc1[C@H]1CC[C@H](OCCOc2ccc(OC)cc2)CC1]

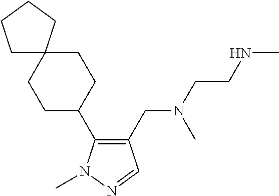 [SMILES: CNCCN(C)Cc1cnn(C)c1C1CCC2(CCCC2)CC1]

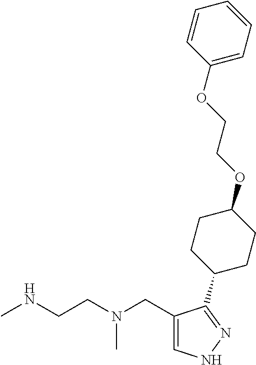 [SMILES: CNCCN(C)Cc1c[nH]nc1[C@H]1CC[C@H](OCCOc2ccccc2)CC1]

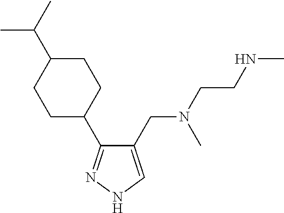 [SMILES: CNCCN(C)Cc1c[nH]nc1C1CCC(C(C)C)CC1]

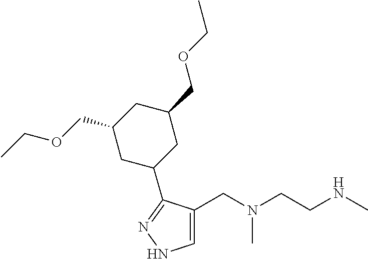 [SMILES: CCOC[C@H]1CC(c2n[nH]cc2CN(C)CCNC)C[C@H](COCC)C1]

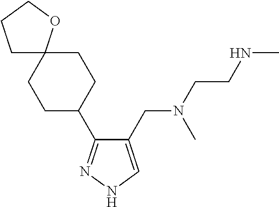 [SMILES: CNCCN(C)Cc1c[nH]nc1C1CCC2(CCCO2)CC1]

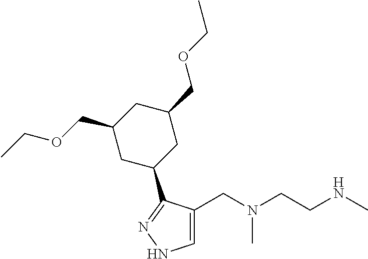 [SMILES: CCOC[C@@H]1C[C@H](COCC)C[C@H](c2n[nH]cc2CN(C)CCNC)C1]

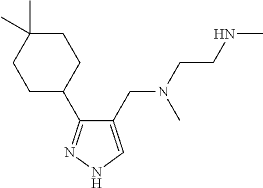

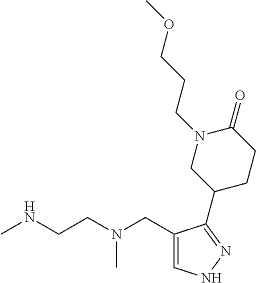 [SMILES: CNCCN(C)Cc1c[nH]nc1C1CCC(=O)N(CCCOC)C1]

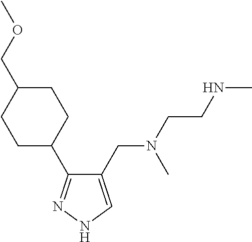

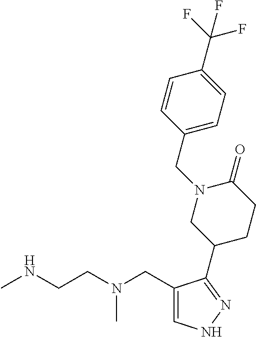 [SMILES: CNCCN(C)Cc1c[nH]nc1C1CCC(=O)N(Cc2ccc(C(F)(F)F)cc2)C1]

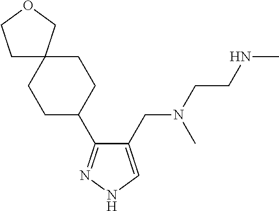 [SMILES: CNCCN(C)Cc1c[nH]nc1C1CCC2(CCOC2)CC1]

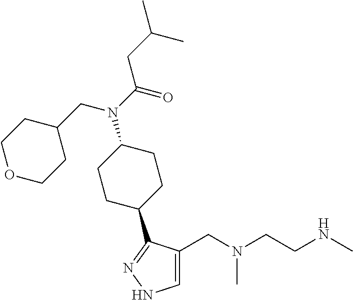 [SMILES: CNCCN(C)Cc1c[nH]nc1[C@H]1CC[C@H](N(CC2CCOCC2)C(=O)CC(C)C)CC1]

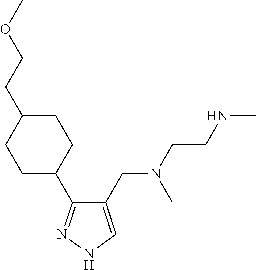 [SMILES: CNCCN(C)Cc1c[nH]nc1C1CCC(CCOC)CC1]

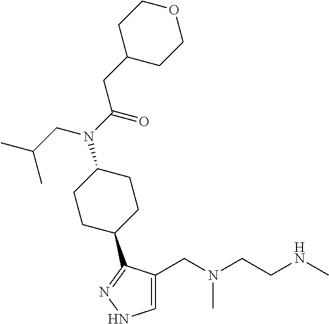 [SMILES: CNCCN(C)Cc1c[nH]nc1[C@H]1CC[C@H](N(CC(C)C)C(=O)CC2CCOCC2)CC1]

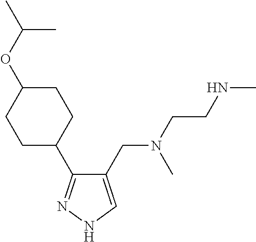 [SMILES: CNCCN(C)Cc1c[nH]nc1C1CCC(OC(C)C)CC1]

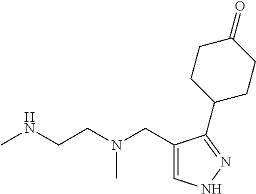 [SMILES: CNCCN(C)Cc1c[nH]nc1C1CCC(=O)CC1]

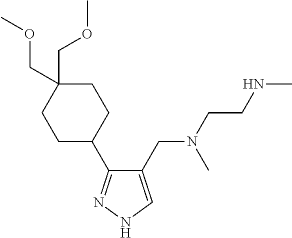

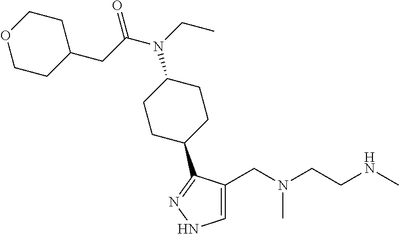 [SMILES: CCN(C(=O)CC1CCOCC1)[C@H]1CC[C@H](c2n[nH]cc2CN(C)CCNC)CC1]

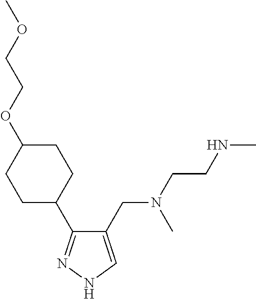 [SMILES: CNCCN(C)Cc1c[nH]nc1C1CCC(OCCOC)CC1]

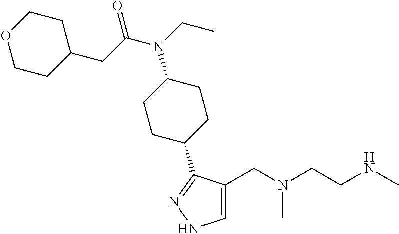 [SMILES: CCN(C(=O)CC1CCOCC1)[C@H]1CC[C@@H](c2n[nH]cc2CN(C)CCNC)CC1]

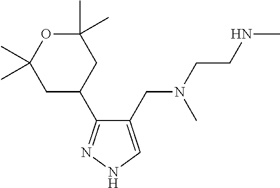

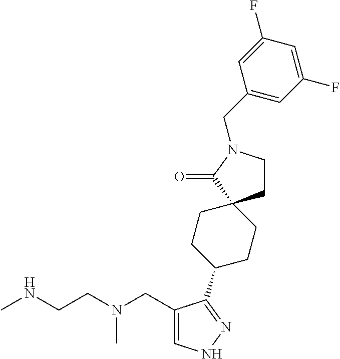 [SMILES: CNCCN(C)Cc1c[nH]nc1[C@H]1CC[C@]2(CCN(Cc3cc(F)cc(F)c3)C2=O)CC1]

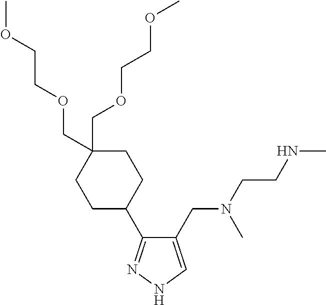 [SMILES: CNCCN(C)Cc1c[nH]nc1C1CCC(COCCOC)(COCCOC)CC1]

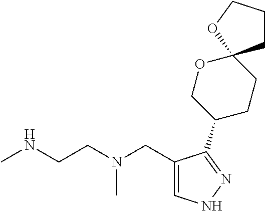 [SMILES: CNCCN(C)Cc1c[nH]nc1[C@H]1CC[C@]2(CCCO2)OC1]

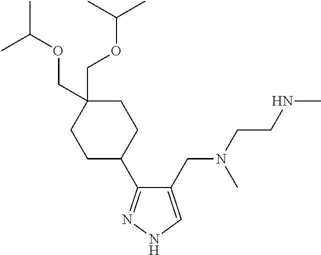 [SMILES: CNCCN(C)Cc1c[nH]nc1C1CCC(COC(C)C)(COC(C)C)CC1]

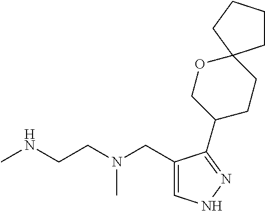 [SMILES: CNCCN(C)Cc1c[nH]nc1C1CCC2(CCCC2)OC1]

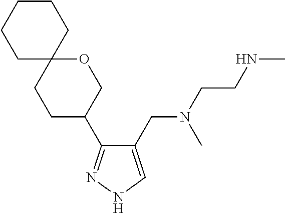 [SMILES: CNCCN(C)Cc1c[nH]nc1C1CCC2(CCCCC2)OC1]

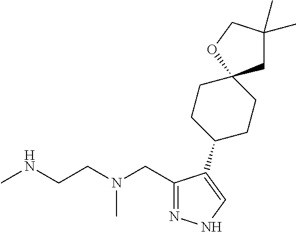

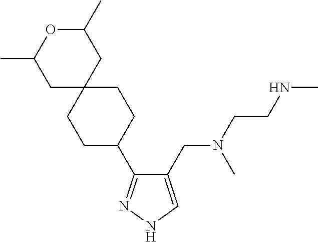

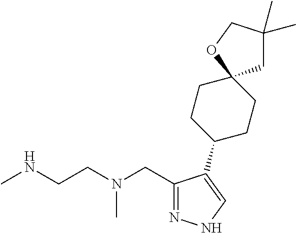

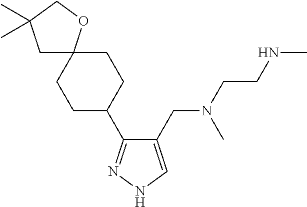

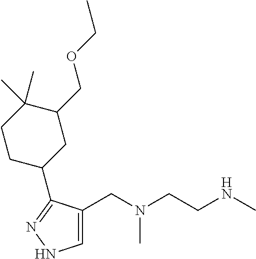 [SMILES: CCOCC1CC(c2n[nH]cc2CN(C)CCNC)CCC1(C)C]

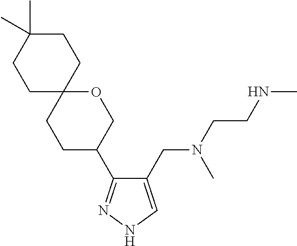

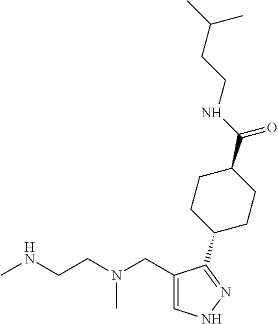 [SMILES: CNCCN(C)Cc1c[nH]nc1[C@H]1CC[C@H](C(=O)NCCC(C)C)CC1]

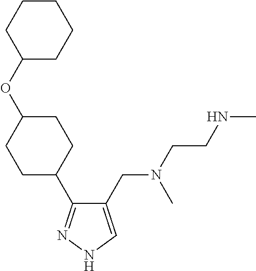 [SMILES: CNCCN(C)Cc1c[nH]nc1C1CCC(OC2CCCCC2)CC1]

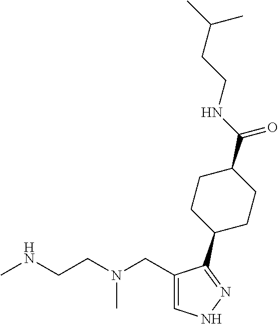 [SMILES: CNCCN(C)Cc1c[nH]nc1[C@H]1CC[C@@H](C(=O)NCCC(C)C)CC1]

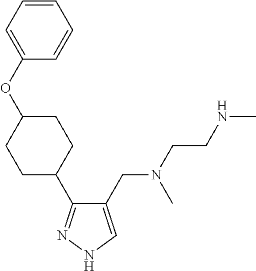 [SMILES: CNCCN(C)Cc1c[nH]nc1C1CCC(Oc2ccccc2)CC1]

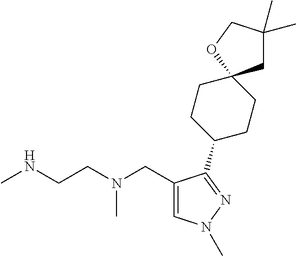

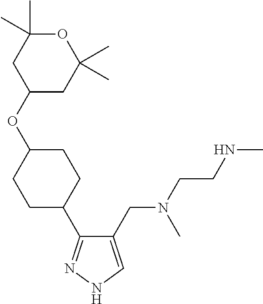

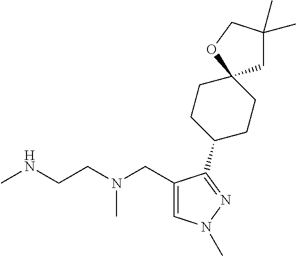

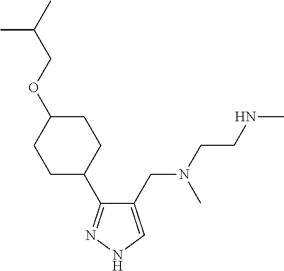 [SMILES: CNCCN(C)Cc1c[nH]nc1C1CCC(OCC(C)C)CC1]

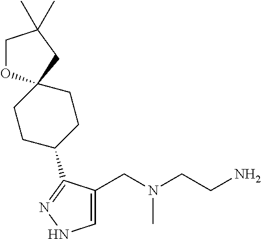 [SMILES: CN(CCN)Cc1c[nH]nc1[C@H]1CC[C@@]2(CC1)CC(C)(C)CO2]

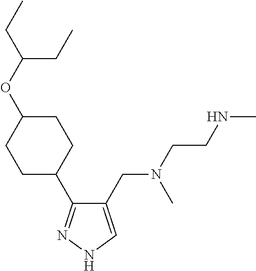 [SMILES: CCC(CC)OC1CCC(c2n[nH]cc2CN(C)CCNC)CC1]

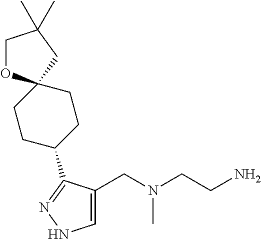 [SMILES: CN(CCN)Cc1c[nH]nc1[C@H]1CC[C@]2(CC1)CC(C)(C)CO2]

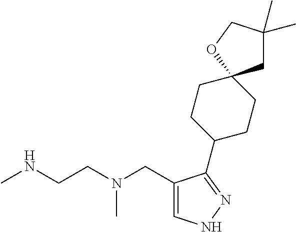

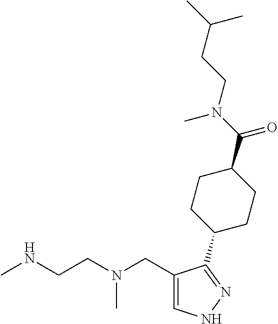 [SMILES: CNCCN(C)Cc1c[nH]nc1[C@H]1CC[C@H](C(=O)N(C)CCC(C)C)CC1]

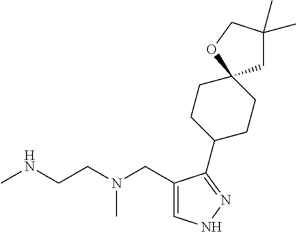

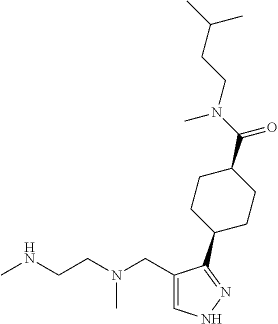 [SMILES: CNCCN(C)Cc1c[nH]nc1[C@H]1CC[C@@H](C(=O)N(C)CCC(C)C)CC1]

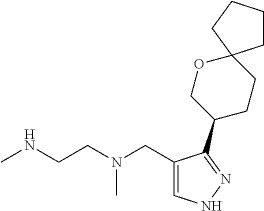 [SMILES: CNCCN(C)Cc1c[nH]nc1[C@@H]1CCC2(CCCC2)OC1]

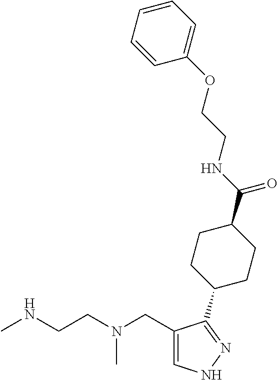 [SMILES: CNCCN(C)Cc1c[nH]nc1[C@H]1CC[C@H](C(=O)NCCOc2ccccc2)CC1]

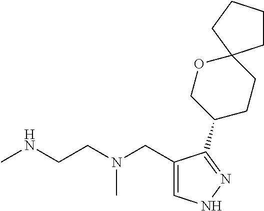 [SMILES: CNCCN(C)Cc1c[nH]nc1[C@H]1CCC2(CCCC2)OC1]

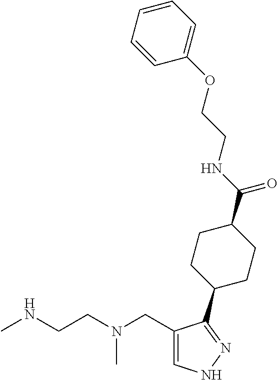 [SMILES: CNCCN(C)Cc1c[nH]nc1[C@H]1CC[C@@H](C(=O)NCCOc2ccccc2)CC1]

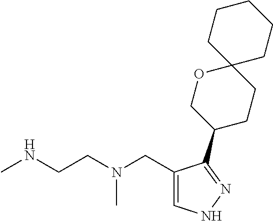 [SMILES: CNCCN(C)Cc1c[nH]nc1[C@@H]1CCC2(CCCCC2)OC1]

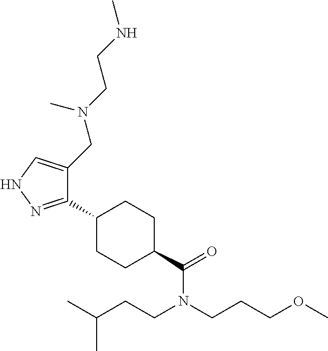 [SMILES: CNCCN(C)Cc1c[nH]nc1[C@H]1CC[C@H](C(=O)N(CCCOC)CCC(C)C)CC1]

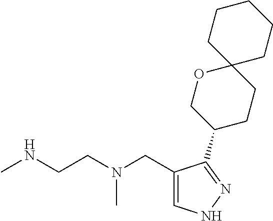 [SMILES: CNCCN(C)Cc1c[nH]nc1[C@H]1CCC2(CCCCC2)OC1]

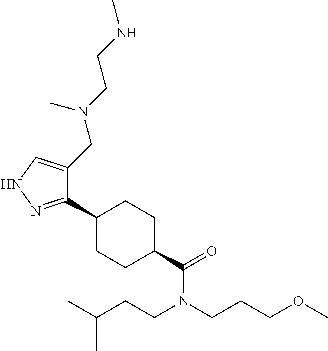 [SMILES: CNCCN(C)Cc1c[nH]nc1[C@H]1CC[C@@H](C(=O)N(CCCOC)CCC(C)C)CC1]

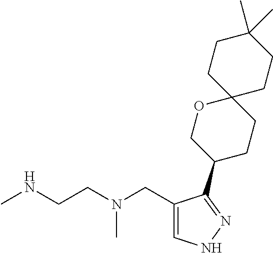

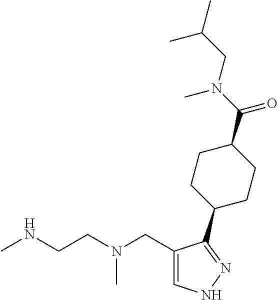 [SMILES: CNCCN(C)Cc1c[nH]nc1[C@H]1CC[C@@H](C(=O)N(C)CC(C)C)CC1]

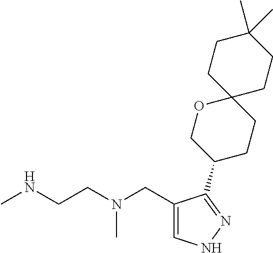

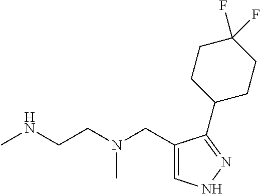 [SMILES: CNCCN(C)Cc1c[nH]nc1C1CCC(F)(F)CC1]

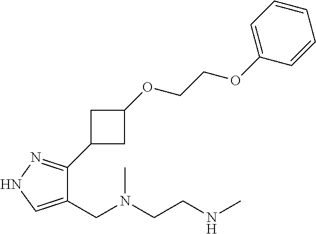 [SMILES: CNCCN(C)Cc1c[nH]nc1C1CC(OCCOc2ccccc2)C1]

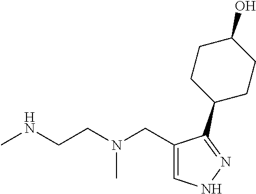 [SMILES: CNCCN(C)Cc1c[nH]nc1[C@H]1CC[C@@H](O)CC1]

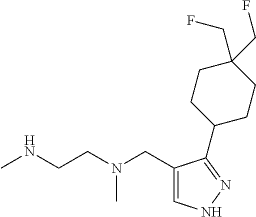 [SMILES: CNCCN(C)Cc1c[nH]nc1C1CCC(CF)(CF)CC1]

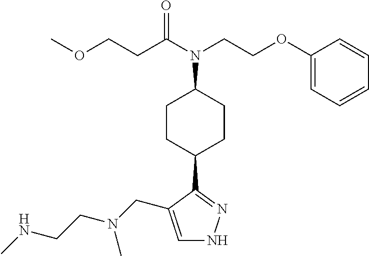 [SMILES: CNCCN(C)Cc1c[nH]nc1[C@H]1CC[C@@H](N(CCOc2ccccc2)C(=O)CCOC)CC1]

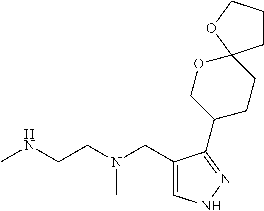 [SMILES: CNCCN(C)Cc1c[nH]nc1C1CCC2(CCCO2)OC1]

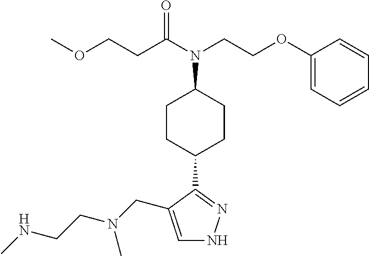 [SMILES: CNCCN(C)Cc1c[nH]nc1[C@H]1CC[C@H](N(CCOc2ccccc2)C(=O)CCOC)CC1]

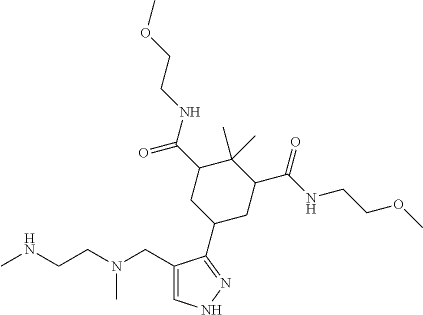

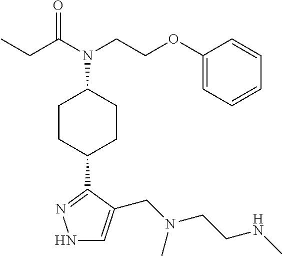 [SMILES: CCC(=O)N(CCOc1ccccc1)[C@H]1CC[C@@H](c2n[nH]cc2CN(C)CCNC)CC1]

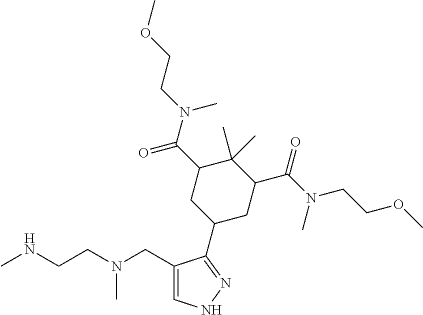

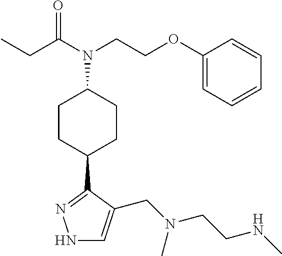 [SMILES: CCC(=O)N(CCOc1ccccc1)[C@H]1CC[C@H](c2n[nH]cc2CN(C)CCNC)CC1]

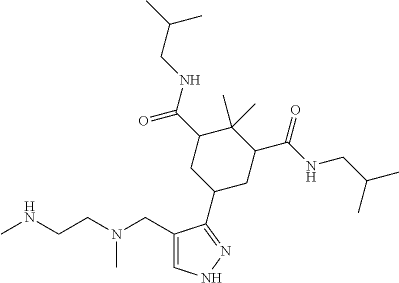 [SMILES: CNCCN(C)Cc1c[nH]nc1C1CC(C(=O)NCC(C)C)C(C)(C)C(C(=O)NCC(C)C)C1]

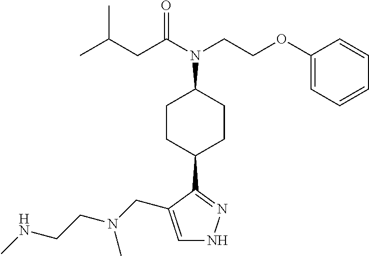 [SMILES: CNCCN(C)Cc1c[nH]nc1[C@H]1CC[C@@H](N(CCOc2ccccc2)C(=O)CC(C)C)CC1]

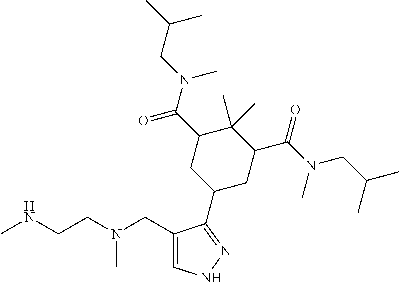 [SMILES: CNCCN(C)Cc1c[nH]nc1C1CC(C(=O)N(C)CC(C)C)C(C)(C)C(C(=O)N(C)CC(C)C)C1]

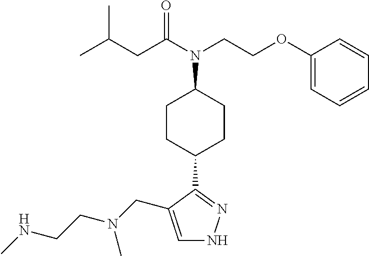 [SMILES: CNCCN(C)Cc1c[nH]nc1[C@H]1CC[C@H](N(CCOc2ccccc2)C(=O)CC(C)C)CC1]

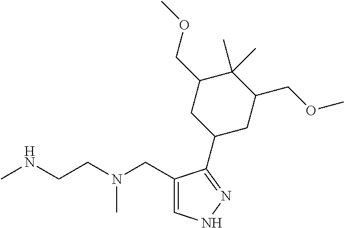

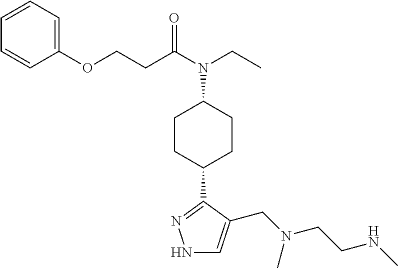 [SMILES: CCN(C(=O)CCOc1ccccc1)[C@H]1CC[C@@H](c2n[nH]cc2CN(C)CCNC)CC1]

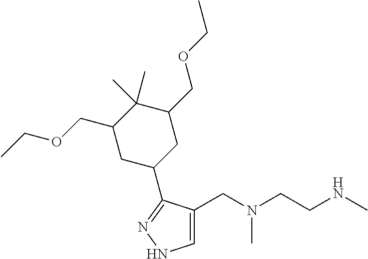 [SMILES: CCOCC1CC(c2n[nH]cc2CN(C)CCNC)CC(COCC)C1(C)C]

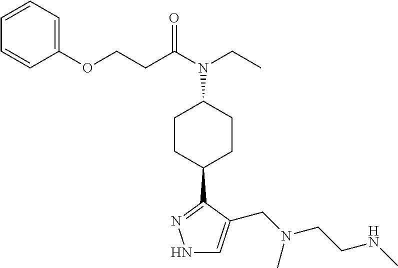 [SMILES: CCN(C(=O)CCOc1ccccc1)[C@H]1CC[C@H](c2n[nH]cc2CN(C)CCNC)CC1]

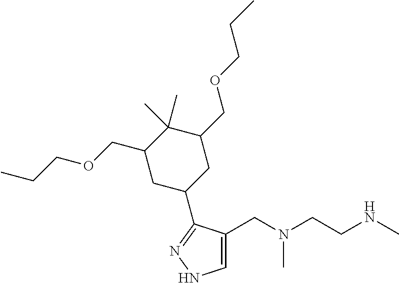 [SMILES: CCCOCC1CC(c2n[nH]cc2CN(C)CCNC)CC(COCCC)C1(C)C]

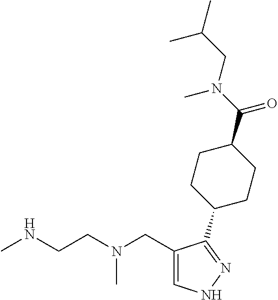 [SMILES: CNCCN(C)Cc1c[nH]nc1[C@H]1CC[C@H](C(=O)N(C)CC(C)C)CC1]

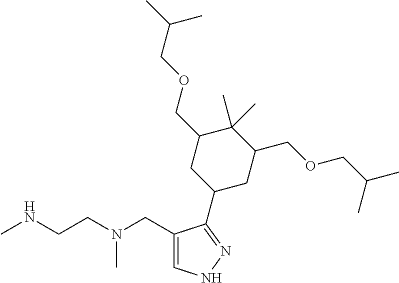 [SMILES: CNCCN(C)Cc1c[nH]nc1C1CC(COCC(C)C)C(C)(C)C(COCC(C)C)C1]

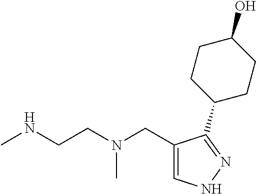 [SMILES: CNCCN(C)Cc1c[nH]nc1[C@H]1CC[C@H](O)CC1]

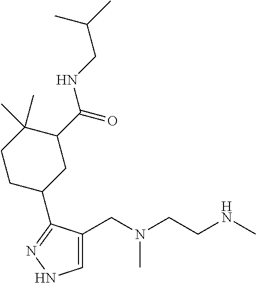 [SMILES: CNCCN(C)Cc1c[nH]nc1C1CCC(C)(C)C(C(=O)NCC(C)C)C1]

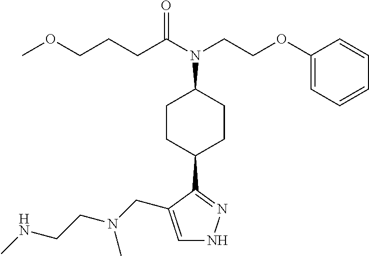 [SMILES: CNCCN(C)Cc1c[nH]nc1[C@H]1CC[C@@H](N(CCOc2ccccc2)C(=O)CCCOC)CC1]

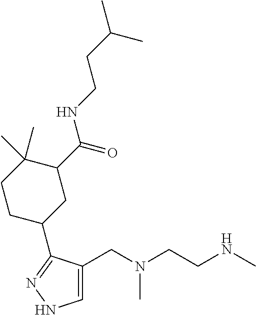 [SMILES: CNCCN(C)Cc1c[nH]nc1C1CCC(C)(C)C(C(=O)NCCC(C)C)C1]

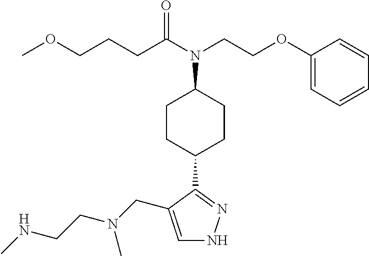 [SMILES: CNCCN(C)Cc1c[nH]nc1[C@H]1CC[C@H](N(CCOc2ccccc2)C(=O)CCCOC)CC1]

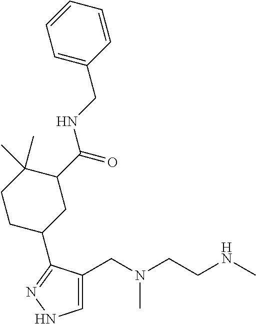 [SMILES: CNCCN(C)Cc1c[nH]nc1C1CCC(C)(C)C(C(=O)NCc2ccccc2)C1]

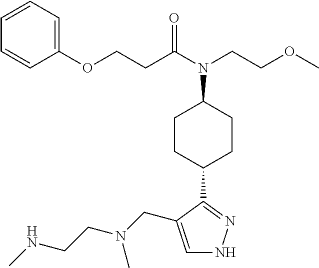 [SMILES: CNCCN(C)Cc1c[nH]nc1[C@H]1CC[C@H](N(CCOC)C(=O)CCOc2ccccc2)CC1]

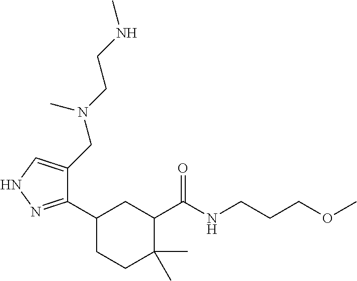 [SMILES: CNCCN(C)Cc1c[nH]nc1C1CCC(C)(C)C(C(=O)NCCCOC)C1]

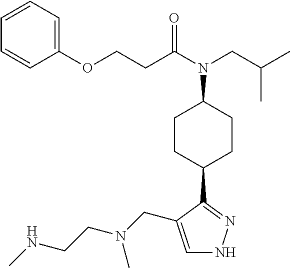 [SMILES: CNCCN(C)Cc1c[nH]nc1[C@H]1CC[C@@H](N(CC(C)C)C(=O)CCOc2ccccc2)CC1]

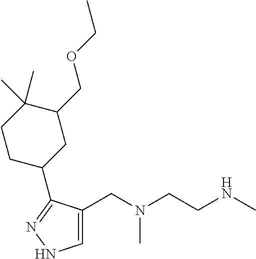 [SMILES: CCOCC1CC(c2n[nH]cc2CN(C)CCNC)CCC1(C)C]

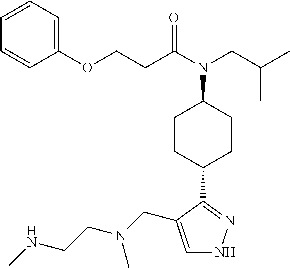 [SMILES: CNCCN(C)Cc1c[nH]nc1[C@H]1CC[C@H](N(CC(C)C)C(=O)CCOc2ccccc2)CC1]

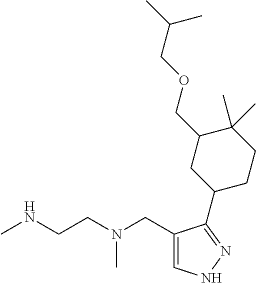

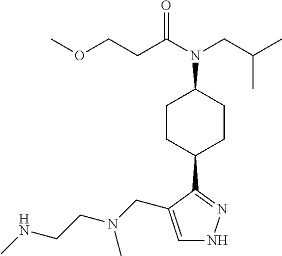 [SMILES: CNCCN(C)Cc1c[nH]nc1[C@H]1CC[C@@H](N(CC(C)C)C(=O)CCOC)CC1]

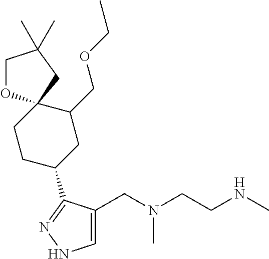 [SMILES: CCOCC1C[C@H](c2n[nH]cc2CN(C)CCNC)CC[C@@]12CC(C)(C)CO2]

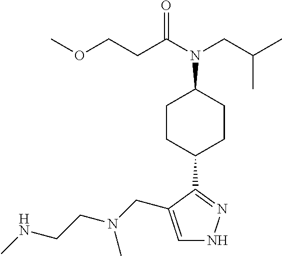 [SMILES: CNCCN(C)Cc1c[nH]nc1[C@H]1CC[C@H](N(CC(C)C)C(=O)CCOC)CC1]

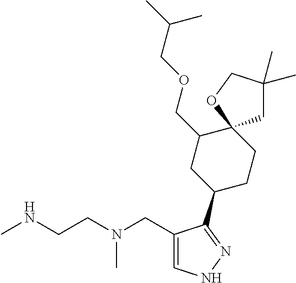

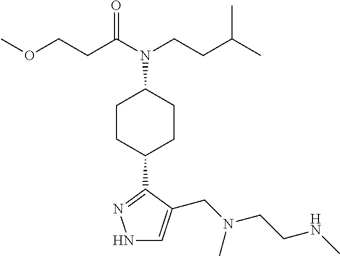

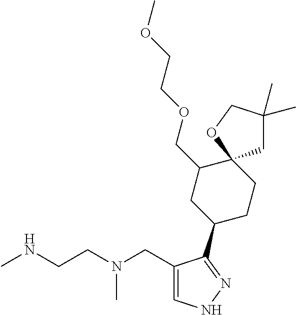

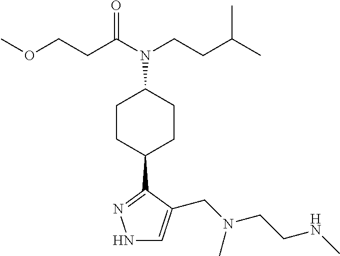

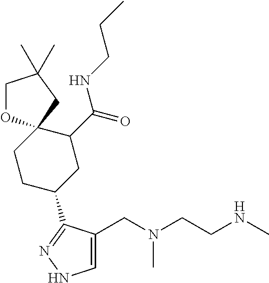 [SMILES: CCCNC(=O)C1C[C@H](c2n[nH]cc2CN(C)CCNC)CC[C@@]12CC(C)(C)CO2]

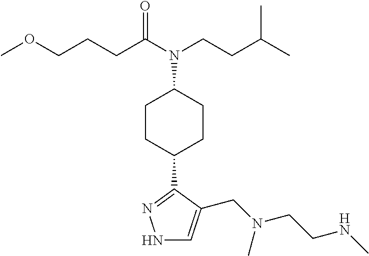

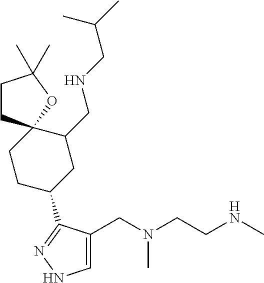 [SMILES: CNCCN(C)Cc1c[nH]nc1[C@@H]1CC[C@@]2(CCC(C)(C)O2)C(CNCC(C)C)C1]

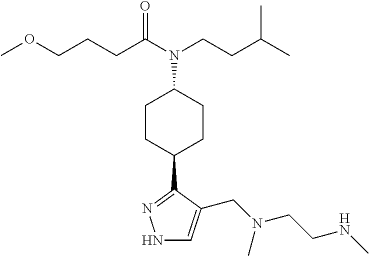

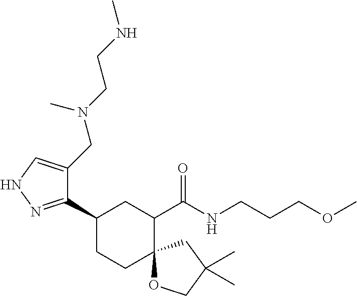 [SMILES: CNCCN(C)Cc1c[nH]nc1[C@@H]1CC[C@@]2(CC(C)(C)CO2)C(C(=O)NCCCOC)C1]

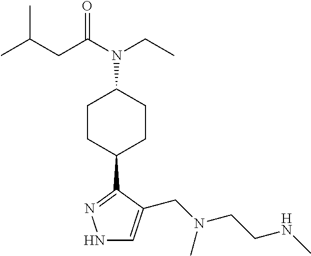 [SMILES: CCN(C(=O)CC(C)C)[C@H]1CC[C@H](c2n[nH]cc2CN(C)CCNC)CC1]

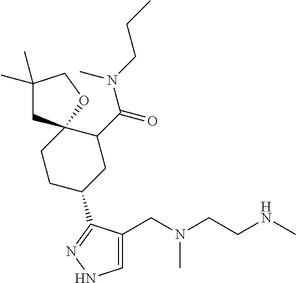

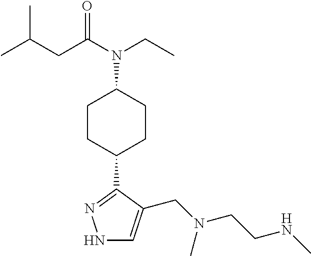 [SMILES: CCN(C(=O)CC(C)C)[C@H]1CC[C@@H](c2n[nH]cc2CN(C)CCNC)CC1]

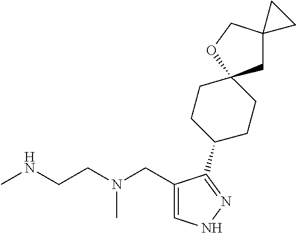 [SMILES: CNCCN(C)Cc1c[nH]nc1[C@H]1CC[C@@]2(CC1)CC1(CC1)CO2]

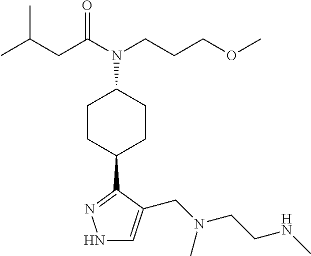 [SMILES: CNCCN(C)Cc1c[nH]nc1[C@H]1CC[C@H](N(CCCOC)C(=O)CC(C)C)CC1]

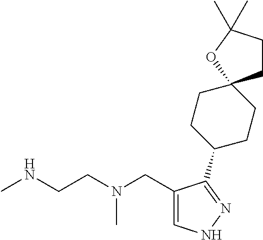

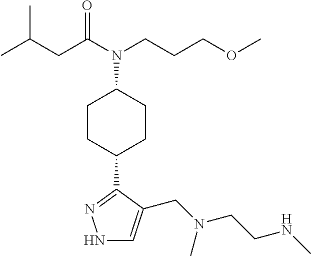 [SMILES: CNCCN(C)Cc1c[nH]nc1[C@H]1CC[C@@H](N(CCCOC)C(=O)CC(C)C)CC1]

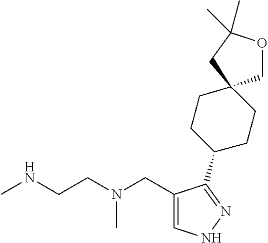 [SMILES: CNCCN(C)Cc1c[nH]nc1[C@H]1CC[C@]2(CC1)COC(C)(C)C2]

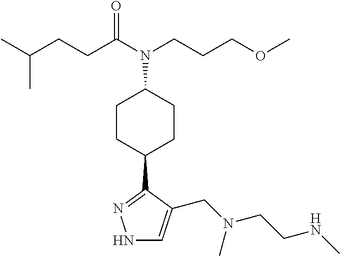 [SMILES: CNCCN(C)Cc1c[nH]nc1[C@H]1CC[C@H](N(CCCOC)C(=O)CCC(C)C)CC1]

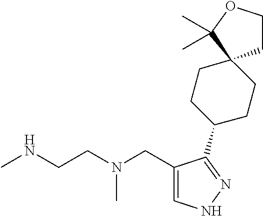 [SMILES: CNCCN(C)Cc1c[nH]nc1[C@H]1CC[C@@]2(CCOC2(C)C)CC1]

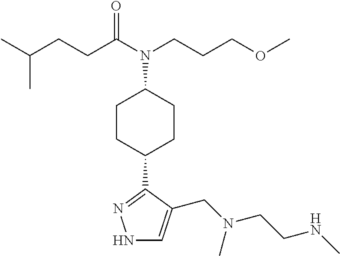 [SMILES: CNCCN(C)Cc1c[nH]nc1[C@H]1CC[C@@H](N(CCCOC)C(=O)CCC(C)C)CC1]

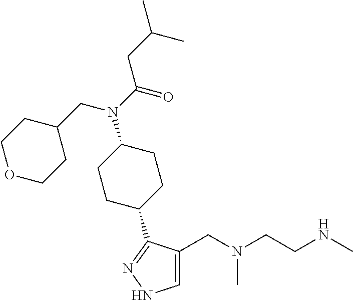 [SMILES: CNCCN(C)Cc1c[nH]nc1[C@H]1CC[C@@H](N(CC2CCOCC2)C(=O)CC(C)C)CC1]

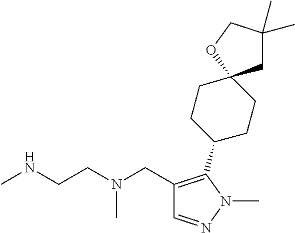

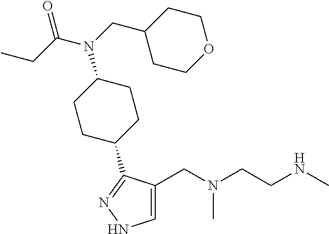 [SMILES: CCC(=O)N(CC1CCOCC1)[C@H]1CC[C@@H](c2n[nH]cc2CN(C)CCNC)CC1]

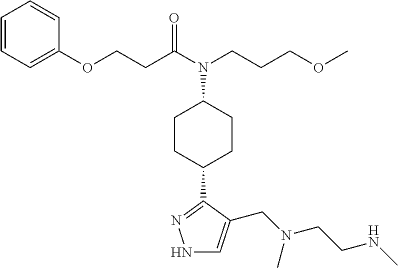 [SMILES: CNCCN(C)Cc1c[nH]nc1[C@H]1CC[C@@H](N(CCCOC)C(=O)CCOc2ccccc2)CC1]

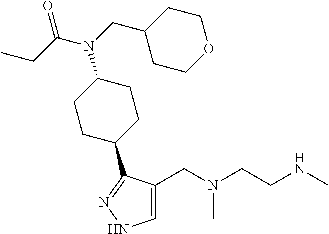 [SMILES: CCC(=O)N(CC1CCOCC1)[C@H]1CC[C@H](c2n[nH]cc2CN(C)CCNC)CC1]

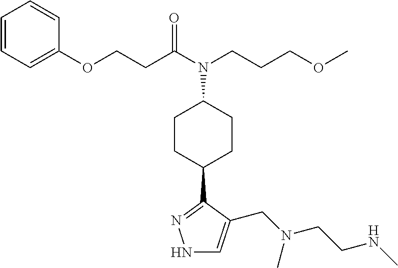 [SMILES: CNCCN(C)Cc1c[nH]nc1[C@H]1CC[C@H](N(CCCOC)C(=O)CCOc2ccccc2)CC1]

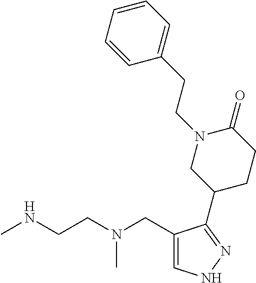 [SMILES: CNCCN(C)Cc1c[nH]nc1C1CCC(=O)N(CCc2ccccc2)C1]

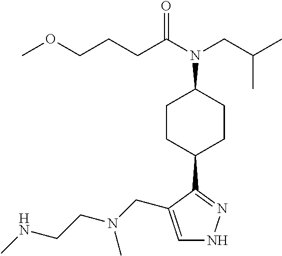 [SMILES: CNCCN(C)Cc1c[nH]nc1[C@H]1CC[C@@H](N(CC(C)C)C(=O)CCCOC)CC1]

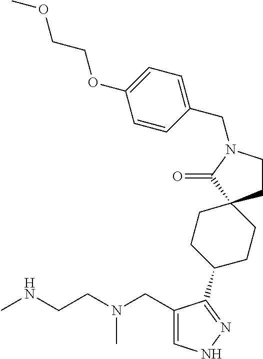 [SMILES: CNCCN(C)Cc1c[nH]nc1[C@H]1CC[C@]2(CCN(Cc3ccc(OCCOC)cc3)C2=O)CC1]

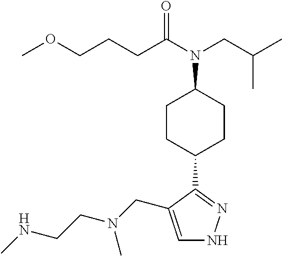 [SMILES: CNCCN(C)Cc1c[nH]nc1[C@H]1CC[C@H](N(CC(C)C)C(=O)CCCOC)CC1]

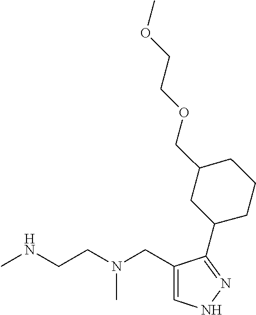 [SMILES: CNCCN(C)Cc1c[nH]nc1C1CCCC(COCCOC)C1]

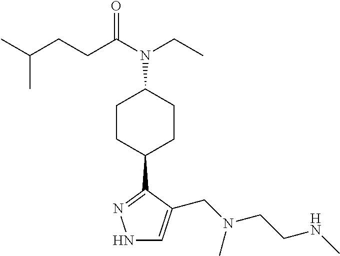 [SMILES: CCN(C(=O)CCC(C)C)[C@H]1CC[C@H](c2n[nH]cc2CN(C)CCNC)CC1]

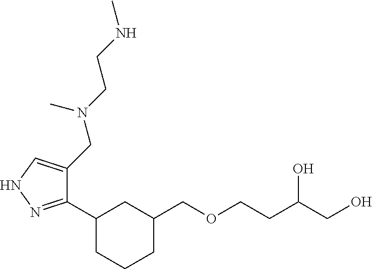 [SMILES: CNCCN(C)Cc1c[nH]nc1C1CCCC(COCCC(O)CO)C1]

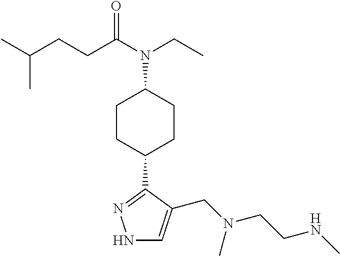 [SMILES: CCN(C(=O)CCC(C)C)[C@H]1CC[C@@H](c2n[nH]cc2CN(C)CCNC)CC1]

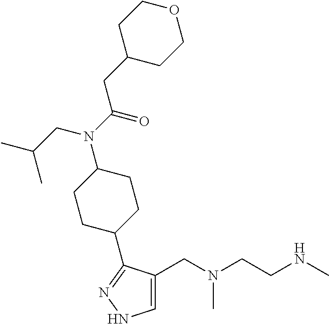 [SMILES: CNCCN(C)Cc1c[nH]nc1C1CCC(N(CC(C)C)C(=O)CC2CCOCC2)CC1]

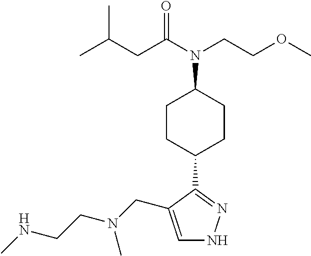

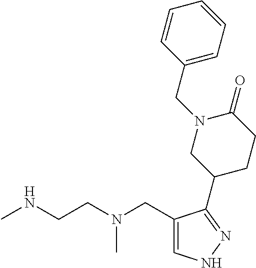 [SMILES: CNCCN(C)Cc1c[nH]nc1C1CCC(=O)N(Cc2ccccc2)C1]

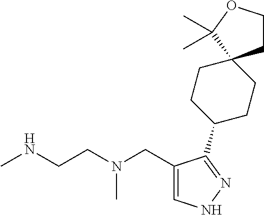 [SMILES: CNCCN(C)Cc1c[nH]nc1[C@H]1CC[C@]2(CCOC2(C)C)CC1]

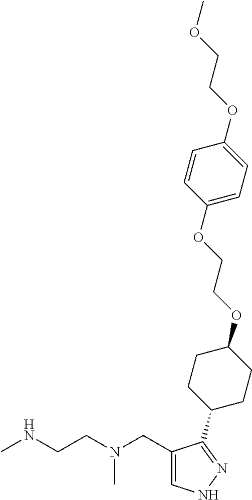 [SMILES: CNCCN(C)Cc1c[nH]nc1[C@H]1CC[C@H](OCCOc2ccc(OCCOC)cc2)CC1]

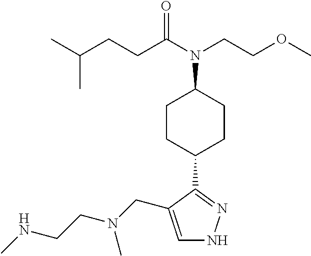

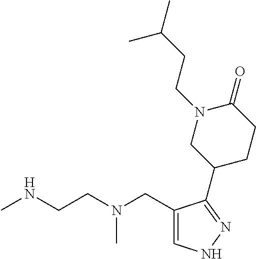 [SMILES: CNCCN(C)Cc1c[nH]nc1C1CCC(=O)N(CCC(C)C)C1]

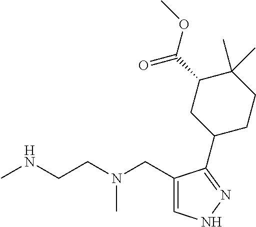

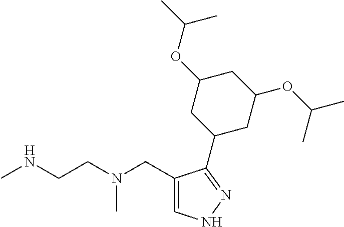 [SMILES: CNCCN(C)Cc1c[nH]nc1C1CC(OC(C)C)CC(OC(C)C)C1]

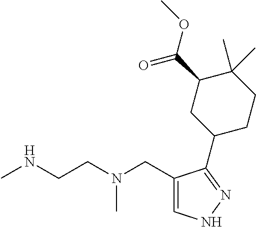

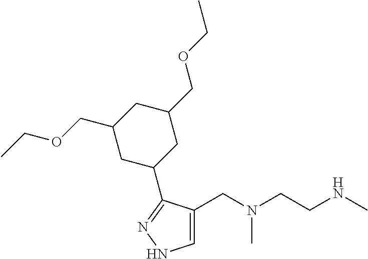 [SMILES: CCOCC1CC(COCC)CC(c2n[nH]cc2CN(C)CCNC)C1]

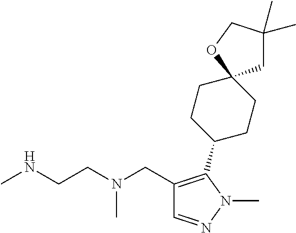

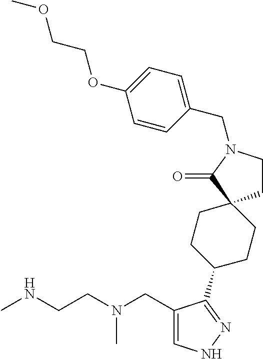 [SMILES: CNCCN(C)Cc1c[nH]nc1[C@H]1CC[C@@]2(CCN(Cc3ccc(OCCOC)cc3)C2=O)CC1]

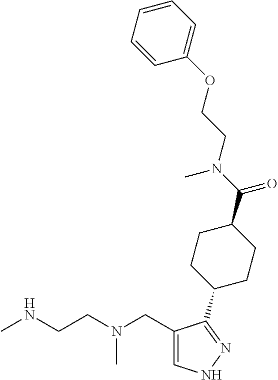 [SMILES: CNCCN(C)Cc1c[nH]nc1[C@H]1CC[C@H](C(=O)N(C)CCOc2ccccc2)CC1]

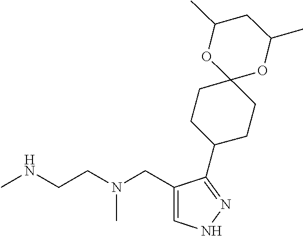

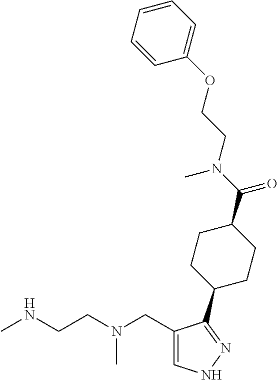 [SMILES: CNCCN(C)Cc1c[nH]nc1[C@H]1CC[C@@H](C(=O)N(C)CCOc2ccccc2)CC1]

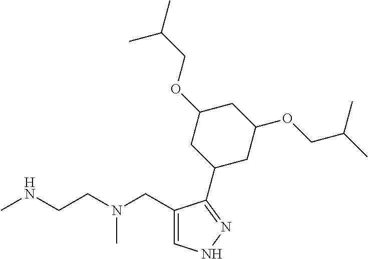 [SMILES: CNCCN(C)Cc1c[nH]nc1C1CC(OCC(C)C)CC(OCC(C)C)C1]

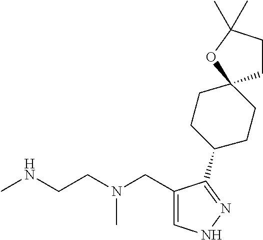 [SMILES: CNCCN(C)Cc1c[nH]nc1[C@H]1CC[C@@]2(CCC(C)(C)O2)CC1]

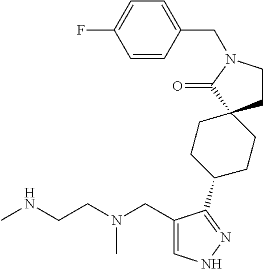 [SMILES: CNCCN(C)Cc1c[nH]nc1[C@H]1CC[C@]2(CCN(Cc3ccc(F)cc3)C2=O)CC1]

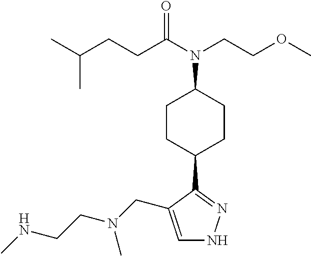

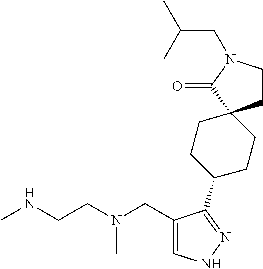 [SMILES: CNCCN(C)Cc1c[nH]nc1[C@H]1CC[C@]2(CCN(CC(C)C)C2=O)CC1]

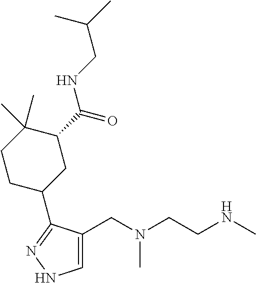 [SMILES: CNCCN(C)Cc1c[nH]nc1C1CCC(C)(C)[C@H](C(=O)NCC(C)C)C1]

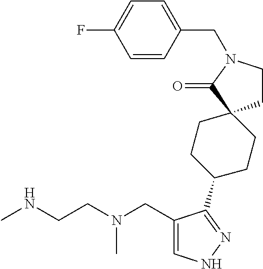 [SMILES: CNCCN(C)Cc1c[nH]nc1[C@H]1CC[C@@]2(CCN(Cc3ccc(F)cc3)C2=O)CC1]

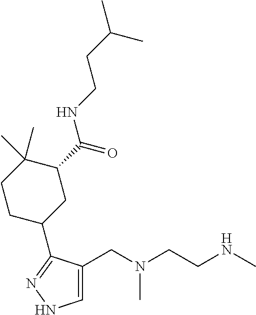 [SMILES: CNCCN(C)Cc1c[nH]nc1C1CCC(C)(C)[C@H](C(=O)NCCC(C)C)C1]

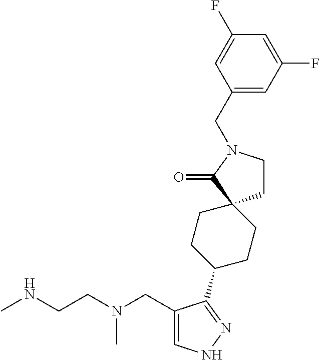 [SMILES: CNCCN(C)Cc1c[nH]nc1[C@H]1CC[C@@]2(CCN(Cc3cc(F)cc(F)c3)C2=O)CC1]

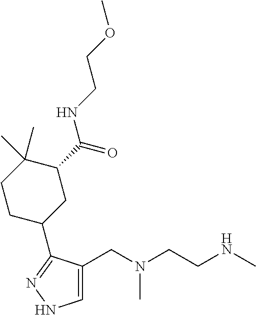 [SMILES: CNCCN(C)Cc1c[nH]nc1C1CCC(C)(C)[C@H](C(=O)NCCOC)C1]

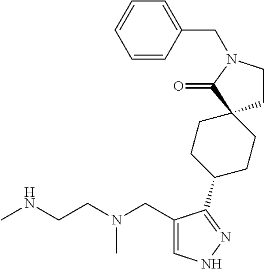 [SMILES: CNCCN(C)Cc1c[nH]nc1[C@H]1CC[C@@]2(CCN(Cc3ccccc3)C2=O)CC1]

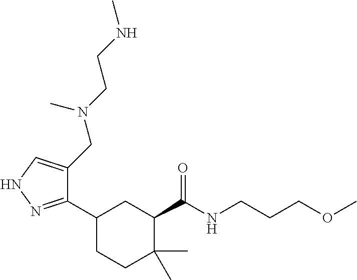 [SMILES: CNCCN(C)Cc1c[nH]nc1C1CCC(C)(C)[C@H](C(=O)NCCCOC)C1]

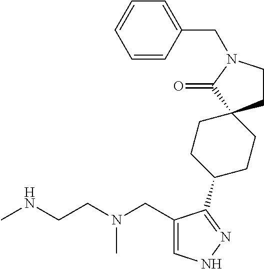 [SMILES: CNCCN(C)Cc1c[nH]nc1[C@H]1CC[C@]2(CCN(Cc3ccccc3)C2=O)CC1]

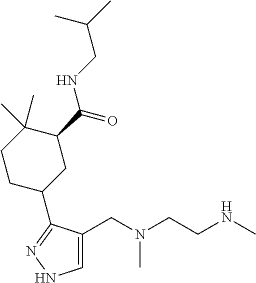 [SMILES: CNCCN(C)Cc1c[nH]nc1C1CCC(C)(C)[C@@H](C(=O)NCC(C)C)C1]

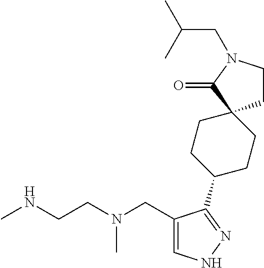 [SMILES: CNCCN(C)Cc1c[nH]nc1[C@H]1CC[C@@]2(CCN(CC(C)C)C2=O)CC1]

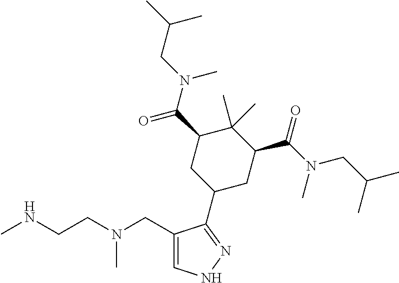 [SMILES: CNCCN(C)Cc1c[nH]nc1C1C[C@H](C(=O)N(C)CC(C)C)C(C)(C)[C@H](C(=O)N(C)CC(C)C)C1]

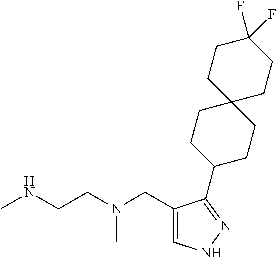 [SMILES: CNCCN(C)Cc1c[nH]nc1C1CCC2(CC1)CCC(F)(F)CC2]

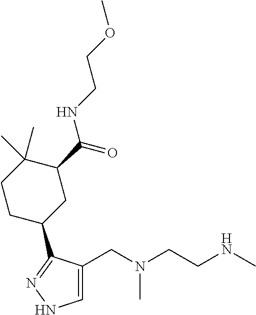 [SMILES: CNCCN(C)Cc1c[nH]nc1[C@H]1CCC(C)(C)[C@@H](C(=O)NCCOC)C1]

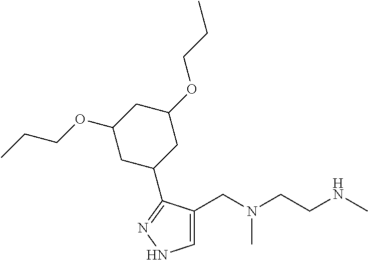 [SMILES: CCCOC1CC(OCCC)CC(c2n[nH]cc2CN(C)CCNC)C1]

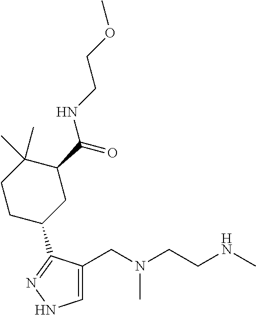 [SMILES: CNCCN(C)Cc1c[nH]nc1[C@@H]1CCC(C)(C)[C@@H](C(=O)NCCOC)C1]

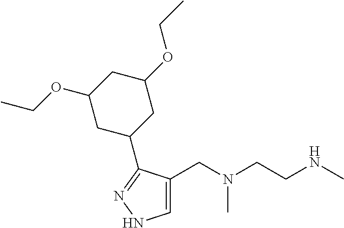 [SMILES: CCOC1CC(OCC)CC(c2n[nH]cc2CN(C)CCNC)C1]

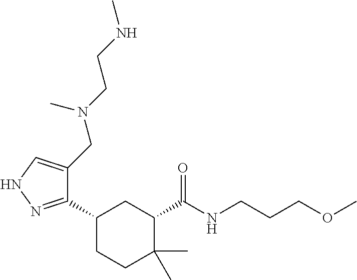 [SMILES: CNCCN(C)Cc1c[nH]nc1[C@H]1CCC(C)(C)[C@@H](C(=O)NCCCOC)C1]

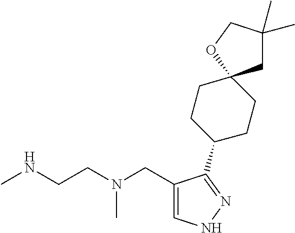

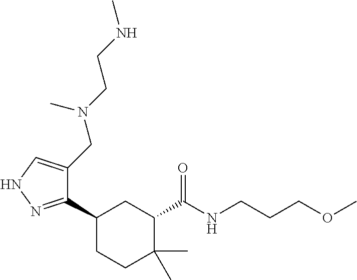 [SMILES: CNCCN(C)Cc1c[nH]nc1[C@@H]1CCC(C)(C)[C@@H](C(=O)NCCCOC)C1]

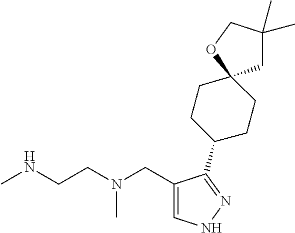

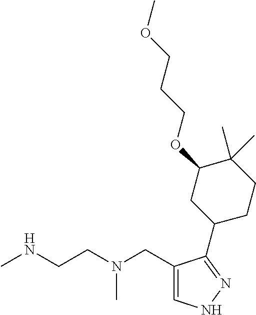 [SMILES: CNCCN(C)Cc1c[nH]nc1C1CCC(C)(C)[C@H](OCCCOC)C1]

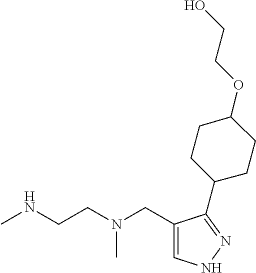 [SMILES: CNCCN(C)Cc1c[nH]nc1C1CCC(OCCO)CC1]

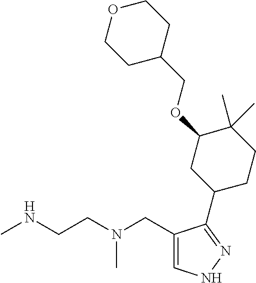 [SMILES: CNCCN(C)Cc1c[nH]nc1C1CCC(C)(C)[C@H](OCC2CCOCC2)C1]

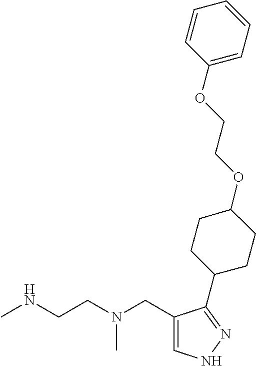 [SMILES: CNCCN(C)Cc1c[nH]nc1C1CCC(OCCOc2ccccc2)CC1]

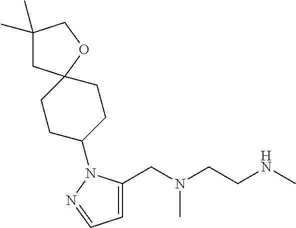

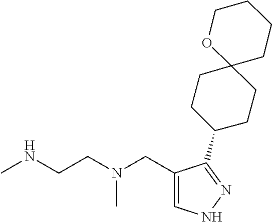

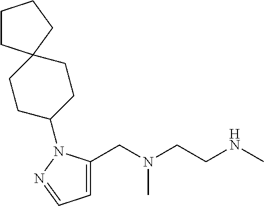 [SMILES: CNCCN(C)Cc1ccnn1C1CCC2(CCCC2)CC1]

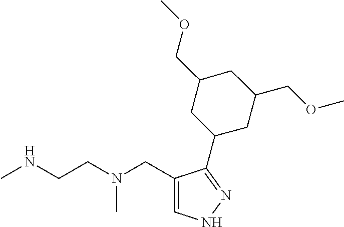

In certain embodiments, a provided compound is a compound selected from any one of the compounds provided in Table 2, or a pharmaceutically acceptable salt thereof.

TABLE 2
Exemplified Compounds and Biological Activities

| | | | | | PRMT1 |
|---|---|---|---|---|---|
| | | | | | RGG |
| Cpd. | | PRMT1 | PRMT6 | PRMT8 | ICW EC₃₀ |
| No. | Compound Structure | IC₅₀ (μM) | IC₅₀ (μM) | IC₅₀ (μM) | (μM) |
| 1. | 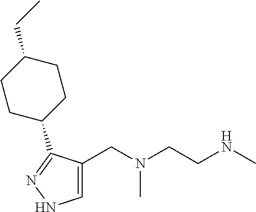 [SMILES: CC[C@H]1CC[C@@H](c2n[nH]cc2CN(C)CCNC)CC1] | 0.0048 | 0.01021 | 0.01115 | 0.08301 |
| 2. | 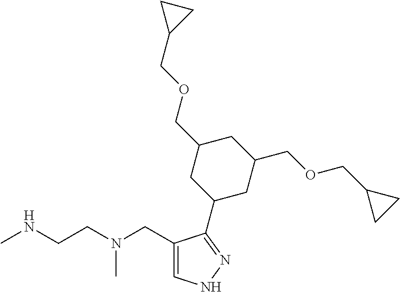 [SMILES: CNCCN(C)Cc1c[nH]nc1C1CC(COCC2CC2)CC(COCC2CC2)C1] | 0.06925 | 0.74088 | 0.36532 | >5.0 |
| 3. | 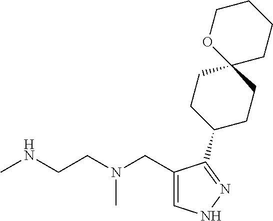 [SMILES: CNCCN(C)Cc1c[nH]nc1[C@H]1CC[C@@]2(CCCCO2)CC1] | 0.01357 | 0.0277 | 0.03275 | 0.11466 |
| 4. | 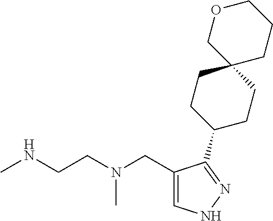 [SMILES: CNCCN(C)Cc1c[nH]nc1[C@H]1CC[C@]2(CCCOC2)CC1] | 0.00943 | 0.03312 | 0.01605 | 0.01834 |
| 5. | 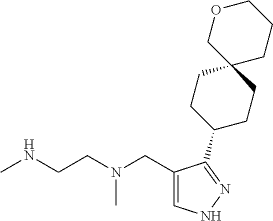 [SMILES: CNCCN(C)Cc1c[nH]nc1[C@H]1CC[C@@]2(CCCOC2)CC1] | 0.01165 | 0.0359 | 0.01771 | 0.01465 |
| 6. | 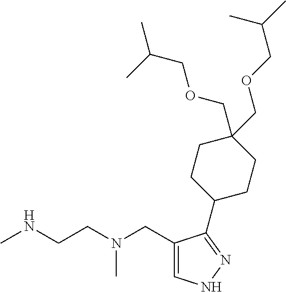 [SMILES: CNCCN(C)Cc1c[nH]nc1C1CCC(COCC(C)C)(COCC(C)C)CC1] | 0.01669 | 0.03196 | 0.06242 | 0.9493 |
| 9. | 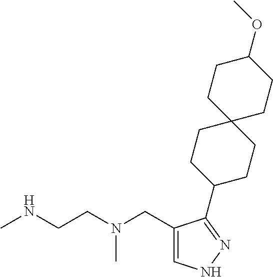 [SMILES: CNCCN(C)Cc1c[nH]nc1C1CCC2(CCC(OC)CC2)CC1] | 0.0087 | 0.01345 | 0.01111 | 0.01245 |
| 10. | 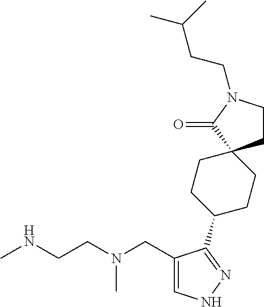 [SMILES: CNCCN(C)Cc1c[nH]nc1[C@H]1CC[C@]2(CCN(CCC(C)C)C2=O)CC1] | 0.05498 | 0.0385 | 0.10563 | 2.00085 |
| 11. | 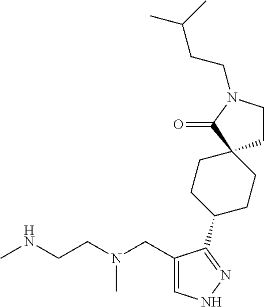 [SMILES: CNCCN(C)Cc1c[nH]nc1[C@H]1CC[C@@]2(CCN(CCC(C)C)C2=O)CC1] | 0.00655 | 0.01858 | 0.01024 | 0.03417 |
| 12. | 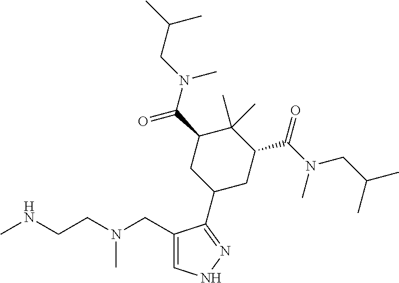 [SMILES: CNCCN(C)Cc1c[nH]nc1C1C[C@@H](C(=O)N(C)CC(C)C)C(C)(C)[C@H](C(=O)N(C)CC(C)C)C1] | >10.0 | >10.0 | >10.0 | >5.0 |
| 13. | 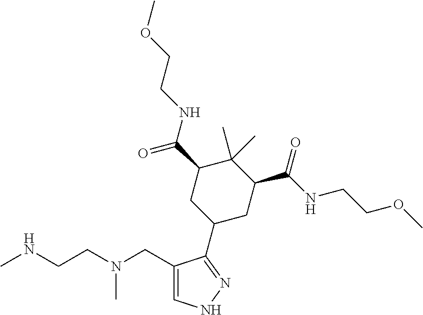 | >10.0 | >10.0 | >10.0 | >5.0 |
| 14. | 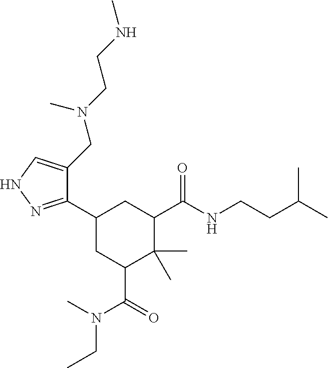 [SMILES: CCN(C)C(=O)C1CC(c2n[nH]cc2CN(C)CCNC)CC(C(=O)NCCC(C)C)C1(C)C] | >10.0 | >10.0 | >10.0 | >5.0 |
| 15. | 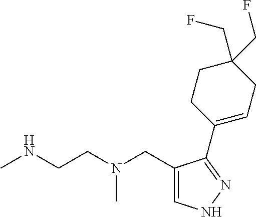 [SMILES: CNCCN(C)Cc1c[nH]nc1C1=CCC(CF)(CF)CC1] | 0.0056 | 0.01671 | 0.0153 | 0.36108 |
| 16. | 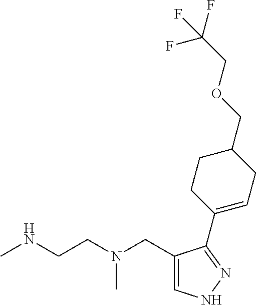 [SMILES: CNCCN(C)Cc1c[nH]nc1C1=CCC(COCC(F)(F)F)CC1] | 0.01119 | 0.01699 | 0.0264 | 0.83927 |
| 18. | 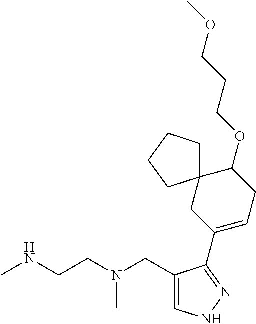 [SMILES: CNCCN(C)Cc1c[nH]nc1C1=CCC(OCCCOC)C2(CCCC2)C1] | 0.01368 | 0.02964 | 0.04247 | 0.6885 |
| 19. | 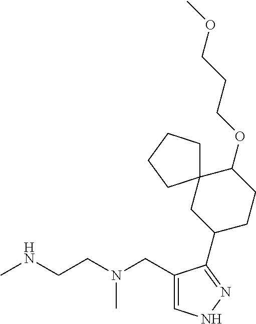 [SMILES: CNCCN(C)Cc1c[nH]nc1C1CCC(OCCCOC)C2(CCCC2)C1] | 0.01113 | 0.19145 | 0.03013 | 0.44029 |
| 20. | 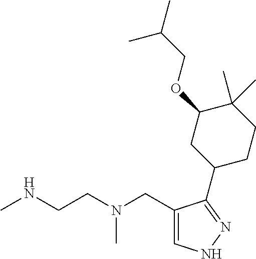 | 0.011 | 0.07475 | 0.02454 | 0.20429 |
| 21. | 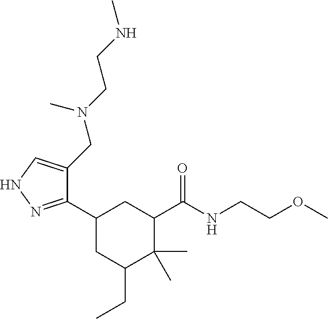 [SMILES: CCC1CC(c2n[nH]cc2CN(C)CCNC)CC(C(=O)NCCOC)C1(C)C] | 0.24564 | 0.78932 | 0.61386 | >5.0 |
| 22. | 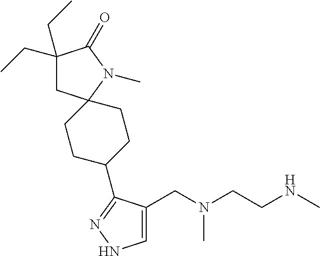 [SMILES: CCC1(CC)CC2(CCC(c3n[nH]cc3CN(C)CCNC)CC2)N(C)C1=O] | 0.01311 | 0.06269 | 0.02033 | 1.83515 |
| 23. | 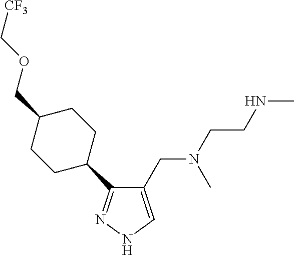 [SMILES: CNCCN(C)Cc1c[nH]nc1[C@H]1CC[C@@H](COCC(F)(F)F)CC1] | 0.05083 | 0.0785 | 0.09179 | 1.72102 |
| 24. | 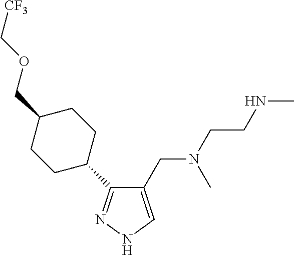 [SMILES: CNCCN(C)Cc1c[nH]nc1[C@H]1CC[C@H](COCC(F)(F)F)CC1] | 0.01239 | 0.02156 | 0.01833 | 0.16163 |
| 25. | 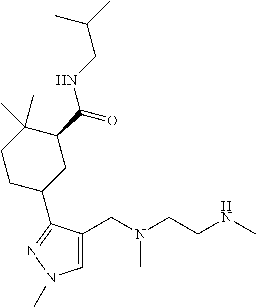 [SMILES: CNCCN(C)Cc1cn(C)nc1C1CCC(C)(C)[C@@H](C(=O)NCC(C)C)C1] | 0.70192 | 1.61385 | 2.78558 | >5.0 |
| 26. | 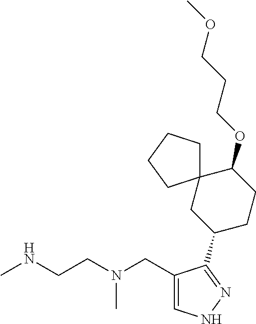 [SMILES: CNCCN(C)Cc1c[nH]nc1[C@H]1CC[C@H](OCCCOC)C2(CCCC2)C1] | 0.0143 | 0.10698 | 0.06756 | 0.21719 |
| 27. | 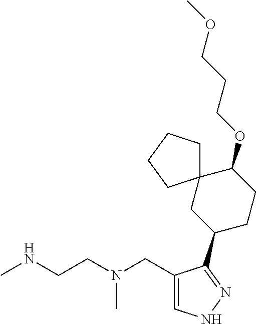 [SMILES: CNCCN(C)Cc1c[nH]nc1[C@@H]1CC[C@H](OCCCOC)C2(CCCC2)C1] | 0.00658 | 0.38266 | 0.03116 | 0.21637 |
| 28. | 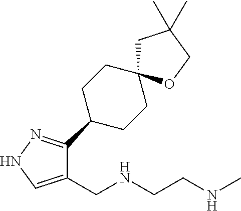 [SMILES: CNCCNCc1c[nH]nc1[C@H]1CC[C@@]2(CC1)CC(C)(C)CO2] | 0.01317 | 0.26196 | 0.02073 | — |
| 29. | 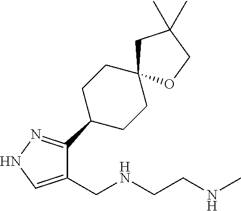 [SMILES: CNCCNCc1c[nH]nc1[C@H]1CC[C@]2(CC1)CC(C)(C)CO2] | 0.19336 | 1.90803 | 0.25835 | — |
| 30. | 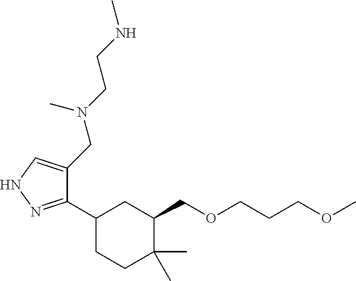 [SMILES: CNCCN(C)Cc1c[nH]nc1C1CCC(C)(C)[C@H](COCCCOC)C1] | 0.02096 | 0.06029 | 0.187 | 0.209 |
| 31. | 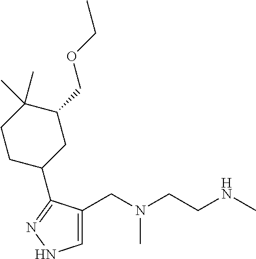 [SMILES: CCOC[C@@H]1CC(c2n[nH]cc2CN(C)CCNC)CCC1(C)C] | 0.02497 | 0.04559 | 0.32485 | 0.45365 |
| 32. | 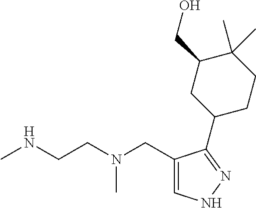 | 0.02212 | 0.01629 | 0.29132 | 0.51789 |
| 33. | 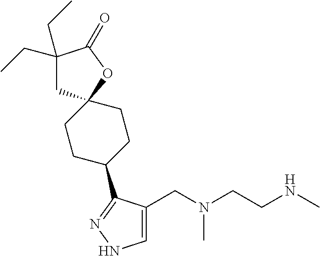 [SMILES: CCC1(CC)C[C@]2(CC[C@H](c3n[nH]cc3CN(C)CCNC)CC2)OC1=O] | 0.01323 | 0.0208 | 0.09579 | 0.04081 |
| 34. | 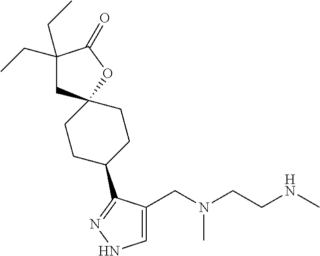 [SMILES: CCC1(CC)C[C@]2(CC[C@@H](c3n[nH]cc3CN(C)CCNC)CC2)OC1=O] | 0.01518 | 0.01061 | 0.14635 | 0.06546 |
| 35. | 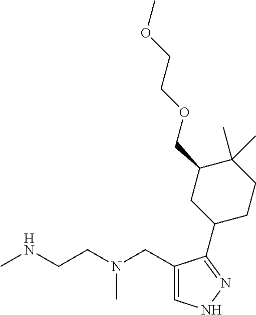 | 0.05191 | 0.12411 | 0.11036 | 0.3301 |
| 36. | 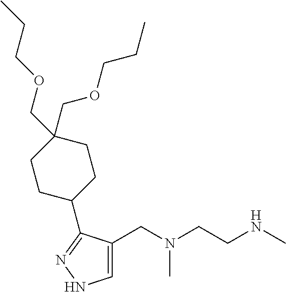 [SMILES: CCCOCC1(COCCC)CCC(c2n[nH]cc2CN(C)CCNC)CC1] | 0.00793 | 0.01493 | 0.0124 | 0.06617 |
| 37. | 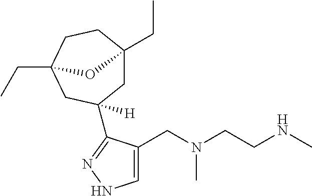 [SMILES: CC[C@]12CC[C@](CC)(C[C@H](c3n[nH]cc3CN(C)CCNC)C1)O2] | 0.02517 | 0.1278 | 0.04547 | 0.24548 |
| 38. | 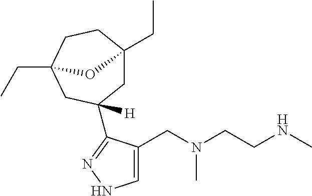 [SMILES: CC[C@]12CC[C@](CC)(C[C@@H](c3n[nH]cc3CN(C)CCNC)C1)O2] | 0.11128 | 0.13199 | 0.17654 | >5.0 |
| 39. | 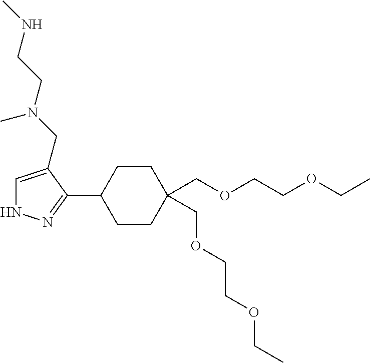 [SMILES: CCOCCOCC1(COCCOCC)CCC(c2n[nH]cc2CN(C)CCNC)CC1] | 0.03119 | 0.04197 | 0.13857 | 1.38791 |
| 40. | 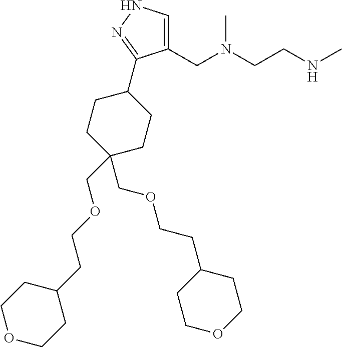 [SMILES: CNCCN(C)Cc1c[nH]nc1C1CCC(COCCC2CCOCC2)(COCCC2CCOCC2)CC1] | 0.02072 | 0.0374 | 0.04551 | 0.45848 |
| 41. | 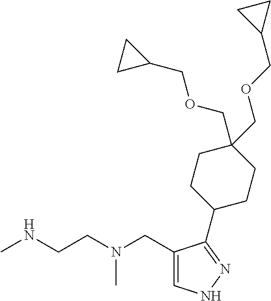 [SMILES: CNCCN(C)Cc1c[nH]nc1C1CCC(COCC2CC2)(COCC2CC2)CC1] | 0.00433 | 0.00694 | 0.00927 | 0.06748 |
| 42. | 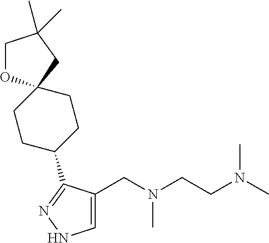 [SMILES: CN(C)CCN(C)Cc1c[nH]nc1[C@H]1CC[C@@]2(CC1)CC(C)(C)CO2] | 0.03148 | 0.09085 | 0.03377 | 0.05141 |
| 43. | 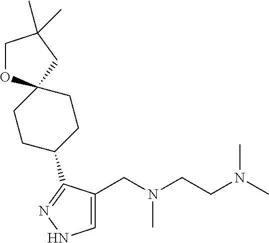 [SMILES: CN(C)CCN(C)Cc1c[nH]nc1[C@H]1CC[C@]2(CC1)CC(C)(C)CO2] | 0.05703 | 0.2879 | 0.09717 | 0.62278 |
| 44. | 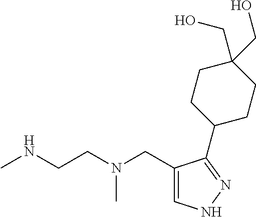 [SMILES: CNCCN(C)Cc1c[nH]nc1C1CCC(CO)(CO)CC1] | 0.0213 | 0.02154 | 0.0344 | >5.0 |
| 45. | 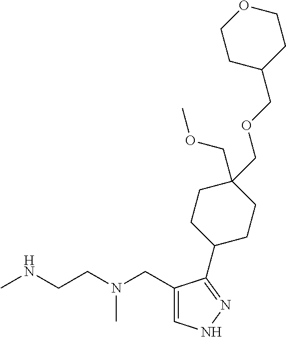 [SMILES: CNCCN(C)Cc1c[nH]nc1C1CCC(COC)(COCC2CCOCC2)CC1] | 0.03302 | 0.05375 | 0.01116 | 0.10945 |
| 46. | 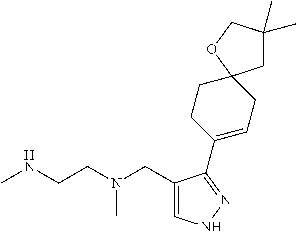 | 0.01982 | 0.05967 | 0.02462 | 0.18506 |
| 47. | 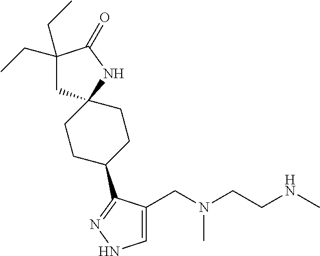 [SMILES: CCC1(CC)C[C@]2(CC[C@H](c3n[nH]cc3CN(C)CCNC)CC2)NC1=O] | 0.01524 | 0.01577 | 0.01488 | 0.36706 |
| 48. | 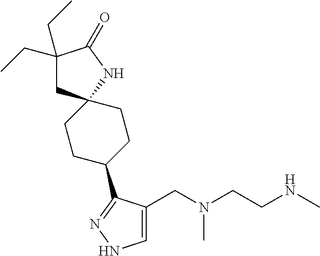 [SMILES: CCC1(CC)C[C@]2(CC[C@@H](c3n[nH]cc3CN(C)CCNC)CC2)NC1=O] | 0.03526 | 0.07355 | 0.07058 | >5.0 μM |
| 49. | 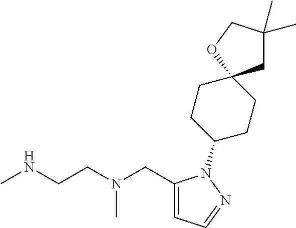 | 0.01928 | 0.12803 | 0.01669 | 0.11126 |
| 50. | 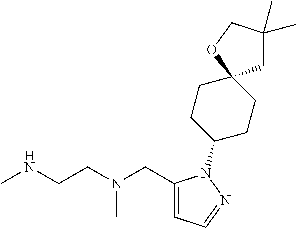 | 0.02091 | 0.5021 | 0.03224 | 0.32124 |
| 51. | 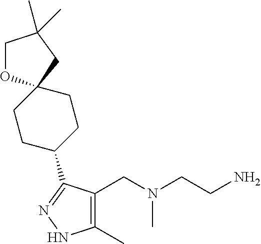 [SMILES: Cc1[nH]nc([C@H]2CC[C@@]3(CC2)CC(C)(C)CO3)c1CN(C)CCN] | 0.01196 | 0.15509 | 0.01425 | 0.16858 |
| 52. | 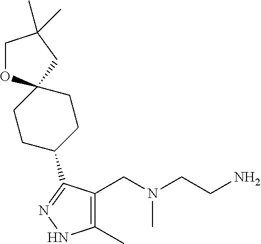 [SMILES: Cc1[nH]nc([C@H]2CC[C@]3(CC2)CC(C)(C)CO3)c1CN(C)CCN] | 0.01598 | 0.20175 | 0.02313 | 0.37358 |
| 53. | 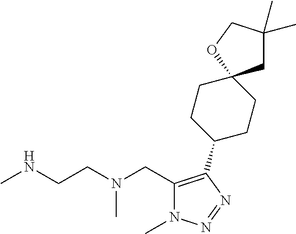 | 5.85358 | >10.0 | — | — |
| 54. | 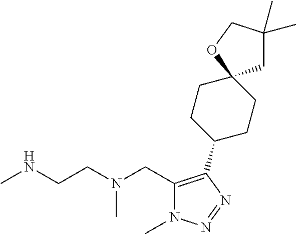 | 9.30864 | 9.92045 | — | — |
| 55. | 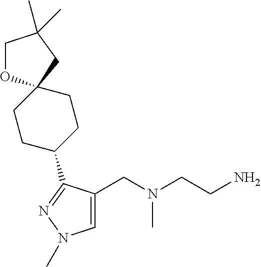 [SMILES: CN(CCN)Cc1cn(C)nc1[C@H]1CC[C@@]2(CC1)CC(C)(C)CO2] | 0.00728 | 0.19051 | — | — |
| 56. | 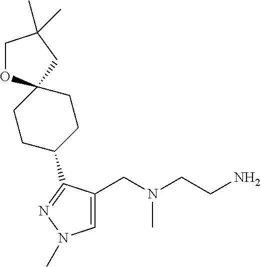 [SMILES: CN(CCN)Cc1cn(C)nc1[C@H]1CC[C@]2(CC1)CC(C)(C)CO2] | 0.03101 | 0.85006 | — | — |
| 57. | 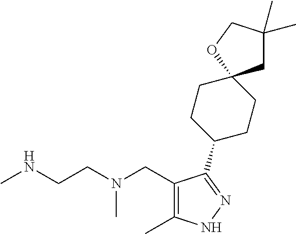 | 0.0258 | 0.09404 | 0.03274 | 0.04032 |
| 58. | 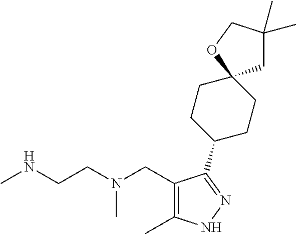 | 0.03868 | 0.16259 | 0.05507 | 0.08334 |
| 59. | 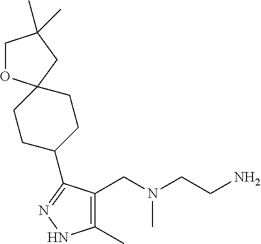 [SMILES: Cc1[nH]nc(C2CCC3(CC2)CC(C)(C)CO3)c1CN(C)CCN] | 0.01138 | 0.13051 | 0.01751 | 0.19307 |
| 60. | 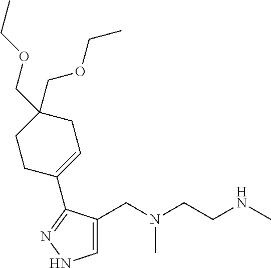 [SMILES: CCOCC1(COCC)CC=C(c2n[nH]cc2CN(C)CCNC)CC1] | 0.01487 | 0.04108 | 0.02246 | 0.04467 |
| 61. | 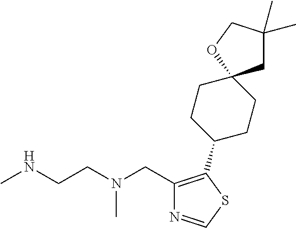 | 0.00848 | 0.0284 | 0.01141 | 0.01849 |
| 62. | 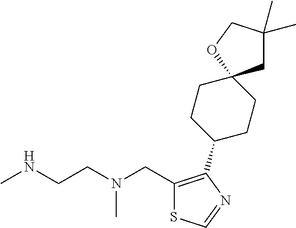 | 0.00418 | 0.01828 | 0.00908 | 0.00696 |
| 63. | 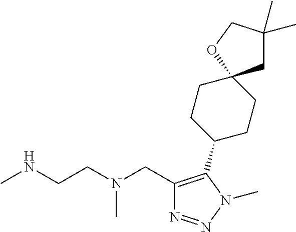 | — | — | — | — |
| 64. | 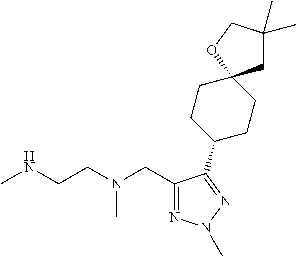 | 0.03193 | 0.20779 | 0.02534 | 0.11898 |
| 65. | 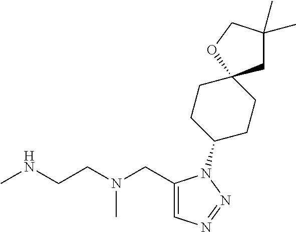 | >10.0 | >10.0 | >10.0 | — |
| 66. | 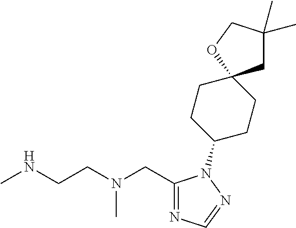 | — | — | — | — |
| 67. | 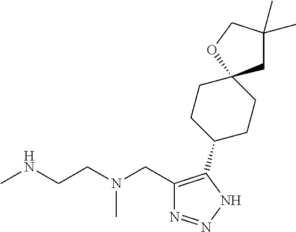 | 0.01149 | 0.05114 | 0.01978 | 0.0339 |
| 68. | 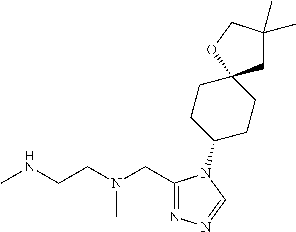 | 4.33168 | >10.0 | 3.47812 | — |
| 69. | 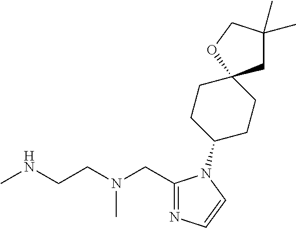 | 0.25508 | 8.04125 | 0.27294 | — |
| 70. | 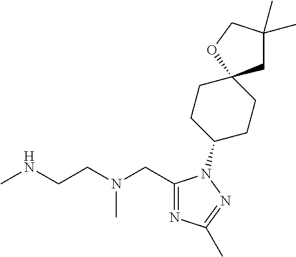 | — | — | — | — |
| 71. | 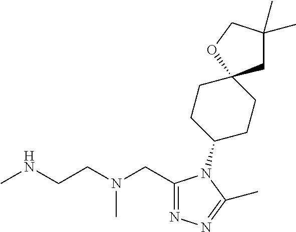 | 5.10199 | >10.0 | 4.32027 | — |
| 72. | 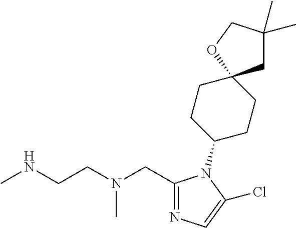 | — | — | — | — |
| 73. | 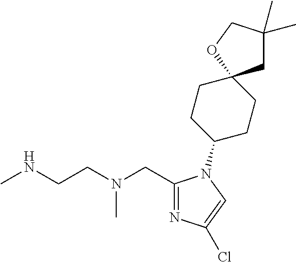 | — | — | — | — |
| 74. | 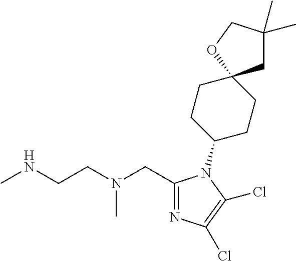 | — | — | — | — |
| 75. | 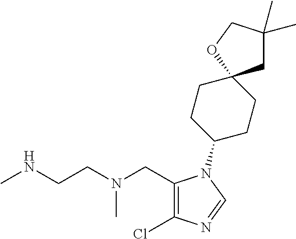 | — | — | — | — |
| 76. | 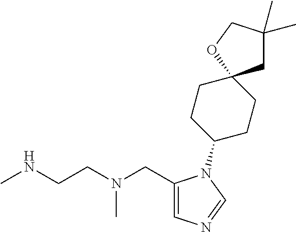 | 0.01462 | 0.18081 | 0.01569 | 0.38941 |
| 77. | 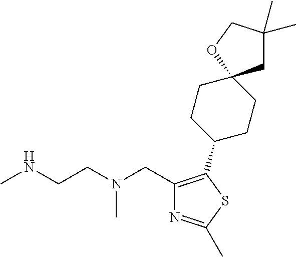 | 0.02291 | 0.18008 | 0.02772 | 0.05311 |
| 78. | 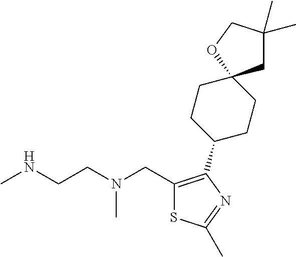 | 0.02298 | 0.0984 | 0.03376 | 0.02959 |
| 79. | 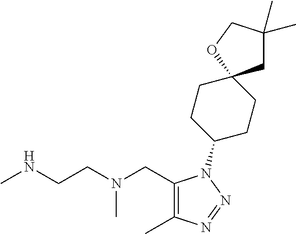 | — | — | — | — |
| 80. | 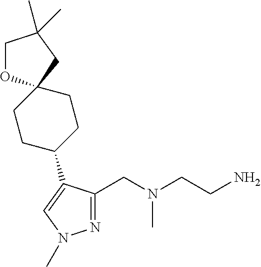 [SMILES: CN(CCN)Cc1nn(C)cc1[C@H]1CC[C@@]2(CC1)CC(C)(C)CO2] | 0.00583 | 0.34466 | 0.01209 | 0.06159 |
| 81. | 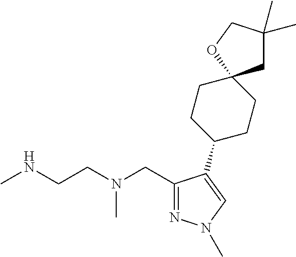 | 0.02279 | 0.12309 | 0.03087 | 0.09594 |
| 83. | 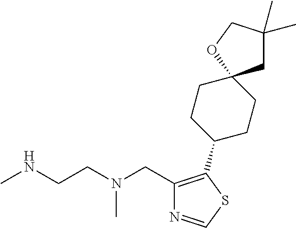 | 0.01481 | 0.06269 | 0.01853 | 0.05796 |
| 84. | 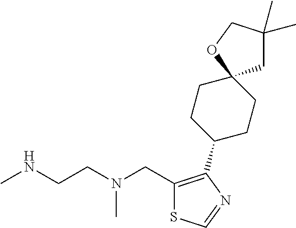 | 0.01402 | 0.08934 | 0.02422 | 0.05469 |
| 85. | 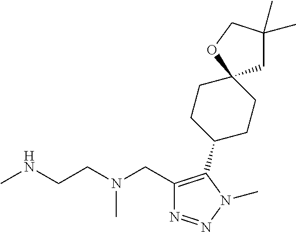 | — | — | — | — |
| 86. | 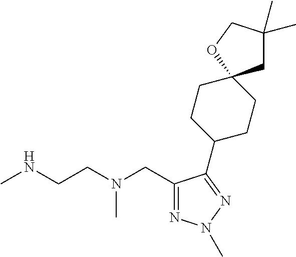 | 0.05006 | 0.4443 | 0.04986 | 0.44364 |
| 87. | 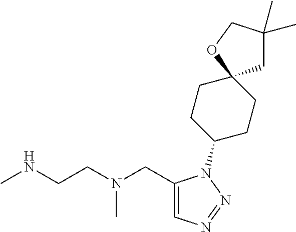 | — | — | — | — |
| 88. | 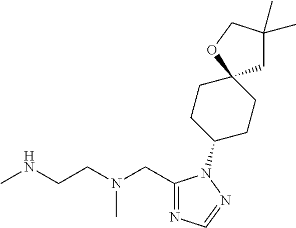 | — | — | — | — |
| 89. | 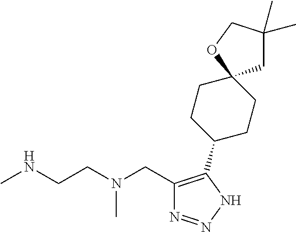 | 0.05969 | 0.42762 | 0.13724 | 0.76044 |
| 90. | 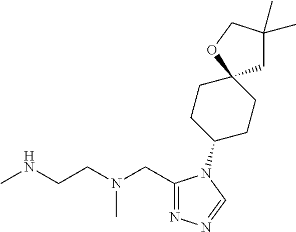 | >10.0 | >10.0 | >10.0 | — |
| 91. | 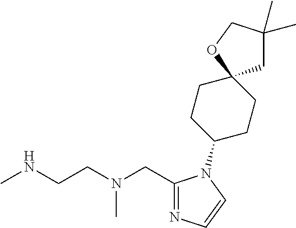 | 1.0209 | >10.0 | 1.18423 | — |
| 92. | 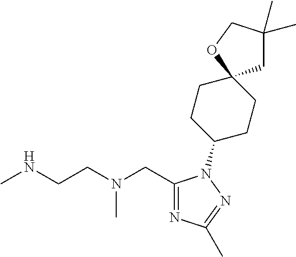 | — | — | — | — |
| 93. | 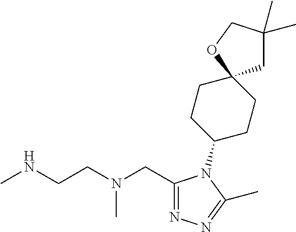 | >10.0 | >10.0 | >10.0 | — |
| 94. | 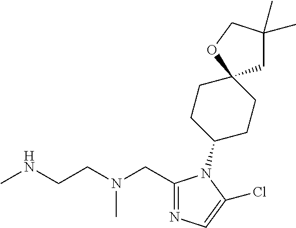 | — | — | — | — |
| 95. | 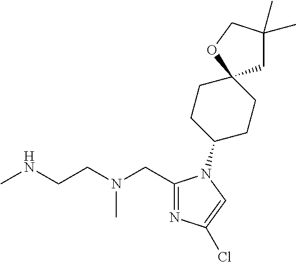 | — | — | — | — |
| 96. | 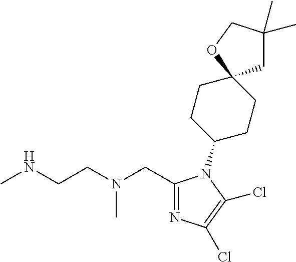 | — | — | — | — |
| 97. | 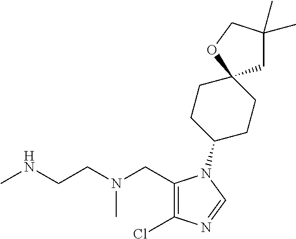 | 0.31816 | 3.51408 | 0.52597 | >10 |
| 98. | 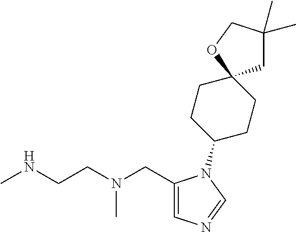 | 4.36049 | 2.78857 | 3.68054 | — |
| 99. | 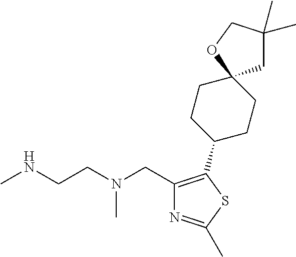 | 0.03564 | 0.317 | 0.04902 | 0.09553 |
| 100. | 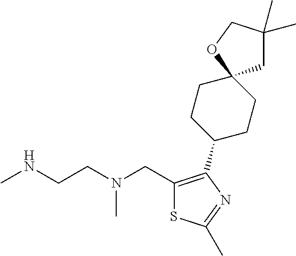 | 0.03352 | 0.38932 | 0.04356 | 0.07586 |
| 101. | 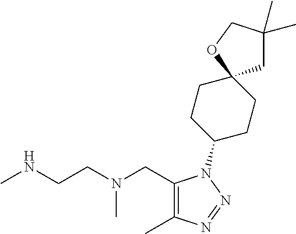 | — | — | — | — |
| 102. | 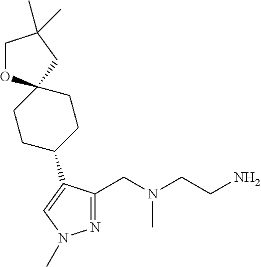 [SMILES: CN(CCN)Cc1nn(C)cc1[C@H]1CC[C@]2(CC1)CC(C)(C)CO2] | 0.0212 | 1.13868 | 0.0389 | 0.44674 |
| 103. | 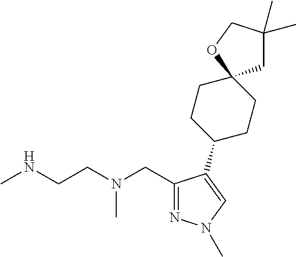 | 0.03717 | 0.40511 | 0.04271 | 0.59738 |
| 105. | 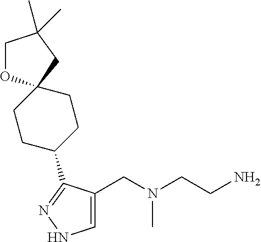 [SMILES: CN(CCN)Cc1c[nH]nc1[C@H]1CC[C@@]2(CC1)CC(C)(C)CO2] | 0.00568 | 0.01846 | 0.00913 | 0.01212 |
| 106. | 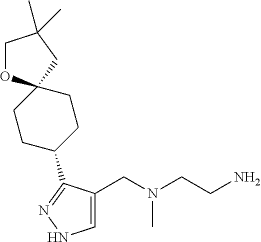 [SMILES: CN(CCN)Cc1c[nH]nc1[C@H]1CC[C@]2(CC1)CC(C)(C)CO2] | 0.04527 | 0.01276 | 0.06719 | 0.04527 |
| 107. | 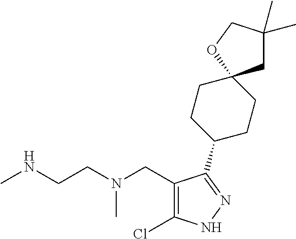 | — | — | — | — |
| 108. | 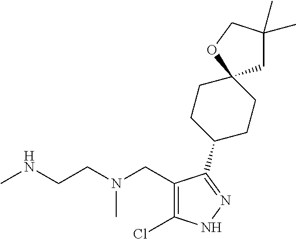 | — | — | — | — |
| 109. | 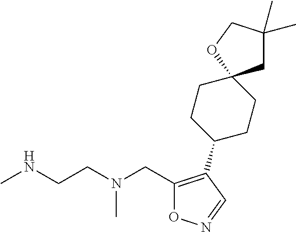 | — | — | — | — |
| 110. | 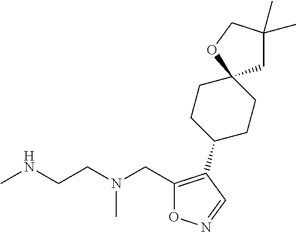 | — | — | — | — |
| 111. | 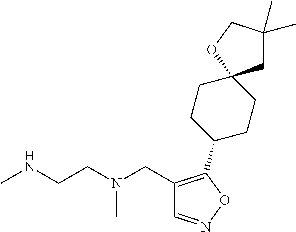 | — | — | — | — |
| 112. | 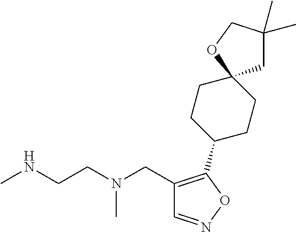 | — | — | — | — |
| 113. | 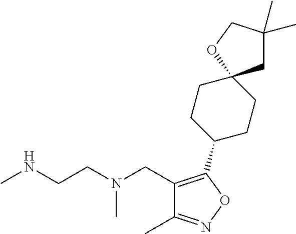 | 0.07954 | 1.15453 | 0.05146 | — |
| 114. | 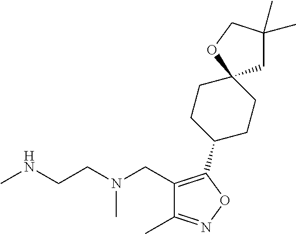 | 0.12118 | 2.08042 | 0.06795 | >10 |
| 115. | 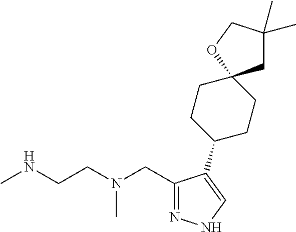 | 0.01152 | 0.04163 | 0.01623 | 0.02034 |
| 116. | 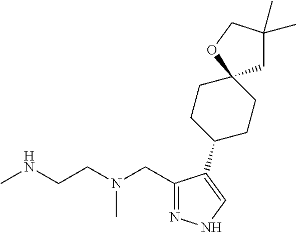 | 0.02286 | 0.13436 | 0.03183 | 0.15455 |
| 117. | 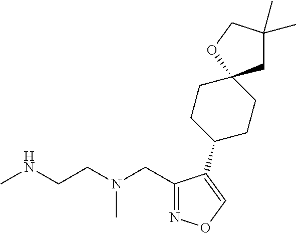 | 0.05127 | 1.30968 | 0.04082 | 0.86323 |
| 118. | 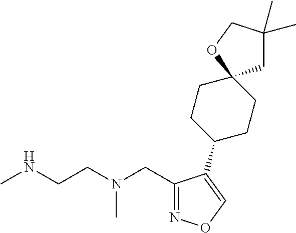 | 0.40882 | 3.53323 | 0.48206 | — |
| 119. | 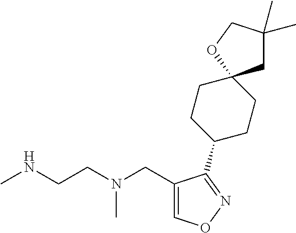 | — | — | — | — |
| 120. | 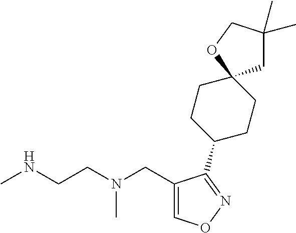 | — | — | — | — |
| 121. | 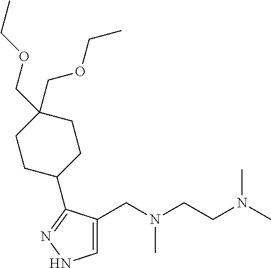 [SMILES: CCOCC1(COCC)CCC(c2n[nH]cc2CN(C)CCN(C)C)CC1] | 0.03799 | 0.07929 | 0.05818 | 0.11748 |
| 122. | 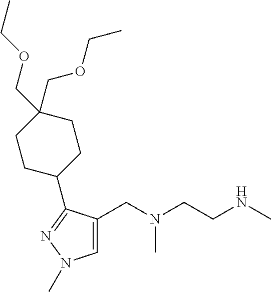 [SMILES: CCOCC1(COCC)CCC(c2nn(C)cc2CN(C)CCNC)CC1] | 0.02114 | 0.04275 | 0.0283 | 0.02211 |
| 123. | 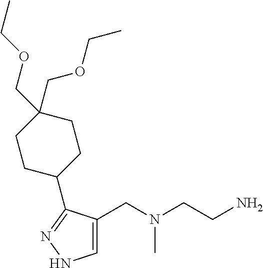 [SMILES: CCOCC1(COCC)CCC(c2n[nH]cc2CN(C)CCN)CC1] | 0.00428 | 0.01674 | 0.00949 | 0.0178 |
| 124. | 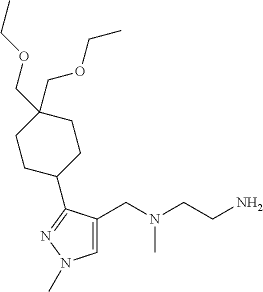 [SMILES: CCOCC1(COCC)CCC(c2nn(C)cc2CN(C)CCN)CC1] | 0.01367 | 0.05805 | 0.02545 | 0.05587 |
| 125. | 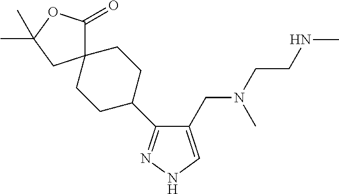 | 0.01066 | 0.01737 | 0.01574 | 0.01488 |
| 126. | 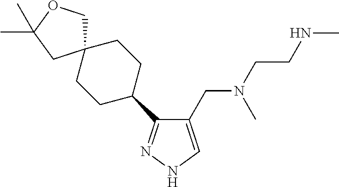 | 0.01429 | 0.03992 | 0.02129 | 0.01526 |
| 127. | 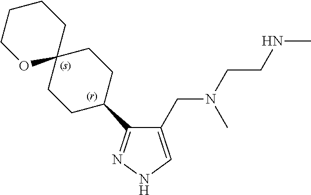 | 0.00733 | 0.02325 | 0.0162 | 0.00848 |

In certain embodiments, a provided compound inhibits an RMT (e.g., PRMT1, PRMT3, CARM1, PRMT6, and/or PRMT8). In certain embodiments, a provided compound inhibits wild-type PRMT1, PRMT3, CARM1, PRMT6, and/or PRMT8. In certain embodiments, a provided compound inhibits a mutant RMT. In certain embodiments, a provided compound inhibits PRMT1, PRMT3, CARM1, PRMT6, and/or PRMT8, e.g., as measured in an assay described herein. In certain embodiments, the RMT is from a human. In certain embodiments, a provided compound inhibits an RMT (e.g., PRMT1, PRMT3, CARM1, PRMT6, and/or PRMT8) at an IC₅₀ less than or equal to 10 μM. In certain embodiments, a provided compound inhibits an RMT (e.g., PRMT1, PRMT3, CARM1, PRMT6, and/or PRMT8) at an IC₅₀ less than or equal to 1 μM. In certain embodiments, a provided compound inhibits an RMT (e.g., PRMT1, PRMT3, CARM1, PRMT6, and/or PRMT8) at an IC₅₀ less than or equal to 0.1 μM. In certain embodiments, a provided compound inhibits an RMT (e.g., PRMT1, PRMT3, CARM1, PRMT6, and/or PRMT8) at an IC50 less than or equal to 0.01 μM. In certain embodiments, a provided compound inhibits an RMT (e.g., PRMT1, PRMT3, CARM1, PRMT6, and/or PRMT8) in a cell at an EC₃₀ less than or equal to 10 μM. In certain embodiments, a provided compound inhibits an RMT (e.g., PRMT1, PRMT3, CARM1, PRMT6, and/or PRMT8) in a cell at an EC₃₀ less than or equal to 12 μM. In certain embodiments, a provided compound inhibits an RMT (e.g., PRMT1, PRMT3, CARM1, PRMT6, and/or PRMT8) in a cell at an EC₃₀ less than or equal to 3 μM. In certain embodiments, a provided compound inhibits PRMT1 in a cell at an EC₃₀ less than or equal to 12 μM. In certain embodiments, a provided compound inhibits PRMT1 in a cell at an EC₃₀ less than or equal to 3 μM. In certain embodiments, a provided compound inhibits an RMT (e.g., PRMT1, PRMT3, CARM1, PRMT6, and/or PRMT8) in a cell at an EC₃₀ less than or equal to 1 μM. In certain embodiments, a provided compound inhibits an RMT (e.g., PRMT1, PRMT3, CARM1, PRMT6, and/or PRMT8) in a cell at an EC₃₀ less than or equal to 0.1 μM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM.

It will be understood by one of ordinary skill in the art that the RMT can be wild-type, or any mutant or variant.

The present disclosure provides pharmaceutical compositions comprising a compound described herein, e.g., a compound of Formula (S-I), or a pharmaceutically acceptable salt thereof, as described herein, and optionally a pharmaceutically acceptable excipient. It will be understood by one of ordinary skill in the art that the compounds described herein, or salts thereof, may be present in various forms, such as amorphous, hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting an RMT (e.g., PRMT1, PRMT3, CARM1, PRMT6, and/or PRMT8). In certain embodiments, the effective amount is an amount effective for treating an RMT-mediated disorder (e.g., a PRMT1-, PRMT3-, CARM1-, PRMT6-, and/or PRMT8-mediated disorder). In certain embodiments, the effective amount is a prophylactically effective amount. In certain embodiments, the effective amount is an amount effective to prevent an RMT-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

In some embodiments, a pharmaceutical composition described herein is sterilized.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Formula (S-I). In certain embodiments, the additional therapeutically active agent is not a compound of Formula (S-I). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, an additional therapeutically active agent is prednisolone, dexamethasone, doxorubicin, vincristine, mafosfamide, cisplatin, carboplatin, Ara-C, rituximab, azacitadine, panobinostat, vorinostat, everolimus, rapamycin, ATRA (all-trans retinoic acid), daunorubicin, decitabine, Vidaza, mitoxantrone, or IBET-151.

Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Compounds and compositions described herein are generally useful for the inhibition of RMT (e.g., PRMT1, PRMT3, CARM1, PRMT6, and/or PRMT8). In some embodiments, methods of treating an RMT-mediated disorder in a subject are provided which comprise administering an effective amount of a compound described herein (e.g., a compound of Formula (S-I)), or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a RMT-mediated disorder. In certain embodiments, the subject is susceptible to a RMT-mediated disorder.

As used herein, the term “RMT-mediated disorder” means any disease, disorder, or other pathological condition in which an RMT (e.g., PRMT1, PRMT3, CARM1, PRMT6, and/or PRMT8) is known to play a role. Accordingly, in some embodiments, the present disclosure relates to treating or lessening the severity of one or more diseases in which an RMT is known to play a role.

In some embodiments, the present disclosure provides a method of inhibiting an RMT comprising contacting the RMT with an effective amount of a compound described herein (e.g., a compound of Formula (S-I)), or a pharmaceutically acceptable salt thereof. The RMT may be purified or crude, and may be present in a cell, tissue, or subject. Thus, such methods encompass both inhibition of in vitro and in vivo RMT activity. In certain embodiments, the method is an in vitro method, e.g., such as an assay method. It will be understood by one of ordinary skill in the art that inhibition of an RMT does not necessarily require that all of the RMT be occupied by an inhibitor at once. Exemplary levels of inhibition of an RMT (e.g., PRMT1, PRMT3, CARM1, PRMT6, and/or PRMT8) include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In some embodiments, provided is a method of inhibiting RMT activity in a subject in need thereof (e.g., a subject diagnosed as having an RMT-mediated disorder) comprising administering to the subject an effective amount of a compound described herein (e.g., a compound of Formula (S-I)), or a pharmaceutically acceptable salt thereof, or a pharmaceutical composition thereof.

In certain embodiments, provided is a method of modulating gene expression in a cell which comprises contacting a cell with an effective amount of a compound of Formula (S-I), or a pharmaceutically acceptable salt thereof. In certain embodiments, the cell is in culture in vitro. In certain embodiments, the cell is in an animal, e.g., a human. In certain embodiments, the cell is in a subject in need of treatment.

In certain embodiments, provided is a method of modulating transcription in a cell which comprises contacting a cell with an effective amount of a compound of Formula (S-I), or a pharmaceutically acceptable salt thereof. In certain embodiments, the cell is in culture in vitro. In certain embodiments, the cell is in an animal, e.g., a human. In certain embodiments, the cell is in a subject in need of treatment.

In certain embodiments, a method is provided of selecting a therapy for a subject having a disease associated with an RMT-mediated disorder or mutation comprising the steps of determining the presence of an RMT-mediated disorder or gene mutation in an RMT gene (e.g., a PRMT1, PRMT3, CARM1, PRMT6, and/or PRMT8 gene) or and selecting, based on the presence of an RMT-mediated disorder a gene mutation in the RMT gene a therapy that includes the administration of a provided compound. In certain embodiments, the disease is cancer.

In certain embodiments, a method of treatment is provided for a subject in need thereof comprising the steps of determining the presence of an RMT-mediated disorder or a gene mutation in the RMT gene and treating the subject in need thereof, based on the presence of a RMT-mediated disorder or gene mutation in the RMT gene with a therapy that includes the administration of a provided compound. In certain embodiments, the subject is a cancer patient.

In some embodiments, a compound provided herein is useful in treating a proliferative disorder, such as cancer. For example, while not being bound to any particular mechanism, protein arginine methylation by PRMTs is a modification that has been implicated in signal transduction, gene transcription, DNA repair and mRNA splicing, among others; and overexpression of PRMTs within these pathways is often associated with various cancers. Thus, compounds which inhibit the action of PRMTs, as provided herein, are effective in the treatment of cancer.

In some embodiments, compounds provided herein are effective in treating cancer through the inhibition of PRMT1. For example, PRMT1 overexpression has been observed in various human cancers, including, but not limited to, breast cancer, prostate cancer, lung cancer, colon cancer, bladder cancer, and leukemia. In one example, PRMT1 specifically deposits an asymmetric dimethylarginine (aDMA) mark on histone H4 at arginine 3 (H4R3me2a), and this mark is associated with transcription activation. In prostate cancer, the methylation status of H4R3 positively correlates with increasing tumor grade and can be used to predict the risk of prostate cancer recurrence (Seligson et al., Nature 2005 435, 1262-1266). Thus, in some embodiments, inhibitors of PRMT1, as described herein, are useful in treating cancers associated with the methylation status of H4R3, e.g., prostate cancer. Additionally, the methylarginine effector molecule TDRD3 interacts with the H4R3me2a mark, and overexpression of TDRD3 is linked to poor prognosis for the survival of patients with breast cancer (Nagahata et al., Cancer Sci. 2004 95, 218-225). Thus, in some embodiments, inhibitors of PRMT1, as described herein, are useful in treating cancers associated with overexpression of TDRD3, e.g., breast cancer, as inhibition of PRMT1 leads to a decrease in methylation of H4R3, thereby preventing the association of overexpressed TDRD3 with H4R3me2a. In other examples, PRMT1 is known to have non-histone substrates. For example, PRMT1, when localized to the cytoplasm, methylates proteins that are involved in signal transduction pathways, e.g., the estrogen receptor (ER). The expression status of ER in breast cancer is critical for prognosis of the disease, and both genomic and non-genomic ER pathways have been implicated in the pathogenesis of breast cancer. For example, it has been shown that PRMT1 methylates ERα, and that ERα methylation is required for the assembly of ERα with SRC (a proto-oncogene tyrosine-protein kinase) and focal adhesion kinase (FAK). Further, the silencing of endogenous PRMT1 resulted in the inability of estrogen to activate AKT. These results suggested that PRMT1-mediated ERα methylation is required for the activation of the SRC-PI3K-FAK cascade and AKT, coordinating cell proliferation and survival. Thus, hypermethylation of ERα in breast cancer is thought to cause hyperactivation of this signaling pathway, providing a selective survival advantage to tumor cells (Le Romancer et al., Mol. Cell 2008 31, 212-221; Le Romancer et al., Steroids 2010 75, 560-564). Accordingly, in some embodiments, inhibitors of PRMT1, as described herein, are useful in treating cancers associated with ERα methylation, e.g., breast cancer. In yet another example, PRMT1 has been shown to be involved in the regulation of leukemia development. For example, SRC-associated in mitosis 68 kDa protein (SAM68; also known as KHDRBS1) is a well-characterized PRMT1 substrate, and when either SAM68 or PRMT1 is fused directly to the myeloid/lymphoid leukemia (MLL) gene, these fusion proteins can activate MLL oncogenic properties, implying that the methylation of SAM68 by PRMT1 is a critical signal for the development of leukemia (Cheung et al., Nature Cell Biol. 2007 9, 1208-1215). Accordingly, in some embodiments, inhibitors of PRMT1, as described herein, are useful in treating cancers associated with SAM68 methylation, e.g., leukemia. In still another example, PRMT1 is implicated in leukemia development through its interaction with AE9a, a splice isoform of AML1-ETO (Shia et al., Blood 2012 119:4953-62). Knockdown of PRMT1 affects expression of certain AE9a-activated genes and suppresses AE9a's self-renewal capability. It has also been shown that AE9a recruits PRMT1 to AE9a activated gene promoters, which leads to increased H4 Arg3 methylation, H3 Lys9/14 acetylation, and transcription activated. Accordingly, in some embodiments, inhibitors of PRMT1, as described herein, are useful in treating cancers associated with AML1-ETO, e.g., leukemia. Thus, without being bound by any particular mechanism, the inhibition of PRMT1, e.g., by compounds described herein, is beneficial in the treatment of cancer.

In some embodiments, compounds provided herein are effective in treating cancer through the inhibition of PRMT3. In one example, the DAL1 tumor suppressor protein has been shown to interact with PRMT3 and inhibits its methyltransferase activity (Singh et al., Oncogene 2004 23, 7761-7771). Epigenetic downregulation of DAL1 has been reported in several cancers (e.g., meningiomas and breast cancer), thus PRMT3 is expected to display increased activity, and cancers that display DAL1 silencing may, in some aspects, be good targets for PRMT3 inhibitors, e.g., those described herein. Thus, without being bound by any particular mechanism, the inhibition of PRMT3, e.g., by compounds described herein, is beneficial in the treatment of cancer.

In some embodiments, compounds provided herein are effective in treating cancer through the inhibition of PRMT4, also known as CARM1. For example, PRMT4 levels have been shown to be elevated in castration-resistant prostate cancer (CRPC), as well as in aggressive breast tumors (Hong et al., Cancer 2004 101, 83-89; Majumder et al., Prostate 2006 66, 1292-1301). Thus, in some embodiments, inhibitors of PRMT4, as described herein, are useful in treating cancers associated with PRMT4 overexpression. PRMT4 has also been shown to affect ERα-dependent breast cancer cell differentiation and proliferation (Al-Dhaheri et al., Cancer Res. 2011 71, 2118-2128), thus in some aspects PRMT4 inhibitors, as described herein, are useful in treating ERα-dependent breast cancer by inhibiting cell differentiation and proliferation. In another example, PRMT4 has been shown to be recruited to the promoter of E2F1 (which encodes a cell cycle regulator) as a transcriptional co-activator (Frietze et al., Cancer Res. 2008 68, 301-306). Thus, PRMT4-mediated upregulation of E2F1 expression may contribute to cancer progression and chemoresistance as increased abundance of E2F1 triggers invasion and metastasis by activating growth receptor signaling pathways, which in turn promote an antiapoptotic tumor environment (Engelmann and Pützer, Cancer Res 2012 72; 571). Accordingly, in some embodiments, the inhibition of PRMT4, e.g., by compounds provided herein, is useful in treating cancers associated with E2F1 upregulation. Thus, without being bound by any particular mechanism, the inhibition of PRMT4, e.g., by compounds described herein, is beneficial in the treatment of cancer.

In some embodiments, compounds provided herein are effective in treating cancer through the inhibition of PRMT6. For example, PRMT6 has been reported to be overexpressed in a number of cancers, e.g., bladder and lung cancer (Yoshimatsu et al., Int. J. Cancer 2011 128, 562-573). Thus, in some embodiments, the inhibition of PRMT6, by compounds provided herein, is useful in treating cancers associated with PRMT6 overexpression. In some aspects, PRMT6 is primarily thought to function as a transcriptional repressor, although it has also been reported that PRMT6 functions as a co-activator of nuclear receptors. For example, as a transcriptional repressor, PRMT6 suppresses the expression of thrombospondin 1 (TSP1; also known as THBS1; a potent natural inhibitor of angiogenesis and endothelial cell migration) and p21 (a natural inhibitor of cyclin dependent kinase), thereby contributing to cancer development and progression (Michaud-Levesque and Richard, J. Biol. Chem. 2009 284, 21338-21346; Kleinschmidt et al., PLoS ONE 2012 7, e41446). Accordingly, in some embodiments, the inhibition of PRMT6, by compounds provided herein, is useful in treating cancer by preventing the repression of THBs1 and/or p21. Thus, without being bound by any particular mechanism, the inhibition of PRMT6, e.g., by compounds described herein, is beneficial in the treatment of cancer.

In some embodiments, compounds provided herein are effective in treating cancer through the inhibition of PRMT8. For example, deep-sequencing efforts of cancer genomes (e.g., COSMIC) have revealed that of all the PRMTs, PRMT8 is reported to be the most mutated. Of 106 sequenced genomes, 15 carry mutations in the PRMT8 coding region, and nine of these result in an amino acid change (Forbes et al., Nucleic Acids Res. 2011 39, D945-D950). Because of its high rate of mutation in cancer, PRMT8 is thought to contribute to the initiation or progression of cancer. Thus, without being bound by any particular mechanism, the inhibition of PRMT8, e.g., by compounds described herein, is beneficial in the treatment of cancer.

In some embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, a compound provided herein is useful in treating diseases associated with increased levels of circulating asymmetric dimethylarginine (aDMA), e.g., cardiovascular disease, diabetes, kidney failure, renal disease, pulmonary disease, etc. Circulating aDMA is produced by the proteolysis of asymmetrically dimethylated proteins. PRMTs which mediate aDMA methylation include, e.g., PRMT1, PRMT3, PRMT4, PRMT6, and PRMT8. aDMA levels are directly involved in various diseases as aDMA is an endogenous competitive inhibitor of nitric oxide synthase (NOS), thereby reducing the production of nitric oxide (NO) (Vallance et al., J. Cardiovasc. Pharmacol. 1992 20 (Suppl. 12):S60-2). NO functions as a potent vasodilator in endothelial vessels, and as such inhibiting its production has major consequences on the cardiovascular system. For example, since PRMT1 is a major enzyme that generates aDMA, the dysregulation of its activity is likely to regulate cardiovascular diseases (Boger et al., Ann. Med. 2006 38:126-36), and other pathophysiological conditions such as diabetes mellitus (Sydow et al., Vasc. Med. 2005 10 (Suppl. 1):S35-43), kidney failure (Vallance et al., Lancet 1992 339:572-5), and chronic pulmonary diseases (Zakrzewicz et al., BMC Pulm. Med. 2009 9:5). Additionally, it has been demonstrated that the expression of PRMT1 and PRMT3 are increased in coronary heart disease (Chen et al., Basic Res. Cardiol. 2006 101:346-53). In another example, aDMA elevation is seen in patients with renal failure, due to impaired clearance of this metabolite from the circulation (Jacobi et al., Am. J. Nephrol. 2008 28:224-37). Thus, circulating aDMA levels is observed in many pathophysiological situations. Accordingly, without being bound by any particular mechanism, the inhibition of PRMTs, e.g., by compounds described herein, results in the decrease of circulating aDMA, which is beneficial in the treatment of diseases associated with increased levels of circulating aDMA, e.g., cardiovascular disease, diabetes, kidney failure, renal disease, pulmonary disease, etc. In certain embodiments, a compound described herein is useful for treating or preventing vascular diseases.

In some embodiments, a compound provided herein is useful in treating metabolic disorders. For example, PRMT1 has been shown to enhance mRNA levels of FoxO1 target genes in gluconeogenesis, which results in increased hepatic glucose production, and knockdown of PRMT promotes inhibition of FoxO1 activity and thus inhibition of hepatic gluconeogenesis (Choi et al., Hepatology 2012 56:1546-56). Additionally, genetic haploinsufficiency of Prmt1 has been shown to reduce blood glucose levels in mouse models. Thus, without being bound by any particular mechanism, the inhibition of PRMT1, e.g., by compounds described herein, is beneficial in the treating of metabolic disorders, such as diabetes. In some embodiments, a provided compound is useful in treating type I diabetes. In some embodiments, a provided compound is useful in treating type II diabetes.

In some embodiments, a compound provided herein is useful in treating muscular dystrophies. For example, PRMT1, as well as PRMT3 and PRMT6, methylate the nuclear poly(A)-binding protein (PABPN1) in a region located near its C-terminus (Perreault et al., J. Biol. Chem. 2007 282:7552-62). This domain is involved in the aggregation of the PABPN1 protein, and abnormal aggregation of this protein is involved in the disease oculopharyngeal muscular dystrophy (Davies et al., Int. J. Biochem. Cell. Biol. 2006 38:1457-62). Thus, without being bound by any particular mechanism, the inhibition of PRMTs, e.g., by compounds described herein, is beneficial in the treatment of muscular dystrophies, e.g., oculopharyngeal muscular dystrophy, by decreasing the amount of methylation of PABPN1, thereby decreasing the amount of PABPN1 aggregation.

CARM1 is also the most abundant PRMT expressed in skeletal muscle cells, and has been found to selectively control the pathways modulating glycogen metabolism, and associated AMPK (AMP-activated protein kinase) and p38 MAPK (mitogen-activated protein kinase) expression. See, e.g., Wang et al., Biochem (2012) 444:323-331. Thus, in some embodiments, inhibitors of CARM1, as described herein, are useful in treating metabolic disorders, e.g., for example skeletal muscle metabolic disorders, e.g., glycogen and glucose metabolic disorders. Exemplary skeletal muscle metabolic disorders include, but are not limited to, Acid Maltase Deficiency (Glycogenosis type 2; Pompe disease), Debrancher deficiency (Glycogenosis type 3), Phosphorylase deficiency (McArdle's; GSD 5), X-linked syndrome (GSD9D), Autosomal recessive syndrome (GSD9B), Tarui's disease (Glycogen storage disease VII; GSD 7), Phosphoglycerate Mutase deficiency (Glycogen storage disease X; GSDX; GSD 10), Lactate dehydrogenase A deficiency (GSD 11), Branching enzyme deficiency (GSD 4), Aldolase A (muscle) deficiency, β-Enolase deficiency, Triosephosphate isomerase (TIM) deficiency, Lafora's disease (Progressive myoclonic epilepsy 2), Glycogen storage disease (Muscle, Type 0, Phosphoglucomutase 1 Deficiency (GSD 14)), and Glycogenin Deficiency (GSD 15).

In some embodiments, a compound provided herein is useful in treating autoimmune disease. For example, several lines of evidence strongly suggest that PRMT inhibitors may be valuable for the treatment of autoimmune diseases, e.g., rheumatoid arthritis. PRMTs are known to modify and regulate several critical immunomodulatory proteins. For example, post-translational modifications (e.g., arginine methylation), within T cell receptor signaling cascades allow T lymphocytes to initiate a rapid and appropriate immune response to pathogens. Co-engagement of the CD28 costimulatory receptor with the T cell receptor elevates PRMT activity and cellular protein arginine methylation, including methylation of the guanine nucleotide exchange factor Vav1 (Blanchet et al., J. Exp. Med. 2005 202:371-377). PRMT inhibitors are thus expected to diminish methylation of the guanine exchange factor Vav1, resulting in diminished IL-2 production. In agreement, siRNA directed against PRMT5 was shown to both inhibit NFAT-driven promoter activity and IL-2 secretion (Richard et al., Biochem J. 2005 388:379-386). In another example, PRMT1 is known to cooperate with PRMT4 to enhance NFkB p65-driven transcription and facilitate the transcription of p65 target genes like TNFα (Covic et al., Embo. J. 2005 24:85-96). Thus, in some embodiments, PRMT1 and/or PRMT4 inhibitors, e.g., those described herein, are useful in treating autoimmune disease by decreasing the transcription of p65 target genes like TNFα. These examples demonstrate an important role for arginine methylation in inflammation. Thus, without being bound by any particular mechanism, the inhibition of PRMTs, e.g., by compounds described herein, is beneficial in the treatment of autoimmune diseases.

In some embodiments, a compound provided herein is useful in treating neurological disorders, such as amyotrophic lateral sclerosis (ALS). For example, a gene involved in ALS, TLS/FUS, often contains mutated arginines in certain familial forms of this disease (Kwiatkowski et al., Science 2009 323:1205-8). These mutants are retained in the cytoplasm, which is similar to reports documenting the role arginine methylation plays in nuclear-cytoplasmic shuffling (Shen et al., Genes Dev. 1998 12:679-91). This implicates PRMT, e.g., PRMT1, function in this disease, as it was demonstrated that TLS/FUS is methylated on at least 20 arginine residues (Rappsilber et al., Anal. Chem. 2003 75:3107-14). Thus, in some embodiments, the inhibition of PRMTs, e.g., by compounds provided herein, are useful in treating ALS by decreasing the amount of TLS/FUS arginine methylation.

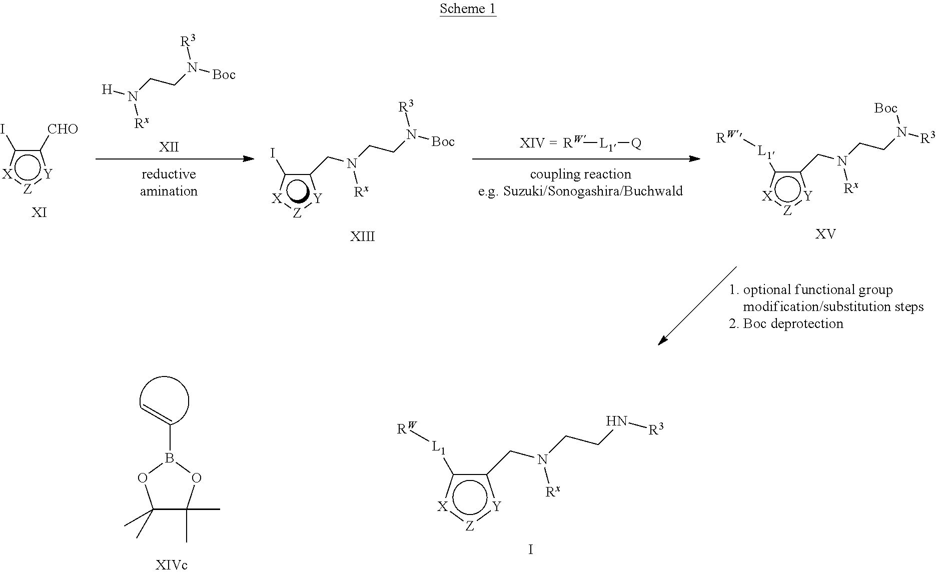

Scheme 1 shows an exemplary general synthesis route to pyrazole compounds of formula I, wherein R^{W′} is either the same as R^{W} or is precursor of R^{W} and L_{1′} is either the same as L₁ or is a precursor of L₁ and R^{W}, L₁, R^{x}, R³, X, Y and Z are as defined above. In the first step iodopyrazole carboxaldehydes of general formula XI are allowed to react with mono-Boc protected ethylenediamines XII under reductive amination conditions (e.g. sodium cyanoborohydride and catalytic acid such as acetic acid) in an appropriate solvent such as methanol to give intermediates of general formula XIII. In certain embodiments, Sonagashira reaction of intermediates of general formula XIII with boronic acids or boronic esters of general formula XIV in which L₁ is an acetylene linker and Q is a boronic acid or boronic ester group in the presence of a palladium catalyst (e.g. PdCl₂(dppf)) and a base (e.g. potassium carbonate) in an organic solvent (e.g. toluene) at elevated temperature yields intermediates of general formula XV-a in which L_{1′} is an acetylene linker. Boc deprotection of intermediates of general formula XV-a gives acetylene compounds of formula VI-a. In certain embodiments, Suzuki reaction of intermediates of general formula XIII with boronic acids or boronic esters of general formula XIV in which L_{1′} is a trans-olefin linker and Q is a boronic acid or boronic ester group in the presence of a palladium catalyst (e.g. PdCl₂(dppf)) and a base (e.g. potassium carbonate) in an organic solvent (e.g. toluene) at elevated temperature yields intermediates of general formula XV-b in which L_{1′} is an olefin linker. Boc deprotection of intermediates of general formula XV-b gives olefin compounds of formula VI-b. In certain embodiments, Suzuki reaction of intermediates of general formula XIII with pinacol boranes of general formula XIVc in which L_{1′} is bond, R^{W}′ is a heterocycloalkenyl or cycloalkenyl group and Q is a pinacol borane group yields intermediates of general formula XV-c in which L_{1′} is bond and R^{W}′ is a heterocycloalkenyl or cycloalkenyl group. In certain embodiments, compounds of formula I wherein L₁ is bond and R^{W} is a heterocyclyl or carbocyclyl group can be prepared by hydrogenation of intermediates of formula XV-c followed by Boc deprotection. In certain embodiments, compounds of formula I where L₁ is —O— can be synthesized from intermediates of general formula XIII by Goldberg reaction with alcohols of formula R^{W}OH followed by Boc deprotection. In certain embodiments, compounds of formula I where L₁ is —N(R^{B})— can be synthesized from intermediates of general formula XIII by palladium catalyzed Buchwald coupling reaction conditions with amines of formula R^{W}N(R^{B})H followed by Boc deprotection. In certain embodiments, compounds of formula I where L₁ is —C(═O)NR^{B}— can be synthesized from intermediates of general formula XIII under known copper catalyzed coupling reaction conditions of amides with aryliodides using copper iodide an amine ligand and a base with amides of formula R^{W}C(═O)NHR^{B} followed by Boc deprotection.

Scheme 1.1 shows an alternative general synthesis route to pyrazole compounds of Formula (S-I), that involves reversal in the order of the first two steps of the reaction sequence detailed for Scheme 1.0. Thus, in the first step iodopyrazole carboxaldehydes of general formula XI are coupled with compounds or reagents of general formula XIV (e.g. via Suzuki reaction with pinacol boranes of general formula XIVc in which L_{1′} is bond, R^{W}′ is a heterocycloalkenyl or cycloalkenyl group and Q is a pinacol borane group) and in a second step the corresponding reductive amination reaction to yield common intermediates of general formula XV is a carried out.

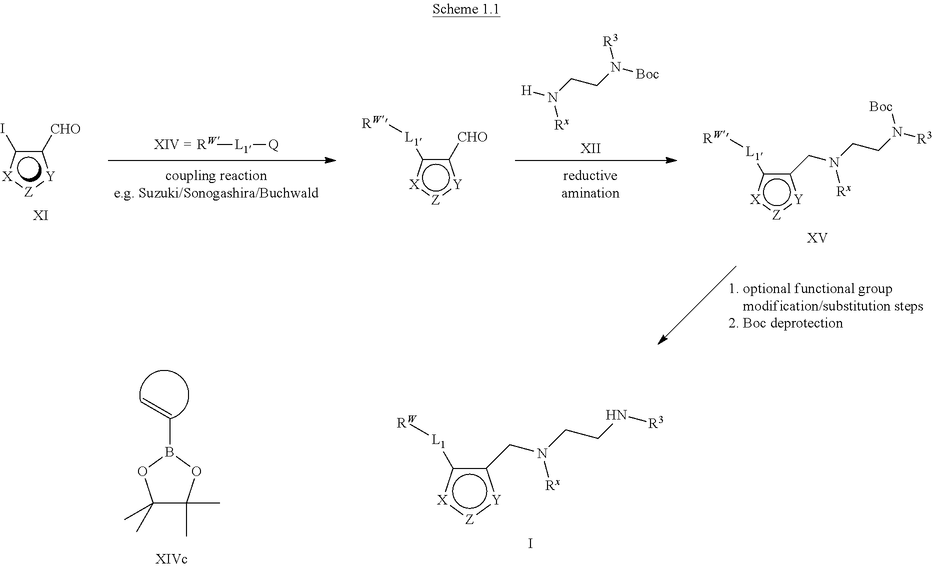

In certain embodiments, iodopyrazole carboxaldehydes of general formula XI may be prepared from suitable known pyrazole compound intermediates by established synthetic chemistry methods. Standard methods include direct iodination of a pyrazole 3-carboxylate and Sandmeyer reaction of a 3-amino pyrazole 4-carboxylate. In certain embodiments, iodopyrazole carboxaldehydes can be derived from iodopyrazole carboxylates by reduction to a hydroxymethyl group followed by oxidation to carboxaldehyde. In certain embodiments, mono-Boc protected ethylenediamines XII can be synthesized by standard methods known in the literature for derivatizing or preparing ethylenediamines. For example intermediates of formula XII may be prepared by treatment of the corresponding unprotected diamine precursors with Boc₂O and purifying the mixture of mono and dibocylated products. In certain embodiments, pyrazole compounds of general formula II can be prepared from iodopyrazole carboxaldehydes of general formula XXI as depicted in Scheme 2. In certain embodiments where R⁴ is hydrogen compounds of general formula II are equivalent to compounds of general formula III which are tautomers. In certain embodiments, R^{4′} is a protecting group such as tetrahydropyranyl (THP) which maybe cleaved to hydrogen under acidic conditions in the final Boc-deprotection step. In certain embodiments, iodopyrazole carboxaldehydes of general formula XXI can be prepared as depicted in Scheme 3.

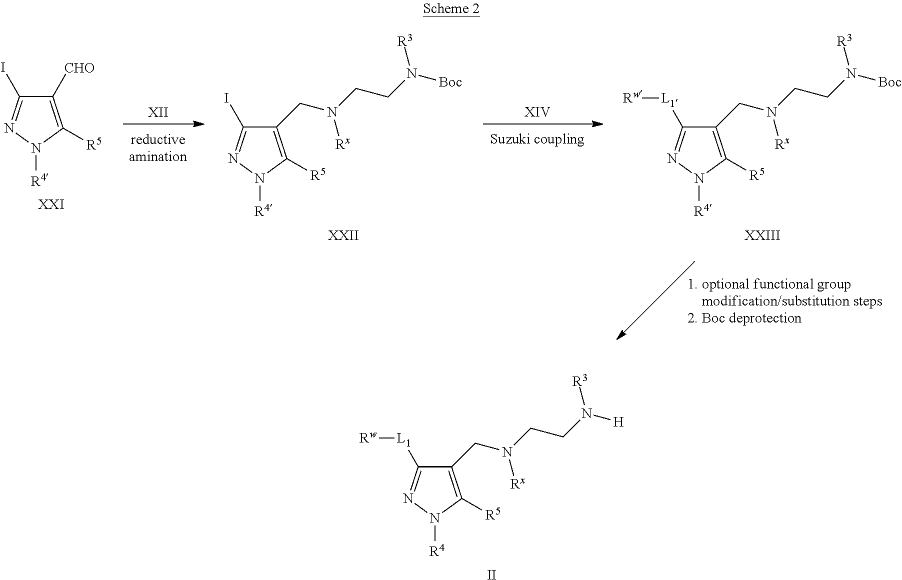

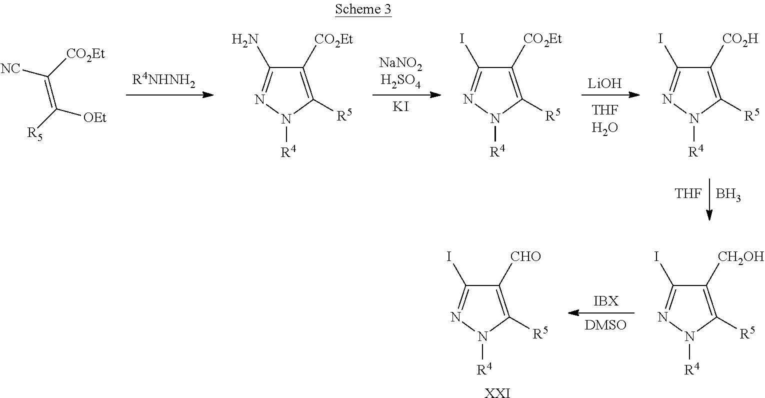

In certain embodiments, iodopyrazole carboxaldehydes of general formula XXI can be prepared as depicted in Scheme 4 which also provides iodopyrazole carboxyaldehydes of general formula XXXI. In certain embodiments, alkylation of intermediates of general formula XXX gives a mixture of pyrazole nitrogen alkylated isomers which are separated by chromatography to give pure isomers XXI and XXXI. In certain embodiments, pyrazole compounds of general formula III can be prepared from iodopyrazole carboxaldehydes of general formula XXXI as depicted in Scheme 5.

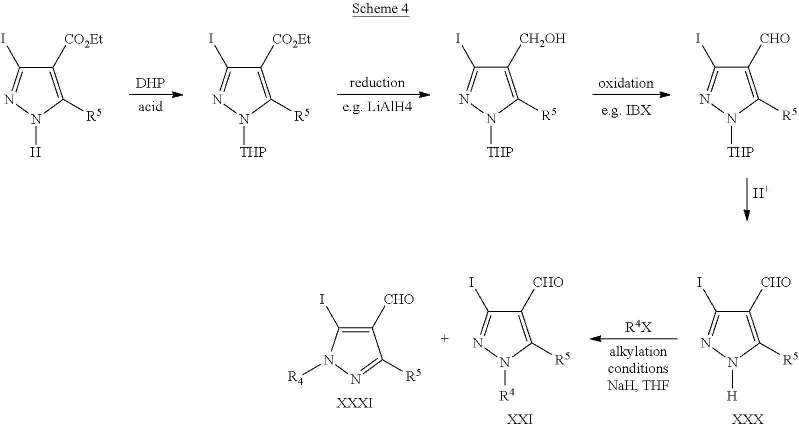

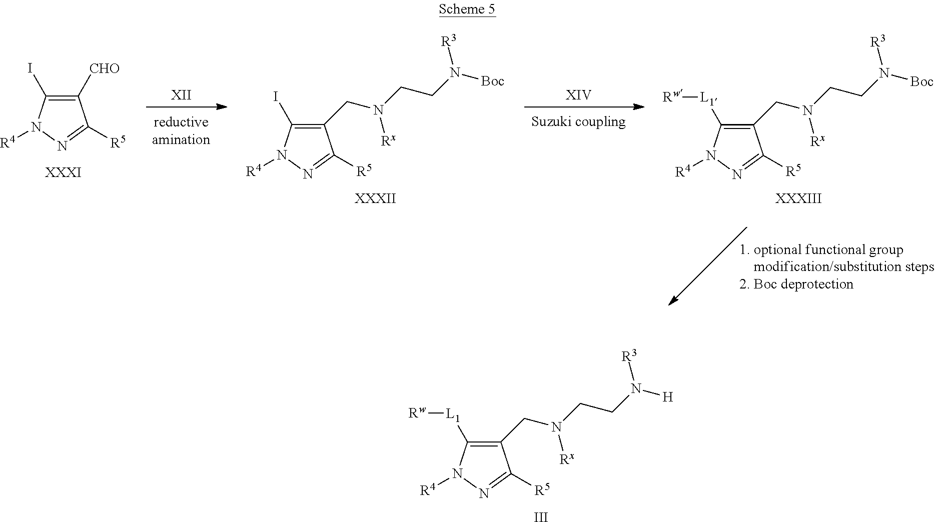

In certain embodiments, pyrazole compounds of general formula IV can be prepared from iodopyrazole carboxaldehydes of general formula XLI as depicted in Scheme 6. In certain embodiments where R⁴ is hydrogen compounds of general formula IV are equivalent to compounds of general formula V which are tautomers. In certain embodiments where R⁴ in compounds of formula IV is hydrogen, R^{4′} in intermediate XLI may be a selected protecting group such as tetrahydropyranyl (THP) which can be cleaved to hydrogen under acidic conditions in the final Boc-deprotection step.

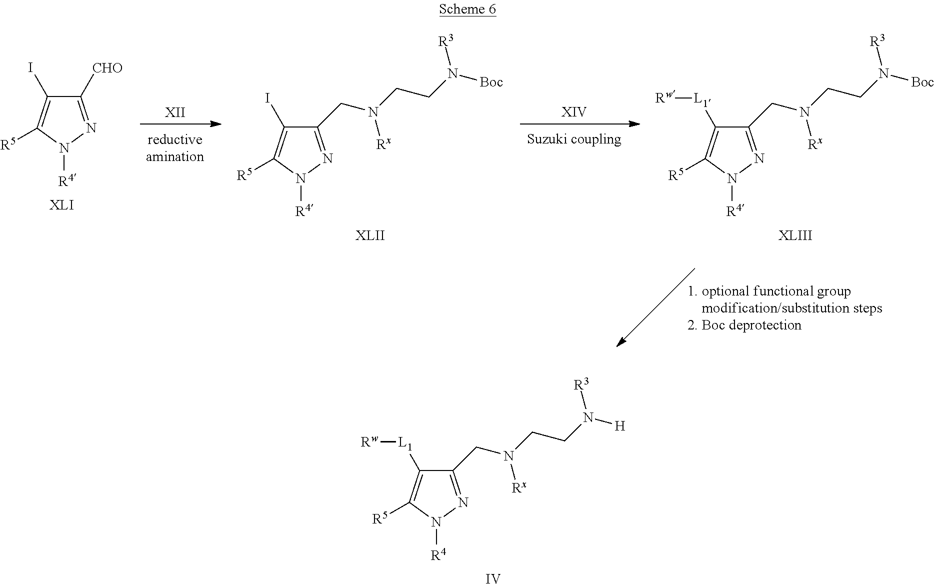

In certain embodiments, iodopyrazole carboxaldehydes of general formula XLI and LI can be prepared as depicted in Scheme 7. In certain embodiments, an R⁴ group of iodopyrazole carboxaldehydes may be introduced by alkylation of intermediates of formula XLVII. This reaction can give a mixture of intermediate compounds of formulas XLI and LI which may be separated by chromatography. In certain embodiments, THP protected intermediates of formula XLVI can be used to prepare compounds of formula IV where R⁴═H as also depicted in Scheme 7.

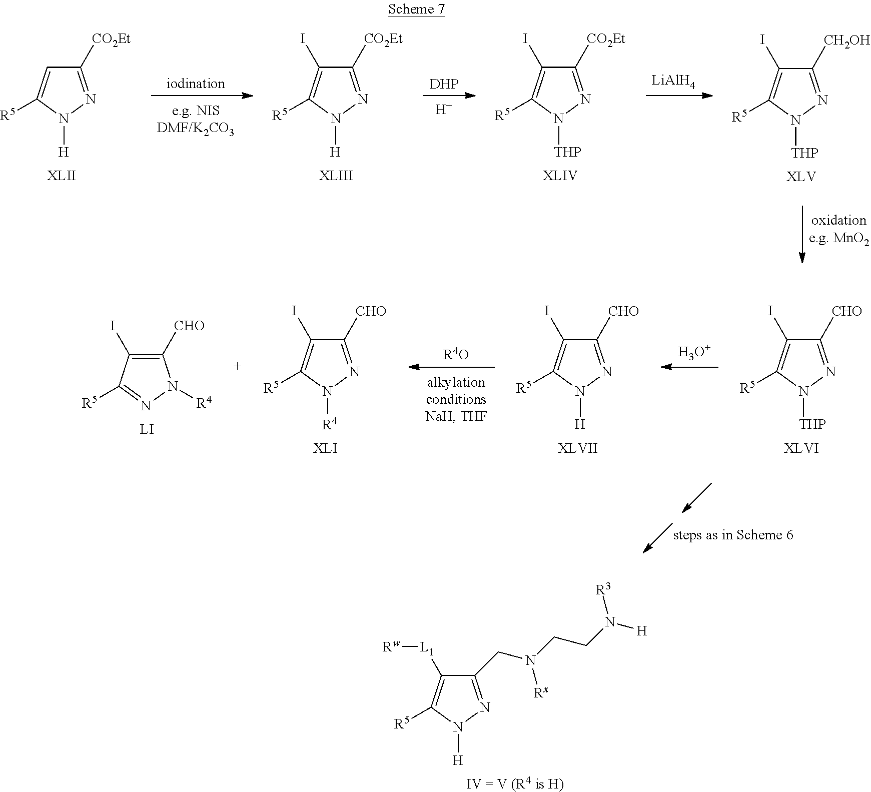

In certain embodiments, pyrazole compounds of general formula V can be prepared from iodopyrazole carboxaldehydes of general formula LI as depicted in Scheme 8.

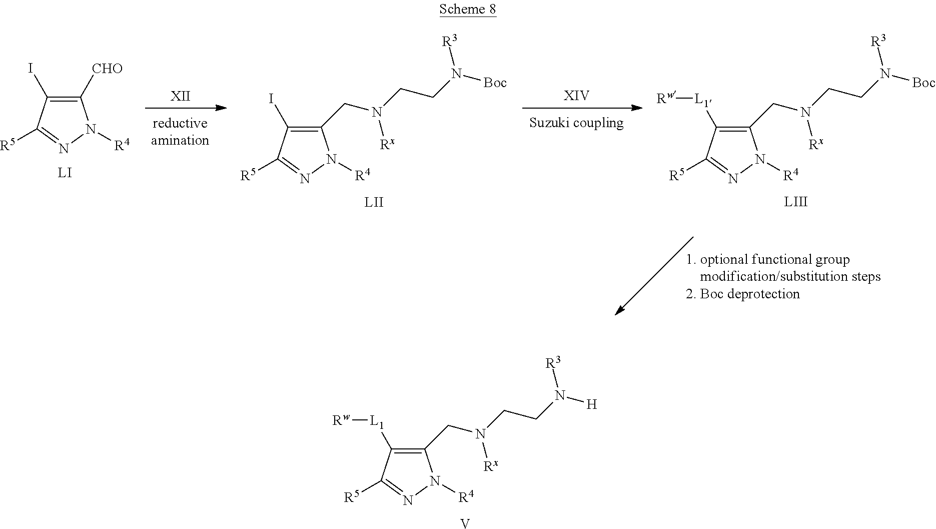

In certain embodiments, boronic acids or esters of general formula XIVa, XIVb and XIVc are commercially available. In certain embodiments, compounds of general formula XIVa, and XIVb can also be prepared from alkenyl bromides and terminal alkynes using standard methods such as treatment with n-BuLi followed by trapping the intermediate lithium species with trimethylborate. In certain embodiments, compounds of general formula XIVc can be prepared from the corresponding cyclic ketones LX via intermediate enol triflates as depicted in Scheme 9.

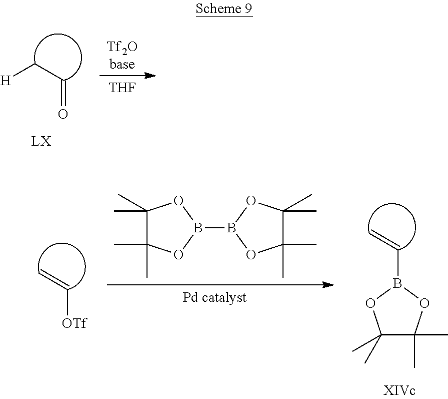

### EXAMPLES

In order that the invention described herein may be more fully understood, the following examples are set forth. It should be understood that these examples are for illustrative purposes only and are not to be construed as limiting this invention in any manner.

### Synthetic Methods

General methods and experimental procedures for preparing and characterizing compounds of the present invention are set forth below. Wherever needed, reactions were heated using conventional hotplate apparatus or heating mantle or microwave irradiation equipment. Reactions were conducted with or without stirring, under atmospheric or elevated pressure in either open or closed vessels. Reaction progress was monitored using conventional techniques such as TLC, HPLC, UPLC, or LCMS using instrumentation and methods described below. Reactions were quenched and crude compounds isolated using conventional methods as described in the specific examples provided. Solvent removal was carried out with or without heating, under atmospheric or reduced pressure, using either a rotary or centrifugal evaporator.

Compound purification was carried out as needed using a variety of traditional methods including, but not limited to, preparative chromatography under acidic, neutral, or basic conditions using either normal phase or reverse phase HPLC or flash columns or Prep-TLC plates. Compound purity and mass confirmations were conducted using standard HPLC and/or UPLC and/or MS spectrometers and/or LCMS and/or GC equipment (e.g., including, but not limited to the following instrumentation: Waters Alliance 2695 with 2996 PDA detector connected with ZQ detector and ESI source; Shimadzu LDMS-2020; Waters Acquity H Class with PDA detector connected with SQ detector and ESI source; Agilent 1100 Series with PDA detector; Waters Alliance 2695 with 2998 PDA detector; AB SCIEX API 2000 with ESI source; Agilent 7890 GC). Exemplified compounds were dissolved in either MeOH or MeCN to a concentration of approximately 1 mg/mL and analyzed by injection of 0.5-10 μL into an appropriate LCMS system using the methods provided in the following table:

| | | | | | | MS | |
|---|---|---|---|---|---|---|---|
| | | | | | | Heat | MS |
| | | | | Flow | | Block | Detector |
| | | Mobile | Mobile | Rate | | Temp | Voltage |
| Method | Column | Phase A | Phase B | (mL/min) | Gradient Profile | (° C.) | (kV) |
| A | Shim-pack | Water/0.05% | ACN/0.05% | 1 | 5% to 100% B in 2.0 | 250 | 1.5 |
| | XR-ODS | TFA | TFA | | minutes, 100% B for | | |
| | 2.2 μm | | | | 1.1 minutes, 100% to | | |
| | 3.0 × 50 mm | | | | 5% B in 0.2 minutes, | | |
| | | | | | then stop | | |
| B | Gemini-NX | Water/ | ACN | 1 | 5% to 100% B in 2.0 | 200 | 0.75 |
| | 3 μm C18 | 0.04% | | | minutes, 100% B for | | |
| | 110A | Ammonia | | | 1.1 minutes, 100% to | | |
| | | | | | 5% B in 0.1 minutes, | | |
| | | | | | then stop | | |
| C | Shim-pack | Water/0.05% | ACN/0.05% | 1 | 5% to 100% B in 2.0 | 250 | 0.85 |
| | XR-ODS | FA | FA | | minutes, 100% B for | | |
| | 1.6 μm | | | | 1.1 minutes, 100% to | | |
| | 2.0 × 50 mm | | | | 5% B in 0.1 minutes, | | |
| | | | | | then stop | | |
| D | Shim-pack | Water/0.05% | ACN/0.05% | 1 | 5% to 100% B in 2.0 | 250 | 0.95 |
| | XR-ODS | TFA | TFA | | minutes, 100% B for | | |
| | 2.2 μm | | | | 1.1 minutes, 100% to | | |
| | 3.0 × 50 mm | | | | 5% B in 0.1 minutes, | | |
| | | | | | then stop | | |
| E | Waters | Water/0.05% | ACN/0.05% | 0.9 | 5% to 100% B in 2.0 | 250 | 1.5 |
| | Xselect C18 | FA | FA | | minutes, 100% B for | | |
| | 3.5 μm | | | | 1.2 minutes, 100% to | | |
| | 3.0 × 50 mm | | | | 5% B in 0.1 minutes, | | |
| | | | | | then stop | | |
| F | Shim-pack | Water/0.05% | ACN/0.05% | 1 | 5% to 80% B in 3.25 | 200 | 0.95 |
| | XR-ODS | TFA | TFA | | minutes, 80% B for | | |
| | 2.2 μm | | | | 1.35 minutes, 80% to | | |
| | 3.0 × 50 mm | | | | 5% B in 0.3 minutes, | | |
| | | | | | then stop | | |
| G | Shim-pack | Water/0.05% | ACN/0.05% | 1 | 5% to 70% B in 2.50 | 200 | 0.95 |
| | XR-ODS | TFA | TFA | | minutes, 70% B for | | |
| | 2.2 μm | | | | 0.70 minutes, 70% to | | |
| | 3.0 × 50 mm | | | | 5% B in 0.1 minutes, | | |
| | | | | | then stop | | |
| H | Shim-pack | Water/0.05% | ACN/0.05% | 1 | 5% to 100% B in 2.20 | 250 | 0.95 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| I | Shim-pack | Water/0.05% | ACN/0.05% | 1 | 5% to 100% B in 1.20 | 250 | 0.95 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| J | Shim-pack | Water/0.05% | ACN/0.05% | 1 | 5% to 70% B in 3.20 | 250 | 0.95 |
| | XR-ODS | TFA | TFA | | minutes, 70% B for | | |
| | 2.2 μm | | | | 0.75 minutes, 70% to | | |
| | 3.0 × 50 mm | | | | 5% B in 0.35 minutes, | | |
| | | | | | then stop | | |
| K | Shim-pack | Water/0.05% | ACN/0.05% | 1 | 5% to 80% B in 3.00 | 250 | 1.5 |
| | XR-ODS | TFA | TFA | | minutes, 80% B for 0.8 | | |
| | 2.2 μm | | | | minutes, 80% to 5% B | | |
| | 3.0 × 50 mm | | | | in 0.1 minutes, then | | |
| | | | | | stop | | |
| L | Shim-pack | Water/0.05% | ACN/0.05% | 1 | 5% to 100% B in 3.00 | 250 | 1.5 |
| | XR-ODS | TFA | TFA | | minutes, 100% B for | | |
| | 2.2 μm | | | | 0.8 minutes, 100% to | | |
| | 3.0 × 50 mm | | | | 5% B in 0.1 minutes, | | |
| | | | | | then stop | | |
| M | Shim-pack | Water/0.05% | ACN/0.05% | 1 | 5% to 100% B in 2.20 | 250 | 1.5 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| N | Shim-pack | Water/0.05% | ACN/0.05% | 1 | 5% to 80% B in 2.20 | 250 | 1.5 |
| | XR-ODS | TFA | TFA | | minutes, 80% B for 1.0 | | |
| | 2.2 μm | | | | minutes, 80% to 5% B | | |
| | 3.0 × 50 mm | | | | in 0.1 minutes, then | | |
| | | | | | stop | | |
| O | Zorbax | Water/0.05% | ACN/0.05% | 1 | 5% to 70% B in 8.00 | 250 | 1.5 |
| | Eclipse Plus | TFA | TFA | | minutes, 70% B for 2.0 | | |
| | C18 | | | | minutes, then stop | | |
| | 4.6 × 100 mm | | | | | | |
| P | Shim-pack | Water/0.05% | ACN/0.05% | 1 | 5% to 65% B in 3.00 | 250 | 1.5 |
| | XR-ODS | TFA | TFA | | minutes, 65% B for | | |
| | 2.2 μm | | | | 0.80 minutes, 100% to | | |
| | 3.0 × 50 mm | | | | 5% B in 0.1 minutes, | | |
| | | | | | then stop | | |
| Q | Shim-pack | Water/0.05% | ACN/0.05% | 1 | 5% to 60% B in 2.50 | 250 | 0.95 |
| | XR-ODS | TFA | TFA | | minutes, 60% B for 0.7 | | |
| | 2.2 μm | | | | minutes, 60% to 5% B | | |
| | 3.0 × 50 mm | | | | in 0.1 minutes, then | | |
| | | | | | stop | | |
| R | Shim-pack | Water/0.05% | ACN/0.05% | 1 | 5% to 50% B in 2.50 | 250 | 0.95 |
| | XR-ODS | TFA | TFA | | minutes, 50% B for 0.7 | | |
| | 2.2 μm | | | | minutes, 50% to 5% B | | |
| | 3.0 × 50 mm | | | | in 0.1 minutes, then | | |
| | | | | | stop | | |
| S | XBridge | Water/0.05% | ACN/0.05% | 1 | 5% to 95% B in 2.20 | 250 | 0.9 |
| | C18 3.5 μm | TFA | TFA | | minutes, 95% B for | | |
| | 3.0 × 50 mm | | | | 1.00 minutes, 95% to | | |
| | | | | | 5% B in 0.1 minutes, | | |
| | | | | | then stop | | |
| T | Shim-pack | Water/0.05% | ACN/0.05% | 0.7 | 5% to 100% B in 2.0 | 250 | 0.85 |
| | XR-ODS | FA | FA | | minutes, 100% B for | | |
| | 1.6 μm | | | | 1.1 minutes, 100% to | | |
| | 2.0 × 50 mm | | | | 5% B in 0.1 minutes, | | |
| | | | | | then stop | | |
| U | Shim-pack | Water/0.05% | ACN/0.05% | 1 | 5% to 40% B in 2.50 | 250 | 0.95 |
| | XR-ODS | TFA | TFA | | minutes, 40% B for 0.7 | | |
| | 2.2 μm | | | | minutes, 40% to 5% B | | |
| | 3.0 × 50 mm | | | | in 0.1 minutes, then | | |
| | | | | | stop | | |
| V | Shim-pack | Water/0.05% | ACN/0.05% | 1 | 5% to 60% B in 4.20 | 200 | 1.05 |
| | XR-ODS | TFA | TFA | | minutes, 60% B for 1.0 | | |
| | 2.2 μm | | | | minutes, 60% to 5% B | | |
| | 3.0 × 50 mm | | | | in 0.1 minutes, then | | |
| | | | | | stop | | |
| W | Shim-pack | Water/0.05% | ACN/0.05% | 1 | 5% to 100% B in 2.20 | 200 | 0.95 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| X | Shim-pack | Water/0.05% | ACN/0.05% | 0.7 | 5% to 100% B in 2.0 | 200 | 0.85 |
| | XR-ODS | FA | FA | | minutes, 100% B for | | |
| | 1.6 μm | | | | 1.1 minutes, 100% to | | |
| | 2.0 × 50 mm | | | | 5% B in 0.1 minutes, | | |
| | | | | | then stop | | |
| Y | Ecliplis Plus | Water/0.05% | ACN | 1 | 5% to 100% B in 2.0 | 250 | 1 |
| | C18 3.5 μm | TFA | | | minutes, 100% B for | | |
| | 4.6 × 50 mm | | | | 1.0 minutes, 100% to | | |
| | | | | | 5% B in 0.1 minutes, | | |
| | | | | | then stop | | |
| Z | Ecliplis Plus | Water/10 mM | ACN/5% | 1 | 5% to 100% B in 2.0 | 250 | 1.1 |
| | C18 3.5 μm | ammonium | water | | minutes, 100% B for | | |
| | 4.6 × 50 mm | carbonate | | | 1.0 minutes, 100% to | | |
| | | | | | 5% B in 0.1 minutes, | | |
| | | | | | then stop | | |
| A1 | Shim-pack | Water/0.05% | ACN | 1 | 5% to 100% B in 2.0 | 250 | 1 |
| | XR-ODS | TFA | | | minutes, 100% B for | | |
| | 2.2 μm | | | | 1.0 minutes, 100% to | | |
| | 3.0 × 50 mm | | | | 5% B in 0.1 minutes, | | |
| | | | | | then stop | | |
| A2 | Ecliplis Plus | Water/10 mM | ACN | 1 | 5% to 100% B in 2.0 | 250 | 0.95 |
| | C18 3.5 μm | ammonium | | | minutes, 100% B for | | |
| | 4.6 × 50 mm | acetate | | | 1.4 minutes, 100% to | | |
| | | | | | 5% B in 0.1 minutes, | | |
| | | | | | then stop | | |
| A3 | Acquity | Water/5 mM | ACN/0.1% | 0.55 | 5% B at 0.01 min up to | | |
| | BEH C18 | ammonium | FA | | 0.4 min, 35% B at 0.8 min, | | |
| | 1.7 μm 2.1 × | acetate/ | | | 55% B at 1.2 min, | | |
| | 50 mm | 0.1% FA | | | 100% B in 1.3 minutes, | | |
| | | | | | at 2.5 min up to 3.30 min, | | |
| | | | | | 5% B at 3.31 min | | |
| | | | | | up to 4.0 min, then | | |
| | | | | | stop | | |
| A4 | Shim-pack | Water/0.05% | ACN/0.05% | 1 | 5% to 30% B in 8.0 | 250 | 1.5 |
| | XR-ODS | TFA | TFA | | minutes, 30% B for 2.0 | | |
| | 3.0 × 50 mm | | | | minutes, then stop | | |
| A5 | Shim-pack | Water/0.05% | ACN/0.05% | 1 | 5% to 100% B in 2.2 | 250 | 1.5 |
| | XR-ODS | TFA | TFA | | minutes, 100% B for | | |
| | 3.0 × 50 mm | | | | 1.0 minutes, 100% to | | |
| | | | | | 5% B in 0.1 minutes, | | |
| | | | | | then stop | | |
| A6 | Atlantis | Water/0.05% | ACN/0.05% | 0.8 | 95% to 60% B in 4.0 | 250 | 1.5 |
| | HILIC | TFA | TFA | | minutes, 60% B for 4.0 | | |
| | 3.0 × 100 mm | | | | minutes, then stop | | |
| A7 | Shim-pack | Water/0.05% | ACN/0.05% | 1 | 5% B for 0.5 minutes, | 250 | 1.5 |
| | XR-ODS | TFA | TFA | | 5% to 75% B at 2.2 | | |
| | 3.0 × 50 mm | | | | minutes, 100% B for | | |
| | | | | | 1.0 minutes, 100% to | | |
| | | | | | 5% B in 0.1 minutes, | | |
| | | | | | then stop | | |
| A8 | Zorbax SB- | Water/0.05% | ACN/0.05% | 1.2 | 5% to 70% B in 10.0 | 250 | 1.05 |
| | C18 | TFA | TFA | | minutes, 70% B for 5.0 | | |
| | 5 μm | | | | minutes, then stop | | |
| | 4.6 × 150 mm | | | | | | |
| A9 | Shim-pack | Water/0.05% | ACN/0.05% | 1 | 5% to 40% B in 4.4 | 250 | 0.95 |
| | XR-ODS | TFA | TFA | | minutes, 40% B for 0.9 | | |
| | 3.0 × 50 mm | | | | minutes, then stop | | |
| A10 | Atlantis T3 | Water/0.05% | ACN/0.05% | 1 | 5% to 50% B in 8.0 | 200 | 1.05 |
| | 3 μm | TFA | TFA | | minutes, 50% B for 2.0 | | |
| | 4.6 × 100 mm | | | | minutes, then stop | | |
| A11 | Shim-pack | Water/0.05% | ACN/0.05% | 1 | 5% B for 0.5 minutes, | 250 | 1.50 |
| | XR-ODS | TFA | TFA | | 5% to 100% B in 1.7 | | |
| | 3.0 × 50 mm | | | | minutes, 100% B for | | |
| | | | | | 1.0 minute, 100% to | | |
| | | | | | 5% B in 0.1 minute, | | |
| | | | | | then stop | | |

Compound structure confirmations were carried out using standard 300 or 400 MHz NMR spectrometers with NOe's conducted whenever necessary.

The following abbreviations are used herein:

| Abbreviation | Meaning |
|---|---|
| ACN | acetonitrile |
| atm. | atmosphere |
| DCM | dichloromethane |
| DHP | dihydropyran |
| DIBAL | diisobutyl aluminum hydride |
| DIEA | diisopropyl ethylamine |
| DMF | dimethyl formamide |
| DMF-DMA | dimethyl formamide dimethyl acetal |
| DMSO | dimethyl sulfoxide |
| dppf | 1,1′-bis(diphenylphosphino)ferrocene |
| EA | ethyl acetate |
| ESI | electrospray ionization |
| EtOH | ethanol |
| FA | formic acid |
| GC | gas chromatography |
| h | hour |
| Hex | hexanes |
| HMDS | hexamethyl disilazide |
| HPLC | high performance liquid chromatography |
| IPA | isopropanol |
| LCMS | liquid chromatography/mass spectrometry |
| MeOH | methanol |
| min | minutes |
| NBS | N-bromo succinimide |
| NCS | N-chloro succinimide |
| NIS | N-iodo succinimide |
| NMR | nuclear magnetic resonance |
| NOe | nuclear Overhauser effect |
| Prep. | Preparative |
| PTSA | para-toluene sulfonic acid |
| Rf | retardation factor |
| rt | room temperature |
| RT | retention time |
| sat. | Saturated |
| SGC | silica gel chromatography |
| TBAF | tetrabutyl ammonium fluoride |
| TEA | Triethylamine |
| TFA | trifluoroacetic acid |
| THF | Tetrahydrofuran |
| TLC | thin layer chromatography |
| UPLC | ultra performance liquid chromatography |
| LiHMDS | lithium hexamethyldisilazide |
| TMAD | tetramethyl azocarboxamide |

### Intermediate Synthesis

### Example A-1. Synthesis of Intermediate tert-butyl (2-(((3-iodo-1-(tetrahydro-2H-pyran-2-yl)-1H-pyrazol-4-yl)methyl)(methyl)amino)ethyl)carbamate

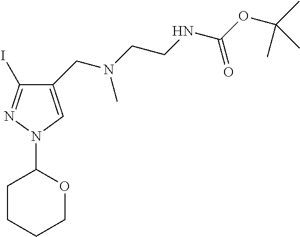 [SMILES: CN(CCNC(=O)OC(C)(C)C)Cc1cn(C2CCCCO2)nc1I]

### Step 1: tert-butyl (2-(((3-iodo-1-(tetrahydro-2H-pyran-2-yl)-1H-pyrazol-4-yl)methyl)(methyl)amino)ethyl)carbamate

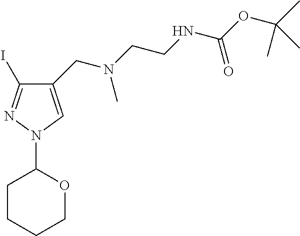 [SMILES: CN(CCNC(=O)OC(C)(C)C)Cc1cn(C2CCCCO2)nc1I]

A mixture of 3-iodo-1-(oxan-2-yl)-1H-pyrazole-4-carbaldehyde (3.2 g, 10.45 mmol, 1.00 equiv), tert-butyl N-[2-(methylamino)ethyl]carbamate (2.2 g, 12.63 mmol, 1.21 equiv) and NaBH(OAc)₃ (6.65 g, 31.38 mmol, 3.00 equiv) in dichloroethane (30 mL) was stirred for 2 h at room temperature. The reaction was quenched with 50 mL of saturated aqueous sodium bicarbonate solution. The resulting mixture was extracted with 3×200 mL of dichloromethane. The combined organic layers was dried over anhydrous sodium sulfate and concentrated under vacuum. The residue was purified on a silica gel column eluted with 30-100% ethyl acetate in petroleum ether to give 4.05 g (83%) of tert-butyl (2-(((3-iodo-1-(tetrahydro-2H-pyran-2-yl)-1H-pyrazol-4-yl)methyl)(methyl)amino)ethyl)carbamate as a light yellow oil. ¹H-NMR (300 MHz, CDCl₃): δ 7.48 (s, 1H), 5.35-5.30 (m, 1H), 4.13-4.03 (m, 1H), 3.71-3.63 (m, 1H), 3.36 (s, 2H), 3.26-3.25 (m, 2H), 2.52-2.49 (m, 2H), 2.21 (s, 3H), 2.09-2.01 (m, 3H), 1.68-1.58 (m, 3H), 1.44 (s, 9H) ppm. LCMS (method C, ESI): RT=0.58 min, m/z=465.0 [M+H]⁺.

### Example A-2. Synthesis of Intermediate tert-butyl (2-(((3-iodo-1-(tetrahydro-2H-pyran-2-yl)-1H-pyrazol-4-yl)methyl)(methyl)amino)ethyl)(methyl)carbamate

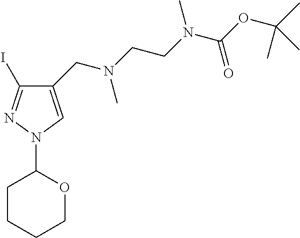 [SMILES: CN(CCN(C)C(=O)OC(C)(C)C)Cc1cn(C2CCCCO2)nc1I]

### Step 1: Ethyl 3-iodo-1H-pyrazole-4-carboxylate

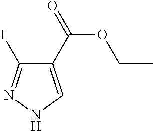

To a stirred solution of ethyl 3-amino-1H-pyrazole-4-carboxylate (10 g, 64.45 mmol, 1.00 equiv) in 50% sulfuric acid (90 mL) at 5° C. was added dropwise a solution of NaNO₂ (7.4 g, 107.25 mmol, 1.66 equiv) in water (15 mL). The reaction was stirred at 5° C. for another 30 min. A solution of KI (32.1 g, 193.37 mmol, 3.00 equiv) in water (15 mL) was added dropwise at 5° C. The reaction was allowed to stir at 5° C. for 1 h and then quenched by the addition of 50 mL of water. The precipitate was collected by filtration and then dissolved in 150 mL of ethyl acetate. The resulting solution was washed sequentially with 1×100 mL of saturated Na₂SO₃ solution, 1×100 mL of saturated sodium bicarbonate solution and 1×100 mL of brine. The organic layer was dried over anhydrous sodium sulfate and concentrated under vacuum to give 10.8 g (63%) of ethyl 3-iodo-1H-pyrazole-4-carboxylate as a yellow solid. ¹H-NMR (300 MHz, CDCl₃): δ 8.18 (s, 1H), 4.38-4.29 (m, 2H), 1.41-1.33 (m, 3H) ppm. LCMS (method B, ESI): RT=1.36 min, m/z=267.0 [M+H]⁺.

### Step 2: Ethyl 3-iodo-1-(tetrahydro-2H-pyran-2-yl)-1H-pyrazole-4-carboxylate

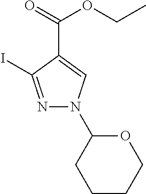 [SMILES: CCOC(=O)c1cn(C2CCCCO2)nc1I]

A solution of ethyl 3-iodo-1H-pyrazole-4-carboxylate (10.8 g, 40.60 mmol, 1.00 equiv), 3,4-dihydro-2H-pyran (10 g, 118.88 mmol, 2.93 equiv) and TsOH (780 mg, 4.53 mmol, 0.11 equiv) in THF (100 mL) was stirred for 2 h at 60° C. The reaction mixture was cooled to room temperature and quenched by the addition of 100 mL of saturated sodium bicarbonate solution. The resulting solution was extracted with 2×80 mL of dichloromethane. The combined organic layers was dried over anhydrous sodium sulfate and concentrated under vacuum. The residue was purified on a silica gel column eluted with ethyl acetate/petroleum ether (1:20) to give 13 g (91%) of ethyl 3-iodo-1-(oxan-2-yl)-1H-pyrazole-4-carboxylate as a yellow oil. ¹H-NMR (400 MHz, CDCl₃): δ 8.04 (s, 1H), 5.40-5.38 (m, 1H), 4.34-4.29 (m, 2H), 4.08-4.05 (m, 1H), 3.73-3.70 (m, 1H), 2.07-1.98 (m, 3H), 1.69-1.62 (m, 3H), 1.39-1.32 (m, 3H) ppm. LCMS (method C, ESI): RT=1.53 min, m/z=351.0 [M+H]⁺.

### Step 3: 3-Iodo-1-(tetrahydro-2H-pyran-2-yl)-1H-pyrazole-4-carboxylic acid

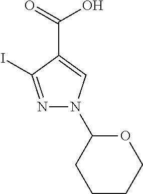 [SMILES: O=C(O)c1cn(C2CCCCO2)nc1I]

To a solution of ethyl 3-iodo-1-(oxan-2-yl)-1H-pyrazole-4-carboxylate (85 g, 242.75 mmol, 1.00 equiv) in THF (300 mL) and methanol (300 mL) was added a solution of LiOH (17.5 g, 730.69 mmol, 3.01 equiv) in water (400 mL). The resulting solution was stirred at room temperature overnight and then concentrated under vacuum to remove the organic solvent. The resulting solution was diluted with 400 mL of H₂O and then acidified to pH 6.0 with 1M hydrochloric acid. The mixture was extracted with 3×800 mL of dichloromethane. The combined organic layers was washed with 3×1000 mL of brine, dried over anhydrous sodium sulfate and concentrated under vacuum to give 75 g (96%) of 3-iodo-1-(oxan-2-yl)-1H-pyrazole-4-carboxylic acid as an off-white solid. LCMS (method D, ESI): RT=1.23 min, m/z=323.0 [M+H]⁺.

### Step 4: (3-iodo-1-(tetrahydro-2H-pyran-2-yl)-1H-pyrazol-4-yl)methanol

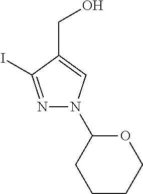 [SMILES: OCc1cn(C2CCCCO2)nc1I]

To a solution of 3-iodo-1-(oxan-2-yl)-1H-pyrazole-4-carboxylic acid (28 g, 86.93 mmol, 1.00 equiv) in anhydrous THF (300 mL) maintained under nitrogen at 5° C. was added a 1M solution of BH₃ in THF (300 mL) dropwise with stirring. The reaction was stirred overnight at room temperature and then quenched by the addition of 300 mL of saturated NH₄Cl solution. The resulting mixture was extracted with 3×1000 mL of dichloromethane. The combined organic layers was dried over anhydrous sodium sulfate and concentrated under vacuum. The residue was purified on a silica gel column eluted with ethyl acetate/petroleum ether (1:1) to give 12.67 g (47%) of (3-iodo-1-(oxan-2-yl)-1H-pyrazol-4-yl)methanol as a white solid. ¹H-NMR (400 MHz, DMSO-d6): δ 7.73 (s, 1H), 5.37-5.34 (m, 1H), 4.92 (s, 1H), 4.20 (d, J=3.6 Hz, 2H), 3.89-3.88 (m, 1H), 3.65-3.57 (m, 1H), 2.09-2.00 (m, 1H), 1.99-1.90 (m, 2H), 1.69-1.61 (m, 1H), 1.49-1.46 (m, 2H) ppm. LCMS (method A, ESI): RT=1.16 min, m/z=309.0 [M+H]⁺.

### Step 5: 3-Iodo-1-(tetrahydro-2H-pyran-2-yl)-1H-pyrazole-4-carbaldehyde

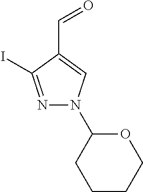 [SMILES: O=Cc1cn(C2CCCCO2)nc1I]

Into a 250-mL 3-necked round-bottom flask purged and. To a stirred solution of oxalyl chloride (18.576 g, 146.35 mmol, 3.01 equiv) in anhydrous dichloromethane (300 mL) maintained under nitrogen at −78° C. was added DMSO (15.138 g, 193.75 mmol, 3.98 equiv) dropwise. The reaction mixture was stirred at −65° C. for 30 min. A solution of (3-iodo-1-(oxan-2-yl)-1H-pyrazol-4-yl)methanol (15.0 g, 48.68 mmol, 1.00 equiv) in dichloromethane (100 mL) was then added dropwise at −65° C. and the reaction was stirred for another 60 min at −65° C. Triethylamine (40.6 mL) was added dropwise at −65° C. and the reaction was stirred for 30 min at −65° C. The reaction was warmed to 0° C. then quenched by the addition of 100 mL of saturated NH₄Cl solution. The resulting mixture was extracted with 3×400 mL of dichloromethane. The combined organic layers was dried over anhydrous sodium sulfate and concentrated under vacuum. The residue was purified on a silica gel column eluted with ethyl acetate/petroleum ether (1:20) to give 13.48 g (90%) of 3-iodo-1-(oxan-2-yl)-1H-pyrazole-4-carbaldehyde as a golden oil. ¹H-NMR (300 MHz, DMSO-d6): δ 9.69 (s, 1H), 8.57 (s, 1H), 5.49 (dd, J=2.7 Hz, 9.9 Hz, 1H), 3.95-3.91 (m, 1H), 3.68-3.62 (m, 1H), 2.11-2.01 (m, 3H), 1.69-1.62 (m, 3H) ppm. LCMS (method A, ESI): RT=1.35 min, m/z=307.0 [M+H]⁺.

### Step 6: tert-Butyl (2-(((3-iodo-1-(tetrahydro-2H-pyran-2-yl)-1H-pyrazol-4-yl)methyl)(methyl)amino)ethyl)(methyl)carbamate

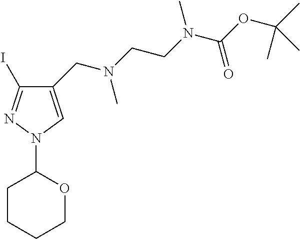 [SMILES: CN(CCN(C)C(=O)OC(C)(C)C)Cc1cn(C2CCCCO2)nc1I]

A mixture of 3-iodo-1-(oxan-2-yl)-1H-pyrazole-4-carbaldehyde (21.5 g, 70.24 mmol, 1.00 equiv), tert-butyl N-methyl-N-(2-(methylamino)ethyl)carbamate (20 g, 106.23 mmol, 1.51 equiv) and NaBH(OAc)₃ (29.8 g, 137.98 mmol, 1.96 equiv) in dichloroethane (300 mL) was stirred for 1 h at room temperature. The reaction was diluted with 300 mL of dichloromethane and then washed with 3×300 mL of brine. The organic layer was dried over anhydrous sodium sulfate and concentrated under vacuum. The residue was purified on a silica gel column eluted with 0-7% methanol in dichloromethane to give 31 g (92%) of tert-butyl (2-(((3-iodo-1-(tetrahydro-2H-pyran-2-yl)-1H-pyrazol-4-yl)methyl)(methyl)amino)ethyl)(methyl)carbamate as a yellow oil. ¹H-NMR (300 MHz, CDCl₃): δ 7.62 (s, 1H), 5.34-5.30 (m, 1H), 4.06-4.02 (m, 1H), 3.68-3.62 (m, 1H), 3.42-3.38 (m, 4H), 2.85 (s, 4H), 2.62-2.53 (m, 2H), 2.47-2.46 (m, 2H), 2.13-1.97 (m, 3H), 1.74-1.69 (m, 3H), 1.46 (s, 9H) ppm. LCMS (method A, ESI): RT=1.17 min, m/z=479.0 [M+H]⁺.

### Exemplified Compound Synthesis

### Example B-1. Synthesis of ({3-[(3R)-4,4-dimethyl-3-(2-methylpropoxy)cyclohexyl]-1H-pyrazol-4-yl}methyl)(methyl)[2-(methylamino)ethyl]amine (Compound 20)

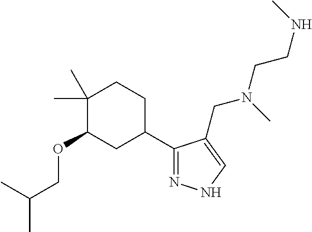 [SMILES: CNCCN(C)Cc1c[nH]nc1C1CCC(C)(C)[C@H](OCC(C)C)C1]

### Step-1: Synthesis of (1S,6S)-5,5-dimethyl-7-oxabicyclo[4.1.0]heptan-2-one

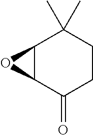

(1S,2S)-1,2-diphenylethane-1,2-diamine (3.42 g, 16.11 mmol) and trifluoroacetic acid (1.2 ml, 16.11 mmol) were dissolved in dioxane (300 ml). The solution was stirred for 30 min before adding 4,4-dimethylcyclohex-2-en-1-one (10 g, 80.53 mmol) and hydrogen peroxide (10.58 ml, 120.79 mmol). The reaction was heated to 50° C. and monitored by NMR. The reaction was stirred for 72 h after which time the reaction was quenched with NH₄Cl (saturated 100 ml). The solution was extracted with DCM (4×100 ml). The combined organic layers were dried over Na₂SO₄ and evaporated to dryness to afford 12.5 g of (1S,6S)-5,5-dimethyl-7-oxabicyclo[4.1.0]heptan-2-one as an orange oil (100%, containing ˜10% dioxane w/w). The material was used without further purification. ¹H NMR (250 MHz, CDCl₃) δ: 3.23 (d, J=4.0 Hz, 1H), 3.17 (dd, J=4.0, 1.2 Hz, 1H), 2.41 (ddd, J=18.8, 6.5, 3.2 Hz, 1H), 2.19 (ddd, J=18.7, 11.5, 6.9 Hz, 1H), 1.90 (td, J=12.5, 11.5, 6.5 Hz, 1H), 1.35 (dtd, J=9.9, 3.1, 1.2 Hz, 1H), 1.22 (s, 3H), 1.06 (s, 3H).

### Step-2: Synthesis of (3R)-3-hydroxy-4,4-dimethylcyclohexan-1-one

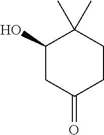

At rt under nitrogen, lithium (1.63 g, 235 mmol) was added to a solution of naphthalene (40.23 g, 314 mmol) in dry THF (600 ml). The solution quickly turned dark green and the reaction was stirred at rt until full dissolution of the lithium (˜5 h). The solution was cooled to −78° C. and a solution of (1S,6S)-5,5-dimethyl-7-oxabicyclo[4.1.0]heptan-2-one (11 g, 78.47 mmol) in dry THF (300 ml) was added. The reaction was stirred for 1 h. The reaction was quenched with water (30 ml) and allowed to warm to rt. Further 300 ml of water were added and the solution was extracted with Et₂O (2×500 ml). The combined organic layers were dried over Na₂SO₄ and evaporated to dryness. The residue was purified by Biotage (SNAP 340 g, eluent Hep/EtOAc/NEt₃ 90/10/1 to 10/90/1) to afford 5.81 g of (3R)-3-hydroxy-4,4-dimethylcyclohexan-1-one (52%) as an orange oil. ¹H NMR (500 MHz, CDCl₃) δ: 3.77-3.62 (m, 1H), 2.64 (ddd, J=14.9, 4.3, 1.0 Hz, 1H), 2.46-2.36 (m, 1H), 2.36-2.25 (m, 2H), 1.94-1.82 (m, 1H), 1.83-1.76 (m, 1H), 1.54-1.44 (m, 1H), 1.13 (s, 3H), 1.07 (s, 3H).

### Step-3: Synthesis of (3R)-3-[(tert-butyldimethylsilyl)oxy]-4,4-dimethylcyclohexan-1-one

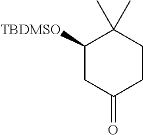 [SMILES: CC1(C)CCC(=O)C[C@H]1O[Si](C)(C)C(C)(C)C]

(3R)-3-hydroxy-4,4-dimethylcyclohexan-1-one (5.81 g, 40.86 mmol), tert-butyl(chloro)dimethylsilane (9.24 g, 61.29 mmol) and 1H-imidazole (6.95 g, 102.15 mmol) were dissolved in DMF (50 ml). The reaction was stirred at rt for 16 h. The reaction was quenched with saturated aqueous ammonium chloride (30 ml) and was extracted with EtOAc (3×30 ml); the combined organic layers were washed with water (30 ml), dried over Na₂SO₄ and evaporated to dryness. NMR showed DMF and TBS-OH present; the residue was co-evaporated with toluene (4×50 ml) until no more TBS-OH was detected by NMR to afford 8.4 g of (3R)-3-[(tert-butyldimethylsilyl)oxy]-4,4-dimethylcyclohexan-1-one as a yellow oil (80%). ¹H NMR (500 MHz, CDCl₃) δ: 3.64 (dd, J=7.4, 4.1 Hz, 1H), 2.63-2.49 (m, 1H), 2.39-2.25 (m, 3H), 1.95-1.78 (m, 1H), 1.43 (dt, J=13.8, 7.1 Hz, 1H), 1.07 (s, 3H), 1.01 (s, 3H), 0.88 (s, 9H), 0.04 (d, J=6.0 Hz, 6H).

### Step-4: Synthesis of (3R)-3-[(tert-butyldimethylsilyl)oxy]-4,4-dimethylcyclohex-1-en-1-yl trifluoromethanesulfonate

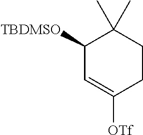 [SMILES: CC1(C)CCC(OS(=O)(=O)C(F)(F)F)=C[C@H]1O[Si](C)(C)C(C)(C)C]

(3R)-3-[(tert-butyldimethylsilyl)oxy]-4,4-dimethylcyclohexan-1-one (3 g, 11.7 mmol) was dissolved in dry THF (250 ml). The solution was cooled to −78° C. and 1M lithium 1,1,1,3,3,3-hexamethyldisilazan-2-ide (23.4 ml) was slowly added. The reaction was stirred for 45 min and a solution of N-(5-chloropyridin-2-yl)-1,1,1-trifluoro-N-[(trifluoromethyl)sulfonyl]methanesulfonamide (8.59 g, 21.88 mmol) in dry THF (60 ml) was slowly added. The reaction was allowed to warm to rt and stirred for 3 h. The reaction was quenched with NH₄Cl (saturated, 100 ml) and the solution was extracted with EtOAc (3×100 ml). The combined organic layers were dried over Na₂SO₄, evaporated to dryness and the residue was purified by Biotage (SNAP HP 100 g, eluent Hep/EtOAc 100/0 to 90/10) to afford 260 mg of (3R)-3-[(tert-butyldimethylsilyl)oxy]-4,4-dimethylcyclohex-1-en-1-yl trifluoromethanesulfonate (5%, mainly 1 isomer) and 2.85 g (56%, —1:1 mix of isomers). Both batches were combined for the next step. Fraction 1: ¹H NMR (500 MHz, CDCl₃) δ: 5.74-5.52 (m, 1H), 3.57 (t, J=5.3 Hz, 1H), 2.53 (dd, J=17.3, 2.2 Hz, 1H), 2.32-2.21 (m, 1H), 2.13 (ddt, J=17.6, 4.4, 2.5 Hz, 1H), 1.88 (ddt, J=17.5, 4.4, 2.4 Hz, 1H), 0.96-0.84 (m, 15H), 0.06 (d, J=7.4 Hz, 6H). Fraction 2: ¹H NMR (500 MHz, CDCl₃) δ: 5.76-5.52 (m, 1H), 4.02-3.52 (m, 1H), 2.63-2.08 (m, 3H), 1.99-1.66 (m, 1H), 1.56-1.41 (m, 1H), 0.97-0.81 (m, 15H), 0.06 (d, J=7.3 Hz, 6H).

### Step-5: Synthesis of tert-butyl({[(1R)-6,6-dimethyl-3-(tetramethyl-1,3,2-dioxaborolan-2-yl)cyclohex-2-en-1-yl]oxy}) dimethylsilane

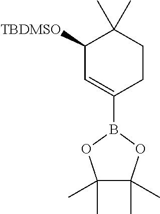 [SMILES: CC1(C)CCC(B2OC(C)(C)C(C)(C)O2)=C[C@H]1O[Si](C)(C)C(C)(C)C]

A suspension of 3-[(tert-butyldimethylsilyl)oxy]-4,4-dimethylcyclohex-1-en-1-yl trifluoromethane sulfonate] (90%, 3.11 g, 7.2 mmol), 4,4,4′,4′,5,5,5′,5′-octamethyl-2,2′-bi-1,3,2-dioxaborolane (2.74 g, 10.81 mmol) and potassium acetate (3.15 ml, 50.43 mmol) in 1,4-dioxane (100 ml) was degassed with a N₂ sparge for 10 min whilst stirring at rt. Pd(dppf)Cl₂ (0.59 g, 0.72 mmol) was added to this suspension and stirred at 90° C. for 3.5 h before allowing to cool to rt for 16 h. The reaction mixture was diluted with EtOAc (50 ml) and water (50 ml). The organic layer was separated and the aqueous was extracted with EtOAc (3×50 ml). The combined organic layers were dried over Na₂SO₄, filtered and evaporated in vacuo. Purification by silica gel column chromatography, on a Biotage Isolera system, using a 100 g HP-Sil SNAP cartridge, eluting with EtOAc:heptanes (0:100-5:95), afforded tert-butyl({[(1R)-6,6-dimethyl-3-(tetramethyl-1,3,2-dioxaborolan-2-yl)cyclohex-2-en-1-yl]oxy})dimethylsilane as an orange oil (2.02 g, 76%). ¹H NMR (500 MHz, CDCl₃) δ: 6.50-6.12 (m, 1H), 3.90-3.41 (m, 1H), 2.34-2.18 (m, 1H), 2.18-1.96 (m, 2H), 1.96-1.82 (m, 1H), 1.25 (d, J=3.2 Hz, 12H), 0.95-0.80 (m, 15H), 0.11-0.00 (m, 6H).

### Step-6: Synthesis of tert-butyl N-{2-[({3-[(3R)-3-[(tert-butyldimethylsilyl)oxy]-4,4-dimethylcyclohex-1-en-1-yl]-1-(oxan-2-yl)-1H-pyrazol-4-yl}methyl)(methyl) amino]ethyl}-N-methylcarbamate

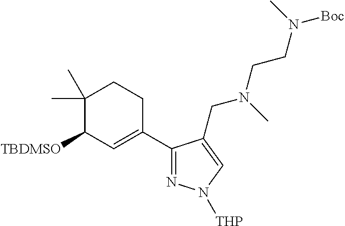

Tert-butyl({[(1R)-6,6-dimethyl-3-(tetramethyl-1,3,2-dioxaborolan-2-yl)cyclohex-2-en-1-yl]oxy})dimethyl silane (2 g, 5.46 mmol), tert-butylN-[2-({[3-iodo-1-(oxan-2-yl)-1H-pyrazol-4-yl]methyl}(methyl)amino) ethyl]-N-methylcarbamate (2.61 g, 5.46 mmol) and dipotassium carbonate (2.26 g, 16.37 mmol) were suspended in dioxane/water (240 ml, 7/1). The solution was degassed with nitrogen for 10 min and Pd(dppf)Cl₂ (0.45 g, 0.55 mmol) was added. The reaction was heated to 100° C. for 16 h. The solvents were evaporated. The residue was purified by Biotage (SNAP HP 100 g, eluent Hep/EtOAc (+1% NEt₃) 95/5 to 60/40) to afford 2.5 g of desired tert-butyl N-{2-[({3-[(3R)-3-[(tert-butyldimethylsilyl)oxy]-4,4-dimethylcyclohex-1-en-1-yl]-1-(oxan-2-yl)-1H-pyrazol-4-yl}methyl)(methyl)amino]ethyl}-N-methylcarbamate as a yellow oil (62%; at 80% purity). ¹H NMR (500 MHz, CDCl₃) δ: 7.61-7.36 (m, 1H), 6.14-5.72 (m, 1H), 5.43-5.23 (m, 1H), 4.08-3.92 (m, 1H), 3.76-3.57 (m, 1H), 3.51-3.19 (m, 5H), 2.99-2.62 (m, 6H), 2.60-2.30 (m, 3H), 2.28-2.14 (m, 3H), 2.13-1.94 (m, 4H), 1.76-1.51 (m, 6H), 1.51-1.37 (m, 9H), 1.00-0.79 (m, 18H), 0.13-0.03 (m, 6H).

### Step-7: Synthesis of tert-butyl N-{2-[({3-[(3R)-3-[(tert-butyldimethylsilyl)oxy]-4,4-dimethylcyclohexyl]-1-(oxan-2-yl)-1H-pyrazol-4-yl}methyl)(methyl)amino]ethyl}-N-methylcarbamate

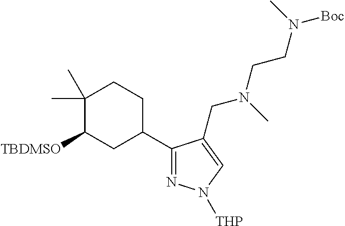

A solution of tert-butyl N-{2-[({3-[(3R)-3-[(tert-butyldimethylsilyl)oxy]-4,4-dimethylcyclohex-1-en-1-yl]-1-(oxan-2-yl)-1H-pyrazol-4-yl}methyl)(methyl) amino]ethyl}-N-methylcarbamate (80%, 1 g, 1.35 mmol) in EtOH (10 ml) was cautiously added onto a purged [N2] suspension of Raney-Nickel catalyst (2.5 ml) in EtOH (20 ml). The resulting solution was purged with Nitrogen (3×), Hydrogen (2×) and left under an atmosphere of Hydrogen at rt for 16 h. An additional 7.5 ml of catalyst were added and the reaction was left stirring under hydrogen atmosphere for 6 h, after which time LCMS showed complete conversion to the desired TM. The solution was filtered on celite and the pad was washed with EtOAc (150 ml). The filtrate was evaporated under reduced pressure and co-evaporated with toluene to afford 870 mg (92%; at 85% purity) of tert-butyl N-{2-[({3-[(3R)-3-[(tert-butyldimethylsilyl)oxy]-4,4-dimethylcyclohexyl]-1-(oxan-2-yl)-1H-pyrazol-4-yl}methyl)(methyl)amino]ethyl}-N-methylcarbamate as light yellow oil. LC/MS 1.62 min; m/z 593.35 [M+1]. ¹H NMR (250 MHz, CDCl₃) δ: 7.45 (d, J=11.3 Hz, 1H), 5.41-5.17 (m, 1H), 4.06 (d, J=8.3 Hz, 1H), 3.67 (t, J=11.2 Hz, 1H), 3.34 (d, J=8.8 Hz, 6H), 2.85 (d, J=12.0 Hz, 5H), 2.68 (s, 1H), 2.47 (s, 2H), 2.20 (d, J=9.2 Hz, 3H), 2.02 (s, 3H), 1.93-1.36 (m, 13H), 1.25 (d, J=6.2 Hz, 2H), 1.06-0.79 (m, 15H), 0.02 (d, J=7.0 Hz, 6H).

### Step-8: Synthesis of tert-butyl N-{2-[({3-[(3R)-3-hydroxy-4,4-dimethylcyclohexyl]-1-(oxan-2-yl)-1H-pyrazol-4-yl}methyl)(methyl)amino]ethyl}-N-methylcarbamate

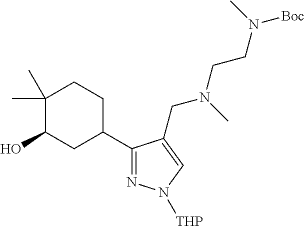

Tert-butyl N-{2-[({3-[(3R)-3-[(tert-butyldimethylsilyl)oxy]-4,4-dimethylcyclohexyl]-1-(oxan-2-yl)-1H-pyrazol-4-yl}methyl)(methyl)amino]ethyl}-N-methylcarbamate (85%, 870 mg, 1.25 mmol) was dissolved in 1M TBAF in THF (12 ml). The reaction was heated to 60° C. and stirred for 16 h. The reaction was quenched with water (10 ml) and was extracted with EtOAc (3×20 ml). The combined organic layers were dried over Na₂SO₄ and evaporated to dryness. The residue was purified by Biotage (SNAP 50 g, eluent Hep/EtOAc 95/5 to 60/40) to afford 450 mg of tert-butyl N-{2-[({3-[(3R)-3-hydroxy-4,4-dimethylcyclohexyl]-1-(oxan-2-yl)-1H-pyrazol-4-yl}methyl)(methyl)amino]ethyl}-N-methylcarbamate as a light yellow oil (60%; at 80% purity); NMR showed presence of TBAF residue. Material used as is in the next step. ¹H NMR (250 MHz, CDCl₃) δ: 7.53-7.31 (m, 1H), 5.30-5.13 (m, 1H), 3.98 (d, J=10.3 Hz, 1H), 3.60 (td, J=11.1, 2.8 Hz, 1H), 3.31 (dd, J=12.4, 6.8 Hz, 9H), 2.89 (dd, J=10.3, 6.5 Hz, 1H), 2.78 (d, J=13.1 Hz, 9H), 2.50-2.29 (m, 2H), 2.12 (s, 3H), 1.91 (d, J=20.2 Hz, 4H), 1.77-1.47 (m, 8H), 1.37 (d, J=7.5 Hz, 26H), 1.24-1.12 (m, 2H), 1.04-0.83 (m, 8H).

### Step-9: Synthesis of tert-butyl N-{2-[({3-[(3R)-4,4-dimethyl-3-(2-methylpropoxy)cyclohexyl]-1-(oxan-2-yl)-1H-pyrazol-4-yl}methyl)(methyl)amino]ethyl}-N-methyl carbamate

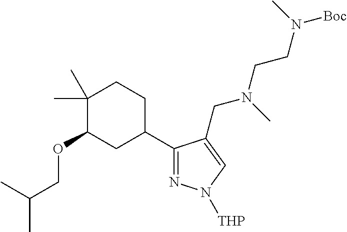

0.91M KHMDS (7.46 ml) and 18-C-6 (40 mg, 0.15 mmol) were added to a solution of tert-butyl N-{2-[({3-[(3R)-3-hydroxy-4,4-dimethylcyclohexyl]-1-(oxan-2-yl)-1H-pyrazol-4-yl}methyl)(methyl)amino] ethyl}-N-methylcarbamate (650 mg, 1.36 mmol) in dry toluene (20 ml). The reaction was stirred at er for 1 h, and then 1-bromo-2-methylpropane (443 μl, 4.07 mmol) was added. The reaction was stirred at rt for 2 d; no reaction was detected. The reaction was heated to 60° C. with further lots of 0.91M KHMDS (7.46 ml) and 1-bromo-2-methylpropane (443 μl, 4.07 mmol) added daily over 8 d. The solution was washed with water (25 ml) and extracted with EtOAc (3×30 ml). The combined organic layers were dried over Na₂SO₄ and evaporated to dryness. The crude residue was purified by low pH prep HPLC in four injections to afford 15 mg of the desired tert-butyl N-{2-[({3-[(3R)-4,4-dimethyl-3-(2-methylpropoxy)cyclohexyl]-1-(oxan-2-yl)-1H-pyrazol-4-yl}methyl)(methyl)amino]ethyl}-N-methyl carbamate (2%). 220 mg of starting material were also recovered. LC/MS 1.41 min; m/z=535.30 [M+1]. ¹H NMR (500 MHz, CDCl₃) δ: 7.66-7.40 (m, 1H), 5.27 (s, 1H), 4.05 (d, J=9.8 Hz, 1H), 3.74-3.59 (m, 1H), 3.57-3.27 (m, 5H), 3.05-2.96 (m, 1H), 2.91 (dd, J=11.5, 4.0 Hz, 1H), 2.84 (s, 3H), 2.73-2.57 (m, 2H), 2.57-2.44 (m, 1H), 2.34-2.20 (m, 3H), 2.11-1.96 (m, 4H), 1.81-1.52 (m, 7H), 1.44 (s, 10H), 1.34-1.23 (m, 1H), 1.05-0.97 (m, 3H), 0.97-0.91 (m, 4H), 0.91-0.85 (m, 5H).

### Step-10: Synthesis of ({3-[(3R)-4,4-dimethyl-3-(2-methylpropoxy)cyclohexyl]-1H-pyrazol-4-yl}methyl)(methyl)[2-(methylamino)ethyl]amine trihydrochloride

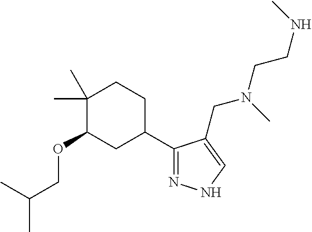 [SMILES: CNCCN(C)Cc1c[nH]nc1C1CCC(C)(C)[C@H](OCC(C)C)C1]

Tert-butyl N-{2-[({3-[(3R)-4,4-dimethyl-3-(2-methylpropoxy)cyclohexyl]-1-(oxan-2-yl)-1H-pyrazol-4-yl}methyl)(methyl)amino]ethyl}-N-methylcarbamate (15 mg, 0.03 mmol) was dissolved in dioxane (2 ml), HCl (6N, 2 ml) was added and the reaction was stirred at rt for 1 h. The solvents were removed under reduced pressure to afford the 13 mg of ({3-[(3R)-4,4-dimethyl-3-(2-methylpropoxy)cyclohexyl]-1H-pyrazol-4-yl}methyl)(methyl)[2-(methylamino)ethyl]amine trihydrochloride as an off white solid (100%). LC/MS: 2.66 min, m/z=351.10 [M+1]. ¹H NMR (500 MHz, Methanol-d4) δ: 8.40 (s, 1H), 4.52 (s, 2H), 3.86-3.53 (m, 4H), 3.42 (s, 1H), 3.28 (m, 2H), 2.93 (s, 3H), 2.80 (s, 3H), 2.23-2.04 (m, 1H), 1.97-1.49 (m, 6H), 1.29 (s, 1H), 1.05 (s, 3H), 1.00 (s, 3H), 0.91 (dd, J=11.9, 6.5 Hz, 6H).

### Example B-2. Synthesis of {2-[({3-[(3R)-3-[(3-methoxypropoxy)methyl]-4,4-dimethylcyclohexyl]-1H-pyrazol-4-yl}methyl)(methyl)amino]ethyl}(methyl)amine (Compound 30)

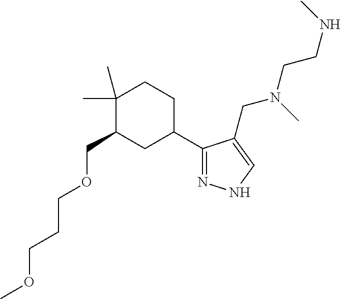 [SMILES: CNCCN(C)Cc1c[nH]nc1C1CCC(C)(C)[C@H](COCCCOC)C1]

### Step-1: Synthesis of (2R)-2-(methoxymethyl)-N-methylidenepyrrolidin-1-amine

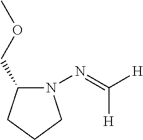

To a suspension of paraformaldehyde (4.5 g, 150 mmol) in DCM (450 ml) at 0° C. was added dropwise a solution of (2R)-2-(methoxymethyl)pyrrolidin-1-amine (15 g, 115 mmol) in DCM (150 ml). The resulting mixture was stirred at rt for 2 d. The reaction mixture was dried over Na₂SO₄, filtered and concentrated. The desired product was purified by distillation under reduced pressure at 44° C. under ˜0.1 mbar to yield 15.56 g of imine as a colorless oil (95%). ¹H NMR (500 MHz, CDCl₃) δ: 6.12 (d, J=11.6 Hz, 1H), 6.02 (d, J=11.7 Hz, 1H), 3.58-3.53 (m, 2H), 3.46-3.41 (m, 1H), 3.37 (s, 3H), 3.32 (ddd, J=9.9, 7.2, 3.3 Hz, 1H), 2.83 (q, J=8.0 Hz, 1H), 2.04-1.77 (m, 4H).

### Step-2: Synthesis of (3R)-3-[(E)-N-[(2R)-2-(methoxymethyl)pyrrolidin-1-yl]carboximidoyl]-4,4-dimethylcyclohexan-1-one

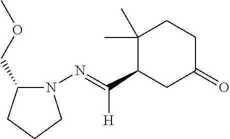 [SMILES: COC[C@H]1CCCN1/N=C/[C@@H]1CC(=O)CCC1(C)C]

To a cooled (−78° C.) solution of 4,4-dimethylcyclohex-2-en-1-one (16.99 g, 137 mmol) in dry THF (300 mL) were sequentially added TBDMSOTf (27.67 ml, 120 mmol) and a pre-cooled (−78° C.) solution of imine (15.56 g, 109 mmol) in dry THF (150 ml) under N₂ atmosphere. After 3 h at −78° C., 1M TBAF in THF (164 ml) was added and the mixture was allowed to warm to rt and stirred until LC/MS indicated total consumption of the silyl enol ether (2 d). LCMS analysis product formation (52%, 1.08 min, m/z=267.10 [M+1]. TBME (800 ml) was added and the mixture was washed with water (3×200 ml). The combined aqueous layers were extracted with TBME (200 ml). The combined organic layers were washed with brine (200 ml), dried over Na₂SO₄, filtered and concentrated. The brown residue was purified by Isolera over SiO₂ (340 g), dry loaded and eluted with a gradient of EtOAc in Hept from 0 to 100% to yield 24.92 g of desired product as a yellowish oil (85%). LCMS: 1.1 min, m/z=267.1 [M+1]. ¹H NMR (250 MHz, CDCl₃) δ: 6.55 (d, J=4.4 Hz, 1H), 3.61-3.26 (m, 7H), 2.73 (q, J=8.4 Hz, 1H), 2.58-2.22 (m, 5H), 2.02-1.52 (m, 6H), 1.07 (d, J=5.5 Hz, 6H).

### Step-3: Synthesis of (1R)-2,2-dimethyl-5-oxocyclohexane-1-carbaldehyde

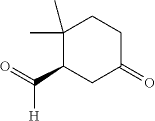

2 Batches of ˜7.4 g: In a 2 neck RBF (1 L), a solution of hydrazone (7.46 g, 28 mmol) in dry DCM (300 ml) was bubbled with nitrogen at −78° C. for 10 min, then nitrogen was replaced by ozone until appearance of a permanent green/blue colour (3-4 h). Ozone was replaced with Nitrogen for 10 min then DMS (4.14 ml, 56 mmol) was added. The mixture was transferred into a RBF and solvent evaporated. The residue was purified by Isolera over SiO₂ (340 g), dry loaded and eluted with a gradient of EtOAc in Hept from 0 to 50%. Both batches of product isolated after purification were combined to yield 3.52 g of aldehyde as a yellow oil (40.8%). ¹H NMR (500 MHz, CDCl₃) δ: 9.85 (d, J=1.5 Hz, 1H), 2.68-2.54 (m, 2H), 2.50-2.27 (m, 3H), 1.80-1.70 (m, 2H), 1.32 (s, 3H), 1.15 (s, 3H).

### Step-4: Synthesis of (1R)-2,2-dimethyl-5-oxocyclohexane-1-carboxylic acid

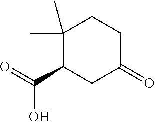

To a solution of aldehyde (2.92 ml, 18.6 mmol) in ether (150 ml) at −30° C. was added dropwise 2M Jones' reagent (27.8 ml). After 30 min at −30° C. the mixture was allowed to reach rt under stirring for another ˜2 hr. The reaction was cooled to 0° C. and basified with 1N NaOH (˜300 ml). The aqueous layer was washed with TBME (2×400 ml), then acidified with 2M H₂SO₄ (100 ml). The product was extracted with EtOAc (5×250 ml). The combined organic layers were dried over Na₂SO₄, filtered and concentrated. The residue was co-evaporated with heptane (˜100 ml) and the residue was purified by Isolera over SiO₂ (50 g), eluted with a gradient of MeOH in DCM from 0 to 10% to yield 2.37 g of the acid as a light yellow crystalline solid (75.1%). LCMS 0.83 min, m/z=171.05 [M+1]. ¹H NMR (250 MHz, CDCl₃) δ: 2.73-2.26 (m, 5H), 1.90 (dt, J=12.7, 6.2 Hz, 1H), 1.66 (ddd, J=14.2, 9.2, 5.8 Hz, 1H), 1.22 (s, 3H), 1.17 (s, 3H).

### Step-5: Synthesis of methyl (1R)-2,2-dimethyl-5-oxocyclohexane-1-carboxylate

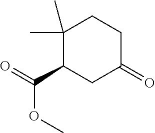

A mixture of acid (2.37 g, 14 mmol), K₂CO₃ (2.12 g, 15 mmol) and MeI (0.95 ml, 15 mmol) in acetone was sealed and heated to 60° C. for ˜6 h. The reaction was cooled to rt, poured onto water (75 ml) and the desired ester was extracted with EtOAc (150 ml). The organic was washed with water (2×25 ml), brine (25 ml), dried over Na₂SO₄, filtered and concentrated. The residue was purified by Isolera over SiO₂ (100 g), eluted with a gradient of EtOAc in Hept from 0 to 60% to yield 2.06 g of the methyl ester as a colourless oil (80%). LCMS 1.03 min, m/z=185 [M+1]. ¹H NMR (500 MHz, CDCl₃) δ: 3.69 (s, 3H), 2.68-2.57 (m, 2H), 2.49-2.39 (m, 2H), 2.37-2.29 (m, 1H), 1.91-1.85 (m, 1H), 1.69-1.61 (m, 1H), 1.16 (s, 3H), 1.12 (s, 3H).

### Step-6: Synthesis of methyl (1R)-6,6-dimethyl-3-(trifluoromethanesulfonyloxy) cyclohex-3-ene-1-carboxylate

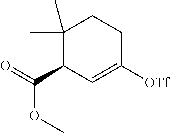 [SMILES: COC(=O)[C@@H]1C=C(OS(=O)(=O)C(F)(F)F)CCC1(C)C]

To a cold (0° C.) solution of methyl 2,2-dimethyl-5-oxocyclohexanecarboxylate (2.06 g, 11.2 mmol) in DCE (50 ml) was slowly added 2,6-di-tert-butylpyridine (2.76 ml, 12.3 mmol) followed by Tf₂O (2.07 ml, 12.3 mmol). The reaction was allowed to reach rt for 16 h. The solvent was evaporated and the residue was dissolved in EtOAc (150 ml), washed with sat sol of NaHCO₃ (50 ml), brine (50 ml) and dried over Na₂SO₄. The residue was purified by Isolera over SiO₂ (100 g) eluted with a gradient of EtOAc in Hept from 0 to 20% to yield 2.67 g of enol triflate as a clear yellow oil (75%). LCMS 1.47 min, m/z=316.8 [M+1]. ¹H NMR (500 MHz, CDCl₃) δ: 5.71 (t, J=3.9 Hz, 1H), 3.69 (s, 3H), 2.66 (ddq, J=16.9, 7.8, 2.8 Hz, 1H), 2.56 (dd, J=8.9, 5.6 Hz, 1H), 2.49-2.41 (m, 1H), 2.08 (dt, J=4.2, 2.6 Hz, 2H), 1.04 (s, 3H), 1.01 (s, 3H).

### Step-7: Synthesis of methyl (1R)-6,6-dimethyl-3-(tetramethyl-1,3,2-dioxaborolan-2-yl)cyclohex-3-ene-1-carboxylate

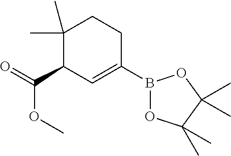 [SMILES: COC(=O)[C@@H]1C=C(B2OC(C)(C)C(C)(C)O2)CCC1(C)C]

A solution of enol trifoliate (2.67 g, 8.44 mmol), potassium acetate (6.21 g, 63.31 mmol) and bis(pinacolato)diboron (2.36 g, 9.29 mmol) in dioxane (90 ml) was bubbled with N₂ for 10 min. A solution of PdCl₂(dppf)-DCM (0.34 g, 0.42 mmol) in dioxane (10 ml) was added and the reaction mixture was bubbled with N₂ for a further 5 min. The reaction mixture was heated to 80° C. under N₂ for 3 h then at rt for 16 h. Water (100 ml) was added and the product was extracted with EtOAc (100 and 50 ml). The combined organic layers were washed with water (2×50 ml), brine (50 ml), dried over Na₂SO₄, filtered and concentrated. The residue was purified by Isolera over SiO₂ (100 g), dry loaded and eluted with a gradient of EtOAc in Hept from 0 to 10% to yield 1.96 g of enol boronate as a white solid (79%). LCMS 1.56 min, m/z=295 [M+1]. ¹H NMR (500 MHz, CDCl₃) δ: 6.53-6.35 (m, 1H), 3.63 (s, 3H), 2.42-2.28 (m, 3H), 2.03-1.89 (m, 2H), 1.25 (s, 12H), 0.96 (d, J=8.5 Hz, 6H).

### Step-8: Synthesis of methyl (1R)-3-(4-{[(2-{[(tert-butoxy)carbonyl](methyl)amino}ethyl)(methyl)amino] methyl}-1-(oxan-2-yl)-1H-pyrazol-3-yl)-6,6-dimethylcyclohex-3-ene-1-carboxylate

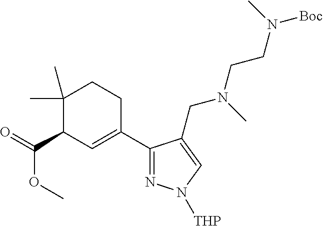

A solution of enol boronate (1.96 g, 6.66 mmol), iodide (3.19 g, 6.66 mmol) and K₂CO₃ (2.76 g, 20 mmol) in dioxane (80 ml) and water (15 ml) was degassed for 15 min then a solution of PdCl₂(dppf)-DCM (0.54 g, 0.67 mmol) in dioxane (10 ml) was added and degassed for another 5 min. ˜ The resulting solution was heated to 80° C. under N₂ for 16 h. LCMS analysis shows reaction complete. The reaction was cooled to rt and the solvent was evaporated. The residue was purified over by Isolera over SiO₂ (100 g), dry loaded and eluted with a gradient of THF in heptane from 17 to 100% to yield 3.53 g of product as a viscous amber oil (97%). LCMS 1.1 min, m/z=519.3 [M+1]. ¹H NMR (500 MHz, CDCl₃) δ: 7.46 (s, 1H), 6.11 (s, 1H), 5.28 (dt, J=9.8, 2.8 Hz, 1H), 4.10-3.99 (m, 1H), 3.66 (s, 3H), 3.34 (dd, J=34.7, 21.2 Hz, 4H), 2.82 (s, 3H), 2.58-2.40 (m, 3H), 2.21 (s, 3H), 2.15-1.92 (m, 6H), 1.70-1.55 (m, 5H), 1.43 (s, 9H), 1.03 (d, J=1.8 Hz, 6H).

### Step-9: Synthesis of methyl (1R)-5-(4-{[(2-{[(tert-butoxy)carbonyl](methyl)amino}ethyl)(methyl)amino]methyl}-1-(oxan-2-yl)-1H-pyrazol-3-yl)-2,2-dimethylcyclohexane-1-carboxylate

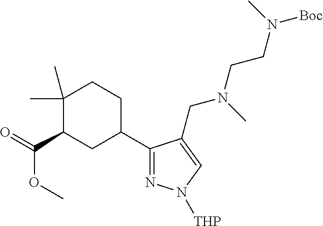

A solution of methyl (1R)-3-(4-{[(2-{[(tert-butoxy)carbonyl](methyl)amino}ethyl)(methyl)amino] methyl}-1-(oxan-2-yl)-1H-pyrazol-3-yl)-6,6-dimethylcyclohex-3-ene-1-carboxylate (95%, 3.53 g, 6.47 mmol) in EtOH (30 ml) was cautiously added onto a suspension of Raney Ni (11.47 ml, 129 mmol) in EtOH (20 ml). The resulting solution was purged with N₂ (3×), H₂ (2×) and left under an atmosphere of H₂ at rt for 16 h. LCMS analysis next morning showed reduced iodide impurity but not reduction of the double bond. Celite (˜25 g) was added and mixture filtered through more Celite (˜25 g). The solid was washed with EtOAc-MeOH (2×50 ml, 1:1) and the solvent was evaporated. The starting material was recovered by SiO₂ Isolera purification and was resubmitted to same conditions. LCMS analysis next morning showed complete reaction. Celite (10 g) was added and mixture stirred for 10 min. The mixture was filtered through a pad of Celite (˜10 g), and the residue was washed with EtOAc-MeOH (1:1, 2×50 ml). The combined organic layers were concentrated and the residue was purified by Isolera over SiO₂ (50 g), dry loaded and eluted with a gradient of THF in Hept from ˜10 to 100% to yield 2.89 g (86%) of product as a colourless viscous oil as a ˜5:1 mixture of cis-trans isomers. LCMS 1.11+1.23 min, m/z=521.3 [M+1]. ˜5:1 mixture of cis-trans isomers. ¹H NMR (500 MHz, CDCl₃) δ: 7.41 (s, 1H), 5.33-5.22 (m, 1H), 4.09-4.01 (m, 1H), 3.63 (s, 4H), 3.31 (d, J=31.5 Hz, 4H), 2.82 (s, 3H), 2.66 (tt, J=12.5, 3.7 Hz, 1H), 2.51 (d, J=49.7 Hz, 2H), 2.35-2.30 (m, 1H), 2.20 (s, 3H), 2.13-1.97 (m, 4H), 1.44 (s, 9H), 1.03 (d, J=7.3 Hz, 6H).

### Step-10: Synthesis of tert-butyl N-{2-[({3-[(3R)-3-(hydroxymethyl)-4,4-dimethylcyclohexyl]-1-(oxan-2-yl)-1H-pyrazol-4-yl}methyl)(methyl)amino]ethyl}-N-methylcarbamate

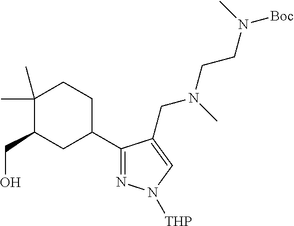

To a solution of methyl (1R)-5-(4-{[(2-{[(tert-butoxy)carbonyl](methyl)amino}ethyl)(methyl)amino]methyl}-1-(oxan-2-yl)-1H-pyrazol-3-yl)-2,2-dimethylcyclohexane-1-carboxylate (200 mg, 0.38 mmol) in dry THF (5 ml) at −78° C. was added 1M LiAlH₄ (384 μl). The reaction monitored at −78° C. LCMS analysis after ˜2 h at −78° C. shows mainly SM untouched. Another 1M LiAlH₄ (384 μl) was added and reaction allowed to reach rt. LCMS analysis after 1 h at rt shows mainly product. The reaction was cooled to 0° C. and ˜30 l of water was carefully added followed by 30 l of 15% NaOH, 90 l of water. After 10 min, EtOAc was added (25 ml) with ˜250 mg of Na₂SO₄. The mixture was stirred for 5 min, filtered through a pad of Celite (˜10 g) and the solid residue was washed with EtOAc (2×10 ml) and concentrated. The residue was purified by Isolera over SiO₂ (25 g), eluted with a gradient of MeOH in EtOAc from 3 to 30% to yield 122 mg (65%) of alcohol as a colourless viscous oil. LCMS 1.13 min, m/z=493.3 [M+1]. ¹H NMR (250 MHz, CDCl₃) δ: 7.40 (s, 1H), 5.36-5.19 (m, 1H), 4.08-3.98 (m, 1H), 3.89-3.77 (m, 1H), 3.76-3.58 (m, 2H), 3.49-3.21 (m, 5H), 2.81 (s, 3H), 2.76-2.35 (m, 3H), 2.20 (s, 3H), 2.01 (d, J=8.4 Hz, 5H), 1.82-1.51 (m, 6H), 1.41 (s, 11H), 1.00 (s, 3H), 0.85 (s, 3H).

### Step-11: Synthesis of tert-butyl N-{2-[({3-[(3R)-3-[(3-methoxypropoxy)methyl]-4,4-dimethylcyclohexyl]-1-(oxan-2-yl)-1H-pyrazol-4-yl}methyl)(methyl)amino]ethyl}-N-methylcarbamate

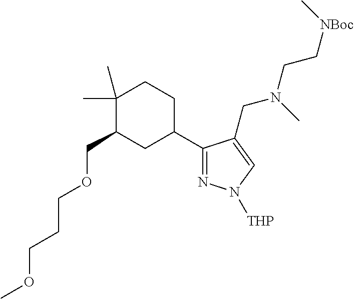

In a sealed tube under N₂ was placed NaH 60% in oil (60%, 40 mg, 0.99 mmol) in dry DMF (1 ml). The suspension was cooled to 0° C. and a solution of tert-butyl N-{2-[({3-[(3R)-3-(hydroxymethyl)-4,4-dimethylcyclohexyl]-1-(oxan-2-yl)-1H-pyrazol-4-yl}methyl)(methyl)amino]ethyl}-N-methylcarbamate (122 mg, 0.25 mmol) in DMF (4 ml) was added and the reaction allowed to rt. After 15 min, 1-bromo-3-methoxypropane (56 μl, 0.5 mmol) was added and reaction monitored by LCMS at rt. LCMS analysis after ˜3 h at rt shows mainly SM and no sign of desired product. Fresh NaH and bromide were added and temperature was raised to 60° C. for 16 h. LCMS analysis next morning shows product formation (weak UV response, ˜29%, 1.33 min, m/z=565.35 [M+1] and some remaining SM. Fresh NaH and bromide were added and temperature was raised to 60° C. 16 h. LCMS analysis shows no change. The reaction was cooled to rt and poured onto water (25 ml) and EtOAc (25 ml). The aqueous layer was separated and extracted with EtOAc (25 ml). The combined organic layers were washed with water (3×15 ml), brine (15 ml), dried over Na₂SO₄, filtered and concentrated. The residue was purified by Isolera over SiO₂ (10 g) eluted with a gradient of MeOH in EtOAc from 0 to 20% to yield 30 mg (22%) of the ether as a colourless viscous oil. LCMS 1.33 min, m/z=565.35 [M+1]. ¹H NMR (500 MHz, CDCl₃) δ: 7.40 (s, 1H), 5.27 (dd, J=8.3, 3.7 Hz, 1H), 4.06-4.00 (m, 1H), 3.65 (td, J=14.5, 12.8, 3.8 Hz, 1H), 3.55 (dd, J=6.1, 2.2 Hz, 1H), 3.47-3.23 (m, 12H), 3.10-3.03 (m, 1H), 2.80 (d, J=4.1 Hz, 3H), 2.63 (td, J=11.4, 3.7 Hz, 1H), 2.45 (s, 2H), 2.17 (d, J=20.0 Hz, 8H), 1.89-1.48 (m, 9H), 1.42 (s, 9H), 0.98 (s, 3H), 0.84 (s, 3H).

### Step-12: Synthesis of {2-[({3-[(3R)-3-[(3-methoxypropoxy)methyl]-4,4-dimethylcyclohexyl]-1H-pyrazol-4-yl}methyl)(methyl)amino]ethyl}(methyl)amine

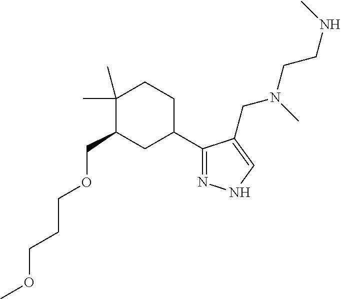 [SMILES: CNCCN(C)Cc1c[nH]nc1C1CCC(C)(C)[C@H](COCCCOC)C1]

A solution of tert-butyl N-{2-[({3-[(3R)-3-[(3-methoxypropoxy)methyl]-4,4-dimethylcyclohexyl]-1-(oxan-2-yl)-1H-pyrazol-4-yl}methyl)(methyl)amino]ethyl}-N-methylcarbamate (30 mg, 0.05 mmol) in dioxane (3 ml) was treated with 6M HCl in water (0.5 ml) at rt 16 h. LCMS analysis next morning shows no UV response but product mainly observed in LCMS: (0.93 min, m/z=381.2, [M+1]). The solvent was evaporated to dryness and residue was purified over SCX-2 (1 g) to yield product contaminated by cleaved ether (from previous step). The material was purified by Isolera over SiO₂ (10 g), eluted with a gradient of 7N NH₃ in MeOH in DCM from 0 to 20% to 11.1 mg (55%) of final product as a yellow viscous oil. LCMS: 5.09 min, m/z=381.4 [M+1]. ¹H NMR (500 MHz, Methanol-d4) δ: 7.43 (s, 1H), 3.68-3.39 (m, 7H), 3.29 (s, 3H), 3.13 (t, J=8.8 Hz, 1H), 2.75 (t, J=6.4 Hz, 3H), 2.54 (t, J=6.2 Hz, 2H), 2.41 (s, 3H), 2.20 (s, 3H), 1.96-1.88 (m, 1H), 1.87-1.38 (m, 8H), 1.04 (s, 3H), 0.93 (s, 3H).

### Example B-3. Synthesis of (1S)—N,2,2-trimethyl-5-[4-({methyl[2-(methylamino)ethyl]amino}methyl)-1H-pyrazol-3-yl]-N-(2-methylpropyl)cyclohexane-1-carboxamide (Compound 25)

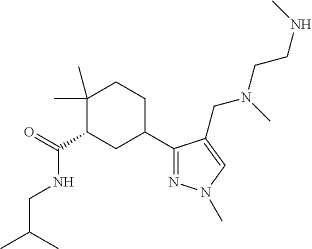 [SMILES: CNCCN(C)Cc1cn(C)nc1C1CCC(C)(C)[C@@H](C(=O)NCC(C)C)C1]

### Step-1: Synthesis of tert-butyl 4-{[(2-{[(tert-butoxy)carbonyl](methyl)amino}ethyl)(methyl)amino] methyl}-3-[(3S)-4,4-dimethyl-3-[(2-methylpropyl)carbamoyl]cyclohexyl]-1H-pyrazole-1-carboxylate

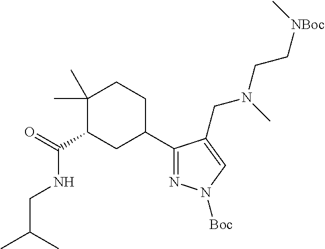 [SMILES: CC(C)CNC(=O)[C@H]1CC(c2nn(C(=O)OC(C)(C)C)cc2CN(C)CCN(C)C(=O)OC(C)(C)C)CCC1(C)C]

A solution of (1S)-2,2-dimethyl-5-[4-({methyl[2-(methylamino)ethyl]amino}methyl)-1H-pyrazol-3-yl]-N-(2-methylpropyl)cyclohexane-1-carboxamide (90 mg, 0.24 mmol), Et₃N (100 μl, 0.72 mmol), di-tert-butyl dicarbonate (156 mg, 0.72 mmol) and catalytic amount of N,N-dimethylpyridin-4-amine (1.46 mg, 0.01 mmol) in MeCN (10 ml) was stirred at rt for 2 d. LCMS analysis after 2 days shows no UV but product formation mainly observed in MS chromatogram. The reaction was poured into water (25 ml), and product was extracted with EtOAc (2×35 ml). The combined organic were washed with water (10 ml), brine (10 ml), dried over Na₂SO₄, filtered and concentrated. The residue was purified by Isolera over SiO₂ (10 g), eluted with a gradient of THF in Hept from 15 to 100% to yield 111 mg (81%) of bis boc protected product as an orange viscous oil. LCMS: 1.2 min, m/z=578.35 [M+1].

### Step-2: Synthesis of tert-butyl 4-{[(2-{[(tert-butoxy)carbonyl](methyl)amino}ethyl)(methyl)amino] methyl}-3-[(3S)-4,4-dimethyl-3-[methyl(2-methylpropyl)carbamoyl]cyclohexyl]-1H-pyrazole-1-carboxylate

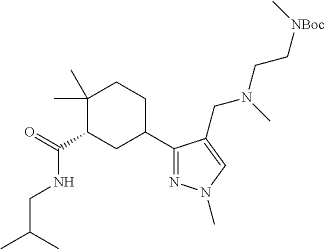 [SMILES: CC(C)CNC(=O)[C@H]1CC(c2nn(C)cc2CN(C)CCN(C)C(=O)OC(C)(C)C)CCC1(C)C]

A solution of amide (111 mg, 0.19 mmol) in THF (5 ml) was added onto a suspension of NaH 60% in oil (60%, 16 mg, 0.41 mmol) in THF (5 ml) at 0° C. After ˜5 min, neat MeI (18 μl, 0.28 mmol) was added and reaction stirred at rt 16 h. LCMS analysis shows product formation (100%, 1.10 min, [MH]⁺=492.35. The reaction was poured onto sat sol of NH₄Cl (20 ml), water (20 ml) and product was extracted with EtOAc (50 ml). The organic layer was washed with water (20 ml), brine (20 ml), dried over Na₂SO₄, filtered and concentrated. The residue was purified by Isolera over SiO₂ (10 g), eluted with a gradient of MeOH in DCM from 0 to 10% to yield methylated pyrazole by-product only as a yellow viscous oil. LCMS 1.11 min, m/z=492.3 [M+1].

### Step-3: Synthesis of (1S)—N,2,2-trimethyl-5-[4-({methyl[2-(methylamino)ethyl]amino}methyl)-1H-pyrazol-3-yl]-N-(2-methylpropyl)cyclohexane-1-carboxamide

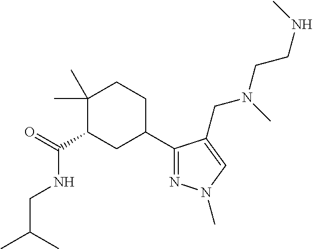 [SMILES: CNCCN(C)Cc1cn(C)nc1C1CCC(C)(C)[C@@H](C(=O)NCC(C)C)C1]

A solution of tert-butyl 4-{[(2-{[(tert-butoxy)carbonyl](methyl)amino}ethyl)(methyl)amino] methyl}-3-[(3S)-4,4-dimethyl-3-[methyl(2-methylpropyl)carbamoyl]cyclohexyl]-1H-pyrazole-1-carboxylate (36 mg, 0.07 mmol) in dioxane (2 ml) was treated with 6M HCl in Water (1.5 ml) at rt. LCMS analysis after ˜3 h shows reaction complete. The solvent was evaporated and the residue was purified over SCX-2 (1 g), eluted with MeOH-DCM (1:1, 4×2 ml) then 7N NH₃ in MeOH-DCM (1:1, 4×2 ml). Evaporation of the appropriate fractions afforded desired product as a colorless oil. LCMS analysis (METCR1673 Generic 2 minutes), No UV response, 0.91 min, m/z=392.15 [M+1]. ¹H NMR (500 MHz, CDCl₃) δ: 7.22 (s, 1H), 5.67 (m, 1H), 3.83 (2s, 3H), 3.35 (dd, J=19.2, 3.8 Hz, 2H), 3.18-2.98 (m, 2H), 2.73-2.63 (m, 3H), 2.50 (td, J=5.9, 2.6 Hz, 2H), 2.43 (2s, 3H), 2.14 (2s, 3H), 2.10-1.94 (m, 2H), 1.93-1.51 (m, 6H), 1.37 (td, J=13.5, 3.7 Hz, 1H), 1.12 (2s, 3H), 1.06 (2s, 3H), 0.91 (2d, J=6.6 Hz, 6H). Correlation seen between H2-C28 on the HMBC and NOE between H2-H28 suggest the presence of the methyl on the pyrazole ring.

### Example B-4. Synthesis of {2-[({3-[(3R)-3-(ethoxymethyl)-4,4-dimethylcyclohexyl]-1H-pyrazol-4-yl}methyl)(methyl) amino]ethyl}(methyl)amine (Compound 31)

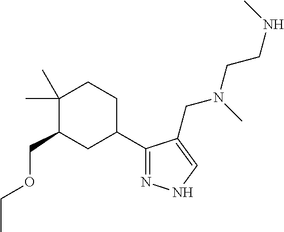 [SMILES: CCOC[C@@H]1CC(c2n[nH]cc2CN(C)CCNC)CCC1(C)C]

### Step-1: Synthesis of tert-butyl N-{2-[({3-[(3R)-3-(ethoxymethyl)-4,4-dimethylcyclohexyl]-1-(oxan-2-yl)-1H-pyrazol-4-yl}methyl)(methyl)amino]ethyl}-N-methylcarbamate

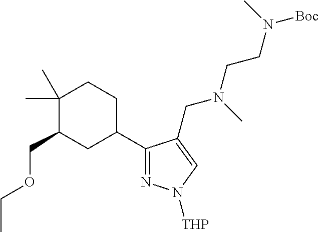

At rt, sodium hydride (60%, 100 mg, 2.54 mmol) was added to a solution of tert-butyl N-{2-[({3-[(3R)-3-(hydroxymethyl)-4,4-dimethylcyclohexyl]-1-(oxan-2-yl)-1H-pyrazol-4-yl}methyl)(methyl)amino]ethyl}-N-methylcarbamate (0.25 g, 0.51 mmol) in THF (10 ml). The solution was stirred 30 min before adding iodoethane (81 μl, 1.01 mmol). The reaction was stirred at 70° C. 16 h. EtOAc (20 ml) was added and the solution was washed with brine (20 ml). The aqueous was extracted with EtOAc (2×20 ml). The combined organic layers were dried over Na₂SO₄ and evaporated to dryness. The residue was purified by Biotage over SiO₂ (SNAP 50 g, eluent EtOAc/MeOH 95/5 to 70/30) to afford 140 mg of tert-butyl N-{2-[({3-[(3R)-3-(ethoxymethyl)-4,4-dimethylcyclohexyl]-1-(oxan-2-yl)-1H-pyrazol-4-yl}methyl)(methyl)amino]ethyl}-N-methylcarbamate (49%) as a light yellow oil and 40 mg of a lower purity fraction (14%) as a light yellow oil. LC/MS 1.17 min; m/z 521.90 [M+1]; ¹H NMR (500 MHz, CDCl₃) δ: 7.42 (s, 1H), 5.35-5.25 (m, 1H), 4.08-4.01 (m, 1H), 3.67 (td, J=11.3, 2.3 Hz, 1H), 3.63-3.52 (m, 1H), 3.50-3.22 (m, 6H), 3.06 (td, J=8.7, 3.5 Hz, 1H), 2.83 (s, 3H), 2.72-2.59 (m, 1H), 2.55-2.40 (m, 2H), 2.26-2.15 (m, 3H), 2.04-1.94 (m, 4H), 1.78-1.59 (m, 5H), 1.58-1.48 (m, 3H), 1.48-1.41 (m, 9H), 1.37 (dd, J=13.1, 4.3 Hz, 1H), 1.21-1.12 (m, 3H), 1.04 (s, 3H), 0.88 (s, 3H).

### Step-2: Synthesis of {2-[({3-[(3R)-3-(ethoxymethyl)-4,4-dimethylcyclohexyl]-1H-pyrazol-4-yl}methyl)(methyl) amino]ethyl}(methyl)amine

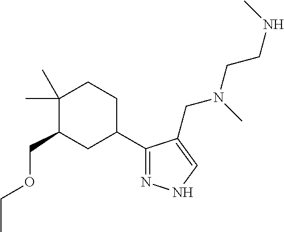 [SMILES: CCOC[C@@H]1CC(c2n[nH]cc2CN(C)CCNC)CCC1(C)C]

HCl (6N, 0.8 ml) was added to a solution of tert-butyl N-{2-[({3-[(3R)-3-(ethoxymethyl)-4,4-dimethylcyclohexyl]-1-(oxan-2-yl)-1H-pyrazol-4-yl}methyl)(methyl)amino]ethyl}-N-methylcarbamate (93%, 180 mg, 0.32 mmol) in dioxane (5 ml). The reaction was stirred 16 h at rt. The solvent was removed under reduced pressure; the residue was solubilised in MeOH (3 ml) and loaded onto an SCX-2 column (5 g). The column was washed with MeOH/DCM (1/1, 20 ml) and the compound was released with 7N NH₃ in MeOH/DCM (1/1, 20 ml). The compound rich fractions were combined to afford 108 mg of {2-[({3-[(3R)-3-(ethoxymethyl)-4,4-dimethylcyclohexyl]-1H-pyrazol-4-yl}methyl)(methyl)amino] ethyl}(methyl) amine (94%) as a yellow oil. LC/MS 2.28 min; m/z=337.20 [M+1]; ¹H NMR (500 MHz, Methanol-d4) δ: 7.42 (s, 1H), 3.69-3.57 (m, 1H), 3.56-3.37 (m, 4H), 3.12 (t, J=8.9 Hz, 1H), 2.83-2.71 (m, 1H), 2.67 (t, J=6.5 Hz, 2H), 2.51 (t, J=6.1 Hz, 2H), 2.39-2.32 (m, 3H), 2.23-2.15 (m, 3H), 2.00-1.90 (m, 1H), 1.90-1.71 (m, 1H), 1.71-1.60 (m, 1H), 1.60-1.35 (m, 4H), 1.24-1.12 (m, 3H), 1.04 (s, 3H), 0.94 (s, 3H).

### Example B-5. Synthesis of [(1R)-2,2-dimethyl-5-[4-({methyl[2-(methylamino)ethyl]amino} methyl)-1H-pyrazol-3-yl]cyclohexyl]methanol (Compound 32)

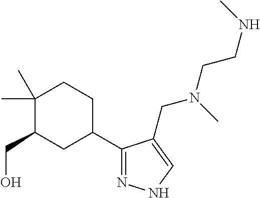 [SMILES: CNCCN(C)Cc1c[nH]nc1C1CCC(C)(C)[C@H](CO)C1]

### Step-1: Synthesis of [(1R)-2,2-dimethyl-5-[4-({methyl[2-(methylamino)ethyl]amino}methyl)-1H-pyrazol-3-yl]cyclohexyl]methanol

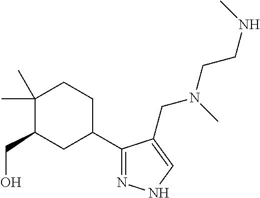 [SMILES: CNCCN(C)Cc1c[nH]nc1C1CCC(C)(C)[C@H](CO)C1]

HCl (6N, 0.4 ml) was added to a solution of tert-butyl N-{2-[({3-[(3R)-3-(hydroxymethyl)-4,4-dimethylcyclohexyl]-1-(oxan-2-yl)-1H-pyrazol-4-yl}methyl)(methyl)amino]ethyl}-N-methylcarbamate (50 mg, 0.1 mmol) in dioxane (2.5 ml). The reaction was stirred at rt 16 h. The solvent was removed under reduced pressure; the residue was solubilised in MeOH (3 ml) and loaded onto an SCX-2 column (2 g). The column was washed with MeOH/DCM (1/1, 20 ml) and the compound was released with 7N NH₃ in MeOH/DCM (1/1, 20 ml). The compound rich fractions were combined to afford 25 mg of [(1R)-2,2-dimethyl-5-[4-({methyl[2-(methylamino)ethyl]amino} methyl)-1H-pyrazol-3-yl]cyclohexyl]methanol (77%) as a colourless oil. LCMS: 1.76 min; m/z=309.10 [M+1]; ¹H NMR (500 MHz, Methanol-d4) δ: 7.42 (s, 1H), 3.86-3.76 (m, 1H), 3.48-3.38 (m, 2H), 3.22 (dd, J=10.6, 8.7 Hz, 1H), 2.76 (ddd, J=12.2, 8.6, 3.7 Hz, 1H), 2.67 (t, J=6.5 Hz, 2H), 2.51 (t, J=6.4 Hz, 2H), 2.36 (s, 3H), 2.27-2.14 (m, 3H), 2.10-1.92 (m, 1H), 1.78 (qd, J=13.1, 3.8 Hz, 1H), 1.72-1.59 (m, 1H), 1.54-1.38 (m, 4H), 1.09 (s, 3H), 0.93 (s, 3H).

### Example B-6. Synthesis of {2-[({3-[(3R)-3-[(2-methoxyethoxy)methyl]-4,4-dimethylcyclohexyl]-1H-pyrazol-4-yl} methyl)(methyl)amino]ethyl}(methyl)amine (Compound 35)

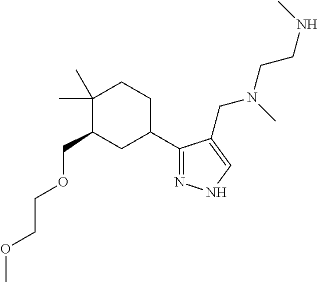 [SMILES: CNCCN(C)Cc1c[nH]nc1C1CCC(C)(C)[C@H](COCCOC)C1]

### Step-1: Synthesis of tert-butyl N-{2-[({3-[(3R)-3-[(2-methoxyethoxy)methyl]-4,4-dimethylcyclohexyl]-1-(oxan-2-yl)-1H-pyrazol-4-yl}methyl) (methyl)amino]ethyl}-N-methylcarbamate

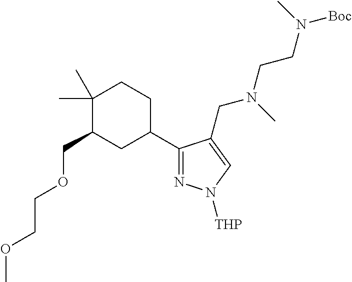

At rt, sodium hydride (60%, 100 mg, 2.54 mmol) was added to a solution of tert-butyl N-{2-[({3-[(3R)-3-(hydroxymethyl)-4,4-dimethylcyclohexyl]-1-(oxan-2-yl)-1H-pyrazol-4-yl}methyl)(methyl)amino] ethyl}-N-methylcarbamate (0.25 g, 0.51 mmol) in THF (10 ml). The solution was stirred 30 min before adding 1-bromo-2-methoxyethane (0.07 g, 0.51 mmol). After ON, LCMS showed 24% TM and 62% SM. A further sodium hydride (60%, 100 mg, 2.54 mmol) and 1-bromo-2-methoxyethane (71 mg, 0.51 mmol) were added and the reaction was continued. After a second 16 h, LCMS showed 68%+11% TM (possible isomers) and 16% SM. The reaction was continued for 2 h before quenching with NH₄Cl (saturated, 20 ml). The solution was extracted with EtOAc (3×30 ml). The combined organic layers were dried over Na₂SO₄ and evaporated to dryness and the residue was purified by Biotage over SiO₂ (SNAP 25 g, eluent EtOAc/MeOH 97/3 to 70/30) to afford 177 mg of tert-butyl N-{2-[({3-[(3R)-3-[(2-methoxyethoxy)methyl]-4,4-dimethylcyclohexyl]-1-(oxan-2-yl)-1H-pyrazol-4-yl}methyl)methyl)amino]ethyl}-N-methylcarbamate (63%) as a light yellow oil. LC/MS 1.17 min; m/z=551.20 [M+1]; ¹H NMR (250 MHz, CDCl₃) δ: 7.42 (s, 1H), 5.39-5.23 (m, 1H), 4.11-4.00 (m, 1H), 3.75-3.44 (m, 5H), 3.43-3.19 (m, 6H), 3.17-3.04 (m, 1H), 2.83 (s, 3H), 2.72-2.57 (m, 1H), 2.53-2.41 (m, 2H), 2.20 (s, 3H), 2.06-1.88 (m, 4H), 1.86-1.51 (m, 9H), 1.51-1.27 (m, 10H), 1.03 (s, 3H), 0.88 (s, 3H).

### Step-2: Synthesis {2-[({3-[(3R)-3-[(2-methoxyethoxy)methyl]-4,4-dimethylcyclohexyl]-1H-pyrazol-4-yl} methyl)(methyl)amino]ethyl}(methyl)amine

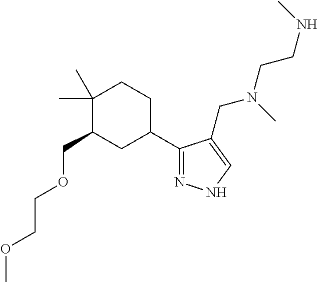 [SMILES: CNCCN(C)Cc1c[nH]nc1C1CCC(C)(C)[C@H](COCCOC)C1]

Tert-butyl N-{2-[({3-[(3R)-3-[(2-methoxyethoxy)methyl]-4,4-dimethylcyclohexyl]-1-(oxan-2-yl)-1H-pyrazol-4-yl}methyl)(methyl)amino]ethyl}-N-methylcarbamate (160 mg, 0.29 mmol) was dissolved in Dioxane (8 ml) and 6M hydrogen chloride (1.28 ml) was added. The reaction was stirred at rt and after 5 h, LCMS showed complete deprotection. The solvent was removed under a flow of nitrogen. The residue was dissolved in DCM/MeOH (1/1, 5 ml) and loaded onto SCX-2 column (5 g). The column was washed with DCM/MeOH (1/1, 50 ml) and the compound was released with 7N NH₃ in MeOH/DCM (1/1, 50 ml). The compound rich fractions were combined to afford 85 mg of {2-[({3-[(3R)-3-[(2-methoxyethoxy)methyl]-4,4-dimethylcyclohexyl]-1H-pyrazol-4-yl}methyl)(methyl)amino]ethyl}(methyl) amine (80%) as an orange oil. LCMS: 2.27 min; m/z 367.15 [M+1]; ¹H NMR (500 MHz, Methanol-d4) δ: 7.42 (s, 1H), 3.70-3.62 (m, 1H), 3.61-3.54 (m, 1H), 3.54-3.48 (m, 3H), 3.42 (s, 2H), 3.33 (s, 3H), 3.17 (t, J=9.0 Hz, 1H), 2.76 (tt, J=12.2, 3.5 Hz, 1H), 2.67 (t, J=6.6 Hz, 2H), 2.54-2.48 (m, 2H), 2.38-2.34 (m, 3H), 2.22-2.17 (m, 3H), 1.99-1.90 (m, 1H), 1.90-1.72 (m, 1H), 1.71-1.62 (m, 1H), 1.62-1.54 (m, 1H), 1.54-1.33 (m, 3H), 1.04 (s, 3H), 0.94 (s, 3H).

### Example B-7. Synthesis of N1-((3-(10-(3-methoxypropoxy)spiro[4.5]dec-6-en-7-yl)-1H-pyrazol-4-yl)methyl)-N1,N2-dimethylethane-1,2-diamine dihydrochloride (Compound 18)

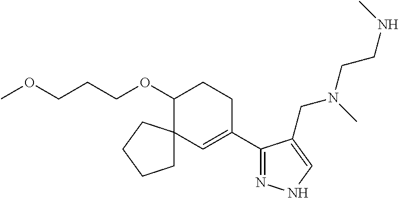 [SMILES: CNCCN(C)Cc1c[nH]nc1C1=CC2(CCCC2)C(OCCCOC)CC1]

### Step-1: Synthesis of 1,4-dioxadispiro[4.1.5⁷.3⁵]pentadecan-13-one

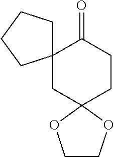 [SMILES: O=C1CCC2(CC13CCCC3)OCCO2]

To a solution of 1,4-dioxaspiro[4.5]decan-8-one (5 g, 32.01 mmol) and 1,5-dibromopentane (7.36 g, 32 mmol) in toluene (120 ml) was added tBuOK (3.59 g, 32 mmol) at rt. The solution was refluxed 16 h. The reaction was cooled to rt and quenched with HCl (0.5N, 10 ml). The phases were separated and the aqueous was extracted with DCM (3×30 ml). The organic layer was dried over Na₂SO₄ and evaporated to dryness. The residue was purified by Biotage (SNAP 340 g, eluent Hep/EtOAc 95/5 to 60/40) to afford 2.35 g of the title compound (33%) as a light colorless oil. ¹H NMR (500 MHz, CDCl₃) δ: 4.08-3.91 (m, 4H), 2.61-2.42 (m, 2H), 2.01-1.95 (m, 2H), 1.93 (s, 2H), 1.83-1.72 (m, 2H), 1.58-1.38 (m, 7H), 1.38-1.28 (m, 1H).

### Step-2: Synthesis of 1,4-dioxadispiro[4.1.5⁷.3⁵]pentadecan-13-ol

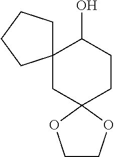 [SMILES: OC1CCC2(CC13CCCC3)OCCO2]

Sodium borohydride (0.99 g, 26.19 mmol) was added at 0° C. and under nitrogen to 1,4-dioxadispiro[4.1.5⁷.3⁵]pentadecan-13-one (2.35 g, 10.48 mmol) in MeOH (120 ml). The reaction stirred at 0° C. until completion (6 h). The reaction was quenched slowly with water (100 ml). DCM (50 ml) was added and the layers separated. The aqueous layer was extracted with DCM (2×50 ml). The organic layers were combined, dried over Na₂SO₄, filtered and vac down to afford 2.18 g of desired 1,4-dioxadispiro[4.1.57.35]pentadecan-13-ol (92%). ¹H NMR (500 MHz, CDCl₃) δ: 3.97-3.86 (m, 4H), 3.52-3.44 (m, 1H), 1.90-1.79 (m, 3H), 1.77-1.62 (m, 2H), 1.61-1.34 (m, 10H), 1.35-1.18 (m, 2H).

### Step-3: Synthesis of 12-(3-methoxypropoxy)-1,4-dioxadispiro[4.1.4⁷.3⁵]tetradecane

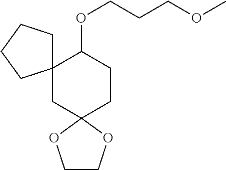 [SMILES: COCCCOC1CCC2(CC13CCCC3)OCCO2]

To a suspension of NaH (60% in oil, 3.01 g, 75.37 mmol) in DMF (150 ml) was added a DMF solution of 1,4-dioxadispiro[4.1.4⁷.3⁵]tetradecan-12-ol (8 g, 37.69 mmol) at room temperature under nitrogen. The reaction mixture was stirred at room temperature for 1 hour and 1-bromo-3-methoxypropane (6.31 ml, 56.53 mmol) was added. The mixture was stirred at room temperature for 6 hours and then it was heated at 70° C. for 24 hours. The reaction was monitored by ¹H-NMR and was stopped before completion. It was cooled at room temperature, quenched with water and extracted with ethyl acetate (3×). The combined organic layers were washed with water (2×) and brine (2×), dried (MgSO₄) and concentrated to give an oil that was purified by Biotage FC using a SNAP KP 340 g column and eluted with Heptane-ethyl acetate 5% to 40% to give 1.13 g (10.5%) of 12-(3-methoxypropoxy)-1,4-dioxadispiro[4.1.4⁷.3⁵]tetradecane as an oil. Starting material was also recovered: 6 g (75%) of 1,4-dioxadispiro[4.1.4⁷.3⁵]tetradecan-12-ol. ¹H NMR (500 MHz, CDCl₃) δ: 3.89 (s, 4H), 3.47 (dd, J=6.7, 2.5 Hz, 1H), 1.86-1.73 (m, 3H), 1.72-1.39 (m, 12H).

### Step-4: Synthesis 10-(3-methoxypropoxy)spiro[4.5]decan-7-one

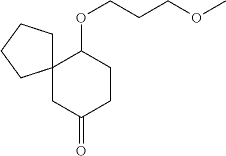 [SMILES: COCCCOC1CCC(=O)CC12CCCC2]

To a solution of 12-(3-methoxypropoxy)-1,4-dioxadispiro[4.1.4⁷.3⁵]tetradecane (1.13 g, 3.97 mmol) in DCM (250 ml) was added iron trichloride hexahydrate (5.37 g, 19.85 mmol) and the resulting suspension was stirred at room temperature for 2 h. The reaction mixture was decanted and washed with aqueous saturated NaHCO₃ (1×), water (1×), brine (1×), dried over MgSO₄, filtered and the filtrate was concentrated in vacuo to give 0.89 g (93%) of 10-(3-methoxypropoxy) spiro[4.5]decan-7-one as an oil that was used in the next step without further purification. ¹H NMR (500 MHz, CDCl₃) δ: 3.71 (dt, J=9.1, 6.1 Hz, 1H), 3.49 (td, J=6.3, 1.8 Hz, 2H), 3.45 (dt, J=9.1, 6.2 Hz, 1H), 3.34 (s, 3H), 3.19 (d, J=3.7 Hz, 1H), 2.59 (d, J=13.6 Hz, 1H), 2.55-2.44 (m, 1H), 2.19-2.09 (m, 2H), 2.09-2.03 (m, 1H), 1.90-1.74 (m, 4H), 1.68-1.50 (m, 4H), 1.44-1.37 (m, 1H), 1.37-1.23 (m, 2H).

### Step-5: Synthesis of 10-(3-methoxypropoxy)spiro[4.5]dec-6-en-7-yl trifluoromethanesulfonate

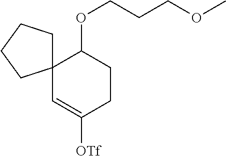 [SMILES: COCCCOC1CCC(OS(=O)(=O)C(F)(F)F)=CC12CCCC2]

10-(3-methoxypropoxy) spiro[4.5]decan-7-one (0.89 g, 3.7 mmol) was dissolved in dry THF (12 ml) and cooled at −78° C. A 1M solution of lithium 1,1,1,3,3,3-hexamethyldisilazan-2-ide in THF (7.4 ml) was added dropwise. The reaction was stirred at −78° C. for 1 h, then allowed to warm to room temperature over 3 h and was quenched by the addition of aqueous saturated NH₄Cl. Ethyl acetate was added, and the organic layer was separated. The aqueous layer was further extracted (2×) and the organics were combined, washed with brine (1×), dried (MgSO₄), filtered, and concentrated under reduced pressure to give an oil that was purified by column chromatography on Biotage, using a SNAP-HP 50 g column, eluted with Heptane-ethyl acetate 5% to 35% to give 1.08 g (78%) of 10-(3-methoxypropoxy)spiro[4.5]dec-6-en-7-yl trifluoromethanesulfonate as an oil. ¹H NMR (500 MHz, CDCl₃) δ: 5.60 (s, 0.9H), 5.52 (s, 0.1H), 3.62 (dt, J=9.1, 6.2 Hz, 1H), 3.50-3.33 (m, 3H), 3.32 (d, J=1.0 Hz, 3H), 1.83-1.68 (m, 3H), 3.19-3.10 (m, 1H), 2.46-2.38 (m, 1H), 2.34-2.28 (m, 2H), 2.06 (d, J=16.8 Hz, 1H), 1.69-1.54 (m, 4H), 1.47-1.33 (m, 3H).

### Step-6: Synthesis of 2-[10-(3-methoxypropoxy)spiro[4.5]dec-6-en-7-yl]-4,4,5,5-tetramethyl-1,3,2-dioxaborolane

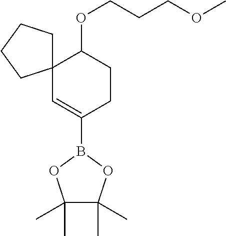 [SMILES: COCCCOC1CCC(B2OC(C)(C)C(C)(C)O2)=CC12CCCC2]

A suspension of 10-(3-methoxypropoxy)spiro[4.5]dec-6-en-7-yl trifluoromethanesulfonate (1.05 g, 2.82 mmol), bis(pinacolato)diboron (0.86 g, 3.38 mmol), potassium acetate (2.08 g, 21.15 mmol) and PdCl₂dppf.DCM (115.13 mg, 0.14 mmol) in 1,4-dioxane (12 ml) was purged with nitrogen for 5 min and then heated at 80° C. 16 h The mixture was allowed to cool at room temperature and diluted with ethyl acetate and filtered through Celite. The filtrate was washed with water (1×), brine (1×) and dried over MgSO₄. The solvent was evaporated and the residue was purified on a 50 g KP Si column on Biotage eluting with a gradient of Heptane:ethyl acetate (5% to 30%) to give 0.73 g (73%) of 2-[10-(3-methoxypropoxy)spiro[4.5]dec-6-en-7-yl]-4,4,5,5-tetramethyl-1,3,2-dioxaborolane as an oil. ¹H NMR (500 MHz, CDCl₃) δ: 6.44 (dq, J=3.5, 1.8 Hz, 0.9H), 6.27 (t, J=1.7 Hz, 0.1H), 3.72-3.56 (m, 1H), 3.50-3.40 (m, 2H), 3.37 (dt, J=9.3, 6.4 Hz, 1H), 3.32 (s, 3H), 3.23-3.13 (m, 1H), 2.36 (dtt, J=18.8, 4.7, 2.1 Hz, 1H), 2.16 (dddd, J=17.4, 9.2, 4.9, 2.7 Hz, 2H), 1.99-1.89 (m, 1H), 1.86-1.71 (m, 2H), 1.71-1.45 (m, 6H), 1.40-1.17 (m, 14H).

### Step-7: Synthesis of tert-butyl N-{2-[({3-[10-(3-methoxypropoxy)spiro[4.5]dec-7-en-7-yl]-1-(oxan-2-yl)-1H-pyrazol-4-yl}methyl)(methyl)amino]ethyl}-N-methylcarbamate

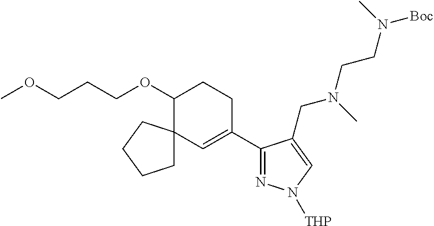

A suspension of 2-[10-(3-methoxypropoxy)spiro[4.5]dec-6-en-7-yl]-4,4,5,5-tetramethyl-1,3,2-dioxaborolane (0.73 g, 2.07 mmol), tert-butyl N-[2-({[3-iodo-1-(oxan-2-yl)-1H-pyrazol-4-yl]methyl} (methyl)amino)ethyl]-N-methylcarbamate (1.19 g, 2.48 mmol) in 1,4-dioxane (10 ml) and aqueous 2M solution of sodium carbonate (3 ml) was degassed by bubbling nitrogen for 5 min. PdCl₂dppf.DCM (85 mg, 0.1 mmol) was added and the reaction was heated at 80° C. for 16 h. The reaction mixture was left to cool at room temperature and then diluted with ethyl acetate, filtered through Celite and the solids were washed with ethyl acetate. The combined filtrates were washed with water (1×) and brine (2×), dried (MgSO₄), filtered and concentrated in vacuo to give an oil. Purification by chromatography on a Biotage using a 100 g SNAP KP column and elution with DCM-MeOH 0 to 10% gave 1.19 g (70%) of tert-butyl N-{2-[({3-[10-(3-methoxypropoxy)spiro[4.5]dec-7-en-7-yl]-1-(oxan-2-yl)-1H-pyrazol-4-yl}methyl)(methyl)amino]ethyl}-N-methylcarbamate. LCMS: 1.16 min; m/z=575.30 [M+1]. ¹H NMR (500 MHz, CDCl₃) δ: 7.55-7.38 (m, 1H), 6.00 (d, J=39.9 Hz, 1H), 5.31 (dt, J=9.1, 2.7 Hz, 1H), 4.06 (d, J=8.1 Hz, 1H), 3.76-3.59 (m, 2H), 3.52-3.43 (m, 2H), 3.44-3.35 (m, 3H), 3.35-3.30 (m, 4H), 3.30-3.21 (m, 2H), 2.87-2.76 (m, 3H), 2.59-2.40 (m, 4H), 2.35-2.18 (m, 4H), 2.03 (d, J=11.0 Hz, 4H), 1.80 (p, J=6.3 Hz, 2H), 1.73-1.52 (m, 11H), 1.44 (s, 9H).

### Step-8: Synthesis of N1-((3-(10-(3-methoxypropoxy)spiro[4.5]dec-6-en-7-yl)-1H-pyrazol-4-yl)methyl)-N1,N2-dimethylethane-1,2-diamine dihydrochloride

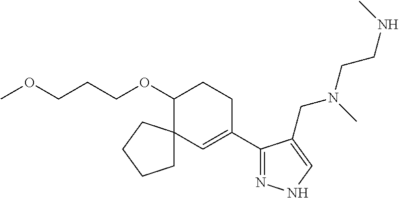 [SMILES: CNCCN(C)Cc1c[nH]nc1C1=CC2(CCCC2)C(OCCCOC)CC1]

To a solution of tert-butyl N-{2-[({3-[10-(3-methoxypropoxy)spiro[4.5]dec-7-en-7-yl]-1-(oxan-2-yl)-1H-pyrazol-4-yl}methyl)(methyl)amino]ethyl}-N-methylcarbamate (42 mg, 0.07 mmol) in MeOH (0.5 ml) was added aqueous 6M HCl (0.25 ml). The reaction mixture was stirred at room temperature for 2 h. It was concentrated to dryness on a rotary evaporator. The residue was purified by prep HPLC using a low pH method (Waters 2). During evaporation, residual formic acid reacted to give the N-formamide as evidenced by NMR and LCMS after evaporation. This was hydrolysed under acidic conditions by heating a 1:1 MeOH-aqueous HCl 6N solution at 60° C. for 3 h. After concentrating and drying under vacuum, we obtained 15 mg (44.3%) of ({3-[10-(3-methoxypropoxy) spiro[4.5]dec-7-en-7-yl]-1H-pyrazol-4-yl}methyl)(methyl)[2-(methylamino)ethyl]amine dihydrochloride as a 9:1 mixture of 2 isomers. LCMS: 2.49 min; m/z=391.15 [M+1]; ¹H NMR (500 MHz, Methanol-d₄) δ: 8.59 (s, 0.1H), 8.54-8.49 (m, 0.9H), 6.22 (s, 0.86H), 6.07 (s, 0.14H), 4.56 (s, 2H), 3.78-3.56 (m, 5H), 3.52-3.44 (m, 3H), 3.36 (q, J=5.6, 4.1 Hz, 1H), 3.33 (d, J=2.5 Hz, 3H), 2.97-2.92 (m, 0.42H), 2.87 (s, 2.58H), 2.80 (s, 3H), 2.62-2.40 (m, 2H), 2.26 (d, J=16.8 Hz, 1H), 2.00 (ddd, J=32.0, 15.1, 9.7 Hz, 1H), 1.81 (qt, J=10.1, 5.0 Hz, 3H), 1.76-1.63 (m, 4H), 1.65-1.41 (m, 3H).

### Example B-8. Synthesis of N1-((3-(10-(3-methoxypropoxy)spiro[4.5]decan-7-yl)-1H-pyrazol-4-yl)methyl)-N1,N2-dimethylethane-1,2-diamine dihydrochloride (Compound 19)

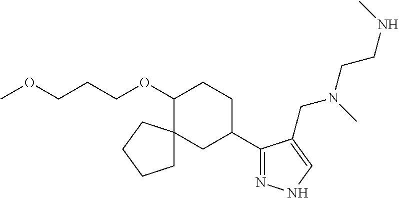 [SMILES: CNCCN(C)Cc1c[nH]nc1C1CCC(OCCCOC)C2(CCCC2)C1]

### Step-1: Synthesis of tert-butyl (2-(((3-(10-(3-methoxypropoxy)spiro[4.5]decan-7-yl)-1-(tetrahydro-2H-pyran-2-yl)-1H-pyrazol-4-yl)methyl)(methyl)amino)ethyl)(methyl)carbamate

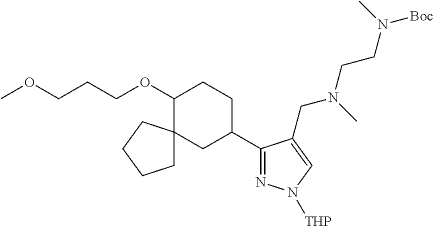

A suspension of tert-butyl N-{2-[({3-[10-(3-methoxypropoxy)spiro[4.5]dec-7-en-7-yl]-1-(oxan-2-yl)-1H-pyrazol-4-yl}methyl)(methyl)amino]ethyl}-N-methylcarbamate (1.19 g, 2.07 mmol) and Raney Ni (3.67 ml, 41.4 mmol) in EtOH (10 ml) was stirred under hydrogen at room temperature and atmospheric pressure for 16 h. The solution was filtered through Celite and the pad was washed with ethyl acetate. The filtrate was concentrated under reduced pressure to give an oil, 877 mg (73%) of tert-butyl N-{2-[({3-[10-(3-methoxypropoxy)spiro[4.5]decan-7-yl]-1-(oxan-2-yl)-1H-pyrazol-4-yl}methyl)(methyl)amino] ethyl}-N-methylcarbamate. This material was used in the next step without further purification. LC/MS 1.43 min; m/z 577.65 [M+1]; ¹H NMR (500 MHz, CDCl₃) δ: 7.41 (s, 1H), 5.33-5.21 (m, 1H), 4.08-3.97 (m, 1H), 3.71-3.63 (m, 1H), 3.60 (dt, J=9.1, 5.9 Hz, 1H), 3.54-3.43 (m, 2H), 3.40-3.20 (m, 8H), 2.96 (s, 1H), 2.80 (d, J=19.9 Hz, 4H), 2.46 (s, 2H), 2.20 (d, J=7.1 Hz, 3H), 2.02-1.76 (m, 9H), 1.74-1.48 (m, 11H), 1.43 (s, 11H).

### Step-2: Synthesis of N1-((3-(10-(3-methoxypropoxy)spiro[4.5]decan-7-yl)-1H-pyrazol-4-yl)methyl)-N1,N2-dimethylethane-1,2-diamine dihydrochloride

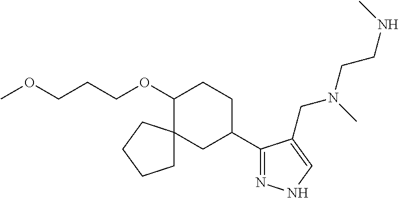 [SMILES: CNCCN(C)Cc1c[nH]nc1C1CCC(OCCCOC)C2(CCCC2)C1]

To a solution of tert-butyl N-{2-[({3-[10-(3-methoxypropoxy)spiro[4.5]decan-7-yl]-1-(oxan-2-yl)-1H-pyrazol-4-yl}methyl)(methyl)amino]ethyl}-N-methylcarbamate (120 mg, 0.21 mmol) in MeOH (1 ml) was added a 6M aqueous solution of HCl (0.71 ml) and the resulting mixture was stirred for 1 h at room temperature. The solvents were removed in vacuo to afford 33 mg (37%) of ({3-[10-(3-methoxypropoxy)spiro[4.5]decan-7-yl]-H-pyrazol-4-yl}methyl)(methyl)[2-(methylamino)ethyl]amine dihydrochloride as a ca. 95:5 mixture of cis and trans isomers, evidenced by the NMR. No assignment of stereochemistry was attempted at this point. LCMS: 2.47 min; m/z=393.2 [M+1]; ¹H NMR (500 MHz, Methanol-d₄) δ: 7.42 (s, 1H), 3.66 (dt, J=9.2, 6.0 Hz, 1H), 3.54 (t, J=6.4 Hz, 2H), 3.42 (s, 2H), 3.40-3.32 (m, 4H), 3.05 (s, 1H), 2.97-2.87 (m, 1H), 2.73 (t, J=6.5 Hz, 2H), 2.52 (t, J=6.5 Hz, 2H), 2.39 (s, 3H), 2.22 (s, 3H), 2.03 (dt, J=14.1, 3.0 Hz, 1H), 1.92 (t, J=12.9 Hz, 1H), 1.88-1.76 (m, 4H), 1.70-1.51 (m, 7H), 1.48-1.32 (m, 3H).

### Example B-9. Synthesis of N1-((3-((7R,10S)-10-(3-methoxypropoxy)spiro[4.5]decan-7-yl)-1H-pyrazol-4-yl)methyl)-N1,N2-dimethylethane-1,2-diamine and N1-((3-((7R,10R)-10-(3-methoxypropoxy)spiro[4.5]decan-7-yl)-1H-pyrazol-4-yl)methyl)-N1,N2-dimethylethane-1,2-diamine (Compound 27 and Compound 26)

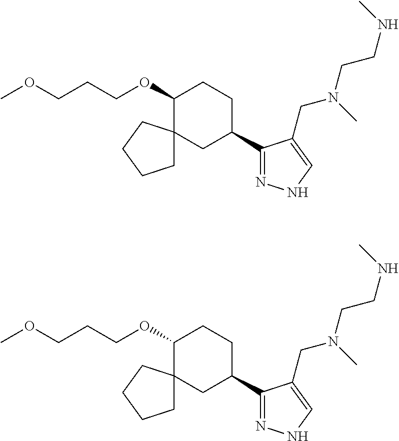 [SMILES: CNCCN(C)Cc1c[nH]nc1[C@@H]1CC[C@H](OCCCOC)C2(CCCC2)C1 ; CNCCN(C)Cc1c[nH]nc1[C@@H]1CC[C@@H](OCCCOC)C2(CCCC2)C1]

A solution of tert-butyl N-{2-[({3-[10-(3-methoxypropoxy)spiro[4.5]decan-7-yl]-1-(oxan-2-yl)-1H-pyrazol-4-yl}methyl)(methyl)amino]ethyl}-N-methylcarbamate (384 mg, 0.67 mmol) in aqueous 6N HCl (4 ml) was stirred at rt for 1 hand then concentrated in vacuo to give an oil. This was purified by chiral prep HPLC (20% Ethanol+0.1% DEA: 80% CO₂ with Chiralpak IC 25 cm at 15 ml/min) to give two products:

({3-[(7,10-cis)-10-(3-methoxypropoxy)spiro[4.5]decan-7-yl]-H-pyrazol-4-yl}methyl)(methyl)[2-(methylamino)ethyl]amine: 80 mg (26%) of a racemic mixture of (7R, 10S) and (7S, 10R) enantiomers. LCMS: 5.03 min; m/z=393.3 [M+1]. ¹H NMR (500 MHz, Methanol-d₄) δ: 7.41 (s, 1H), 3.66 (dt, J=9.2, 6.0 Hz, 1H), 3.55 (t, J=6.4 Hz, 2H), 3.41 (s, 2H), 3.39-3.32 (m, 4H), 3.05 (s, 1H), 2.93 (td, J=12.7, 11.2, 6.5 Hz, 1H), 2.67 (t, J=6.6 Hz, 2H), 2.50 (t, J=6.6 Hz, 2H), 2.35 (s, 3H), 2.21 (s, 3H), 2.06-1.98 (m, 1H), 1.96-1.88 (m, 1H), 1.87-1.74 (m, 4H), 1.70-1.49 (m, 7H), 1.48-1.31 (m, 3H).

({3-[(7,10-trans)-10-(3-methoxypropoxy)spiro[4.5]decan-7-yl]-1H-pyrazol-4-yl}methyl) (methyl)[2-(methylamino)ethyl]amine: 31 mg (10%) of a racemic mixture of (7R, 10R) and (7S, 10S) enantiomers. Analysis showed this product to be 82% pure and contaminated with 14% of the cis isomer. LCMS: 4.9 min, m/z=393.3 [M+1] (also shows 14% of the cis isomer at 5.51 min); ¹H NMR (500 MHz, Methanol-d₄) δ: 7.41 (s, 1H), 3.71 (dt, J=9.2, 6.0 Hz, 1H), 3.57-3.52 (m, OH), 3.49 (dtt, J=9.4, 6.3, 3.0 Hz, 2H), 3.44-3.36 (m, 3H), 3.33 (s, 3H), 3.18-3.10 (m, 1H), 2.90 (td, J=11.0, 9.4, 6.4 Hz, 1H), 2.68 (t, J=6.5 Hz, 2H), 2.56-2.46 (m, 2H), 2.35 (s, 3H), 2.20 (s, 3H), 2.08 (dt, J=10.7, 3.5 Hz, 1H), 1.96-1.75 (m, 5H), 1.73-1.48 (m, 7H), 1.44 (dt, J=12.2, 6.1 Hz, 1H), 1.39-1.27 (m, 2H), 1.22 (td, J=12.4, 11.9, 6.7 Hz, 1H).

### Example B-10. Synthesis of methyl([2-[methyl([3-[(6s,9r)-1-oxaspiro[5.5]undecan-9-yl]-1H-pyrazol-4-yl]methyl)amino]ethyl])amine bistrifluoroacetic acid salt) (Compound 127)

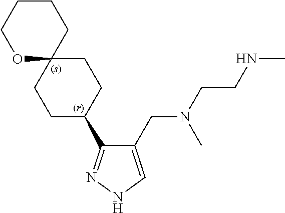 [SMILES: CNCCN(C)Cc1c[nH]nc1[C@H]1CC[C@]2(CCCCO2)CC1]

### Step-1: Synthesis of (but-3-yn-1-yloxy)(tert-butyl)dimethylsilane

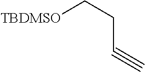

Into a 500 ml round-bottom flask purged and maintained with an inert atmosphere of nitrogen, was placed but-3-yn-1-ol (15 g, 214.01 mmol, 1.00 equiv.), DMF (200 ml), tert-butyl(chloro)dimethylsilane (39 g, 258.75 mmol, 1.21 equiv.), 1H-imidazole (43.7 g, 641.92 mmol, 3.00 equiv.). The resulting solution was stirred at room temperature for 16 h. The resulting solution was diluted with 250 ml of EA. The resulting mixture was washed with 6×150 ml of brine. The mixture was dried over anhydrous Na₂SO₄ and concentrated under vacuum. This resulted in 43 g of (but-3-yn-1-yloxy)(tert-butyl)dimethylsilane as a colorless liquid (100%). ¹H-NMR (300 MHz, CDCl₃) δ: 3.72 (t, J=6.9 Hz, 2H), 2.41-2.35 (m, 2H), 1.95-1.93 (m, 1H), 0.88 (s, 9H), 0.06 (s, 6H).

### Step-2: Synthesis of 8-[4-[(tert-butyldimethylsilyl)oxy]but-1-yn-1-yl]-1,4-dioxaspiro[4.5]decan-8-ol

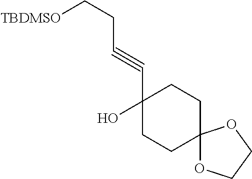 [SMILES: CC(C)(C)[Si](C)(C)OCCC#CC1(O)CCC2(CC1)OCCO2]

Into a 250 ml 3-necked round-bottom flask purged and maintained with an inert atmosphere of nitrogen, was placed (but-3-yn-1-yloxy)(tert-butyl)dimethylsilane (13 g, 70.52 mmol, 1.10 equiv.), tetrahydrofuran (100 ml). This was followed by the addition of n-BuLi (28 ml, 2.5M in hexane) dropwise with stirring at −78° C. The resulting solution was stirred at −35° C. for 1 h. To this was added a solution of 1,4-dioxaspiro[4.5]decan-8-one (10 g, 64.03 mmol, 1.00 equiv.) in tetrahydrofuran (50 ml) dropwise with stirring at −78° C. The resulting solution was allowed to react, with stirring, for an additional 3 h at −78° C. to room temperature. The reaction was then quenched by the addition of 10 ml of MeOH. The resulting mixture was concentrated under vacuum. The resulting solution was diluted with 300 ml of EA. The resulting mixture was washed with 3×150 ml of brine. The mixture was dried over anhydrous sodium sulfate and concentrated under vacuum. The residue was applied onto a silica gel column with ethyl acetate/petroleum ether (3:100˜10:100). This resulted in 15 g of the title compound as a yellow liquid (69%). ¹H-NMR (300 MHz, CDCl₃) δ: 3.87 (s, 4H), 3.62 (t, J=6.9 Hz, 2H), 2.3 (t, J=7.2 Hz, 2H), 1.86-1.70 (m, 8H), 0.83 (s, 9H), 0.01 (s, 6H).

### Step-3: Synthesis of 8-[4-[(tert-butyldimethylsilyl)oxy]butyl]-1,4-dioxaspiro[4.5]decan-8-ol

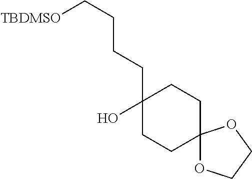 [SMILES: CC(C)(C)[Si](C)(C)OCCCCC1(O)CCC2(CC1)OCCO2]

Into a 500 ml round-bottom flask, was placed 8-[4-[(tert-butyldimethylsilyl)oxy]but-1-yn-1-yl]-1,4-dioxaspiro[4.5]decan-8-ol (14 g, 41.11 mmol, 1.00 equiv), methanol (150 ml), 10% Palladium carbon (1.4 g). To the above hydrogen was introduced in and maintained at 2 atm pressure. The resulting solution was stirred at room temperature for 16 h. The solids were filtered out. The resulting mixture was concentrated under vacuum. This resulted in 13.3 g of 8-[4-[(tert-butyldimethylsilyl)oxy]butyl]-1,4-dioxaspiro[4.5]decan-8-ol as colorless oil (94%). ¹H-NMR (300 MHz, CDCl₃) δ: 3.85 (s, 4H), 1.90-1.75 (m, 2H), 1.62-1.20 (m, 14H), 0.85 (s, 9H), 0.07-0.01 (m, 6H).

### Step-4: Synthesis of 8-(4-hydroxybutyl)-1,4-dioxaspiro[4.5]decan-8-ol

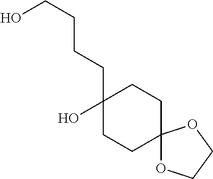 [SMILES: OCCCCC1(O)CCC2(CC1)OCCO2]

Into a 250-mL round-bottom flask, was placed 8-[4-[(tert-butyldimethylsilyl)oxy]butyl]-1,4-dioxaspiro[4.5]decan-8-ol (13.3 g, 38.60 mmol, 1.00 equiv.), tetrahydrofuran (50 ml), TBAF (1M/L in THF 38.6 ml). The resulting solution was stirred at room temperature for 48 h. The resulting mixture was concentrated under vacuum. The residue was applied onto a silica gel column with ethyl acetate/petroleum ether (1:4-4:1). This resulted in 4.82 g (54%) of 8-(4-hydroxybutyl)-1,4-dioxaspiro[4.5]decan-8-ol as a off-white solid. ¹H-NMR (300 MHz, CDCl₃) δ: 4.01-3.89 (m, 4H), 3.72-3.60 (m, 2H), 1.98-1.80 (m, 2H), 1.75-1.60 (m, 12H).

### Step-5: Synthesis of 1-oxaspiro[5.5]undecan-9-one ethylene ketal

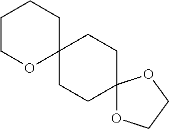 [SMILES: C1CCC2(CCC3(CC2)OCCO3)OC1]

Into a 100 ml round-bottom flask, was placed 8-(4-hydroxybutyl)-1,4-dioxaspiro[4.5]decan-8-ol (2 g, 8.68 mmol, 1.00 equiv.), benzene (20 ml), CMBP (3.14 g). The resulting solution was stirred for 1 for 16 h at 90° C. The resulting mixture was concentrated under vacuum. The residue was applied onto a silica gel column with ethyl acetate/petroleum ether (5:95). This resulted in 1.4 g of the title compound as light yellow oil (76%). ¹H-NMR (300 MHz, CDCl₃) δ: 4.01-3.89 (m, 4H), 3.70-3.55 (m, 2H), 2.01-1.90 (m, 2H), 1.90-1.78 (m, 2H), 1.69-1.40 (m, 10H).

### Step-6: Synthesis of 1-oxaspiro[5.5]undecan-9-one

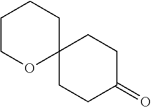 [SMILES: O=C1CCC2(CCCCO2)CC1]

Into a 100 ml round-bottom flask, was placed 1-oxaspiro[5.5]undecan-9-one ethylene ketal (1.4 g, 6.59 mmol, 1.00 equiv.), FeCl₃-6H₂O (5.35 g), dichloromethane (20 ml). The resulting solution was stirred for 2 h at room temperature. The reaction was then quenched by the addition of 10 ml of sodium bicarbonate (sat. aq.). The resulting solution was extracted with 3×20 ml of dichloromethane and the organic layers combined. The resulting mixture was washed with 3×10 ml of brine. The mixture was dried over anhydrous sodium sulfate and concentrated under vacuum. This resulted in 0.94 g of 1-oxaspiro[5.5]undecan-9-one as colorless oil (85%). ¹H-NMR (300 MHz, CDCl₃) δ: 3.80-3.61 (m, 2H), 2.72-2.51 (m, 2H), 2.40-2.10 (m, 4H), 1.75-1.60 (m, 4H), 1.60-1.50 (m, 4H).

### Step-7: Synthesis of oxaspiro[5.5]undec-8-en-9-yl trifluoromethanesulfonate

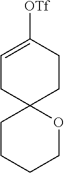 [SMILES: O=S(=O)(OC1=CCC2(CCCCO2)CC1)C(F)(F)F]

Into a 100 ml 3-necked round-bottom flask, was placed 1-oxaspiro[5.5]undecan-9-one (940 mg, 5.77 mmol, 1.00 equiv.), oxolane (10 ml), to the above was added LiHMDS (1M in THF, 6.7 ml) at −78° C., stirred at −40° C. for 1 h, then was added 1,1,1-trifluoro-N-phenyl-N-(trifluoromethane)sulfonylmethane sulfonamide (2.2 g, 6.16 mmol, 1.07 equiv.) at −78° C. The resulting solution was stirred for 3 h at room temperature. The reaction was then quenched by the addition 10 ml of water. The resulting mixture was concentrated under vacuum. The resulting solution was extracted with 3×100 ml of dichloromethane and the organic layers combined and dried over anhydrous sodium sulfate and concentrated under vacuum. The residue was applied onto a silica gel column with ethyl acetate/petroleum ether (0:100-3:97). This resulted in 1.2 g of the tittle compound as light yellow oil (69%). ¹H-NMR (300 MHz, CDCl₃) δ: 5.72-5.50 (s, 1H), 3.80-3.51 (m, 2H), 2.55-2.20 (m, 5H), 1.80-1.30 (m, 7H).

### Step-8: Synthesis of 4,4,5,5-tetramethyl-2-[1-oxaspiro[5.5]undec-8-en-9-yl]-1,3,2-dioxaborolane

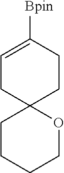

Into a 100 ml round-bottom flask, was placed 1-oxaspiro[5.5]undec-8-en-9-yl trifluoromethanesulfonate (1.2 g, 4.00 mmol, 1.00 equiv.), Pd(dppf)Cl₂ (290 mg, 0.40 mmol, 0.10 equiv.), 4,4,5,5-tetramethyl-2-(tetramethyl-1,3,2-dioxaborolan-2-yl)-1,3,2-dioxaborolane (1.22 g, 4.80 mmol, 1.20 equiv.), potassium acetate (1.2 g, 12.23 mmol, 3.06 equiv.) and dioxane (12 ml). The resulting solution was stirred for 8 h at 80° C. The resulting mixture was concentrated under vacuum. The residue was applied onto a silica gel column with ethyl acetate/petroleum ether (1:0-1:10). This resulted in 0.83 g of the title compound as light yellow oil (75%). ¹H-NMR (300 MHz, CDCl₃) δ: 6.48 (s, 1H), 3.70 (t, J=5.7 Hz, 2H), 2.25-2.10 (m, 4H), 2.02-1.90 (m, 1H), 1.75-1.60 (m, 2H), 1.60-1.40 (m, 4H), 1.30 (s, 12H).

### Step-9: Synthesis of tert-butyl N-methyl-N-[2-[methyl([[1-(oxan-2-yl)-3-[1-oxaspiro[5.5]undec-8-en-9-yl]-1H-pyrazol-4-yl]methyl])amino]ethyl]carbamate

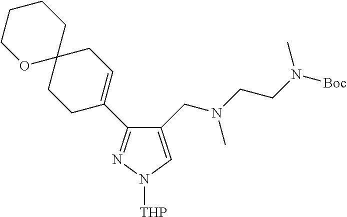

Into a 100 ml round-bottom flask purged and maintained with an inert atmosphere of nitrogen, was placed 4,4,5,5-tetramethyl-2-[1-oxaspiro[5.5]undec-8-en-9-yl]-1,3,2-dioxaborolane (830 mg, 2.98 mmol, 1.00 equiv.), Pd(dppf)Cl₂ (220 mg, 0.30 mmol, 0.10 equiv.), tert-butyl N-[2-([3-iodo-1-(oxan-2-yl)-1H-pyrazol-4-yl]methyl(methyl)amino)ethyl]-N-methylcarbamate (1.14 g, 2.38 mmol, 0.80 equiv.), potassium methaneperoxoate (1.24 g, 8.91 mmol, 2.99 equiv.), dioxane (10 ml), water (1 ml). The resulting solution was stirred at 100° C. for 16 h. The resulting mixture was concentrated under vacuum. The resulting solution was diluted with 20 mL of H₂O. The resulting solution was extracted with 3×100 ml of dichloromethane and the organic layers combined and dried over anhydrous sodium sulfate and concentrated under vacuum. The residue was applied onto a silica gel column with ethyl acetate/petroleum ether (1:4-1:1). This resulted in 560 mg of the title compound as light brown oil (37%). LCMS: 1.31 min, m/z=503.4 [M+1].

### Step-10: Synthesis of tert-butyl N-methyl-N-[2-[methyl([[1-(oxan-2-yl)-3-[1-oxaspiro[5.5]undecan-9-yl]-1H-pyrazol-4-yl]methyl])amino]ethyl]carbamate

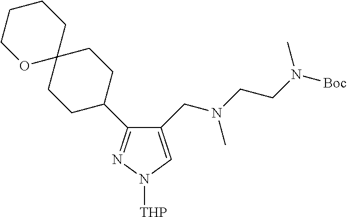

Into a 100 ml round-bottom flask, was placed tert-butyl N-methyl-N-[2-[methyl([[1-(oxan-2-yl)-3-[1-oxaspiro[5.5]undec-8-en-9-yl]-1H-pyrazol-4-yl]methyl])amino]ethyl]carbamate (560 mg, 1.11 mmol, 1.00 equiv), oxolane (10 ml), 10% Pd/C (560 mg, 3.99 mmol, 3.58 equiv.). To the above hydrogen was introduced in and maintained at 2 atm pressure. The resulting solution was stirred for 2 h at room temperature. The solids were filtered out. The resulting mixture was concentrated under vacuum. This resulted in 560 mg of the title compound as a colorless oil (100%). LCMS: 1.17 min, m/z=505.0 [M+1].

### Step-11: Synthesis of methyl([2-[methyl([3-[(6s,9r)-1-oxaspiro[5.5]undecan-9-yl]-1H-pyrazol-4-yl]methyl)amino]ethyl])amine bis(trifluoroacetic acid)

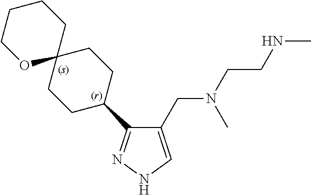 [SMILES: CNCCN(C)Cc1c[nH]nc1[C@H]1CC[C@]2(CCCCO2)CC1]

Into a 50 ml round-bottom flask, was placed tert-butyl N-methyl-N-[2-[methyl([[1-(oxan-2-yl)-3-[1-oxaspiro[5.5]undecan-9-yl]-1H-pyrazol-4-yl]methyl])amino]ethyl]carbamate (100 mg, 0.20 mmol, 1.00 equiv.), trifluoroacetic acid (1 ml), dichloromethane (3 ml). The resulting solution was stirred at room temperature for 16 h. The resulting mixture was concentrated under vacuum. The crude product was purified by Prep-HPLC with the following conditions (waters-1): Column, XBridge; mobile phase, phase A: water with 0.2% TFA; phase B: CH₃CN (10% CH₃CN up to 43% in 10 min, up to 100% in 13 min; Detector, UV 220/254 nm. This resulted in 26.7 mg (25%) of methyl([2-[methyl([3-[(6s,9r)-1-oxaspiro[5.5]undecan-9-yl]-1H-pyrazol-4-yl]methyl)amino]ethyl])amine; bis(trifluoroacetic acid) as colorless oil. LCMS: 1.21 min, m/z=321.1 [M+1-2TFA]. ¹H-NMR (300 MHz, D₂O): δ 8.06 (s, 1H), 4.35 (s, 2H), 3.65-3.35 (m, 6H), 2.99-2.82 (m, 1H), 2.76 (s, 3H), 2.69 (s, 3H), 2.12-1.95 (m, 2H), 1.79-1.20 (m, 12H).

### Example B-11. Synthesis of 3-ethyl-N-(2-methoxyethyl)-2,2-dimethyl-5-[4-([methyl[2-(methylamino)ethyl]amino]methyl)-1H-pyrazol-3-yl]cyclohexane-1-carboxamide dihydrochloride (Compound 21)

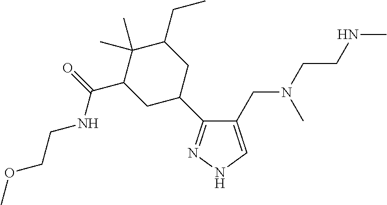

### Step-1: Synthesis of 5-ethyl-4,4-dimethylcyclohex-2-en-1-one

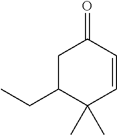

Into a 50 ml 3-necked round-bottom flask, was placed CuI (1.53 g, 8.03 mmol, 2.01 equiv.) and ether (30 ml). EtMgBr (3M in ether, 5.3 ml) was added into mixture at −80° C. After stirred for 10 min, the mixture was allowed to warmed to 0° C., then 4,4-dimethylcyclohexa-2,5-dien-1-one (488 mg, 3.99 mmol, 1.00 equiv.) was added into by dropwise. The resulting solution was stirred for additional 30 min at 0° C. The reaction was quenched by 30 ml of NH₄Cl (sat.). The mixture was washed by 3×30 ml of brine (sat.). The organic phase was collected and dried with anhydrous Na₂SO₄. The crude was concentrated under vacuum. The residue was purified by flash chromatography (PE:EtOAc=10:1) to result in 50 mg (8%) of the title compound as a colorless oil. LCMS: 1.05 min, m/z=153.0 [M+1].

### Step-2: Synthesis of 3-ethyl-2,2-dimethyl-5-oxocyclohexane-1-carbonitrile

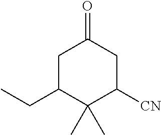

Into a 100 ml round-bottom flask, was placed 5-ethyl-4,4-dimethylcyclohex-2-en-1-one (1.52 g, 9.98 mmol, 1.00 equiv.) and N,N-dimethylformamide (12 ml). Then NH₄Cl (642 mg, 12.00 mmol, 1.20 equiv.) in 12 ml of water was added into by dropwise. Then KCN (780 mg, 11.98 mmol, 1.20 equiv.) in 12 ml of water was added into by dropwise. The resulting solution was stirred at 70° C. for 7 h. The reaction was diluted by 100 mL of EtOAc. The solution was washed by 3×20 ml of FeSO₄ (sat. aq.). Then it was washed again with 50 ml of brine (sat.). The organic phase was concentrated under vacuum and the residue was purified by flash chromatography (PE:EtOAc=5:1) to give 550 mg of the title compound as a colorless oil (31%). LCMS: 1.03 min, m/z=179.2 [M+1].

### Step-3: Synthesis of methyl 3-ethyl-2,2-dimethyl-5-oxocyclohexane-1-carboxylate

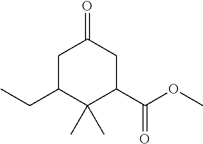

Into a 50 ml round-bottom flask, was placed 3-ethyl-2,2-dimethyl-5-oxocyclohexane-1-carbonitrile (1.5 g, 8.37 mmol, 1.00 equiv.), methanol (40 ml), sulfuric acid (conc. 8 ml). The resulting solution was heated to reflux for 24 hr. The reaction mixture was diluted by 150 ml of EtOAc, and was washed by 3×50 ml of brine (sat.). The organic phase was concentrated and the residue was purified by flash chromatography (PE:EtOAc=5:1) to give 900 mg of the title compound as a colorless oil (51%). ¹H-NMR (300 MHz, CDCl₃) δ 3.82 (s, 3H), 2.80-2.70 (m, 1H), 2.69-2.48 (m, 1H), 2.55-2.45 (m, 2H), 2.15-1.95 (m, 1H), 1.90-1.75 (m, 1H), 1.70-1.55 (m, 1H), 1.15-0.91 (m, 7H), 0.90 (t, J=6.4 Hz, 3H).

### Step-4: Synthesis of methyl 5-ethyl-6,6-dimethyl-3-[(trifluoromethane)sulfonyloxy]cyclohex-2-ene-1-carboxylate

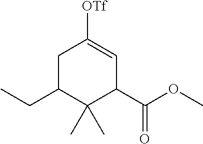 [SMILES: CCC1CC(OS(=O)(=O)C(F)(F)F)=CC(C(=O)OC)C1(C)C]

Into a 50 mL round-bottom flask, was placed methyl 3-ethyl-2,2-dimethyl-5-oxocyclohexane-1-carboxylate (900 mg, 4.24 mmol, 1.00 equiv.), dichloromethane (15 ml), 2,6-di-tert-butyl-4-methylpyridine (1.04 g, 5.06 mmol, 1.19 equiv.). Tf₂O (2.391 g, 8.47 mmol, 2.00 equiv.) was added into by dropwise at 0° C. The resulting solution was heated to reflux for 5 h. The reaction was quenched by 20 ml of water. The organic phase was concentrated and the residue was purified by flash chromatography (PE:EtOAc=10:1) to result in 1.0 g of the title compound as a yellow oil (68%). LCMS: 1.27 min, m/z=345.0 [M+1].

### Step-5: Synthesis of methyl 5-ethyl-6,6-dimethyl-3-(tetramethyl-1,3,2-dioxaborolan-2-yl)cyclohex-2-ene-1-carboxylate

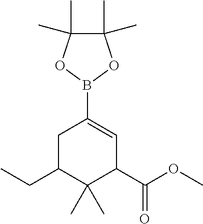 [SMILES: CCC1CC(B2OC(C)(C)C(C)(C)O2)=CC(C(=O)OC)C1(C)C]

Into a 50 ml round-bottom flask, was placed methyl 5-ethyl-6,6-dimethyl-3-[(trifluoromethane) sulfonyloxy]cyclohex-2-ene-1-carboxylate (1 g, 2.90 mmol, 1.00 equiv.), (BPin)₂ (886 mg, 3.49 mmol, 1.20 equiv.), 1,4-dioxane (10 ml), KOAc (855 mg, 8.71 mmol, 3.00 equiv.), PdCl₂dppf (0.64 mg). The resulting solution was heated to reflux for 12 h. The reaction mixture was concentrated and the residue was purified by flash chromatography (PE:EtOAc=10:1) to give 600 mg of the title compound as a yellow oil (64%). LCMS: 1.34 min, m/z=323.0 [M+1].

### Step-6: Synthesis of methyl 3-(4-[[(2-[[(tert-butoxy)carbonyl](methyl)amino]ethyl)(methyl)amino]methyl]-1-(oxan-2-yl)-1H-pyrazol-3-yl)-5-ethyl-6,6-dimethylcyclohex-2-ene-1-carboxylate

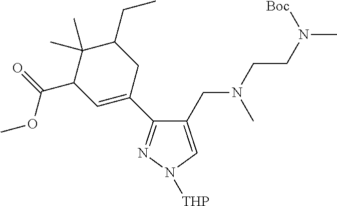

Into a 50 ml round-bottom flask, was placed tert-butyl N-[2-([[3-iodo-1-(oxan-2-yl)-1H-pyrazol-4-yl]methyl](methyl)amino)ethyl]-N-methylcarbamate (250 mg, 0.52 mmol, 1.00 equiv.), methyl 5-ethyl-6,6-dimethyl-3-(tetramethyl-1,3,2-dioxaborolan-2-yl)cyclohex-2-ene-1-carboxylate (200 mg, 0.62 mmol, 1.18 equiv.), 1,4-dioxane (8 ml), H₂O (2 ml), potassium carbonate (207 mg, 1.50 mmol, 2.87 equiv.), PdCl₂.dppf (110 mg). The resulting solution was stirred for 12 h at 100° C. The reaction mixture was concentrated and the residue was purified by flash chromatography using EA as eluent to give 100 mg of the title compound as a brown oil (35%). LCMS: 1.89 min, m/z=547.0 [M+1].

### Step-7: Synthesis of methyl 5-(4-[[(2-[[(tert-butoxy)carbonyl](methyl)amino]ethyl)(methyl) amino]methyl]-1-(oxan-2-yl)-1H-pyrazol-3-yl)-3-ethyl-2,2-dimethylcyclohexane-1-carboxylate

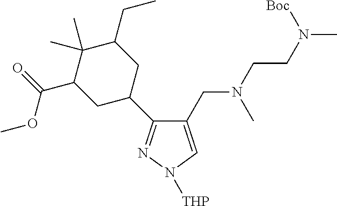

Into a 100-mL round-bottom flask, was placed methyl 3-(4-[[(2-[[(tert-butoxy)carbonyl](methyl) amino]ethyl)(methyl)amino]methyl]-1-(oxan-2-yl)-1H-pyrazol-3-yl)-5-ethyl-6,6-dimethylcyclohex-2-ene-1-carboxylate (180 mg, 0.33 mmol, 1.00 equiv.), tetrahydrofuran (20 ml), 10% Pd(OH)₂/C (360 mg). Then H₂ was introduced into mixture and maintained at 2 atm pressure. The resulting solution was stirred for 2 d at room temperature. The reaction mixture was filtered, the filtrate was concentrated, this resulted in 100 mg (55%) of crude title compound as a light yellow oil. LCMS: 0.96 min, m/z=549.0 [M+1].

### Step-8: Synthesis of sodium 5-(4-[[(2-[[(tert-butoxy)carbonyl](methyl)amino]ethyl)(methyl)amino] methyl]-1-(oxan-2-yl)-1H-pyrazol-3-yl)-3-ethyl-2,2-dimethylcyclohexane-1-carboxylate

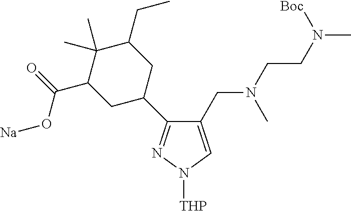

Into a 50-mL round-bottom flask, was placed methyl 5-(4-[[(2-[[(tert-butoxy)carbonyl](methyl) amino]ethyl)(methyl)amino]methyl]-1-(oxan-2-yl)-1H-pyrazol-3-yl)-3-ethyl-2,2-dimethylcyclohexane-1-carboxylate (100 mg, 0.18 mmol, 1.00 equiv.), ethanol (10 ml), sodium hydroxide (219 mg, 5.47 mmol, 30.04 equiv.), water (131 ml). The resulting solution was heated to reflux for 2 days. The reaction mixture was concentrated to get 300 mg of the title compound as the sodium salt that was used directly in the next reaction without further purification (296%). LCMS: 1.36 min, m/z=535.0 [M−Na+1].

### Step-9: Synthesis of tert-butyl N-(2-[[(3-[3-ethyl-5-[(2-methoxyethyl)carbamoyl]-4,4-dimethylcyclohexyl]-1-(oxan-2-yl)-1H-pyrazol-4-yl)methyl](methyl)amino]ethyl)-N-methylcarbamate

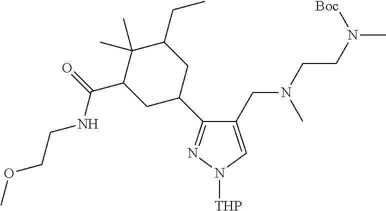

Into a 50-mL round-bottom flask, was placed sodium 5-(4-[[(2-[[(tert-butoxy)carbonyl](methyl) amino]ethyl)(methyl)amino]methyl]-1-(oxan-2-yl)-1H-pyrazol-3-yl)-3-ethyl-2,2-dimethylcyclohexane-1-carboxylate (102 mg, 0.18 mmol, 1.00 equiv.), dichloromethane (10 ml), 2-methoxyethan-1-amine (273 mg, 3.63 mmol, 19.84 equiv.), HATU (555 mg, 1.46 mmol, 7.97 equiv.), TEA (221 mg, 2.18 mmol, 11.92 equiv.). The resulting solution was stirred for 12 h at room temperature. The reaction mixture was diluted by 100 ml of EtOAc. The organic phase was washed by 3×20 ml of brine (sat.). Then the solution was concentrated and the residue was purified by flash chromatography using EtOAc as eluent to give 50 mg of the title compound as yellow oil (46%). LCMS: 1.07 min, m/z=593.0 [M+1].

### Step-10: Synthesis of 3-ethyl-N-(2-methoxyethyl)-2,2-dimethyl-5-[4-([methyl[2-(methylamino)ethyl]amino]methyl)-1H-pyrazol-3-yl]cyclohexane-1-carboxamide dihydrochloride

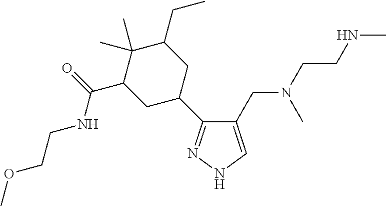

Into a 50 mL round-bottom flask, was placed tert-butyl N-(2-[[(3-[3-ethyl-5-[(2-methoxyethyl)carbamoyl]-4,4-dimethylcyclohexyl]-1-(oxan-2-yl)-1H-pyrazol-4-yl)methyl](methyl)amino]ethyl)-N-methylcarbamate (50 mg, 0.1 mmol, 1.00 equiv.), MeOH (10 ml). Then hydrogen chloride was introduced into mixture. The resulting solution was stirred for 5 h at room temperature. The reaction mixture was concentrated and the residue was purified by Prep-HPLC with the following conditions (Prep-HPLC-025): Column, XBridge Prep C18 OBD Column, 5 um, 19×150 mm; mobile phase, 0.05% TFA in water and MeCN (5.0% MeCN up to 21.0% in 10 min); Detector, UV 254/220 nm. The result solution was acidified by hydrochloric acid (12N), concentrated and dried to give 7.5 mg of the title compound as the dihydrochloride salt as a yellow solid (92%). LCMS: 1.28 min, m/z=408.0 [M+1]. ¹H-NMR (300 MHz, D₂O) δ: 7.85-7.70 (m, 1H), 4.35-4.15 (m, 2H), 3.60-3.20 (m, 11H), 2.90-2.70 (m, 7H), 2.65-2.35 (m, 1H), 2.22-2.15 (m, 0.5H), 2.00-1.45 (m, 4.5H), 1.40-1.10 (m, 2H), 0.95-0.70 (m, 9H).

### Example B-12. Synthesis of N1-ethyl-N3-isopentyl-N1,2,2-trimethyl-5-(4-((methyl(2-(methylamino)ethyl)amino)methyl)-1H-pyrazol-3-yl)cyclohexane-1,3-dicarboxamide (Compound 14)

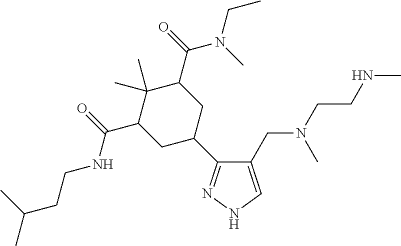 [SMILES: CCN(C)C(=O)C1CC(c2n[nH]cc2CN(C)CCNC)CC(C(=O)NCCC(C)C)C1(C)C]

### Step-1: Synthesis of 8,8-dimethyl-1,4-dioxaspiro[4.5]decane-7,9-dicarboxylic acid

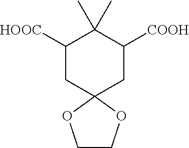 [SMILES: CC1(C)C(C(=O)O)CC2(CC1C(=O)O)OCCO2]

Into a 500 mL round-bottom flask purged and maintained with an inert atmosphere of nitrogen, was placed 8,8-dimethyl-1,4-dioxaspiro[4.5]decane-7,9-dicarbonitrile (14.5 g, 65.83 mmol, 1.00 equiv.), 30% of KOH (150 mL), 30% of H₂O₂ (50 ml). The resulting solution was stirred for 5 days at 110° C. The resulting solution was diluted with 100 ml of NH₄Cl (sat. aq). The resulting solution was extracted with 3×100 ml of ethyl acetate and the organic layers combined and concentrated under vacuum. The crude product was re-crystallized from EA: hexane in the ratio of 2:5. This resulted in 3.9 g of the title compound as a white solid (23%). ¹H-NMR (300 MHz, DMSO-d6) δ: 12.26 (s, 2H), 3.86 (s, 4H), 2.40-2.28 (m, 2H), 1.79 (t, J=13.5 Hz, 2H), 1.62 (d, J=14.7 Hz, 2H), 1.07 (s, 3H), 0.97 (s, 3H).

### Step-2: Synthesis of 7,9-dimethyl 8,8-dimethyl-1,4-dioxaspiro[4.5]decane-7,9-dicarboxylate

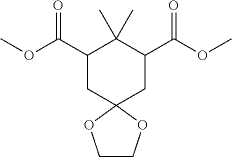

Into a 100 ml round-bottom flask purged and maintained with an inert atmosphere of nitrogen, was placed 8,8-dimethyl-1,4-dioxaspiro[4.5]decane-7,9-dicarboxylic acid (3.5 g, 13.55 mmol, 1.00 equiv.), DMSO (40 ml), the solution was stirred for 15 min, then, KOH (1.68 g, 29.94 mmol, 2.21 equiv.) was added and stirred for 30 min, and iodomethane (7.72 g, 54.39 mmol, 4.01 equiv.) was added. The resulting solution was stirred for 16 h at room temperature. The resulting solution was diluted with 40 ml of ethyl acetate. The resulting mixture was washed with 4×40 ml of brine (sat.). The resulting mixture was concentrated under vacuum. The residue was applied onto a silica gel column with ethyl acetate/petroleum ether (1:10). This resulted in 3 g of the title compound as a white solid (77%). LCMS: 1.42 min, m/z=287.0 [M+1]. ¹H-NMR (300 MHz, CDCl₃) δ: 3.96-3.90 (m, 4H), 3.66 (s, 6H), 2.59 (dd, J=13.5 and 3.3 Hz, 2H), 2.03 (t, J=13.5 Hz, 2H), 1.69 (d, J=15.0 Hz, 2H), 1.07 (s, 3H), 1.05 (s, 3H).

### Step-3: Synthesis of 1,3-dimethyl 2,2-dimethyl-5-oxocyclohexane-1,3-dicarboxylate

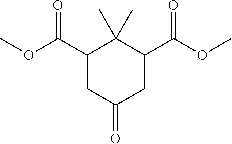

Into a 250 ml round-bottom flask, was placed 7,9-dimethyl 8,8-dimethyl-1,4-dioxaspiro[4.5]decane-7,9-dicarboxylate (3.5 g, 12.22 mmol, 1.00 equiv.), FeCl₃-6H₂O (10.5 g), dichloromethane (50 ml). The resulting solution was stirred for 2 d at room temperature. The resulting solution was allowed to react, with stirring, for an additional 2 d at 45° C. The resulting solution was diluted with 50 ml of water. The resulting solution was extracted with 3×30 ml of dichloromethane and the organic layers combined. The resulting mixture was washed with 3×30 ml of NH₄Cl (sat. aq). The resulting mixture was concentrated under vacuum. This resulted in 3.0 g of the title compound as a brown solid that was used in the next step without further purification. LCMS: 0.76 min, m/z=243.1 [M+1].

### Step-4: Synthesis of 1,3-dimethyl 2,2-dimethyl-5-[(trifluoromethane)sulfonyloxy]cyclohex-4-ene-1,3-dicarboxylate

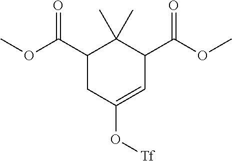

Into a 250 ml round-bottom flask, was placed 1,3-dimethyl 2,2-dimethyl-5-oxocyclohexane-1,3-dicarboxylate (2.8 g, 8.09 mmol, 1.00 equiv.), dichloromethane (40 ml), 2,6-di-tert-butyl-4-methylpyridine (2.0 g, 9.74 mmol, 1.20 equiv.), the solution was cooled down to 0° C. Then Tf₂O (4.57 g, 16.20 mmol, 2.00 equiv.) was added slowly. The resulting solution was stirred for 30 min at 0° C. The resulting solution was allowed to react, with stirring, for an additional for 16 h at room temperature. The resulting solution was diluted with 20 ml of water. The organic layer was concentrated under vacuum. The residue was applied onto a silica gel column with ethyl acetate/petroleum ether (1:2). This resulted in 2.1 g of the title compound as a light yellow solid (69%). ¹H-NMR (300 MHz, CDCl₃) δ: 5.78 (s, 1H), 3.73 (s, 3H), 3.71 (s, 3H), 3.20-3.18 (m, 1H), 2.88-2.80 (m, 1H), 2.78-2.65 (m, 1H), 2.44-2.36 (m, 1H), 1.20 (s, 3H), 1.00 (s, 3H).

### Step-5: Synthesis of 1,3-dimethyl 2,2-dimethyl-5-(tetramethyl-1,3,2-dioxaborolan-2-yl)cyclohex-4-ene-1,3-dicarboxylate

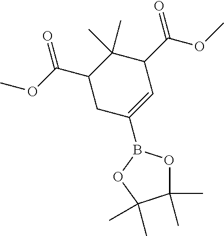

Into a 250 ml round-bottom flask purged and maintained with an inert atmosphere of nitrogen, was placed 1,3-dimethyl 2,2-dimethyl-5-[(trifluoromethane)sulfonyloxy]cyclohex-4-ene-1,3-dicarboxylate (2.1 g, 5.61 mmol, 1.00 equiv.), 4,4,5,5-tetramethyl-2-(tetramethyl-1,3,2-dioxaborolan-2-yl)-1,3,2-dioxaborolane (1.71 g, 6.73 mmol, 1.20 equiv.), PdCl₂.dppf (410 mg), KOAc (1.65 g, 16.81 mmol, 3.00 equiv.), 1,4-dioxane (40 ml). The resulting solution was stirred for 16 h at 100° C. The resulting mixture was concentrated under vacuum. The residue was applied onto a silica gel column with ethyl acetate/petroleum ether (1:5). This resulted in 1.2 g of the title compound as a light yellow solid (61%). LCMS: 1.12 min, m/z=353.2 [M+1]. ¹H-NMR (300 MHz, CDCl₃) δ: 6.39 (s, 1H), 3.70 (s, 3H), 3.65 (s, 3H), 3.06 (brs, 1H), 2.48-2.38 (m, 3H), 1.25 (s, 12H), 1.02 (s, 3H), 0.99 (s, 3H).

### Step-6: Synthesis of dimethyl 5-(4-(((2-((tert-butoxycarbonyl)(methyl)amino)ethyl) (methyl)amino)methyl)-1-(tetrahydro-2H-pyran-2-yl)-1H-pyrazol-3-yl)-2,2-dimethylcyclohex-4-ene-1,3-dicarboxylate

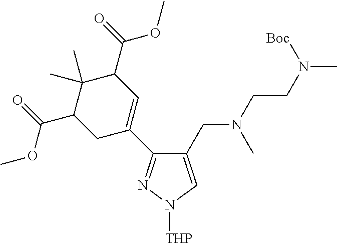

Into a 250 ml round-bottom flask purged and maintained with an inert atmosphere of nitrogen, was placed 1,3-dimethyl 2,2-dimethyl-5-(tetramethyl-1,3,2-dioxaborolan-2-yl)cyclohex-4-ene-1,3-dicarboxylate (1.2 g, 3.41 mmol, 1.00 equiv.), tert-butyl N-[2-([[3-iodo-1-(oxan-2-yl)-1H-pyrazol-4-yl]methyl](methyl)amino)ethyl]-N-methylcarbamate (1.48 g, 3.09 mmol, 0.91 equiv.), Pd(dppf)Cl₂ (227 mg, 0.31 mmol, 0.09 equiv.), 1,4-dioxane (30 ml), potassium carbonate (1.28 g, 9.26 mmol, 2.72 equiv.) and water (3 ml). The resulting solution was stirred for 20 h at 100° C. The resulting solution was diluted with 30 ml of water. The resulting solution was extracted with 3×30 ml of ethyl acetate and the organic layers combined. The resulting mixture was washed with 2×30 ml of NH₄Cl (sat. aq.). The resulting mixture was concentrated under vacuum. The residue was applied onto a silica gel column with ethyl acetate/petroleum ether (4:1). This resulted in 650 mg of the title compound as light brown oil (33%). LCMS: 1.63 min, m/z=577.1 [M+1].

### Step-7: Synthesis of sodium 3-(4-(((2-((tert-butoxycarbonyl)(methyl)amino)ethyl)(methyl)amino)methyl)-1-(tetrahydro-2H-pyran-2-yl)-1H-pyrazol-3-yl)-5-(methoxycarbonyl)-6,6-dimethylcyclohex-2-enecarboxylate

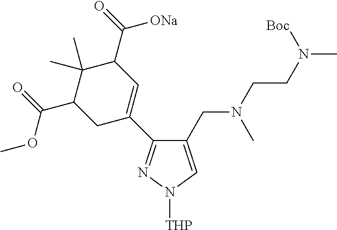

Into a 100 ml round-bottom flask, was placed 1,3-dimethyl 5-(4-[[(2-[[(tert-butoxy)carbonyl] (methyl)amino] ethyl)(methyl)amino]methyl]-1-(oxan-2-yl)-1H-pyrazol-3-yl)-2,2-dimethylcyclohex-4-ene-1,3-dicarboxylate (500 mg, 0.87 mmol, 1.00 equiv.), sodium hydroxide (696 mg, 17.40 mmol, 20.07 equiv.), water (1.5 g) and methanol (40 ml). The resulting solution was stirred for 16 h at 80° C. The resulting mixture was concentrated under vacuum. This resulted in 1.2 g (crude) of the title compound as a brown solid. LCMS: 1.23 min, m/z=563.2 [M-Na+1].

### Step-8: Synthesis of methyl 3-(4-(((2-((tert-butoxycarbonyl)(methyl)amino)ethyl)(methyl)amino)methyl)-1-(tetrahydro-2H-pyran-2-yl)-1H-pyrazol-3-yl)-5-(ethyl(methyl)carbamoyl)-6,6-dimethylcyclohex-3-enecarboxylate

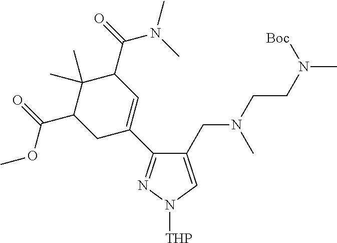

Into a 100 ml round-bottom flask, was placed 3-methyl 1-sodium 5-(4-[[(2-[[(tert-butoxy)carbonyl](methyl)amino]ethyl)(methyl)amino]methyl]-1-(oxan-2-yl)-1H-pyrazol-3-yl)-2,2-dimethylcyclohex-4-ene-1,3-dicarboxylate (350 mg, 0.60 mmol, 1.00 equiv.), ethyl(methyl)amine (146 mg, 2.47 mmol, 4.13 equiv.), HATU (471 mg, 1.95 mmol, 3.26 equiv.), dichloromethane (30 ml). And TEA (188 mg, 1.86 mmol, 3.10 equiv.) was added into mixture. The resulting solution was stirred for 16 h at room temperature. The resulting solution was diluted with 20 ml of water. The resulting mixture was washed with 3×40 ml of brine (sat.). The resulting mixture was concentrated under vacuum. The residue was applied onto a silica gel column with ethyl acetate/petroleum ether (4:1). This resulted in 100 mg of the title compound as light yellow oil (28%). LCMS: 1.32 min, m/z=604.3 [M+1].

### Step-9: Synthesis of 3-(4-(((2-((tert-butoxycarbonyl)(methyl)amino)ethyl)(methyl)amino)methyl)-1-(tetrahydro-2H-pyran-2-yl)-1H-pyrazol-3-yl)-5-(ethyl(methyl)carbamoyl)-6,6-dimethylcyclohex-3-enecarboxylic acid

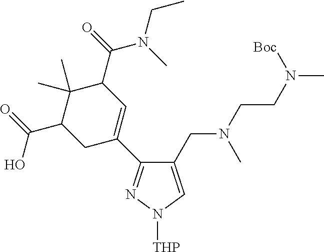

Into a 100 ml round-bottom flask, was placed methyl 3-(4-[[(2-[[(tert-butoxy)carbonyl](methyl) amino]ethyl)(methyl)amino]methyl]-1-(oxan-2-yl)-1H-pyrazol-3-yl)-5-[ethyl(methyl)carbamoyl]-6,6-dimethylcyclohex-3-ene-1-carboxylate (100 mg, 0.17 mmol, 1.00 equiv.), potassium hydroxide (250 mg, 4.46 mmol, 26.90 equiv.), water (1 ml), methanol (10 ml). The resulting solution was stirred for 2 d at 75° C. The resulting mixture was concentrated under vacuum. This resulted in 350 mg of the title compound as a light yellow solid that was used in the next step without further purification. LCMS: 1.23 min, m/z=590.3 [M+1].

### Step-10: Synthesis of tert-butyl (2-(((3-(3-(ethyl(methyl)carbamoyl)-5-(isopentylcarbamoyl)-4,4-dimethylcyclohex-1-en-1-yl)-1-(tetrahydro-2H-pyran-2-yl)-1H-pyrazol-4-yl)methyl)(methyl)amino)ethyl)(methyl)carbamate

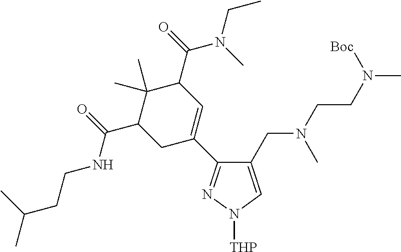

Into a 100 ml round-bottom flask, was placed 3-(4-[[(2-[[(tert-butoxy)carbonyl](methyl) amino]ethyl)(methyl)amino]methyl]-1-(oxan-2-yl)-1H-pyrazol-3-yl)-5-[ethyl(methyl)carbamoyl]-6,6-dimethylcyclohex-3-ene-1-carboxylic acid (350 mg, crude, 1.00 equiv.), 3-methylbutan-1-amine (59 mg, 0.68 mmol, 3.99 equiv.), TEA (52 mg, 0.51 mmol, 3.03 equiv.), dichloromethane (20 ml), HATU (129 mg, 0.54 mmol, 3.16 equiv.). The resulting solution was stirred for 16 h at room temperature. The resulting solution was diluted with 20 ml of water. The resulting mixture was washed with 2×20 ml of brine (sat.). The resulting mixture was concentrated under vacuum. The residue was applied onto a silica gel column with DCM/MeOH (1:10). This resulted in 45 mg of the title compound as light yellow oil (40%). LCMS: 0.87 min, m/z=659.4 [M+1].

### Step-11: Synthesis of tert-butyl (2-(((3-(3-(ethyl(methyl)carbamoyl)-5-(isopentylcarbamoyl)-4,4-dimethylcyclohexyl)-1-(tetrahydro-2H-pyran-2-yl)-1H-pyrazol-4-yl)methyl)(methyl)amino)ethyl)(methyl)carbamate

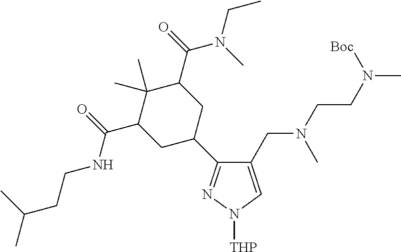

Into a 50 mL round-bottom flask, was placed tert-butyl N-(2-[[(3-[3-[ethyl(methyl)carbamoyl]-4,4-dimethyl-5-[(3-methylbutyl)carbamoyl]cyclohex-1-en-1-yl]-1-(oxan-2-yl)-1H-pyrazol-4-yl)methyl](methyl)amino]ethyl)-N-methylcarbamate (60 mg, 0.09 mmol, 1.00 equiv.), oxolane (15 ml). Then 10% of Pd(OH)₂/C (120 mg) was added. Then hydrogen was introduced into mixture and maintained at 2 atm pressure. The resulting solution was stirred for 16 h at room temperature. The solids were filtered out. The resulting mixture was concentrated under vacuum. The crude product (60 mg) was purified by Prep-HPLC with the following conditions: Column, Xbridge Prep Phenyl, 5 um, 19×150 mm; mobile phase, Water with 50 mmol ammonium bicarbonate and acetonitrile (10.0% acetonitrile up to 33.0% in 2 min, up to 53.0% in 8 min, up to 100.0% in 1 min, down to 10.0% in 1 min); Detector, UV 220 nm. 10 mg product was obtained. This resulted in 10 mg of the title compound as light yellow oil (17%). LCMS: 0.87 min, m/z=661.4 [M+1].

### Step-12: Synthesis of N1-ethyl-N3-isopentyl-N1,2,2-trimethyl-5-(4-((methyl(2-(methylamino)ethyl)amino)methyl)-1H-pyrazol-3-yl)cyclohexane-1,3-dicarboxamide bis(trifluoroacetic acid)

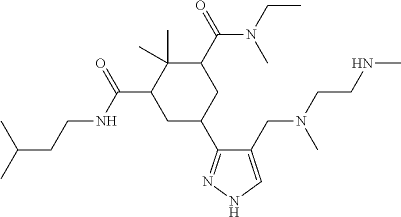 [SMILES: CCN(C)C(=O)C1CC(c2n[nH]cc2CN(C)CCNC)CC(C(=O)NCCC(C)C)C1(C)C]

Into a 25 ml round-bottom flask, was placed tert-butyl N-(2-[[(3-[3-[ethyl(methyl)carbamoyl]-4,4-dimethyl-5-[(3-methylbutyl)carbamoyl]cyclohexyl]-1-(oxan-2-yl)-1H-pyrazol-4-yl)methyl] (methyl)amino]ethyl)-N-methylcarbamate (10 mg, 0.02 mmol, 1.00 equiv.), dichloromethane (2 ml), trifluoroacetic acid (1 ml). The resulting solution was stirred for 0.5 h at room temperature. The resulting mixture was concentrated under vacuum. This resulted in 8.4 mg of the title compound as a light yellow solid (79%). LCMS: 1.31 min, m/z=477.3 [M+1]. ¹H-NMR (400 MHz, D₂O) δ: 7.73 (d, J=2.4 Hz, 1H), 4.35 (d, J=2.8 Hz, 2H), 3.82 (t, J=8.4 Hz, 1H), 3.75-3.35 (m, 5H), 3.25-3.12 (m, 3H), 3.11-3.00 (m, 2H), 2.84-2.81 (m, 5H), 2.70 (s, 3H), 2.20 (d, J=4.8 Hz, 1H), 1.98-1.62 (m, 3H), 1.60-1.42 (m, 2H), 1.32 (q, J=6.8 Hz, 2H), 1.12-1.04 (m, 4H), 0.98 (t, J=6.8 Hz, 2H), 0.91 (d, J=3.6 Hz, 3H), 0.79 (dd, J=6.8 and 1.6 Hz, 6H). F-NMR (400 MHz, D₂O) δ: −75.61.

### Example B-13. Synthesis of N1-((3-((5r,8r)-3,3-dimethyl-2-oxaspiro[4.5]decan-8-yl)-1H-pyrazol-4-yl)methyl)-N1,N2-dimethylethane-1,2-diamine (Compound 126)

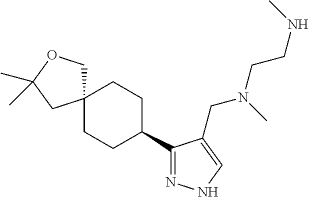 [SMILES: CNCCN(C)Cc1c[nH]nc1[C@H]1CC[C@@]2(CC1)COC(C)(C)C2]

### Step 1: Synthesis of 11,11-dimethyl-1,4,10-trioxadispiro[4.2.48.25]tetradecan-9-one

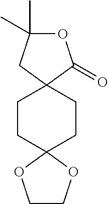 [SMILES: CC1(C)CC2(CCC3(CC2)OCCO3)C(=O)O1]

2M lithium dipropan-2-ylazanide in hexane (18.7 ml, 37.3 mmol) was added dropwise over 30 minutes to a −78° C. solution of ethyl 1,4-dioxaspiro[4.5]decane-8-carboxylate (4 g, 18.7 mmol) and 2,2-dimethyloxirane (8.3 mL, 93 mmol) in anhydrous THF (40 ml). The reaction was stirred at −78° C. for a further 2 hours, allowing to warm to 20° C. over 15 h. The reaction was quenched by addition of NH₄Cl (saturated, aqueous 40 ml) and extracted with DCM (3×40 ml). The combined organics were washed with brine (40 ml) and dried over magnesium sulfate, filtered and concentrated under vacuum. Upon returning to atmospheric pressure the yellow oil solidified/crystallised and was sonicated in warm heptane (30 ml) and filtered to yield 2.7 g of the title compound as a white solid (60%). LC/MS 1.09 min; m/z=241.4 [M+1]; ¹H NMR (250 MHz, CDCl₃-d) δ: 3.95 (s, 4H), 2.18-2.05 (m, 2H), 2.05 (s, 2H), 1.89 (dt, J=13.4, 3.8 Hz, 2H), 1.73-1.62 (m, 2H), 1.62-1.53 (m, 1H), 1.52-1.47 (m, 1H), 1.45 (s, 6H).

### Step-2: Synthesis of 1-[8-(hydroxymethyl)-1,4-dioxaspiro[4.5]decan-8-yl]-2-methylpropan-2-ol

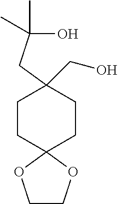 [SMILES: CC(C)(O)CC1(CO)CCC2(CC1)OCCO2]

To a stirred solution of 4M LiAlH4 in Et₂O (3.39 ml) in Et₂O (40 ml) was added drop wise a solution of 11,11-dimethyl-1,4,10-trioxadispiro[4.2.48.25]tetradecan-9-one (3.26 g, 13.55 mmol) in THF (20 ml) at 20° C., and the reaction stirred for 17 h. The reaction was cooled to 0° C. and water (514 μl), NaOH (15% aqueous, 514 μl), and then water (1.54 ml) were slowly added sequentially, dropwise. The mixture was allowed to warm to room temp and stirred for 30 min. MgSO₄ was added and the mixture filtered through Celite, washing with EtOAc. The filtrate was concentrated to yield 3.28 g of the title compound as a white solid (99%). LCMS: 1.44 min, m/z=267.05 [M+Na], 308.15 [M+Na+MeCN]. ¹H NMR (250 MHz, CDCl₃) δ: 3.92 (s, 4H), 3.59 (s, 2H), 1.72-1.47 (m, 10H), 1.31 (s, 6H).

### Step-3: Synthesis of 3,3-dimethyl-2-oxaspiro[4.5]decan-8-one

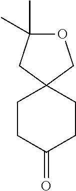

1-[8-(hydroxymethyl)-1,4-dioxaspiro[4.5]decan-8-yl]-2-methylpropan-2-ol (3.28 g, 13.44 mmol) was stirred in 4M hydrogen chloride in dioxane (30 ml) for 3 hours (TLC indicated SM consumed). 2M hydrogen chloride in Water (15 ml) was added and the reaction stirred for 18 hours. The reaction mixture was extracted with EtOAc (3×30 ml) to yield 3.3 g crude product (1H NMR shows 25% acetal intermediate remained). Crude product was stirred in THF (15 ml) and 2M hydrogen chloride in Water (15 ml) for 9 hours. The mixture was extracted with EtOAc (3×30 ml) to yield 3.05 g (20% SM remained). The aqueous layers were concentrated under vacuum, combined with the crude product, dissolved in DCM, dried over magnesium sulfate, and loaded onto a Biotage SNAP KP-Sil 25 g cartridge, eluting with 0-50% EtOAc in Heptane over 13 CV, to yield 1.42 g of the title compound as a golden oil (53%). ¹H NMR (250 MHz, CDCl₃) δ: 3.77 (s, 2H), 2.40-2.30 (m, 4H), 1.91 (t, J=6.8 Hz, 4H), 1.78 (s, 2H), 1.63 (s, 2H), 1.31 (s, 6H), 1.25 (s, 2H).

### Step-4: Synthesis of 3,3-dimethyl-1-oxo-2-oxaspiro[4.5]dec-7-en-8-yl trifluoromethanesulfonate

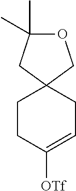 [SMILES: CC1(C)CC2(CC=C(OS(=O)(=O)C(F)(F)F)CC2)CO1]

1M LHMDS in THF (16 ml) was added slowly to a stirring solution of 3,3-dimethyl-2-oxaspiro[4.5]decan-8-one (1.42 g, 7.79 mmol) in THF (35 ml) at −70° C. The reaction was stirred at −70° C. for 90 min, then a solution of N-(5-chloropyridin-2-yl)-1,1,1-trifluoro-N-[(trifluoromethyl) sulfonyl]methanesulfonamide (5.5 g, 14 mmol) in THF (35 ml) was added slowly. The reaction was allowed to warm to 20° C. and stirred for 16 h for 18 h. (TLC 50% EtOAc in heptane showed no SM. A higher running compound was observed with DNP stain). The reaction was quenched with NH₄Cl (sat., 50 ml) and the solution was extracted into EtOAc (3×20 ml), the organics dried over magnesium sulfate, filtered and concentrated under vacuum. The residue was sonicated in heptane, concentrated under vacuum, sonicated in DCM and a white solid (impurity) removed by filtration. The filtrate was concentrated and loaded onto a 25 g Biotage SNAP KP-Sil cartridge and purified by FFC, eluting with 0-20% EtOAc in heptanes. Relevant fractions combined and concentrated under vacuum to yield 1.3 g of the title compound (50%). ¹H NMR (500 MHz, CDCl₃) δ: 5.72 (dt, J=4.1, 2.7 Hz, 1H), 3.63 (d, J=1.5 Hz, 2H), 2.38 (ddq, J=6.4, 4.0, 2.0 Hz, 2H), 2.34-2.14 (m, 2H), 1.88-1.72 (m, 2H), 1.66 (q, J=12.9 Hz, 2H), 1.29 (d, J=5.0 Hz, 6H).

### Step-5: Synthesis of 2-{3,3-dimethyl-2-oxaspiro[4.5]dec-7-en-8-yl}-4,4,5,5-tetramethyl-1,3,2-dioxaborolane

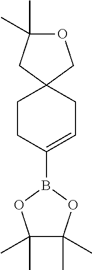

A solution of 3,3-dimethyl-2-oxaspiro[4.5]dec-7-en-8-yl trifluoromethanesulfonate (880 mg, 2.79 mmol), bis(pinacolato)diboron (730 mg, 2.87 mmol) and potassium acetate (770 mg, 7.85 mmol) in dioxane (15 ml) was degassed under a stream of nitrogen for 15 min, then PdCl₂(dppf)-DCM (120 mg, 0.15 mmol) was added. The solution was degassed under a stream of nitrogen for a further 10 min, then heated to 80° C. for 16 h. ¹H NMR showed no SM (1H at 5.7 ppm) and presence of product (1H at 6.4 ppm). The reaction mixture was filtered through Celite, washing with EtOAc until eluent colourless. The filtrate was concentrated azeotroping with heptane. The residues were slurried in MeCN (1 ml), and sonicated, then extracted (via sonication/vigorous stirring) into Heptane (5×10 ml). The heptane layers were combined and concentrated to yield 972 mg of the title compound as a brown viscous oil (85% purity, quantitative yield). LCMS: 1.55 min, m/z=292.95 [M+1]; ¹H NMR (250 MHz, CDCl₃) δ: 6.50 (s, 1H), 3.58 (s, 2H), 2.16 (dq, J=7.4, 3.5, 3.1 Hz, 4H), 1.65-1.53 (m, 4H), 1.27 (s, 6H), 1.26 (s, 12H).

### Step-6: Synthesis of tert-butyl N-(2-{[(3-{3,3-dimethyl-2-oxaspiro[4.5]dec-7-en-8-yl}-1-(oxan-2-yl)-1H-pyrazol-4-yl)methyl](methyl)amino}ethyl)-N-methylcarbamate

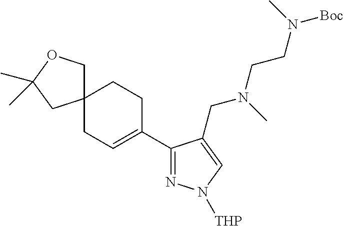

A solution of 2-{3,3-dimethyl-2-oxaspiro[4.5]dec-7-en-8-yl}-4,4,5,5-tetramethyl-1,3,2-dioxaborolane (230 mg, 0.79 mmol), N-[2-({[3-iodo-1-(oxan-2-yl)-1H-pyrazol-4-yl]methyl}(methyl)amino)ethyl]-N-methylcarbamate (250 mg, 0.52 mmol) in dioxane (10 ml) was degassed with N₂ for 10 min. K₂CO₃ (326 mg, 2.36 mmol) in water (1 ml) was degassed with N₂ for 10 min, then added to the reaction mixture. PdCl₂(dppf)-DCM (64 mg, 0.08 mmol) was added, the mixture degassed for a further 10 min, then heated to 80° C. for 22 h. tert-butyl N-[2-({[3-iodo-1-(oxan-2-yl)-1H-pyrazol-4-yl]methyl}(methyl)amino)ethyl]-N-methylcarbamate (50 mg, 0.1 mmol) was added and heated at 90° C. for a further 2 h. The reaction was concentrated under vacuum, and dry loaded on silica, to a Biotage SNAP KP-Sil 10 g cartridge, eluting with 0-100% EtOAc in Heptane over 3CV, then 0-20% MeOH in EtOAc over 14CV. All fractions containing product were combined and concentrated onto Silica, and loaded onto a Biotage SNAP HP 10 g cartridge, eluting with 0-25% EtOAc in heptane over 1CV, 25-100% EtOAc in Heptane over 6CV, and 0-10% MeOH in EtOAc over 4CV, to yield 350 mg the title compound (33%). LCMS: 1.11 min, m/z=517.7 [M+1]. ¹H NMR (250 MHz, CDCl₃) δ: 6.15 (s, 1H), 4.26-4.02 (m, 2H), 3.68 (s, 2H), 3.38 (d, J=6.9 Hz, 4H), 2.85 (d, J=6.2 Hz, 3H), 2.52 (d, J=6.5 Hz, 4H), 2.26 (d, J=8.2 Hz, 3H), 2.07 (s, 4H), 1.82-1.56 (m, 8H), 1.46 (s, 9H), 1.35-1.26 (m, 6H).

### Step-7: Synthesis of tert-butyl N-(2-{[(3-{3,3-dimethyl-2-oxaspiro[4.5]decan-8-yl}-1-(oxan-2-yl)-1H-pyrazol-4-yl)methyl](methyl)amino}ethyl)-N-methylcarbamate

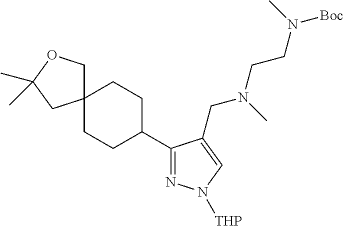

A solution of tert-butyl N-(2-{[(3-{3,3-dimethyl-2-oxaspiro[4.5]dec-7-en-8-yl}-1-(oxan-2-yl)-1H-pyrazol-4-yl)methyl](methyl)amino}ethyl)-N-methylcarbamate (77%, 350 mg, 0.52 mmol) in EtOH (20 ml) was cautiously added onto a 50% suspension of Raney Ni in water (2 ml, 11.27 mmol) in EtOH (20 ml). The resulting solution was purged with Nitrogen (3×), Hydrogen (3×) and left under an atmosphere of hydrogen at −20° C. for 21 h. The reaction was purged with Nitrogen (3×), and reaction mixture was filtered through Celite washing with EtOH (4 ml). Fresh Raney Ni (1 ml) was added to the filtrate and reaction was stirred under Nitrogen for 3 days, then under Hydrogen for 7 h. LCMS showed mainly starting material present. The hydrogen was removed, and reaction mixture was filtered through Celite washing with EtOAc (20 ml). To a solution of recovered tert-butyl N-(2-{[(3-{3,3-dimethyl-2-oxaspiro[4.5]dec-7-en-8-yl}-1-(oxan-2-yl)-1H-pyrazol-4-yl)methyl](methyl)amino}ethyl)-N-methylcarbamate (77%, 255 mg, 0.38 mmol) in 30 mL EtOH was added wet Raney nickel suspension in water (5 ml). The resulting solution was purged with Nitrogen (3×), Hydrogen (3×) and left under an atmosphere of hydrogen at rt for 3 h after which time, LCMS showed complete reaction (1/1, cis/trans isomer mixture). The reaction mixture was carefully filtered through a short Celite pad and washed with MeOH (100 ml). The filtrate was evaporated to dryness. The residue was dissolved in EtOAc and filtered. The filtrate was evaporated to dryness and then purified by high pH prep (3 injections) to afford the cis isomer as a colorless glass (40%) and the trans isomer as a colorless glass (30%).

Trans: ¹H NMR (500 MHz, CDCl₃) δ: 7.40 (s, 1H), 5.33-5.16 (m, 1H), 4.04 (d, J=11.1 Hz, 1H), 3.74 (s, 2H), 3.66 (t, J=11.4 Hz, 1H), 3.29 (d, J=27.3 Hz, 4H), 2.81 (s, 4H), 2.59 (t, J=11.9 Hz, 1H), 2.43 (s, 2H), 2.18 (s, 3H), 2.04-1.92 (m, 3H), 1.81 (t, J=14.4 Hz, 4H), 1.69-1.51 (m, 7H), 1.43 (d, J=6.7 Hz, 11H), 1.25 (s, 6H).

### Step-8: Synthesis of N1-((3-((5r,8r)-3,3-dimethyl-2-oxaspiro[4.5]decan-8-yl)-1H-pyrazol-4-yl)methyl)-N1,N2-dimethylethane-1,2-diamine

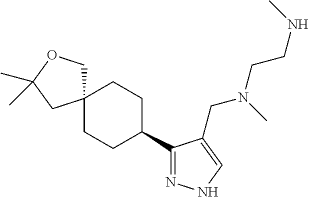 [SMILES: CNCCN(C)Cc1c[nH]nc1[C@H]1CC[C@@]2(CC1)COC(C)(C)C2]

The solution of tert-butyl N-(2-{[(3-{3,3-dimethyl-2-oxaspiro[4.5]decan-8-yl}-1-(oxan-2-yl)-1H-pyrazol-4-yl)methyl](methyl)amino}ethyl)-N-methylcarbamate (isomer 1, 79 mg, 0.15 mmol) in dioxane (5 mL) was added with 6N HCl in water (3 mL) and then stirred at room temperature for 24 h. The reaction mixture was evaporated to dryness. The residue was loaded on a SCX-2 (2 g) column, washed MeOH (20 mL), and then flushed with 7N NH₃ in MeOH (20 ml). The desired fractions were combined and evaporated to afford the trans isomer as colourless glass (100%).

Trans: LCMS: 2.11 min, m/z=335 [M+1]. ¹H NMR (500 MHz, CDCl₃) δ: 7.37 (s, 1H), 3.75 (s, 2H), 3.35 (s, 2H), 2.73-2.62 (m, 3H), 2.50 (t, J=6.0 Hz, 2H), 2.43 (s, 3H), 2.14 (s, 3H), 1.93-1.80 (m, 4H), 1.62 (s, 2H), 1.58-1.43 (m, 4H), 1.27 (s, 6H).

### Example B-14. Synthesis of (5s,8s)-3,3-diethyl-8-(4-((methyl(2-(methylamino)ethyl)amino)methyl)-1H-pyrazol-3-yl)-1-azaspiro[4.5]decan-2-one and (5r,8r)-3,3-diethyl-8-(4-((methyl(2-(methylamino)ethyl)amino)methyl)-1H-pyrazol-3-yl)-1-azaspiro[4.5]decan-2-one (Compound 47 and 48)

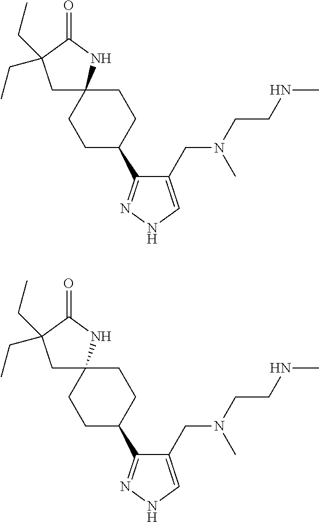

### Step-1: Synthesis of methyl 3-(4-methoxyphenyl)propanoate

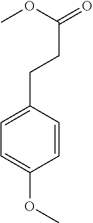

To a stirred solution of thionyl chloride (3.0 ml, 41.62 mmol) in MeOH (30 ml) at cooling with dry ice/methanol bath to rt was added 3-(4-methoxyphenyl)propanoic acid (5 g, 27.74 mmol) after stirring at rt for 1 h. The solvent was evaporated under reduced pressure, diluted with ethyl acetate and washed with aqueous saturated sodium bicarbonate and brine, then dried over sodium sulphate. The solvent was removed under reduced pressure and the resulting crude was purified by column chromatography, eluted at 2% ethyl acetate in hexane to get the title product (94.2%). LCMS: m/z=195.2 [M+1]. ¹H-NMR (DMSO, 400 MHz) δ: 7.14-7.12 (dd, 2H J=8.4 Hz), 6.84-6.82 (dd, J=8.4 Hz, 2H), 3.71 (s, 3H), 3.57 (s, 3H), 2.78 (t, 2H), 2.58-2.56 (t, 2H).

### Step-2: Synthesis of methyl 2-ethyl-2(4-methoxybenzyl)butanoate

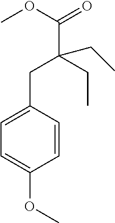 [SMILES: CCC(CC)(Cc1ccc(OC)cc1)C(=O)OC]

To a stirred solution of the diisopropyl amine (9.69 g 95.7 mmol) in dry THF (60 ml) under nitrogen atmosphere cooled at −78° C. was added n-BuLi (58 ml, 92.64 mmol, 2M in Hexane) stirred reaction mixture 0° C. for 30 min. Methyl 3-(4-methoxyphenyl)propanoate (3 g, 15.44 mmol) in THF was added at −78° C. The reaction mixture was stirred at −78° C. for 30 min and added ethyl iodide (16.8 g, 108 mmol), stirred reaction mixture at −78° C. for 3.5 h. The reaction mixture was cooled to room temperature and quenched by ammonium chloride solution and extracted with ethyl acetate (3×30 ml), dried over sodium sulphate, concentrated under reduced pressure and the resulting crude material was purified by column chromatography to afford title compound (77.7%). LCMS: m/z=251.4 [M+1]. ¹H-NMR (DMSO, 400 MHz) δ: 7.02-7.00 (dd, J=8.8 Hz, 2H), 6.82-6.80 (dd, J=8.8 Hz, 2H), 3.63 (s, 3H), 3.57 (s, 3H), 2.83 (s, 2H), 1.66-1.50 (t, 4H), 0.88 (t, 6H).

### Step-3: Synthesis of methyl 2-ethyl 2-(4-methoxybenzyl)butanoic acid

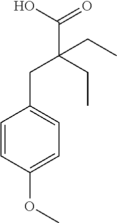 [SMILES: CCC(CC)(Cc1ccc(OC)cc1)C(=O)O]

To a stirred solution of methyl 2-ethyl-2(4-methoxybenzyl) butanoate (10 g, 40.0 mmol) in ethanol (160 ml) was added aq. KOH solution (8.9 g, 16.0 mmol, in 64 ml H₂O). After stirring at 90° C. for 48 h, The reaction mixture was cooled to rt and acidified using aq. 6N HCl and the mixture was extracted with ethyl acetate. The combined organic layers were dried over sodium sulphate and evaporated. The resulting crude was purified by column chromatography, eluted with 5% ethyl acetate in hexane to afford a yellowish solid product (85.1%). LCMS: m/z=235.5 [M−1]. ¹H-NMR (DMSO, 400 MHz) δ: 12.26 (s, 1H), 7.05-7.03 (dd, J=8.4 Hz, 2H), 6.83-6.81 (dd, J=8.4 Hz, 2H), 3.71 (s, 3H), 2.71 (s, 2H), 1.49-1.34 (m, 4H), 0.83-0.80 (t, 6H).

### Step-4: Synthesis of 2-ethyl-N-methoxy-2(-4-methoxybenzyl)butanamide

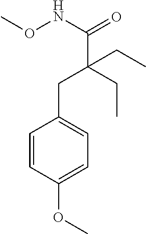 [SMILES: CCC(CC)(Cc1ccc(OC)cc1)C(=O)NOC]

To a solution of methyl 2-ethyl 2-(4-methoxybenzyl)butanoic acid (2 g 8.51 mmol) in DMF (20 ml) was added HATU (24.85 g, 12.7659 mmol) at 0° C. and stirred reaction mixture for 45 min at same temperature. O-methyl hydroxylamine hydrochloride (1.4 g, 17.02 mmol) and DIPEA (5.8 ml, 34.04 mmol) was added sequentially. The reaction mixture was stirred at rt for 4 h, diluted in water (20 ml) and extracted with ethyl acetate (3×20 ml), dried over sodium sulphate and concentrated under reduced pressure. The crude material was purified by column chromatography and the product was eluted with 30 EA in hexane. The pure fractions were evaporated to give title compound (88.8%). LCMS: m/z=266.5 [M+1]. ¹H-NMR (DMSO, 400 MHz) δ: 10.80 (s, 1H), 7.02-7.00 (dd, J=8.8 Hz, 2H), 6.82-6.80 (dd, J=8.8 Hz, 2H), 3.70 (s, 3H), 3.54 (s, 2H), 2.67 (s, 2H), 1.45-1.30 (m, 4H), 0.81-0.77 (t, 6H).

### Step-5: Synthesis of 3,3-diethyl-1-methoxy-1-azaspiro[4.5]deca-6,9-diene-2,8-dione

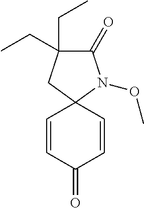 [SMILES: CCC1(CC)CC2(C=CC(=O)C=C2)N(OC)C1=O]

To a stirred solution of the above 2-ethyl-N-methoxy-2(-4-methoxybenzyl)butanamide (27 g 101.88 mmol) in MeOH (540 ml) at 0° C. temperature was added bis(trifluoroacetoxyiodo)benzene (65.71 g, 152.83 mmol) in dichloromethane. The reaction mixture was stirred for 5 min at which time H₂O (135 ml) was added and the resulting mixture was stirred for 10 min and quenched with saturated sodium bicarbonate solution and extracted in dichloromethane (3×200 ml). The combined extracts were dried over sodium sulphate and concentrated under reduced pressure. The crude material was purified by column chromatography to afford title compound (84%). LCMS: m/z=250.5 [M+1]. ¹H-NMR (DMSO, 400 MHz) δ: 7.09-7.05 (dd, J=8.8 Hz, 2H), 6.30-6.27 (dd, J=8.8 Hz, 2H), 2.50 (s, 3H), 1.57-1.52 (m, 4H), 0.89-0.85 (t, 6H).

### Step-6: Synthesis of 3,3-diethyl-1methoxy-1-azospiro[4.5]decane-2,8-dione

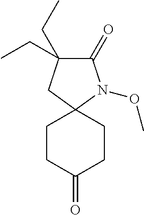 [SMILES: CCC1(CC)CC2(CCC(=O)CC2)N(OC)C1=O]

In a hydrogenation vessel charged with 10% Pd/C (9.6 g) and 3,3-diethyl-1-methoxy-1-azaspiro[4.5]deca-6,9-diene-2,8-dione (16 g, 64.17 mmol) in ethyl acetate (800 ml). The hydrogenation vessel was pressurized with H₂ up to 500 psi. The reaction mixture was stirred at rt for 5 h. The reaction mixture was filtered through Celite pad and washed with ethyl acetate, the resulting crude material was purified by column chromatography using mobile phase 0-1% methanol in dichloromethane to give the title compound (27.6%). LCMS: m/z=254.4 [M+1]. 1H-NMR (DMSO, 400 MHz) δ: 3.77 (s, 3H), 2.62-2.70 (m, 2H) 2.16-2.28 (m, 4H), 1.44-1.50 (m, 4H), 0.85-0.90 (m, 6H).

### Step-7: Synthesis of 3,3-diethyl-1-methoxyl-2-oxo-1-azaspiro.2.[4.5]dec-7-en-8-yl trifluoromethanesulfonate

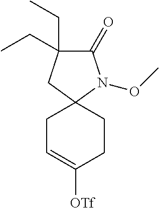 [SMILES: CCC1(CC)CC2(CC=C(OS(=O)(=O)C(F)(F)F)CC2)N(OC)C1=O]

To a stirred solution of 3,3-diethyl-1-methyl-1-azaspiro[4.5] decane-2,8-dione (2.6 g, 10.27 mmol) in DCM (40 ml) was added 2-(tert-butyl)-6-methylpyridine (4.7 g, 17.47 mmol) at rt. The reaction mixture was stirred for 30 min. The reaction was cooled to 0° C. and triflic anhydride (4.92 g, 17.47 mmol) was added and the reaction was stirred at rt for 16 h. The reaction mixture was diluted with sat. sodium bicarbonate solution (20 ml) and extracted with DCM (3×20 ml). Combined organic layers were dried over sodium sulphate. The solvent was removed under reduced pressure and the resulting crude was purified by column chromatography using mobile phase 0-25% ethyl acetate in hexane to give title compound (75.9%). LCMS: m/z=386.59 [M+1]. ¹H-NMR (DMSO, 400 MHz) δ: 5.86 (m, 1H), 3.76 (s, 3H), 2.69-2.52 (m, 2H), 2.11-2.17 (m, 2H), 1.60-1.85 (m, 6H), 1.40-1.50 (m, 4H), 0.88-0.85 (t, 6H).

### Step-8: Synthesis of 3,3-diethyl-1-methoxy-8-(4,4,5,5-tetramethyl-1,3,2-dioxaborolan-2-yl)-1-azaspiro[4.5] dec-7-en-2-one

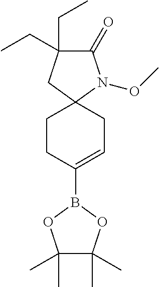 [SMILES: CCC1(CC)CC2(CC=C(B3OC(C)(C)C(C)(C)O3)CC2)N(OC)C1=O]

To the three neck RBF charged with 3, 3-diethyl-1-methoxyl-2-oxo-1-azaspiro.2.[4.5]dec-7-en-8-yl trifluoromethanesulfonate (3 g, 7.79 mmol), bispinacolato diborane (2.5 g, 9.35 mmol) and potassium acetate (3.05 g, 31.16 mmol) in 1,4-dioxane (40 ml) was purged with argon gas for 30 min. PdCl₂(dppf) (0.57 g, 0.779 mmol) was added and reaction mixture was heated at 100° C. for 1 h. The reaction mixture was filtered through Celite and washed with ethyl acetate (100 ml). The organic layer was washed with water and brine solution then dried over anhydrous sodium sulfate. The solvent was evaporated under reduced pressure. The crude compound was purified by column chromatography (stationary phase-60-120 silica gel). The desired compound was eluted in 40% ethyl acetate in n-hexane (98.9%). LCMS: m/z=364.74 [M+1]. ¹H-NMR (DMSO, 400 MHz) δ: 6.37-6.36 (t, 1H), 3.74 (s, 3H), 2.57-1.73 (m, 8H), 1.29-1.04 (s, 12H), 0.91-0.81 (t, 6H).

### Step-9: Synthesis of 3-(3,3-diethyl-1-methoxy-2-oxo-1-azaspiro[4.5] dec-7-en-8-yl)-1-(tetrahydro-2H-pyran-2-yl)-1H-pyrazol-4-carbaldehyde

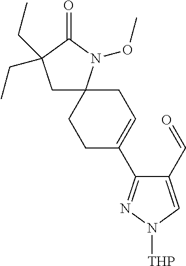

To a three neck RBF charged with 3,3-diethyl-1-methoxy-8-(4,4,5,5-tetramethyl-1,3,2-dioxaborolan-2-yl)-1-azaspiro[4.5] dec-7-en-2-one (1.6 g, 5.22 mmol),3-iodo-1-(tetrahydro-2H-pyran-2-yl)-1H-pyrazole-4-carbaldehyde (2.84 g, 7.84 mmol), potassium carbonate (3.325 g, 15.68 mmol) in 1,4-dioxane (20 ml) and water (8 ml) mixture; purged with argon gas for 30 minutes. PdCl₂(dppf) (0.382 g, 0.522 mmol) was added to reaction mixture and heated at 100° C. for 1 h. The reaction mixture was filtered through Celite and washed with ethyl acetate (50 ml), the filtrate was washed with brine and the combined organic layer was dried over anhydrous sodium sulfate followed by evaporation under reduced pressure. The crude material was purified by column chromatography using mobile phase 40% ethyl acetate in hexane to give the title compound (67.7%). LCMS: m/z=416.25 [M+1].

### Step-10: Synthesis of 3-(3,3-diethyl-1-methoxy-2-oxo-1-azaspiro[4.5]dec-7-en-8-yl)-1H-pyrrazole-4-carbaldehyde

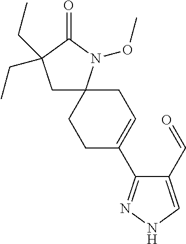 [SMILES: CCC1(CC)CC2(CC=C(c3n[nH]cc3C=O)CC2)N(OC)C1=O]

3-(3,3-diethyl-1-methoxy-2-oxo-1-azaspiro[4.5] dec-7-en-8-yl)-1-(tetrahydro-2H-pyran-2-yl)-1H-pyrazol-4-carbaldehyde (2.2 g) was added to a mixture of THF (20 ml), acetic acid (20 ml) and water (20 ml). The reaction mixture was heated at 80° C. for 5 h. The reaction mixture was neutralized by saturated sodium bicarbonate solution (50 ml) and extracted with ethyl acetate (3×50 ml). The combined organic layers were dried over anhydrous sodium sulphate. The solvent was removed under reduced pressure and the resulting crude was purified by column chromatography using mobile phase 2% MeOH in dichloromethane for elution of title compound (44.6%). LCMS: m/z=332.7[M+1].

### Step-11: Synthesis of tert-butyl(-(((3-(3,3-diethyl-1-methoxy-2-oxo-1-azaspiro[4.5]decan-8-yl)-1-1H-pyrazol-4-yl)(methyl)amino)ethyl)(methyl)carbamate

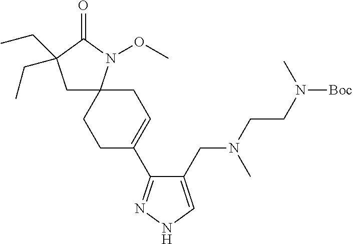 [SMILES: CCC1(CC)CC2(CC=C(c3n[nH]cc3CN(C)CCN(C)C(=O)OC(C)(C)C)CC2)N(OC)C1=O]

To a stirred solution of 3-(3,3-diethyl-1-methoxy-2-oxo-1-azaspiro[4.5]dec-7-en-8-yl)-1H-pyrrazole-4-carbaldehyde (0.58 g, 1.75 mmol) and tert-butyl(2-methylamino)ethyl)carbamate (0.495 g, 2.62 mmol) in EDC (25 ml) was added sodium triacetoxy borohydride (1.11 g, 5.26 mmol) portionwise at 0° C. The resulting mixture was stirred at rt for 2 h at which time the reaction mixture was diluted with water (30 ml) and extracted with ethyl acetate (2×20 ml) and the combined organic layers were dried over sodium sulphate. The solvent was removed under reduced pressure and the resulting crude was purified by column chromatography to give title compound (95.1%) LCMS: m/z=504.4 [M+1].

### Step-12: Synthesis of tert-butyl(-(((3-(3,3-diethyl-2-oxo-1-azaspiro[4.5]dec-7-en-8-yl)-1-1H-pyrazol-4-yl)(methyl)methyl)amino)ethyl)(methyl)carbamate

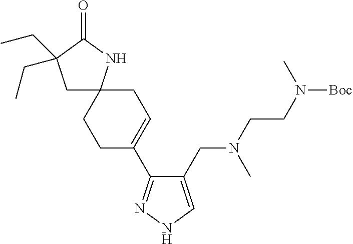 [SMILES: CCC1(CC)CC2(CC=C(c3n[nH]cc3CN(C)CCN(C)C(=O)OC(C)(C)C)CC2)NC1=O]

Tert-butyl(-(((3-(3,3-diethyl-1-methoxy-2-oxo-1-azaspiro[4.5]decan-8-yl)-1-1H-pyrazol-4-yl)(methyl)amino)ethyl)(methy)carbamate (0.99 g, 1.968 mmol) in THF (50 ml) was added slowly to a solution of sodium metal (0.104 g, 4.526 mmol) in freshly distilled ammonia (50 ml) cooled to −78° C. in a flask fitted with a dry ice condenser. The resulting mixture was stirred at same temperature for 30 min, then quenched with solid ammonium chloride (0.263 g, 4.92 mmol) and allowed to warm to rt over 3 h and filtered. The resulting filtrate was washed with ethyl acetate (3×20 ml) and then poured in water and extracted in ethyl acetate. The combined organic layer was removed under reduced pressure and the resulting crude material was purified by column chromatography (60-120 silica) using mobile phase 0-1% MeOH in DCM (63.6%). LCMS: m/z=474.4 [M+1].

### Step-13: tert-butyl (2-(((3-((5r,8r)-3,3-diethyl-2-oxo-1-azaspiro[4.5]decan-8-yl)-1H-pyrazol-4-yl)methyl)(methyl)amino)ethyl)(methyl)carbamate and tert-butyl (2-(((3-((5s,8s)-3,3-diethyl-2-oxo-1-azaspiro[4.5]decan-8-yl)-1H-pyrazol-4-yl)methyl)(methyl)amino)ethyl)(methyl)carbamate

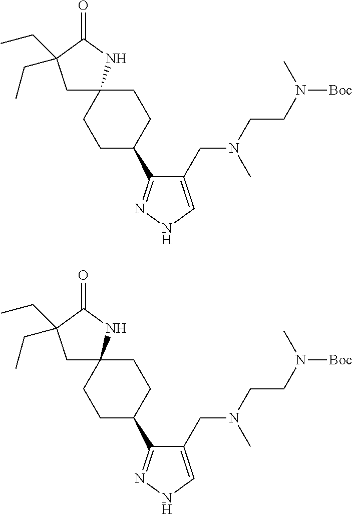 [SMILES: CCC1(CC)C[C@]2(CC[C@@H](c3n[nH]cc3CN(C)CCN(C)C(=O)OC(C)(C)C)CC2)NC1=O ; CCC1(CC)C[C@]2(CC[C@H](c3n[nH]cc3CN(C)CCN(C)C(=O)OC(C)(C)C)CC2)NC1=O]

A three-neck RBF was charged with 20% palladium hydroxide (0.3 g) followed by the addition of tert-butyl(-(((3-(3,3-diethyl-2-oxo-1-azaspiro[4.5]dec-7-en-8-yl)-1-1H-pyrazol-4-yl)(methyl)methyl)amino) ethyl)(methyl)carbamate (0.6 g, 1.262 mmol) in THF (40 ml) and MeOH (10 ml) were bubbled with H₂ at rt for 1 h. The reaction mixture was filtered through Celite pad and washed with 10% MeOH:DCM (50 ml). The filtrate was evaporated under reduced pressure to afford crude title compound. The diastereoisomers were separated by prep-HPLC using an X Bridge C18 column (250×19 mm) and 0.1% TFA in water, 100% acetonitrile as mobile phase to afford two fractions Fraction-I (220 mg) and Fraction-II (220 mg). LCMS: m/z=476.9 [M+1].

### Step-14: tert-butyl(2((3-(5s,8s)(3,3-diethyl-2-oxo-1-azaspiro[4.5]decan-8-yl)-1H-pyrazol-4-yl)(methyl)(methyl)amino)ethyl)(methyl)carbamate

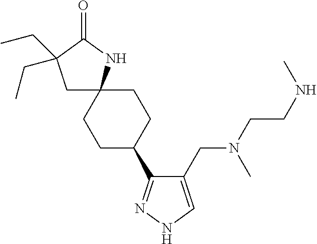 [SMILES: CCC1(CC)C[C@]2(CC[C@H](c3n[nH]cc3CN(C)CCNC)CC2)NC1=O]

Tert-butyl (2-(((3-((5s, 8s)-3,3-diethyl-2-oxo-1-azaspiro[4.5]decan-8-yl)-1H-pyrazol-4-yl)methyl)(methyl)amino)ethyl)(methyl)carbamate (0.220 g, 0.463 mmol) was added to a 4N IPA in HCl (5 ml) at rt, stirred for 24 h at rt. The solvent was removed under reduced pressure and triturated with n-pentane (20 ml) to give title compound (100 mg) as the HCl salt. LCMS: m/z=376.84 [M+1]. ¹H-NMR (D2O, 400 MHz) δ: 7.70 (s, 1H), 4.27 (s, 2H), 3.42 (m, 4H), 2.72-2.67 (m, 7H), 1.81 (s, 2H), 1.76-1.53 (m, 8H), 1.39-1.36 (q, 4H), 0.73 (t, 6H).

### Step-15: tert-butyl(2((3-(5r,8r)(3,3-diethyl-2-oxo-1-azaspiro[4.5]decan-8-yl)-1H-pyrazol-4-yl)(methyl)(methyl)amino)ethyl)(methyl)carbamate

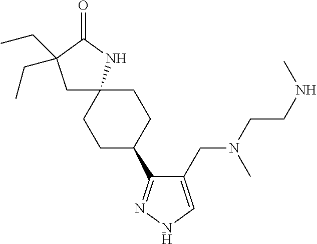 [SMILES: CCC1(CC)C[C@]2(CC[C@@H](c3n[nH]cc3CN(C)CCNC)CC2)NC1=O]

tert-butyl (2-(((3-((5r,8r)-3,3-diethyl-2-oxo-1-azaspiro[4.5]decan-8-yl)-1H-pyrazol-4-yl)methyl)(methyl)amino)ethyl)(methyl)carbamate (0.220 g, 0.463 mmol) was added to a 4N IPA in HCl (5 ml) at rt, stirred for 24 h at rt. The solvent was removed under reduced pressure and triturated with n-pentane (20 ml) to give title compound (110 mg) as HCl salt. LCMS: m/z=376.8 [M+1]. ¹H-NMR (D2O, 400 MHz) δ: 7.73 (s, 1H), 4.27 (s, 2H), 3.49 (m, 4H), 2.72-2.66 (m, 7H), 1.89 (s, 2H), 1.72-1.52 (m, 8H), 1.40-1.36 (q, 4H), 0.74 (t, 6H).

### Example B-15. Synthesis of 3,3-diethyl-1-methyl-8-(4-((methyl(2-(methylamino)ethyl)amino)methyl)-1H-pyrazol-3-yl)-1-azaspiro[4.5]decan-2-one (Compound 22)

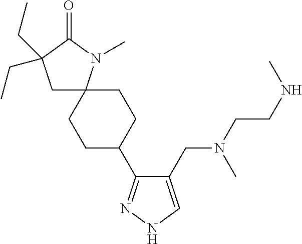 [SMILES: CCC1(CC)CC2(CCC(c3n[nH]cc3CN(C)CCNC)CC2)N(C)C1=O]

### Step-1: Synthesis of dimethyl 4-(3-methoxy-3-oxopropyl)-4-nitrohepanedioate

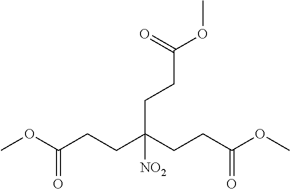

To a stirred solution of nitromethane (20.0 g, 328 mmol) in acetonitrile (400 ml) at 0° C. were added methyl acrylate (118 ml, 1.32 mol) and DBU (5.40 ml, 36.2 mmol). After stirring at 0° C. for 30 min, saturated aqueous ammonium chloride solution was added and the mixture was extracted with ethyl acetate (2×300 ml). The combined organic layer was washed with brine solution then dried over anhydrous sodium sulphate. The solvent was removed under reduced pressure and the resulting oil which was used in the next reaction without further purification (95.5%). ¹H-NMR (400 MHz, CDCl₃) δ: 3.70 (s, 9H), 2.36-2.26 (m, 12H).

### Step-2: Synthesis of dimethyl-3,3′-(5-oxopyrrolidine-2,2-diyldipropionate)

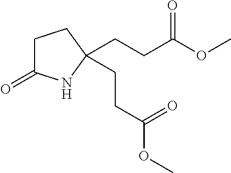 [SMILES: COC(=O)CCC1(CCC(=O)OC)CCC(=O)N1]

To a stirred solution of dimethyl 4-(3-methoxy-3-oxopropyl)-4-nitrohepanedioate (55.0 g 0.172 mmol) in MeOH (300 ml) at room temperature was added Raney-Nickel (5.5 g). After stirring at 60° C. under H₂ atm (5 kg/cm²) for 24 h, the mixture was filtered through a pad of Celite and washed with methanol. The solvent was removed under reduced pressure and the resulting crude product (40 g) was used in the next reaction without further purification. ¹H-NMR (400 MHz, CDCl₃) δ: 6.75 (s, 1H), 3.68 (s, 6H), 2.36 (m, 6H), 1.94-1.83 (m, 6H).

### Step-3: Synthesis of methyl 8-hydroxy-2-1-azaspiro(4,5)dec-7-ene-7-carboxylate

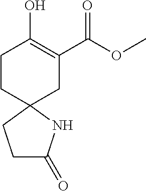 [SMILES: COC(=O)C1=C(O)CCC2(CCC(=O)N2)C1]

To a stirred solution of dimethyl 3, 3′-(5-oxopyrrolidine-2, 2-diyldipropionate) (40 g, 155.6 mmol) in benzene (300 ml) at 0° C. was added Methanol (0.6 ml, 15.56 mmol) followed by sodium hydride (60% in oil, 15.5 g, 389.10 mmol). After stirring at 90° C. for 30 min, aqueous 3N HCl was added and the mixture was extracted with ethyl acetate (3×200 ml). The combined organic layer was washed with saturated aqueous sodium bicarbonate solution and brine then dried over anhydrous sodium sulphate. The solvent was removed under reduced pressure. Resulting solid residue was triturated with hexane to afford the keto-ester title compound (62.8%) as a white solid. ¹H-NMR (400 MHz, CDCl₃) δ: 12.16 (s, 1H) 6.270 (br, 1H), 3.74 (s, 3H), 2.52-2.31 (m, 6H), 2.19-1.90 (m, 4H).

### Step-4: Synthesis of 1-azaspiro(4,5)dec-2,8-dione

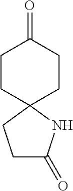 [SMILES: O=C1CCC2(CC1)CCC(=O)N2]

To a stirred solution of the above methyl 8-hydroxy-2-1-azaspiro (4, 5) dec-7-ene-7-carboxylate (22 g 97.77 mmol) in methanol (200 ml) at room temperature was added aqueous 4 N NaOH (7.82 g, 195.5 mmol). The reaction mixture was stirred at 100° C. for 3.5 h. After the reaction mixture was cooled to room temperature the mixture was carefully neutralized with acetic acid. The solvent was removed under reduced pressure and the resulting crude product was used in the next reaction without further purification. ¹H-NMR (400 MHz, CDCl₃) δ: 8.49 (brs, 1H), 2.63-2.39 (m, 6H), 1.94-2.11 (m, 6H).

### Step-5: Synthesis of 1,4-dioxa-9-azadispiro[4.2.4⁸.2⁵]tetradecan-10-one

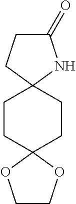 [SMILES: O=C1CCC2(CCC3(CC2)OCCO3)N1]

To a stirred solution of the 1-azaspiro-(4,5)-dec-2, 8-dione (8 g 47.9 mmol) in toluene (120 ml) at room temperature was added PTSA (0.91 g, 0.47 mmol) and ethylene glycol (9.3 ml, 167 mmol). The reaction mixture was stirred at 120° C. for 2.5 h in Dean-Stark assembly. After the reaction mixture was cooled to room temperature and extracted in ethyl acetate (3×150 ml), dried over sodium sulphate and concentrated under reduced pressure and the resulting crude purified by column chromatography to give title compound (84.1%). ¹H-NMR (400 MHz, CDCl₃) δ: 6.86 (s, 1H), 3.93 (s, 4H), 2.39-2.43 (q, 2H), 1.97 (t, 2H), 1.81-1.68 (m, 8H). LCMS: m/z=212 [M+1].

### Step-6: Synthesis of 9-methyl-1,4-dioxa-9-azadispiro(4.2.4⁸.2⁵)tetradecan-10-one

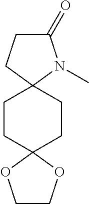 [SMILES: CN1C(=O)CCC12CCC1(CC2)OCCO1]

To a stirred solution of 1,4-dioxa-9-azaispiro[4.2.4⁸.2⁵]tetradecan-10-one (8.5 g 47.2 mmol) in THF (160 ml) was added sodium hydride (1.93 g, 48.3 mmol) at 0° C., stirred reaction mixture 10 min followed by addition of methyl iodide (12.59 g, 201 mmol). The reaction mixture was stirred at rt for 1 h. The reaction mixture was quenched by ice water and extracted with ethyl acetate (3×150 ml), dried over sodium sulphate, concentrated under reduced pressure. The crude material was purified by column chromatography to give title compound (91.6%). LCMS: m/z=226.2 [M+1]. ¹H-NMR (400 MHz, CDCl₃) δ: 3.97 (m, 4H), 2.76 (s, 3H), 2.42-2.37 (t, 2H), 2.07-1.95 (m, 4H), 1.77-1.70 (m, 4H), 1.44-1.40 (t, 2H).

### Step-7: Synthesis of 11,11-diethyl-9-methyl-1,4-dioxa-9-azadispiro(4.2.4⁸.2⁵)tetradecan-10-one

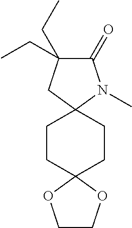 [SMILES: CCC1(CC)CC2(CCC3(CC2)OCCO3)N(C)C1=O]

To a stirred solution of diisopropylamine (16.73 g 27.5 mmol) in dry THF (120 ml) under argon atmosphere at 0° C. was added n-butyl lithium (180 ml, 159 mmol, 2M in hexane), and the reaction was cooled to −78° C. followed by the addition of 9-methyl-1,4-dioxa-9-azadispiro(4.2.4⁸.2)tetradecan-10-one (6 g, 26.6 mmol). The reaction mixture was stirred at −78° C. for 10 min, then ethyl iodide (15 ml, 186.66 mmol) was added and the reaction was stirred at −78° C. for 1 h. The reaction mixture was cooled to room temperature and quenched by ammonium chloride solution and extracted with ethyl acetate (3×100 ml), dried over sodium sulphate, concentrated under reduced pressure. The resulting crude material was purified by column chromatography using mobile phase 100% dichloromethane to give title compound (92.1%). ¹H-NMR (400 MHz, CDCl₃) δ: 3.95 (s, 4H), 2.76 (s, 3H), 2.11-2.01 (s, 2H), 1.82 (s, 2H), 1.80-1.74 (m, 4H), 1.59-1.49 (m, 4H), 1.38-1.34 (m, 2H), 0.89 (t, 6H).

### Step-8: Synthesis of 3,3-diethyl-1-methyl-1-azaspiro[4.5] decane-2,8-dione

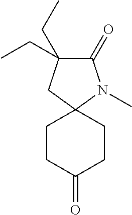 [SMILES: CCC1(CC)CC2(CCC(=O)CC2)N(C)C1=O]

To a stirred solution of 11,11-diethyl-9-methyl-1,4-dioxa-9-azadispiro[4.2.48.25]tetradecan-10-one (3.2 g, 0.014 mmol) in THF (30 ml) was added 3N HCl (20 ml) and the reaction mixture was stirred at rt for 3 h at which time the reaction was neutralized with aq. sodium bicarbonate. The compound was extracted with ethyl acetate (3×50 ml). The combined organic layers were washed with brine (10 ml) then dried over sodium sulphate. The solvent was removed under reduced pressure and the resulting oil was used in the next reaction without further purification. ¹H-NMR (400 MHz, CDCl₃) δ: 2.76 (s, 3H) 2.75-2.56 (m, 2H), 2.42-2.49 (m, 2H), 2.25-2.17 (m, 2H), 2.05 (s, 1H), 1.74-1.70 (m, 2H), 1.64-1.57 (m, 4H), 0.8-1.00 (t, 6H).

### Step-9: Synthesis of 3,3-diethyl-1-methyl-2-oxo-1-azaspiro[4.5]dec-7-en-8-yl trifluoromethanesulfonate

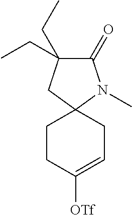 [SMILES: CCC1(CC)CC2(CC=C(OS(=O)(=O)C(F)(F)F)CC2)N(C)C1=O]

To a stirred solution of 3,3-diethyl-1-methyl-1-azaspiro[4.5] decane-2,8-dione (2.7 g, 11.5 mmol) in DCM (25 ml) at rt were added 2-(tert-butyl)-6-methylpyridine (4.7 g, 23.1 mmol) and triflic anhydride (9.81 g, 34.1 mmol). The reaction mixture was stirred for 30 min at rt, then heated at 50-60° C. for 1 h. The reaction mixture was diluted in 10% NaOH solution and extracted with DCM (3×20 ml). The combined organic layers were dried over sodium sulphate. The solvent was removed under reduced pressure and the resulting crude (3.2 g, yield 100%) was used in the next reaction. ¹H-NMR (400 MHz, CDCl₃) δ: 5.75 (s, 1H), 2.85 (s, 3H), 2.71-2.63 (m, 2H), 2.62-2.53 (m, 1H), 2.19-2.10 (s, 1H), 2.08-2.0 (m, 1H) 1.89-1.74 (m, 3H), 1.64-1.50 (q, 4H), 0.90-0.83 (t, 6H).

### Step-10: Synthesis of 3,3-diethyl-methyl-8-(4,4,5,5-tetramethyl-1,3,2-dioxaborolan-2-yl)-1-azaspiro[4.dec7-en-2-one

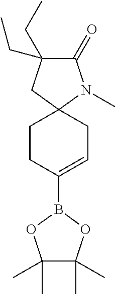 [SMILES: CCC1(CC)CC2(CC=C(B3OC(C)(C)C(C)(C)O3)CC2)N(C)C1=O]

To a three neck RBF charged with 3,3-diethyl-1-methyl-2-oxo-1-azaspiro[4.5]dec-7-en-8-yl trifluoromethanesulfonate (0.5 g, 1.3 mmol), bispinacolato diborane (0.34 g, 1.3 mmol) and potassium acetate (0.4 g, 4.07 mmol) in 1,4-dioxane (5 ml) and purged with argon gas for 30 min. PdCl₂(dppf) (0.99 g, 0.135 mmol) was added in reaction mixture and heated at 70° C. for 1 h. The reaction mixture was diluted with water and extracted with ethyl acetate (2×20 ml). The combined organic layers were dried over anhydrous sodium sulfate and concentrated under reduced pressure. The crude product was purified by column chromatography. The product was eluted in 50% ethyl acetate in n-hexane gives solid title compound (85.2%). ¹H-NMR (400 MHz, CDCl₃) δ: 6.49 (s, 1H), 2.75 (s, 3H), 2.53-2.48 (m, 2H), 2.10-1.66 (m, 6H), 1.60-1.52 (q, 4H), 1.29-1.26 (s, 12H), 0.9-0.8 (t, 6H).

### Step-11: Synthesis of 3-(3,3-diethyl-1-methyl-2-oxo-1-azaspiro[4.5]dec-7-en-8-yl)-1-(tetrahydro-2H-pyran-2yl)-1H-pyrazol-4-carbaldehyde

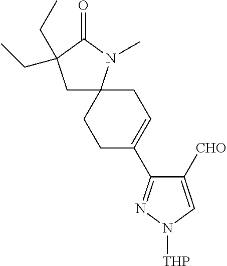

To a 3 ml sealed tube was charged 3,3-diethyl-methyl-8-(4,4,5,5-tetramethyl-1,3,2-dioxaborolan-2-yl)-1-azaspiro[4.dec7-en-2-one (0.4 g, 1.15 mmol) with 3-iodo-1-(tetrahydro-2H-pyran-2-yl)-1H-pyrazole-4-carbaldehyde (0.387 g, 1.26 mmol) in 1,4 dioxane:water mixture (9:1), and purged with argon gas for 30 min. Then was added with potassium carbonate (0.733, 3.46 mmol), PdCl₂(dppf) (0.084 g, 0.115 mmol). The reaction mixture was heated at 70° C. for 2 h. The reaction mixture was diluted with water and extracted with ethyl acetated (2×10 ml), combined organic layer was dried over anhydrous sodium sulfate and concentrate. The crude material was purified by column chromatography using mobile phase in 3% MeOH in DCM gives title compound (43.5%). ¹H-NMR (400 MHz, CDCl₃) δ: 9.92 (s, 1H), 8.18 (s, 1H), 6.41 (t, 1H), 5.38-5.32 (t, 1H), 4.17-4.11 (t, 2H), 2.84-2.82 (s, 3H), 2.79-1.53 (m, 14H), 1.30-1.25 (q, 4H), 0.93-0.84 (t, 6H).

### Step-12: Synthesis of 3-(3,3-diethyl-1-methyl-2-oxo-1-azaspiro[4.5]dec-7-en-8-yl)-1H-pyrazole-4)-carbaldehyde

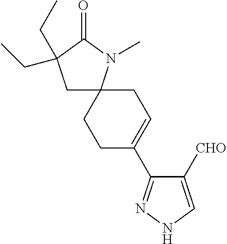 [SMILES: CCC1(CC)CC2(CC=C(c3n[nH]cc3C=O)CC2)N(C)C1=O]

To a stirred solution of 3-(3,3-diethyl-1-methyl-2-oxo-1-azaspiro[4.5]dec-7-en-8-yl)-1-(tetrahydro-2H-pyran-2yl)-1H-pyrazol-4-carbaldehyde (0.2 g, 0.501 mmol) in MeOH (2 ml) at rt was added HCl (conc. 4 ml), and the resulting mixture was stirred reaction 2 h at rt. The reaction mixture was diluted in water and extracted with DCM (3×20 ml), washed with sodium bicarbonate solution, and the combined organic layer was dried over Na₂SO₄. The solvent was removed under reduced pressure and the resulting crude was purified by column chromatography using mobile phase elute 3% methanol in dichloromethane to give title compound (63.7%) was: LCMS: m/z=316.27 [M+1].

### Step-13: Synthesis of tert-butyl(2((3-(3,3-diethyl-1-methyl-2-oxo-1-azaspiro[4.5]dec-7-en-8-yl)-1H-pyrazol-4-yl)(methyl)(methyl)amino)ethyl)(methyl)carbamate

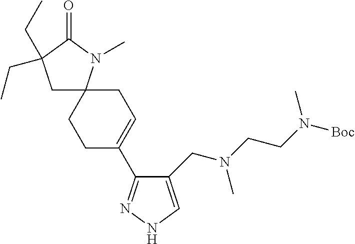 [SMILES: CCC1(CC)CC2(CC=C(c3n[nH]cc3CN(C)CCN(C)C(=O)OC(C)(C)C)CC2)N(C)C1=O]

To a stirred solution of 3-(3,3-diethyl-1-methyl-2-oxo-1-azaspiro[4.5]dec-7-en-8-yl)-1H-pyrazole4)carbaldehyde (0.1 g, 0.332 mmol) and tertbutyl(2methylamino)ethyl)carbamate (0.093 g, 0.497 mmol) in EDC (4 ml) at rt, were added sodium triacetoxy borohydride (0.210 g, 0.995 mmol) in portion wise, stirred reaction 1 h at 60° C. The reaction was diluted by water and extracted with DCM (2×15 ml), washed with sodium bicarbonate solution, the combined organic layers were dried over anhydrous sodium sulphate. The solvent was removed under reduced pressure and the resulting crude was purified by column chromatography using mobile phase 5% methanol in dichloromethane to give title compound (64.9%). LCMS: m/z=487.35 [M+1].

### Step-14: Synthesis of tert-butyl(2((3-(3,3-diethyl-1-methyl-2-oxo-1-azaspiro[4.5]decan-8-yl)-1H-pyrazol-4-yl)(methyl)(methyl)amino)ethyl)(methyl)carbamate

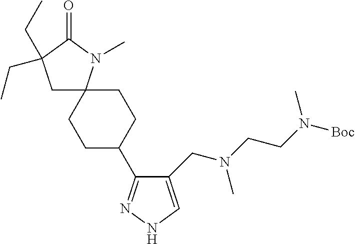 [SMILES: CCC1(CC)CC2(CCC(c3n[nH]cc3CN(C)CCN(C)C(=O)OC(C)(C)C)CC2)N(C)C1=O]

To a stirred solution of tert-butyl(2((3-(3,3-diethyl-1-methyl-2-oxo-1-azaspiro[4.5]dec-7-en-8-yl)-1H-pyrazol-4-yl)(methyl)(methyl)amino)ethyl)(methyl)carbamate (80 mg, 0.0016 mmol) in IPA in HCl (2 ml) at rt, was added 20% palladium hydroxide (0.1 g). After purging with H₂ for 1 h, the reaction mixture was passed through Celite and washed with MeOH, The solvent was removed under reduced pressure and the resulting crude was purified by Prep HPLC using mobile phase A: 0.1% TFA in water, B: 100% ACN (80%). LCMS: m/z=490.7 [M+1].

### Step-15: Synthesis of 3,3-diethyl-1-methyl-8-(4-((methyl(2-methylamino)ethyl)amino)methyl)-1H-pyrazol-3-yl)-1-azaspiro[4.5]decan-2-one

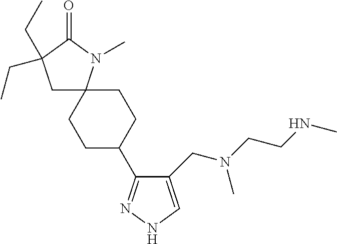 [SMILES: CCC1(CC)CC2(CCC(c3n[nH]cc3CN(C)CCNC)CC2)N(C)C1=O]

To a stirred solution tert-butyl(2((3-(3,3-diethyl-1-methyl-2-oxo-1-azaspiro[4.5]decan-8-yl)-1H-pyrazol-4-yl)(methyl)(methyl)amino)ethyl)(methyl)carbamate (0.080 g, 0.00016 mmol) in IPA-HCl (2 ml) at rt for 24 h, the reaction mixture was removed under reduced pressure. The residue obtained was triturated with pentane to give title compound as HCl salt (95.2%). ¹H-NMR (400 MHz, D₂O) δ: 7.79 (s, 1H), 4.31 (s, 2H), 3.78-3.44 (m, 4H), 2.75 (s, 3H), 2.69 (m, 7H), 1.89 (s, 2H), 1.85-1.79 (m, 4H), 1.70-1.67 (t, 2H), 1.51-1.48 (t, 2H) 1.43-1.38 (q, 4H), 0.71-0.68 (t, 6H).

### Example B-16. Synthesis of N1,N2-dimethyl-N1-((3-((1s,4s)-4-((2,2,2-trifluoroethoxy)methyl)cyclohexyl)-1H-pyrazol-4-yl)methyl)ethane-1,2-diamine and N1,N2-dimethyl-N1-((3-((1r,4r)-4-((2,2,2-trifluoroethoxy)methyl)cyclohexyl)-1H-pyrazol-4-yl)methyl)ethane-1,2-diamine and N1,N2-dimethyl-N1-((3-(4-((2,2,2-trifluoroethoxy)methyl)cyclohex-1-en-1-yl)-1H-pyrazol-4-yl)methyl)ethane-1,2-diamine (Compounds 23, 24, and 16)

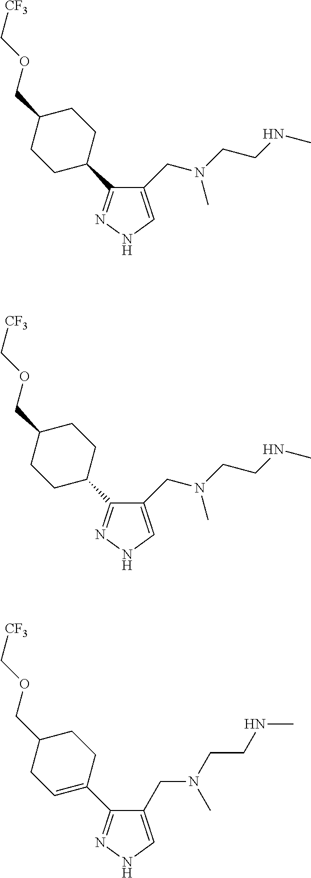 [SMILES: CNCCN(C)Cc1c[nH]nc1[C@H]1CC[C@@H](COCC(F)(F)F)CC1 ; CNCCN(C)Cc1c[nH]nc1[C@H]1CC[C@H](COCC(F)(F)F)CC1 ; CNCCN(C)Cc1c[nH]nc1C1=CCC(COCC(F)(F)F)CC1]

### Step-1: Synthesis of ethyl 1,4-dioxaspiro[4.5]decane-8-carboxylate

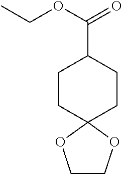

To a three neck RBF equipped with Dean-Stark apparatus, ethylene glycol (5.55 g, 89.6 mmol) and p-toluene sulphonic acid (0.15 g, 6 mmol) were added to a solution of ethyl 4-oxocyclohexane-1-carboxylate (10.0 g, 58.8 mmol) in toluene (30 ml) and the mixture was stirred and refluxed at 140° C. for 2 h. Water (80 ml) was added to the reaction mixture, stirred for 2-5 min. and then the aqueous layer was extracted with diethyl ether (3×50 ml). The combined organic extracts were washed with NaHCO₃ solution (sat.) and brine, dried over Na₂SO₄ filtered and evaporated under reduced pressure to get the title compound as a faint yellow viscous liquid. (88%). ¹H NMR (400 MHz, CDCl₃) δ: 4.15-4.10 (q, 2H), 3.95 (s, 4H), 2.38-2.30 (m, 1H), 1.97-1.92 (m, 2H), 1.85-1.76 (m, 4H), 1.60-1.52 (m, 2H), 1.27-1.23 (t, 3H).

### Step-2: Synthesis of (1,4-dioxaspiro[4.5]decan-8-yl)methanol

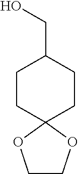 [SMILES: OCC1CCC2(CC1)OCCO2]

A solution of ethyl 1,4-dioxaspiro[4.5]decane-8-carboxylate in tetrahydrofuran at 0° C. was treated dropwise with LAH (1.0M solution in tetrahydrofuran). The reaction mixture was stirred at 0° C. for 30 min and quenched by dropwise addition of ethyl acetate. The combined organic extracts were dried over sodium sulphate, filtered and then concentrated in vacuum to afford pure (1, 4-dioxaspiro [4.5]decan-8-yl)methanol as a pale yellow oil. ¹H NMR (400 MHz, CDCl₃) δ: 3.99-3.93 (m, 4H), 3.85-3.78 (q, 2H), 3.47-3.45 (d, J=4.8 Hz, 2H), 1.83-1.76 (m, 2H), 1.73-1.52 (m, 3H), 1.39-1.35 (m, 2H).

### Step-3: Synthesis of (1,4-dioxaspiro[4.5]decan-8-yl)methyl methanesulfonate

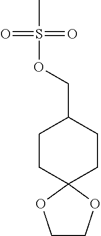 [SMILES: CS(=O)(=O)OCC1CCC2(CC1)OCCO2]

To a solution of (1,4-dioxaspiro[4.5]decan-8-yl)methanol (0.3 g, 0.0017 mol) in methylene chloride (3 ml) at 0-5° C., triethylamine (0.53 g, 0.73 ml, 0.0052 mol) was added and stirred for 15 min. Finally methanesulfonyl chloride (0.3 g, 0.2 ml, 0.0026 mol) was added dropwise to the reaction mixture and allowed to stir at rt for 3 h. Water was added and extracted with ethyl acetate. The organic layer was evaporated and crude material was purified by column chromatography using mobile phase 0-3% ethyl acetate in hexane gives title compound as pale yellow viscous oil (91.7%). ¹H NMR (400 MHz, CDCl₃) δ: 4.07-4.06 (d, J=7.6 Hz, 2H), 3.98-3.91 (m, 4H), 3.01 (s, 3H), 1.83-1.77 (m, 5H), 1.60-1.52 (m, 2H), 1.40-1.30 (m, 2H).

### Step-4: Synthesis of 8-((2,2,2-trifluoroethoxy)methyl)-1,4-dioxaspiro[4.5]decane

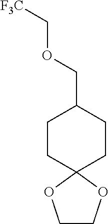 [SMILES: FC(F)(F)COCC1CCC2(CC1)OCCO2]

Sodium hydride (60% in mineral oil, 275 mg, 6.88 mmol) was washed with pentane and then suspended in dry THF (2 ml) under nitrogen atmosphere. A solution of 2,2,2-trifluoroethanol (0.110 g, 6.88 mmol) in dry THF (1 ml) was added, and the resulting mixture was stirred for 30 min at rt. A solution of (1,4-dioxaspiro[4.5]decan-8-yl)methyl methanesulfonate (250 mg, 1.05 mmol) in dry THF (1 ml) was added and the reaction was heated at reflux for 50 h. The reaction was cooled to room temperature, and a saturated solution of NH₄Cl (10 ml) was added slowly. The mixture was concentrated to remove THF. Residue obtained was diluted with water and extracted with ethyl acetate and the solvent was concentrated under reduced pressure to give title compound (35.4%) ¹H NMR (400 MHz, CDCl₃) δ: 3.99-3.93 (m, 4H), 3.85-3.78 (q, 2H), 3.47-3.45 (d, J=6.4 Hz, 2H), 1.83-1.77 (m, 4H), 1.60-1.52 (m, 3H), 1.39-1.35 (m, 2H).

### Step-5: Synthesis of 8-4-((2,2,2-trifluoroethoxy)methyl)cyclohexan-1-one

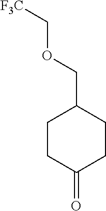 [SMILES: O=C1CCC(COCC(F)(F)F)CC1]

In to a 10 ml round bottom flask, was placed 8-((2,2,2-trifluoroethoxy)methyl)-1,4-dioxaspiro[4.5]decane (0.1 g, 0.00039 mol), tetrahydrofuran (2 ml), hydrochloric solution (3M, 2 ml). The resulting solution was stirred for 4 h at rt. THF was removed under vacuum. The resulting solution was extracted with ethyl acetate. The combined organic layers was washed with saturated solution of Na₂CO₃ and then concentrated under vacuum to obtain (96.8%) ¹H NMR (400 MHz, CDCl₃) δ: 3.88-3.82 (q, 2H), 3.56-3.54 (d, J=6.4 Hz, 2H), 2.47-2.18 (m, 4H), 2.17-2.06 (m, 3H), 1.54-1.41 (m, 2H).

### Step-6: Synthesis of 4-((2,2,2-trifluoroethoxy)methyl)cyclohex-1-en-1-yl trifluoromethanesulfonate

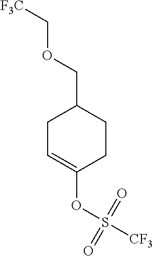 [SMILES: O=S(=O)(OC1=CCC(COCC(F)(F)F)CC1)C(F)(F)F]

In a 10 ml round bottom flask, 8-4-((2,2,2-trifluoroethoxy)methyl)cyclohexan-1-one (0.1 g, 0.00048 mol), dichloromethane (4 ml) and 2,6-di-tert-butyl-4-methylpyridine (0.110 g, 0.00054 mol). The resulting solution was stirred under a static argon atmosphere and cooled to 0° C., at which time the dropwise addition of (0.15 g, 0.00053 mol) of trifluoromethanesulfonic anhydride was started. After complete addition, the brown mixture is allowed to warm slowly to room temperature and was stirred at that temperature for 2 h. The solvent was removed by distillation and the resulting light tan material was treated with 25 ml pentane and heated to reflux for 10 min. The tan salt thus obtained were removed by filtration and washed with 25 ml portion of pentane. The combined pentane solution was distilled and passed through silica to obtained the title compound as a yellow oil product (79.8%) ¹H NMR (400 MHz, CDCl₃) δ: 5.78-5.77 (t, J=4.4 Hz, 1H), 3.87-3.81 (q, J=Hz, 2H), 3.55-3.54 (d, J=5.6 Hz, 2H), 2.48-2.32 (m, 3H), 2.04-1.96 (m, 3H), 1.60-1.57 (m, 1H).

### Step-7: Synthesis of 4,4,5,5-tetramethyl-2-(4-((2,2,2-trifluoroethoxy)methyl)cyclohex-1-en-1-yl)-1,3,2-dioxaborolane

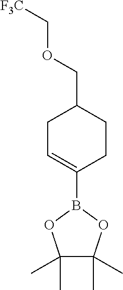 [SMILES: CC1(C)OB(C2=CCC(COCC(F)(F)F)CC2)OC1(C)C]

In a 10 ml round bottom flask charged 4-((2,2,2-trifluoroethoxy)methyl)cyclohex-1-en-1-yl trifluoromethanesulfonate (0.13 g, 0.00038 mol), potassium acetate (0.112 g, 0.00114 mol), 4,4,4′,4′,5,5,5′,5′-octamethyl-2,2′-bi(1,3,2-dioxaborolane) (0.096 g, 0.00038 mol) and 1,1′-bis(diphenylphosphino)ferrocene]dichloropalladium(II) (0.028 g, 0.000038 mol) and 1,4-dioxane (3 ml). The resulting solution was stirred under argon degassing for 20 min. resulting mixture was heated at 80° C. for 2 h. After heating, resulting brown mass was concentrated and placed on silica gel bed for column chromatography. Pure pale yellow oil compound was eluted at 0-1.2% ethyl acetate in hexane (32.9%). ¹H NMR (400 MHz, CDCl₃) δ: 6.56-6.56 (d, J=2.0 Hz, 1H), 3.86-3.79 (q, J=8.8 Hz, 2H), 3.51-3.49 (d, J=6.4 Hz, 2H), 2.27-2.22 (d, J=16.8 Hz, 2H), 2.15-2.11 (m, 1H), 1.96-1.80 (m, 3H), 1.28 (s, 12H).

### Step-8: Synthesis of 1-(tetrahydro-2H-pyran-2-yl)-3-(4-((2,2,2-trifluoroethoxy)methyl)cyclohex-1-en-1-yl)-1H-pyrazole-4-carbaldehyde

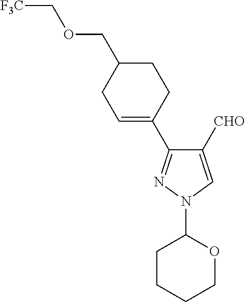 [SMILES: O=Cc1cn(C2CCCCO2)nc1C1=CCC(COCC(F)(F)F)CC1]

In a 10 ml 3-necked RBF, 4,4,5,5-tetramethyl-2-(4-((2,2,2-trifluoroethoxy)methyl)cyclohex-1-en-1-yl)-1,3,2-dioxaborolane (0.1 g, 0.3123 mmol), 1,4-dioxane (2 ml) and water (0.5 ml). Resulting solution was stirred under static argon for 30 min at which time potassium phosphate (0.198 g, 0.937 mmol), 3-iodo-1-(tetrahydro-2H-pyran-2-yl)-1H-pyrazole-4-carbaldehyde (0.124 g, 0.4059 mmol) was added in to the resulting solution. Finally, 1,1′-Bis(diphenylphosphino)ferrocene]dichloropalladium(II) (0.023 g, 0.0312 mmol) was placed in to the solution. Again argon degassing was kept for 10 min and finally resulting mixture was heated at 70-75° C. for 2 h. After heating, the resulting brown mass was concentrated. To the resultant mass, water (10 ml) was added and then ethyl acetate (2×15 ml) was added for extraction. The organic layers were concentrated and placed over silica gel to purify. Pure product was eluted at 9% ethyl acetate in hexane. (59.9). ¹H NMR (400 MHz, CDCl₃) δ: 9.92 (s, 1H), 8.15 (s, 1H), 6.30 (s, 1H), 5.40-5.37 (m, 1H), 4.13-4.10 (d, 1H), 3.90-3.83 (q, J=8.8 Hz, 2H), 3.76-3.71 (m, 1H), 2.69-2.64 (d, 1H), 2.42-2.38 (d, 1H), 2.11-2.07 (d, 1H), 2.03-1.98 (m, 5H), 1.75-1.65 (m, 4H), 1.52-1.42 (m, 2H).

### Step-9: Synthesis of 3-(4-((2,2,2-trifluoroethoxy)methyl)cyclohex-1-en-1-yl)-1H-pyrazole-4-carbaldehyde

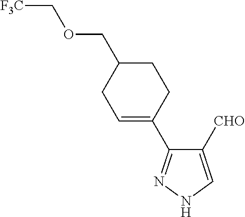 [SMILES: O=Cc1c[nH]nc1C1=CCC(COCC(F)(F)F)CC1]

1-(tetrahydro-2H-pyran-2-yl)-3-(4-((2,2,2-trifluoroethoxy)methyl)cyclohex-1-en-1-yl)-1H-pyrazole-4-carbaldehyde (0.490 g, 1.31 mmol) dissolved in to methanol (4 ml) at 0° C. and Concentrated HCl (4 ml) was slowly added. The reaction mixture was stirred for 16 h. Solvent was evaporated to obtain crude residue of title compound (42.2%). LCMS: m/z=389.29 [M+1].

### Step-10: Synthesis of tert-butyl methyl(2-(methyl((1-(tetrahydro-2H-pyran-2-yl)-3-(4-((2,2,2-trifluoroethoxy)methyl)cyclohex-1-en-1-yl)-1H-pyrazol-4-yl)methyl)amino)ethyl)carbamate

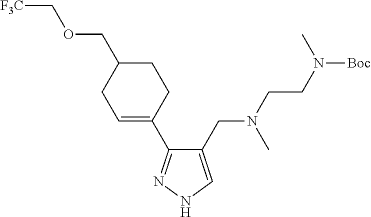 [SMILES: CN(CCN(C)C(=O)OC(C)(C)C)Cc1c[nH]nc1C1=CCC(COCC(F)(F)F)CC1]

To a 3-neck 10 ml RBF, 3-(4-((2,2,2-trifluoroethoxy)methyl)cyclohex-1-en-1-yl)-1H-pyrazole-4-carbaldehyde (0.160 g, 0.55 mmol) and tert-butyl methyl(2-(methylamino)ethyl)carbamate (0.157 g, 0.83 mmol) was dissolved in ethylene dichloride (4 ml) at 5° C. temperature. Sodium triacetoxy borohydride (0.353 g, 1.66 mmol) was added portionwise. After addition, the ice bath was removed and reaction mixture was stirred at 50-55° C. for 2 h. Sodium bicarbonate (sat. aq.) was added to reaction mixture and extracted by DCM (3×20 ml), dried over Na₂SO₄. The organic layers were concentrated and the crude product was purified by silica gel column chromatography using mobile phase 0-16% ethyl acetate in hexane to get the title compound as a pale yellow liquid. (85.46%). LCMS: m/z=461.66 [M+1].

### Step-11: Synthesis of tert-butylmethyl(2-(methyl((3-((1r,4r)-4-((2,2,2-trifluoroethoxy)methyl)cyclohexyl)-1H-pyrazol-4-yl)methyl)amino)ethyl)carbamate and tert-butyl methyl(2-(methyl((3-((1s,4s)-4-((2,2,2-trifluoroethoxy)methyl)cyclohexyl)-1H-pyrazol-4-yl)methyl)amino)ethyl)carbamate

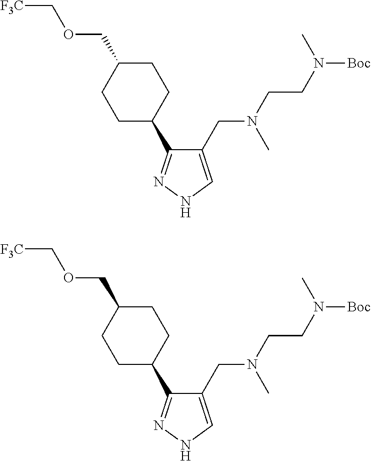 [SMILES: CN(CCN(C)C(=O)OC(C)(C)C)Cc1c[nH]nc1[C@H]1CC[C@H](COCC(F)(F)F)CC1 ; CN(CCN(C)C(=O)OC(C)(C)C)Cc1c[nH]nc1[C@H]1CC[C@@H](COCC(F)(F)F)CC1]

To a stirred solution of tert-butyl methyl(2-(methyl((1-(tetrahydro-2H-pyran-2-yl)-3-(4-((2,2,2-trifluoroethoxy)methyl)cyclohex-1-en-1-yl)-1H-pyrazol-4-yl)methyl)amino)ethyl)carbamate (0.2 g, 4.3 mmol) in methanol (4 ml), palladium hydroxide (0.05 g) was added and the reaction flask was purged with H₂ for 45 min. The reaction mixture was then diluted with 20% EtOAc in methanol (20 ml) and filtered through Celite. The Celite was washed with 20% EtOAc in methanol (2×10 ml). The solvent was concentrated to get product crude compound containing two regioisomers (59.7%). LCMS: m/z=463.6 [M+1]. The isomers were separated by prep-HPLC using chiralcel 02H (250×4.6) mm, 5 column and 0.1% TFA in n-hexane and 0.1% TFA in ethanol mobile phase to afford two fractions. Fraction 1 (yield: 49 mg), Fraction 2 (yield: 56 mg).

### Step-12: Synthesis of N1,N2-dimethyl-N1-((3-((1s,4s)-4-((2,2,2-trifluoroethoxy)methyl)cyclohexyl)-1H-pyrazol-4-yl)methyl)ethane-1,2-diamine

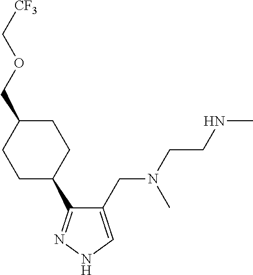 [SMILES: CNCCN(C)Cc1c[nH]nc1[C@H]1CC[C@@H](COCC(F)(F)F)CC1]

tert-butyl methyl(2-(methyl((3-((1s,4s)-4-((2,2,2-trifluoroethoxy)methyl)cyclohexyl)-1H-pyrazol-4-yl)methyl)amino)ethyl)carbamate (49 mg, 0.011 mmol) in 4M HCl in isopropanol (4 ml) was stirred for 48 h at rt. The reaction mixture was concentrated under vacuum to obtain a pale yellow solid residue which was triturated with n-pentane and diethyl ether to afford the dihydrochloride salt of the title compound (65.1%) as off-white solid. LCMS: m/z=363.23 [M+1]. ¹H NMR (400 MHz, D₂O) δ: 7.58 (s, 1H), 4.21 (s, 2H), 3.84-3.79 (q, J=9.2 Hz, 2H), 3.38-3.33 (m, 6H), 2.64 (s, 3H), 2.58 (m, 4H), 1.70-1.67 (m, 4H), 1.54 (m, 3H), 1.40-1.30 (m, 2H).

### Step-13: Synthesis of N1,N2-dimethyl-N1-((3-((1r,4r)-4-((2,2,2-trifluoroethoxy)methyl)cyclohexyl)-1H-pyrazol-4-yl)methyl)ethane-1,2-diamine

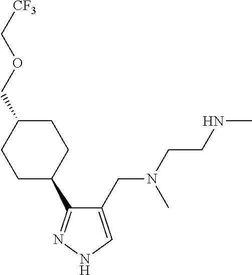 [SMILES: CNCCN(C)Cc1c[nH]nc1[C@H]1CC[C@H](COCC(F)(F)F)CC1]

tert-butylmethyl(2-(methyl((3-((1 r,4r)-4-((2,2,2-trifluoroethoxy)methyl)cyclohexyl)-1H-pyrazol-4-yl)methyl)amino)ethyl)carbamate (56 mg, 0.0121 mmol) in 4M HCl in isopropanol (4 ml) was stirred for 48 h at room temperature. The reaction mixture was concentrated under vacuum to obtain a pale yellow solid residue which was triturated with n-pentane and diethyl ether to afford the dihydrochloride salt of the title compound (72.9%) as an off-white solid. LCMS: m/z=363.23 [M+1]. ¹H NMR (400 MHz, D₂O) δ: 7.69 (s, 1H), 4.27 (s, 2H), 3.96-3.89 (m, 2H), 3.71-3.69 (d, J=7.6 Hz, 2H), 3.42 (s, 4H), 2.73 (s, 4H), 2.67 (m, 3H), 1.96 (bs, 1H), 1.63-1.55 (m, 8H).

### Step-14: Synthesis of N1,N2-dimethyl-N1-((3-(4-((2,2,2-trifluoroethoxy)methyl)cyclohex-1-en-1-yl)-1H-pyrazol-4-yl)methyl)ethane-1,2-diamine

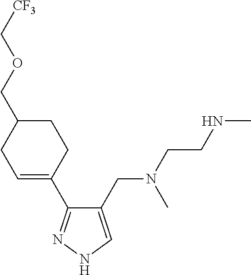 [SMILES: CNCCN(C)Cc1c[nH]nc1C1=CCC(COCC(F)(F)F)CC1]

To a solution of tert-butyl methyl(2-(methyl((1-(tetrahydro-2H-pyran-2-yl)-3-(4-((2,2,2-trifluoroethoxy)methyl)cyclohex-1-en-1-yl)-1H-pyrazol-4-yl)methyl)amino)ethyl)carbamate (0.050 g, 0.092 mmol) in MeOH (1 ml) HCl in IPA (1.5 ml) was added. The resultant solution was stirred for 5 h at rt. The reaction solvent was concentrated under reduced pressure which was subjected to Prep HPLC using column Phenomenex Gemini (150 mm×20 mm×5 μm) in 0.1% TFA in water/ACN mobile phase to afford an off-white solid (96.7%). LCMS: m/z=361.44 [M+1]. ¹H NMR (400 MHz, MeOH) δ: 7.82 (s, 1H), 6.05 (s, 1H), 4.43 (s, 2H), 3.98-3.92 (q, J=9.2 Hz, 2H), 3.61-3.59 (d, J=5.2 Hz, 2H), 3.51 (s, 4H), 2.84 (s, 3H), 2.79 (s, 3H), 2.48-2.39 (m, 3H), 2.03-2.01 (m, 3H), 1.56-1.46 (m, 1H).

### Example B-17. Synthesis of N1-((3-((5s,8s)-3,3-dimethyl-1-oxaspiro[4.5]decan-8-yl)-1H-pyrazol-4-yl)methyl)-N2-methylethane-1,2-diamine and N1-((3-((5r,8r)-3,3-dimethyl-1-oxaspiro[4.5]decan-8-yl)-1H-pyrazol-4-yl)methyl)-N2-methylethane-1,2-diamine (Compounds 28 and 29)

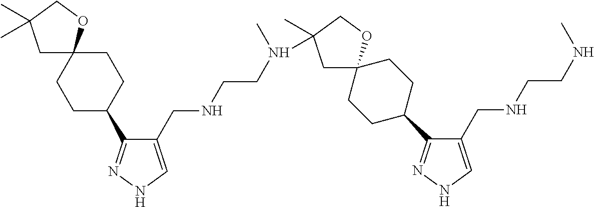

### Step-1: Synthesis of 2-(3,3-dimethyl-1-oxaspiro[4.5]dec-7-en-8-yl)-4,4,5,5-tetramethyl-1,3,2-dioxaborolane

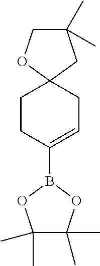

In a 50 ml 3-necked round bottom flask charged with 3,3-dimethyl-1-oxaspiro[4.5]dec-7-en-8-yl trifluoromethanesulfonate (3.0 g, 9.54 mmol) potassium acetate (2.81 g, 428.6 mmol), 4,4,4′,4′5,5,5′,5′-octamethyl-2,2′-bi(1,3,2-dioxaborolane) (2.43 g, 9.54 mmol) Pd(dppf)Cl₂ (0.698 g, 0.95 mmol) and 1,4-dioxane (25 ml). The resulting solution was stirred under argon degassing for 20 min. The reaction was heated at 80° C. for 2 h. After heating, the resulting brown mass was concentrated and the residue obtained was placed on silica gel bed for column chromatography using mobile phase 0-2% ethyl acetate in hexane as pale yellow oil (43.4%).

### Step-2: Synthesis of 3-(3,3-dimethyl-1-oxaspiro[4.5]dec-7-en-8-yl)-1-(tetrahydro-2H-pyran-2-yl)-1H-pyrazole-4-carbaldehyde

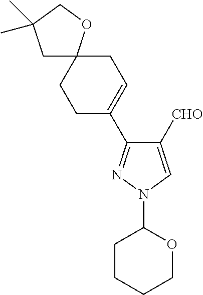 [SMILES: CC1(C)COC2(CC=C(c3nn(C4CCCCO4)cc3C=O)CC2)C1]

In a 10 ml three neck RBF, charged 2-(3,3-dimethyl-1-oxaspiro[4.5]dec-7-en-8-yl)-4,4,5,5-tetramethyl-1,3,2-dioxaborolane (1.21 g, 4.14 mmol), cesium carbonate (4.03 g, 12.42 mmol), 3-iodo-1-(tetrahydro-2H-pyran-2-yl)-1H-pyrazole-4-carbaldehyde (1.65 g, 5.38 mmol) and 1,4-dioxane (15 ml) and water (1.5 ml). The resulting solution was stirred under argon for 30 min and added Pd(dppf)Cl₂ (0.302 g, 0.414 mmol). Again argon degassing was kept for 10 min and finally the resulting mixture was heated at 75° C. for 2 h. The resulting brown mass was concentrated, water (30 ml) was added and the resulting mixture was extracted by ethyl acetate (3×25 ml). The organic layers were dried over sodium sulphate and concentrated under reduced pressure. The crude material was purified by column chromatography using mobile phase 0-18% ethyl acetate in hexane to give title compound (38.6%). LCMS: m/z=345.4 [M+1].

### Step-3: Synthesis of 3-(3,3-dimethyl-1-oxaspiro[4.5]dec-7-en-8-yl)-1H-pyrazole-4-carbaldehyde

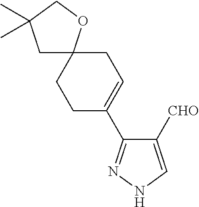 [SMILES: CC1(C)COC2(CC=C(c3n[nH]cc3C=O)CC2)C1]

To a 25 ml flask, 3-(3,3-dimethyl-1-oxaspiro[4.5]dec-7-en-8-yl)-1-(tetrahydro-2H-pyran-2-yl)-1H-pyrazole-4-carbaldehyde (1.2 g, 3.48 mmol) was dissolved in to methanol (10 ml) at 0-5° C. HCl (conc. 8 ml) was slowly added. The reaction mixture was stirred at rt for 16 h. The reaction mixture was concentrated the under reduced pressure to afford crude product which was purified by 0-25% ethyl acetate in hexane to give the title compound as a pale yellow oil (49.62%). LCMS: m/z=261.3 [M+1].

### Step-4: Synthesis of tert-butyl (2-(((3-(3, 3-dimethyl-1-oxaspiro [4.5]dec-7-en-8-yl)-1H-pyrazol-4-yl)methyl)amino)ethyl)(methyl)carbamate

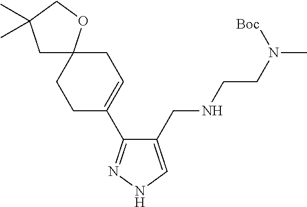

To a 3-necked 25 ml RBF, 3-(3,3-dimethyl-1-oxaspiro[4.5]dec-7-en-8-yl)-1H-pyrazole-4-carbaldehyde (0.240 g, 0.92 mmol) and tert-butyl (2-aminoethyl)(methyl)carbamate (0.241 g, 1.38 mmol) was dissolved in EDC (6 ml) at 0° C. Sodium triacetoxy borohydride (0.586 g, 2.76 mmol) was added portionwise. After addition, the ice bath was removed and the reaction mixture was stirred at 55° C. for 2 h. The reaction mixture was neutralized by sodium bicarbonate (sat. 20 ml) and extracted by DCM (3×25 ml), dried over Na₂SO₄. The solvent was evaporated to obtain crude product which was purified using silica column chromatography using mobile phase 0-2% MeOH in DCM to afford the title compound as a thick pale yellow oil (29.9%). LCMS: m/z=419.5 [M+1].

### Step-5: Synthesis of tert-butyl (2-(((3-((5r,8r)-3,3-dimethyl-1-oxaspiro[4.5]decan-8-yl)-1H-pyrazol-4-yl)methyl)amino)ethyl)(methyl)carbamate and tert-butyl (2-(((3-(3,3-dimethyl-1-oxaspiro[4.5]decan-8-yl)-1H-pyrazol-4-yl)methyl)amino)ethyl)(methyl)carbamate

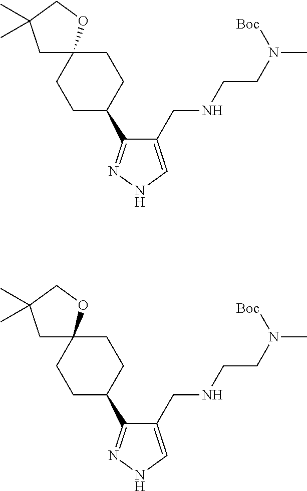

To a stirred solution of tert-butyl (2-(((3-(3,3-dimethyl-1-oxaspiro[4.5]dec-7-en-8-yl)-1H-pyrazol-4-yl)methyl)amino)ethyl)(methyl)carbamate (0.25 g, 0.598 mmol) in THF (5 ml), 20% Palladium hydroxide (0.08 g) was added and the reaction flask was purged with H₂ for 2 h. The reaction mixture was diluted with 20% EtOAc in methanol (10 ml) and filtered through celite bed and washed with 20% EtOAc in methanol (2×10 ml), the filtrate was concentrated under reduced pressure to afford title compound. (79.9%). The isomers were separated by prep-HPLC using Sunfire C18 (250×19 mm×5 μm), column and 0.1% TFA in water and 100% acetonitrile mobile phase to afford two fractions: Fraction 1 (80 mg), Fraction 2 (110 mg). LCMS: m/z=421.70 [M+1].

### Step-6: Synthesis of N1-((3-((5s,8s)-3,3-dimethyl-1-oxaspiro[4.5]decan-8-yl)-1H-pyrazol-4-yl)methyl)-N2-methylethane-1,2-diamine

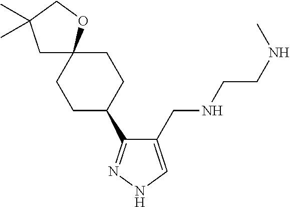 [SMILES: CNCCNCc1c[nH]nc1[C@H]1CC[C@@]2(CC1)CC(C)(C)CO2]

To a solution of tert-butyl (2-(((3-((5s,8s)-3,3-dimethyl-1-oxaspiro[4.5]decan-8-yl)-1H-pyrazol-4-yl)methyl)amino)ethyl)(methyl)carbamate (0.08 g, 0.19 mmol) in MeOH (2 ml) added HCl in IPA (2 ml). Evaporated solvents under reduced pressure to obtained colorless oil which was triturated using n-Pentane and diethyl ether to afford off-white solid (73.1%). LCMS: m/z=321.5 [M+1]. ¹H NMR (400 MHz, D₂O) δ: 7.69 (s, 1H), 4.05 (s, 2H), 3.30 (s, 2H), 3.23 (bs, 4H), 2.69 (s, 1H), 2.55 (s, 3H), 1.76-1.73 (d, 2H), 1.53-1.37 (m, 8H), 0.84 (s, 6H).

### Step-7: Synthesis of N1-((3-((5r,8r)-3,3-dimethyl-1-oxaspiro[4.5]decan-8-yl)-1H-pyrazol-4-yl)methyl)-N2-methylethane-1,2-diamine

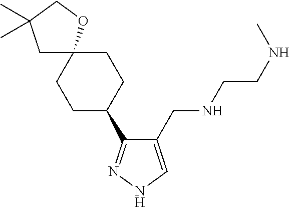 [SMILES: CNCCNCc1c[nH]nc1[C@H]1CC[C@]2(CC1)CC(C)(C)CO2]

To a solution of tert-butyl (2-(((3-((5r,8r)-3,3-dimethyl-1-oxaspiro[4.5]decan-8-yl)-1H-pyrazol-4-yl)methyl)amino)ethyl)(methyl)carbamate (0.11 g, 0.26 mmol) in MeOH (3 ml) added HCl in IPA (3 ml). Solvent was evaporated under reduced pressure to obtained off-white residue which was triturated using n-pentane and diethyl ether to afford off-white solid. (73.1%). LCMS: m/z=321.48 [M+1]. ¹H NMR (400 MHz, D₂O) δ: 7.54 (s, 1H), 3.93 (s, 2H), 3.22 (s, 4H), 3.12 (s, 2H), 2.55 (s, 1H), 2.44 (s, 3H), 1.58 (m, 4H), 1.45 (s, 2H), 1.23 (m, 4H), 0.76 (s, 6H).

### Example B-18. Synthesis of N1-((3-((1R,3r,5S)-1,5-diethyl-8-oxabicyclo[3.2.1]octan-3-yl)-1H-pyrazol-4-yl)methyl)-N1,N2-dimethylethane-1,2-diamine and N1-((3-((1R,3s,5S)-1,5-diethyl-8-oxabicyclo[3.2.1]octan-3-yl)-1H-pyrazol-4-yl)methyl)-N1,N2-dimethylethane-1,2-diamine (Compound 37 and 38)

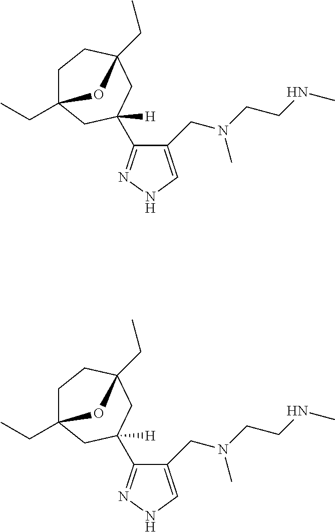

### Step-1: Synthesis of 2,5-diethylfuran

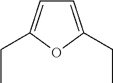

To a 3 L 3-neck round bottom flask under nitrogen atmosphere was added furan (15 g, 680 mmol) and TMEDA (56.1 g, 484 mmol) was added dropwise under cooling and stirring at 0° C. This mixture was added dropwise to n-BuLi (1.6M solution in hexane) at 0 to −10° C. After addition was complete, the reaction mixture was refluxed at 70° C. for 1 h. The reaction was then again cooled at 0° C. and treated with ethyl iodide (dissolved in 150 ml THF). The reaction mixture was then stirred at rt for 12 h. The reaction mixture was treated with a saturated solution of NH₄Cl and extracted with diethyl ether (3×200 ml). The separated organic layers were dried over anhydrous sodium sulphate and concentrated under reduced pressure to obtain yellow liquid. This crude material was purified by column chromatography in 60-120 mesh size silica. The expected compound was eluted in 100% DCM, concentrated to obtain yellow liquid (48.6%). ¹H NMR (400 MHz, CDCl₃) δ: 5.88 (s, 2H), 2.66-2.60 (q, J=8 Hz, 4H), 1.24-1.18 (t, 6H).

### Step-2: Synthesis of 2,2,4,4-tetrachloro-1,5-diethyl-8-oxabicyclo[3.2.1]oct-6-en-3-one

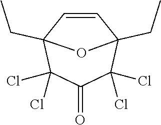 [SMILES: CCC12C=CC(CC)(O1)C(Cl)(Cl)C(=O)C2(Cl)Cl]

To another 100 ml round bottom flask was added trifluroethanol (90 ml) and treated with freshly prepared sodium metal (3.6 g) portionwise under nitrogen atmosphere at rt. This triflurosodiumethoxide solution was then added dropwise to 2,5-diethyl furan (13 g, 104.6 mmol) and pentachloroacetone (26.48 g, 115.15 mmol) in a 250 ml round bottom flask kept under nitrogen atmosphere at 0° C. Once addition was complete the ice bath was removed and stirring was continued for 3 h at rt. The reaction mixture was quenched with water (50 ml) and extracted with ethyl acetate (3×250 ml). The organic layer was dried over anhydrous sodium sulphate and concentrated to obtain crude compound. The crude material was purified by column chromatography. The desired product was eluted in 100% hexane to obtain colorless liquid (49.3%). ¹H NMR (400 MHz, CDCl₃) δ: 6.30 (s, 2H), 2.40-2.31 (m, 2H), 2.26-2.17 (m, 2H), 1.03-0.95 (q, 6H).

### Step-3: Synthesis of 1,5-diethyl-8-oxabicyclo[3.2.1]oct-6-en-3-one

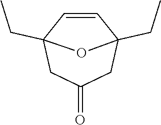

To a solution of 2,2,4,4-tetrachloro-1,5-diethyl-8-oxabicyclo[3.2.1]oct-6-en-3-one (16.5 g, 51.88 mmol) in methanol (180 ml) was saturated with ammonium chloride. To this was added with Zn—Cu couple (54.24 g, 83.01 mmol) portionwise and heated at 70° C. for 7 h. The reaction mixture was cooled to rt and stirred for another 15 h. The resulting suspension was filtered and concentrated. This residue was passed through silica bed so that salts were retained on silica to get the title product (98.9%). ¹H NMR (400 MHz, CDCl₃) δ: 5.97 (s, 2H), 2.48-2.44 (d, J=16.4 Hz, 2H), 2.36-2.32 (dd, J=16 Hz, 2H), 1.83-1.76 (m, 4H), 1.01-0.97 (t, 6H).

### Step-4: Synthesis of 1,5-diethyl-8-oxabicyclo[3.2.1]octan-3-one

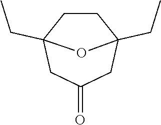

To a stirred solution of 2,2,4,4-tetrachloro-1,5-diethyl-8-oxabicyclo[3.2.1]oct-6-en-3-one (6 g, 33.2 mmol) in methanol (80 ml) was added with 10% Palladium on charcoal portion wise under nitrogen atmosphere and stirred it under hydrogen atmosphere for 2 h. The reaction mixture was filtered through Celite. The filtrate was concentrated to obtain viscous colorless (volatile) liquid. (71.1%) ¹H NMR (400 MHz, CDCl₃) δ: 2.42-2.31 (q, 4H), 1.77-1.68 (m, 10H), 0.97-0.93 (t, 3H).

### Step-5: Synthesis of 1, 5-diethyl-8-oxabicyclo[3.2.1]oct-2-en-3-yl trifluoromethanesulfonate

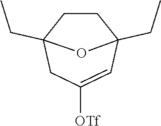 [SMILES: CCC12C=C(OS(=O)(=O)C(F)(F)F)CC(CC)(CC1)O2]

To a 250 ml round bottom flask fitted with nitrogen bubbler was charged with 1,5-diethyl-8-oxabicyclo[3.2.1]octan-3-one (4.3 g, 23.6 mmol) in THF (42 ml) and cooled at −78° C. This was treated with LiHMDS (35.4 ml, 35.4 mmol) dropwise and then stirred for 15 min followed by treatment with N-phenyl-O-((trifluoromethyl)sulfonyl)-N-(trifluoromethyl)sulfonyl)oxy)hydroxylamine (8.42 g, 23.6 mmol) dissolved in 15 ml THF. This was allowed to stirred for 30 min at −78° C. and then at rt for 2 h. The reaction mixture was quenched with saturated solution of NH₄Cl and separated with ethyl acetate (3×250 ml), and the organic layer was dried over anhydrous sodium sulphate and concentrated. The crude was purified by column chromatography. The product was eluted in 2% ethyl acetate in n-hexane (75%) ¹H NMR (400 MHz, CDCl₃) δ: 5.91 (s, 1H), 3.49 (d, 1H), 2.60-2.64 (d, J=16.4 Hz, 2H), 2.20-2.16 (d, J=16.4 Hz, 2H), 2.08-2.03 (m, 1H), 2.04-1.85 (m, 1H), 1.84-1.71 (m, 5H), 1.01-0.97 (m, 6H).

### Step-6: Synthesis of 2-(1,5-diethyl-8-oxabicyclo[3.2.1]oct-2-en-3-yl)-4,4,5,5-tetramethyl-1,3,2-dioxaborolane

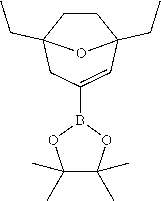 [SMILES: CCC12C=C(B3OC(C)(C)C(C)(C)O3)CC(CC)(CC1)O2]

To a 250 ml round bottom flask charged with 1,5-diethyl-8-oxabicyclo[3.2.1]oct-2-en-3-yl trifluoromethanesulfonate (5.5 g, 17.4 mmol), was added bispinacolatodiborane (4.41 g, 17.4 mmol), potassium acetate (5.15 g, 52.4 mmol) in 1,4 dioxane (80 ml) and purged with argon gas for 30 min. This was treated with Pd(dppf)Cl₂ (1.27 g, 1.75 mmol) and the reaction mixture was heated at 80° C. for 4 h. The reaction mixture was diluted with ethyl acetate and filtered over Celite, washed with water (100 ml). The combined organic layers were dried over anhydrous sodium sulfate and concentrated to obtain brown liquid. The crude compound was purified by column chromatography product was eluted in 10% ethyl acetate in n-hexane to obtain white solid (100%). ¹H NMR (400 MHz, CDCl₃) δ: 6.68-6.67 (t, 1H), 2.40-2.36 (d, 1H), 2.04-2.00 (m, 1H), 1.99-1.91 (m, 1H), 1.75-1.72 (m, 7H), 1.27 (s, 12H), 1.02-1.00 (t, 6H).

### Step 7: Synthesis of 4-(1,5-diethyl-8-oxabicyclo[3.2.1]oct-2-en-3-yl)-1-(tetrahydro-2H-pyran-2-yl)-1H-pyrazole-3-carbaldehyde

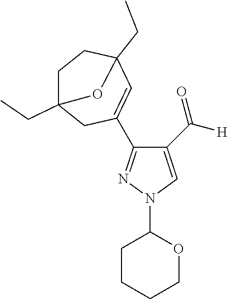 [SMILES: CCC12C=C(c3nn(C4CCCCO4)cc3C=O)CC(CC)(CC1)O2]

To a 30 ml sealed tube charged with 3-iodo-1-(tetrahydro-2H-pyran-2-yl)-1H-pyrazole-4-carbaldehyde (1.0 g, 3.26 mmol), was added 2-(1,5-diethyl-8-oxabicyclo[3.2.1]oct-2-en-3-yl)-4,4,5,5-tetramethyl-1,3,2-dioxaborolane (1.24 g, 4.2 mmol) and potassium phosphate (2.01 g, 9.78 mmol) in 1,4-dioxane:water mixture (10:2, 10 ml) and purged with argon gas for 30 min. After degassing, Pd(dppf)Cl₂ (0.023 g, 0.32 mmol). The reaction mixture was heated at 80° C. for 48 h. After completion the reaction mixture was diluted with ethyl acetate and filtered over Celite, washed with water (50 ml) and extracted with ethyl acetate (3×150 ml). The combined organic layers were dried over anhydrous sodium sulfate and concentrated. The crude compound was purified by column chromatography. The expected product was eluted in 20% ethyl acetate in n-hexane to obtain the title compound as a colorless liquid (62.7%). LCMS: m/z=345.5 [M+1].

### Step-8: Synthesis of 3-(1,5-diethyl-8-oxabicyclo[3.2.1]oct-2-en-3-yl)-1H-pyrazole-4-carbaldehyde

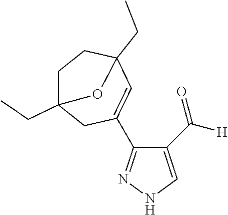 [SMILES: CCC12C=C(c3n[nH]cc3C=O)CC(CC)(CC1)O2]

To a stirred solution of 4-(1,5-diethyl-8-oxabicyclo[3.2.1]oct-2-en-3-yl)-1-(tetrahydro-2H-pyran-2-yl)-1H-pyrazole 3-carbaldehyde (4.41 g, 4.09 mmol) in THF (10 ml) was added with acetic acid:water (1:1, 20 ml) and heated to 80° C. for 12 h. The reaction was basified with sodium bicarbonate and extracted with ethyl acetate (3×100 ml). The combined organic layers were dried over anhydrous sodium sulfate and concentrated to obtain colorless liquid. The crude material was purified by column chromatography. The product was eluted in 50% ethyl acetate in n-hexane, concentrated to obtain colorless liquid (88.6%). LCMS: m/z=261.1 [M+1].

### Step-9: Synthesis of tert-butyl (2-(((3-(1,5-diethyl-8-oxabicyclo[3.2.1]oct-2-en-3-yl)-1H-pyrazol-4-yl)methyl)(methyl)amino)ethyl)(methyl)carbamate

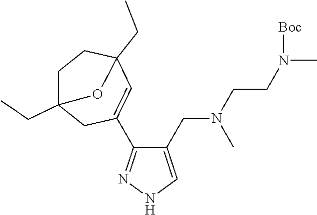 [SMILES: CCC12C=C(c3n[nH]cc3CN(C)CCN(C)C(=O)OC(C)(C)C)CC(CC)(CC1)O2]

To a stirred solution of 3-(1,5-diethyl-8-oxabicyclo[3.2.1]oct-2-en-3-yl)-1H-pyrazole-4-carbaldehyde (0.94 g, 3.61 mmol) in ethylene dichloride (10 ml) was added with tert-butyl (2-(dimethylamino)ethyl) (methyl)carbamate (1.01 g, 5.42 mmol) and stirred it for 30 min at room temperature. This was added with sodium triacetoxy borohydride (2.29 g, 10.8 mmol) portionwise and stirred it for 1 h. Reaction mixture was basified with sodium bicarbonate until basic and extracted with DCM (3×50 ml). The combined organic layer was dried over anhydrous sodium sulphate and concentrated to obtain yellow sticky liquid product (92.9%). LCMS: m/z=433.7 [M+1].

### Step-10: tert-butyl (2-(((3-((1R,3r,5S)-1,5-diethyl-8-oxabicyclo[3.2.1]octan-3-yl)-1H-pyrazol-4-yl)methyl)(methyl)amino)ethyl)(methyl)carbamate and tert-butyl (2-(((3-((1R,3s,5S)-1,5-diethyl-8-oxabicyclo[3.2.1]octan-3-yl)-1H-pyrazol-4-yl)methyl)(methyl)amino)ethyl)(methyl)carbamate

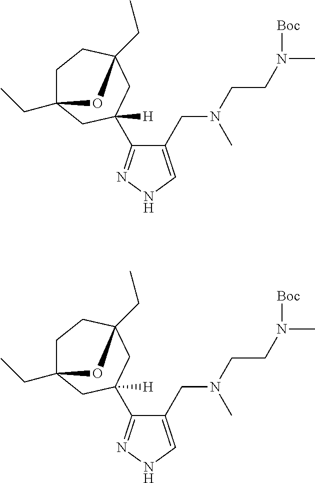

To the stirred solution of tert-butyl (2-(((3-(1,5-diethyl-8-oxabicyclo[3.2.1]oct-2-en-3-yl)-1H-pyrazol-4-yl)methyl)(methyl)amino)ethyl)(methyl)carbamate (0.66 g, 3.35 mmol) in THF (10 ml) was added 20% Palladium hydroxide on charcoal (600 mg) followed by stirring under H₂ at 1 atm pressure. After completion the reaction mixture was filtered over Celite and washed with methanol-DCM mixture (2×100 ml). The combined organic layer was dried under vacuum to obtain yellow liquid. This was purified over neutral alumina to give the title compound as a mixture of regioisomers (0.4 g). The isomers were separated by prep-HPLC using X Bridge C18 column (250×19 mm) and 0.1% NH₃ in water, 100% acetonitrile mobile phase to afford two fractions: Fraction 1 (35 mg), Fraction 2 (50 mg). LCMS: m/z=435.9 [M+1].

### Step-11: Synthesis of N1-((3-((1R,3r,5S)-1,5-diethyl-8-oxabicyclo[3.2.1]octan-3-yl)-1H-pyrazol-4-yl)methyl)-N1,N2-dimethylethane-1,2-diamine

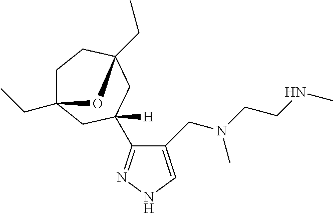 [SMILES: CC[C@]12CC[C@](CC)(C[C@H](c3n[nH]cc3CN(C)CCNC)C1)O2]

A solution of tert-butyl (2-(((3-((1R,3r,5S)-1,5-diethyl-8-oxabicyclo[3.2.1]octan-3-yl)-1H-pyrazol-4-yl)methyl)(methyl)amino)ethyl)(methyl)carbamate (35 mg, 0.080 mmol) in 4M HCl in isopropanol (2 ml) was stirred in a sealed tube for 24 h at rt. The reaction mixture was concentrated under vacuum to obtain a pale yellow solid residue which was triturated with n-pentane and diethyl ether to afford the dihydrochloride salt of the title compound as an off-white solid (100%). LCMS: m/z=335.58 [M+1]. ¹H-NMR (400 MHz, D₂O) δ: 7.67 (s, 1H), 4.24 (s, 2H), 3.42 (s, 4H), 3.22-3.18 (m, 1H), 2.71 (s, 3H), 2.68 (s, 3H), 1.87-1.85 (d, 2H), 1.74-1.68 (m, 4H), 1.54-1.49 (m, 6H), 0.79-0.75 (t, 6H).

### Step-12: Synthesis of N1-((3-((1R,3s,5S)-1,5-diethyl-8-oxabicyclo[3.2.1]octan-3-yl)-1H-pyrazol-4-yl)methyl)-N1,N2-dimethylethane-1,2-diamine

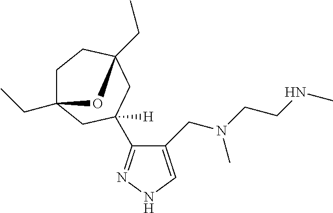 [SMILES: CC[C@]12CC[C@](CC)(C[C@@H](c3n[nH]cc3CN(C)CCNC)C1)O2]

A solution of tert-butyl (2-(((3-((1R,3s,5S)-1,5-diethyl-8-oxabicyclo[3.2.1]octan-3-yl)-1H-pyrazol-4-yl)methyl)(methyl)amino)ethyl)(methyl)carbamate (50 mg, 0.115 mmol) in 4M HCl in isopropanol (3 ml) was stirred in a sealed tube for 24 h at rt. The reaction mixture was concentrated under vacuum to obtain a pale yellow solid residue which was triturated with n-pentane and diethyl ether to afford the dihydrochloride salt of the title compound as an off-white solid (100%). LCMS: m/z=335.58 [M+1]. ¹H-NMR (400 MHz, D₂O) δ: 7.67 (s, 1H), 4.21 (s, 2H), 3.40 (s, 4H), 2.93-2.89 (m, 1H), 2.72 (s, 3H), 2.65 (s, 3H), 1.96-1.91 (m, 2H), 1.71-1.45 (m, 10H), 0.78-0.73 (t, 6H).

### Example B-19. Synthesis of N¹-((3-(4,4-bis(propoxymethyl)cyclohexyl)-1H-pyrazol-4-yl)methyl)-N¹,N²-dimethylethane-1,2-diamine (Compound 36)

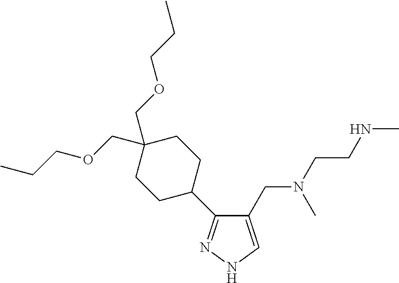 [SMILES: CCCOCC1(COCCC)CCC(c2n[nH]cc2CN(C)CCNC)CC1]

### Step-1: Synthesis of 8,8-bis(propoxymethyl)-1,4-dioxaspiro[4.5]decane

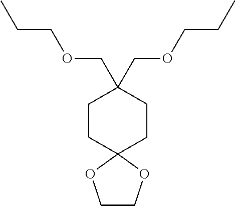 [SMILES: CCCOCC1(COCCC)CCC2(CC1)OCCO2]

A 60% suspension of NaH in mineral oil (2.40 g, 59.40 mmol) was washed with hexane (15 ml) 3-4 times and dried thoroughly under nitrogen. To this was added DMF (15 ml) and the mixture was stirred and cooled to 0-3° C. in an ice bath under nitrogen. To the above stirred suspension was added a solution of (1, 4-dioxaspiro[4.5]decane-8,8-diyl) dimethanol (3.0 g, 14.85 mmol) in DMF (35 ml) dropwise. Stirring was continued for 20 min. at 0° C. and then for 45 min at room temperature. N-Propyl iodide (5.8 ml, 59.40 mmol) was added dropwise at 0° C. The resulting mixture was stirred at 70° C. for 16 h. To the reaction mixture was added saturated NH₄Cl solution (20 mL) at 0-5° C. and water (80 ml) and extracted with ethyl acetate (3×60 ml). The organic extracts were combined, washed with brine (60 ml), dried over anhydrous Na₂SO₄, filtered and evaporated under reduced pressure to give a yellow liquid. The crude product was purified by silica gel chromatography under gravity using a mixture of ethyl acetate and hexane to afford the title compound as pale yellow liquid (34.7%). ¹H NMR (400 MHz, DMSO-d6) δ: 3.94 (s, 4H), 3.35 (t, J=6.4 Hz, 4H), 3.34 (s, 4H), 1.62 (m, 4H), 1.56 (m, 4H), 1.51 (sextet, J=7.6 Hz, 4H), 0.87 (t, J=7.2 Hz, 6H).

### Step-2: Synthesis of 4,4-bis(propoxymethyl)cyclohexan-1-one

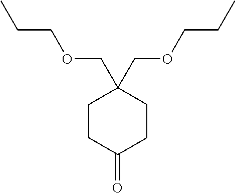 [SMILES: CCCOCC1(COCCC)CCC(=O)CC1]

To a stirred solution of 8,8-bis(propoxymethyl)-1,4-dioxaspiro[4.5]decane (1.58 g, 5.52 mmol) in DCM (20 ml) was added ferric chloride hexahydrate (2.98 g, 11.05 mmol) batchwise at 0-5° C. The mixture was stirred at room temperature for 4 h. Saturated aq. NaHCO₃ solution (12 ml) was added to the reaction mixture and it was diluted with water (30 ml). The resulting suspension was extracted with DCM (3×25 ml). The organic extracts were combined, washed with water, dried over anhydrous Na₂SO₄, filtered and evaporated under reduced pressure to give a brown liquid. The crude product was purified by silica gel chromatography under gravity using a mixture of ethyl acetate and hexane to afford the title compound as pale yellow liquid (55.6%). ¹H NMR (400 MHz, DMSO-d₆) δ: 3.35 (t, J=6.4 Hz, 4H), 3.34 (s, 4H), 2.26 (t, J=6.8 Hz, 4H), 1.67 (t, J=7.2 Hz, 4H), 1.51 (sextate, J=7.6 Hz, 4H), 0.87 (t, J=7.2 Hz, 6H).

### Step-3: Synthesis of 4,4-bis(propoxymethyl)cyclohex-1-en-1-yl trifluoromethanesulfonate

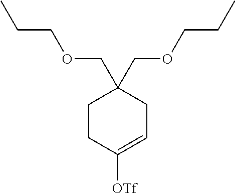 [SMILES: CCCOCC1(COCCC)CC=C(OS(=O)(=O)C(F)(F)F)CC1]

To a stirred solution of 4,4-bis(propoxymethyl)cyclohexan-1-one (500 mg, 2.06 mmol) in dried THF (10 ml) cooled at −60° C. in a dry ice:acetone bath was added LiHMDS (2.3 ml, 1M solution in THF, 2.27 mmol) dropwise under nitrogen. Stirring was continued at −60° C. for 1.5 h. A solution of 1,1,1-trifluoro-N-phenyl-N-((trifluoromethyl)sulfonyl)methanesulfonamide (812 mg, 2.27 mmol) in dried THF (8 ml) was added dropwise. The reaction mixture was allowed to warm to room temperature and stirred for 1.5 h. Sat. ammonium chloride solution (10 ml) was added and the mixture was concentrated to dryness. Water (60 ml) was added to the residue and the mixture was extracted with ethyl acetate (3×30 ml). The organic extracts were combined, washed with water (50 ml), dried over anhydrous Na₂SO₄, filtered and evaporated under reduced pressure to obtain a yellow liquid. The crude product was purified by silica gel chromatography under gravity using a mixture of ethyl acetate and hexane to afford the title compound as pale yellow liquid (79.1%). ¹H NMR (400 MHz, CDCl₃) δ: 5.68 (t, J=4.4 Hz, 1H), 3.36 (t, J=6.4 Hz, 4H), 3.27 (q, J=8.8 Hz, 4H), 2.33-2.29 (m, 2H), 2.13-2.09 (m, 2H), 1.73 (t, J=6.4 Hz, 2H), 1.56 (sextet, J=7.2 Hz, 4H), 0.91 (t, J=7.6 Hz, 6H).

### Step-4: Synthesis of 2-(4,4-bis(propoxymethyl)cyclohex-1-en-1-yl)-4,4,5,5-tetramethyl-1,3,2-dioxaborolane

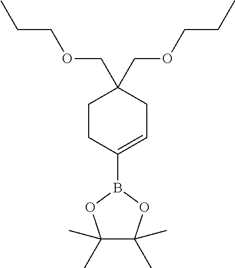 [SMILES: CCCOCC1(COCCC)CC=C(B2OC(C)(C)C(C)(C)O2)CC1]

To a stirred mixture of 4,4-bis(propoxymethyl)cyclohex-1-en-1-yl trifluoromethanesulfonate (600 mg, 1.60 mmol); 4,4,4′,4′,5,5,5′,5′-octamethyl-2,2′-bi(1,3,2-dioxaborolane) (406 mg, 1.60 mmol) and potassium acetate (470 mg, 4.80 mmol) in 1,4-dioxan (5 ml) purged and maintained with an inert atmosphere of argon was added [1,1′-Bis(diphenylphosphino) ferrocene]dichloropalladium(II) (117 mg, 0.160 mmol) and heated at 65° C. for 1 h. The mixture was concentrated under reduced pressure. Water (20 ml) was added into the brown viscous residue and the mixture was extracted with ethyl acetate (3×30 ml). The organic extracts were combined, dried over anhydrous Na₂SO₄, filtered and evaporated under reduced pressure to give a brown viscous mass which was purified by silica gel chromatography under gravity using a mixture of ethyl acetate and hexane to afford the title compound as pale yellow liquid (71%). ¹H NMR (400 MHz, CDCl₃) δ: 6.52 (t, J=2.0 Hz, 1H), 3.36 (t, J=6.4 Hz, 4H), 3.25 (m, 4H), 2.13-2.10 (m, 2H), 2.05-1.99 (m, 2H), 1.60-1.50 (m, 6H), 1.28 (m, 12H), 0.91 (t, J=7.6 Hz, 6H).

### Step-5: Synthesis of tert-butyl (2-(((3-(4,4-bis(propoxymethyl)cyclohex-1-en-1-yl)-1-(tetrahydro-2H-pyran-2-yl)-1H-pyrazol-4-yl)methyl)(methyl)amino)ethyl) (methyl)carbamate

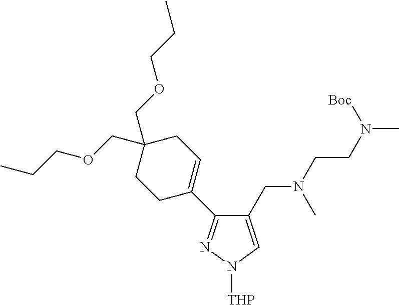

To a stirred mixture of 2-(4,4-bis(propoxymethyl)cyclohex-1-en-1-yl)-4,4,5,5-tetramethyl-1,3,2-dioxaborolane (300 mg, 0.926 mmol); tert-butyl (2-(((3-iodo-1-(tetrahydro-2H-pyran-2-yl)-1H-pyrazol-4-yl)methyl)amino)ethyl)(methyl)carbamate (340 mg, 0.712 mmol) and potassium phosphate (453 mg, 2.14 mmol) in 1,4-dioxan (2 ml) and water (0.8 ml) purged and maintained with an inert atmosphere of argon was added [1,1′-Bis(diphenylphosphino) ferrocene]dichloropalladium(II) (52 mg, 0.071 mmol) and heated at 75° C. for 50 minutes. The mixture was concentrated under reduced pressure. Water (30 ml) was added to the black oily residue and the mixture was extracted with ethyl acetate (3×20 ml). The organic extracts were combined, dried over anhydrous Na₂SO₄, filtered and evaporated under reduced pressure to give a black oily residue. The crude product was chromatographed over neutral alumina in a gravity column using a mixture of MeOH and DCM to afford the title compound as pale brown viscous mass (59.7%). LCMS: m/z=577.98 [M+1]. ¹H NMR (400 MHz, DMSO-d₆) δ: 7.67 (bs, 1H), 6.24-6.17 (m, 1H), 5.28-5.26 (m, 1H), 3.90 (bd, J=9.2, 1H), 3.60-3.58 (m, 1H), 3.32-3.22 (m, 10H), 2.72 (bd, J=16.8, 3H), 2.41-2.32 (m, 4H), 2.11 (bd, J=16.8, 3H), 2.02 (bs, 3H), 1.91-1.83 (m, 2H), 1.66-1.64 (m, 1H), 1.55-1.49 (m, 8H), 1.38-1.34 (m, 9H), 1.23-1.18 (m, 2H), 0.86 (t, J=6.8 Hz, 6H).

### Step-6: Synthesis of tert-butyl (2-(((3-(4,4-bis(propoxymethyl)cyclohexyl)-1-(tetrahydro-2H-pyran-2-yl)-1H-pyrazol-4-yl)methyl)(methyl)amino)ethyl)(methyl)carbamate

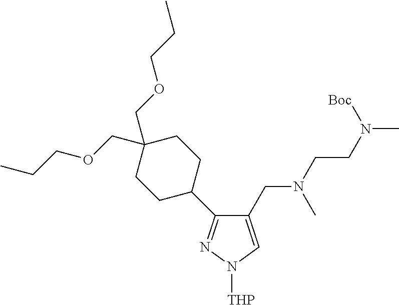

A solution of tert-butyl (2-(((3-(4,4-bis(propoxymethyl)cyclohex-1-en-1-yl)-1-(tetrahydro-2H-pyran-2-yl)-1H-pyrazol-4-yl)methyl)(methyl)amino)ethyl)(methyl)carbamate (230 mg, 0.40 mmol) in dry THF (10 ml) was hydrogenated under a pressure of 1 atm in the presence of palladium hydroxide (180 mg, 20% on charcoal) for 50 min at rt. The reaction mixture was filtered through Celite. The filtrate was evaporated to obtain a colorless viscous residue which was chromatographed over neutral alumina in a gravity column using a mixture of MeOH and DCM to afford the title compound as colorless viscous mass (64.9%). ¹H NMR (400 MHz, CDCl₃) δ: 7.44 (bs, 1H), 5.33-5.29 (m, 1H), 4.10-4.06 (m, 1H), 3.75-3.69 (m, 1H), 3.47 (s, 2H), 3.47-3.37 (m, 7H), 3.33-3.30 (m, 1H), 3.22 (s, 2H), 2.85 (s, 3H), 2.65-2.55 (m, 1H), 2.54-2.43 (m, 2H), 2.23 (s, 3H), 2.03 (bs 3H), 1.78-1.66 (m, 8H), 1.62-1.55 (m, 4H), 1.46 (s, 9H), 1.34-1.28 (m, 3H), 0.93 (t, J=7.6 Hz, 6H).

### Step-7: Synthesis of N¹-((3-(4,4-bis(propoxymethyl)cyclohexyl)-1H-pyrazol-4-yl)methyl)-N¹,N²-dimethylethane-1,2-diamine

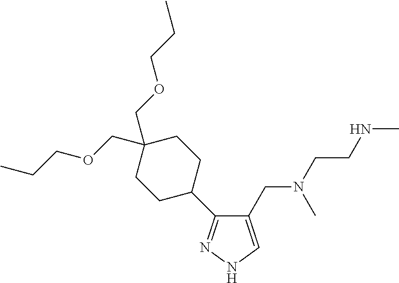 [SMILES: CCCOCC1(COCCC)CCC(c2n[nH]cc2CN(C)CCNC)CC1]

To a solution of tert-butyl (2-(((3-(4,4-bis(propoxymethyl)cyclohexyl)-1-(tetrahydro-2H-pyran-2-yl)-1H-pyrazol-4-yl)methyl)(methyl)amino)ethyl)(methyl)carbamate (150 mg, 0.260 mmol) in DCM (25 ml) and IPA (5 ml) was purged HCl gas for 3 h at room temperature. The reaction mixture was then stirred in sealed condition for 14 h. The mixture was concentrated under vacuum to obtain a yellow solid residue which was triturated with diethyl ether to afford the hydrochloride salt of the title as yellow powder (87.5%). LCMS: m/z=395.35 [M+1]. ¹H NMR (400 MHz, D₂O) δ: 7.78 (s, 1H), 4.30 (s, 2H), 3.50-3.16 (m, 10H), 3.22 (s, 2H), 2.75 (s, 3H), 2.71-2.69 (m, 1H), 2.70 (s, 3H), 1.62-1.57 (m, 6H), 1.52-1.45 (m, 4H), 1.30-1.20 (m, 2H), 0.79 (t, J=7.6 Hz, 6H).

### Example B-20. Synthesis of (4-(4-((methyl(2-(methylamino)ethyl)amino)methyl)-1H-pyrazol-3-yl)cyclohexane-1,1-diyl)dimethanol (Compound 44)

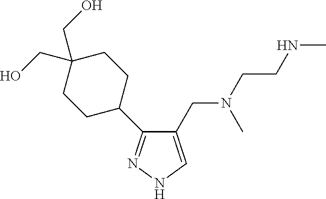 [SMILES: CNCCN(C)Cc1c[nH]nc1C1CCC(CO)(CO)CC1]

### Step 1: Synthesis of 8,8-bis((benzyloxy)methyl)-1,4-dioxaspiro[4.5]decane

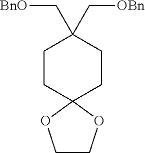 [SMILES: c1ccc(COCC2(COCc3ccccc3)CCC3(CC2)OCCO3)cc1]

A 60% suspension of NaH in mineral oil (3.16 g, 79.11 mmol) was washed with hexane (15 ml) 4-5 times and dried thoroughly under nitrogen. To this was added DMF (20 ml) and the mixture was stirred and cooled to 0-5° C. in an ice bath under nitrogen. To the above stirred suspension was added a solution of (1,4-dioxaspiro[4.5]decane-8,8-diyl)dimethanol (4.0 g, 19.78 mmol) in DMF (20 ml) dropwise. Stirring was continued for 45 min at room temperature. Benzyl bromide (13.53 g, 79.11 mmol) was added dropwise at 10-15° C. The resulting suspension was stirred at 75° C. for 16 h. To the reaction mixture was added water (150 ml) after cooling to room temperature and extracted with ethyl acetate (3×50 ml). The organic extracts were combined, washed with brine (50 ml) and water (50 ml), dried over anhydrous Na₂SO₄, filtered and evaporated under reduced pressure to give a yellow viscous residue. The crude product was purified by silica gel chromatography under gravity using a mixture of ethyl acetate and hexane to afford the title compound as pale yellow viscous liquid (63.5%). ¹H NMR (400 MHz, DMSO-d6) δ: 7.35-7.27 (m, 10H), 4.46 (s, 4H), 3.82 (s, 4H), 3.35 (s, 4H), 1.48 (s, 8H).

### Step-2: Synthesis of 4,4-bis((benzyloxy)methyl)cyclohexan-1-one

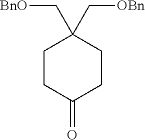 [SMILES: O=C1CCC(COCc2ccccc2)(COCc2ccccc2)CC1]

To a stirred solution of 8,8-bis((benzyloxy)methyl)-1,4-dioxaspiro[4.5]decane (2.50 g, 6.54 mmol) in DCM (30 ml) was added ferric chloride hexahydrate (3.53 g, 13.07 mmol) batch wise at 0-5° C. The mixture was stirred at room temperature for 4 h. The resulting suspension was filtered through Celite. The filtrate was evaporated to obtain a brown viscous liquid which was purified by silica gel chromatography under gravity using a mixture of ethyl acetate and hexane to afford the title compound as pale yellow viscous mass (90.5%). ¹H NMR (400 MHz, DMSO-d6) δ: 7.36-7.27 (m, 10H), 4.49 (s, 4H), 3.46 (s, 4H), 2.24 (t, J=6.8 Hz, 4H), 1.72 (t, J=7.2 Hz, 4H).

### Step-3: Synthesis of 4,4-bis((benzyloxy)methyl)cyclohex-1-en-1-yl trifluoromethanesulfonate

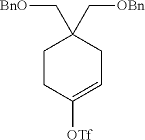 [SMILES: O=S(=O)(OC1=CCC(COCc2ccccc2)(COCc2ccccc2)CC1)C(F)(F)F]

To a stirred solution of 4,4-bis((benzyloxy)methyl)cyclohexan-1-one (1.80 g, 5.33 mmol) in dried THF (15 ml) cooled at −70° C. in a dry ice:acetone bath was added LiHMDS (5.9 ml, 1M solution in THF, 5.83 mmol) under nitrogen. Stirring was continued at −70° C. for 2 h. A solution of 1,1,1-trifluoro-N-phenyl-N-((trifluoromethyl)sulfonyl)methanesulfonamide (1.86 g, 5.22 mmol) in dried THF (8 ml) was added dropwise. The reaction mixture was allowed to warm to room temperature and stirred for 2 h, saturated ammonium chloride solution (5 ml) was added and the mixture was concentrated to dryness. Water (60 ml) was added to the residue and the mixture was extracted with ethyl acetate (4×30 ml). The organic extracts were combined, washed with water (50 ml), dried over anhydrous Na₂SO₄, filtered and evaporated under reduced pressure to give a pale brown viscous liquid. The crude product was purified by silica gel chromatography under gravity using a mixture of ethyl acetate and hexane to afford the title compound as pale yellow liquid (60%). ¹H NMR (400 MHz, CDCl₃) δ: 7.37-7.28 (m, 10H), 5.67 (t, J=4.0 Hz, 1H), 4.51 (s, 4H), 3.42 (d, J=8.8 Hz, 2H), 3.37 (d, J=8.8 Hz, 2H), 2.31-2.29 (m, 2H), 2.16-2.14 (m, 2H) 1.79 (t, J=6.4 Hz, 2H).

### Step-4: Synthesis of 2-(4,4-bis((benzyloxy)methyl)cyclohex-1-en-1-yl)-4,4,5,5-tetramethyl-1,3,2-dioxaborolane

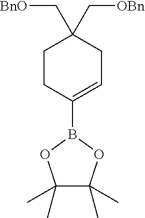 [SMILES: CC1(C)OB(C2=CCC(COCc3ccccc3)(COCc3ccccc3)CC2)OC1(C)C]

To a stirred mixture of 4,4-bis((benzyloxy)methyl)cyclohex-1-en-1-yl trifluoromethanesulfonate (1.5 g, 3.19 mmol); 4,4,4′,4′,5,5,5′,5′-octamethyl-2,2′-bi(1,3,2-dioxaborolane) (810 mg, 3.19 mmol) and potassium acetate (938 mg, 9.57 mmol) in 1,4-dioxan (10 ml) purged and maintained with an inert atmosphere of argon was added [1,1′-Bis(diphenylphosphino) ferrocene]dichloropalladium(II) (233 mg, 0.319 mmol) and heated at 65° C. for 1 h. The mixture was concentrated under reduced pressure. Water (30 ml) was poured into the brown viscous residue and the mixture was extracted with ethyl acetate (3×30 ml). The organic extracts were combined, dried over anhydrous Na₂SO₄, filtered and evaporated under reduced pressure to give a brown viscous mass which was purified by silica gel chromatography under gravity using a mixture of ethyl acetate and hexane to afford the title compound as pale yellow viscous mass (70%). ¹H NMR (400 MHz, CDCl₃) δ: 7.36-7.25 (m, 10H), 6.51 (brs, 1H), 4.50 (s, 4H), 3.42 (d, J=8.8 Hz, 2H), 3.36 (d, J=8.8 Hz, 2H), 2.13-2.10 (m, 2H), 2.07-2.05 (m, 2H), 1.60-1.57 (m, 2H), 1.32 (s, 12H).

### Step-5: Synthesis of tert-butyl (2-(((3-(4,4-bis((benzyloxy)methyl)cyclohex-1-en-1-yl)-1-(tetrahydro-2H-pyran-2-yl)-1H-pyrazol-4-yl)methyl)(methyl)amino)ethyl)(methyl) carbamate

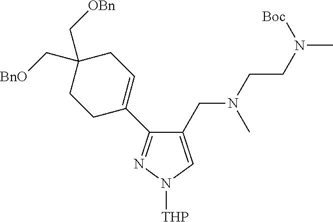

To a stirred mixture of 2-(4,4-bis((benzyloxy)methyl)cyclohex-1-en-1-yl)-4,4,5,5-tetramethyl-1,3,2-dioxaborolane (900 mg, 2.0 mmol); tert-butyl (2-(((3-iodo-1-(tetrahydro-2H-pyran-2-yl)-1H-pyrazol-4-yl)methyl)amino)ethyl)(methyl)carbamate (739 mg, 1.5 mmol) and potassium phosphate (1.27 g, 6.0 mmol) in 1,4-dioxan (5 ml) and water (1.5 ml) purged and maintained with an inert atmosphere of argon was added [1,1′-Bis(diphenylphosphino) ferrocene]dichloropalladium(II) (110 mg, 0.150 mmol) and heated at 70-75° C. for 4.5 h. The mixture was concentrated under reduced pressure. Water (30 ml) was added to the brown viscous residue and the mixture was extracted with ethyl acetate (3×30 ml). The organic extracts were combined, dried over anhydrous Na₂SO₄, filtered and evaporated under reduced pressure to give a brown viscous mass. The crude product was chromatographed over neutral alumina in a gravity column using a mixture of MeOH and DCM to afford the title compound as pale yellow viscous mass (41%). ¹H NMR (400 MHz, DMSO-d₆) δ: 7.66 (s, 1H), 7.35-7.24 (m, 10H), 6.24-6.18 (m, 1H), 5.27 (dd, J₁=2.0 Hz, J₂=10.0 Hz, 1H), 4.47 (s, 4H), 3.90 (brd, J=11.6 Hz, 1H), 3.62-3.56 (m, 1H), 3.40-3.33 (m, 4H), 3.25 (brs, 4H), 2.74-2.69 (m, 3H), 2.40 (t, J=6.0 Hz, 2H), 2.34 (brs, 2H), 2.13-2.00 (m, 6H), 1.93-1.90 (m, 1H), 1.86-1.83 (m, 1H), 1.63-1.60 (m, 3H), 1.52-1.50 (m, 2H), 1.33 (brs, 9H).

### Step-6: Synthesis of tert-butyl (2-(((3-(4,4-bis((benzyloxy)methyl)cyclohexyl)-1-(tetrahydro-2H-pyran-2-yl)-1H-pyrazol-4-yl)methyl)(methyl)amino)ethyl)(methyl)carbamate

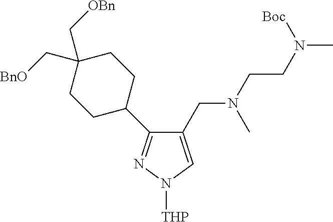

A solution of tert-butyl (2-(((3-(4,4-bis((benzyloxy)methyl)cyclohex-1-en-1-yl)-1-(tetrahydro-2H-pyran-2-yl)-1H-pyrazol-4-yl)methyl)(methyl)amino)ethyl)(methyl)carbamate (300 mg, 0.440 mmol) in dry THF (10 ml) was hydrogenated under a pressure of 1 atm in the presence of palladium hydroxide (250 mg, 20% on charcoal) for 50 min at rt. The reaction mixture was filtered through Celite. The filtrate was evaporated to obtain a colorless viscous residue which was chromatographed over neutral alumina in a gravity column using a mixture of MeOH and DCM to afford the title compound as colorless viscous mass (86.4%). ¹H NMR (400 MHz, CDCl₃) δ: 7.44 (s, 1H), 7.35-7.24 (m, 10H), 5.31-5.29 (m, 1H), 4.53 (s, 4H), 4.08 (d, J=11.2 Hz, 1H), 3.72-3.67 (m, 1H), 3.62 (s, 2H), 3.35 (s, 4H), 3.26-3.30 (m, 2H), 2.84 (s, 3H), 2.63-2.60 (m, 1H), 2.47-2.51 (m, 2H), 2.21 (s, 3H), 2.07-1.90 (m, 3H), 1.89-1.85 (m, 3H), 1.75-1.59 (m, 6H), 1.45 (s, 9H), 1.38-1.34 (m, 2H).

### Step-7: Synthesis of (4-(4-((methyl(2-(methylamino)ethyl)amino)methyl)-1H-pyrazol-3-yl)cyclohexane-1,1-diyl)dimethanol

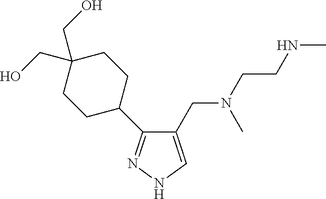 [SMILES: CNCCN(C)Cc1c[nH]nc1C1CCC(CO)(CO)CC1]

A solution of tert-butyl (2-(((3-(4,4-bis((benzyloxy)methyl)cyclohexyl)-1-(tetrahydro-2H-pyran-2-yl)-1H-pyrazol-4-yl)methyl)(methyl)amino)ethyl)(methyl)carbamate (260 mg, 0.380 mmol) in HBr (0.6 ml, 48% in acetic acid) was stirred for 4 h at room temperature. The reaction was concentrated under vacuum to obtain a red viscous liquid which was purified by prep-HPLC with Phenomenex Luna C8 column (250×21.2 mm), 0.1% TFA in water and 100% acetonitrile mobile phase to afford the TFA salt of the title compound (21.4%). LCMS: m/z=311.25 [M+1]. ¹H NMR (400 MHz, MeOD) δ: 7.74 (s, 1H), 4.35 (s, 2H), 3.74 (s, 2H), 3.53 (s, 4H), 3.42 (s, 2H), 3.34-3.32 (m, 1H), 2.87 (s, 3H), 2.79 (s, 3H), 1.81-1.70 (m, 6H), 1.43-1.31 (m, 2H).

### Example B-21. Synthesis of N1-((3-(4,4-bis((2-ethoxyethoxy)methyl)cyclohexyl)-1H-pyrazol-4-yl)methyl)-N1,N2-dimethylethane-1,2-diamine (Compound 39)

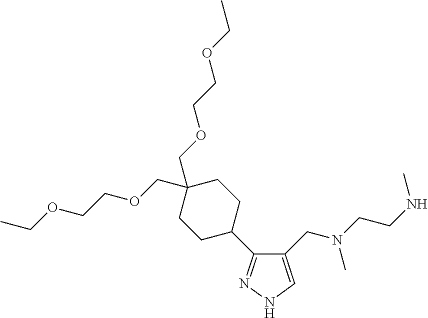 [SMILES: CCOCCOCC1(COCCOCC)CCC(c2n[nH]cc2CN(C)CCNC)CC1]

### Step-1: Synthesis of 8,8-bis((2-ethoxyethoxy)methyl)-1,4-dioxaspiro[4.5]decane

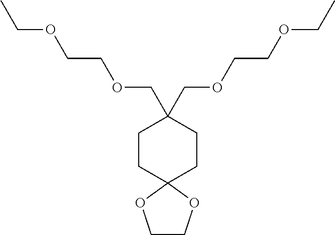 [SMILES: CCOCCOCC1(COCCOCC)CCC2(CC1)OCCO2]

To a suspension of NaH (washed with n-hexane, 1.57 g, 65.34 mmol) in DMF (10 ml) under an inert atmosphere at 0-5° C. temperature, solution of (1,4-dioxaspiro[4.5]decane-8,8-diyl)dimethanol (3.3 g, 16.37 mmol) in DMF (15 ml) was added slowly. Resulting suspension was stirred for 45 min at cooling condition; 1-bromo-2-ethoxyethane (10.0 g, 65.34 mmol) was added dropwise. After addition, resulting clear solution was heated at 70-75° C. for 16 h. To this solution, ice cold water (30 ml) was added slowly and extracted with diethyl ether (3×50 ml). The combined organic layer was washed with cold brine solution and concentrated under vacuum to obtain the crude product which was purified by gravity column chromatography eluting with 15% ethyl acetate in hexane to afford the title compound (13.4%) ¹H NMR (400 MHz, CDCl₃) δ: 3.95 (s, 4H), 3.65-3.52 (m, 12H), 3.36 (s, 4H), 1.64-1.61 (m, 4H), 1.58-1.51 (m, 4H), 1.28-1.27 (d, J=2.8 Hz, 6H).

### Step-2: Synthesis of 4,4-bis((2-ethoxyethoxy)methyl)cyclohexan-1-one

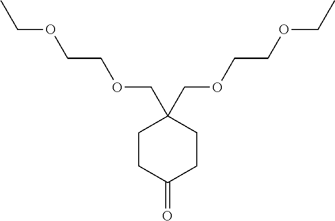 [SMILES: CCOCCOCC1(COCCOCC)CCC(=O)CC1]

To a stirred solution of 8,8-bis((2-ethoxyethoxy)methyl)-1,4-dioxaspiro[4.5]decane (1.2 g, 3.46 mmol) in DCM (10 ml), ferric chloride hexahydrate (1.12 g, 6.92 mmol) was added portionwise. The resulting solution was stirred for 16 h. To the solution, sat. NaHCO₃ solution (25 ml) was added and extracted with DCM (3×25 ml), dried over Na₂SO₄ and concentrated to obtained crude product which was purified by gravity column chromatography. Title compound was eluted with 8% ethyl acetate in hexane to afford a pale yellow volatile liquid (86.9%). ¹H NMR (400 MHz, CDCl₃) δ: 3.67-3.58 (m, 8H), 3.56-3.49 (m, 4H), 3.46 (s, 4H), 2.39 (s, 4H), 1.84-1.80 (m, 4H), 1.26 (s, 6H).

### Step-3: Synthesis of 4,4-bis((2-ethoxyethoxy)methyl)cyclohex-1-en-1-yl trifluoromethanesulfonate

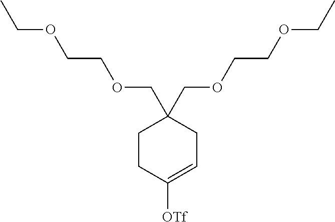 [SMILES: CCOCCOCC1(COCCOCC)CC=C(OS(=O)(=O)C(F)(F)F)CC1]

To a stirred solution of 4,4-bis((2-ethoxyethoxy)methyl)cyclohexan-1-one (0.91 g, 2.98 mmol) in THF (10 ml) in an inert condition at −70° C., LiHMDS (0.597 g, 3.57 ml, 3.57 mmol) was added slowly. Resultant solution was stirred for 1 h at same condition. Finally, a solution of N-phenyl-O-((trifluoromethyl)sulfonyl)-N-(((trifluoromethyl)sulfonyl)oxy)hydroxylamine (0.96 g, 2.68 mmol) in THF (5 ml) was added dropwise. The solution was stirred for 1 h at rt. Upon completion of the reaction, sat. ammonium chloride (25 ml) was added and extracted with diethyl ether (3×20 ml). The combined organic layers were dried over Na₂SO₄ and concentrated under reduced pressure to obtain crude product which was purified by gravity column chromatography. Product was eluted with 10% ethyl acetate in hexane as faint yellow volatile liquid (51.3%). ¹H NMR (400 MHz, CDCl₃) δ: 5.69 (s, 1H), 3.62-3.58 (m, 6H), 3.57-3.52 (m, 6H), 3.40-3.38 (d, J=9.2 Hz, 2H), 3.34-3.32 (d, J=9.2 Hz, 2H), 2.33 (s, 2H), 2.13-2.12 (d, J=4.0 Hz, 2H), 1.78-1.75 (m, 2H), 1.25-1.19 (m, 6H).

### Step-4: Synthesis of 2-(4,4-bis((2-ethoxyethoxy)methyl)cyclohex-1-en-1-yl)-4,4,5,5-tetramethyl-1,3,2-dioxaborolane

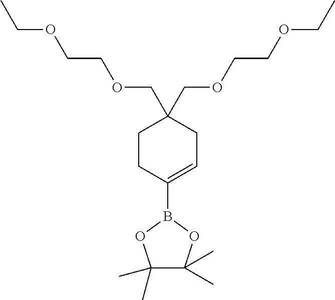 [SMILES: CCOCCOCC1(COCCOCC)CC=C(B2OC(C)(C)C(C)(C)O2)CC1]

In a 25 ml 3-necked round bottom flask, 4,4-bis((2-ethoxyethoxy)methyl)cyclohex-1-en-1-yl trifluoromethanesulfonate (0.66 g, 1.52 mmol), 1,4-dioxane (8 ml) was placed. The resulting solution was stirred under a static argon degassing for 20 min at which time potassium acetate (0.446 g, 4.56 mmol), 4,4,4′,4′,5,5,5′,5′-octamethyl-2,2′-bi(1,3,2-dioxaborolane) (0.425 g, 1.67 mmol) and 1,1′-bis(diphenylphosphino)ferrocene]dichloropalladium(II) (0.111 g, 0.15 mmol) were added at rt. After argon degassing for 10 min the resulting mixture was heated at 80° C. for 2 h. After heating, the resulting brown mass was concentrated and placed on silica gel bed for column chromatography which was eluting with 8% ethyl acetate in hexane as pale yellow oil (71.8%). ¹H NMR (400 MHz, CDCl₃) δ: 6.52 (s, 1H), 3.63-3.58 (m, 8H), 3.36-3.34 (d, J=8.8 Hz, 4H), 3.31-3.29 (d, J=9.2 Hz, 4H) 2.12-2.10 (m, 2H), 2.02-2.00 (m, 2H), 1.55-1.51 (m, 2H), 1.29 (s, 12H), 1.26-1.19 (m, 6H).

### Step-5: Synthesis of tert-butyl (2-(((3-(4,4-bis((2-ethoxyethoxy)methyl)cyclohex-1-en-1-yl)-1-(tetrahydro-2H-pyran-2-yl)-1H-pyrazol-4-yl)methyl)(methyl)amino)ethyl) (methyl)carbamate

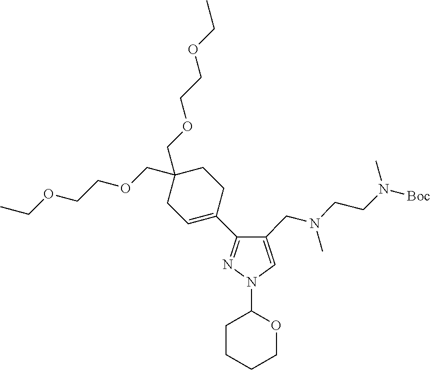 [SMILES: CCOCCOCC1(COCCOCC)CC=C(c2nn(C3CCCCO3)cc2CN(C)CCN(C)C(=O)OC(C)(C)C)CC1]

In a 25 ml 3-necked RBF, 2-(4,4-bis((2-ethoxyethoxy)methyl)cyclohex-1-en-1-yl)-4,4,5,5-tetramethyl-1,3,2-dioxaborolane (0.45 g, 1.09 mmol), 1,4-dioxane (5 ml) and water (1.5 ml). The resulting solution was stirred under argon for 30 min followed by addition potassium phosphate (0.695 g, 3.2 mmol), tert-butyl(2-(((3-iodo-1-(tetrahydro-2H-pyran-2-yl)-1H-pyrazol-4-yl)methyl)(methyl)amino)ethyl)(methyl)carbamate (0.438 g, 0.92 mmol) in to the resulting solution. Finally, Pd(dppf)Cl₂ (0.08 g, 0.11 mmol) was added. Again argon degassing was kept for 10 min and reaction mixture was heated at 70-75° C. for 2 h. The resulting brown mass was concentrated, water (20 ml) was added and extracted with ethyl acetate (2×25 ml), organic layer dried over Na₂SO₄. The crude material was purified by column chromatography to afford the title compound was eluted with 0.1% MeOH in DCM (61.9%). LCMS: m/z=637.84 [M+1].

### Step-6: Synthesis of tert-butyl (2-(((3-(4,4-bis((2-ethoxyethoxy)methyl)cyclohexyl)-1-(tetrahydro-2H-pyran-2-yl)-1H-pyrazol-4-yl)methyl)(methyl)amino)ethyl) (methyl)carbamate

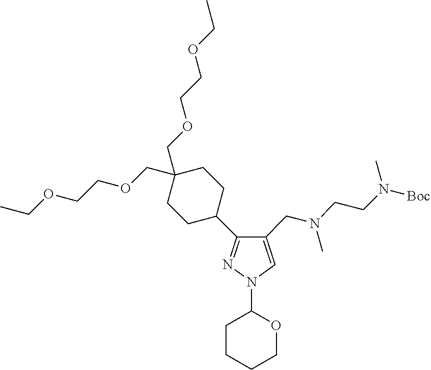 [SMILES: CCOCCOCC1(COCCOCC)CCC(c2nn(C3CCCCO3)cc2CN(C)CCN(C)C(=O)OC(C)(C)C)CC1]

To a stirred solution of tert-butyl (2-(((3-(4,4-bis((2-ethoxyethoxy)methyl)cyclohex-1-en-1-yl)-1-(tetrahydro-2H-pyran-2-yl)-1H-pyrazol-4-yl)methyl)(methyl)amino)ethyl)(methyl)carbamate (0.35 g, 1.09 mmol) in THF (7 ml) and MeOH (1 ml), Palladium hydroxide (0.25 g) was added and purged with H₂ for 45 min. After completion, the reaction mixture was diluted with 20% ethyl acetate in methanol (20 ml) and filtered through Celite. The Celite was washed with 20% ethyl acetate in methanol (2×10 ml). The filtrate was concentrated under reduced pressure to afford product (74%). LCMS: m/z=639.71 [M+1].

### Step-7: Synthesis of N1-((3-(4,4-bis((2-ethoxyethoxy)methyl)cyclohexyl)-1H-pyrazol-4-yl)methyl)-N1,N2-dimethylethane-1,2-diamine

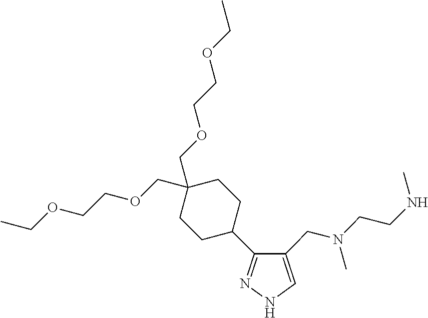 [SMILES: CCOCCOCC1(COCCOCC)CCC(c2n[nH]cc2CN(C)CCNC)CC1]

To a solution of tert-butyl (2-(((3-(4,4-bis((2-ethoxyethoxy)methyl)cyclohexyl)-1-(tetrahydro-2H-pyran-2-yl)-1H-pyrazol-4-yl)methyl)(methyl)amino)ethyl)(methyl)carbamate (0.1 g, 0.55 mmol) in DCM (5 ml) and IPA (2 ml). HCl gas passed through the solution. Completion of reaction was monitored through LCMS analysis. Evaporated solvents under reduced pressure to obtained colourless oil which was triturated using n-pentane and diethyl ether to afford off-white solid (73.1%). LCMS: m/z=455.11 [M+1]. ¹H NMR (400 MHz, D₂O) δ: 7.75 (s, 1H), 4.28 (s, 2H), 3.56-3.54 (m, 10H), 3.48-3.43 (m, 8H), 3.24 (s, 2H), 2.73 (s, 3H), 2.67 (s, 4H), 1.65-1.55 (m, 6H), 1.28-1.22 (m, 2H), 1.08-1.03 (m, 6H).

### Example B-22. Synthesis of N1-((3-(4,4-bis((cyclopropylmethoxy)methyl)cyclohexyl)-1H-pyrazol-4-yl)methyl)-N1,N2-dimethylethane-1,2-diamine (Compound 41)

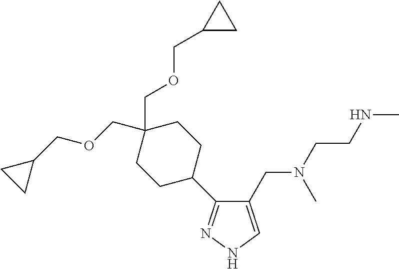 [SMILES: CNCCN(C)Cc1c[nH]nc1C1CCC(COCC2CC2)(COCC2CC2)CC1]

### Step-1: Synthesis of 8,8-bis((cyclopropylmethoxy)methyl)-1,4-dioxaspiro[4.5]decane

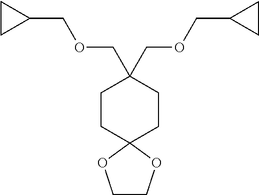 [SMILES: C1COC2(CCC(COCC3CC3)(COCC3CC3)CC2)O1]

To a stirred solution of (1,4-dioxaspiro[4.5]decane-8,8-diyl)dimethanol (5 g, 24.7 mmol) in DMF (60 ml) was added sodium hydride (3.93 g, 99 mmol) portion wise at 0° C. under nitrogen atmosphere. The mixture was stirred at rt for 30 min. (Bromomethyl)cyclopropane (13.36 g, 99 mmol) was then added dropwise. The mixture was stirred at room temperature for 15 h. To the reaction mixture was added water (150 ml) after cooling to room temperature and extracted with ethyl acetate (3×50 ml). The organic extracts were combined, washed with brine (50 ml) and water (50 ml), dried over anhydrous Na₂SO₄, evaporated to obtain a brown viscous liquid which was purified by silica gel column chromatography using a mixture of DCM and hexane to afford the title compound as pale yellow viscous mass (92.1%). ¹H NMR (400 MHz, CDCl₃) δ: 3.95 (s, 4H), 3.35 (s, 4H), 3.28 (d, J=6.4 Hz, 4H), 1.66-1.62 (m, 6H), 1.59-1.56 (m, 4H), 1.06-1.01 (m, 2H), 0.52 (m, 4H), 0.51 (m, 4H).

### Step-2: Synthesis of 4,4-bis((cyclopropylmethoxy)methyl)cyclohexan-1-one

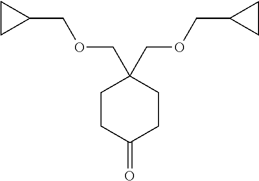 [SMILES: O=C1CCC(COCC2CC2)(COCC2CC2)CC1]

To a stirred solution of 8,8-bis((cyclopropylmethoxy)methyl)-1,4-dioxaspiro[4.5]decane (3.8 g, 12.5 mmol) in DCM (30 ml) was added ferric chloride hexahydrate (6.70 g, 25.0 mmol) portionwise at 0-5° C. The mixture was stirred at rt for 4 h. The resulting suspension was filtered through Celite. The filtrate was evaporated to obtain a brown viscous liquid which was purified by silica gel column chromatography using a mixture of ethyl acetate and hexane to afford the title compound as pale yellow viscous mass. Desired compound was confirmed by mass analysis (81.8%).

### Step-3: Synthesis of 4,4-bis((cyclopropylmethoxy)methyl)cyclohex-1-en-1-yl trifluoromethanesulfonate

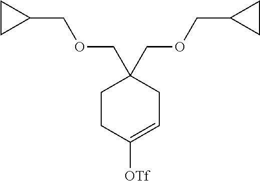 [SMILES: O=S(=O)(OC1=CCC(COCC2CC2)(COCC2CC2)CC1)C(F)(F)F]

To a stirred solution of 4,4-bis((cyclopropylmethoxy)methyl)cyclohexan-1-one (500 mg, 1.8 mmol) in dry THF (5.5 ml) cooled at −70° C. in a dry ice:acetone bath, was added LiHMDS (2.4 ml, 1M solution in THF, 2.4 mmol) under nitrogen atmosphere. Stirring was continued at −70° C. for 2 h. A solution of 1,1,1-trifluoro-N-phenyl-N-((trifluoromethyl)sulfonyl) methanesulfonamide (668 mg, 1.87 mmol) in dried THF (5 ml) was added dropwise. The reaction mixture was allowed to warm to room temperature and stirred for 2 h. Saturated ammonium chloride solution (5 ml) was then added and the mixture was concentrated to dryness. Water (30 ml) was added to the residue and the mixture was extracted with ethyl acetate (4×10 ml). The organic extracts were combined, washed with water (20 ml), dried over anhydrous Na₂SO₄, filtered and evaporated under reduced pressure to give a pale brown viscous liquid. The crude product was purified by silica gel column chromatography using a mixture of ethyl acetate and hexane to afford the title compound as pale yellow liquid (60%). ¹H NMR (400 MHz, CDCl₃) δ: 5.70 (d, 1H), 3.37-3.15 (m, 8H), 2.34-2.33 (m, 2H), 2.18 (m, 1H), 2.14-2.10 (m, 2H), 1.78-1.73 (m, 3H), 1.07-1.00 (m, 2H), 0.54-0.50 (m, 4H) 0.21-0.18 (m, 4H).

### Step-4: Synthesis of 2-(4,4-bis((cyclopropylmethoxy)methyl)cyclohex-1-en-1-yl)-4,4,5,5-tetramethyl-1,3,2-dioxaborolane

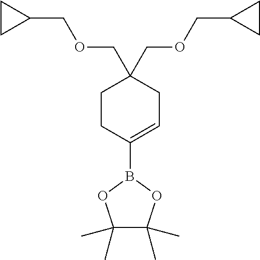 [SMILES: CC1(C)OB(C2=CCC(COCC3CC3)(COCC3CC3)CC2)OC1(C)C]

To a stirred mixture of 4,4-bis((cyclopropylmethoxy)methyl)cyclohex-1-en-1-yl trifluoromethanesulfonate (450 mg, 1.1 mmol); 4,4,4′,4′,5,5,5′,5′-octamethyl-2,2′-bi(1,3,2-dioxaborolane) (315.6 mg, 1.2 mmol) and potassium acetate (364 mg, 3.7 mmol) in 1,4-dioxan (3 ml) purged and maintained with an inert atmosphere of argon was added Pd(dppf)Cl₂ (83 mg, 0.11 mmol) and heated at 60-65° C. for 1 h. The mixture was concentrated under reduced pressure. Water (10 ml) was poured into the brown viscous residue and the mixture was extracted with ethyl acetate (3×10 ml). The organic extracts were combined, dried over anhydrous Na₂SO₄, filtered and evaporated under reduced pressure to give a brown viscous mass which was purified by silica gel column chromatography using a mixture of ethyl acetate and hexane to afford the title compound as pale yellow viscous mass (70%). ¹H NMR (400 MHz, CDCl₃) δ: 6.52 (s, 1H), 3.34-3.18 (m, 8H), 2.18 (m, 2H), 2.12 (m, 2H), 1.57 (m, 2H), 1.55-1.48 (m, 2H), 1.06-1.01 (m, 2H), 0.52-0.47 (m, 4H) 0.21 (m, 4H).

### Step-5: Synthesis of tert-butyl (2-(((3-(4,4-bis((cyclopropylmethoxy)methyl)cyclohex-1-en-1-yl)-1-(tetrahydro-2H-pyran-2-yl)-1H-pyrazol-4-yl)methyl)(methyl)amino)ethyl)(methyl)carbamate

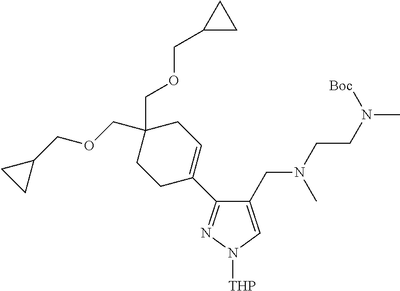

To a stirred mixture of 2-(4,4-bis((cyclopropylmethoxy)methyl)cyclohex-1-en-1-yl)-4,4,5,5-tetramethyl-1,3,2-dioxaborolane (100 mg, 0.26 mmol); tert-butyl (2-(((3-iodo-1-(tetrahydro-2H-pyran-2-yl)-1H-pyrazol-4-yl)methyl)amino)ethyl)(methyl)carbamate (115 mg, 0.23 mmol) and tripotassium phosphate (170 mg, 0.79 mmol) in 1,4-dioxan (1.5 ml) and water (0.5 ml) purged and maintained with an inert atmosphere of argon was added Pd(dppf)Cl₂ (18 mg, 0.025 mmol) and heated at 70-75° C. for 4.5 h. The mixture was concentrated under reduced pressure. Water (10 ml) was added to the brown viscous residue and the mixture was extracted with ethyl acetate (3×10 ml). The organic extracts were combined, dried over anhydrous Na₂SO₄, filtered and evaporated under reduced pressure to give a brown viscous mass. The crude product was chromatographed over neutral alumina in a gravity column using a mixture of ethyl acetate and hexane to afford the title compound as pale yellow viscous mass (57%). LCMS: m/z=601 [M+1].

### Step-6: Synthesis of tert-butyl (2-(((3-(4,4-bis((cyclopropylmethoxy)methyl)cyclohexyl)-1-(tetrahydro-2H-pyran-2-yl)-1H-pyrazol-4-yl)methyl)(methyl)amino)ethyl)(methyl)carbamate

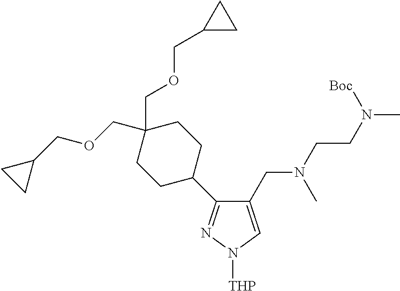

A solution of tert-butyl (2-(((3-(4,4-bis((cyclopropylmethoxy)methyl)cyclohex-1-en-1-yl)-1-(tetrahydro-2H-pyran-2-yl)-1H-pyrazol-4-yl)methyl)(methyl)amino)ethyl)(methyl)carbamate (150 mg, 0.24 mmol) in dry THF (20 ml) was hydrogenated under a pressure of 1 atm in the presence of palladium hydroxide (145 mg, 20% on charcoal) for 50 min at rt. The reaction mixture was filtered through Celite. The filtrate was evaporated to obtain a colorless viscous residue which was chromatographed over neutral alumina in a gravity column using a mixture of MeOH and DCM to afford the title compound as colorless viscous mass (56%). LCMS: m/z=604 [M+1].

### Step 7: Synthesis of N1-((3-(4,4-bis((cyclopropylmethoxy)methyl)cyclohexyl)-1H-pyrazol-4-yl)methyl)-N1,N2-dimethylethane-1,2-diamine

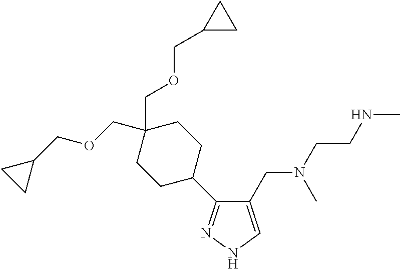 [SMILES: CNCCN(C)Cc1c[nH]nc1C1CCC(COCC2CC2)(COCC2CC2)CC1]

To a stirred solution of tert-butyl (2-(((3-(2,2-dimethyl-1-oxaspiro[4.5]decan-8-yl)-1H-pyrazol-4-yl)methyl)(methyl)amino)ethyl)(methyl)carbamate (0.15 g, 0.34 mmol) in DCM (3 ml) was added TFA (0.3 ml) at 0° C. The resulting solution was stirred at room temperature for 1 h and concentrated under reduced pressure to obtain 35 mg compound which was triturated with n-pentane to afford N1-((3-((5r,8r)-2,2-dimethyl-1-oxaspiro[4.5]decan-8-yl)-1H-pyrazol-4-yl)methyl)-N1,N2-dimethylethane-1,2-diamine (33%). LCMS: m/z=419 [M+1]. ¹H-NMR (400 MHz, D2O) δ: 7.70 (s, 1H), 4.27 (s, 2H), 3.55-3.43 (m, 6H), 3.26-3.22 (m, 6H), 2.73 (s, 3H), 2.68 (s, 3H), 1.64-1.56 (m, 4H), 1.25 (m, 2H), 0.95 (m, 2H), 0.44 (m, 4H), 0.10 (brs, 4H).

### Example B-23. Synthesis of N1-((3-((5S,8S)-3,3-dimethyl-1-oxaspiro[4.5]decan-8-yl)-1H-pyrazol-4-yl)methyl)-N1,N2,N2-trimethylethane-1,2-diamine and N1-((3-((5r,8r)-3,3-dimethyl-1-oxaspiro[4.5]decan-8-yl)-1H-pyrazol-4-yl)methyl)-N1,N2,N2-trimethylethane-1,2-diamine. (Compound 42 and 43)

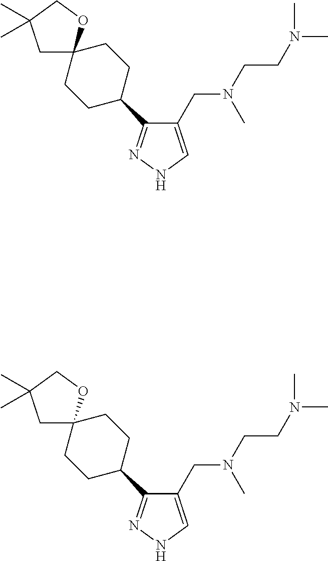

### Step-1: Synthesis of 3-(benzyloxy)-2,2-dimethylpropan-1-ol

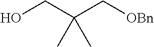

Into a 10-L 4-neck round-bottom flask, was placed 2,2-dimethylpropane-1,3-diol (200 g, 1920 mmol), toluene (1 L), 50% KOH (aq. solution, IL), n-Bu₄NI (36 g, 97 mmol). Then (bromomethyl)benzene (328 g, 1920 mmol, 1.00 equiv.) was added at 0° C. The resulting solution was stirred at room temperature for 16 h. The reaction was then quenched by the addition of 1 L of ice-water. The resulting solution was extracted with 2×4 L of ethyl acetate. The combined organic layer was washed with 2×4 L of brine, dried over anhydrous sodium sulfate and concentrated under vacuum. The crude product was purified by silica gel column chromatography to afford the title compound as light yellow oil (77%). LCMS: m/z=195.0 [M+1]. ¹H-NMR (300 MHz, CDCl₃) δ: 7.37-7.25 (m, 5H), 4.47 (m, 3H), 3.18 (d, J=6.4 Hz, 4H), 0.82 (s, 6H).

### Step-2: Synthesis of 1-((3-iodo-2,2-dimethylpropoxy)methyl) benzene

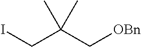

Into a 10 L 4-necked round-bottom flask, was placed 3-(benzyloxy)-2, 2-dimethylpropan-1-ol (150 g, 773 mmol), imidazole (103 g, 1540 mmol), PPh₃ (302 g, 1159 mmol), tetrahydrofuran (10 L). I₂ (293 g, 1159 mmol) was added slowly at 0° C. The resulting solution was stirred at rt for 16 h. The reaction was heated to 80° C. for 4 h. The reaction mixture was then cooled to rt and quenched by the addition of 300 ml of ice water. The resulting mixture was concentrated under vacuum and then extracted with 2×5 L of ethyl acetate. The combined organic layers were washed with 3×2 L of Na₂SO₃ (sat. aq.) and concentrated under vacuum. The residue was applied onto a silica gel column with ethyl acetate/hexane (1:10) as mobile phase. The collected fractions were combined and concentrated under vacuum to give 220 g of the title compound as colorless oil (93.5%). ¹H-NMR (300 MHz, CDCl₃) δ: 7.36-7.25 (m, 5H), 4.49 (s, 2H), 3.30 (d, J=6.0 Hz, 2H), 3.24 (s, J=6.0 Hz, 2H) 1.07 (s, 6H).

### Step-3: Synthesis of 8-[3-(benzyloxy)-2,2-dimethylpropyl]-1,4-dioxaspiro[4.5]decan-8-ol

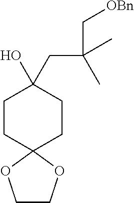 [SMILES: CC(C)(COCc1ccccc1)CC1(O)CCC2(CC1)OCCO2]

Into a 1 L 4-necked round-bottom flask purged and maintained with an inert atmosphere of nitrogen, was added tetrahydrofuran (33 ml) followed by tert-butyl lithium (2.6M in pentane, 25 ml, 42 mmol) at −90° C. The resulting mixture was stirred for 0.5 h at −90° C., then [(3-iodo-2,2-dimethylpropoxy)methyl]benzene (5 g, 16 mmol) in THF (15 ml) was added dropwise at −90° C. After stirring for 0.5 h, a solution of 1,4-dioxaspiro[4.5]decan-8-one (3.84 g, 24 mmol) in THF (15 ml) was added. The resulting solution was stirred at −90° C. for 1 h. The reaction was then quenched by the addition of NH₄Cl solution (sat. aq.) and then concentrated under vacuum. The residue was applied onto a silica gel column with ethyl acetate/hexane. This resulted in 5.0 g of the title compound as light yellow oil (90.0%). LCMS: m/z=335 [M+1]. ¹H-NMR (400 MHz, DMSO-d6) δ: 7.37-7.27 (m, 5H), 4.46 (s, 2H), 3.94 (s, 2H), 3.82 (s, 4H), 3.21 (s, 2H), 2.36 (t, J=7.2 Hz, 1H), 1.93 (t, J=7.2 Hz, 1H), 1.76-1.70 (m, 2H), 1.63-1.60 (m, 2H), 1.48-1.42 (m, 6H), 0.99 (s, 6H).

### Step-4: Synthesis of 3,3-dimethyl-1-oxaspiro[4.5]decan-8-one

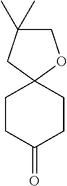

A stirred solution of 8-(3-(benzyloxy)-3-methylbutyl)-1,4-dioxaspiro[4.5]decan-8-ol (5.0 g, 14 mmol) in formic acid (25 ml) was heated to 80° C. for 2 h. The reaction mixture was cooled to rt and neutralized with aq. sodium bicarbonate solution and the product was extracted with ethyl acetate (3×50 ml), The combined organic layers were dried over Na₂SO₄ and concentrated under reduced pressure to obtain oily residue which was purified by column chromatography (60-120 silica gel, 0-20% ethyl acetate/hexane gradient). Fractions containing required compound were concentrated under reduced pressure to afford 1.2 g of the title compound (44.4%). ¹H-NMR (400 MHz, CDCl₃) δ: 3.60 (s, 2H), 2.75-2.67 (m, 2H), 2.26 (dd, J=3.6, 14.4 Hz, 2H), 2.16-2.10 (m, 2H), 1.89-1.81 (m, 2H), 1.68 (s, 2H), 1.16 (s, 6H).

### Step-5: Synthesis of 3,3-dimethyl-1-oxaspiro[4.5]dec-7-en-8-yl trifluoromethanesulfonate

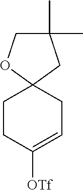 [SMILES: CC1(C)COC2(CC=C(OS(=O)(=O)C(F)(F)F)CC2)C1]

To a stirred solution of 3,3-dimethyl-1-oxaspiro[4.5]decan-8-one (5 g, 27 mmol) in DCM (40 ml) was added 2,6 di-tert-butyl-4-methyl pyridine (8.3 g, 40 mmol) at room temperature and the mixture was cooled to 0° C. Triflic anhydride (9.29 g, 32 mmol) was then added slowly at 0° C. The resulting solution was stirred at room temperature for 3-4 h. The reaction was concentrated under reduced pressure and was purified by column chromatography (60-120 silica gel, 0-20% ethyl acetate/hexane gradient). Fractions containing required compound were concentrated under reduced pressure to afford the title compound (50%). ¹H-NMR (400 MHz, CDCl₃) δ: 5.67-5.65 (m, 1H), 3.56 (dd, J=8.4, 13.2 Hz, 2H), 2.70-2.50 (m, 1H), 2.48-2.20 (m, 3H), 2.01-1.90 (m, 1H), 1.90-1.72 (m, 1H), 1.65 (d, J=6.4 Hz, 2H), 1.14 (d, J=4.0 Hz, 6H).

### Step-6: Synthesis of 2-(3,3-dimethyl-1-oxaspiro[4.5]dec-7-en-8-yl)-4,4,5,5-tetramethyl-1,3,2dioxaborolane

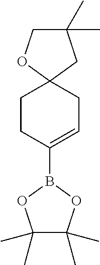

In a 35 ml sealed tube, purged and maintained with an inert atmosphere of argon, were placed 3,3-dimethyl-1-oxaspiro[4.5]dec-7-en-8-yl trifluoromethanesulfonate (4.33 g, 13 mmol), 1,4-dioxane (40 ml), 4,4,5,5-tetramethyl-2-(tetramethyl-1,3,2-dioxaborolan-2-yl)-1,3,2-dioxaborolane (3.57 g, 13 mmol) and potassium acetate (4.5 g, 41 mmol). The reaction mixture was degassed for 15 min under argon. Pd(dppf)Cl₂ (1.01 g, 1.3 mmol) was added and the resulting solution was stirred at 75° C. for 1 h. The reaction mixture was cooled to room temperature, diluted with water and extracted with 3×150 ml of ethyl acetate. The combined organic layers were dried over Na₂SO₄ and concentrated under reduced pressure to obtain 600 mg crude, which was purified by column chromatography (60-120 silica gel, 0-30% ethyl acetate/hexane gradient). Fractions containing required compound were concentrated under reduced pressure to the title compound (88%). ¹H-NMR (400 MHz, CDCl₃) δ: 6.48 (s, 1H), 3.54 (s, 2H), 2.36-2.25 (m, 4H), 1.70-1.50 (m, 4H), 1.25 (s, 12H), 1.10 (s, 6H).

### Step-7: Synthesis of 3-[3,3-dimethyl-1-oxaspiro[4.5]dec-7-en-8-yl]-1-(oxan-2-yl)-1H-pyrazole-4-carbaldehyde

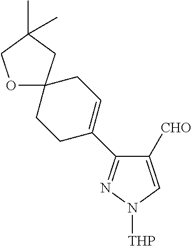

Into a 3-neck round-bottom flask, were placed 2-[3,3-dimethyl-1-oxaspiro[4.5]dec-7-en-8-yl]-4,4,5,5-tetramethyl-1,3,2-dioxaborolane (4.58 g, 15 mmol), 1,4-dioxane (40 ml), 3-iodo-1-(oxan-2-yl)-1H-pyrazole-4-carbaldehyde (4.0 g, 13 mmol), Cs₂CO₃ (12.7 g, 39 mmol), water (10 ml) the resulting mixture was degassed with argon for 20 min. Pd(dppf)Cl₂ (951 mg, 13 mmol) was then added and the resulting solution was stirred for 3 h at 75° C. The reaction mixture was concentrated under vacuum and extracted with 3×20 ml of ethyl acetate. The combined organic layers were washed with 3×20 ml of brine, dried over anhydrous sodium sulfate and concentrated under vacuum. The residue was applied onto a silica gel column with ethyl acetate/hexane as mobile phase. This resulted in 2.3 g of the title compound as yellow oil (51%). LCMS: m/z=345.0 [M+1].

### Step-8: Synthesis of 3-[3,3-dimethyl-1-oxaspiro[4.5]dec-7-en-8-yl]-1H-pyrazole-4-carbaldehyde

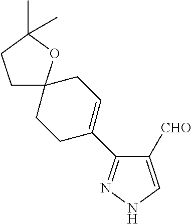 [SMILES: CC1(C)CCC2(CC=C(c3n[nH]cc3C=O)CC2)O1]

To a 100 ml round-bottom flask were placed 3-[3,3-dimethyl-1-oxaspiro[4.5]dec-7-en-8-yl]-1-(oxan-2-yl)-1H-pyrazole-4-carbaldehyde (1 g, 2.9 mmol), THF (4 ml), acetic acid (4 ml) and water (4 ml). The resulting solution was heated to 90° C. for 16 h. The reaction mixture was then concentrated under reduced pressure, then diluted with water and neutralized with sat. sodium bicarbonate solution. The mixture was extracted with 3×50 ml of ethyl acetate. The combined organic layers were dried over Na₂SO₄ and concentrated under reduced pressure to afford the title compound (77%). LCMS: m/z=261.31 [M+1].

### Step-9: Synthesis of N1-((3-(3,3-dimethyl-1-oxaspiro[4.5]dec-7-en-8-yl)-1H-pyrazol-4-yl)methyl)-N1,N2,N2-trimethylethane-1,2-diamine

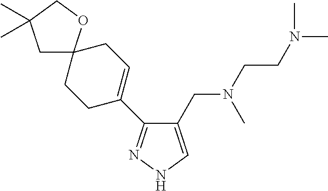

To a solution of 3-[3,3-dimethyl-1-oxaspiro[4.5]dec-7-en-8-yl]-1H-pyrazole-4-carbaldehyde (0.2 g, 0.76 mmol) in DCE (5 ml) was added N1,N1,N2-trimethylethane-1,2-diamine (0.14 ml, 1.1 mmol) followed by portion wise addition of NaBH(OAc)₃ (483 mg, 2.28 mmol). The reaction mixture was stirred at room temperature for 4 h. The reaction was then quenched by the addition of sodium bicarbonate solution (sat. aq.). The resulting mixture was washed with 3×5 ml of water and 3×5 ml of brine. The mixture was dried over anhydrous Na2SO4 and concentrated under vacuum. The residue was applied onto a silica gel column with dichloromethane/methanol as mobile phase to afford 0.2 g the title compound as light yellow oil (76.0%). LCMS: m/z=347.6 [M+1].

### Step-10: N1-((3-((5s,8s)-3,3-dimethyl-1-oxaspiro[4.5]decan-8-yl)-1H-pyrazol-4-yl)methyl)-N1,N2,N2-trimethylethane-1,2-diamine and N1-((3-((5r,8r)-3,3-dimethyl-1-oxaspiro[4.5]decan-8-yl)-1H-pyrazol-4-yl)methyl)-N1,N2,N2-trimethylethane-1,2-diamine

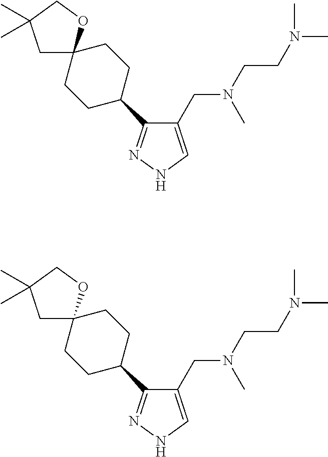

Into a 50 mL round-bottom flask, were placed N1-((3-(3,3-dimethyl-1-oxaspiro[4.5]dec-7-en-8-yl)-1H-pyrazol-4-yl)methyl)-N1,N2,N2-trimethylethane-1,2-diamine (0.6 g, 1.7 mmol), THF (25 ml), MeOH (2 ml) and Pd(OH)₂ (600 mg). H₂ gas was bubbled in to the reaction mixture for 2 h. After completion of reaction, the reaction mixture was filtered through Celite and then washed with methanol (2×25 ml). The combined filtrate and washing were concentrated under reduced pressure to afford the crude. The isomers were separated by prep-HPLC using X Bridge C18 column (250×19 mm) and 0.1% TFA in water, 100% acetonitrile mobile phase to afford two fractions: trans (150 mg_TFA salt), cis (380 mg_TFA salt). Trans: LCMS: m/z=349.6 [M+1]. ¹H-NMR (400 MHz, D2O) δ: 7.66 (s, 1H), 4.25 (s, 2H), 3.52 (s, 4H), 3.44 (s, 2H), 2.84 (s, 6H), 2.70 (s, 4H), 1.81-1.76 (t, 4H), 1.68 (s, 2H), 1.49-1.45 (t, 4H), 0.98 (s, 6H). Cis: LCMS: m/z=349.6 [M+1]. ¹H-NMR (400 MHz, D2O) δ: 7.77 (s, 1H), 4.30 (s, 2H), 3.55 (s, 4H), 3.43 (s, 2H), 3.22 (s, 3H), 2.86 (s, 6H), 2.59 (s, 4H), 1.93-1.86 (m, 2H), 1.65-1.40 (m, 8H), 0.97 (s, 6H).

### Example B-24. Synthesis of N1-((3-((5s,8s)-3,3-dimethyl-1-oxaspiro[4.5]decan-8-yl)-5-methyl-1H-pyrazol-4-yl)methyl)-N1-methylethane-1,2-diamine and N1-((3-((5r,8r)-3,3-dimethyl-1-oxaspiro[4.5]decan-8-yl)-5-methyl-1H-pyrazol-4-yl)methyl)-N1-methylethane-1,2-diamine (Compound 51 and 52)

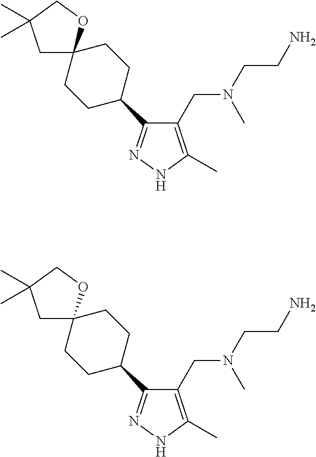 [SMILES: Cc1[nH]nc([C@H]2CC[C@@]3(CC2)CC(C)(C)CO3)c1CN(C)CCN ; Cc1[nH]nc([C@H]2CC[C@]3(CC2)CC(C)(C)CO3)c1CN(C)CCN]

### Step-1: Synthesis of 3-bromo-5-methyl-1H-pyrazole-4-carbaldehyde

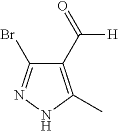

To the previously cooled (0-5° C.) solution of 5-methyl-2,4-dihydro-3H-pyrazol-3-one (1.5 g, 1.53 mmol) in dry DMF (3.7 g, 5.20 mmol), POBr₃ (14.9 g, 5.20 mmol) was added portion wise under nitrogen atmosphere. After complete addition of starting material, the reaction mixture was heated to 120° C. for 1 h. After complete consumption of starting material the reaction mixture was cooled to rt, water was added and the compound was extracted in ethyl acetate (2×50 ml). The combined organic layers were washed with sat. NaHCO₃ solution, then with brine solution, separated organic layer was dried over sodium sulphate, evaporate under vacuum to gives crude. The obtained crude material was purified by fractional column chromatography (0-20% EtOAc in hexane on silica gel mesh size (60-120 nm). Fractions containing pure product were combined, evaporated under vacuum to afford the title compound as an off-white solid (75%). LCMS: m/z=189 [M+1]. ¹H NMR (500 MHz, DMSO-d6) δ: 13.6 (s, 1H), 9.7 (s, J=1.1 Hz, 1H), 2.5 (s, J=1.8 Hz, 3H).

### Step-2: Synthesis of 3-bromo-5-methyl-1-(tetrahydro-2H-pyran-2-yl)-1H-pyrazole-4-carbaldehyde

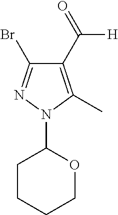

3-bromo-5-methyl-1H-pyrazole-4-carbaldehyde (3 g, 1.59 mmol) was taken up in dry THF (40 ml) and chloroform (120 ml) mixture, cooled it to 0-5° C. under nitrogen atmosphere. PTSA (6 g, 3.17 mmol) was added and the reaction mixture was stirred for 10-15 min. 3,4-dihydro-2H-pyran (4 g, 4.76 mmol) was added dropwise to the reaction mixture by maintaining the temperature to 0-5° C. Following addition, the ice bath was removed and the reaction mixture was stirred at rt for 4 h. After complete consumption of starting material, the reaction was quenched by the addition of sat. NaHCO₃ solution. The compound was extracted using EtOAc (3×50 ml). The combined organic layers were washed with brine solution, dried over sodium sulphate, and evaporated under vacuum. The crude material was then purified by fractional column chromatography (8% EtOAc in hexane on silica gel mesh size 60-120 to afford the title compound as a faint yellow viscous liquid (93%). LCMS: m/z=273 [M+1].

### Step-3: Synthesis of 3-(3,3-dimethyl-1-oxaspiro[4.5]dec-7-en-8-yl)-5-methyl-1H-pyrazole-4-carbaldehyde

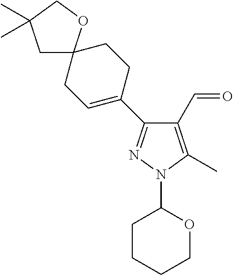 [SMILES: Cc1c(C=O)c(C2=CCC3(CC2)CC(C)(C)CO3)nn1C1CCCCO1]

Under continuous purging of argon gas 3-(3,3-dimethyl-1-oxaspiro[4.5]dec-7-en-8-yl)-5-methyl-1-(tetrahydro-2H-pyran-2-yl)-1H-pyrazole-4-carbaldehyde (4 g, 1.47 mmol), 2-(2,2-dimethyl-1-oxaspiro[4.5]dec-7-en-8-yl)-4,4,5,5-tetramethyl-1,3,2-dioxaborolane (4.1 g, 1.90 mmol) and aq. Na₂CO₃ (7.7 g, 7.325 mmol) were added to a 3-neck round bottom flask. Purging was continued for further 30 min. Pd(dppf)Cl₂ (1.07 g, 0.147 mmol) was added and the reaction mixture was heated to 80° C. for 16 h. After complete consumption of starting material, the reaction mixture was cooled to room temperature, water was added and the compound was extracted in ethyl acetate (2×50 ml). The combined organic layer were washed with brine solution, dried over sodium sulphate, and evaporated under vacuum. The crude material was then purified by column chromatography (0-20% EtOAc in hexane on silica gel mesh size 60-120) to afford the title compound as an off-white solid (76%). LCMS: m/z=358 [M+1].

### Step-4: Synthesis of 3-(3,3-dimethyl-1-oxaspiro[4.5]dec-7-en-8-yl)-5-methyl-1H-pyrazole-4-carbaldehyde

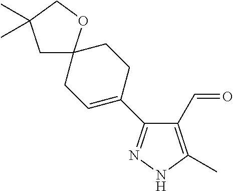 [SMILES: Cc1[nH]nc(C2=CCC3(CC2)CC(C)(C)CO3)c1C=O]

3-(3,3-dimethyl-1-oxaspiro[4.5]dec-7-en-8-yl)-5-methyl-1H-pyrazole-4-carbaldehyde (4 g, 18.59 mmol) was heated to 80° C. in an equivalent amount of acetic acid, water and THF (30 ml, 1:1:1) for 16 h. After complete consumption of starting material, the reaction mixture was cooled to rt and sat. NaHCO₃ solution was added to pH˜8. The compound was then extracted into EtOAc. The combined organic layers were washed with brine solution, dried over sodium sulphate, evaporated under vacuum. The crude material was then purified by column chromatography (0-30% EtOAc in hexane on silica gel mesh size 60-120 to afford the title compound as a faint yellow liquid (86%). LCMS: m/z=275.6 [M+1].

### Step-5: Synthesis of tert-butyl (2-(((3-(3,3-dimethyl-1-oxaspiro[4.5]dec-7-en-8-yl)-5-methyl-1H-pyrazol-4-yl)methyl)(methyl)amino)ethyl)carbamate

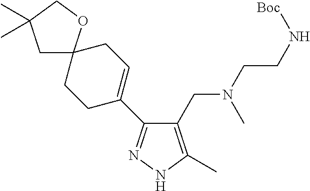 [SMILES: Cc1[nH]nc(C2=CCC3(CC2)CC(C)(C)CO3)c1CN(C)CCNC(=O)OC(C)(C)C]

Sodium triacetoxy borohydride (1.9 g, 9.11 mmol) was added portionwise to the mixture of 3-(3,3-dimethyl-1-oxaspiro[4.5]dec-7-en-8-yl)-5-methyl-1H-pyrazole-4-carbaldehyde (0.5 g, 1.82 mmol) and tert-butyl(2-(methylamino)ethyl)carbamate (0.58 g, 2.73 mmol) in a dry ethylene dichloride (10 ml) at 0-5° C. under nitrogen atmosphere. After complete addition of sodium triacetoxy borohydride reaction mixture was stirred at rt for 16 h. Progress of reaction was monitored on TLC (10% MeOH in DCM). After complete consumption of starting material, water was added and the pH of the aqueous layer was adjusted to ˜8 using sat. NaHCO₃ solution. The compound was then extracted in DCM (3×25 ml). The combined organic layers were then washed with brine solution, separated, dried over sodium sulphate, evaporate under vacuum. The crude material was then purified by column chromatography (0-40% EtOAc in hexane on neutral aluminato afford the title compound as a sticky liquid (54%). LCMS: m/z=433.16 [M+1].

### Step-6: tert-butyl (2-(((3-((5r,8r)-3,3-dimethyl-1-oxaspiro[4.5]decan-8-yl)-5-methyl-1H-pyrazol-4-yl)methyl)(methyl)amino)ethyl)carbamate and tert-butyl (2-(((3-((5s,8s)-3,3-dimethyl-1-oxaspiro[4.5]decan-8-yl)-5-methyl-1H-pyrazol-4-yl)methyl)(methyl)amino)ethyl)carbamate

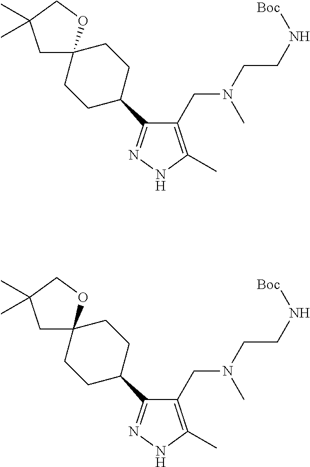 [SMILES: Cc1[nH]nc([C@H]2CC[C@]3(CC2)CC(C)(C)CO3)c1CN(C)CCNC(=O)OC(C)(C)C ; Cc1[nH]nc([C@H]2CC[C@@]3(CC2)CC(C)(C)CO3)c1CN(C)CCNC(=O)OC(C)(C)C]

Tert-butyl (2-(((3-(3,3-dimethyl-1-oxaspiro[4.5]dec-7-en-8-yl)-5-methyl-1H-pyrazol-4-yl)methyl)(methyl)amino)ethyl)carbamate (0.5 g, 11.97 mmol) was stirred in THF (10 ml) & MeOH (4 ml) by adding Pd(OH)₂ (250 mg) under continuous purging of hydrogen gas for 1 h at rt. The reaction mixture was filtered through Celite, washed with a mixture of methanol:DCM. The filtrate was evaporated under vacuum and the crude material was purified by fractional column chromatography (0-2% MeOH in DCM on neutral alumina) to afford the title compound with two regio isomers in the ratio of 75:15. The isomers were separated by prep-HPLC using YMC TRIATC18 column (150×20 mm), 5 and 0.02% NH₃ in water, 100% acetonitrile mobile phase to afford two fractions: trans (40 mg), cis (160 mg). LCMS: m/z=435.8 [M+1].

### Step-7: N1-((3-((5s,8s)-3,3-dimethyl-1-oxaspiro[4.5]decan-8-yl)-5-methyl-1H-pyrazol-4-yl)methyl)-N1-methylethane-1,2-diamine and N1-((3-((5r,8r)-3,3-dimethyl-1-oxaspiro[4.5]decan-8-yl)-5-methyl-1H-pyrazol-4-yl)methyl)-N1-methylethane-1,2-diamine

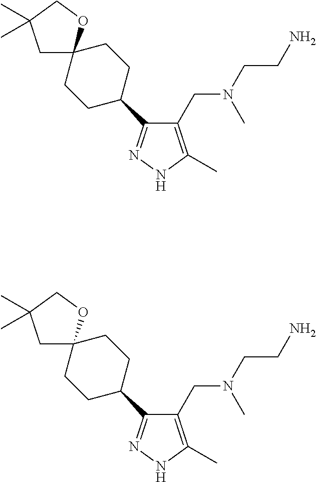 [SMILES: Cc1[nH]nc([C@H]2CC[C@@]3(CC2)CC(C)(C)CO3)c1CN(C)CCN ; Cc1[nH]nc([C@H]2CC[C@]3(CC2)CC(C)(C)CO3)c1CN(C)CCN]

Tert-butyl(2-(((3-((5s,8s)-3,3-dimethyl-1-oxaspiro[4.5]decan-8-yl)-5-methyl-1H-pyrazol-4-yl)methyl)(methyl) amino)ethyl)carbamate (160 mg, 0.369 mmol) in 4M HCl in isopropanol (4 ml) was stirred in a sealed tube for 24 h at rt. After complete consumption of starting material, solvent was evaporated under vacuumed the obtained solid was titrated in pentane and diethyl ether to give title compound 130 mg_HCl salt. LCMS: m/z=336.0 [M+1]. ¹H NMR (400 MHz, D₂O) δ: 4.25 (s, 2H), 3.35-3.45 (m, 6H), 2.70-2.74 (m, 4H), 2.23 (s, 3H), 1.84-1.87 (m, 2H), 1.43-1.51 (s, 8H), 0.95 (s, 6H).

Tert-butyl (2-(((3-((5r,8r)-3,3-dimethyl-1-oxaspiro[4.5]decan-8-yl)-5-methyl-1H-pyrazol-4-yl)methyl) (methyl) amino)ethyl)carbamate (40 mg, 0.092 mmol) in 4M HCl in isopropanol (2 ml) was stirred in a sealed tube for 24 h at rt. After complete consumption of starting material, solvent was evaporated under vacuum and the obtained solid was titrated in pentane and diethyl ether to give the title compound 28 mg HCl salt. LCMS: m/z=335.8 [M+1]. ¹H NMR (400 MHz, D₂O) δ: 4.22 (s, 2H), 3.47 (s, 2H), 3.36-3.45 (m, 4H), 2.74 (s, 3H), 2.58-2.63 (m, 1H), 2.38 (s, 3H), 1.84-1.73 (m, 4H), 1.68 (s, 2H), 1.39-1.52 (m, 4H), 0.99 (s, 6H).

### Example B-25. Synthesis of N1-((1-((5s,8s)-3,3-dimethyl-1-oxaspiro[4.5]decan-8-yl)-1H-pyrazol-5-yl)methyl)-N1,N2-dimethylethane-1,2-diamine and N1-((1-((5r,8r)-3,3-dimethyl-1-oxaspiro[4.5]decan-8-yl)-1H-pyrazol-5-yl)methyl)-N1,N2-dimethylethane-1,2-diamine (Compounds 49 and 50)

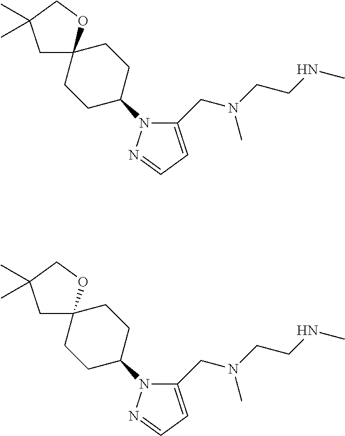

### Step-1: Synthesis of (3,3-dimethyl-1-oxaspiro[4.5]decan-8-yl)hydrazine

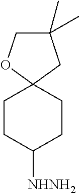

A stirred solution of 3,3-dimethyl-1-oxaspiro[4.5]decan-8-one (0.5 g, 2.7 mmol) in hexane (6 ml) was added tert-butyl hydrazine carboxylate (363 mg, 2.7 mmol) under nitrogen atmosphere. The reaction mixture was heated at 50° C. for 10 min and then allowed to cool to rt. The white precipitate that had formed was removed by filtration and was washed with cold hexane. The white solid was then treated with BH₃ (2.7 ml, 2.7 mmol) and the reaction mixture was stirred at rt for 20 min at which time the mixture was treated with 6N HCl (8 ml) and the reaction mixture was heated to 100° C. for 20 min. The resulting mixture was concentrated under vacuum and triturated with pentane to afford 400 mg of the title compound (73.0%). LCMS: m/z=199.3 [M+1]. ¹H-NMR (400 MHz, DMSO-d6) δ: 2.90-2.84 (m, 2H), 1.96 (m, 1H), 1.80-1.70 (m, 4H), 1.56-1.48 (m, 4H), 1.42-1.23 (m, 3H), 1.03 (s, 6H).

### Step-2: Synthesis of (E)-4-(dimethylamino)-1,1-dimethoxybut-3-en-2-one

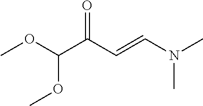

A stirred solution of 1,1-dimethoxy-N,N-dimethylmethanamine (5 g, 42 mmol) was mixed with 1,1-dimethoxypropan-2-one (4.95 g, 42 mmol) under nitrogen atmosphere. The reaction mixture was heated to 110° C. for 3 h. The methanol produced was removed by Dean-Stark apparatus. After completion the solution was cooled to rt and volatiles were removed under reduced pressure to obtain oily residue. The crude material was purified by column chromatography (60-120 silica gel, 0-5% Methanol/DCM) to afford 2.3 g of the title compound (68%). LCMS: m/z=174 [M+1]. ¹H-NMR (400 MHz, CDCl₃) δ: 7.61-7.58 (d, 1H), 5.19-5.16 (d, 1H), 4.43 (s, 1H), 3.26 (s, 6H), 3.10-3.08 (d, 3H), 2.79 (s, 3H), 1.15 (s, 6H).

### Step-3: Synthesis of 1-(3,3-dimethyl-1-oxaspiro[4.5]decan-8-yl)-1H-pyrazole-5-carbaldehyde

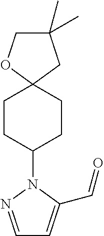 [SMILES: CC1(C)COC2(CCC(n3nccc3C=O)CC2)C1]

A stirred solution of (3,3-dimethyl-1-oxaspiro[4.5]decan-8-yl)hydrazine hydrochloride (2.0 g, 10 mmol) in ethanol (10 ml) was added (E)-4-(dimethylamino)-1,1-dimethoxybut-3-en-2-one (1.74 g, 10 mmol) under nitrogen atmosphere. The reaction mixture was heated at 85° C. for 12 h. The reaction solvent was evaporated and the resulting reaction mixture was dissolved in acetone (10 ml) and then treated with 6N HCl (10 ml). The resulting reaction mixture was stirred at room temperature for 2 h at which time it was cooled to room temperature and neutralized with aq. sodium bicarbonate solution. The product was extracted with ethyl acetate (3×10 ml), the combined organic layers were dried over Na₂SO₄ and concentrated under reduced pressure to obtain oily residue. The crude material was purified by column chromatography (60-120 silica gel, 0-50% ethyl acetate/hexane gradient) to afford the title compound (14%). ¹H-NMR (400 MHz, DMSO-d6) δ: 9.91 (s, 1H), 7.63 (d, 1H), 7.06 (d, 1H), 4.96-4.90 (m, 1H), 3.43 (d, 2H), 2.19-2.10 (m, 2H), 1.88-1.72 (m, 2H) 1.70-1.65 (m, 2H), 1.50-1.44 (m, 4H), 1.26 (s, 6H).

### Step-4: Compound 45 precursor: tert-butyl (2-(((1-((5s,8s)-3,3-dimethyl-1-oxaspiro[4.5]decan-8-yl)-1H-pyrazol-5-yl)methyl)(methyl)amino)ethyl)(methyl)carbamate and Compound 46 precursor: tert-butyl (2-(((1-((5r,8r)-3,3-dimethyl-1-oxaspiro[4.5]decan-8-yl)-1H-pyrazol-5-yl)methyl)(methyl)amino)ethyl)(methyl)carbamate

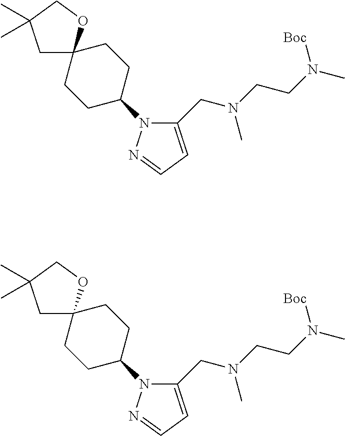

To a 50 ml round-bottom flask was added 1-(3,3-dimethyl-1-oxaspiro[4.5]decan-8-yl)-1H-pyrazole-5-carbaldehyde (0.380 g, 0.14 mmol, 1.00 equiv.), DCE (5 ml), tert-butyl N-methyl-N-[2-(methylamino)ethyl] carbamate (0.407 g, 0.21 mmol, 1.50 equiv.) and the reaction mixture was stirred for 15 min at which time NaBH(OAc)₃ (915 mg, 0.43 mmol, 3.01 equiv.) was added and the resulting solution was stirred at rt for 4 h. The reaction was then neutralized by the addition of sodium bicarbonate (sat. aq.). The resulting mixture was washed with 3×5 ml of water and 3×5 ml of brine. The mixture was dried over anhydrous Na2SO4 and concentrated under vacuum. The residue was purified by prep-HPLC (0.1% TFA in water) to afford two diastereomeric products. Cis (80 mg) LCMS: m/z=435.5 [M+1]. Trans precursor (50 mg) LCMS: m/z=435.5 [M+1].

### Step-5: N1-((1-((5s,8s)-3,3-dimethyl-1-oxaspiro[4.5]decan-8-yl)-1H-pyrazol-5-yl)methyl)-N1,N2-dimethylethane-1,2-diamine and N-((1-((5r,8r)-3,3-dimethyl-1-oxaspiro[4.5]decan-8-yl)-1H-pyrazol-5-yl)methyl)-N,N′-dimethylmethanediamine hydrochloride

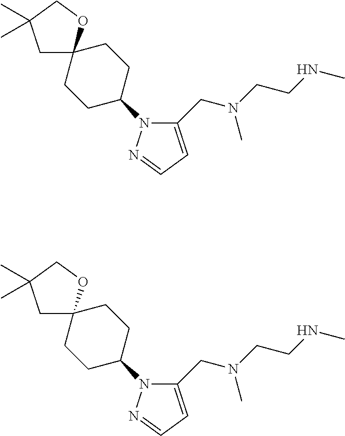

Tert-butyl((((1-(3,3-dimethyl-1-oxaspiro[4.5]decan-8-yl)-1H-pyrazol-5-yl)methyl)(methyl)amino) methyl) (methyl)carbamate (0.18 g, 0.41 mmol) and HCl in IPA (3 ml) was stirred at rt for 16 h and then concentrated under reduced pressure to obtain 85 mg compound which was triturated with n-pentane to afford the title compound as the hydrochloride salt (85.5%). LCMS: m/z=335.7 [M+1]. ¹H-NMR (400 MHz, D₂O) δ: 7.55-7.54 (d, J=1.6 Hz, 1H), 6.48-6.47 (d, J=2.0 Hz, 1H), 4.57-4.51 (d, 2H), 4.23-4.17 (m, 1H), 3.50-3.46 (m, 2H), 3.42-3.37 (m, 4H), 2.75 (s, 3H), 2.65 (s, 3H), 2.03-1.87 (m, 4H), 1.66-1.63 (m, 2H), 1.56-1.09 (m, 4H), 1.07 (s, 6H).

Tert-butyl((((1-(3,3-dimethyl-1-oxaspiro[4.5]decan-8-yl)-1H-pyrazol-5-yl)methyl)(methyl)amino)methyl) (methyl)carbamate (0.08 g, 0.18 mmol) and HCl in IPA (3 ml) was stirred at rt for 16 h and then concentrated under reduced pressure to obtain 33 mg compound which was triturated with n-pentane to afford the title compound as the hydrochloride salt (85.5%). LCMS: m/z=335.7 [M+1]. ¹H-NMR (400 MHz, D₂O) δ: 7.54 (d, J=1.6 Hz, 1H), 6.47 (d, J=2.0 Hz, 1H), 4.52 (s, 2H), 4.22-4.21 (m, 1H), 3.51-3.48 (m, 2H), 3.43-3.38 (m, 4H), 2.75 (s, 3H), 2.65 (s, 3H), 1.83-1.81 (m, 6H), 1.71 (s, 2H), 1.58-1.50 (m, 2H), 0.94 (s, 6H).

### Example B-26. Synthesis of N1-((3-(3,3-dimethyl-1-oxaspiro[4.5]dec-7-en-8-yl)-1H-pyrazol-4-yl)methyl)-N1,N2-dimethylethane-1,2-diamine (Compound 46)

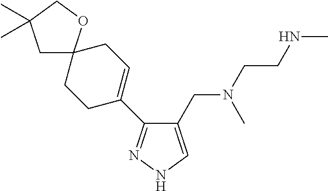

### Step-1: Synthesis of 3-[3,3-dimethyl-1-oxaspiro[4.5]dec-7-en-8-yl]-1-(oxan-2-yl)-1H-pyrazole-4-carbaldehyde

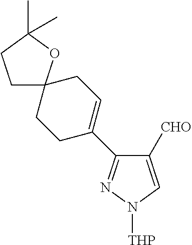

Into a 3-neck round-bottom flask, was placed 2-[3,3-dimethyl-1-oxaspiro[4.5]dec-7-en-8-yl]-4,4,5,5-tetramethyl-1,3,2-dioxaborolane (4.58 g, 15 mmol), 1,4-dioxane (40 ml), 3-iodo-1-(oxan-2-yl)-1H-pyrazole-4-carbaldehyde (4.0 g, 13 mmol), Cs₂CO₃ (12.7 g, 39 mmol), and water (10 ml). The resulting mixture was degassed with argon for 20 min followed by the addition of Pd(dppf)Cl₂ (951 mg, 13 mmol) and the resulting solution was stirred for 3 h at 75° C. The volatiles were concentrated under vacuum and extracted with 3×20 ml of ethyl acetate. The combined organic layer was washed with 3×20 ml of brine, dried over anhydrous Na₂SO₄ and concentrated under vacuum. The residue was applied onto a silica gel column with ethyl acetate/hexane as mobile phase. This resulted in 2.3 g of the title compound as yellow oil (51%). LCMS: m/z=345.0 [M+1].

### Step-2: Synthesis of 3-(2,2-dimethyl-1-oxaspiro[4.5]dec-7-en-8-yl)-1H-pyrazole-4-carbaldehyde

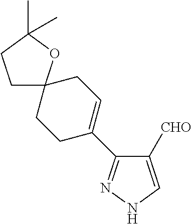 [SMILES: CC1(C)CCC2(CC=C(c3n[nH]cc3C=O)CC2)O1]

To a 100-ml round-bottom flask were placed 3-[3,3-dimethyl-1-oxaspiro[4.5]dec-7-en-8-yl]-1-(oxan-2-yl)-1H-pyrazole-4-carbaldehyde (1 g, 2.9 mmol), THF (4 ml), acetic acid (4 ml) and water (4 ml). The resulting solution was heated to 90° C. for 16 h. The reaction mixture was then concentrated under reduced pressure, diluted with water and neutralized with sat. sodium bicarbonate solution. The product was extracted with 3×50 ml of ethyl acetate, the combined organic layers were dried over Na₂SO₄ and concentrated under reduced pressure to afford the title compound (77%). LCMS: m/z=261.31 [M+1].

### Step-3: Synthesis of tert-butyl N-(2-[[(3-[3,3-dimethyl-1-oxaspiro[4.5]dec-7-en-8-yl]-1H-pyrazol-4-yl)methyl](methyl)amino]ethyl)-N-methylcarbamate

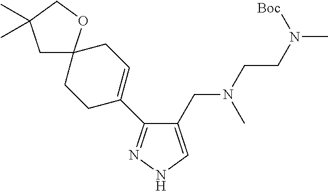

Into a 1000 ml round-bottom flask, was placed 3-[3,3-dimethyl-1-oxaspiro[4.5]dec-7-en-8-yl]-1H-pyrazole-4-carbaldehyde (0.2 g, 0.76 mmol, 1.00 equiv.), DCE (5 ml), and tert-butyl N-methyl-N-[2-(methylamino)ethyl]carbamate (216 mg, 0.11 mmol, 1.50 equiv.). NaBH(OAc)₃ (483 mg, 0.22 mmol, 3.01 equiv.) was added by batchwise and the resulting solution was stirred at rt for 4 h. The reaction was then quenched by the addition of sodium bicarbonate (sat. aq.). The resulting mixture was washed with 3×5 ml of water and 3×5 ml of brine. The mixture was dried over anhydrous Na2SO4 and concentrated under vacuum. The residue was applied onto a silica gel column with dichloromethane/methanol to afford 0.15 g of the title compound as light yellow oil (45%). LCMS: m/z=433.0 [M+1].

### Step-4: Synthesis of N1-((3-(3,3-dimethyl-1-oxaspiro[4.5]dec-7-en-8-yl)-1H-pyrazol-4-yl)methyl)-N1,N2-dimethylethane-1,2-diamine

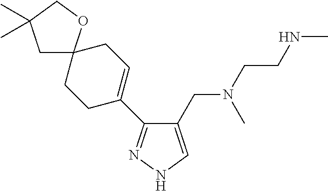

tert-butyl(2-(((3-(2,2-dimethyl-1-oxaspiro[4.5]decan-8-yl)-1H-pyrazol-4-yl)methyl)(methyl)amino)ethyl) (methyl)carbamate (0.15 g, 0.34 mmol) and HCl in IPA (3 ml) was stirred at rt for 16 h and then concentrated under reduced pressure to obtain crude 80 mg of the title compound as the HCl salt which was triturated with n-pentane to afford pure material (45.4%). LCMS: m/z=333.6 [M+1]. ¹H-NMR (400 MHz, D₂O) δ: 7.71 (s, 1H), 5.83 (s, 1H), 4.33-4.31 (m, 2H), 3.46 (s, 2H), 3.46 (s, 4H), 2.68 (s, 3H), 2.65 (s, 3H), 2.37-2.32 (m, 4H), 1.85-1.98 (m, 1H), 1.74-1.57 (m, 3H), 0.99 (s, 6H).

### Example B-27. Synthesis of N-((3-(4,4-bis(fluoromethyl)cyclohex-1-en-1-yl)-1H-pyrazol-4-yl)methyl)-N1,N2-dimethylethane-1,2-diamine (Compound 15)

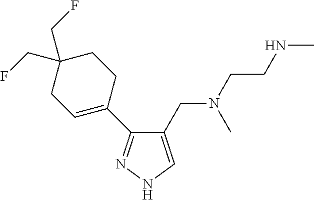 [SMILES: CNCCN(C)Cc1c[nH]nc1C1=CCC(CF)(CF)CC1]

### Step-1: Synthesis of (1,4-dioxaspiro[4.5]decane-8,8-diyl)bis(methylene)bis (trifluoromethanesulfonate)

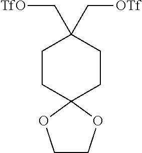 [SMILES: O=S(=O)(OCC1(COS(=O)(=O)C(F)(F)F)CCC2(CC1)OCCO2)C(F)(F)F]

Triflic anhydride (22.32 g, 79.1 mmol) was added dropwise to a solution of (1,4-dioxaspiro [4.5]decane-8,8-diyl)dimethanol (4.0 g, 19.7 mmol) and pyridine (20 ml) in DCM (20 ml) at 0° C. The reaction mixture was allowed to stir for 3 h at rt. The reaction mixture was diluted with water (35 ml) and the product was extracted with ethyl acetate (3×30 ml). The combined organic layers were concentrated under reduced pressure to afford the crude title compound which was carried forward to next step without further purification.

### Step-2: Synthesis of 8,8-bis(fluoromethyl)-1,4-dioxaspiro[4.5]decane

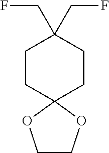 [SMILES: FCC1(CF)CCC2(CC1)OCCO2]

Tetrabutyl ammonium fluoride (17.04 g, 65.18 mmol) was added dropwise to a solution of (1,4-dioxaspiro[4.5]decane-8,8-diyl)bis(methylene)bis(trifluoromethanesulfonate) (7.6 g, 16.29 mmol) in dry THF (30 ml) at 0° C. The reaction mixture was stirred at rt for 3 h at which time water (50 ml) was added and the product was extracted with ethyl acetate (3×30 ml). The combined organic layers were dried over anhydrous sodium sulphate and concentrated under reduced pressure to afford a sticky mass which was crystallized in n-hexane to afford the title compound as a white solid (92%). ¹H NMR (400 MHz, DMSO-d6) δ: 4.41 (s, 2H), 4.29 (s, 2H), 3.86 (s, 4H), 1.59-1.56 (m, 4H), 1.50-1.49 (m, 4H).

### Step 3: Synthesis of 4,4-bis(fluoromethyl)cyclohexan-1-one

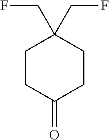 [SMILES: O=C1CCC(CF)(CF)CC1]

3M HCl (15 ml) was added dropwise to a solution of 8,8-bis(fluoromethyl)-1,4-dioxaspiro[4.5]decane (3.1 g, 15.03 mmol) in THF (15 ml) at rt. The reaction mixture was stirred for 16 h at rt. Solvents were evaporated and the crude material was diluted with ethyl acetate, washed with bicarbonate solution and the organic layer was separated and dried over sodium sulphate. The solvent was evaporated under reduced pressure and the crude material was purified by column chromatography using mobile phase 0-30% ethyl acetate in hexane to obtain the title compound (82%). ¹H NMR (400 MHz, DMSO-d6) δ: 4.54 (s, 2H), 4.42 (s, 2H), 2.32-2.30 (t, 4H), 1.72-1.68 (t, 4H).

### Step 4: Synthesis of 4,4-bis(fluoromethyl)cyclohex-1-en-1-yl trifluoromethanesulfonate

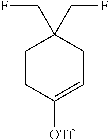 [SMILES: O=S(=O)(OC1=CCC(CF)(CF)CC1)C(F)(F)F]

A solution of 4,4-bis(fluoromethyl)cyclohexan-1-one (1.4 g, 8.63 mmol) in DCM (15 ml) was cooled to 0° C. and treated with 2,6-di-tert-butyl-4-methylpyridine (3.54 g, 17.26 mmol). The reaction was stirred for 15 min and triflic anhydride (6.08 g, 21.58 mmol) was added dropwise. The reaction mixture was allowed to stir at rt for 4 h. The solvent was evaporated under vacuum to obtain crude product which was purified by column chromatography using mobile phase 0-10% ethyl acetate in hexane (55%). ¹H NMR (400 MHz, DMSO-d6) δ: 5.90-5.88 (t, J=4.0 Hz, 1H), 4.40 (s, 2H), 4.28 (s, 2H), 2.39-2.35 (m, 2H), 2.12-2.11 (m, 2H), 1.74-1.71 (m, 2H).

### Step 5: Synthesis of 2-(4,4-bis(fluoromethyl)cyclohex-1-en-1-yl)-4,4,5,5-tetramethyl-1,3,2-dioxaborolane

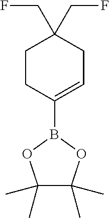 [SMILES: CC1(C)OB(C2=CCC(CF)(CF)CC2)OC1(C)C]

PdCl₂(dppf) (350 mg, 0.475 mmol) was added to a argon gas purged solution of 4,4-bis(fluoromethyl)cyclohex-1-en-1-yl trifluoromethanesulfonate (1.4 g, 4.75 mmol), potassium acetate (1.4 g, 14.27 mmol), bis-pinacolato diborane (1.2 g, 4.75 mmol) in dioxane (20 ml). The reaction mass was heated to 90° C. for 3 h at which time it was diluted with water and the product was extracted with ethyl acetate. The ethyl acetate was evaporated to obtain crude material which was purified by using mobile phase 0-15% ethyl acetate in hexane to get title compound (69%). ¹H NMR (400 MHz, DMSO-d6) δ: 6.37 (s, 1H), 4.33 (s, 2H), 4.21 (s, 2H), 2.05-2.03 (m, 2H), 1.95 (m, 2H), 1.47-1.45 (t, 2H), 1.24 (s, 12H).

### Step 6: Synthesis of tert-butyl (2-(((3-(4,4-bis(fluoromethyl)cyclohex-1-en-1-yl)-1-(tetrahydro-2H-pyran-2-yl)-1H-pyrazol-4-yl)methyl)(methyl)amino)ethyl) (methyl)carbamate

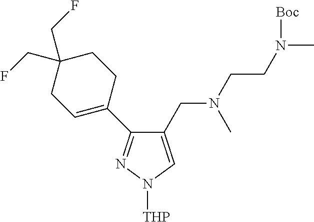

A solution of 2-(4,4-bis(fluoromethyl)cyclohex-1-en-1-yl)-4,4,5,5-tetramethyl-1,3,2-dioxaborolane (145 mg, 0.532 mmol), cesium carbonate (532 mg, 1.59 mmol), tert-butyl (2-(((3-iodo-1-(tetrahydro-2H-pyran-2-yl)-1H-pyrazol-4-yl)methyl)(methyl)amino)ethyl)(methyl)carbamate (254 mg, 0.532 mmol) in 1,4-dioxane (3 ml) and water (1 ml) was degassed using argon. PdCl₂(dppf) (39 mg, 0.053 mmol) was added and reaction was heated up to 90° C. for 3 h. The reaction mixture was diluted with water and the product was extracted with ethyl acetate. The organic layers were evaporated to dryness and the crude was purified by column chromatography using mobile phase 0-3% MeOH in DCM to afford the title compound (38%). LCMS: m/z=497.12 [M+1].

### Step 7: Synthesis of N-((3-(4,4-bis(fluoromethyl)cyclohex-1-en-1-yl)-1H-pyrazol-4-yl)methyl)-N1,N2-dimethylethane-1,2-diamine

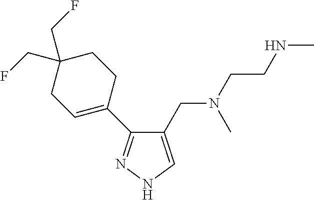 [SMILES: CNCCN(C)Cc1c[nH]nc1C1=CCC(CF)(CF)CC1]

HCl in IPA (3 ml) was added to a solution of tert-butyl (2-(((3-(4,4-bis(fluoromethyl)cyclohex-1-en-1-yl)-1-(tetrahydro-2H-pyran-2-yl)-1H-pyrazol-4-yl)methyl)(methyl)amino)ethyl)methyl)carbamate (0.090 g, 0.0258 mmol) in MeOH (2 ml) and allowed to stir at rt for 16 h. The solvent was evaporated under reduced pressure to obtained crude product, which was purified by prep-HPLC using X Bridge C18 column (250×19 mm) and 0.1% TFA in water, 100% acetonitrile mobile phase to afford the title compound as the TFA salt (58 mg). ¹H NMR (400 MHz, D₂O) δ: 7.83 (s, 1H), 6.01 (s, 1H), 4.48-4.44 (q, 2H), 4.397 (s, 2H), 3.36-4.32 (m, 2H), 3.50-3.45 (m, 4H), 2.83 (s, 3H), 2.79 (s, 3H), 2.49 (bs, 2H), 2.21 (bs, 2H), 2.81-2.78 (t, 2H).

### Example B-28. Synthesis of (5s,8s)-3,3-diethyl-8-(4-((methyl(2-(methylamino)ethyl)amino)methyl)-1H-pyrazol-3-yl)-1-oxaspiro[4.5]decan-2-one and (5r,8r)-3,3-diethyl-8-(4-((methyl(2-(methylamino)ethyl)amino)methyl)-1H-pyrazol-3-yl)-1-oxaspiro[4.5]decan-2-one (Compounds 33 and 34)

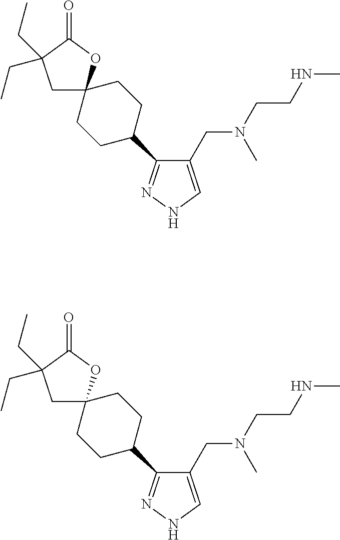

### Step-1: Synthesis of 1,7,10-trioxadispiro[2.2.46.23]dodecane

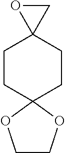 [SMILES: C1COC2(CCC3(CC2)CO3)O1]

To a stirred suspension of trimethyl sulfonium iodide (31.38 g, 153 mmol) in DMF (150 ml), sodium hydride (60% in mineral oil) (6.65 g, 277 mmol) was added under inert condition at 0° C. The reaction mixture was stirred for 30 min. at 0° C. followed by the addition of 1,4-dioxaspiro[4.5]decan-8-one (20 g, 128 mmol) in DMF (50 ml) dropwise. The reaction mixture was allowed to stirred at rt for 30 min at which time the reaction mixture was poured into ice-cold water slowly and extracted with ethyl acetate (3×100 ml), and the combined organic layers were collected and dried over sodium sulphate and concentrated under vacuum to dryness. The residue was purified by silica gel column chromatography using mobile phase (0%-10%) ethyl acetate in hexane to afford the title compound as pale light yellow liquid (92.6%). LCMS: m/z=171.1 [M+1]. ¹H NMR (400 MHz, CDCl₃) δ: 4.01-3.94 (m, 4H), 2.69 (s, 2H), 2.01-1.90 (m, 4H) 1.82-1.73 (m, 2H), 1.66-1.53 (m, 2H).

### Step-2: Synthesis of tert-butyl 3-(8-hydroxy-1,4-dioxaspiro[4.5]decan-8-yl)propanoate

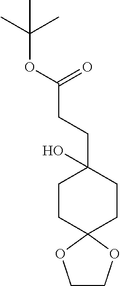 [SMILES: CC(C)(C)OC(=O)CCC1(O)CCC2(CC1)OCCO2]

A solution of diisopropylamine (11.60 g, 114.6 mmol) in dry THF (130 ml) was cooled to −78° C. followed by dropwise addition of n-butyl lithium (1.6M in hexane) under nitrogen atmosphere. The reaction mixture was brought to 0° C. for 30 min and again cooled to −78° C., then t-butyl acetate was added dropwise and stirred for a further 30 min at −78° C. Diethyl aluminum chloride (1M in hexane, 105.8 ml, 105.8 mmol) was then added and the resulting mixture was stirred for 30 min at −78° C. A solution of 1,7,10-trioxadispiro [2.2.46.23]dodecane (15 g, 88.2 mmol) in THF (90 ml) was added slowly added to reaction mass at −78° C. and the reaction mixture was stirred at −60 to −50° C. for 6 h. The reaction was quenched with saturated NH₄Cl solution and extracted with ethyl acetate (3×30 ml). The organic layer was collected and dried over sodium sulphate and concentrated in vacuum to dryness. The residue was purified by silica gel column chromatography using mobile phase (0%-40%) ethyl acetate in hexane to obtain the title compound as a pale yellow liquid (56%). LCMS: m/z=287.1 [M+1].

### Step-3: Synthesis of 1,4,9-trioxadispiro[4.2.48.25]tetradecan-10-one

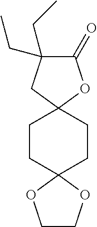 [SMILES: CCC1(CC)CC2(CCC3(CC2)OCCO3)OC1=O]

To a solution of 1,7,10-trioxadispiro[2.2.46.23]dodecane (2.8 g, 9.7 mmol) in chloroform (50 ml) was added p-toluene sulphonic acid (0.074 g, 0.38 mmol) at rt. The reaction mixture was heated up to 70° C. for 2 h. The reaction was poured into water (25 ml), neutralized with sodium bicarbonate solution and extracted with ethyl acetate (3×25 ml), the organic layer was dried over sodium sulphate and concentrated in vacuum to dryness. The residue was purified by silica gel column chromatography using mobile phase (0%-30%) ethyl acetate in hexane to obtain the title compound (81%). ESI-MS: m/z=264.0 [M+1]. ¹H NMR (400 MHz, CDCl₃) δ: 4.01-3.94 (m, 4H), 2.65-2.61 (t, 2H), 2.09-2.05 (t, 2H), 1.99-1.92 (m, 4H), 1.88-1.80 (m, 2H), 1.70-1.67 (m, 2H).

### Step-4: Synthesis of 11,11-diethyl-1,4,9-trioxadispiro[4.2.48.25]tetradecan-10-one

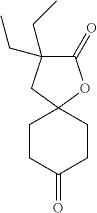 [SMILES: CCC1(CC)CC2(CCC(=O)CC2)OC1=O]

A solution of diisopropylamine (16.31 g, 160.8 mmol) in THF (160 ml) was cooled to −78° C. followed by dropwise addition of n-butyl lithium (1.6M in hexane, 100.5 ml, 160.8 mmol). The reaction mixture was brought to 0° C. for 30 min and again cool to −78° C. followed by addition of 1,4,9-trioxadispiro [4.2.48.25] tetradecan-10-one (5.7 g, 26.8 mmol) in THF (40 ml). The reaction mixture was stirred for additional 30 min at −78° C. and ethyl iodide (25.14 g, 160.8 mmol) and the reaction mixture was allowed to warm to rt and stir for 4 h. The reaction was quenched with sat. NH₄Cl solution and extracted with ethyl acetate (3×100 ml), the organic layer was collected and dried over sodium sulphate and concentrated in vacuum to dryness. The residue was purified by silica gel column chromatography using mobile phase (0%-30%) ethyl acetate in hexane to obtain the title compound (20.8%). ESI-MS: m/z=269.3 [M+1].

### Step 5: Synthesis of 3,3-diethyl-1-oxaspiro[4.5]decane-2,8-dione

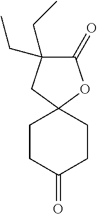 [SMILES: CCC1(CC)CC2(CCC(=O)CC2)OC1=O]

To a solution of 11,11-diethyl-1,4,9-trioxadispiro[4.2.48.25]tetradecan-10-one (1.4 g, 5.2 mmol) in THF (12 ml) was added 3N HCl solution (8 ml) at 0° C. The reaction mixture was stirred for 24 h and then diluted with water, neutralized with aq. sodium bicarbonate solution and extracted with ethyl acetate. The organic layer was dried and evaporated to obtain the crude which was purified by silica gel column chromatography using mobile phase (0%-30%) ethyl acetate in hexane to obtain the title compound (88.8%). ESI-MS: m/z=225.3 [M+1].

### Step-6: Synthesis of 3,3-diethyl-2-oxo-1-oxaspiro[4.5]dec-7-en-8-yl trifluoromethanesulfonate

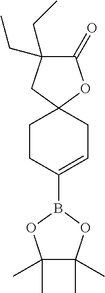 [SMILES: CCC1(CC)CC2(CC=C(B3OC(C)(C)C(C)(C)O3)CC2)OC1=O]

To a stirred solution of 3,3-diethyl-1-oxaspiro[4.5]decane-2,8-dione (0.480 g, 2.1 mmol) in tetrahydrofuran (6 ml) was added in LiHMDS (1M in THF, 2.57 ml) −78° C. and stirred for 45 min. 1,1,1-trifluoro-N-phenyl-N-(trifluoromethane)sulfonylmethane sulfonamide (0.765 g, 2.1 mmol) was added into the reaction mixture and stirred at rt for 1 h. The reaction was then quenched by the addition of 15 ml of sat. NH₄Cl (aq.). The resulting solution was extracted with 3×20 ml of ethyl acetate and the organic layer was dried and evaporated. The resulting crude was dissolved in ethylene glycol (20 ml) and extracted with hexane and the organic layer was concentrated under vacuum to get desired compound as light yellow oil that was used in the next step without further purification. ESI-MS: m/z=357.2 [M+1].

### Step-7: Synthesis of 3,3-diethyl-8-(4,4,5,5-tetramethyl-1,3,2-dioxaborolan-2-yl)-1-oxaspiro[4.5]dec-7-en-2-one

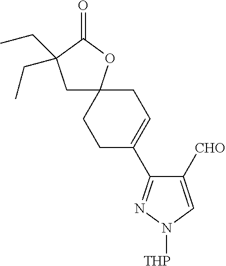

To a stirred solution of 3,3-diethyl-2-oxo-1-oxaspiro[4.5]dec-7-en-8-yl trifluoromethanesulfonate (0.700 g, 1.9 mmol), 4,4,5,5-tetramethyl-2-(tetramethyl-1,3,2-dioxaborolan-2-yl)-1,3,2-dioxaborolane (0.499 g, 1.9 mmol), potassium acetate (0.578 g, 5.8 mmol) in 1,4-dioxane (10 ml) was added Pd(dppf)Cl₂ (0.143 g, 0.19 mmol) under inert atmosphere. The resulting solution was stirred at 80° C. for 1 h. The reaction mixture diluted with water (25 ml), the resulting solution was extracted with (3×25 ml) of ethyl acetate and the organic layers combined. The resulting mixture was washed with (3×20 ml) of brine solution. The mixture was dried over anhydrous sodium sulfate. The residue was purified by silica gel column chromatography using mobile phase (0%-12%) ethyl acetate in hexane to obtain the title compound as yellow oil (56.3%). ESI-MS: m/z=335.43 [M+1].

### Step-8: Synthesis of 3-(3,3-diethyl-2-oxo-1-oxaspiro[4.5]dec-7-en-8-yl)-1-(tetrahydro-2H-pyran-2-yl)-1H-pyrazole-4-carbaldehyde

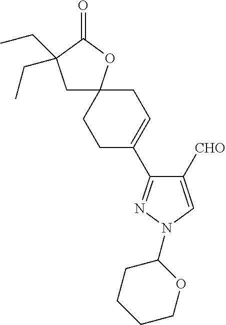 [SMILES: CCC1(CC)CC2(CC=C(c3nn(C4CCCCO4)cc3C=O)CC2)OC1=O]

To a stirred solution of 3,3-diethyl-8-(4,4,5,5-tetramethyl-1,3,2-dioxaborolan-2-yl)-1-oxaspiro[4.5]dec-7-en-2-one (1.48 g, 4.42 mmol) in 1,4-dioxane (17 ml) was added 3-iodo-1-(tetrahydro-2H-pyran-2-yl)-1H-pyrazole-4-carbaldehyde (1.219 g, 3.98 mmol), K₃PO₄ (2.81 g, 13.2 mmol) and water (2 ml) followed by purging with argon gas for 30 min. Pd(dppf)Cl₂ (0.323 g, 0.448 mmol) was added and the resulting solution was stirred for 2 h at 80° C. The reaction mixture was diluted with water (25 ml) and extracted with (3×50 ml) of ethyl acetate and the organic layers combined. The organic layer was washed with (3×50 ml) of brine solution, dried over anhydrous sodium sulfate and concentrated under vacuum. The residue was purified by silica gel column chromatography using mobile phase (0%-30%) ethyl acetate in hexane to afford the title compound (60.8%). LCMS: m/z=303.28 [M+1].

### Step-9: Synthesis of 3-(3,3-diethyl-2-oxo-1-oxaspiro[4.5]dec-7-en-8-yl)-1H-pyrazole-4-carbaldehyde

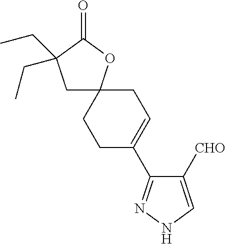 [SMILES: CCC1(CC)CC2(CC=C(c3n[nH]cc3C=O)CC2)OC1=O]

To as solution of 3-(3,3-diethyl-2-oxo-1-oxaspiro[4.5]dec-7-en-8-yl)-1-(tetrahydro-2H-pyran-2-yl)-1H-pyrazole-4-carbaldehyde (1.04 g, 2.69 mmol) in methanol (4 ml) was added concentrated HCl (4 ml) at 0° C. The reaction mixture was stirred at room temperature for 4 h at which time the reaction was neutralized with sodium bicarbonate solution, extracted with dichloromethane (3×25 ml), and the combined organic layers were dried over sodium sulphate and evaporate under vacuum to get desired crude product that was used in the next step without further purification.

### Step 10: Synthesis of tert-butyl (2-(((3-(3,3-diethyl-2-oxo-1-oxaspiro[4.5]dec-7-en-8-yl)-1H-pyrazol-4-yl)methyl)(methyl)amino)ethyl)(methyl)carbamate

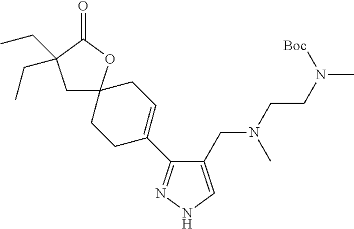 [SMILES: CCC1(CC)CC2(CC=C(c3n[nH]cc3CN(C)CCN(C)C(=O)OC(C)(C)C)CC2)OC1=O]

To a stirred solution of 3-(3,3-diethyl-2-oxo-1-oxaspiro[4.5]dec-7-en-8-yl)-1H-pyrazole-4-carbaldehyde (0.4 g, 1.32 mmol) and tert-butyl methyl(2-(methylamino)ethyl)carbamate (0.299 g, 1.58 mmol) in dry methanol (4 ml) was added with ZnCl₂ (0.009 g, 0.065 mmol) The reaction mixture was stirred at rt for 10 min followed by addition of triethylamine (0.668 g, 6.6 mmol). The reaction mixture was heated to 50° C. for 5 h, sodium cyanoborohydride (0.416 g, 6.7 mmol) was added at 0° C. The reaction mixture was stirred for 16 h at rt. The reaction was diluted with water and product was extracted in ethyl acetate (3×25 ml), the organic layer was collected and dried over sodium sulphate, concentrated in vacuum to dryness. The residue was purified by silica gel column chromatography using mobile phase (0%-4%) MeOH in DCM to give the title compound (45%). LCMS: m/z=475.61 [M+1].

### Step-11: Synthesis of tert-butyl (2-(((3-((5s,8s)-3,3-diethyl-2-oxo-1-oxaspiro[4.5]decan-8-yl)-1H-pyrazol-4-yl)methyl)(methyl)amino)ethyl)(methyl)carbamate and tert-butyl (2-(((3-((5r,8r)-3,3-diethyl-2-oxo-1-oxaspiro[4.5]decan-8-yl)-1H-pyrazol-4-yl)methyl)(methyl)amino)ethyl)(methyl)carbamate

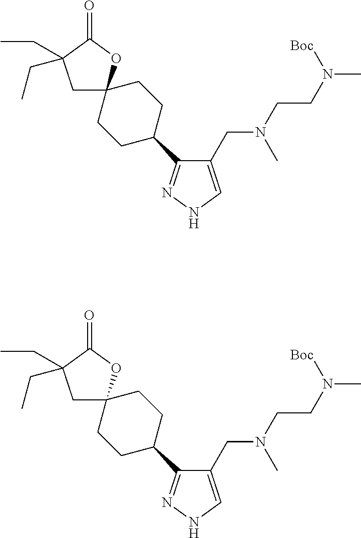

To a stirred suspension of 20% palladium hydroxide on charcoal (0.144 g) in THF (10 ml) under nitrogen atmosphere was added solution of tert-butyl (2-(((3-(3,3-diethyl-2-oxo-1-oxaspiro[4.5]dec-7-en-8-yl)-1H-pyrazol-4-yl)methyl)(methyl)amino)ethyl)(methyl)carbamate (0.284 g, 0.59 mmol) in THF (3 ml). The reaction mixture was purged with hydrogen gas for 2 h and filtered through Celite. The filtrate was concentrated in vacuum to dryness to obtain the title crude compound containing two regioisomers (0.290 g). The isomers were separated by prep-HPLC using X Bridge C18 column (250×19 mm) and 0.1% TFA in water, 100% acetonitrile mobile phase to afford two fractions: Fraction 1 (54 mg), Fraction 2 (120 mg). LCMS: m/z=477.7 [M+1].

### Step-12: Synthesis of (5s,8s)-3,3-diethyl-8-(4-((methyl(2-(methylamino)ethyl)amino)methyl)-1H-pyrazol-3-yl)-1-oxaspiro[4.5]decan-2-one

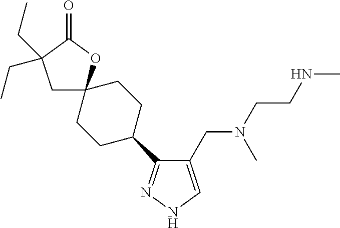 [SMILES: CCC1(CC)C[C@]2(CC[C@H](c3n[nH]cc3CN(C)CCNC)CC2)OC1=O]

tert-butyl (2-(((3-((5s,8s)-3,3-diethyl-2-oxo-1-oxaspiro[4.5]decan-8-yl)-1H-pyrazol-4-yl)methyl)(methyl)amino)ethyl)(methyl)carbamate (0.120 g, 0.26 mmol) and HCl in IPA was stirred for 16 h at rt. The reaction mixture was evaporated under vacuum followed by trituration with diethyl ether to get solid compound which was converted to free base by using polymer supported ammonium carbonate in methanol to get title compound (100%). LCMS: m/z=377.49 [M+1]. ¹H NMR (400 MHz, D₂O) δ: 7.59 (s, 1H), 4.15 (s, 2H), 3.36-3.22 (m, 4H), 2.68 (m, 1H), 2.60 (s, 3H), 2.05 (s, 3H), 1.85-1.80 (m, 4H), 1.56-1.48 (m, 6H), 1.41-1.33 (m, 4H), 0.66-0.63 (t, 6H).

### Step-13: Synthesis of (5r,8r)-3,3-diethyl-8-(4-((methyl(2-(methylamino)ethyl)amino)methyl)-1H-pyrazol-3-yl)-1-oxaspiro[4.5]decan-2-one

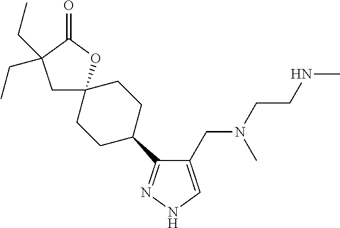 [SMILES: CCC1(CC)C[C@]2(CC[C@@H](c3n[nH]cc3CN(C)CCNC)CC2)OC1=O]

Tert-butyl (2-(((3-((5r,8r)-3,3-diethyl-2-oxo-1-oxaspiro[4.5]decan-8-yl)-1H-pyrazol-4-yl)methyl)(methyl)amino)ethyl)(methyl)carbamate (54 mg, 0.11 mmol) and HCl in IPA was stirred for 16 h at rt. The reaction mixture was evaporated under vacuum followed by trituration with diethyl ether to get solid compound which was converted to free base by using polymer supported ammonium carbonate in methanol to get the title compound. LCMS: m/z=377.49 [M+1]. ¹H NMR (400 MHz, D₂O) δ: 7.60 (s, 1H), 4.16 (s, 2H), 3.39-3.26 (m, 4H), 2.61 (s, 4H), 2.55 (s, 3H), 1.99 (s, 2H), 1.78-1.61 (m, 6H), 1.56-1.39 (m, 6H), 0.68-0.65 (t, 6H).

### Example B-29. Synthesis of N1-((3-(4,4-bis(fluoromethyl)cyclohexyl)-1H-pyrazol-4-yl)methyl)-N1,N2-dimethylethane-1,2-diamine (Compound 40)

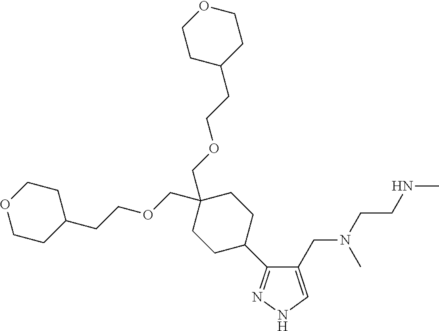 [SMILES: CNCCN(C)Cc1c[nH]nc1C1CCC(COCCC2CCOCC2)(COCCC2CCOCC2)CC1]

### Step-1: Synthesis of ethyl 2-(tetrahydro-4H-pyran-4-ylidene)acetate

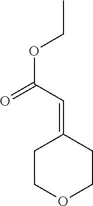

To a solution of tetrahydro-4H-pyran-4-one (0.5 g, 4.99 mmol) in DMF (5 ml) was added potassium carbonate (2.06 g, 14.9 mmol) at 0° C. The reaction mixture was stirred for 30 mins and then diethyl phosphoro ethyl acetate (1.119 g, 4.99 mmol) was added to the reaction mixture dropwise followed by heating at 80° C. for 2.5 h. The reaction was quenched with water (10 ml) and the product was extracted in ethyl acetate (3×10 ml), the combined organic layer was dried over anhydrous sodium sulphate and concentrated under reduced pressure to afford title compound that was used in the next reaction without further purification (72.9%). ¹H NMR (400 MHz, CDCl₃) δ: 5.70 (s, 1H), 4.20-4.15 (q, J=7.2 Hz, 2H), 3.81-3.748 (m, 4H), 3.05-3.02 (m, 2H), 2.37-2.34 (m, 2H), 1.30 (t, J=7.2 Hz, 3H).

### Step-2: Synthesis of ethyl 2-(tetrahydro-2H-pyran-4-yl)acetate

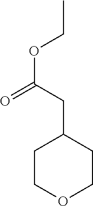

To a solution of ethyl 2-(tetrahydro-4H-pyran-4-ylidene)acetate (0.4 g, 2.33 mmol) in methanol (5 ml) was added NaBH₄ (0.198 g, 5.29 mmol) portionwise at 0° C. After 30 min of stirring at 0° C. NiCl₂ (0.12 g, 0.505 mmol) was added slowly. After stirring for 45 min the reaction solvent was evaporated under reduced pressure and diluted with water (10 ml) and the product was extracted with ethyl acetate (3×10 ml). The combined organic layer was concentrated under reduced pressure to afford the title compound (84%). ¹H NMR (400 MHz, CDCl₃) δ: 4.18-4.13 (q, J=7.2 Hz, 2H), 3.99-3.95 (m, 2H), 3.46-3.40 (m, 2H), 2.27-2.25 (m, 2H), 2.10-1.98 (m, 1H), 1.64 (s, 2H), 1.41-1.23 (m, 5H).

### Step-3: Synthesis of 2-(tetrahydro-2H-pyran-4-yl)ethan-1-ol

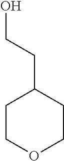

Lithium aluminum hydride (2M solution in THF, 40.66 ml, 81.3 mmol) was cooled at 0° C. and a solution of ethyl 2-(tetrahydro-2H-pyran-4-yl)acetate (14.0 g, 81.3 mmol) in THF (70 ml) was added dropwise. Ethyl acetate (20 ml) was added to the reaction mixture dropwise at 0° C. and the resulting mixture was allowed to stir for 16 h. The reaction mixture was filtered through Celite and the filtrate was concentrated to give crude compound. The crude material was purified by column chromatography using mobile phase 0-65% ethyl acetate in hexane to afford the title compound (66.1%). ¹H NMR (400 MHz, CDCl₃) δ: 5.71 (s, 1H), 4.18-4.15 (m, 2H), 3.81-3.75 (m, 4H), 3.05-3.02 (m, 2H), 2.37-2.34 (m, 2H), 1.32-1.31 (m, 3H).

### Step-4: Synthesis of 2-(tetrahydro-2H-pyran-4-yl)ethyl methanesulfonate

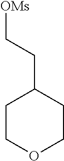 [SMILES: CS(=O)(=O)OCCC1CCOCC1]

Mesyl chloride (2.9 g, 25.3 mmol) was added dropwise to a solution of 2-(tetrahydro-2H-pyran-4-yl)ethan-1-ol (2.9 g, 22.3 mmol) in dichloromethane (20 ml) and triethylamine (2.76 g, 27.3 mmol) at 0° C. Then reaction mixture was stirred at rt for 4 h. A saturated solution of sodium bicarbonate (10 ml) was added to the reaction mixture and the product was extracted with dichloromethane (3×20 ml). The combined organic layer was dried over anhydrous sodium sulphate and concentrated under reduced pressure to afford the title compound which was carried forward to the next step without further purification.

### Step-5: Synthesis of 4-(2-bromoethyl)tetrahydro-2H-pyran

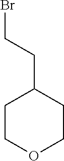

To a solution of 2-(tetrahydro-2H-pyran-4-yl)ethyl methanesulfonate (4.5 g, crude) in acetone (50 ml) was added lithium bromide (11.18 g, 129 mmol) and the resulting mixture was heated for 4 to 5 h at 50° C. The reaction solvent was evaporated under reduced pressure and the residue obtained was diluted with water and extracted with ethyl acetate. The combined organic layers were dried over anhydrous sodium sulphate and concentrated under reduced pressure to get crude product. Crude product was purified by column chromatography using mobile phase 0-65% ethyl acetate in hexane to afford the title compound. ¹H NMR (400 MHz, CDCl₃) δ: 4.01-3.97 (m, 2H), 3.49-3.39 (m, 4H), 1.86-1.76 (m, 3H), 1.65-1.62 (m, 2H), 1.37-1.36 (m, 2H).

### Step-6: Synthesis of 8,8-bis((2-(tetrahydro-2H-pyran-4-yl)ethoxy)methyl)-1,4-dioxaspiro[4.5]decane

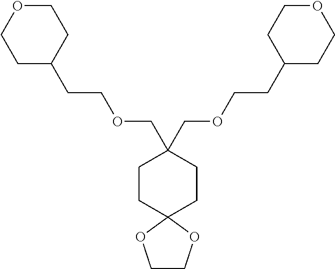 [SMILES: C1CC(CCOCC2(COCCC3CCOCC3)CCC3(CC2)OCCO3)CCO1]

Sodium hydride (60% in mineral oil, 475 mg, 19.76 mmol) was added portion wise to stirred solution of 1,4-dioxaspiro[4.5]decane-8,8-diyl)dimethanol (1.0 g, 4.94 mmol) in DMF (15 ml) at 0° C. and stirred for 30 min. 4-(2-bromoethyl)tetrahydro-2H-pyran (954 mg, 4.94 mmol) was added to the reaction and the reaction mixture was allowed to stir at rt for 3 h. The reaction was quenched by pouring over crushed ice and the product was extracted using ethyl acetate. The combined organic layers were dried over anhydrous sodium sulphate and concentrated under reduced pressure to get crude product. Crude product was purified by column chromatography using mobile phase 0-30% ethyl acetate in hexane to give title compound (22%). ¹H NMR (400 MHz, CDCl₃) δ: 3.98-3.94 (m, 8H), 3.46-3.36 (m, 8H), 3.28 (s, 4H), 1.68-1.50 (m, 14H), 1.33-1.30 (m, 8H).

### Step-7: Synthesis of 4,4-bis((2-(tetrahydro-2H-pyran-4-yl)ethoxy)methyl)cyclohexan-1-one

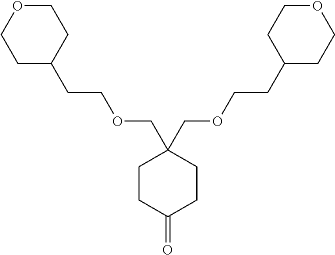 [SMILES: O=C1CCC(COCCC2CCOCC2)(COCCC2CCOCC2)CC1]

Ferric chloride hexahydrate (2.05 g, 7.6 mmol) was added to a solution of 8,8-bis((2-(tetrahydro-2H-pyran-4-yl)ethoxy)methyl)-1,4-dioxaspiro[4.5]decane (1.62 g, 3.8 mmol) in dichloromethane (20 ml) at 0° C. The reaction was allowed to stir at rt for 16 h. To the reaction mixture saturated solution of sodium bicarbonate (10 ml) was added and the product was extracted with dichloromethane (3×15 ml). The combined organic layers were passed through Celite and the filtrate was concentrated under reduced pressure to afford the crude compound. This was purified by column chromatography using mobile phase 0-50% ethyl acetate in hexane to give the title compound. (73%). ¹H NMR (400 MHz, CDCl₃) δ: 3.99-3.95 (m, 4H), 3.59-3.39 (m, 12H), 2.39-2.35 (m, 4H), 1.81-1.78 (m, 4H), 1.69-1.52 (m, 8H), 1.38-1.34 (m, 6H).

### Step-8: Synthesis of 4,4-bis((2-(tetrahydro-2H-pyran-4-yl)ethoxy)methyl)cyclohex-1-en-1-yl trifluoromethanesulfonate

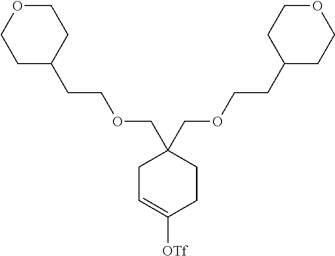 [SMILES: O=S(=O)(OC1=CCC(COCCC2CCOCC2)(COCCC2CCOCC2)CC1)C(F)(F)F]

LiHMDS (1M in solution in THF, 0.58 g, 3.46 mmol) was added dropwise to a solution of 4-bis((2-(tetrahydro-2H-pyran-4-yl)ethoxy)methyl)cyclohexan-1-one (1.01 g, 2.64 mmol) in dry THF (10 ml) at −78° C. and the resulting mixture was stirred for 30 min. N,N-Bis(trifluoromethylsulphonyl)aniline (0.94 g, 2.64 mmol) in THF (3 ml) was added and the reaction mixture allowed to stir for another 2 h. The reaction was quenched with saturated ammonium chloride solution (5 ml) and the product was extracted with ethyl acetate (3×10 ml). The combined organic layers were dried over sodium sulphate and concentrated under reduced pressure to afford crude product. The crude product was purified by column chromatography using mobile phase 18% ethyl acetate in hexane (35.3%). ¹H NMR (400 MHz, CDCl₃) δ: 5.70 (s, 1H), 3.99-3.95 (m, 4H), 3.47-3.39 (m, 8H), 3.36-3.23 (m, 4H), 2.32 (s, 2H), 2.10-2.07 (m, 2H), 1.75-1.50 (m, 10H), 1.37-1.33 (m, 6H).

### Step-9: Synthesis of 2-(4,4-bis((2-(tetrahydro-2H-pyran-4-yl)ethoxy)methyl)cyclohex-1-en-1-yl)-4,4,5,5-tetramethyl-1,3,2-dioxaborolane

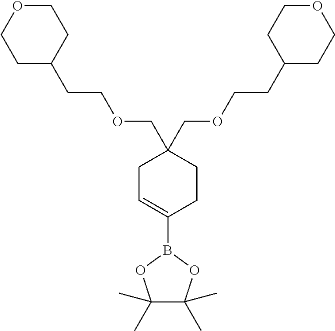 [SMILES: CC1(C)OB(C2=CCC(COCCC3CCOCC3)(COCCC3CCOCC3)CC2)OC1(C)C]

To a solution of 4,4-bis((2-(tetrahydro-2H-pyran-4-yl)ethoxy)methyl)cyclohex-1-en-1-yl trifluoromethanesulfonate (0.48 g, 0.933 mmol), potassium acetate (0.274 g, 2.79 mmol), 4,4,4′,4′,5,5,5′,5′-octamethyl-2,2′-bi(1,3,2-dioxaborolane) (0.26 g, 1.02 mmol) in 1,4-dioxane (5 ml) was added 1,1′-Pd(dppf)Cl₂ (0.068 g, 0.093 mmol). The resulting solution was stirred under argon degassing for 20 min and then heated at 80° C. for 2 h. After heating, the resulting brown mass was concentrated and the residue obtained was placed on silica gel for column chromatography using mobile phase 0-4% ethyl acetate in hexane to afford a pale yellow oil (64.2%). LCMS: m/z=493.7 [M+1].

### Step-10: Synthesis of tert-butyl (2-(((3-(4,4-bis((2-(tetrahydro-2H-pyran-4-yl)ethoxy)methyl)cyclohex-1-en-1-yl)-1-(tetrahydro-2H-pyran-2-yl)-1H-pyrazol-4-yl)methyl)(methyl)amino)ethyl)(methyl)carbamate

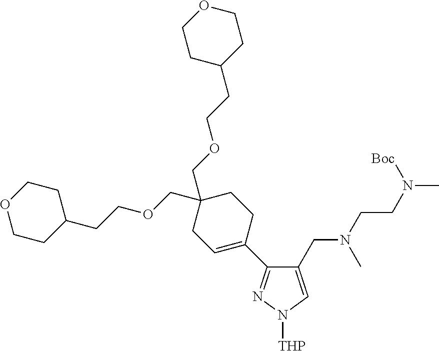

To a solution of 2-(4,4-bis((2-(tetrahydro-2H-pyran-4-yl)ethoxy)methyl)cyclohex-1-en-1-yl)-4,4,5,5-tetramethyl-1,3,2-dioxaborolane (0.295 g, 0.59 mmol), potassium phosphate (0.38 g, 1.8 mmol), tert-butyl (2-(((3-iodo-1-(tetrahydro-2H-pyran-2-yl)-1H-pyrazol-4-yl)methyl)(methyl)amino)ethyl)(methyl)carbamate (0.282 g, 0.598 mmol) in 1,4-dioxane (5 ml) and water (1.0 ml) was added Pd(dppf)Cl₂ (0.044 g, 0.582 mmol). The reaction mixture was heated at 75° C. for 2 h and the resulting brown mass was concentrated, water (15 ml) was added. The mixture was extracted with ethyl acetate (3×10 ml), dried over anhydrous sodium sulphate and concentrated under reduced pressure to give crude product. The crude product was purified by chromatography using stationary phase neutral alumina and mobile phase 0-2% MeOH in DCM to gives thick semi solid (58.2%). LCMS: m/z=718.3 [M+1]

### Step-11: tert-butyl(2-(((3-(4,4-bis((2-(tetrahydro-2H-pyran-4-yl)ethoxy)methyl)cyclohexyl)-1-(tetrahydro-2H-pyran-2-yl)-1H-pyrazol-4-yl)methyl)(methyl)amino)ethyl) (methyl)carbamate

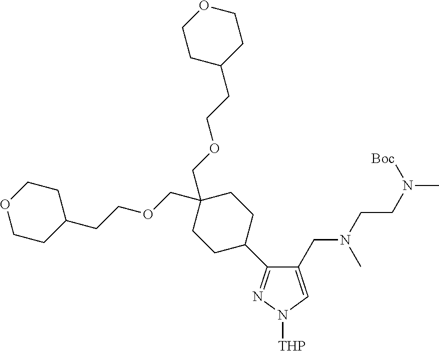

20% Palladium hydroxide on carbon (0.2 g) was added to a solution of tert-butyl (2-(((3-(4,4-bis((2-(tetrahydro-2H-pyran-4-yl)ethoxy)methyl)cyclohex-1-en-1-yl)-1-(tetrahydro-2H-pyran-2-yl)-1H-pyrazol-4-yl)methyl)(methyl)amino)ethyl)(methyl)carbamate (0.25 g, 0.348 mmol) in THF (5 ml). H₂ was bubbled though the reaction mixture until the starting was consumed at which time the reaction mixture was filtered through Celite. The filtrate was concentrated and the crude product was purified by chromatography using stationary phase neutral alumina and mobile phase 0-1.8% MeOH in DCM to give a thick semi-solid (47.9%). LCMS: m/z=720.50 [M+1].

### Step-12: Synthesis of N1-((3-(4,4-bis(fluoromethyl)cyclohexyl)-1H-pyrazol-4-yl)methyl)-N1,N2-dimethylethane-1,2-diamine

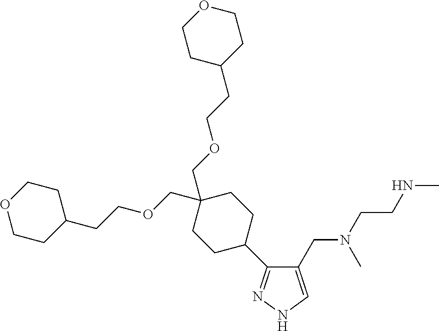 [SMILES: CNCCN(C)Cc1c[nH]nc1C1CCC(COCCC2CCOCC2)(COCCC2CCOCC2)CC1]

HCl in IPA (4 ml) was added to a solution of tert-butyl(2-(((3-(4,4-bis((2-(tetrahydro-2H-pyran-4-yl)ethoxy)methyl)cyclohexyl)-1-(tetrahydro-2H-pyran-2-yl)-1H-pyrazol-4-yl)methyl)(methyl)amino)ethyl) (methyl) carbamate (0.12 g, 0.016 mmol) in MeOH (3 ml). The solvent was evaporated under reduced pressure to obtained colorless oil which was triturated using n-pentane and diethyl ether to afford an off-white solid as the dihydrochloride salt. LCMS: m/z=534.77 [M+1]. ¹H NMR (400 MHz, D₂O) δ: 7.62 (s, 1H), 4.19 (s, 2H), 5.02-5.00 (m, 4H), 3.45-3.36 (m, 8H), 3.31-3.33 (m, 4H), 3.12 (s, 2H), 2.65 (s, 3H), 2.60 (m, 4H), 1.55-1.48 (m, 10H), 1.39-1.36 (m, 4H), 1.14-1.06 (m, 8H).

### Example B-30. Synthesis of N1-((3-(4-(methoxymethyl)-4-(((tetrahydro-2H-pyran-4-yl)oxy)methyl)cyclohexyl)-1H-pyrazol-4-yl)methyl)-N1,N2-dimethylethane-1,2-diamine (Compound 45)

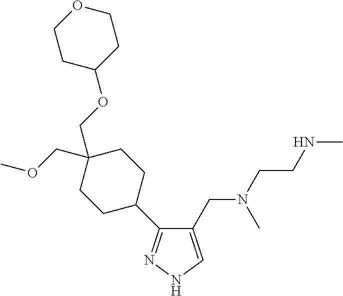

### Step-1: Synthesis of (8-(((tetrahydro-2H-pyran-4-yl)methoxy)methyl)-1,4-dioxaspiro[4.5]decan-8-yl)methanol

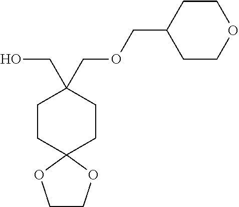 [SMILES: OCC1(COCC2CCOCC2)CCC2(CC1)OCCO2]

To a stirred solution of (1,4-dioxaspiro[4.5]decane-8,8-diyl)dimethanol (5 g, 24 mmol) in dry DMF (50 ml), sodium hydride (60%, 3.95 g, 160 mmol) was added portionwise at 0° C. under nitrogen atmosphere. The reaction mixture was stirred to 0-10° C. for 45 min, followed by the addition of 4-(bromomethyl) tetrahydro-2H-pyran (9.2 g, 51 mmol) dropwise in 1 h. The reaction mixture was allowed to stir at room temperature for 16 h. The reaction mixture was poured into ice-cold water and extracted with diethyl ether (3×50 ml). The organic extracts were combined, washed with brine (50 ml) and water (50 ml), dried over anhydrous Na₂SO₄, filtered and evaporated under reduced pressure to give a yellow viscous residue. The crude product was purified by silica gel column chromatography using mobile phase 0-70% ethyl acetate in hexane to afford the title compound as pale yellow viscous liquid (5.3%). ¹H NMR (400 MHz, CDCl₃) δ: 4.00 (m, 2H), 3.96 (s, 4H), 3.61-3.59 (m, 2H), 3.42-3.28 (m, 4H), 3.30-3.28 (d, J=8 Hz, 2H), 2.92-2.90 (m, 1H), 1.89-1.82 (m, 1H) 1.68-1.62 (m, 8H), 1.57-1.47 (m, 2H), 1.41-1.27 (m, 2H).

### Step-2: Synthesis of 8-(methoxymethyl)-8-(((tetrahydro-2H-pyran-4-yl)methoxy)methyl)-1,4-dioxaspiro[4.5]decane

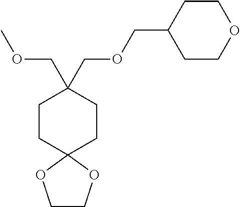 [SMILES: COCC1(COCC2CCOCC2)CCC2(CC1)OCCO2]

To a stirred solution of (8-(((tetrahydro-2H-pyran-4-yl)methoxy)methyl)-1,dioxaspiro[4.5]decan 8-yl)methanol (0.390 g, 1.3 mmol) in DMF (4 ml), sodium hydride (60% in mineral oil, 0.208 g, 8.0 mmol) was added portionwise at 0° C. under nitrogen atmosphere. The reaction mixture was stirred to 0-10° C. for 45 min followed by the addition iodomethane (0.369 g, 2.5 mmol) dropwise. The reaction mixture was allowed to stir at rt for 16 h at which time the reaction mixture was poured into ice-cold water and extracted with diethyl ether (3×20 ml). The organic extracts were combined, washed with brine (20 ml) and water (20 ml), dried over anhydrous Na₂SO₄, filtered and evaporated under reduced pressure to give a yellow viscous residue. The crude product was purified by silica gel column chromatography using mobile phase 0-30% ethyl acetate in hexane to afford the title compound as pale yellow viscous liquid (77.6%). ¹H NMR (400 MHz, CDCl₃) δ: 4.00-3.97 (m, 2H), 3.97-3.95 (m, 4H), 3.44-3.37 (m, 2H), 3.33 (s, 3H), 3.28-3.25 (m, 6H), 1.88-1.82 (m, 1H), 1.64-1.62 (m, 6H), 1.57-1.53 (m, 4H), 1.40-1.29 (m, 2H).

### Step-3: Synthesis of 4-(methoxymethyl)-4-(((tetrahydro-2H-pyran-4-yl)methoxy)methyl)cyclohexan-1-one

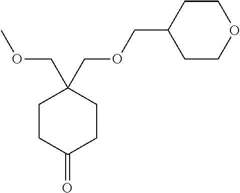 [SMILES: COCC1(COCC2CCOCC2)CCC(=O)CC1]

To a stirred solution of 8-(methoxymethyl)-8-(((tetrahydro-2H-pyran-4-yl)methoxy)methyl)-1,4-dioxaspiro[4.5]decane (0.550 g, 1.7 mmol) in DCM (5 ml) was added ferric chloride hexahydrate (0.880 g, 5.4 mmol) lot wise at 0-5° C. The mixture was stirred at room temperature for 4 h. The filtrate was neutralized with aq. sodium bicarbonate solution, the resulting suspension was filtered through Celite. The organic layer was separated and dried over anhydrous sodium sulphate and the solvent was evaporated to obtain a brown viscous liquid. The crude product was purified by silica gel column chromatography using mobile phase 0-40% ethyl acetate in hexane to afford the title compound as pale yellow viscous mass (67.6%). ¹H NMR (400 MHz, CDCl₃) δ: 4.01-3.95 (m, 2H), 3.44-3.41 (m, 2H), 3.38-3.33 (m, 7H), 3.30-3.28 (d, J=8.0 Hz, 2H), 2.38-2.34 (m, 4H), 1.89-1.83 (m, 1H), 1.81-1.77 (m, 4H), 1.41-1.29 (m, 3H).

### Step-4: Synthesis of 4-(methoxymethyl)-4-(((tetrahydro-2H-pyran-4-yl)methoxy)methyl)cyclohex-1-en-1-yl trifluoromethanesulfonate

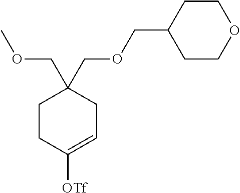 [SMILES: COCC1(COCC2CCOCC2)CC=C(OS(=O)(=O)C(F)(F)F)CC1]

To a stirred solution of 4-(methoxymethyl)-4-(((tetrahydro-2H-pyran-4-yl)methoxy) methyl) cyclohexan-1-one (0.319 g, 1.18 mmol) in dry THF (3 ml) cooled at −70° C. was added LiHMDS (1.53 ml, 1M solution in THF, 1.53 mmol) under nitrogen. Stirring was continued at −70° C. for 1 h. A solution of 1,1,1-trifluoro-N-phenyl-N-((trifluoromethyl)sulfonyl)methanesulfonamide (0.379 g, 1.06 mmol) in dried THF (1 ml) was added dropwise. The reaction mixture was allowed to warm to rt and stirred for 2 h. Saturated ammonium chloride solution (2 ml) was added and the mixture was concentrated to dryness. Water (10 ml) was poured into the residue and the mixture was extracted with ethyl acetate (4×20 ml). The organic extracts were combined, washed with water (20 ml), dried over anhydrous Na₂SO₄, filtered and evaporated under reduced pressure to give a pale brown viscous liquid. The crude product was purified by silica gel chromatography using a hexane:ethyl acetate (20%) to afford the title compound as pale yellow liquid (34%). ¹H NMR (400 MHz, CDCl₃) δ: 5.70-5.68 (m, 1H), 3.99-3.28 (m, 2H), 3.99-3.39 (m, 2H), 3.35-3.34 (m, 3H), 3.30-3.21 (m, 6H), 2.32-2.30 (m, 2H), 2.10-2.07 (m, 2H), 1.88-1.81 (m, 1H), 1.77-1.69 (m, 2H), 1.64-1.57 (m, 2H), 1.41-1.32 (m, 2H).

### Step-5: Synthesis of 2-(4-(methoxymethyl)-4-(((tetrahydro-2H-pyran-4-yl)methoxy)methyl)cyclohex-1-en-1-yl)-4,4,5,5-tetramethyl-1,3,2-dioxaborolane

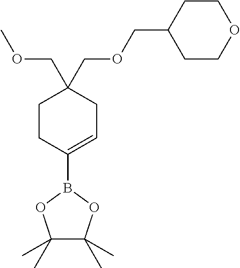 [SMILES: COCC1(COCC2CCOCC2)CC=C(B2OC(C)(C)C(C)(C)O2)CC1]

To a stirred solution of 4-(methoxymethyl)-4-(((tetrahydro-2H-pyran-4-yl)methoxy)methyl) cyclohex-1-en-1-yl trifluoromethanesulfonate (0.200 g, 0.49 mmol); 4,4,4′,4′,5,5,5′,5′-octamethyl-2,2′-bi(1,3,2-dioxaborolane) (0.126 g, 0.495 mmol) and potassium acetate (0.146 g, 1.48 mmol) in 1,4-dioxan (4 ml) purged and maintained with an inert atmosphere of argon was added Pd(dppf)Cl₂ (233 mg, 0.319 mmol) and heated at 80° C. for 30 min. Water (10 ml) was poured into the reaction mixture and extracted with ethyl acetate (3×10 ml). The organic extracts were combined, dried over anhydrous Na₂SO₄, filtered and evaporated under reduced pressure to give a brown viscous mass which was purified by silica gel column chromatography using mobile phase 0-50% ethyl acetate in hexane to afford the title compound (76.7%). LCMS: m/z=381.74 [M+1].

### Step-6: Synthesis of 3-(4-(methoxymethyl)-4-(((tetrahydro-2H-pyran-4-yl)methoxy)methyl)cyclohex-1-en-1-yl)-1-(tetrahydro-2H-pyran-2-yl)-1H-pyrazole-4-carbaldehyde

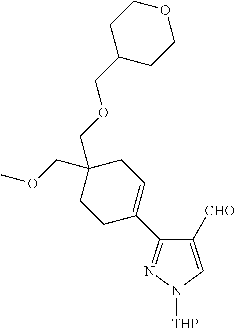

To a stirred solution of 2-(4-(methoxymethyl)-4-(((tetrahydro-2H-pyran-4-yl) methoxy) methyl) cyclohex-1-en-1-yl)-4,4,5,5-tetramethyl-1,3,2-dioxaborolane (0.140 g, 0.36 mmol), 3-iodo-1-(tetrahydro-2H-pyran-2-yl)-1H-pyrazole-4-carbaldehyde (0.101 g, 033 mmol) and potassium phosphate (0.234 g, 1.1 mmol) in 1,4-dioxan (1.8 ml) and water (0.2 ml) purged and maintained with an inert atmosphere of argon was added Pd(dppf)Cl₂ (0.026 g, 0.036 mmol) and heated at 80° C. for 2 h. Water (5 ml) was added into the reaction mixture and the product was extracted with ethyl acetate (3×10 ml). The organic extracts were combined, dried over anhydrous Na₂SO₄, filtered and evaporated under reduced pressure to give a brown viscous mass. The crude product was purified by silica gel column chromatography using mobile phase 0-50% ethyl acetate in hexane to afford the title compound as pale yellow viscous mass (44.0%). LCMS: m/z=433.7 [M+1].

### Step-7: Synthesis of 3-(4-(methoxymethyl)-4-(((tetrahydro-2H-pyran-4-yl)methoxy)methyl)cyclohex-1-en-1-yl)-1H-pyrazole-4-carbaldehyde

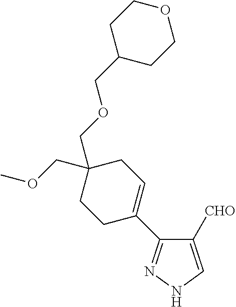 [SMILES: COCC1(COCC2CCOCC2)CC=C(c2n[nH]cc2C=O)CC1]

A solution of 3-(4-(methoxymethyl)-4-(((tetrahydro-2H-pyran-4-yl)methoxy)methyl)cyclohex-1-en-1-yl)-1-(tetrahydro-2H-pyran-2-yl)-1H-pyrazole-4-carbaldehyde (70 mg, 0.16 mmol) in dry THF (0.5 ml), water (0.5 ml), and acetic acid (0.5 ml) was heated to 80° C. for 16 h. The reaction mixture was diluted in ice-cold water (5 ml) and neutralized with aq. sodium bicarbonate solution and the compound was extracted with ethyl acetate (3×10 ml). The combined organic extracts were dried over sodium sulphate and concentrate under vacuum to get crude product which was purified by silica gel column chromatography using mobile phase (0-4%) MeOH in MDC to afford the title compound (48.2%). LCMS: m/z=349.6 [M+1].

### Step-8: Synthesis of tert-butyl (2-(((3-(4-(methoxymethyl)-4-(((tetrahydro-2H-pyran-4-yl) methoxy) methyl)cyclohex-1-en-1-yl)-1H-pyrazol-4-yl)methyl)(methyl) amino) ethyl)(methyl)carbamate

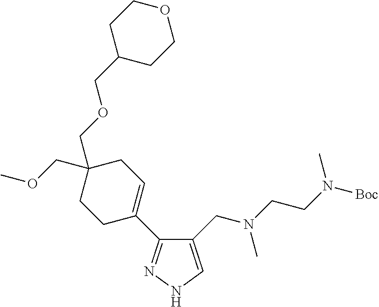 [SMILES: COCC1(COCC2CCOCC2)CC=C(c2n[nH]cc2CN(C)CCN(C)C(=O)OC(C)(C)C)CC1]

Sodium triacetoxyborohydride was added to a solution of 3-(4-(methoxymethyl)-4-(((tetrahydro-2H-pyran-4-yl) methoxy) methyl) cyclohex-1-en-1-yl)-1H-pyrazole-4-carbaldehyde (25 mg, 0.071 mmol) and tert-butyl methyl(2-(methylamino)ethyl) carbamate (20 mg, 1.07 mmol) in dry DCE (2 ml) at 0° C. The reaction mixture was stirred for 16 h at rt. The reaction mixture was diluted with cold water and the product was extracted with dichloromethane (3×10 ml). The combined organic extracts were dried over sodium sulphate and evaporated under vacuum to get crude product. The crude product was subjected to column chromatography using neutral alumina stationary phase and mobile phase as (0-2%) MeOH in DCM to afford the desired title compound (59.4%). ESI-MS: m/z=522[M+1].

### Step-9: Synthesis of tert-butyl (2-(((3-(4-(methoxymethyl)-4-(((tetrahydro-2H-pyran-4-yl) methoxy) methyl)cyclohexyl)-1H-pyrazol-4-yl)methyl)(methyl)amino)ethyl) (methyl)carbamate

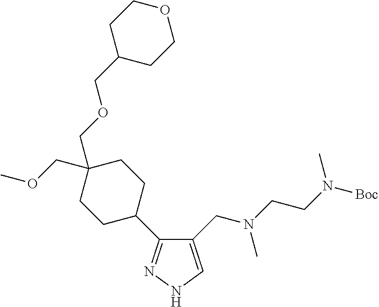 [SMILES: COCC1(COCC2CCOCC2)CCC(c2n[nH]cc2CN(C)CCN(C)C(=O)OC(C)(C)C)CC1]

A solution of tert-butyl (2-(((3-(4-(methoxymethyl)-4-(((tetrahydro-2H-pyran-4 yl) methoxy) methyl)cyclohex-1-en-1-yl)-1H-pyrazol-4-yl)methyl) (methyl) amino) ethyl)(methyl)carbamate (22 mg, 0.042 mmol) in dry THF (5 ml) was hydrogenated under a pressure of 1 atm in the presence of palladium hydroxide (22 mg, 20% on charcoal) for 4 h at rt. The reaction mixture was filtered through Celite and the filtrate was evaporated to obtain a colorless viscous residue which was purified by prep HPLC using X Bridge C18 column (250×19 mm) and 0.1% TFA in water, 100% acetonitrile mobile phase to afford titled compound as colorless viscous mass (81.8%). ESI-MS: m/z=523.97 [M+1].

### Step-10: Synthesis of N1-((3-(4-(methoxymethyl)-4-(((tetrahydro-2H-pyran-4-yl)methoxy)methyl)cyclohexyl)-1H-pyrazol-4-yl)methyl)-N1,N2-dimethylethane-1,2-diamine_HCl Salt

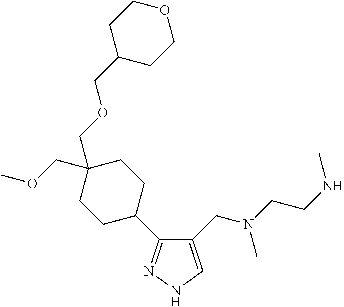 [SMILES: CNCCN(C)Cc1c[nH]nc1C1CCC(COC)(COCC2CCOCC2)CC1]

To a solution of tert-butyl (2-(((3-(4-(methoxymethyl)-4-(((tetrahydro-2H-pyran-4-yl) methoxy) methyl)cyclohexyl)-1H-pyrazol-4-yl) methyl) (methyl) amino)ethyl)(methyl)carbamate (18 mg, 0.034 mmol) in DCM (1 ml) at 0° C. was added trifluroacetic acid. The reaction mixture was stirred for 4 h at rt. The reaction was concentrated under vacuum and triturated with diethyl ether to get the title compound as a white solid compound as the TFA salt (64.2%). ESI-MS: m/z=423.75 [M+1]. ¹H NMR (400 MHz, D₂O) δ: 7.63 (s, 1H), 4.19 (s, 2H), 3.86-3.83 (m, 2H), 3.47-3.15 (m, 15H), 2.67-2.65 (m, 6H), 1.85-1.63 (s, 2H), 1.56 (m, 7H), 1.20-1.14 (m, 5H).

### Example B-31. Synthesis of N1-((3-((6s,9s)-2-oxaspiro[5.5]undecan-9-yl)-1H-pyrazol-4-yl)methyl)-N1,N2-dimethylethane-1,2-diamine (Compound 4)

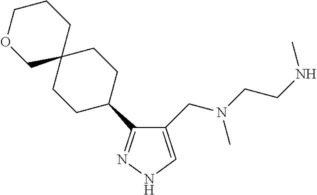 [SMILES: CNCCN(C)Cc1c[nH]nc1[C@H]1CC[C@]2(CCCOC2)CC1]

### Step-1: Synthesis of (3-bromopropoxy)(tert-butyl)dimethylsilane

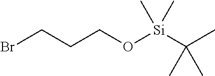

Into a 500 ml round-bottom flask purged and maintained with an inert atmosphere of nitrogen, was placed N,N-dimethylformamide (300 ml), 3-bromopropan-1-ol (30 g, 215.84 mmol, 1.21 equiv.), tert-butyl(chloro)dimethylsilane (26.8 g, 177.81 mmol, 1.00 equiv.), 4H-imidazole (36.4 g, 534.69 mmol, 3.01 equiv.). The resulting solution was stirred for 12 h at 25° C. The resulting solution was diluted with 500 ml of ethyl acetate. The resulting mixture was washed with 3×500 ml of brine. The mixture was dried over anhydrous sodium sulfate and concentrated under vacuum. The residue was applied onto a silica gel column with ethyl acetate. This resulted in 12.4 g of the title compound as colorless oil (28%). ¹H NMR (300 MHz, CDCl₃) δ: 3.76-3.73 (m, 2H), 3.67-3.62 (m, 1H), 3.51 (t, J=6.6 Hz, 1H), 2.07-1.91 (m, 2H), 0.87 (s, 9H), 0.07 (s, 6H).

### Step-2: Synthesis of ethyl 8-[3-[(tert-butyldimethylsilyl)oxy]propyl]-1,4-dioxaspiro[4.5]decane-8-carboxylate

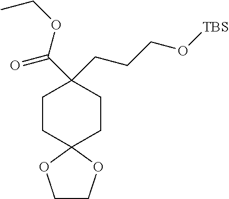

Into a 250 ml 3-necked round-bottom flask purged and maintained with an inert atmosphere of nitrogen, was placed tetrahydrofuran (100 ml). This was followed by the addition of LDA (7.2 g, 1.50 equiv.) dropwise with stirring at −60° C. To the mixture was added a solution of ethyl 1,4-dioxaspiro[4.5]decane-8-carboxylate (12.4 g, 57.87 mmol, 1.54 equiv.) and HMPA (4 g) in tetrahydrofuran (20 ml) dropwise with stirring for 1 h. To this was added a solution of (3-bromopropoxy)(tert-butyl)dimethylsilane (9.5 g, 37.51 mmol, 1.00 equiv.) in tetrahydrofuran (20 ml) dropwise at −60° C. The resulting solution was stirred for 4 h at rt. The resulting solution was diluted with 200 ml of ethyl acetate. The resulting mixture was washed with 3×200 ml of brine. The resulting mixture was concentrated under vacuum. The residue was applied onto a silica gel column with petroleum ether. This resulted in 8 g of the title compound as colorless oil (55%). ¹H NMR (300 MHz, MeOD) δ: 4.19-4.10 (m, 2H), 3.92 (s, 4H), 3.60 (t, J=6.0 Hz, 2H), 2.14-2.09 (m, 2H), 1.80-1.38 (m, 10H), 1.28-1.22 (m, 3H), 0.90 (s, 9H), 0.06 (s, 6H).

### Step-3: Synthesis of (8-[3-[(tert-butyldimethylsilyl)oxy]propyl]-1,4-dioxaspiro[4.5]decan-8-yl)methanol

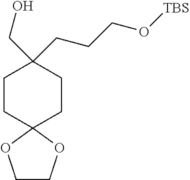

Into a 100 ml 3-necked round-bottom flask purged and maintained with an inert atmosphere of nitrogen, was placed tetrahydrofuran (40 ml), LAH (1.2 g, 31.58 mmol, 1.53 equiv.). This was followed by the addition of a solution of ethyl 8-[3-[(tert-butyldimethylsilyl)oxy]propyl]-1,4-dioxaspiro[4.5]decane-8-carboxylate (8 g, 20.69 mmol, 1.00 equiv.) in tetrahydrofuran (10 ml) dropwise with stirring at −10° C. The resulting solution was stirred for 2 hr at −10° C. The reaction was then quenched by the addition of 10 g of Na₂SO₄.10H₂O. The solids were filtered out. The resulting mixture was concentrated under vacuum. The residue was applied onto a silica gel column with ethyl acetate/petroleum ether (1:2). This resulted in 4.2 g of (8-[3-[(tert-butyldimethylsilyl)oxy]propyl]-1,4-dioxaspiro[4.5]decan-8-yl)methanol as light yellow oil (59%). ¹H NMR (300 MHz, CDCl₃) δ: 3.87 (s, 4H), 3.54 (t, J=6.0 Hz, 2H), 3.38 (s, 2H), 1.57-1.51 (m, 4H), 1.49-1.31 (m, 8H), 0.87 (s, 9H), 0.00 (s, 6H).

### Step-4: Synthesis of 3-[8-(hydroxymethyl)-1,4-dioxaspiro[4.5]decan-8-yl]propan-1-ol

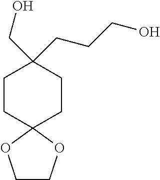 [SMILES: OCCCC1(CO)CCC2(CC1)OCCO2]

Into a 50-ml round-bottom flask purged and maintained with an inert atmosphere of nitrogen, was placed tetrahydrofuran (20 ml), (8-[3-[(tert-butyldimethylsilyl)oxy]propyl]-1,4-dioxaspiro[4.5]decan-8-yl)methanol (4 g, 11.61 mmol, 1.00 equiv.), TBAF (3 g, 11.47 mmol, 0.99 equiv.) was added by batchwise with stirring at 0° C. The resulting solution was stirred for 12 h at room temperature. The resulting mixture was concentrated under vacuum. The residue was applied onto a silica gel column with ethyl acetate. This resulted in 2 g of 3 the title compound as light yellow oil (75%). ¹H NMR (300 MHz, CDCl₃) δ: 3.93 (s, 4H), 3.68-3.59 (m, 2H), 3.43 (s, 2H), 1.72-1.58 (m, 4H), 1.55-1.48 (m, 8H).

### Step-5: Synthesis of 2-oxaspiro[5.5]undecan-9-one ethylene ketal

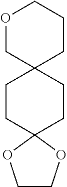 [SMILES: C1COCC2(C1)CCC1(CC2)OCCO1]

Into a 50 ml round-bottom flask purged and maintained with an inert atmosphere of nitrogen, was placed tetrahydrofuran (10 ml), 3-[8-(hydroxymethyl)-1,4-dioxaspiro[4.5]decan-8-yl]propan-1-ol (2 g, 8.68 mmol, 1.00 equiv), n-Bu₃P (1.5 g). TMAD (1.2 g) was added at 0° C. The resulting solution was stirred for 16 h at room temperature. The solids were filtered out. The resulting mixture was concentrated under vacuum. The residue was applied onto a silica gel column with ethyl acetate/petroleum ether (1:20). This resulted in 1.7 g of the title compound as light yellow oil (92%). ¹H NMR (300 MHz, CDCl₃) δ: 3.95 (s, 4H), 3.66-3.60 (m, 2H), 3.43 (s, 2H), 1.72-1.39 (m, 12H).

### Step-6: Synthesis of 2-oxaspiro[5.5]undecan-9-one

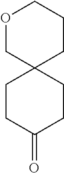 [SMILES: O=C1CCC2(CCCOC2)CC1]

Into a 50 ml round-bottom flask purged and maintained with an inert atmosphere of nitrogen, was placed dichloromethane (20 ml), 2-oxaspiro[5.5]undecan-9-one ethylene ketal (1.7 g, 8.01 mmol, 1.00 equiv.), FeCl₃.6H₂O (4.3 g). The resulting solution was stirred for 12 h at room temperature. The resulting mixture was washed with 1×20 ml of H₂O. The resulting mixture was washed with 3×20 ml of sodium bicarbonate (sat.). The resulting mixture was concentrated under vacuum. The residue was applied onto a silica gel column with ethyl acetate/petroleum ether (1:10). This resulted in 0.9 g of the title compound as a light yellow oil (67%). ¹H NMR (300 MHz, CDCl₃) δ: 3.73-3.66 (m, 2H), 3.50 (s, 2H), 2.35-2.28 (m, 4H), 1.81-1.69 (m, 4H), 1.65-1.59 (m, 4H).

### Step-7: Synthesis of 2-oxaspiro[5.5]undec-8-en-9-yl trifluoromethanesulfonate

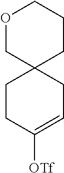 [SMILES: O=S(=O)(OC1=CCC2(CCCOC2)CC1)C(F)(F)F]

Into a 100 ml 3-necked round-bottom flask purged and maintained with an inert atmosphere of nitrogen, was placed tetrahydrofuran (20 ml), 2-oxaspiro[5.5]undecan-9-one (2.7 g, 16.05 mmol, 1.00 equiv.). This was followed by the addition of LiHMDS (1M in THF, 20 ml) dropwise with stirring at −60° C. The resulting solution was stirred for 0.5 h at −20° C. To this was added a solution of Tf₂NPh (5.6 g, 15.82 mmol, 0.99 equiv.) in tetrahydrofuran (10 ml) dropwise with stirring at −60° C. in 30 min. The resulting solution was stirred for 1 h at 25° C. The reaction was then quenched by the addition of 1 ml of water. The resulting mixture was concentrated under vacuum. The resulting solution was diluted with 30 ml of n-hexane. The mixture was dried over anhydrous sodium sulfate. The solids were filtered out. This resulted in 3.5 g of the title compound as light yellow oil (73%). ¹H NMR (300 MHz, CDCl₃) δ: 5.70-5.67 (m, 1H), 3.70-3.55 (m, 2H) 3.40-3.34 (m, 2H), 2.35-2.30 (m, 2H), 2.19-1.97 (m, 2H), 1.63-1.55 (m, 4H), 1.53-1.48 (m, 2H).

### Step-8: Synthesis of 4,4,5,5-tetramethyl-2-[2-oxaspiro[5.5]undec-8-en-9-yl]-1,3,2-dioxaborolane

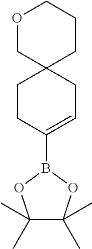

Into a 100 ml 3-round-bottom flask purged and maintained with an inert atmosphere of nitrogen, was placed dioxane (30 ml), 2-oxaspiro[5.5]undec-8-en-9-yl trifluoromethanesulfonate (3.5 g, 11.66 mmol, 1.00 equiv.), Bpin2 (4.4 g), Pd(dppf)₂Cl₂.DCM (850 mg), KOAc (3.4 g). The resulting solution was stirred for 12 h at 80° C. The resulting mixture was concentrated under vacuum. The residue was applied onto a silica gel column with ethyl acetate/petroleum ether (1:20). This resulted in 2.6 g of the title compound as light yellow oil (80%). ¹H NMR (300 MHz, CDCl₃) δ: 6.49 (s, 1H), 3.70-3.50 (m, 2H), 3.40-3.25 (m, 2H), 2.12-2.0 (m, 2H), 1.70-1.52 (m, 4H), 1.48-1.43 (m, 4H), 1.25 (s, 12H).

### Step-9: Synthesis of tert-butyl (2-(((3-(2-oxaspiro[5.5]undec-8-en-9-yl)-1-(tetrahydro-2H-pyran-2-yl)-1H-pyrazol-4-yl)methyl)(methyl)amino)ethyl)(methyl)carbamate

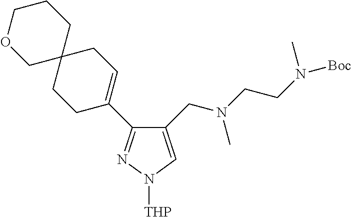

Into a 100 ml 3-necked round-bottom flask purged and maintained with an inert atmosphere of nitrogen, was placed dioxane (50 ml), 4,4,5,5-tetramethyl-2-[2-oxaspiro[5.5]undec-8-en-9-yl]-1,3,2-dioxaborolane (3.6 g, 12.94 mmol, 1.11 equiv.), Pd(dppf)₂Cl₂.DCM (860 mg, 1.18 mmol, 0.10 equiv.), K₂CO₃(4.8 g, 34.78 mmol, 2.97 equiv.), tert-butyl N-[2-([3-iodo-1-(oxan-2-yl)-1H-pyrazol-4-yl]methyl(methyl)amino)ethyl]-N-methylcarbamate (5.6 g, 11.71 mmol, 1.00 equiv.), water (5 ml). The resulting solution was stirred for 12 h at 100° C. The reaction mixture was cooled. The resulting mixture was concentrated under vacuum. The residue was applied onto a silica gel column with ethyl acetate/petroleum ether (1:1). This resulted in 2.6 g the title compound as colorless oil (44%). LCMS: 1.00 min, m/z=503.3 [M+1].

### Step-10: Synthesis of tert-butyl (2-(((3-(2-oxaspiro[5.5]undec-8-en-9-yl)-1H-pyrazol-4-yl)methyl)(methyl)amino)ethyl)(methyl)carbamate

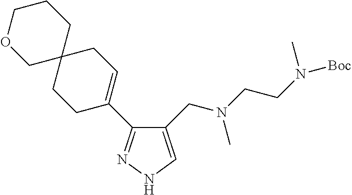 [SMILES: CN(CCN(C)C(=O)OC(C)(C)C)Cc1c[nH]nc1C1=CCC2(CCCOC2)CC1]

Into a 50 ml round-bottom flask, was placed hydrogen chloride (conc.) (0.1 ml), methanol (10 ml), tert-butyl N-methyl-N-[2-[methyl([[1-(oxan-2-yl)-3-[2-oxaspiro[5.5]undec-8-en-9-yl]-1H-pyrazol-4-yl]methyl])amino]ethyl]carbamate (400 mg, 0.80 mmol, 1.00 equiv.). The resulting solution was stirring for 2 h at 25° C. The pH value of the solution was adjusted to 8 with sodium carbonate (sat.). The resulting mixture was concentrated under vacuum. The residue was applied onto a silica gel column with ethyl acetate/petroleum ether (1:1). This resulted in 220 mg of the title compound as light yellow oil (66%). LCMS: 1.17 min, m/z=419.2 [M+1].

### Step-11: Synthesis of tert-butyl (2-(((3-(2-oxaspiro[5.5]undecan-9-yl)-1H-pyrazol-4-yl)methyl)(methyl)amino)ethyl)(methyl)carbamate

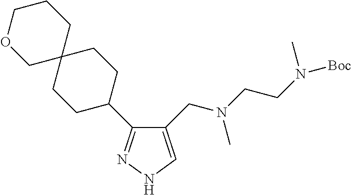 [SMILES: CN(CCN(C)C(=O)OC(C)(C)C)Cc1c[nH]nc1C1CCC2(CCCOC2)CC1]

Into a 25 ml round-bottom flask, was placed tetrahydrofuran (10 ml), tert-butyl N-methyl-N-(2-[methyl[(3-[2-oxaspiro[5.5]undec-8-en-9-yl]-1H-pyrazol-4-yl)methyl]amino]ethyl)carbamate (200 mg, 0.48 mmol, 1.00 equiv.), 10% Pd(OH)₂/C (400 mg). To the above hydrogen was introduced in and maintained at 2 atm pressure. The resulting solution was stirred for 2 h at room temperature. The solids were filtered out. The resulting mixture was concentrated under vacuum. This resulted in 200 mg of the title compound as light yellow oil (100%). LCMS: 1.22 min, m/z=421.5 [M+1].

### Step-12: Synthesis of N1-((3-((6s,9s)-2-oxaspiro[5.5]undecan-9-yl)-1H-pyrazol-4-yl)methyl)-N1,N2-dimethylethane-1,2-diamine

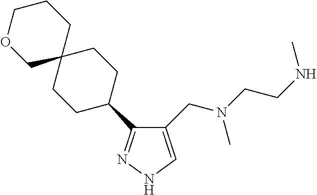 [SMILES: CNCCN(C)Cc1c[nH]nc1[C@H]1CC[C@]2(CCCOC2)CC1]

Into a 25 ml round-bottom flask, was placed dichloromethane (5 ml), tert-butyl N-methyl-N-(2-[methyl[(3-[2-oxaspiro[5.5]undecan-9-yl]-1H-pyrazol-4-yl)methyl]amino]ethyl)carbamate (200 mg, 0.48 mmol, 1.00 equiv.), trifluoroacetic acid (5 ml). The resulting solution was stirred for 0.5 h at 25° C. The resulting mixture was concentrated under vacuum. The crude product was purified by Prep-HPLC with the following conditions (Prep-HPLC-025): Column, XBridge Prep Phenyl OBD Column, 5 um, 19*150 mm; mobile phase, Water with 10 mmol NH₄HCO₃ and MeCN (20.0% MeCN up to 30.0% in 10 min, up to 95.0% in 1 min, hold 95.0% in 1 min, down to 20.0% in 2 min); Detector, UV 220 nm. This resulted in 23.7 mg of the title compound as a yellow solid (16%). LCMS: 1.04 min, m/z=321.2 [M+1]. ¹H NMR (300 MHz, MeOD) δ: 6.92 (s, 1H), 3.3.65-3.62 (m, 2H), 3.39-3.27 (m, 4H), 3.05-3.01 (m, 2H), 2.78-2.54 (m, 6H), 2.12 (s, 3H), 1.81-1.68 (m, 6H), 1.62-1.51 (m, 4H), 1.20-1.00 (m, 2H).

### Example B-32. Synthesis of N1-((3-((6r,9r)-2-oxaspiro[5.5]undecan-9-yl)-1H-pyrazol-4-yl)methyl)-N1,N2-dimethylethane-1,2-diamine (Compound 5)

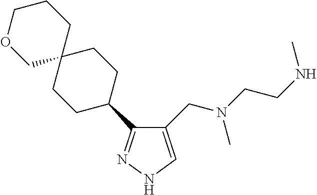 [SMILES: CNCCN(C)Cc1c[nH]nc1[C@H]1CC[C@@]2(CCCOC2)CC1]

### Step-1; Synthesis of N1-((3-((6r,9r)-2-oxaspiro[5.5]undecan-9-yl)-1H-pyrazol-4-yl)methyl)-N1,N2-dimethylethane-1,2-diamine

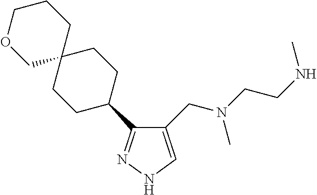 [SMILES: CNCCN(C)Cc1c[nH]nc1[C@H]1CC[C@@]2(CCCOC2)CC1]

Into a 25 ml round-bottom flask, was placed dichloromethane (5 ml), trifluoroacetic acid (5 ml), tert-butyl N-methyl-N-(2-[methyl[(3-[2-oxaspiro[5.5]undecan-9-yl]-1H-pyrazol-4-yl)methyl]amino] ethyl)carbamate (200 mg, 0.48 mmol, 1.00 equiv.). The resulting solution was stirred for 0.5 h at rt. The resulting mixture was concentrated under vacuum. The crude product was purified by Prep-HPLC with the following conditions (Prep-HPLC-025): Column, XBridge Prep Phenyl OBD Column, 5 um, 19*150 mm; mobile phase, Water with 10 mmol NH₄HCO₃ and MeCN (20.0% MeCN up to 30.0% in 10 min, up to 95.0% in 1 min, hold 95.0% in 1 min, down to 20.0% in 2 min); Detector, UV 220 nm. This resulted in 15.5 mg of the title compound as a white solid (10%). LCMS: 1.06 min, m/z=321.1 [M+1]. ¹H NMR (300 MHz, MeOD) δ: 7.64 (s, 1H), 4.05 (s, 2H), 3.36-3.63 (m, 4H), 3.40-3.30 (m, 2H), 3.21-3.18 (m, 2H), 2.83-2.78 (m, 4H), 2.70 (s, 3H), 1.95-1.91 (m, 2H), 1.74-1.61 (m, 6H), 1.47-1.42 (m, 2H), 1.35-1.20 (m, 2H).

### Example B-33. Synthesis of N1-((3-(9-methoxyspiro[5.5]undecan-3-yl)-1H-pyrazol-4-yl)methyl)-N1,N2-dimethylethane-1,2-diamine (Compound 9)

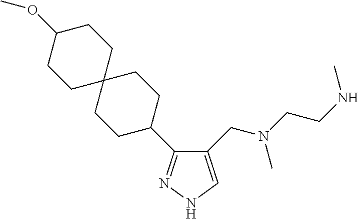 [SMILES: CNCCN(C)Cc1c[nH]nc1C1CCC2(CCC(OC)CC2)CC1]

### Step-1: Synthesis of 1,4-dioxaspiro[4.5]decan-8-ol

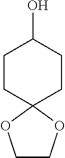 [SMILES: OC1CCC2(CC1)OCCO2]

Into a 500 ml 3-necked round-bottom flask purged and maintained with an inert atmosphere of nitrogen, was placed 1,4-dioxaspiro[4.5]decan-8-one (20 g, 128.06 mmol, 1.00 equiv.), methanol (200 ml). Then NaBH₄ (3.9 g, 105.91 mmol, 0.83 equiv.) was added at 0° C. The resulting solution was stirred at room temperature for 16 h. The reaction was then quenched by the addition of 100 ml of NH₄Cl (sat. aq.). The resulting mixture was concentrated under vacuum. The resulting solution was diluted with 100 ml of H₂O. The resulting solution was extracted with 3×200 ml of dichloromethane and the organic layers combined and dried over anhydrous sodium sulfate and concentrated under vacuum. The residue was applied onto a silica gel column with ethyl acetate/petroleum ether (1:15). This resulted in 16.84 g of the title compound as a colorless oil (83%). ¹H NMR (300 MHz, CDCl₃) δ: 4.01-3.89 (m, 4H), 3.88-3.76 (m, 1H), 1.95-1.74 (m, 4H), 1.74-1.52 (m, 4H).

### Step-2: Synthesis of 8-methoxy-1,4-dioxaspiro[4.5]decane

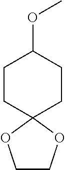

Into a 250 ml 3-necked round-bottom flask purged and maintained with an inert atmosphere of nitrogen, was placed sodium hydride (3.8 g, 95.00 mmol, 1.50 equiv.), N,N-dimethylformamide (100 ml). To the above was added 1,4-dioxaspiro[4.5]decan-8-ol (10 g, 63.21 mmol, 1.00 equiv.) in THF (20 ml) dropwise at 0° C., stirred for 1 h at 50° C., cooled down, was added iodomethane (10.8 g, 76.09 mmol, 1.20 equiv.) dropwise at 0° C. The resulting solution was stirred at 50° C. for 16 h. The reaction was then quenched by the addition of water (100 ml). The resulting solution was extracted with 3×100 ml of ethyl acetate and the organic layers combined. The resulting mixture was washed with 3×100 ml of brine (sat.). The mixture was dried over anhydrous sodium sulfate and concentrated under vacuum. This resulted in 10.8 g of the title compound as a light yellow oil (99%). ¹H NMR (300 MHz, CDCl₃) δ: 4.00-3.90 (m, 4H), 3.30 (s, 3H), 3.30-3.25 (m, 1H), 1.90-1.49 (m, 8H).

### Step-3: Synthesis of 4-methoxycyclohexan-1-one

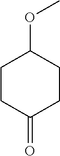

Into a 500 ml 3-necked round-bottom flask, was placed 8-methoxy-1,4-dioxaspiro[4.5]decane (10.8 g, 62.71 mmol, 1.00 equiv.), dichloromethane (200 ml), FeCl₃.6H₂O (50.9 g). The resulting solution was stirred for 3 h at room temperature. The reaction was then quenched by the addition of 200 ml of sodium bicarbonate (sat. aq.). The resulting solution was extracted with 5×200 ml of dichloromethane and the organic layers combined. The resulting mixture was washed with 3×200 ml of brine (sat.). The mixture was dried over anhydrous sodium sulfate and concentrated under vacuum. The residue was applied onto a silica gel column with ethyl acetate/petroleum ether (1:10). This resulted in 3.7 g of the title compound as a yellow oil (46%). ¹H NMR (300 MHz, CDCl₃) δ: 3.61-3.50 (m, 1H), 3.34 (s, 3H), 2.60-2.40 (m, 2H), 2.30-2.15 (m, 2H), 2.15-1.96 (m, 2H), 1.96-1.79 (m, 2H).

### Step-4: Synthesis of 1-methoxy-4-(methoxymethylidene)cyclohexane

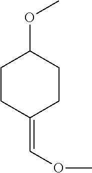

Into a 250 ml 3-necked round-bottom flask purged and maintained with an inert atmosphere of nitrogen, was placed chloro(methoxymethyl)triphenylphosphorane (10.8 g, 31.51 mmol, 1.50 equiv.), tetrahydrofuran (30 ml), to the above was added NaHMDS (1M/L in THF, 32 ml) dropwise at −10° C., stirred for 1 h at −10° C., then was added 4-methoxycyclohexan-1-one (2.7 g, 21.07 mmol, 1.00 equiv.) in THF dropwise at −10° C. The resulting solution was stirred for 3 h at −10° C. The reaction was then quenched by the addition of 30 ml of NH₄Cl (sat. aq.). The resulting mixture was concentrated under vacuum. The resulting solution was extracted with 3×50 ml of ethyl acetate and the organic layers combined and dried over anhydrous sodium sulfate and concentrated under vacuum. The residue was applied onto a silica gel column with ethyl acetate/petroleum ether (1:10). This resulted in 1.33 g of the title compound as a light yellow oil (40%). ¹H NMR (300 MHz, CDCl₃) δ: 5.76 (s, 1H), 3.54 (s, 3H), 3.40-3.20 (m, 4H), 2.70-2.50 (m, 1H), 2.19-2.02 (m, 1H), 1.93-1.80 (m, 4H), 1.48-1.30 (m, 2H).

### Step-5: Synthesis of 4-methoxycyclohexane-1-carbaldehyde

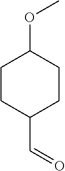

Into a 100 mL round-bottom flask, was placed 1-methoxy-4-(methoxymethylidene)cyclohexane (1.33 g, 8.51 mmol, 1.00 equiv.), formic acid (10 ml). The resulting solution was stirred for 1 h at 90° C. The resulting mixture was concentrated under vacuum. The resulting solution was diluted with 20 ml of H₂O. The resulting solution was extracted with 3×30 ml of ethyl acetate and the organic layers combined and dried over anhydrous sodium sulfate and concentrated under vacuum. The residue was applied onto a silica gel column with ethyl acetate/petroleum ether (1:15). This resulted in 0.8 g of 4-methoxycyclohexane-1-carbaldehyde as yellow oil (66%).

### Step-6: Synthesis of 9-methoxyspiro[5.5]undec-1-en-3-one

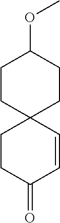

Into a 100 ml round-bottom flask, was placed 4-methoxycyclohexane-1-carbaldehyde (800 mg, 5.63 mmol, 1.00 equiv.), benzene (8 ml), but-3-en-2-one (0.4 g 1.00 equiv.), sulfuric acid (0.05 ml, 98%). The mixture was stirred for 1.5 h at 45° C., then another but-3-en-2-one (0.39 g 1.00 equiv.) was added. The resulting solution was stirred for 2 h at 90° C. The reaction was then quenched by the addition of 20 ml of sodium bicarbonate (sat. aq.). The resulting solution was extracted with 3×50 ml of ethyl acetate and the organic layers combined and dried over anhydrous sodium sulfate and concentrated under vacuum. The residue was applied onto a silica gel column with ethyl acetate/petroleum ether (1:10). This resulted in 0.35 g of the title compound as a yellow oil (32%). LCMS: 1.33 min, m/z=195.0 [M+1].

### Step-7: Synthesis of 9-methoxyspiro[5.5]undecan-3-one

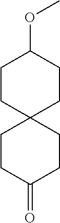

Into a 50 ml round-bottom flask, was placed 9-methoxyspiro[5.5]undec-1-en-3-one (400 mg, 2.06 mmol, 1.00 equiv.), methanol (10 ml), 10% Palladium carbon (0.05 g). To the above hydrogen was introduced in and maintained at 2 atm pressure. The resulting solution was stirred for 1 h at room temperature. The solids were filtered out. The resulting mixture was concentrated under vacuum. This resulted in 0.4 g of the title compound as a colorless oil (99%). ¹H NMR (300 MHz, CDCl₃) δ: 3.35 (s, 3H), 3.31-3.18 (m, 1H), 2.40-2.29 (m, 4H), 1.90-1.61 (m, 8H), 1.56-1.40 (m, 2H), 1.39-1.22 (m, 2H).

### Step-8: Synthesis of 9-methoxyspiro[5.5]undec-2-en-3-yl trifluoromethanesulfonate

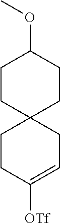 [SMILES: COC1CCC2(CC=C(OS(=O)(=O)C(F)(F)F)CC2)CC1]

Into a 50 ml 3-necked round-bottom flask purged and maintained with an inert atmosphere of nitrogen, was placed 9-methoxyspiro[5.5]undecan-3-one (400 mg, 2.04 mmol, 1.00 equiv.), tetrahydrofuran (6 ml), to the above was added LiHMDS (1M/L in THF, 3.1 ml) dropwise at −78 to −40° C., stirred for 1 h at −40° C. To the above was added 1,1,1-trifluoro-N-phenyl-N-(trifluoromethane)sulfonylmethanesulfonamide (800 mg, 2.24 mmol, 1.10 equiv.) by dropwise. The resulting solution was stirred for 3 h at room temperature. The reaction was then quenched by the addition of 5 ml of water. The resulting mixture was concentrated under vacuum. The residue was applied onto a silica gel column with ethyl acetate/petroleum ether (1:20). This resulted in 0.45 g (67%) of the title compound as a colorless oil. ¹H NMR (300 MHz, CDCl₃) δ: 5.67 (s, 1H), 3.33 (s, 3H), 3.29-3.10 (m, 1H), 2.42-2.20 (m, 2H), 2.11-2.01 (m, 1H), 2.00-1.92 (m, 1H), 1.88-1.72 (m, 2H), 1.72-1.62 (m, 1H), 1.62-1.51 (m, 3H), 1.51-1.39 (m, 2H), 1.28-1.15 (m, 2H).

### Step-9: Synthesis of 2-[9-methoxyspiro[5.5]undec-2-en-3-yl]-4,4,5,5-tetramethyl-1,3,2-dioxaborolane

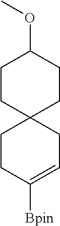

Into a 100 ml round-bottom flask purged and maintained with an inert atmosphere of nitrogen, was placed 9-methoxyspiro[5.5]undec-2-en-3-yl trifluoromethanesulfonate (450 mg, 1.37 mmol, 1.00 equiv.), 4,4,5,5-tetramethyl-2-(tetramethyl-1,3,2-dioxaborolan-2-yl)-1,3,2-dioxaborolane (418 mg, 1.65 mmol, 1.20 equiv.), Pd(dppf)Cl₂ (100 mg, 0.14 mmol, 0.10 equiv.), potassium acetate (403 mg, 4.11 mmol, 3.00 equiv.), 1,4-dioxane (10 ml). The resulting solution was stirred at 80° C. for 16 h in an oil bath. The residue was dissolved in 20 ml of water. The resulting solution was extracted with 3×20 ml of ethyl acetate and the organic layers combined. The resulting mixture was washed with 1×50 ml of brine. The mixture was dried over anhydrous sodium sulfate. The solids were filtered out. The resulting mixture was concentrated under vacuum. The residue was applied onto a silica gel column with ethyl acetate/petroleum ether (1:20). This resulted in 250 mg of the title compound as a yellow oil (60%). ¹H NMR (300 MHz, CDCl₃) δ: 6.47 (s, 1H), 3.33 (s, 3H), 3.25-3.10 (m, 1H), 2.20-2.17 (m, 2H), 2.05-1.95 (m, 1H), 1.84-1.91 (m, 1H), 1.85-1.68 (m, 2H), 1.53-1.32 (m, 5H), 1.30-1.22 (m, 12H), 1.19-1.05 (m, 3H).

### Step-10: Synthesis of tert-butyl (2-(((3-(9-methoxyspiro[5.5]undec-2-en-3-yl)-1-(tetrahydro-2H-pyran-2-yl)-1H-pyrazol-4-yl)methyl)(methyl)amino)ethyl)(methyl)carbamate

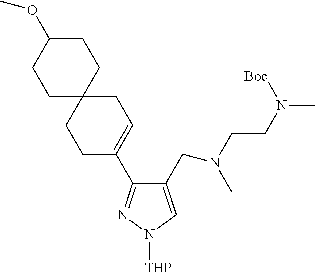

Into a 100 mL round-bottom flask purged and maintained with an inert atmosphere of nitrogen, was placed tert-butyl N-[2-([[3-iodo-1-(oxan-2-yl)-1H-pyrazol-4-yl]methyl](methyl)amino)ethyl]-N-methylcarbamate (392 mg, 0.82 mmol, 1.00 equiv.), 2-[9-methoxyspiro[5.5]undec-2-en-3-yl]-4,4,5,5-tetramethyl-1,3,2-dioxaborolane (250 mg, 0.82 mmol, 1.00 equiv.), Pd(dppf)Cl₂ (120 mg, 0.16 mmol, 0.20 equiv.), potassium methaneperoxoate (339 mg, 2.44 mmol, 2.97 equiv.), water (2 ml), 1,4-dioxane (20 ml). The resulting solution was stirred at 100° C. for 16 h in an oil bath. The resulting mixture was concentrated under vacuum. The residue was applied onto a silica gel column with ethyl acetate/petroleum ether (1:1). This resulted in 260 mg of the title compound as a brown oil (60%). LCMS: 1.38 min, m/z=531.5 [M+1].

### Step-11: Synthesis of tert-butyl (2-(((3-(9-methoxyspiro[5.5]undecan-3-yl)-1-(tetrahydro-2H-pyran-2-yl)-1H-pyrazol-4-yl)methyl)(methyl)amino)ethyl)(methyl)carbamate

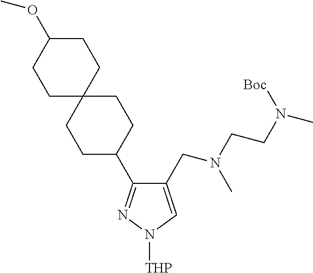

Into a 100 ml round-bottom flask, was placed tert-butyl N-(2-[[(3-[9-methoxyspiro[5.5]undec-2-en-3-yl]-1-(oxan-2-yl)-1H-pyrazol-4-yl)methyl](methyl)amino]ethyl)-N-methylcarbamate (260 mg, 0.49 mmol, 1.00 equiv.), 10% Pd(OH)₂/C (520 mg), tetrahydrofuran (15 ml). To the above hydrogen was introduced in and maintained at 2 atm pressure. The resulting solution was stirred for 2 h at room temperature. The solids were filtered out. The resulting mixture was concentrated under vacuum. This resulted in 210 mg of the title compound as a brown oil (80%). LCMS: 1.39 min, m/z=533.6 [M+1].

### Step-12: Synthesis of N1-((3-(9-methoxyspiro[5.5]undecan-3-yl)-1H-pyrazol-4-yl)methyl)-N1,N2-dimethylethane-1,2-diamine

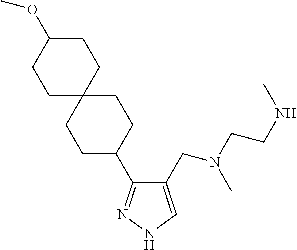 [SMILES: CNCCN(C)Cc1c[nH]nc1C1CCC2(CCC(OC)CC2)CC1]

Into a 50-mL round-bottom flask, was placed tert-butyl N-(2-[[(3-[9-methoxyspiro[5.5]undecan-3-yl]-1-(oxan-2-yl)-1H-pyrazol-4-yl)methyl](methyl)amino]ethyl)-N-methylcarbamate (210 mg, 0.39 mmol, 1.00 equiv.), trifluoroacetic acid (5 ml), dichloromethane (5 ml). The resulting solution was stirred for 20 min at room temperature. The resulting solution was extracted with 10 ml of water, and the aqueous layer was washed with 3×10 ml of DCM. The resulting mixture was concentrated under vacuum. The crude product was purified by Prep-HPLC with the following conditions (Waters): Column, Atlantis Prep OBD T3 Column, 19×150 mm, 5 um; mobile phase, water with 0.05% trifluoroacetic acid and CH₃CN (up to 3.0% in 10 min, up to 100.0% in 1 min, hold 100.0% in 1 min); Detector, UV 220 nm. This resulted in 173.8 mg of the title compound as the bis TFA salt as colorless oil (76%). LCMS: 1.18 min, m/z=349.3 [M−2TFA+1]. ¹H NMR (300 MHz, D₂O) δ: 7.75 (s, 1H), 4.29 (s, 2H), 3.45 (s, 4H), 3.26 (s, 4H), 2.76 (s, 3H), 2.71 (s, 4H), 1.98-1.80 (m, 2H), 1.80-1.65 (m, 2H), 1.65-1.42 (m, 5H), 1.40-1.02 (m, 7H).

### Example B-34. Synthesis of (5r,8r)-2-isopentyl-8-(4-((methyl(2-(methylamino)ethyl)amino)methyl)-1H-pyrazol-3-yl)-2-azaspiro[4.5]decan-1-one (Compound 11)

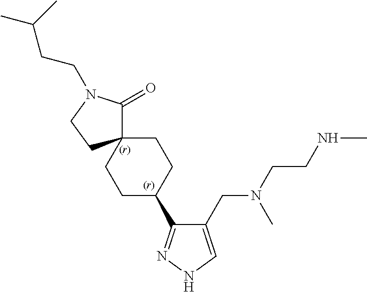 [SMILES: CNCCN(C)Cc1c[nH]nc1[C@H]1CC[C@@]2(CCN(CCC(C)C)C2=O)CC1]

### Step-1: Synthesis of ethyl 1,4-dioxaspiro[4.5]decane-8-carboxylate

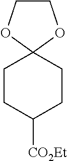 [SMILES: CCOC(=O)C1CCC2(CC1)OCCO2]

Into a 1000 ml round-bottom flask, was placed cyclohexane (400 ml), ethyl 4-oxocyclohexane-1-carboxylate (215 g, 1.26 mol, 1.00 equiv.), ethane-1,2-diol (94.1 g, 1.52 mol, 1.20 equiv.), sulfamic acid (5.0 g, 51.50 mmol, 0.04 equiv.). The resulting solution was stirred to water segregator for 2 h at 100° C. in an oil bath. The reaction was then quenched by the addition of 800 mL of water. The resulting solution was extracted with 3×800 ml of ethyl acetate and the organic layers combined and dried over anhydrous sodium sulfate and concentrated under vacuum. The residue was applied onto a silica gel column with ethyl acetate/petroleum ether (1:100-1:30). This resulted in 242 g of the title compound as light yellow oil (89%). ¹H NMR (300 MHz, CDCl₃) δ: 4.09-4.02 (m, 2H), 3.87 (s, 4H), 2.30-2.23 (m, 1H), 1.90-1.85 (m, 2H), 1.80-1.67 (m, 4H), 1.53-1.48 (m, 2H), 1.20 (t, J=7.2 Hz, 3H).

### Step-2: Synthesis of ethyl 8-(cyanomethyl)-1,4-dioxaspiro[4.5]decane-8-carboxylate

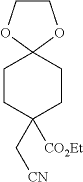 [SMILES: CCOC(=O)C1(CC#N)CCC2(CC1)OCCO2]

Into a 1000 ml 3-necked round-bottom flask purged and maintained with an inert atmosphere of N₂, this was added THF (90 ml), diisopropylamine (41.36 g, 0.41 mol), n-BuLi (156 ml, 0.39 mol, 2.5 M in hexane) at −40 to −30° C. in a liquid nitrogen bath. The resulting solution was stirred for 0.5 h −40 to −30° C. To this was added a solution of ethyl 1,4-dioxaspiro[4.5]decane-8-carboxylate (60 g, 280.04 mmol, 1.00 equiv.) in tetrahydrofuran (150 ml) dropwise with stirring. The resulting solution was stirred for 0.5 h at −78 to −50° C. To the mixture was added a solution of BrCH₂CN (40.37 g, 336.44 mmol, 1.20 equiv.) in tetrahydrofuran (100 ml) dropwise with stirring. The resulting solution was stirred for 1 h at −78 to −50° C. The reaction was then quenched by the addition of 1000 ml of NH₄Cl (sat. aq.). The solids were filtered out. The resulting solution was extracted with 3×1000 ml of ethyl acetate and the organic layers combined. The resulting mixture was washed with 1×2000 ml of brine (sat.). The resulting mixture was concentrated under vacuum. The residue was applied onto a silica gel column with ethyl acetate/petroleum ether (1:5-1:3). This resulted in 26.8 g of the title compound as a red oil (38%). ¹H NMR (300 MHz, CDCl₃) δ: 4.20-4.13 (m, 2H), 3.87 (s, 4H), 2.52 (s, 2H), 2.21-2.14 (m, 2H), 1.80-1.65 (m, 6H), 1.30 (t, J=7.2 Hz, 3H). LCMS: 0.91 min, m/z=254.0 [M+1].

### Step-3: Synthesis of 2-azaspiro[4.5]decane-1,8-dione ethylene ketal

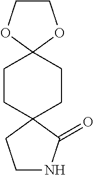 [SMILES: O=C1NCCC12CCC1(CC2)OCCO1]

Into a 500 ml round-bottom flask, was placed ethanol (300 ml), ethyl 8-(cyanomethyl)-1,4-dioxaspiro[4.5]decane-8-carboxylate (26.8 g, 105.81 mmol, 1.00 equiv.), Raney Ni (3.0 g). To the above H₂ was introduced in and maintained at 2 atm pressure. The resulting solution was stirred for 2 d at 50° C. in an oil bath. The solids were filtered out. The resulting mixture was concentrated under vacuum. The residue was applied onto a silica gel column with dichloromethane/methanol (10:1). This resulted in 2.64 g (12%) of the title compound as a white solid. ¹H NMR (400 MHz, CDCl₃) δ: 5.86 (s, 1H), 3.88 (s, 4H), 3.25 (t, J=6.8 Hz, 2H), 1.98 (t, J=6.8 Hz, 2H), 1.93-1.89 (m, 2H), 1.82-1.78 (m, 2H), 1.56-1.52 (m, 2H), 1.49-1.44 (m, 2H). LCMS: 0.89, m/z=211.9 [M+1].

### Step-4: Synthesis of 2-isopentyl-2-azaspiro[4.5]decane-1,8-dione ethylene ketal

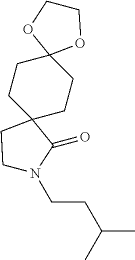 [SMILES: CC(C)CCN1CCC2(CCC3(CC2)OCCO3)C1=O]

Into a 100 ml 3-necked round-bottom flask purged and maintained with an inert atmosphere of nitrogen, was placed N,N-dimethylformamide (25 ml), 2-azaspiro[4.5]decane-1,8-dione ethylene ketal (2.64 g, 12.50 mmol, 1.00 equiv.). Then sodium hydride (751 mg, 31.29 mmol, 60%, 2.50 equiv.) was added at 0° C. by batchwise. The resulting solution was stirred for 0.5 h in an ice/salt bath. This was followed by the addition of 1-iodo-3-methylbutane (4.95 g, 24.99 mmol, 2.00 equiv.) dropwise with stirring at 0° C. The resulting solution was stirred for 16 h at room temperature. The reaction was then quenched by the addition of 100 ml of water. The resulting solution was extracted with 3×100 ml of ethyl acetate and the organic layers combined. The resulting mixture was washed with 1×100 ml of brine (sat.). The mixture was dried over anhydrous sodium sulfate and concentrated under vacuum. The residue was applied onto a silica gel column with ethyl acetate/petroleum ether (1:100-1:50). This resulted in 3.12 g of the title compound as a white solid (89%). ¹H NMR (300 MHz, CDCl₃) δ: 3.88 (s, 4H), 3.24-3.17 (m, 4H), 1.91-1.76 (m, 6H), 1.56-1.30 (m, 7H), 0.84 (d, J=6.6 Hz, 6H). LCMS: 1.14 min, m/z=282.1 [M+1].

### Step-5: Synthesis of 2-(3-methylbutyl)-2-azaspiro[4.5]decane-1,8-dione

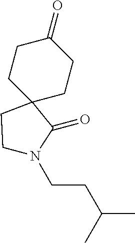 [SMILES: CC(C)CCN1CCC2(CCC(=O)CC2)C1=O]

Into a 100 ml round-bottom flask, was placed dichloromethane (40 ml), 2-isopentyl-2-azaspiro[4.5]decane-1,8-dione ethylene ketal (3.12 g, 11.09 mmol, 1.00 equiv.), FeCl₃-6H₂O (9.0 g). The resulting solution was stirred for 16 h at room temperature. The reaction was then quenched by the addition of 100 ml of sodium bicarbonate (sat. aq.). The resulting solution was extracted with 3×100 ml of ethyl acetate and the organic layers combined and dried over anhydrous sodium sulfate and concentrated under vacuum. The residue was applied onto a silica gel column with ethyl acetate/petroleum ether (1:50-1:20). This resulted in 640 mg of the title compound as a colorless oil (24%). ¹H NMR (300 MHz, CDCl₃) δ: 3.32-3.20 (m, 4H), 2.70-2.61 (m, 2H), 2.31-2.21 (m, 2H), 2.13-2.03 (m, 2H), 1.98 (t, J=6.9 Hz, 2H), 1.76-1.71 (m, 2H), 1.49-1.45 (m, 1H), 1.38-1.31 (m, 2H), 0.86 (d, J=6.3 Hz, 6H). LCMS: 1.35, m/z=238.1[M+1].

### Step-6: Synthesis of 2-(3-methylbutyl)-1-oxo-2-azaspiro[4.5]dec-7-en-8-yl trifluoromethanesulfonate

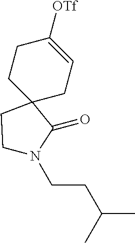 [SMILES: CC(C)CCN1CCC2(CC=C(OS(=O)(=O)C(F)(F)F)CC2)C1=O]

Into a 50 ml 3-necked round-bottom flask purged and maintained with an inert atmosphere of nitrogen, was placed tetrahydrofuran, 2-(3-methylbutyl)-2-azaspiro[4.5]decane-1,8-dione (640 mg, 2.70 mmol, 1.00 equiv.), THF (6 ml), LiHMDS (3.24 ml, 1.0 M/L in THF) at −78° C. The resulting solution was stirred at −78° C. for 1 h in a liquid nitrogen bath. Then the solution of 1,1,1-trifluoro-N-phenyl-N-(trifluoromethane) sulfonylmethanesulfonamide (1060 mg, 2.97 mmol, 1.10 equiv.) in 2 ml THF was dropped at −78° C. The resulting solution was stirred at rt for 16 h. The reaction was then quenched by the addition of 20 ml of NH₄Cl (sat. aq.). The resulting solution was extracted with 3×20 ml of ethyl acetate and the organic layers combined and dried over anhydrous sodium sulfate and concentrated under vacuum. The residue was applied onto a silica gel column with ethyl acetate/petroleum ether (1:50-1:20). This resulted in 720 mg of the title compound as a light yellow oil (72%). LCMS: 1.68 min, m/z=370.2 [M+1]. ¹H NMR (300 MHz, CDCl₃) δ: 5.69 (t, J=1.8 Hz, 1H), 3.28-3.22 (m, 4H), 2.53-2.46 (m, 1H), 2.39-2.35 (m, 2H), 2.03-1.93 (m, 2H), 1.86-1.81 (m, 2H), 1.59-1.40 (m, 2H), 1.37-1.30 (m, 2H), 0.85 (d, J=6.6 Hz, 6H).

### Step-7: Synthesis of 2-(3-methylbutyl)-8-(tetramethyl-1,3,2-dioxaborolan-2-yl)-2-azaspiro[4.5]dec-7-en-1-one

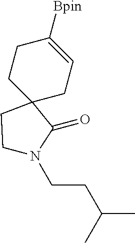

Into a 50 ml round-bottom flask purged and maintained with an inert atmosphere of nitrogen, was placed dioxane (7 ml), 2-(3-methylbutyl)-1-oxo-2-azaspiro[4.5]dec-7-en-8-yl trifluoromethanesulfonate (720 mg, 1.95 mmol, 1.00 equiv.), 4,4,5,5-tetramethyl-2-(tetramethyl-1,3,2-dioxaborolan-2-yl)-1,3,2-dioxaborolane (595 mg, 2.34 mmol, 1.20 equiv.), Pd(dppf)Cl₂—CH₂Cl₂ (159.4 mg, 0.20 mmol, 0.10 equiv.) and KOAc (573.7 mg, 5.85 mmol, 3.00 equiv.). The resulting solution was stirred for 16 h at 80° C. in an oil bath. The resulting mixture was concentrated under vacuum. The residue was applied onto a silica gel column with ethyl acetate/petroleum ether (1:20-1:10). This resulted in 660 mg of the title compound as a light yellow oil (97%). LCMS: 1.02 min, m/z=222.0 [M+1]. ¹H NMR (300 MHz, CDCl₃) δ: 6.46 (t, J=1.8 Hz, 1H), 3.25-3.18 (m, 4H), 2.45-2.20 (m, 2H), 2.15-1.95 (m, 1H), 1.85-1.70 (m, 4H), 1.57-1.28 (m, 4H), 1.15 (s, 12H), 0.85 (d, J=6.6 Hz, 6H).

### Step-8: Synthesis of 3-[2-(3-methylbutyl)-1-oxo-2-azaspiro[4.5]dec-7-en-8-yl]-1-(oxan-2-yl)-1H-pyrazole-4-carbaldehyde

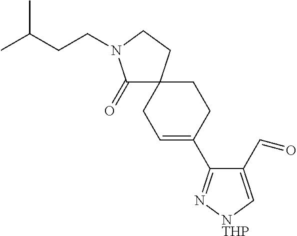

Into a 50 ml round-bottom flask purged and maintained with an inert atmosphere of nitrogen, was placed dioxane (5 ml), water (0.5 ml), 3-iodo-1-(oxan-2-yl)-1H-pyrazole-4-carbaldehyde (489 mg, 1.60 mmol, 1.00 equiv.), 2-(3-methylbutyl)-8-(tetramethyl-1,3,2-dioxaborolan-2-yl)-2-azaspiro[4.5]dec-7-en-1-one (610 mg, 1.76 mmol, 1.10 equiv.), Pd(dppf)Cl₂—CH₂Cl₂ (130.6 mg, 0.16 mmol, 0.10 equiv.), potassium carbonate (441 mg, 3.19 mmol, 2.00 equiv.). The resulting solution was stirred for 16 h at 100° C. in an oil bath. The reaction was then quenched by the addition of 20 ml of water/ice. The resulting solution was extracted with 3×30 ml of ethyl acetate and the organic layers combined and dried over anhydrous sodium sulfate and concentrated under vacuum. The residue was applied onto a silica gel column with ethyl acetate/petroleum ether (1:2-1:1). This resulted in 380 mg of the title compound as a light yellow oil (55%). LCMS: 1.56 min, m/z=400.3 [M+1]. ¹H NMR (300 MHz, CDCl₃) δ: 9.85 (s, 1H), 8.07 (s, 1H), 6.28-6.27 (m, 1H), 5.31-5.23 (m, 1H), 4.06-4.00 (m, 1H), 3.65-3.64 (m, 1H), 3.28-3.23 (m, 4H), 2.70-2.40 (m, 3H), 2.03-1.80 (m, 6H), 1.75-1.50 (m, 6H), 1.38-1.21 (m, 2H), 0.86 (d, J=6.3 Hz, 6H).

### Step-9: Synthesis of tert-butyl (2-(((3-(2-isopentyl-1-oxo-2-azaspiro[4.5]dec-7-en-8-yl)-1-(tetrahydro-2H-pyran-2-yl)-1H-pyrazol-4-yl)methyl)(methyl)amino)ethyl)(methyl)carbamate

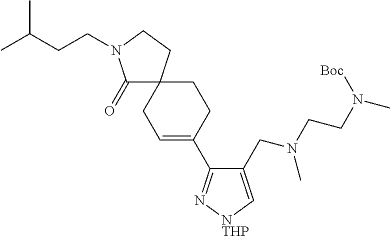

Into a 50 ml round-bottom flask, was placed DCE (5 ml), 3-[2-(3-methylbutyl)-1-oxo-2-azaspiro[4.5]dec-7-en-8-yl]-1-(oxan-2-yl)-1H-pyrazole-4-carbaldehyde (380 mg, 0.95 mmol, 1.00 equiv.), tert-butyl N-methyl-N-[2-(methylamino)ethyl]carbamate (197 mg, 1.05 mmol, 1.10 equiv.). Then NaBH(OAc)₃ (606 mg, 2.86 mmol, 3.01 equiv.) was added into by batchwise. The resulting solution was stirred for 8 h at room temperature. The reaction was then quenched by the addition of 15 ml of sodium bicarbonate (sat. aq.). The resulting solution was extracted with 3×15 ml of dichloromethane and the organic layers combined and dried over anhydrous sodium sulfate and concentrated under vacuum. The residue was applied onto a silica gel column with ethyl acetate/petroleum ether (1:2˜1:1). This resulted in 440 mg of the title compound as a light yellow oil (81%). LCMS: 1.38 min, m/z=572.5[M+1]. ¹H NMR (300 MHz, CD₃OD) δ: 7.45 (s, 1H), 6.15 (s, 1H), 5.23 (s, 1H), 4.02-3.97 (m, 1H), 3.64-3.58 (m, 1H), 3.40-3.26 (m, 4H), 3.25 (s, 3H), 2.76 (s, 3H), 2.62-2.35 (m, 5H), 2.16 (m, 2H), 1.98-1.81 (m, 7H), 1.65-1.45 (m, 7H), 1.37 (s, 9H), 1.33-1.30 (m, 2H),0.86 (d, J=6.6 Hz, 6H).

### Step-10: Synthesis of tert-butyl (2-(((3-(2-isopentyl-1-oxo-2-azaspiro[4.5]decan-8-yl)-1-(tetrahydro-2H-pyran-2-yl)-1H-pyrazol-4-yl)methyl)(methyl)amino)ethyl) (methyl)carbamate

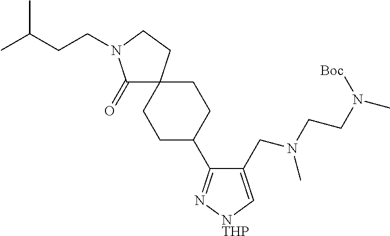

Into a 20 ml pressure tank reactor, was placed acetic acid (4 ml), tert-butyl N-methyl-N-[2-[methyl([3-[2-(3-methylbutyl)-1-oxo-2-azaspiro[4.5]dec-7-en-8-yl]-1-(oxan-2-yl)-1H-pyrazol-4-yl]methyl)amino]ethyl]carbamate (440 mg, 0.77 mmol, 1.00 equiv.), 10% Palladium carbon (100 mg). To the above H₂ was introduced in and maintained at 2 atm pressure. The resulting solution was stirred 16 h at room temperature. The solids were filtered out. The resulting mixture was concentrated under vacuum. This resulted in 550 mg of the title compound as a yellow oil that was used in the next step without further purification. LCMS: 1.47 min, m/z=390.3 [M+1].

### Step-11: Synthesis of (5r,8r)-2-isopentyl-8-(4-((methyl(2-(methylamino)ethyl)amino)methyl)-1H-pyrazol-3-yl)-2-azaspiro[4.5]decan-1-one

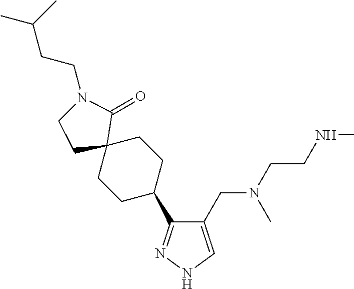 [SMILES: CNCCN(C)Cc1c[nH]nc1[C@H]1CC[C@@]2(CCN(CCC(C)C)C2=O)CC1]

Into a 50 ml round-bottom flask, was placed tert-butyl N-methyl-N-[2-[methyl([3-[2-(3-methylbutyl)-1-oxo-2-azaspiro[4.5]decan-8-yl]-1-(oxan-2-yl)-1H-pyrazol-4-yl]methyl)amino]ethyl]carbamate (300 mg, 0.52 mmol, 1.00 equiv.), dichloromethane (5 ml), trifluoroacetic acid (5 ml). The resulting solution was stirred for 16 h at rt. The resulting mixture was concentrated under vacuum. The crude product was purified by Prep-HPLC with the following conditions: Column: X Bridge C18, 19×50 mm, 5 um; Mobile Phase A: water/10 mmol NH₄HCO₃, Mobile Phase B: ACN; Flow rate: 20 ml/min; Gradient: 30% B to 70% B in 10 min; 254 nm. This resulted in 121.1 mg of the title compound as a colorless oil (59%). LCMS: 1.47 min, m/z=390.3[M+1]. ¹H NMR (300 MHz, CD₃OD) δ: 7.45 (s, 1H), 3.49 (s, 2H), 3.40-3.34 (m, 3H), 3.31-3.29 (m, 1H), 2.93 (t, J=6.0 Hz, 2H), 2.88-2.77 (m, 1H), 2.60 (t, J=6.0 Hz, 2H), 2.53 (s, 3H), 2.36-2.30 (m, 2H), 2.25 (s, 3H), 2.05-1.95 (m, 2H), 1.89 (t, J=6.6 Hz, 2H), 1.75-1.65 (m, 2H), 1.65-1.40 (m, 5H), 0.95 (d, J=6.3 Hz, 6H).

### Example B-35. Synthesis of (5s,8s)-3,3-diethyl-8-(4-((methyl(2-(methylamino)ethyl)amino)methyl)-1H-pyrazol-3-yl)-1-oxaspiro[4.5]decan-2-one and (5r,8r)-3,3-diethyl-8-(4-((methyl(2-(methylamino)ethyl)amino)methyl)-1H-pyrazol-3-yl)-1-oxaspiro[4.5]decan-2-one (Compound 33 and 34)

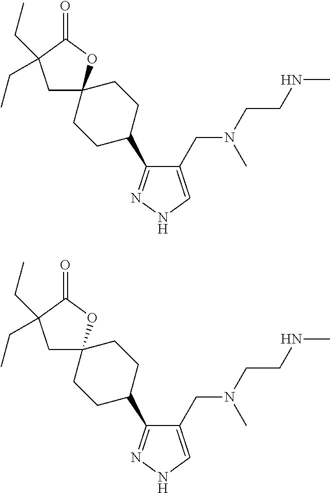

### Step-1: Synthesis of 1,7,10-trioxadispiro[2.2.46.23]dodecane

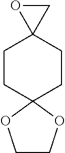 [SMILES: C1COC2(CCC3(CC2)CO3)O1]

To a stirred suspension of trimethyl sulfonium iodide (31.38 g, 153 mmol) in DMF (150 ml), sodium hydride (60% in mineral oil) (6.65 g, 277 mmol) was added under inert condition at 0° C. The reaction mixture was stirred for 30 min. at 0° C. followed by the addition of 1,4-dioxaspiro[4.5]decan-8-one (20 g, 128 mmol) in DMF (50 ml) dropwise. The reaction mixture was allowed to stirred at rt for 30 min at which time the reaction mixture was poured into ice-cold water slowly and extracted with ethyl acetate (3×100 ml), and the combined organic layers were collected and dried over sodium sulphate and concentrated under vacuum to dryness. The residue was purified by silica gel column chromatography using mobile phase (0%-10%) ethyl acetate in hexane to afford the title compound as pale light yellow liquid (92.6%). LCMS: m/z=171.1 [M+1]. ¹H NMR (400 MHz, CDCl₃) δ: 4.01-3.94 (m, 4H), 2.69 (s, 2H), 2.01-1.90 (m, 4H) 1.82-1.73 (m, 2H), 1.66-1.53 (m, 2H).

### Step-2: Synthesis of tert-butyl 3-(8-hydroxy-1,4-dioxaspiro[4.5]decan-8-yl)propanoate

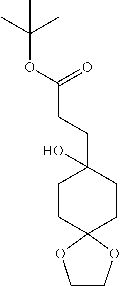 [SMILES: CC(C)(C)OC(=O)CCC1(O)CCC2(CC1)OCCO2]

A solution of diisopropylamine (11.60 g, 114.6 mmol) in dry THF (130 ml) was cooled to −78° C. followed by dropwise addition of n-butyl lithium (1.6M in hexane) under nitrogen atmosphere. The reaction mixture was brought to 0° C. for 30 min and again cooled to −78° C., then t-butyl acetate was added dropwise and stirred for a further 30 min at −78° C. Diethyl aluminum chloride (1M in hexane, 105.8 ml, 105.8 mmol) was then added and the resulting mixture was stirred for 30 min at −78° C. A solution of 1,7,10-trioxadispiro [2.2.46.23]dodecane (15 g, 88.2 mmol) in THF (90 ml) was added slowly added to reaction mass at −78° C. and the reaction mixture was stirred at −60 to −50° C. for 6 h. The reaction was quenched with saturated NH₄Cl solution and extracted with ethyl acetate (3×30 ml). The organic layer was collected and dried over sodium sulphate and concentrated in vacuum to dryness. The residue was purified by silica gel column chromatography using mobile phase (0%-40%) ethyl acetate in hexane to obtain the title compound as a pale yellow liquid (56%). LCMS: m/z=287.1 [M+1].

### Step-3: Synthesis of 1,4,9-trioxadispiro[4.2.48.25]tetradecan-10-one

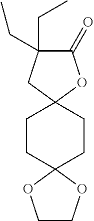 [SMILES: CCC1(CC)CC2(CCC3(CC2)OCCO3)OC1=O]

To a solution of 1,7,10-trioxadispiro[2.2.46.23]dodecane (2.8 g, 9.7 mmol) in chloroform (50 ml) was added p-toluene sulphonic acid (0.074 g, 0.38 mmol) at rt. The reaction mixture was heated up to 70° C. for 2 h. The reaction was poured into water (25 ml), neutralized with sodium bicarbonate solution and extracted with ethyl acetate (3×25 ml), the organic layer was dried over sodium sulphate and concentrated in vacuum to dryness. The residue was purified by silica gel column chromatography using mobile phase (0%-30%) ethyl acetate in hexane to obtain the title compound (81%). ESI-MS: m/z=264.0 [M+1]. ¹H NMR (400 MHz, CDCl₃) δ: 4.01-3.94 (m, 4H), 2.65-2.61 (t, 2H), 2.09-2.05 (t, 2H), 1.99-1.92 (m, 4H), 1.88-1.80 (m, 2H), 1.70-1.67 (m, 2H).

### Step-4: Synthesis of 11,11-diethyl-1,4,9-trioxadispiro[4.2.48.25]tetradecan-10-one

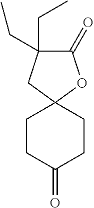 [SMILES: CCC1(CC)CC2(CCC(=O)CC2)OC1=O]

A solution of diisopropylamine (16.31 g, 160.8 mmol) in THF (160 ml) was cooled to −78° C. followed by dropwise addition of n-butyl lithium (1.6M in hexane, 100.5 ml, 160.8 mmol). The reaction mixture was brought to 0° C. for 30 min and again cool to −78° C. followed by addition of 1,4,9-trioxadispiro [4.2.48.25] tetradecan-10-one (5.7 g, 26.8 mmol) in THF (40 ml). The reaction mixture was stirred for additional 30 min at −78° C. and ethyl iodide (25.14 g, 160.8 mmol) and the reaction mixture was allowed to warm to rt and stir for 4 h. The reaction was quenched with sat. NH₄Cl solution and extracted with ethyl acetate (3×100 ml), the organic layer was collected and dried over sodium sulphate and concentrated in vacuum to dryness. The residue was purified by silica gel column chromatography using mobile phase (0%-30%) ethyl acetate in hexane to obtain the title compound (20.8%). ESI-MS: m/z=269.3 [M+1].

### Step 5: Synthesis of 3,3-diethyl-1-oxaspiro[4.5]decane-2,8-dione

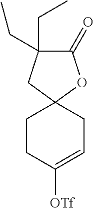 [SMILES: CCC1(CC)CC2(CC=C(OS(=O)(=O)C(F)(F)F)CC2)OC1=O]

To a solution of 11,11-diethyl-1,4,9-trioxadispiro[4.2.48.25]tetradecan-10-one (1.4 g, 5.2 mmol) in THF (12 ml) was added 3N HCl solution (8 ml) at 0° C. The reaction mixture was stirred for 24 h and then diluted with water, neutralized with aq. sodium bicarbonate solution and extracted with ethyl acetate. The organic layer was dried and evaporated to obtain the crude which was purified by silica gel column chromatography using mobile phase (0%-30%) ethyl acetate in hexane to obtain the title compound (88.8%). ESI-MS: m/z=225.3 [M+1].

### Step-6: Synthesis of 3,3-diethyl-2-oxo-1-oxaspiro[4.5]dec-7-en-8-yl trifluoromethanesulfonate

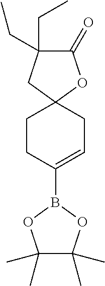 [SMILES: CCC1(CC)CC2(CC=C(B3OC(C)(C)C(C)(C)O3)CC2)OC1=O]

To a stirred solution of 3,3-diethyl-1-oxaspiro[4.5]decane-2,8-dione (0.480 g, 2.1 mmol) in tetrahydrofuran (6 ml) was added in LiHMDS (1M in THF, 2.57 ml) −78° C. and stirred for 45 min. 1,1,1-trifluoro-N-phenyl-N-(trifluoromethane)sulfonylmethane sulfonamide (0.765 g, 2.1 mmol) was added into the reaction mixture and stirred at rt for 1 h. The reaction was then quenched by the addition of 15 ml of sat. NH₄Cl (aq.). The resulting solution was extracted with 3×20 ml of ethyl acetate and the organic layer was dried and evaporated. The resulting crude was dissolved in ethylene glycol (20 ml) and extracted with hexane and the organic layer was concentrated under vacuum to get desired compound as light yellow oil that was used in the next step without further purification. ESI-MS: m/z=357.2 [M+1].

### Step-7: Synthesis of 3,3-diethyl-8-(4,4,5,5-tetramethyl-1,3,2-dioxaborolan-2-yl)-1-oxaspiro[4.5]dec-7-en-2-one

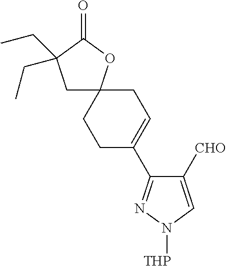

To a stirred solution of 3,3-diethyl-2-oxo-1-oxaspiro[4.5]dec-7-en-8-yl trifluoromethanesulfonate (0.700 g, 1.9 mmol), 4,4,5,5-tetramethyl-2-(tetramethyl-1,3,2-dioxaborolan-2-yl)-1,3,2-dioxaborolane (0.499 g, 1.9 mmol), potassium acetate (0.578 g, 5.8 mmol) in 1,4-dioxane (10 ml) was added Pd(dppf)Cl₂ (0.143 g, 0.19 mmol) under inert atmosphere. The resulting solution was stirred at 80° C. for 1 h. The reaction mixture diluted with water (25 ml), the resulting solution was extracted with (3×25 ml) of ethyl acetate and the organic layers combined. The resulting mixture was washed with (3×20 ml) of brine solution. The mixture was dried over anhydrous sodium sulfate. The residue was purified by silica gel column chromatography using mobile phase (0%-12%) ethyl acetate in hexane to obtain the title compound as yellow oil (56.3%). ESI-MS: m/z=335.43 [M+1].

### Step-8: Synthesis of 3-(3,3-diethyl-2-oxo-1-oxaspiro[4.5]dec-7-en-8-yl)-1-(tetrahydro-2H-pyran-2-yl)-1H-pyrazole-4-carbaldehyde

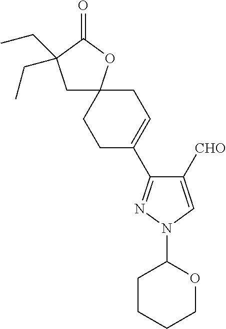 [SMILES: CCC1(CC)CC2(CC=C(c3nn(C4CCCCO4)cc3C=O)CC2)OC1=O]

To a stirred solution of 3,3-diethyl-8-(4,4,5,5-tetramethyl-1,3,2-dioxaborolan-2-yl)-1-oxaspiro[4.5]dec-7-en-2-one (1.48 g, 4.42 mmol) in 1,4-dioxane (17 ml) was added 3-iodo-1-(tetrahydro-2H-pyran-2-yl)-1H-pyrazole-4-carbaldehyde (1.219 g, 3.98 mmol), K₃PO₄ (2.81 g, 13.2 mmol) and water (2 ml) followed by purging with argon gas for 30 min. Pd(dppf)Cl₂ (0.323 g, 0.448 mmol) was added and the resulting solution was stirred for 2 h at 80° C. The reaction mixture was diluted with water (25 ml) and extracted with (3×50 ml) of ethyl acetate and the organic layers combined. The organic layer was washed with (3×50 ml) of brine solution, dried over anhydrous sodium sulfate and concentrated under vacuum. The residue was purified by silica gel column chromatography using mobile phase (0%-30%) ethyl acetate in hexane to afford the title compound (60.8%). LCMS: m/z=303.28 [M+1].

### Step-9: Synthesis of 3-(3,3-diethyl-2-oxo-1-oxaspiro[4.5]dec-7-en-8-yl)-1H-pyrazole-4-carbaldehyde

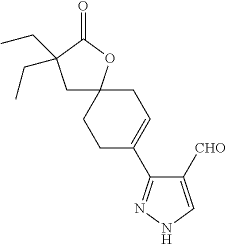 [SMILES: CCC1(CC)CC2(CC=C(c3n[nH]cc3C=O)CC2)OC1=O]

To as solution of 3-(3,3-diethyl-2-oxo-1-oxaspiro[4.5]dec-7-en-8-yl)-1-(tetrahydro-2H-pyran-2-yl)-1H-pyrazole-4-carbaldehyde (1.04 g, 2.69 mmol) in methanol (4 ml) was added concentrated HCl (4 ml) at 0° C. The reaction mixture was stirred at room temperature for 4 h at which time the reaction was neutralized with sodium bicarbonate solution, extracted with dichloromethane (3×25 ml), and the combined organic layers were dried over sodium sulphate and evaporate under vacuum to get desired crude product that was used in the next step without further purification.

### Step 10: Synthesis of tert-butyl (2-(((3-(3,3-diethyl-2-oxo-1-oxaspiro[4.5]dec-7-en-8-yl)-1H-pyrazol-4-yl)methyl)(methyl)amino)ethyl)(methyl)carbamate

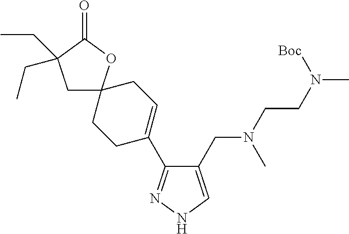 [SMILES: CCC1(CC)CC2(CC=C(c3n[nH]cc3CN(C)CCN(C)C(=O)OC(C)(C)C)CC2)OC1=O]

To a stirred solution of 3-(3,3-diethyl-2-oxo-1-oxaspiro[4.5]dec-7-en-8-yl)-1H-pyrazole-4-carbaldehyde (0.4 g, 1.32 mmol) and tert-butyl methyl(2-(methylamino)ethyl)carbamate (0.299 g, 1.58 mmol) in dry methanol (4 ml) was added with ZnCl₂ (0.009 g, 0.065 mmol) The reaction mixture was stirred at rt for 10 min followed by addition of triethylamine (0.668 g, 6.6 mmol). The reaction mixture was heated to 50° C. for 5 h, sodium cyanoborohydride (0.416 g, 6.7 mmol) was added at 0° C. The reaction mixture was stirred for 16 h at rt. The reaction was diluted with water and product was extracted in ethyl acetate (3×25 ml), the organic layer was collected and dried over sodium sulphate, concentrated in vacuum to dryness. The residue was purified by silica gel column chromatography using mobile phase (0%-4%) MeOH in DCM to give the title compound (45%). LCMS: m/z=475.61 [M+1].

Step 11: Synthesis of tert-butyl (2-(((3-((5s,8s)-3,3-diethyl-2-oxo-1-oxaspiro[4.5]decan-8-yl)-1H-pyrazol-4-yl)methyl)(methyl)amino)ethyl)(methyl)carbamate and tert-butyl (2-(((3-((5r,8r)-3,3-diethyl-2-oxo-1-oxaspiro[4.5]decan-8-yl)-1H-pyrazol-4-yl)methyl)(methyl)amino)ethyl)(methyl)carbamate

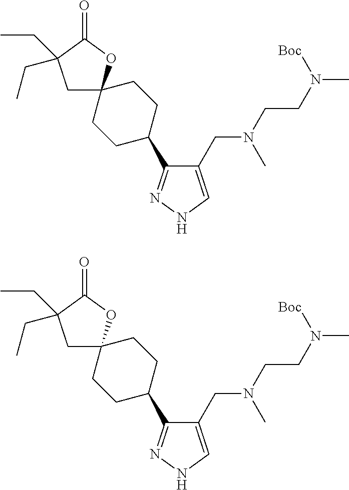

To a stirred suspension of 20% palladium hydroxide on charcoal (0.144 g) in THF (10 ml) under nitrogen atmosphere was added solution of tert-butyl (2-(((3-(3,3-diethyl-2-oxo-1-oxaspiro[4.5]dec-7-en-8-yl)-1H-pyrazol-4-yl)methyl)(methyl)amino)ethyl)(methyl)carbamate (0.284 g, 0.59 mmol) in THF (3 ml). The reaction mixture was purged with hydrogen gas for 2 h and filtered through Celite. The filtrate was concentrated in vacuum to dryness to obtain the title crude compound containing two regioisomers (0.290 g). The isomers were separated by prep-HPLC using X Bridge C18 column (250×19 mm) and 0.1% TFA in water, 100% acetonitrile mobile phase to afford two fractions: Fraction 1 (54 mg), Fraction 2 (120 mg). LCMS: m/z=477.7 [M+1].

### Step-12: Synthesis of (5s,8s)-3,3-diethyl-8-(4-((methyl(2-(methylamino)ethyl)amino)methyl)-1H-pyrazol-3-yl)-1-oxaspiro[4.5]decan-2-one

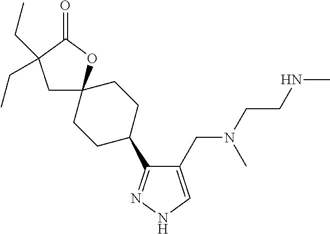 [SMILES: CCC1(CC)C[C@]2(CC[C@H](c3n[nH]cc3CN(C)CCNC)CC2)OC1=O]

tert-butyl (2-(((3-((5s,8s)-3,3-diethyl-2-oxo-1-oxaspiro[4.5]decan-8-yl)-1H-pyrazol-4-yl)methyl)(methyl)amino)ethyl)(methyl)carbamate (0.120 g, 0.26 mmol) and HCl in IPA was stirred for 16 h at rt. The reaction mixture was evaporated under vacuum followed by trituration with diethyl ether to get solid compound which was converted to free base by using polymer supported ammonium carbonate in methanol to get title compound (100%). LCMS: m/z=377.49 [M+1]. ¹H NMR (400 MHz, D₂O) δ: 7.59 (s, 1H), 4.15 (s, 2H), 3.36-3.22 (m, 4H), 2.68 (m, 1H), 2.60 (s, 3H), 2.05 (s, 3H), 1.85-1.80 (m, 4H), 1.56-1.48 (m, 6H), 1.41-1.33 (m, 4H), 0.66-0.63 (t, 6H).

### Step-13: Synthesis of (5r,8r)-3,3-diethyl-8-(4-((methyl(2-(methylamino)ethyl)amino)methyl)-1H-pyrazol-3-yl)-1-oxaspiro[4.5]decan-2-one

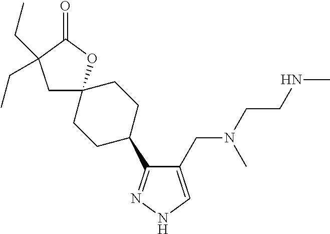 [SMILES: CCC1(CC)C[C@]2(CC[C@@H](c3n[nH]cc3CN(C)CCNC)CC2)OC1=O]

Tert-butyl (2-(((3-((5r,8r)-3,3-diethyl-2-oxo-1-oxaspiro[4.5]decan-8-yl)-1H-pyrazol-4-yl)methyl)(methyl)amino)ethyl)(methyl)carbamate (54 mg, 0.11 mmol) and HCl in IPA was stirred for 16 h at rt. The reaction mixture was evaporated under vacuum followed by trituration with diethyl ether to get solid compound which was converted to free base by using polymer supported ammonium carbonate in methanol to get the title compound. LCMS: m/z=377.49 [M+1]. ¹H NMR (400 MHz, D₂O) δ: 7.60 (s, 1H), 4.16 (s, 2H), 3.39-3.26 (m, 4H), 2.61 (s, 4H), 2.55 (s, 3H), 1.99 (s, 2H), 1.78-1.61 (m, 6H), 1.56-1.39 (m, 6H), 0.68-0.65 (t, 6H).

Table 3 shows the structural data of exemplified compounds.

TABLE 3
Representative Structural Data

| Cmpd | | |
|---|---|---|
| No | Structure | Data |
| 20 | 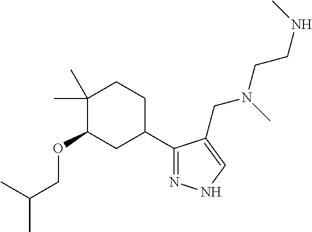 [SMILES: CNCCN(C)Cc1c[nH]nc1C1CCC(C)(C)[C@H](OCC(C)C)C1] | LC/MS: 2.66 min, m/z = 351.10 [M + 1]. ¹H NMR (500 MHz, Methanol-d4) δ: 8.40 (s, 1H), 4.52 (s, 2H), 3.86-3.53 (m, 4H), 3.42 (s, 1H), 3.28 (m, 2H), 2.93 (s, 3H), 2.80 (s, 3H), 2.23-2.04 (m, 1H), 1.97-1.49 (m, 6H), 1.29 (s, 1H), 1.05 (s, 3H), 1.00 (s, 3H), 0.91 (dd, J = 11.9, 6.5 Hz, 6H). |
| 30 | 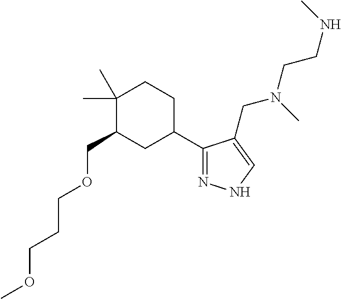 [SMILES: CNCCN(C)Cc1c[nH]nc1C1CCC(C)(C)[C@H](COCCCOC)C1] | LCMS: 5.09 min, m/z = 381.4 [M + 1]. ¹H NMR (500 MHz, Methanol-d4) δ: 7.43 (s, 1H), 3.68-3.39 (m, 7H), 3.29 (s, 3H), 3.13 (t, J = 8.8 Hz, 1H), 2.75 (t, J = 6.4 Hz, 3H), 2.54 (t, J = 6.2 Hz, 2H), 2.41 (s, 3H), 2.20 (s, 3H), 1.96-1.88 (m, 1H), 1.87-1.38 (m, 8H), 1.04 (s, 3H), 0.93 (s, 3H). |
| 25 | 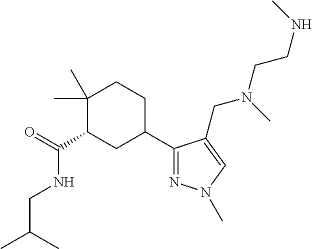 [SMILES: CNCCN(C)Cc1cn(C)nc1C1CCC(C)(C)[C@@H](C(=O)NCC(C)C)C1] | No UV response, 0.91 min, m/z = 392.15 [M + 1]. ¹H NMR (500 MHz, CDCl₃) δ: 7.22 (2s, 1H), 5.67 (m, 1H), 3.83 (2s, 3H), 3.35 (dd, J = 19.2, 3.8 Hz, 2H), 3.18-2.98 (m, 2H), 2.73-2.63 (m, 3H), 2.50 (td, J = 5.9, 2.6 Hz, 2H), 2.43 (2s, 3H), 2.14 (2s, 3H), 2.10-1.94 (m, 2H), 1.93-1.51 (m, 6H), 1.37 (td, J = 13.5, 3.7 Hz, 1H), 1.12 (2s, 3H), 1.06 (2s, 3H), 0.91 (2d, J = 6.6 Hz, 6H). |
| 31 | 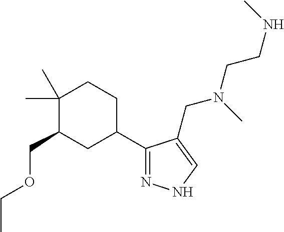 [SMILES: CCOC[C@@H]1CC(c2n[nH]cc2CN(C)CCNC)CCC1(C)C] | LC/MS: 2.28 min, m/z = 337.20 [M + 1]. ¹H NMR (500 MHz, Methanol-d4) δ: 7.42 (s, 1H), 3.69-3.57 (m, 1H), 3.56- 3.37 (m, 4H), 3.12 (t, J = 8.9 Hz, 1H), 2.83-2.71 (m, 1H), 2.67 (t, J = 6.5 Hz, 2H), 2.51 (t, J = 6.1 Hz, 2H), 2.39-2.32 (m, 3H), 2.23-2.15 (m, 3H), 2.00-1.90 (m, 1H), 1.90-1.71 (m, 1H), 1.71-1.60 (m, 1H), 1.60-1.35 (m, 4H), 1.24-1.12 (m, 3H), 1.04 (s, 3H), 0.94 (s, 3H). |
| 32 | 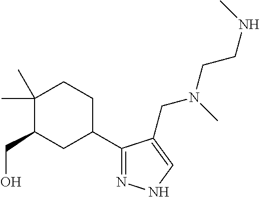 [SMILES: CNCCN(C)Cc1c[nH]nc1C1CCC(C)(C)[C@H](CO)C1] | LCMS: 1.76 min, m/z = 309.10 [M + 1]. ¹H NMR (500 MHz, Methanol-d4) δ: 7.42 (s, 1H), 3.86-3.76 (m, 1H), 3.48- 3.38 (m, 2H), 3.22 (dd, J = 10.6, 8.7 Hz, 1H), 2.76 (ddd, J = 12.2, 8.6, 3.7 Hz, 1H), 2.67 (t, J = 6.5 Hz, 2H), 2.51 (t, J = 6.4 Hz, 2H), 2.36 (s, 3H), 2.27-2.14 (m, 3H), 2.10-1.92 (m, 1H), 1.78 (qd, J = 13.1, 3.8 Hz, 1H), 1.72-1.59 (m, 1H), 1.54-1.38 (m, 4H), 1.09 (s, 3H), 0.93 (s, 3H). |
| 35 | 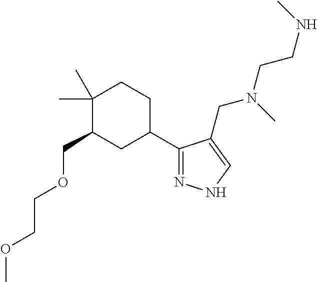 [SMILES: CNCCN(C)Cc1c[nH]nc1C1CCC(C)(C)[C@H](COCCOC)C1] | LCMS: 2.27 min, m/z 367.15 [M + 1]. ¹H NMR (500 MHz, Methanol-d4) δ: 7.42 (s, 1H), 3.70-3.62 (m, 1H), 3.61- 3.54 (m, 1H), 3.54-3.48 (m, 3H), 3.42 (s, 2H), 3.33 (s, 3H), 3.17 (t, J = 9.0 Hz, 1H), 2.76 (tt, J = 12.2, 3.5 Hz, 1H), 2.67 (t, J = 6.6 Hz, 2H), 2.54-2.48 (m, 2H), 2.38-2.34 (m, 3H), 2.22-2.17 (m, 3H), 1.99-1.90 (m, 1H), 1.90-1.72 (m, 1H), 1.71-1.62 (m, 1H), 1.62-1.54 (m, 1H), 1.54-1.33 (m, 3H), 1.04 (s, 3H), 0.94 (s, 3H). |
| 18 | 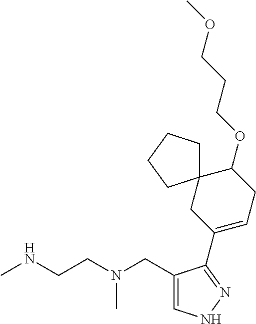 [SMILES: CNCCN(C)Cc1c[nH]nc1C1=CCC(OCCCOC)C2(CCCC2)C1] | LCMS: 2.49 min, m/z = 391.15 [M + 1]. ¹H NMR (500 MHz, Methanol-d₄) δ: 8.59 (s, 0.1H), 8.54-8.49 (m, 0.9H), 6.22 (s, 0.86H), 6.07 (s, 0.14H), 4.56 (s, 2H), 3.78-3.56 (m, 5H), 3.52-3.44 (m, 3H), 3.36 (q, J = 5.6, 4.1 Hz, 1H), 3.33 (d, J = 2.5 Hz, 3H), 2.97-2.92 (m, 0.42H), 2.87 (s, 2.58H), 2.80 (s, 3H), 2.62-2.40 (m, 2H), 2.26 (d, J = 16.8 Hz, 1H), 2.00 (ddd, J = 32.0, 15.1, 9.7 Hz, 1H), 1.81 (qt, J = 10.1, 5.0 Hz, 3H), 1.76-1.63 (m, 4H), 1.65-1.41 (m, 3H). |
| 19 | 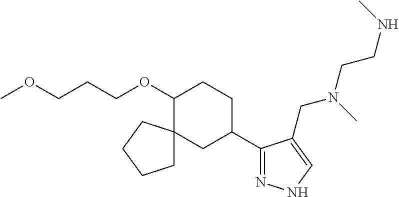 [SMILES: CNCCN(C)Cc1c[nH]nc1C1CCC(OCCCOC)C2(CCCC2)C1] | LCMS: 2.47 min, m/z = 393.2 [M + 1]. ¹H NMR (500 MHz, Methanol-d₄) δ: 7.42 (s, 1H), 3.66 (dt, J = 9.2, 6.0 Hz, 1H), 3.54 (t, J = 6.4 Hz, 2H), 3.42 (s, 2H), 3.40- 3.32 (m, 4H), 3.05 (s, 1H), 2.97-2.87 (m, 1H), 2.73 (t, J = 6.5 Hz, 2H), 2.52 (t, J = 6.5 Hz, 2H), 2.39 (s, 3H), 2.22 (s, 3H), 2.03 (dt, J = 14.1, 3.0 Hz, 1H), 1.92 (t, J = 12.9 Hz, 1H), 1.88-1.76 (m, 4H), 1.70- 1.51 (m, 7H), 1.48-1.32 (m, 3H). |
| 27 | 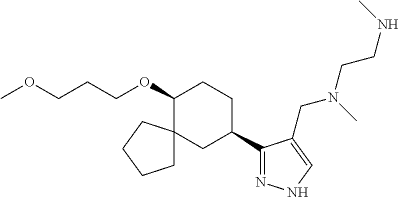 [SMILES: CNCCN(C)Cc1c[nH]nc1[C@@H]1CC[C@H](OCCCOC)C2(CCCC2)C1] | LCMS: 5.03 min, m/z = 393.3 [M + 1]. ¹H NMR (500 MHz, Methanol-d₄) δ: 7.41 (s, 1H), 3.66 (dt, J = 9.2, 6.0 Hz, 1H), 3.55 (t, J = 6.4 Hz, 2H), 3.41 (s, 2H), 3.39- 3.32 (m, 4H), 3.05 (s, 1H), 2.93 (td, J = 12.7, 11.2, 6.5 Hz, 1H), 2.67 (t, J = 6.6 Hz, 2H), 2.50 (t, J = 6.6 Hz, 2H), 2.35 (s, 3H), 2.21 (s, 3H), 2.06-1.98 (m, 1H), 1.96-1.88 (m, 1H), 1.87-1.74 (m, 4H), 1.70-1.49 (m, 7H), 1.48-1.31 (m, 3H). |
| 26 | 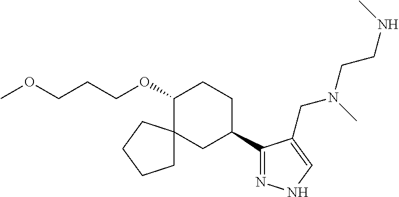 [SMILES: CNCCN(C)Cc1c[nH]nc1[C@@H]1CC[C@@H](OCCCOC)C2(CCCC2)C1] | LCMS: 4.9 min, m/z = 393.3 [M + 1] (also shows 14% of the cis isomer at 5.51 min). ¹H NMR (500 MHz, Methanol-d₄) δ: 7.41 (s, 1H), 3.71 (dt, J = 9.2, 6.0 Hz, 1H), 3.57-3.52 (m, 1H), 3.49 (dtt, J = 9.4, 6.3, 3.0 Hz, 2H), 3.44- 3.36 (m, 3H), 3.33 (s, 3H), 3.18-3.10 (m, 1H), 2.90 (td, J = 11.0, 9.4, 6.4 Hz, 1H), 2.68 (t, J = 6.5 Hz, 2H), 2.56-2.46 (m, 2H), 2.35 (s, 3H), 2.20 (s, 3H), 2.08 (dt, J = 10.7, 3.5 Hz, 1H), 1.96-1.75 (m, 4H), 1.73-1.48 (m, 6H), 1.44 (dt, J = 12.2, 6.1 Hz, 1H), 1.39-1.27 (m, 2H), 1.22 (td, J = 12.4, 11.9, 6.7 Hz, 1H). |
| 127 | 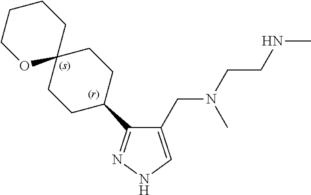 | LCMS: 1.21 min, m/z = 321.1 [M + 1 − 2TFA]. ¹H-NMR (300 MHz, D₂O): δ 8.06 (s, 1H), 4.35 (s, 2H), 3.65-3.35 (m, 6H), 2.99-2.82 (m, 1H), 2.76 (s, 3H), 2.69 (s, 3H), 2.12-1.95 (m, 2H), 1.79-1.20 (m, 12H). |
| 4 | 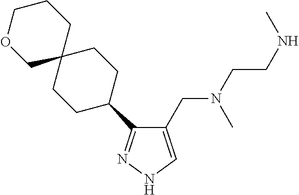 [SMILES: CNCCN(C)Cc1c[nH]nc1[C@H]1CC[C@]2(CCCOC2)CC1] | LCMS: 1.04 min, m/z = 321.2 [M + 1]. ¹H NMR (300 MHz, MeOD): δ 6.92 (s, 1H), 3.65-3.62 (m, 2H), 3.39-3.27 (m, 4H), 3.05- 3.01 (m, 2H), 2.78-2.54 (m, 6H), 2.12 (s, 3H), 1.81-1.68 (m, 6H), 1.62-1.51 (m, 4H), 1.20-1.00 (m, 2H). |
| 5 | 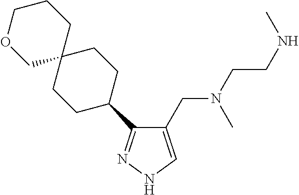 [SMILES: CNCCN(C)Cc1c[nH]nc1[C@H]1CC[C@@]2(CCCOC2)CC1] | LCMS: 1.06 min, m/z = 321.1 [M + 1]. ¹H NMR (300 MHz, MeOD) δ: 7.64 (s, 1H), 4.05 (s, 2H), 3.36-3.63 (m, 4H), 3.40-3.30 (m, 2H), 3.21-3.18 (m, 2H), 2.83-2.78 (m, 4H), 2.70 (s, 3H), 1.95-1.91 (m, 2H), 1.74-1.61 (m, 6H), 1.47-1.42 (m, 2H), 1.35-1.20 (m, 2H). |
| 9 | 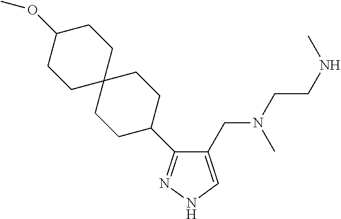 [SMILES: CNCCN(C)Cc1c[nH]nc1C1CCC2(CCC(OC)CC2)CC1] | LCMS: 1.18 min, m/z = 349.3 [M − 2TFA + H]. ¹H NMR (300 MHz, D₂O) δ: 7.75 (s, 1H), 4.29 (s, 2H), 3.45 (s, 4H), 3.26 (s, 4H), 2.76 (s, 3H), 2.71 (s, 4H), 1.98-1.80 (m, 2H), 1.80- 1.65 (m, 2H), 1.65-1.42 (m, 5H), 1.40-1.02 (m, 7H). |
| 21 | 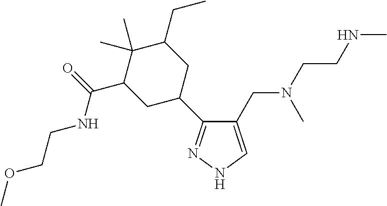 | LCMS: 1.28 min, m/z = 408.0 [M + 1]. ¹H-NMR (300 MHz, D₂O) δ: 7.85-7.70 (m, 1H), 4.35-4.15 (m, 2H), 3.60-3.20 (m, 11H), 2.90-2.70 (m, 7H), 2.65-2.35 (m, 1H), 2.22- 2.15 (m, 0.5H), 2.00-1.45 (m, 4.5H), 1.40-1.10 (m, 2H), 0.95-0.70 (m, 9H). |
| 14 | 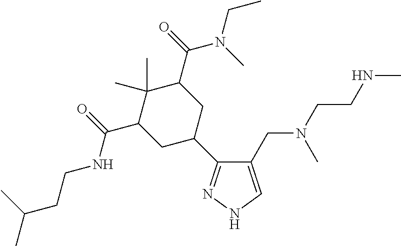 [SMILES: CCN(C)C(=O)C1CC(c2n[nH]cc2CN(C)CCNC)CC(C(=O)NCCC(C)C)C1(C)C] | LCMS: 1.31 min, m/z = 477.3 [M + 1]. ¹H-NMR (400 MHz, D₂O) δ: 7.73 (d, J = 2.4 Hz, 1H), 4.35 (d, J = 2.8 Hz, 2H), 3.82 (t, J = 8.4 Hz, 1H), 3.75-3.35 (m, 5H), 3.25-3.12 (m, 3H), 3.11-3.00 (m, 2H), 2.84-2.81 (m, 5H), 2.70 (s, 3H), 2.20 (d, J = 4.8 Hz, 1H), 1.98- 1.62 (m, 3H), 1.60-1.42 (m, 2H), 1.32 (q, J = 6.8 Hz, 2H), 1.12-1.04 (m, 4H), 0.98 (t, J = 6.8 Hz, 2H), 0.91 (d, J = 3.6 Hz, 3H), 0.79 (dd, J = 6.8 and 1.6 Hz, 6H). F-NMR (400 MHz, D₂O) δ: −75.61. |
| 11 | 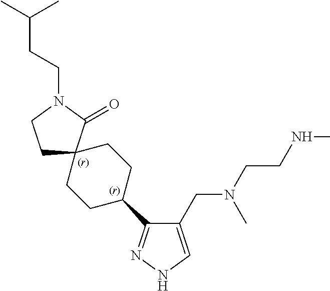 [SMILES: CNCCN(C)Cc1c[nH]nc1[C@H]1CC[C@@]2(CCN(CCC(C)C)C2=O)CC1] | LCMS: 1.47 min, m/z = 390.3 [M + 1]. ¹H NMR (300 MHz, CD₃OD) δ: 7.45 (s, 1H), 3.49 (s, 2H), 3.40-3.34 (m, 3H), 3.31-3.29 (m, 1H), 2.93 (t, J = 6.0 Hz, 2H), 2.88-2.77 (m, 1H), 2.60 (t, J = 6.0 Hz, 2H), 2.53 (s, 3H), 2.36-2.30 (m, 2H), 2.25 (s, 3H), 2.05-1.95 (m, 2H), 1.89 (t, J = 6.6 Hz, 2H), 1.75-1.65 (m, 2H), 1.65-1.40 (m, 5H), 0.95 (d, J = 6.3 Hz, 6H). |
| 126 | 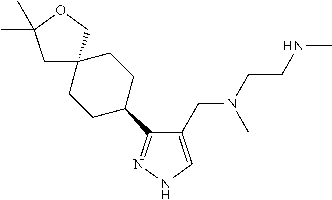 | LCMS: 2.11 min, m/z = 335 [M + 1]. ¹H NMR (500 MHz, CDCl₃) δ: 7.37 (s, 1H), 3.75 (s, 2H), 3.35 (s, 2H), 2.73-2.62 (m, 3H), 2.50 (t, J = 6.0 Hz, 2H), 2.43 (s, 3H), 2.14 (s, 3H), 1.93-1.80 (m, 4H), 1.62 (s, 2H), 1.58-1.43 (m, 4H), 1.27 (s, 6H). |
| 47 | 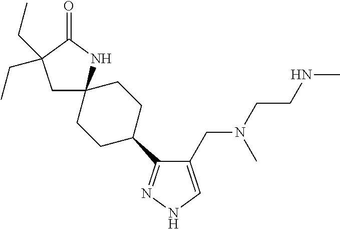 [SMILES: CCC1(CC)C[C@]2(CC[C@H](c3n[nH]cc3CN(C)CCNC)CC2)NC1=O] | LCMS: m/z = 376.84 [M + 1]. ¹H-NMR (400 MHz, D2O) δ: 7.70 (s, 1H), 4.27 (s, 2H), 3.42 (m, 4H), 2.72-2.67 (m, 7H), 1.81 (s, 2H), 1.76-1.53 (m, 8H), 1.39-1.36 (q, 4H), 0.73 (t, 6H). |
| 48 | 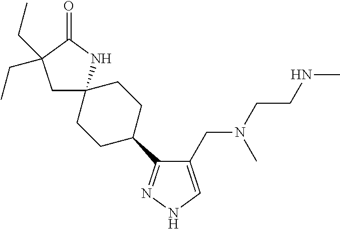 [SMILES: CCC1(CC)C[C@]2(CC[C@@H](c3n[nH]cc3CN(C)CCNC)CC2)NC1=O] | LCMS: m/z = 376.8 [M + 1]. ¹H-NMR (400 MHz, D2O) δ: 7.73 (s, 1H), 4.27 (s, 2H), 3.49 (m, 4H), 2.72-2.66 (m, 7H), 1.89 (s, 2H), 1.72-1.52 (m, 8H), 1.40-1.36 (q, 4H), 0.74 (t, 6H). |
| 22 | 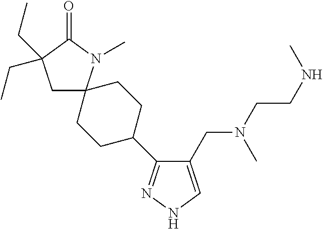 [SMILES: CCC1(CC)CC2(CCC(c3n[nH]cc3CN(C)CCNC)CC2)N(C)C1=O] | ¹H-NMR (400 MHz, D2O): 7.79 (s, 1H), 4.31 (s, 2H), 3.78-3.44 (m, 4H), 2.75 (s, 3H), 2.69 (m, 7H), 1.89 (s, 2H), 1.85-1.79 (m, 4H), 1.70-1.67 (t, 2H), 1.51-1.48 (t, 2H) 1.43-1.38 (q, 4H), 0.71-0.68 (t, 6H). |
| 23 | 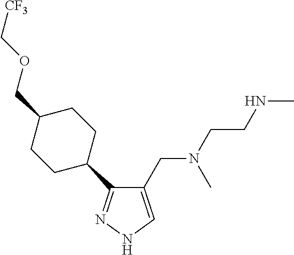 [SMILES: CNCCN(C)Cc1c[nH]nc1[C@H]1CC[C@@H](COCC(F)(F)F)CC1] | LCMS: m/z = 363.23 [M + 1]. ¹H NMR (400 MHz, D₂O) δ: 7.58 (s, 1H), 4.21 (s, 2H), 3.84-3.79 (q, J = 9.2 Hz, 2H), 3.38-3.33 (m, 6H), 2.64 (s, 3H), 2.58 (m, 4H), 1.70-1.67 (m, 4H), 1.54 (m, 3H), 1.40-1.30 (m, 2H). |
| 24 | 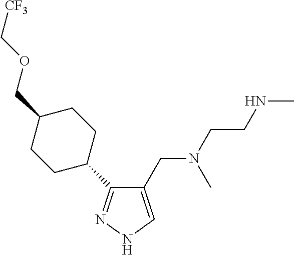 [SMILES: CNCCN(C)Cc1c[nH]nc1[C@H]1CC[C@H](COCC(F)(F)F)CC1] | LCMS: m/z = 363.23 [M + 1]. ¹H NMR (400 MHz, D₂O) δ: 7.69 (s, 1H), 4.27 (s, 2H), 3.96-3.89 (m, 2H), 3.71-3.69 (d, J = 7.6 Hz 2H), 3.42 (s, 4H), 2.73 (s, 4H), 2.67 (m, 3H), 1.96 (bs, 1H), 1.63-1.55 (m, 8H). |
| 16 | 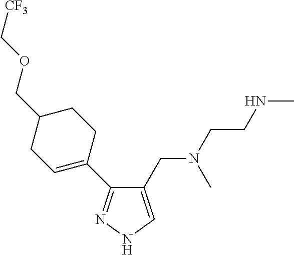 [SMILES: CNCCN(C)Cc1c[nH]nc1C1=CCC(COCC(F)(F)F)CC1] | LCMS: m/z = 361.44 [M + 1]. ¹H NMR (400 MHz, MeOH) δ: 7.82 (s, 1H), 6.05 (s, 1H), 4.43 (s, 2H), 3.98-3.92 (d, J = 9.2 Hz, 2H), 3.61-3.59 (d, J = 5.2 Hz, 2H), 3.51 (s, 4H), 2.84 (s, 3H), 2.79 (s, 3H), 2.48-2.39 (m, 3H), 2.03-2.01 (m, 3H), 1.56-1.46 (m, 1H). |
| 28 | 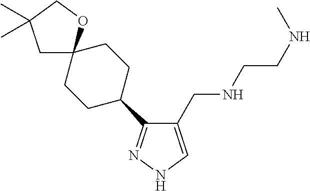 [SMILES: CNCCNCc1c[nH]nc1[C@H]1CC[C@@]2(CC1)CC(C)(C)CO2] | LCMS: m/z = 321.5 [M + 1]. ¹H NMR (400 MHz, D₂O) δ: 7.69 (s, 1H), 4.05 (s, 2H), 3.30 (s, 2H), 3.23 (bs, 4H), 2.69 (s, 1H), 2.55 (s, 3H), 1.76-1.73 (d, 2H), 1.53-1.37 (m, 8H), 0.84 (s, 6H). |
| 29 | 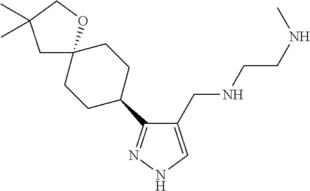 [SMILES: CNCCNCc1c[nH]nc1[C@H]1CC[C@]2(CC1)CC(C)(C)CO2] | LCMS: m/z = 321.48 [M + 1]. ¹H NMR (400 MHz, D₂O) δ: 7.54 (s, 1H), 3.93 (s, 2H), 3.22 (s, 4H), 3.12 (s, 2H), 2.55 (s, 1H), 2.44 (s, 3H), 1.58 (m, 4H), 1.45 (s, 2H), 1.23 (m, 4H), 0.76 (s, 6H). |
| 37 | 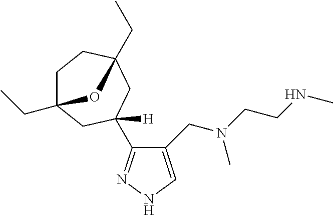 [SMILES: CC[C@]12CC[C@](CC)(C[C@H](c3n[nH]cc3CN(C)CCNC)C1)O2] | LCMS: m/z = 335.58 [M + 1]. ¹H-NMR (400 MHz, D₂O) δ: 7.67 (s, 1H), 4.24 (s, 2H), 3.42 (s, 4H), 3.22-3.18 (m, 1H), 2.71 (s, 3H), 2.68 (s, 3H), 1.87-1.85 (d, 2H), 1.74-1.68 (m, 4H), 1.54-1.49 (m, 6H), 0.79-0.75 (t, 6H). |
| 38 | 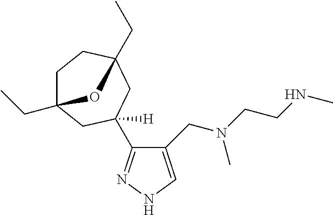 [SMILES: CC[C@]12CC[C@](CC)(C[C@@H](c3n[nH]cc3CN(C)CCNC)C1)O2] | LCMS: m/z = 335.58 [M + 1]. ¹H-NMR (400 MHz, D2O) δ: 7.67 (s, 1H), 4.21 (s, 2H), 3.40 (s, 4H), 2.93-2.89 (m, 1H), 2.72 (s, 3H), 2.65 (s, 3H), 1.96-1.91 (m, 2H), 1.71-1.45 (m, 10H), 0.78-0.73 (t, 6H). |
| 36 | 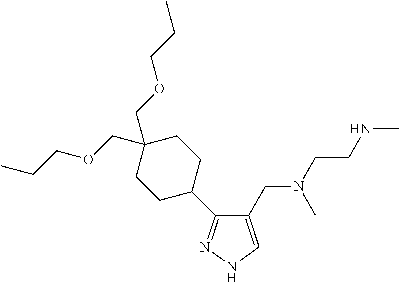 [SMILES: CCCOCC1(COCCC)CCC(c2n[nH]cc2CN(C)CCNC)CC1] | LCMS: m/z = 395.35 [M + 1]. ¹H NMR (400 MHz, D₂O) δ: 7.78 (s, 1H), 4.30 (s, 2H), 3.50-3.16 (m, 10H), 3.22 (s, 2H), 2.75 (s, 3H), 2.71-2.69 (m, 1H), 2.70 (s, 3H), 1.62-1.57 (m, 6H), 1.52-1.45 (m, 4H), 1.30-1.20 (m, 2H), 0.79 (t, J = 7.6 Hz, 6H). |
| 44 | 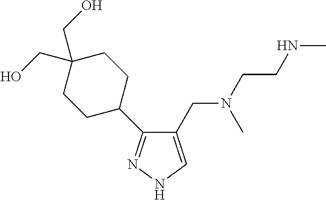 [SMILES: CNCCN(C)Cc1c[nH]nc1C1CCC(CO)(CO)CC1] | LCMS: m/z = 311.25 [M + 1]. ¹H NMR (400 MHz, MeOD) δ: 7.74 (s, 1H), 4.35 (s, 2H), 3.74 (s, 2H), 3.53 (s, 4H), 3.42 (s, 2H), 3.34- 3.32 (m, 1H), 2.87 (s, 3H), 2.79 (s, 3H), 1.81- 1.70 (m, 6H), 1.43-1.31 (m, 2H). |
| 39 | 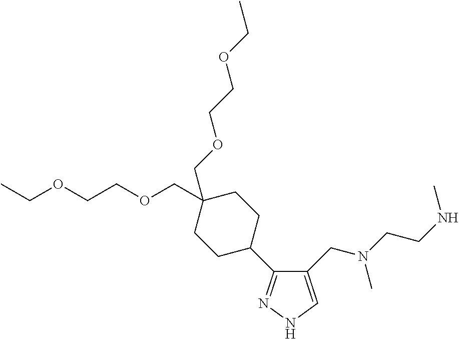 [SMILES: CCOCCOCC1(COCCOCC)CCC(c2n[nH]cc2CN(C)CCNC)CC1] | LCMS: m/z = 455.11 [M + 1]. ¹H NMR (400 MHz, D₂O) δ: 7.75 (s, 1H), 4.28 (s, 2H), 3.56-3.54 (m, 10H), 3.48-3.43 (m, 8H), 3.24 (s, 2H), 2.73 (s, 3H), 2.67 (s, 4H), 1.65-1.55 (m, 6H), 1.28-1.22 (m, 2H), 1.08-1.03 (m, 6H). |
| 41 | 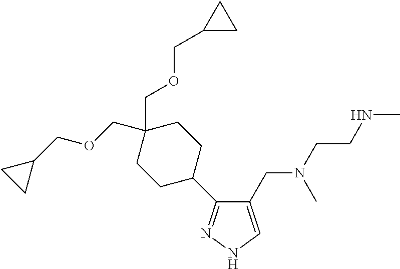 [SMILES: CNCCN(C)Cc1c[nH]nc1C1CCC(COCC2CC2)(COCC2CC2)CC1] | LCMS: m/z = 419.7 [M + 1]. ¹H-NMR (400 MHz, D₂O) δ: 7.70 (s, 1H), 4.27 (s, 2H), 3.55-3.43 (m, 6H), 3.26-3.22 (m, 6H), 2.73 (s, 3H), 2.68 (s, 3H), 1.64-1.56 (m, 5H), 1.25 (m, 2H), 0.95 (m, 2H), 0.44 (m, 4H), 0.10 (brs, 4H). |
| 42 | 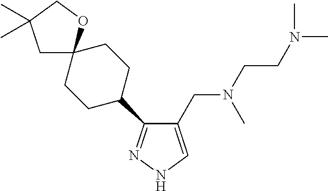 | LCMS: m/z = 349.6 [M + 1]. ¹H-NMR (400 MHz, D2O) δ: 7.77 (s, 1H), 4.30 (s, 2H), 3.55 (s, 4H), 3.43 (s, 2H), 3.22 (s, 3H), 2.86 (s, 6H), 2.59 (s, 4H), 1.93-1.86 (m, 2H), 1.65- 1.40 (m, 8H), 0.97 (s, 6H). |
| 43 | 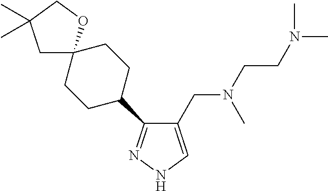 | LCMS: m/z = 349.6 [M + 1]. ¹H-NMR (400 MHz, D2O) δ: 7.76 (s, 1H), 4.26 (s, 2H), 3.52 (s, 4H), 3.45 (s, 2H), 2.84 (s, 6H), 2.71 (s, 4H), 1.83-1.76 (m, 4H), 1.69 (s, 2H), 1.50- 1.44 (m, 4H), 0.98 (s, 6H). |
| 51 | 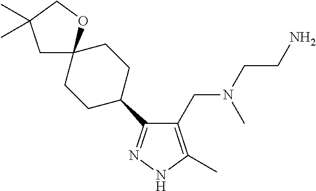 [SMILES: Cc1[nH]nc([C@H]2CC[C@@]3(CC2)CC(C)(C)CO3)c1CN(C)CCN] | LCMS: m/z = 336.0 [M + 1]. ¹H NMR (400 MHz, D₂O) δ: 4.25 (s, 2H), 3.35-3.45 (m, 6H), 2.70-2.74 (m, 4H), 2.23 (s, 3H), 1.84-1.87 (m, 2H), 1.43-1.51 (s, 8H), 0.95 (s, 6H). |
| 52 | 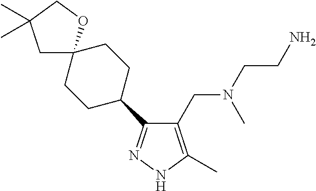 [SMILES: Cc1[nH]nc([C@H]2CC[C@]3(CC2)CC(C)(C)CO3)c1CN(C)CCN] | LCMS: m/z = 335.8 [M + 1]. ¹H NMR (400 MHz, D₂O) δ: 4.22 (s, 2H), 3.47 (s, 2H), 3.36- 3.45 (m, 4H), 2.74 (s, 3H), 2.58-2.63 (m, 1H), 2.38 (s, 3H), 1.84-1.73 (m, 4H), 1.68 (s, 2H), 1.39-1.52 (m, 4H), 0.99 (s, 6H). |
| 49 | 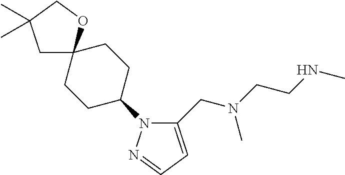 | LCMS: m/z = 335.7 [M + 1]. ¹H-NMR (400 MHz, D₂O) δ: 7.55-7.54 (d, J = 1.6 Hz, 1H), 6.48-6.47 (d, J = 2.0 Hz, 1H), 4.57-4.51 (d, 2H), 4.23-4.17 (m, 1H), 3.50-3.46 (m, 2H), 3.42-3.37 (m, 4H), 2.75 (s, 3H), 2.65 (s, 3H), 2.03-1.87 (m, 4H), 1.66-1.63 (m, 2H), 1.56- 1.09 (m, 4H), 1.07 (s, 6H). |
| 50 | 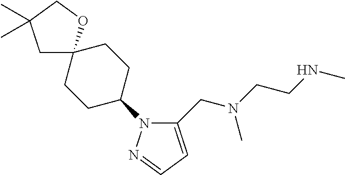 | LCMS: m/z = 335.7 [M + 1]. ¹H-NMR (400 MHz, D₂O) δ: 7.54 (d, J = 1.6 Hz, 1H), 6.47 (d, J = 2.0 Hz, 1H), 4.52 (s, 2H), 4.22-4.21 (m, 1H), 3.51-3.48 (m, 2H), 3.43-3.38 (m, 4H), 2.75 (s, 3H), 2.65 (s, 3H), 1.83-1.81 (m, 6H), 1.71 (s, 2H), 1.58-1.50 (m, 2H), 0.94 (s, 6H). |
| 46 | 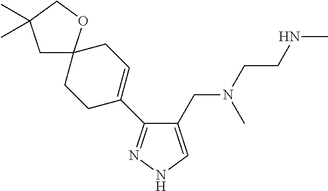 | LCMS: m/z = 333.6 [M + 1]. ¹H-NMR (400 MHz, D₂O) δ: 7.71 (s, 1H), 5.83 (s, 1H), 4.33-4.31 (m, 2H), 3.46 (s, 2H), 3.46 (s, 4H), 2.68 (s, 3H), 2.65 (s, 3H), 2.37-2.32 (m, 4H), 1.85-1.98 (m, 1H), 1.74-1.57 (m, 3H), 0.99 (s, 6H). |
| 15 | 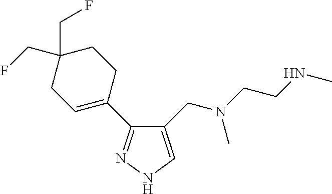 [SMILES: CNCCN(C)Cc1c[nH]nc1C1=CCC(CF)(CF)CC1] | ¹H NMR (400 MHz, D₂O) δ: 7.83 (s, 1H), 6.01 (s, 1H), 4.48-4.44 (q, 2H), 4.397 (s, 2H), 3.36-4.32 (m, 2H), 3.50-3.45 (m, 4H), 2.83 (s, 3H), 2.79 (s, 3H), 2.49 (bs, 2H), 2.21 (bs, 2H), 2.81-2.78 (t, 2H). |
| 33 | 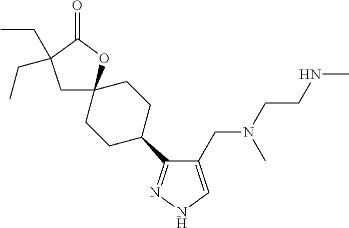 [SMILES: CCC1(CC)C[C@]2(CC[C@H](c3n[nH]cc3CN(C)CCNC)CC2)OC1=O] | LCMS: m/z = 377.49 [M + 1]. ¹H NMR (400 MHz, D₂O) δ: 7.59 (s, 1H), 4.15 (s, 2H), 3.36- 3.22 (m, 4H), 2.68 (m, 1H), 2.60 (s, 3H), 2.05 (s, 3H), 1.85-1.80 (m, 4H), 1.56-1.48 (m, 6H), 1.41-1.33 (m, 4H), 0.66-0.63 (t, 6H). |
| 34 | 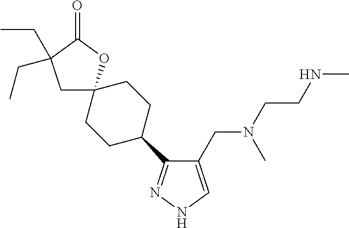 [SMILES: CCC1(CC)C[C@]2(CC[C@@H](c3n[nH]cc3CN(C)CCNC)CC2)OC1=O] | LCMS: m/z = 377.49 [M + 1]. ¹H NMR (400 MHz, D₂O) δ: 7.60 (s, 1H), 4.16 (s, 2H), 3.39- 3.26 (m, 4H), 2.61 (s, 4H), 2.55 (s, 3H), 1.99 (s, 2H), 1.78-1.61 (m, 6H), 1.56- 1.39 (m, 6H), 0.68-0.65 (t, 6H). |
| 40 | 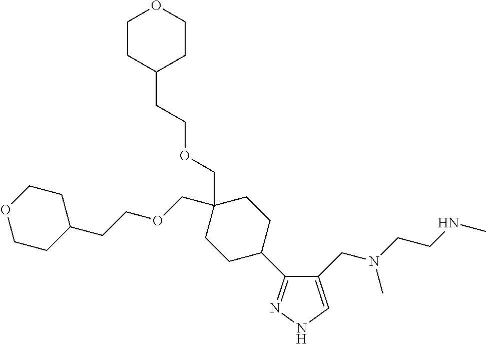 [SMILES: CNCCN(C)Cc1c[nH]nc1C1CCC(COCCC2CCOCC2)(COCCC2CCOCC2)CC1] | LCMS: m/z = 534.77 [M + 1]. ¹H NMR (400 MHz, D₂O) δ: 7.62 (s, 1H), 4.19 (s, 2H), 5.02- 5.00 (m, 4H), 3.45-3.36 (m, 8H), 3.31-3.33 (m, 4H), 3.12 (s, 2H), 2.65 (s, 3H), 2.60 (m, 4H), 1.55-1.48 (m, 10H), 1.39-1.36 (m, 4H), 1.14- 1.06 (m, 8H). |
| 45 | 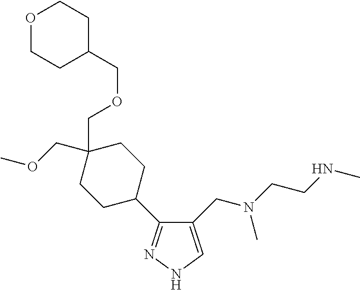 | ESI-MS: m/z = 423.75 [M + 1]. ¹H NMR (400 MHz, D₂O) δ: 7.63 (s, 1H), 4.19 (s, 2H), 3.86- 3.83 (m, 2H), 3.47-3.15 (m, 15H), 2.67-2.65 (m, 6H), 1.85-1.63 (s, 2H), 1.56 (m, 7H), 1.20- 1.14 (m, 5H). |
| 125 | 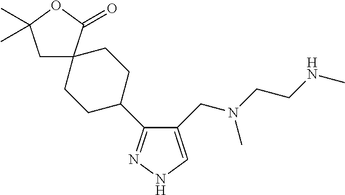 | LCMS: 1.49 and 1.51 min, m/z = 349 [M + 1]. ¹H NMR (250 MHz, MeOD) δ: 7.44 (d, J = 3.3 Hz, 1H), 3.44 (d, J = 4.4 Hz, 2H), 2.94-2.74 (m, 1H), 2.73-2.62 (m, 2H), 2.52 (t, J = 6.3 Hz, 2H), 2.36 (d, J = 5.4 Hz, 3H), 2.29-2.02 (m, 7H), 1.95-1.56 (m, 6H), 1.46 (d, J = 5.8 Hz, 6H). |
| 57 | 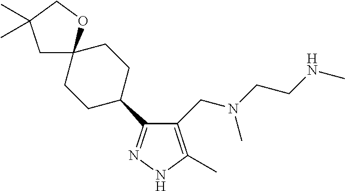 | LCMS: 1.59 min, m/z = 349.7 [M + 1]. ¹H NMR (400 MHz, D₂O) δ: 4.21 (brs, 2H), 3.43 (m, 6H), 2.73 (s, 3H), 2.70- 2.59 (m, 1H), 2.68 (s, 3H), 2.17 (s, 3H), 1.86 (d, J = 13 Hz, 2H), 1.65-1.54 (M, 4H), 1.55 (s, 2H), 1.50-1.41 (m, 2H), 0.96 (s, 6H). |
| 60 | 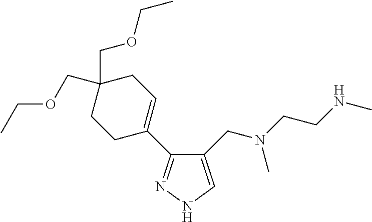 [SMILES: CCOCC1(COCC)CC=C(c2n[nH]cc2CN(C)CCNC)CC1] | LCMS: 1.62 min, m/z = 365.0 [M + 1]. ¹H NMR (400 MHz, D₂O) δ: 7.70 (s, 1H), 5.90 (s, 1H), 4.23 (s, 2H), 3.47 (q, J = 6 Hz, 4H), 3.40-3.19 (m, 8H), 2.65 (s, 3H), 2.63 (s, 3H), 2.26 (brs, 2H), 1.99 (s, 2H), 1.56 (m, 2H), 1.07 (t, J = 6 Hz, 6H). |
| 61 | 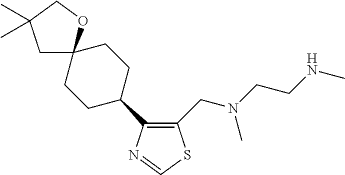 | LCMS: 1.81 min, m/z = 352.8 [M + 1]. ¹H NMR (400 MHz, D₂O) δ: 8.80 (s, 1H), 4.37 (s, 2H), 3.42 (s, 6H), 2.94 (brt, J = 9 Hz, 1H), 2.73 (s, 3H), 2.66 (s, 3H), 1.86 (d, J = 12 Hz, 2H), 1.75-1.65 (m, 2H), 1.62-1.43 (m, 6H), 0.96 (s 6H). |
| 123 | 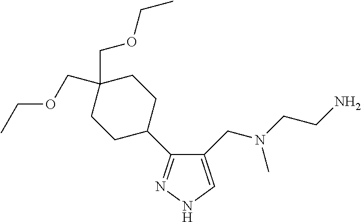 [SMILES: CCOCC1(COCC)CCC(c2n[nH]cc2CN(C)CCN)CC1] | LCMS: 1.50 min, m/z = 353.5 [M + 1]. ¹H NMR (400 MHz, D₂O) δ: 7.76 (s, 1H), 4.28 (s, 2H), 3.54-3.33 (m, 10H), 3.19 (s, 2H), 2.73 (s, 3H), 2.71-2.63 (m, 1H), 1.65-1.48 (m, 6H), 1.30-1.17 (m, 2H), 1.08 (q, J = 7 Hz, 6H). |

### Biological Methods

### PRMT1 Biochemical Assay

General Materials.

S-adenosylmethionine (SAM), S-adenosylhomocysteine (SAH), bicine, Tween20, dimethylsulfoxide (DMSO), bovine skin gelatin (BSG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 36-50 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-RLARRGGVKRISGLI-amide (SEQ ID NO.:1).

Molecular Biology:

Full-length human PRMT1 isoform 1 (NM_001536.5) transcript clone was amplified from an HEK 293 cDNA library, incorporating flanking 5′ sequence encoding a FLAG tag (DYKDDDDK) (SEQ ID NO.:2) fused directly to Met 1 of PRMT1. The amplified gene was subcloned into pFastBacI (Life Technologies) modified to encode an N-terminal GST tag and a TEV cleavage sequence

(SEQ ID NO.: 3)
(MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGAV
LDIRYGVSRIAYSKDFETLKVDFLSKLPEMLKMFEDRLCHKTYLNGDHVT
HPDFMLYDALDVVLYMDPMCLDAFPKLVCFKKRIEAIPQIDKYLKSSKYI
AWPLQGWQATFGGGDHPPKSDENLYFQGGNS) fused to Asp of
the Flag tag of PRMT1.

Protein Expression.

Recombinant baculovirus were generated according to Bac-to-Bac kit instructions (Life Technologies). Protein over-expression was accomplished by infecting exponentially growing High Five insect cell culture at 1.5×10⁶ cell/ml with 1:100 ratio of virus. Infections were carried out at 27° C. for 48 hours, harvested by centrifugation, and stored at −80° C. for purification.

Protein Purification.

Expressed full-length human GST-tagged PRMT1 protein was purified from cell paste by glutathione sepharose affinity chromatography after equilibration of the resin with 50 mM phosphate buffer, 200 mM NaCl, 5% glycerol, 5 mM β-mercaptoethanol, pH7.8 (Buffer A). GST-tagged PRMT1 was eluted with 50 mM Tris, 2 mM glutathione, pH 7.8, dialysed in buffer A and concentrated to 1 mg/mL. The purity of recovered protein was 73%. Reference: Wasilko, D. J. and S. E. Lee: “TIPS: titerless infected-cells preservation and scale-up” Bioprocess J., 5 (2006), pp. 29-32.

Predicted Translations:

GST-tagged PRMT1
(SEQ ID NO.: 4)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDENLYFQGGNSDYKDDDDKMAAAEAANCIME
NFVATLANGMSLQPPLEEVSCGQAESSEKPNAEDMTSKDYYFDSYAHFGI
HEEMLKDEVRTLTYRNSMFHNRHLFKDKVVLDVGSGTGILCMFAAKAGAR
KVIGIECSSISDYAVKIVKANKLDHVVTIIKGKVEEVELPVEKVDIIISE
WMGYCLFYESMLNTVLYARDKWLAPDGLIFPDRATLYVTAIEDRQYKDYK
IHWWENVYGFDMSCIKDVAIKEPLVDVVDPKQLVTNACLIKEVDIYTVKV
EDLTFTSPFCLQVKRNDYVHALVAYFNIEFTRCHKRTGFSTSPESPYTHW
KQTVFYMEDYLTVKTGEEIFGTIGMRPNAKNNRDLDFTIDLDFKGQLCEL
SCSTDYRMR

General Procedure for PRMT1 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT1, was added to columns 11, 12, 23, 24, rows I-P for the minimum signal control. A cocktail (40 ul) containing the PRMT1 enzyme was added by Multidrop Combi (Thermo-Fisher). The compounds were allowed to incubate with PRMT1 for 30 min at room temperature, then a cocktail (10 ul) containing SAM and peptide was added to initiate the reaction (final volume=51 ul). The final concentrations of the components were as follows: PRMT1 was 0.5 nM, ³H-SAM was 200 nM, non-radiolabeled SAM was 1.5 uM, peptide was 20 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 \vert {( \begin{matrix}X \\ {IC}_{50}\end{matrix} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT6 Biochemical Assay

General Materials.

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 36-50 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-RLARRGGVKRISGLI-amide and contained a monomethylated lysine at position 44 (SEQ ID NO.:5).

Molecular Biology:

Full-length human PRMT6 (NM_018137.2) transcript clone was amplified from an HEK 293 cDNA library, incorporating a flanking 5′ sequence encoding a FLAG tag (MDYKDDDDK) (SEQ ID NO.:6) fused directly to Ser 2 of PRMT6 and a 3′ sequence encoding a hexa His sequence (HHHHHH) (SEQ ID NO.: 17) fused directly to Asp 375. The amplified gene was subcloned into pFastBacMam (Viva Biotech).

Protein Expression.

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

Protein Purification.

Expressed full-length human Flag- and His-tagged PRMT6 protein was purified from cell paste by NiNTA agarose affinity chromatography after equilibration of the resin with buffer containing 50 mM Tris, 300 mM NaCl, 10% glycerol, pH 7.8 (Buffer Ni-A). Column was washed with 20 mM imidazole in the same buffer and Flag-PRMT6-His was eluted with 150 mM imidazole. Pooled fractions were dialysed against buffer Ni-A and further purified by anti-flag M2 affinity chromatography. Flag-PRMT6-His was eluted with 200 ug/ml FLAG peptide in the same buffer. Pooled fractions were dialysed in 20 mM Tris, 150 mM NaCl, 10% glycerol and 5 mM β-mercaptoethanol, pH 7.8. The purity of recovered protein was 95%.

Predicted Translations:

Flag-PRMT6-His
(SEQ ID NO.: 7)
MDYKDDDDKSQPKKRKLESGGGGEGGEGTEEEDGAEREAALERPRRTKRE
RDQLYYECYSDVSVHEEMIADRVRTDAYRLGILRNWAALRGKTVLDVGAG
TGILSIFCAQAGARRVYAVEASAIWQQAREVVRFNGLEDRVHVLPGPVET
VELPEQVDAIVSEWMGYGLLHESMLSSVLHARTKWLKEGGLLLPASAELF
IAPISDQMLEWRLGFWSQVKQHYGVDMSCLEGFATRCLMGHSEIVVQGLS
GEDVLARPQRFAQLELSRAGLEQELEAGVGGRFRCSCYGSAPMHGFAIWF
QVTFPGGESEKPLVLSTSPFHPATHWKQALLYLNEPVQVEQDTDVSGEIT
LLPSRDNPRRLRVLLRYKVGDQEEKTKDFAMEDHHHHHH

General Procedure for PRMT6 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT6, was added to columns 11, 12, 23, 24, rows I-P for the minimum signal control. A cocktail (40 ul) containing the PRMT6 enzyme was added by Multidrop Combi (Thermo-Fisher). The compounds were allowed to incubate with PRMT6 for 30 min at room temperature, then a cocktail (10 ul) containing SAM and peptide was added to initiate the reaction (final volume=51 ul). The final concentrations of the components were as follows: PRMT6 was 1 nM, ³H-SAM was 200 nM, non-radiolabeled SAM was 250 nM, peptide was 75 nM, SAH in the minimum signal control wells was 1 mM, and the DMSO concentration was 2%. The assays were stopped by the addition of non-radiolabeled SAM (10 ul) to a final concentration of 400 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 \vert {( \begin{matrix}X \\ {IC}_{50}\end{matrix} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT8 Biochemical Assay

General Materials.

S-adenosylmethionine (SAM), S-adenosylhomocysteine (SAH), bicine, Tween20, dimethylsulfoxide (DMSO), bovine skin gelatin (BSG), isopropyl-β-D-thiogalactopyranoside (IPTG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 31-45 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-KPAIRRLARRGGVKR-amide (SEQ ID NO.:8).

Molecular Biology:

Full-length human PRMT8 (NM_019854.4) isoform 1 transcript clone was amplified from an HEK 293 cDNA library and subcloned into pGEX-4T-1 (GE Life Sciences). The resulting construct encodes an N-terminal GST tag and a thrombin cleavage sequence

(SEQ ID NO.: 9)
(MSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEG
AVLDIRYGVSRIAYSKDFETLKVDFLSKLPEMLKMFEDRLCHKTYLNGD
HVTHPDFMLYDALDVVLYMDPMCLDAFPKLVCFKKRIEAIPQIDKYLKS
SKYIAWPLQGWQATFGGGDHPPKSDLVPRGSPEF) fused
directly to Met 1 of PRMT8.

Protein Expression.

E. coli (BL21(DE3) Gold, Stratagene) made competent by the CaCl₂ method were transformed with the PRMT8 construct and ampicillin selection. Protein over-expression was accomplished by growing the PRMT8 expressing E. coli clone and inducing expression with 0.3 mM IPTG at 16° C. The culture was grown for 12 hours, harvested by centrifugation, and stored at −80° C. for purification.

Protein Purification.

Expressed full-length human GST-tagged PRMT8 protein was purified from cell paste by glutathione sepharose affinity chromatography after the resin was equilibrated with 50 mM phosphate buffer, 200 mM NaCl, 5% glycerol, 5 mM β-mercaptoethanol, pH7.8 (Buffer A). GST-tagged PRMT8 was eluted with 50 mM Tris, 2 mM glutathione, pH 7.8. Pooled fractions were cleaved by thrombin (10U) and dialysed in buffer A. GST was removed by reloading the cleaved protein sample onto glutathione sepharose column and PRMT8 was collected in the flow-through fractions. PRMT8 was purified further by ceramic hydroxyapatite chromatography. The column was washed with 50 mM phosphate buffer, 100 mM NaCl, 5% glycerol, 5 mM j-mercaptoethanol, pH 7.8 and PRMT8 was eluted by 100 mM phosphate in the same buffer. Protein was concentrated and buffer was exchanged to 50 mM Tris, 300 mM NaCl, 10% glycerol, 5 mM β-mercaptoethanol, pH 7.8 by ultrafiltration. The purity of recovered protein was 89%.

Predicted Translations:

GST-tagged PRMT8
(SEQ ID NO.: 10)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSPEFMGMKHSSRCLLLRRKMAENAA
ESTEVNSPPSQPPQPVVPAKPVQCVHHVSTQPSCPGRGKMSKLLNPEEMT
SRDYYFDSYAHFGIHEEMLKDEVRTLTYRNSMYHNKHVFKDKVVLDVGSG
TGILSMFAAKAGAKKVFGIECSSISDYSEKIIKANHLDNIITIFKGKVEE
VELPVEKVDIIISEWMGYCLFYESMLNTVIFARDKWLKPGGLMFPDRAAL
YVVAIEDRQYKDFKIHWWENVYGFDMTCIRDVAMKEPLVDIVDPKQVVTN
ACLIKEVDIYTVKTEELSFTSAFCLQIQRNDYVHALVTYFNIEFTKCHKK
MGFSTAPDAPYTHWKQTVFYLEDYLTVRRGEEIYGTISMKPNAKNVRDLD
FTVDLDFKGQLCETSVSNDYKMR

General Procedure for PRMT8 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT8, was added to columns 11, 12, 23, 24, rows I-P for the minimum signal control. A cocktail (40 ul) containing the PRMT8 enzyme was added by Multidrop Combi (Thermo-Fisher). The compounds were allowed to incubate with PRMT8 for 30 min at room temperature, then a cocktail (10 ul) containing ³H-SAM and peptide was added to initiate the reaction (final volume=51 ul). The final concentrations of the components were as follows: PRMT8 was 1.5 nM, ³H-SAM was 50 nM, non-radiolabeled SAM was 550 nM, peptide was 150 nM, SAH in the minimum signal control wells was 1 mM, and the DMSO concentration was 2%. The assays were stopped by the addition of non-radiolabeled SAM (10 ul) to a final concentration of 400 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 \vert {( \begin{matrix}X \\ {IC}_{50}\end{matrix} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT3 Biochemical Assay

General Materials.

S-adenosylmethionine (SAM), S-adenosylhomocysteine (SAH), bicine, Tween20, dimethylsulfoxide (DMSO), bovine skin gelatin (BSG), isopropyl-β-D-thiogalactopyranoside (IPTG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide containing the classic RMT substrate motif was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-GGRGGFGGRGGFGGRGGFG-amide (SEQ ID NO.:11).

Molecular Biology:

Full-length human PRMT3 (NM_005788.3) isoform 1 transcript clone was amplified from an HEK 293 cDNA library and subcloned into pGEX-KG (GE Life Sciences). The resulting construct encodes an N-terminal GST tag and a thrombin cleavage sequence

(SEQ ID NO.: 12)
(MSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEG
AVLDIRYGVSRIAYSKDFETLKVDFLSKLPEMLKMFEDRLCHKTYLNGD
HVTHPDFMLYDALDVVLYMDPMCLDAFPKLVCFKKRIEAIPQIDKYLKS
SKYIAWPLQGWQATFGGGDHPPKSDLVPRGS)fused directly to
Cys 2 of PRMT3.

Protein Expression.

E. coli (BL21(DE3) Gold, Stratagene) made competent by the CaCl₂ method were transformed with the PRMT3 construct and ampicillin selection. Protein over-expression was accomplished by growing the PRMT3 expressing E. coli clone and inducing expression with 0.3 mM IPTG at 16° C. The culture was grown for 12 hours, harvested by centrifugation, and stored at −80° C. for purification.

Protein Purification.

Expressed full-length human GST-tagged PRMT3 protein was purified from cell paste by glutathione sepharose affinity chromatography after equilibration of the resin with 50 mM phosphate buffer, 200 mM NaCl, 5% glycerol, 1 mM EDTA, 5 mM β-mercaptoethanol, pH6.5 (Buffer A). GST-tagged PRMT3 was eluted with 50 mM Tris, 2 mM glutathione, pH 7.1 and 50 mM Tris, 20 mM glutathione, pH 7.1. Pooled fractions were dialysed in 20 mM Tris, 50 mM NaCl, 5% glycerol, 1 mM EDTA, 1 mM DTT, pH7.5 (Buffer B) and applied to a Q Sepharose Fast Flow column. GST-tagged PRMT3 was eluted by 500 mM NaCl in buffer B. Pooled fractions were dialyzed in 25 mM phosphate buffer, 100 mM NaCl, 5% glycerol, 2 mM DTT, pH 6.8 (Buffer C) and loaded on to a ceramic hydroxyapatite column. GST-tagged PRMT3 eluted with 25-400 mM phosphate in buffer C. Protein was concentrated and buffer was exchanged to 20 mM Tris, 150 mM NaCl, 5% glycerol, 5 mM β-mercaptoethanol, pH7.8 by ultrafiltration. The purity of recovered protein was 70%.

Predicted Translations:

GST-tagged PRMT3
(SEQ ID NO.: 13)
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRGSCSLASGATGGRGAVENEED
LPELSDSGDEAAWEDEDDADLPHGKQQTPCLFCNRLFTSAEETFSHCKS
EHQFNIDSMVHKHGLEFYGYIKLINFIRLKNPTVEYMNSIYNPVPWEKE
EYLKPVLEDDLLLQFDVEDLYEPVSVPFSYPNGLSENTSVVEKLKHMEA
RALSAEAALARAREDLQKMKQFAQDFVMHTDVRTCSSSTSVIADLQEDE
DGVYFSSYGHYGIHEEMLKDKIRTESYRDFIYQNPHIFKDKVVLDVGCG
TGILSMFAAKAGAKKVLGVDQSEILYQAMDIIRLNKLEDTITLIKGKIE
EVHLPVEKVDVIISEWMGYFLLFESMLDSVLYAKNKYLAKGGSVYPDIC
TISLVAVSDVNKHADRIAFWDDVYGFKMSCMKKAVIPEAVVEVLDPKTL
ISEPCGIKHIDCHTTSISDLEFSSDFTLKITRTSMCTAIAGYFDIYFEK
NCHNRVVFSTGPQSTKTHWKQTVFLLEKPFSVKAGEALKGKVTVHKNKK
DPRSLTVTLTLNNSTQTYGLQ

General Procedure for PRMT3 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT3, was added to columns 11, 12, 23, 24, rows I-P for the minimum signal control. A cocktail (40 ul) containing the PRMT3 enzyme was added by Multidrop Combi (Thermo-Fisher). The compounds were allowed to incubate with PRMT3 for 30 min at room temperature, then a cocktail (10 ul) containing SAM and peptide was added to initiate the reaction (final volume=51 ul). The final concentrations of the components were as follows: PRMT3 was 0.5 nM, ³H-SAM was 100 nM, non-radiolabeled SAM was 1.8 uM, peptide was 330 nM, SAH in the minimum signal control wells was 1 mM, and the DMSO concentration was 2%. The assays were stopped by the addition of potassium chloride (10 ul) to a final concentration of 100 mM. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 \vert {( \begin{matrix}X \\ {IC}_{50}\end{matrix} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### CARM1 Biochemical Assay

General Materials.

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H3 residues 16-30 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-PRKQLATKAARKSAP-amide and contained a monomethylated arginine at position 26 (SEQ ID NO.: 14).

Molecular Biology:

Human CARM1 (PRMT4) (NM_199141.1) transcript clone was amplified from an HEK 293 cDNA library, incorporating a flanking 5′ sequence encoding a FLAG tag (MDYKDDDDK) (SEQ ID NO.:6) fused directly to Ala 2 of CARM1 and 3′ sequence encoding a hexa His sequence (EGHHHHHH) (SEQ ID NO.: 15) fused directly to Ser 608. The gene sequence encoding isoform1 containing a deletion of amino acids 539-561 was amplified subsequently and subcloned into pFastBacMam (Viva Biotech).

Protein Expression.

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

Protein Purification.

Expressed full-length human Flag- and His-tagged CARM1 protein was purified from cell paste by anti-flag M2 affinity chromatography with resin equilibrated with buffer containing 20 mM Tris, 150 mM NaCl, 5% glycerol, pH 7.8. Column was washed with 500 mM NaCl in buffer A and Flag-CARM1-His was eluted with 200 ug/ml FLAG peptide in buffer A. Pooled fractions were dialyzed in 20 mM Tris, 150 mM NaCl, 5% glycerol and 1 mM DTT, pH 7.8. The purity of recovered protein was 94.

Predicted Translations:

Flag-CARM1-His
(SEQ ID NO.: 16)
MDYKDDDDKAAAAAAVGPGAGGAGSAVPGGAGPCATVSVFPGARLLTIG
DANGEIQRHAEQQALRLEVRAGPDSAGIALYSHEDVCVFKCSVSRETEC
SRVGKQSFIITLGCNSVLIQFATPNDFCSFYNILKTCRGHTLERSVFSE
RTEESSAVQYFQFYGYLSQQQNMMQDYVRTGTYQRAILQNHTDFKDKIV
LDVGCGSGILSFFAAQAGARKIYAVEASTMAQHAEVLVKSNNLTDRIVV
IPGKVEEVSLPEQVDIIISEPMGYMLFNERMLESYLHAKKYLKPSGNMF
PTIGDVHLAPFTDEQLYMEQFTKANFWYQPSFHGVDLSALRGAAVDEYF
RQPVVDTFDIRILMAKSVKYTVNFLEAKEGDLHRIEIPFKFHMLHSGLV
HGLAFWFDVAFIGSIMTVWLSTAPTEPLTHWYQVRCLFQSPLFAKAGDT
LSGTCLLIANKRQSYDISIVAQVDQTGSKSSNLLDLKNPFFRYTGTTPS
PPPGSHYTSPSENMWNTGSTYNLSSGMAVAGMPTAYDLSSVIASGSSVG
HNNLIPLGSSGAQGSGGGSTSAHYAVNSQFTMGGPAISMASPMSIPTNT
MHYGSEGHHHHHH

General Procedure for CARM1 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of CARM1, was added to columns 11, 12, 23, 24, rows I-P for the minimum signal control. A cocktail (40 ul) containing the CARM1 enzyme was added by Multidrop Combi (Thermo-Fisher). The compounds were allowed to incubate with CARM1 for 30 min at room temperature, then a cocktail (10 ul) containing ³H-SAM and peptide was added to initiate the reaction (final volume=51 ul). The final concentrations of the components were as follows: CARM1 was 0.25 nM, ³H-SAM was 30 nM, peptide was 250 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 \vert {( \begin{matrix}X \\ {IC}_{50}\end{matrix} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### RKO Methylation Assay

RKO adherent cells were purchased from ATCC (American Type Culture Collection), Manassas, Va., USA. DMEM/Glutamax medium, penicillin-streptomycin, heat inactivated fetal bovine serum, 0.05% trypsin and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Mono-methyl arginine antibody was purchased from Cell Signaling Technology, Danvers, Mass., USA. Methanol was purchased from VWR, Franklin, Mass., USA. 10% Tween 20 was purchased from KPL, Inc., Gaithersburg, Md., USA. DRAQ5 was purchased from Biostatus Limited, Leicestershire, UK.

RKO adherent cells were maintained in growth medium (DMEM/Glutamax medium supplemented with 10% v/v heat inactivated fetal bovine serum and 100 units/mL penicillin-streptomycin) and cultured at 37° C. under 5% CO₂.

Cell Treatment, in Cell Western (ICW) for Detection of Mono-Methyl Arginine and DNA Content.

RKO cells were seeded in assay medium at a concentration of 20,000 cells per mL to a poly-D-lysine coated 384 well culture plate (BD Biosciences 356697) with 50 μL per well. Compound (100 nL) from a 96-well source plate was added directly to 384 well cell plate. Plates were incubated at 37° C., 5% CO₂ for 72 hours. After three days of incubation, plates were brought to room temperature outside of the incubator for ten minutes and blotted on paper towels to remove cell media. 50 μL of ice cold 100% methanol was added directly to each well and incubated for 30 min at room temperature. After 30 min, plates were transferred to a Biotek EL406 plate washer and washed 2 times with 100 μL per well of wash buffer (1×PBS). Next 60 μL per well of Odyssey blocking buffer (Odyssey Buffer with 0.1% Tween 20 (v/v)) were added to each plate and incubated 1 hour at room temperature. Blocking buffer was removed and 20 μL per well of primary antibody was added (mono-methyl arginine diluted 1:200 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:200 800CW goat anti-rabbit IgG (H+L) antibody, 1:1000 DRAQ5 (Biostatus limited) in Odyssey buffer with 0.1% Tween 20 (v/v)) and incubated for 1 hour at room temperature. The plates were washed 5 times with 100 μL per well wash buffer then 2 times with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{monomethyl Arginine 800 nm value}{DRAQ 5 \; 700 nm value} )$$

Each plate included fourteen control wells of DMSO only treatment (minimum activation) as well as fourteen control wells for maximum activation treated with 20 μM of a reference compound. The average of the ratio values for each control type was calculated and used to determine the percent activation for each test well in the plate. Reference compound was serially diluted three-fold in DMSO for a total of nine test concentrations, beginning at 20 μM. Percent activation was determined and EC₃₀ curves were generated using triplicate wells per concentration of compound.

$$perrcent Activation = 100 - ( ( \frac{\begin{matrix}( Individual Test Sample Ratio ) - \\ ( Minimum Activation Ratio )\end{matrix}}{\begin{matrix}( Maximum Activation Ratio ) - \\ ( Minimum Activation Ratio )\end{matrix}} ) * 100 )$$

The biological activities of the exemplified compounds are shown in Table 2.

### OTHER EMBODIMENTS

The foregoing has been a description of certain non-limiting embodiments of the invention. Those of ordinary skill in the art will appreciate that various changes and modifications to this description may be made without departing from the spirit or scope of the present invention, as defined in the following claims.

What is claimed is:

1. A compound of Formula (S-I): 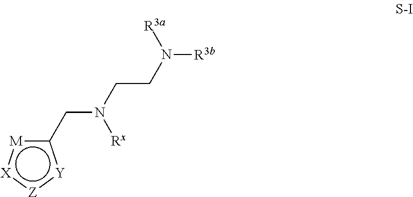
or a pharmaceutically acceptable salt thereof,
wherein
each of X, Y, and Z is independently O, S, N, NR⁴, or CR⁵, as valency permits;
R^{x} is optionally substituted C₁₋₄ alkyl or optionally substituted C₃₋₄ cycloalkyl;
M is —NR^{W1}— or —CR^{W2}—;
each of R^{W1} and R^{W2} is independently substituted cyclohexenyl, substituted cyclohexyl, or substituted tetrahydropyran;
each of R^{3a} and R^{3b} is independently hydrogen, optionally substituted C₁₋₄ alkyl, or optionally substituted C₃₋₄ cycloalkyl;
each instance of R⁴ is independently hydrogen or optionally substituted C₁₋₆ alkyl; and
each instance of R⁵ is independently hydrogen, halo, —CN, NO₂, optionally substituted C₁₋₄ alkyl, or optionally substituted C₃₋₄ cycloalkyl; and
provided that when M is —CR^{W2}—, at most one of X, Y, and Z is CR⁵;
provided that the compound is not one of the compounds in Table 1.

2. The compound of claim 1, wherein the compound is of Formula (S-I-a): 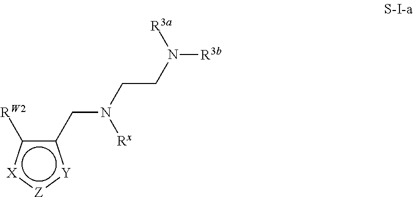
or a pharmaceutically acceptable salt thereof.

3. The compound of claim 1, wherein the compound is of Formula (S-II): 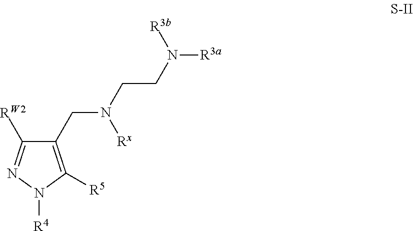
or a pharmaceutically acceptable salt thereof.

4. The compound of claim 1, wherein the compound is of Formula (S-III): 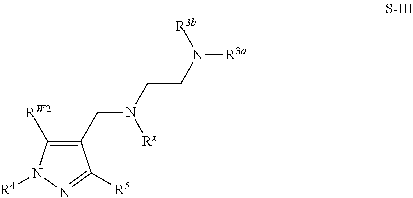
or a pharmaceutically acceptable salt thereof.

5. The compound of claim 1, wherein the compound is of Formula (S-IV): 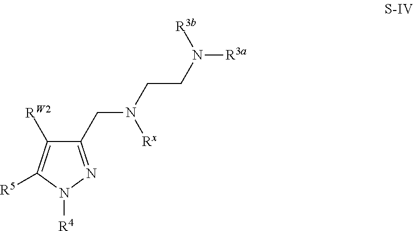
or a pharmaceutically acceptable salt thereof.

6. The compound of claim 1, wherein the compound is of Formula (S-V): 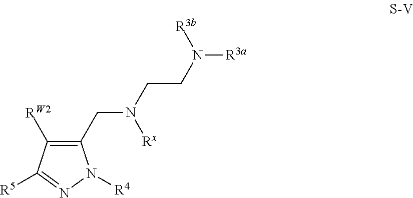
or a pharmaceutically acceptable salt thereof.

7. The compound of claim 1, wherein the compound is of Formula (S-VI): 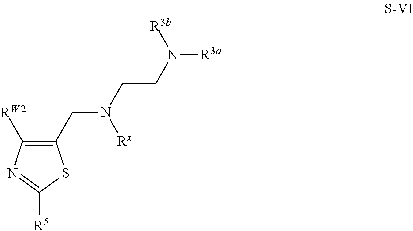
or a pharmaceutically acceptable salt thereof.

8. The compound of claim 1, wherein the compound is of Formula (S-VII): 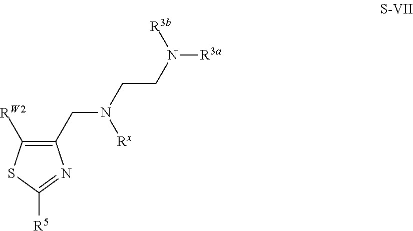
or a pharmaceutically acceptable salt thereof.

9. The compound of claim 1, wherein the compound is of Formula (S-VIII): 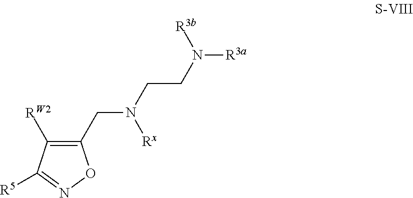
or a pharmaceutically acceptable salt thereof.

10. The compound of claim 1, wherein the compound is of Formula (S-IX): 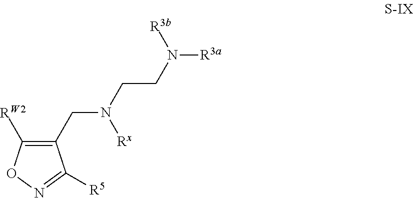
or a pharmaceutically acceptable salt thereof.

11. The compound of claim 1, wherein the compound is of Formula (S-X): 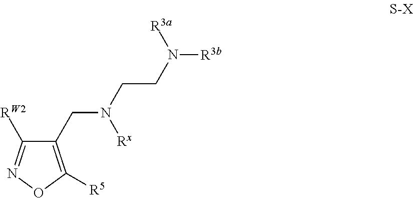
or a pharmaceutically acceptable salt thereof.

12. The compound of claim 1, wherein the compound is of Formula (S-XI): 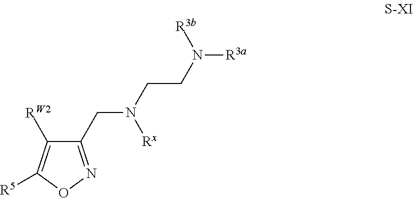
or a pharmaceutically acceptable salt thereof.

13. The compound of claim 1, wherein the compound is of Formula (S-XII): 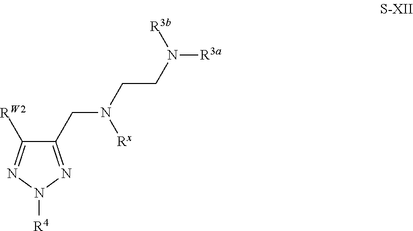
or a pharmaceutically acceptable salt thereof.

14. The compound of claim 1, wherein the compound is of Formula (S-XIII): 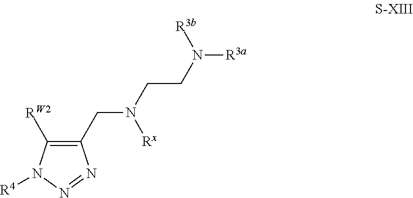
or a pharmaceutically acceptable salt thereof.

15. The compound of claim 1, wherein the compound is of Formula (S-XIV): 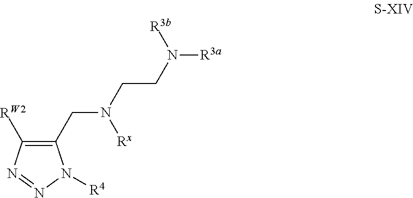
or a pharmaceutically acceptable salt thereof.

16. The compound of any one of claims 1-15, wherein R^{W2} is substituted cyclohexyl.

17. The compound of any one of claims 1-16, wherein R^{W2} is of Formula (S-i): 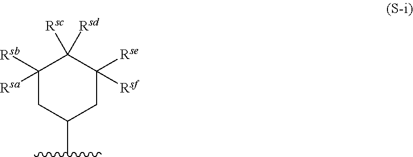
wherein:
each of R^{sa}, R^{sb}, R^{se}, and R^{sf} is independently hydrogen, optionally substituted C₁₋₆ alkyl, —OR^{SO}, or —C(═O)N(R^{SN1})₂;
each of R^{sc} and R^{sd} is independently optionally substituted C₁₋₆ alkyl, —OR^{SO}, —C(═O)N(R^{SN1})₂, or —N(R^{SN2})₂;
each instance of R^{SN1} and R^{SN2} is independently hydrogen or optionally substituted alkyl;
each instance of R^{SO} is optionally substituted alkyl, optionally substituted alkenyl, optionally substituted alkynyl, optionally substituted heterocyclyl, optionally substituted carbocyclyl, optionally substituted aryl, optionally substituted heteroaryl; and
R^{sa} and R^{sb} are optionally taken together with the intervening atom to form an optionally substituted carbocylic ring or an optionally substituted heterocyclic ring;
R^{sc} and R^{sd} are optionally taken together with the intervening atom to form an optionally substituted carbocylic ring or an optionally substituted heterocyclic ring; and
R^{se} and R^{sf} are optionally taken together with the intervening atom to form an optionally substituted carbocylic ring or an optionally substituted heterocyclic ring.

18. The compound of claim 17, wherein R^{W2} is of Formula (S-i-a):

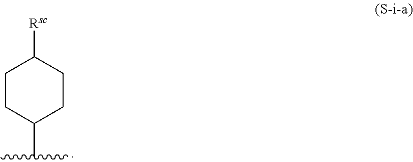

19. The compound of claim 18, wherein R^{sc} is —OR^{SO}.

20. The compound of claim 19, wherein R^{SO} is optionally substituted heterocyclyl.

21. The compound of claim 20, wherein R^{SO} is optionally substituted six-membered heterocyclyl.

22. The compound of claim 21, wherein R^{SO} is substituted tetrahydropyran.

23. The compound of claim 18, wherein R^{sc} is unsubstituted C₁₋₆ alkyl.

24. The compound of claim 23, wherein R^{sc} is methyl.

25. The compound of claim 23, wherein R^{sc} is ethyl.

26. The compound of claim 18, wherein R^{sc} is substituted C₁₋₆ alkyl.

27. The compound of claim 26, wherein
R^{sc} is —CH₂—O—(CH₂)ₛₙ—X^{sc} or —CH₂—O—(CH₂)ₛₘ—O—X^{sd};
sn is 0, 1, 2, 3, 4, 5, or 6;
sm is 1, 2, 3, 4, 5, or 6;
X^{sc} is hydrogen, optionally substituted C₁₋₆ alkyl, optionally substituted aryl, optionally substituted carbocyclyl, or optionally substituted heterocyclyl; and
X^{sd} is optionally substituted C₁₋₆ alkyl, optionally substituted aryl, optionally substituted carbocyclyl, or optionally substituted heterocyclyl.

28. The compound of claim 27, wherein R^{sc} is —CH₂—O—X^{sc}; and X^{sc} is C₁₋₆ haloalkyl.

29. The compound of claim 28, wherein R^{sc} is —CH₂—O—CH₂CF₃.

30. The compound of claim 17, wherein R^{W2} is of Formula (S-i-b):

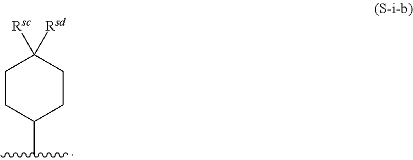

31. The compound of claim 30, wherein each instance of R^{sc} and R^{sd} is independently substituted C₁₋₆ alkyl.

32. The compound of claim 31, wherein each instance of R^{sc} and R^{sd} is independently —C₁₋₆alkyl-OH.

33. The compound of claim 32, wherein R^{sc} and R^{sd} are —CH₂—OH.

34. The compound of claim 31, wherein
each instance of R^{sc} and R^{sd} is independently —CH₂—O—(CH₂)ₛₙ—X^{sc} or —CH₂—O—(CH₂)ₛₘ—O—X^{sd};
sn is 0, 1, 2, 3, 4, 5, or 6;
sm is 1, 2, 3, 4, 5, or 6;
X^{sc} is hydrogen, optionally substituted C₁₋₆ alkyl, optionally substituted aryl, optionally substituted carbocyclyl, or optionally substituted heterocyclyl; and
X^{sd} is optionally substituted C₁₋₆ alkyl, optionally substituted aryl, optionally substituted carbocyclyl, or optionally substituted heterocyclyl.

35. The compound of claim 34, wherein X^{sc} is optionally substituted C₁₋₆ alkyl.

36. The compound of claim 35, wherein X^{sc} is ethyl or n-propyl.

37. The compound of claim 34, wherein X^{sc} is optionally substituted C₃₋₆ carbocyclyl.

38. The compound of claim 37, wherein X^{sc} is cyclopropyl.

39. The compound of claim 34, wherein X^{sc} is optionally substituted heterocyclyl.

40. The compound of claim 39, wherein X^{sc} is tetrahydropyran.

41. The compound of any one of claims 34-40, wherein X^{sd} is optionally substituted alkyl.

42. The compound of claim 41, wherein X^{sd} is substituted alkyl.

43. The compound of claim 41, wherein X^{sd} is unsubstituted alkyl.

44. The compound of claim 43, wherein X^{sd} is ethyl.

45. The compound of claim 17, wherein R^{W2} is of Formula (S-i-c):

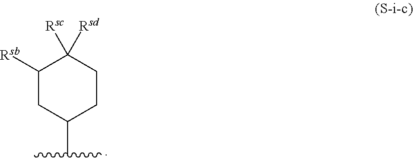

46. The compound of claim 45, wherein each of R^{sc} and R^{sd} is independently unsubstituted C₁₋₆ alkyl; and R^{sb} is optionally substituted alkyl, —C(═O)N(R^{SN1})₂, or —OR^{SO}.

47. The compound of claim 46, wherein R^{sc} and R^{sd} are methyl.

48. The compound of any one of claims 45-47, wherein R^{sb} is independently substituted C₁₋₆ alkyl.

49. The compound of claim 48, wherein:
R^{sb} is —CH₂—O—(CH₂)ₛₙ—X^{sc} or —CH₂—O—(CH₂)ₛₘ—O—X^{sd};
sn is 0, 1, 2, 3, 4, 5, or 6;
sm is 1, 2, 3, 4, 5, or 6;
X^{sc} is hydrogen, optionally substituted C₁₋₆ alkyl, optionally substituted aryl, optionally substituted carbocyclyl, or optionally substituted heterocyclyl; and
X^{sd} is optionally substituted C₁₋₆ alkyl, optionally substituted aryl, optionally substituted carbocyclyl, or optionally substituted heterocyclyl.

50. The compound of claim 49, wherein R^{sb} is —CH₂—O—(CH₂)ₛₙ—X^{sc} and X^{sc} is hydrogen.

51. The compound of claim 50, wherein R^{sb} is —CH₂—OH.

52. The compound of claim 49, wherein R^{sb} is —CH₂—O—(CH₂)ₛₙ—X^{sc} and X^{sc} is optionally substituted C₁₋₆ alkyl.

53. The compound of claim 50, wherein R^{sb} is —CH₂—O—(CH₂)ₛₙ—C₂H₅.

54. The compound of claim 49, wherein R^{sb} is —CH₂—O—(CH₂)ₛₘ—O—X^{sd}; and X^{sd} is unsubstituted alkyl.

55. The compound of claim 54, wherein R^{sb} is —CH₂—O—(CH₂)ₛₘ—O—CH₃.

56. The compound of any one of claims 45-55, wherein R^{sb} is —OR^{SO}; and R^{SO} is optionally substituted alkyl.

57. The compound of claim 56, wherein R^{sb} is —O-isobutyl.

58. The compound of claim 17, wherein R^{sc} and R^{sd} are taken together with the intervening atom to form a substituted heterocyclic ring of Formula (S-i-d): 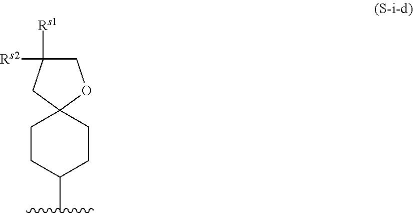
wherein each instance of R^{s1} and R^{s2} is independently optionally substituted C₁₋₆ alkyl.

59. The compound of claim 58, wherein each instance of R^{s1} and R^{s2} is independently unsubstituted C₁₋₆ alkyl.

60. The compound of claim 59, wherein both R^{s1} and R^{s2} are methyl.

61. The compound of claim 17, wherein R^{sc} and R^{sd} are taken together with the intervening atom to form a substituted heterocyclic ring of Formula (S-i-e): 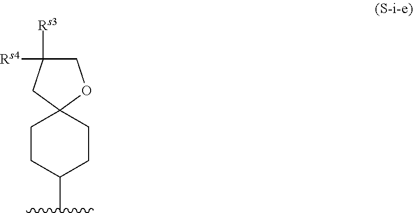
wherein each instance of R^{s3} and R^{s4} is independently optionally substituted C₁₋₆ alkyl.

62. The compound of claim 61, wherein each instance of R^{s3} and R^{s4} is independently unsubstituted C₁₋₆ alkyl.

63. The compound of claim 62, wherein both of R^{s3} and R^{s4} are methyl.

64. The compound of claim 17, wherein R^{sc} and R^{sd} are taken together with the intervening atom to form a substituted heterocyclic ring of Formula (S-i-f1) or (S-i-f2): 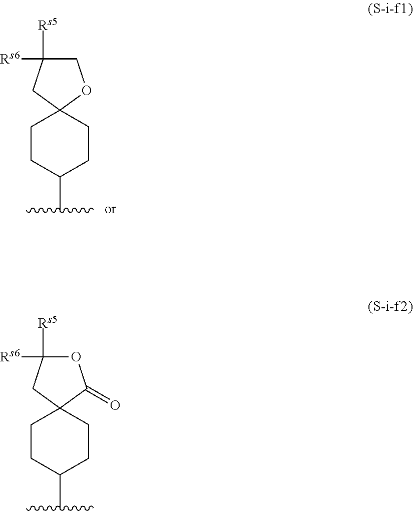
wherein each of R^{s5} and R^{s6} is independently C₁₋₆ alkyl.

65. The compound of claim 64, wherein each of R^{s5} and R^{s6} is independently unsubstituted C₁₋₆ alkyl.

66. The compound of claim 65, wherein R^{s5} and R^{s6} are both methyl.

67. The compound of claim 17, wherein R^{sc} and R^{sd} are taken together with the intervening atom to form a heterocyclic ring of Formula (S-i-g):

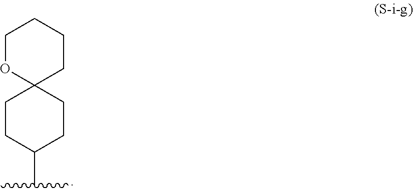 [SMILES: *C1CCC2(CCCCO2)CC1]

68. The compound of claim 17, wherein R^{sc} and R^{sd} are taken together with the intervening atom to form a heterocyclic ring of Formula (S-i-h):

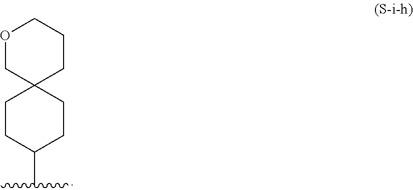 [SMILES: *C1CCC2(CCCOC2)CC1]

69. The compound of claim 17, wherein R^{sc} and R^{sd} are taken together with the intervening atom to form a substituted heterocyclic ring of Formula (S-i-i): 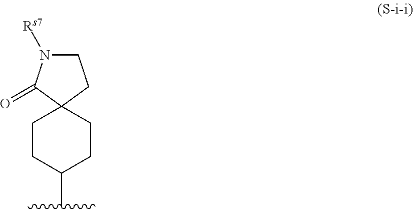
wherein each instance of R^{s7} is optionally substituted alkyl.

70. The compound of claim 69, wherein R^{s7} is optionally substituted C₄₋₈ alkyl.

71. The compound of claim 70, wherein R^{s7} is isopentyl.

72. The compound of claim 17, wherein R^{sc} and R^{sd} are taken together with the intervening atom to form a substituted heterocyclic ring of Formula (S-i-j). 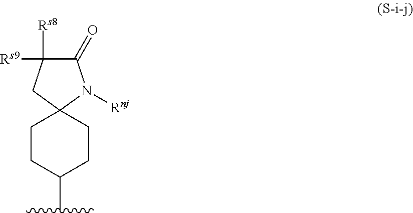
wherein each instance of R^{s8} and R^{s9} is independently hydrogen or optionally substituted C₁₋₆ alkyl; and R^{nj} is independently hydrogen, optionally substituted C₁₋₆ alkyl, or a nitrogen protecting group.

73. The compound of claim 72, wherein R^{s8} and R^{s9} are both methyl or ethyl.

74. The compound of any one of claims 72-73, wherein R^{nj} is hydrogen or methyl.

75. The compound of claim 16, wherein R^{sc} and R^{sd} are taken together with the intervening atom to form a carbocyclic ring of Formula (S-i-l): 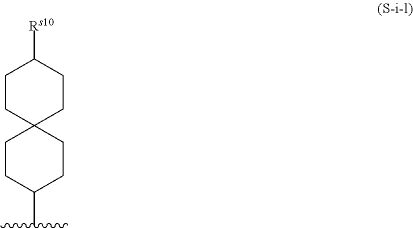
wherein R^{s10} is —OR^{sl}; and R^{sl} is optionally substituted alkyl.

76. The compound of claim 75, wherein R^{sl} is methyl.

77. The compound of claim 17, wherein R^{sa} and R^{sb} are taken together with the intervening atom to form a carbocyclic ring of Formula (S-i-m) 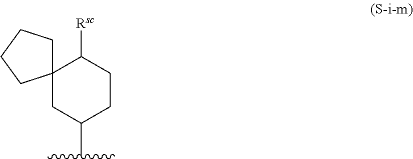
wherein R^{sc} is optionally substituted C₁₋₆ alkyl.

78. The compound of claim 77, wherein R^{sc} is substituted C₁₋₆ alkyl.

79. The compound of claim 78, wherein
R^{sc} is independently —CH₂—O—(CH₂)ₛₙ—X^{sc};
sn is 0, 1, 2, 3, 4, 5, or 6;
X^{sc} is hydrogen, optionally substituted C₁₋₆ alkyl, optionally substituted aryl, optionally substituted carbocyclyl, or optionally substituted heterocyclyl.

80. The compound of claim 79, wherein R^{sc} is —(CH₂)ₛₙ—O—CH₃.

81. The compound of claim 17, wherein R^{W2} is of Formula (S-i-n): 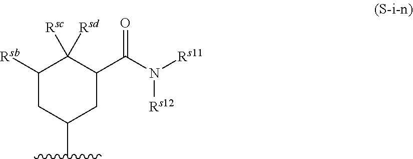
wherein:
each of R^{s11} and R^{s12} is independently hydrogen or optionally substituted C₁₋₆ alkyl; and
R^{sb} is optionally substituted C₁₋₆ alkyl.

82. The compound of claim 81, wherein R^{sb} is ethyl.

83. The compound of claim 17, wherein R^{W2} is of Formula (S-i-o): 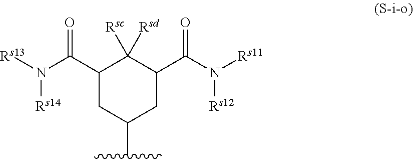
wherein:
each of R^{s13}, R^{s14}, R^{s15}, and R^{s16} is independently hydrogen or optionally substituted C₁₋₆ alkyl.

84. The compound of claim 83, wherein R^{s14} and R^{s16} are both hydrogen.

85. The compound of claim 83, wherein R^{s14} and R^{s16} are both methyl, isobutyl, or isopentyl.

86. The compound of any one of claim 83, wherein R^{s14} is hydrogen and R^{s16} is methyl.

87. The compound of any one of claims 83-86, wherein R^{s13} and R^{s15} are both isobutyl.

88. The compound of any one of claims 83-86, wherein:
each of R^{s13} and R^{s15} is independently —(CH₂)_{sd}—OX^{sd};
sd is 1, 2, 3, 4, or 5; and
X^{sd} is optionally substituted C₁₋₆ alkyl.

89. The compound of claim 88, wherein R^{s13} and R^{s15} are both —(CH₂)₂—OCH₃.

90. The compound of any one of claims 83-86, wherein R^{s13} is methyl or ethyl and R^{s15} is isopentyl.

91. The compound of claim 1, wherein R^{W2} is of Formula (S-ii): 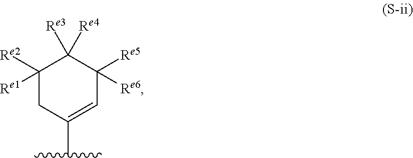
wherein:
each of R^{e1}, R^{e2}, R^{e3}, R^{e4}, R^{e5}, and R^{e6} is independently hydrogen, optionally substituted C₁₋₆ alkyl, or —OR^{eo};
R^{eo} is hydrogen, optionally substituted alkyl, optionally substituted carbocyclyl, optionally substituted heterocyclyl, optionally substituted aryl, optionally substituted heteroaryl, or an oxygen protecting group; and
R^{e1} and R^{e2} are optionally taken with the intervening atom to form an optionally substituted carbocyclic ring; and
R^{e3} and R^{e4} are optionally taken with the intervening atom to form an optionally substituted carbocyclic ring.

92. The compound of claim 91, wherein R^{W2} is of Formula (S-ii-a):

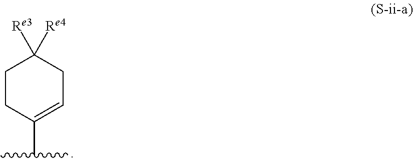

93. The compound of claim 92, wherein each of R^{e3} and R^{e4} is independently optionally substituted C₁₋₆ alkyl.

94. The compound of claim 93, wherein each of R^{e3} and R^{e4} is independently C₁₋₆ haloalkyl.

95. The compound of claim 93, wherein:
each of R^{e3} and R^{e4} is independently hydrogen or —CH₂—O—X^{e1}, and
X^{e1} is optionally substituted C₁₋₆ alkyl.

96. The compound of claim 95, wherein X^{e1} is substituted C₁₋₆ alkyl.

97. The compound of claim 96, wherein X^{e1} is —CH₂—CF₃.

98. The compound of claim 95, wherein X^{e1} is unsubstituted C₁₋₆ alkyl.

99. The compound of claim 98, wherein X^{e1} is ethyl.

100. The compound of claim 92, wherein R^{e3} and R^{e4} are taken with the intervening atom to form an optionally substituted heterocyclyl.

101. The compound of claim 100, wherein R^{W2} is of Formula (S-ii-a1): 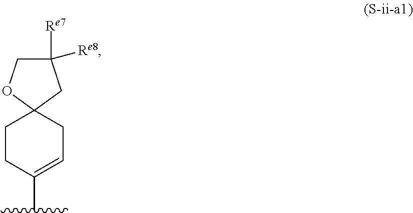
wherein each of R^{e7} and R^{e8} is independently optionally substituted C₁₋₆ alkyl.

102. The compound of claim 101, wherein R^{e7} and R^{e8} are methyl.

103. The compound of claim 91, wherein R^{W2} is of Formula (S-ii-b):

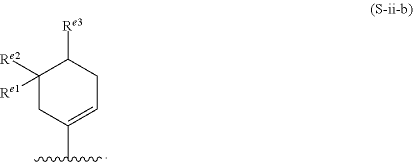

104. The compound of claim 103, wherein R^{e1} and R^{e2} are taken with the intervening atom to form an optionally substituted carbocyclic ring.

105. The compound of claim 104, wherein R^{e1} and R^{e2} are taken with the intervening atom to form an optionally substituted cyclopentyl ring.

106. The compound of any one of claims 103-105, wherein
R^{e3} is —OR^{eo}; and
R^{eo} is optionally substituted C₁₋₆ alkyl.

107. The compound of claim 106, wherein
R^{e3} is —O—(CH₂)_{eb}—O—X^{e2};
X^{e2} is optionally substituted C₁₋₆ alkyl; and
eb is 1, 2, 3, 4, 5, or 6.

108. The compound of claim 107, wherein R^{e3} is —O—(CH₂)₃—O—CH₃.

109. The compound of claim 1, wherein R^{W2} is optionally substituted tetrahydropyran.

110. The compound of claim 109, wherein R^{W2} is of Formula (S-iii): 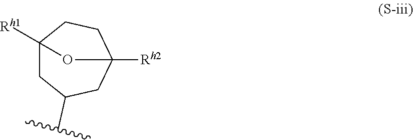
wherein each of R^{h1} and R^{h2} is independently hydrogen, halo, —CN, NO₂, or optionally substituted C₁₋₆ alkyl.

111. The compound of claim 110, wherein R^{h1} is optionally substituted C₁₋₆ alkyl.

112. The compound of claim 111, wherein R^{h1} is unsubstituted C₁₋₆ alkyl.

113. The compound of claim 112, wherein R^{h1} is ethyl.

114. The compound of any one of claims 110-113, wherein R^{h2} is optionally substituted C₁₋₆ alkyl.

115. The compound of claim 114, wherein R^{h2} is unsubstituted C₁₋₆ alkyl.

116. The compound of claim 115, wherein R^{h2} is ethyl.

117. The compound of claim 1, wherein the compound is of Formula (S-I-b): 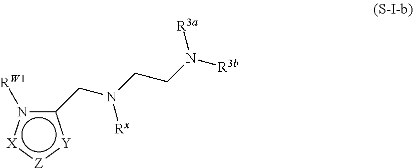
or a pharmaceutically acceptable salt thereof.

118. The compound of claim 117, wherein the compound is of Formula (S-XV): 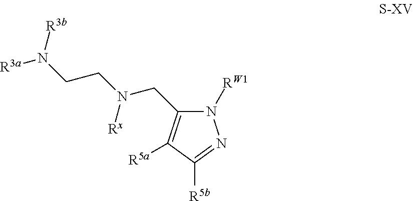
or a pharmaceutically acceptable salt thereof, wherein each of R^{5a} and R^{5b} is independently hydrogen, halo, —CN, NO₂, or optionally substituted C₁₋₄ alkyl.

119. The compound of claim 117, wherein the compound is of Formula (S-XVI): 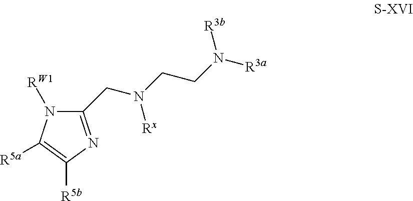
or a pharmaceutically acceptable salt thereof, wherein each of R^{5a} and R^{5b} is independently hydrogen, halo, —CN, NO₂, or optionally substituted C₁₋₆ alkyl.

120. The compound of claim 1, wherein the compound is of Formula (S-XVII): 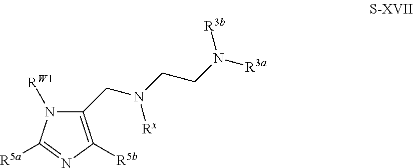
or a pharmaceutically acceptable salt thereof, wherein each of R^{5a} and R^{5b} is independently hydrogen, halo, —CN, NO₂, or optionally substituted C₁₋₆ alkyl.

121. The compound of any one of claims 118-120, wherein R^{5a} is hydrogen.

122. The compound of any one of claims 118-120, wherein R^{5a} is optionally substituted C₁₋₆ alkyl.

123. The compound of claim 122, wherein R^{5a} is unsubstituted C₁₋₆ alkyl.

124. The compound of claim 123, wherein R^{5a} is methyl.

125. The compound of any one of claims 118-120, wherein R^{5a} is halogen.

126. The compound of claim 125, wherein R^{5a} is Cl.

127. The compound of any one of claims 118-126, wherein R^{5b} is hydrogen.

128. The compound of any one of claims 118-126, wherein R^{5b} is optionally substituted C₁₋₆ alkyl.

129. The compound of claim 128, wherein R^{5b} is unsubstituted C₁₋₆ alkyl.

130. The compound of claim 129, wherein R^{5b} is methyl.

131. The compound of any one of claims 118-126, wherein R^{5b} is halogen.

132. The compound of claim 131, wherein R^{5b} is Cl.

133. The compound of claim 1, wherein the compound is of Formula (S-XVIII): 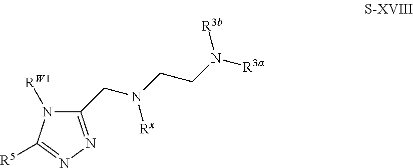
or a pharmaceutically acceptable salt thereof.

134. The compound of claim 1, wherein the compound is of Formula (S-XIX): 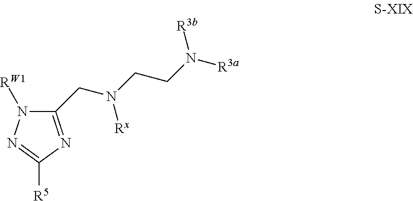
or a pharmaceutically acceptable salt thereof.

135. The compound of claim 1, wherein the compound is of Formula (S-XX): 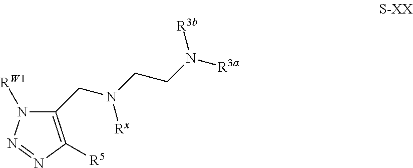
or a pharmaceutically acceptable salt thereof.

136. The compound of any one of claims 117-135, wherein R^{W1} is substituted cyclohexyl of Formula (S-iii): 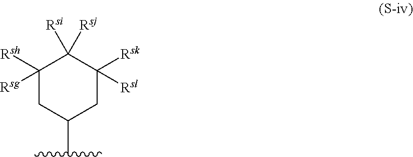
wherein:
each of R^{sg}, R^{sh}, R^{sk}, and R^{sl} is independently hydrogen or optionally substituted C₁₋₆ alkyl,
each of R^{si} and R^{sj} is independently optionally substituted C₁₋₆ alkyl; and
R^{sg} and R^{sh} are optionally taken together with the intervening atom to form an optionally substituted carbocylic ring or an optionally substituted heterocyclic ring;
R^{si} and R^{sj} are optionally taken together with the intervening atom to form an optionally substituted carbocylic ring or an optionally substituted heterocyclic ring; and
R^{sk} and R^{sl} are optionally taken together with the intervening atom to form an optionally substituted carbocylic ring or an optionally substituted heterocyclic ring.

137. The compound of claim 136, wherein R^{si} and R^{sj} are the same.

138. The compound of claim 136, wherein R^{si} and R^{sj} are different.

139. The compound of any one of claims 136-138, wherein each of R^{si} and R^{sj} is independently substituted C₁₋₆ alkyl.

140. The compound of claim 139, wherein each of R^{si} and R^{sj} is independently —CH₂—O—(CH₂)ₛₚ—X^{si}, or —CH₂—O—(CH₂)_{sq}—O—X^{sj};
each instance of sp is 0, 1, 2, 3, 4, 5, or 6;
each instance of sq is 0, 1, 2, 3, 4, 5, or 6; and
each instance of X^{si} and X^{sj} is independently hydrogen, optionally substituted C₁₋₆ alkyl, optionally substituted carbocyclyl, or optionally substituted heterocyclyl.

141. The compound of claim 140, wherein each of R^{si} and R^{sj} is independently —CH₂—O—(CH₂)ₛₚ—X^{si}.

142. The compound of claim 141, wherein X^{si} is hydrogen.

143. The compound of claim 141, wherein X^{si} is optionally substituted C₁₋₆ alkyl.

144. The compound of claim 139, wherein each of R^{si} and R^{sj} is independently —CH₂—O—(CH₂)_{sq}—O—X^{sj}.

145. The compound of claim 144, wherein X^{sj} is hydrogen.

146. The compound of claim 144, wherein X^{sj} is optionally substituted C₁₋₆ alkyl.

147. The compound of claim 136, wherein R^{si} and R^{sj} are taken together with the intervening atom to form an optionally substituted heterocyclic ring of Formula (S-iii-a): 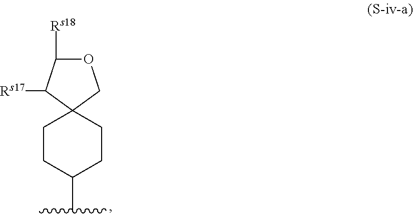
wherein each of R^{s17} and R^{s18} is independently optionally substituted C₁₋₆ alkyl.

148. The compound of claim 147, wherein R^{s17} is hydrogen.

149. The compound of claim 147, wherein R^{s17} is C₁₋₄ alkyl.

150. The compound of claim 147, wherein R^{s17} is methyl.

151. The compound of any one of claims 147-150, wherein R^{s18} is hydrogen.

152. The compound of claim 151, wherein R^{s18} is C₁₋₄ alkyl.

153. The compound of claim 152, wherein R^{s18} is methyl.

154. The compound of claim 136, wherein R^{si} and R^{sj} are taken together with the intervening atom to form a heterocyclic ring of Formula (S-iii-b): 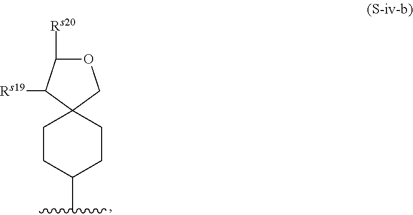
wherein each of R^{s19} and R^{s20} is independently hydrogen or optionally substituted C₁₋₆ alkyl.

155. The compound of claim 154, wherein R^{s19} is hydrogen.

156. The compound of claim 154, wherein R^{s19} is C₁₋₄ alkyl.

157. The compound of claim 156, wherein R^{s19} is methyl.

158. The compound of any one of claims 154-157, wherein R^{s20} is hydrogen.

159. The compound of claim 158, wherein R^{s20} is C₁₋₄ alkyl.

160. The compound of claim 159, wherein R^{s20} is methyl.

161. The compound of any one of claims 1-160, wherein R⁴ is hydrogen.

162. The compound of any one of claims 1-160, wherein R⁴ is optionally substituted C₁₋₆ alkyl.

163. The compound of claim 162, wherein R⁴ is unsubstituted C₁₋₆ alkyl.

164. The compound of claim 163, wherein R⁴ is methyl.

165. The compound of any one of claims 1-164, wherein R⁵ is hydrogen.

166. The compound of any one of claims 1-164, wherein R⁵ is halogen.

167. The compound of claim 166, wherein R⁵ is Cl.

168. The compound of any one of claims 1-164, wherein R⁵ is optionally substituted C₁₋₄ alkyl.

169. The compound of claim 168, wherein R⁵ is unsubstituted C₁₋₄ alkyl.

170. The compound of claim 169, wherein R⁵ is methyl.

171. The compound of any one of claims 1-170, wherein R^{x} is optionally substituted C₁₋₄ alkyl.

172. The compound of claim 171, wherein R^{x} is unsubstituted C₁₋₄ alkyl.

173. The compound of claim 172, wherein R^{x} is methyl.

174. The compound of any one of claims 1-173, wherein R^{3a} is hydrogen.

175. The compound of claim 174, wherein R^{3a} is optionally substituted C₁₋₄ alkyl.

176. The compound of claim 175, wherein R^{3a} is unsubstituted C₁₋₄ alkyl.

177. The compound of claim 176, wherein R^{3a} is methyl.

178. The compound of any one of claims 1-177, wherein R^{3b} is hydrogen.

179. The compound of claim 178, wherein R^{3b} is optionally substituted C₁₋₄ alkyl.

180. The compound of claim 179, wherein R^{3b} is unsubstituted C₁₋₄ alkyl.

181. The compound of claim 180, wherein R^{3b} is methyl.

182. The compound of claim 1, wherein the compound is selected from the group consisting of the compounds depicted in Table 2, and pharmaceutically acceptable salts thereof.

183. A pharmaceutical composition comprising a compound of any one of claims 1-182 or a pharmaceutically acceptable salt thereof, and a pharmaceutically acceptable excipient.

184. A kit or packaged pharmaceutical comprising a compound of any one of claims 1-182 or a pharmaceutically acceptable salt thereof, and instructions for use thereof.

185. A method of inhibiting an arginine methyl tranferase (RMT) comprising contacting a cell with an effective amount of a compound of any one of claims 1-182 or a pharmaceutically acceptable salt thereof.

186. The method of claim 185, wherein the arginine methyl transferase is PRMT1.

187. The method of claim 185, wherein the arginine methyl transferase is PRMT6.

188. The method of claim 185, wherein the arginine methyl transferase is PRMT3.

189. The method of claim 185, wherein the arginine methyl transferase is PRMT8.

190. The method of claim 185, wherein the arginine methyl transferase is CARM1.

191. A method of modulating gene expression comprising contacting a cell with an effective amount of a compound of any one of claims 1-182 or a pharmaceutically acceptable salt thereof.

192. A method of modulating transcription comprising contacting a cell with an effective amount of a compound of any one of claims 1-182 or a pharmaceutically acceptable salt thereof.

193. The method of any one of claims 185-192, wherein the cell is in vitro.

194. The method of any one of claims 185-192, wherein the cell is in a subject.

195. A method of treating a RMT-mediated disorder, comprising administering to a subject in need thereof a therapeutically effective amount of a compound of any one of claims 1-182, or a pharmaceutically acceptable salt thereof, or a pharmaceutical composition of claim 183.

196. The method of claim 195, wherein the RMT-mediated disorder is a PRMT1-mediated disorder.

197. The method of claim 195, wherein the RMT-mediated disorder is a PRMT6-mediated disorder.

198. The method of claim 195, wherein the RMT-mediated disorder is a PRMT3-mediated disorder.

199. The method of claim 195, wherein the RMT-mediated disorder is a PRMT8-mediated disorder.

200. The method of claim 195, wherein the RMT-mediated disorder is a CARM1-mediated disorder.

201. The method of claim 195, wherein the disorder is a proliferative disorder.

202. The method of claim 195, wherein the disorder is cancer.

203. The method of claim 195, wherein the disorder is a neurological disorder.

204. The method of claim 203, wherein the disorder is amyotrophic lateral sclerosis.

205. The method of claim 195, wherein the disorder is a muscular dystrophy.

206. The method of claim 195, wherein the disorder is an autoimmune disorder.

207. The method of claim 195, wherein the disorder is a vascular disorder.

208. The method of claim 195, wherein the disorder is a metabolic disorder.


SEQUENCE LISTING (17 sequences)

SEQ ID NO: 1 - protein, 15 aa, Artificial Sequence
  Synthetic Polypeptide
RLARRGGVKRISGLI

SEQ ID NO: 2 - protein, 8 aa, Artificial Sequence
  Synthetic Polypeptide
DYKDDDDK

SEQ ID NO: 3 - protein, 230 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGGNS

SEQ ID NO: 4 - protein, 609 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGGNSDYKDDDDKMAAAEAANCIMENFVATLANGMSLQPPLEEVSCGQAESSEKPNAEDMTSKDYYFDSYAHFGIHEEMLKDEVRTLTYRNSMFHNRHLFKDKVVLDVGSGTGILCMFAAKAGARKVIGIECSSISDYAVKIVKANKLDHVVTIIKGKVEEVELPVEKVDIIISEWMGYCLFYESMLNTVLYARDKWLAPDGLIFPDRATLYVTAIEDRQYKDYKIHWWENVYGFDMSCIKDVAIKEPLVDVVDPKQLVTNACLIKEVDIYTVKVEDLTFTSPFCLQVKRNDYVHALVAYFNIEFTRCHKRTGFSTSPESPYTHWKQTVFYMEDYLTVKTGEEIFGTIGMRPNAKNNRDLDFTIDLDFKGQLCELSCSTDYRMR

SEQ ID NO: 5 - protein, 15 aa, Artificial Sequence
  Synthetic Polypeptide
RLARRGGVKRISGLI

SEQ ID NO: 6 - protein, 9 aa, Artificial Sequence
  Synthetic Polypeptide
MDYKDDDDK

SEQ ID NO: 7 - protein, 389 aa, Artificial Sequence
  Synthetic Polypeptide
MDYKDDDDKSQPKKRKLESGGGGEGGEGTEEEDGAEREAALERPRRTKRERDQLYYECYSDVSVHEEMIADRVRTDAYRLGILRNWAALRGKTVLDVGAGTGILSIFCAQAGARRVYAVEASAIWQQAREVVRFNGLEDRVHVLPGPVETVELPEQVDAIVSEWMGYGLLHESMLSSVLHARTKWLKEGGLLLPASAELFIAPISDQMLEWRLGFWSQVKQHYGVDMSCLEGFATRCLMGHSEIVVQGLSGEDVLARPQRFAQLELSRAGLEQELEAGVGGRFRCSCYGSAPMHGFAIWFQVTFPGGESEKPLVLSTSPFHPATHWKQALLYLNEPVQVEQDTDVSGEITLLPSRDNPRRLRVLLRYKVGDQEEKTKDFAMEDHHHHHH

SEQ ID NO: 8 - protein, 15 aa, Artificial Sequence
  Synthetic Polypeptide
KPAIRRLARRGGVKR

SEQ ID NO: 9 - protein, 229 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEF

SEQ ID NO: 10 - protein, 623 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFMGMKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 11 - protein, 19 aa, Artificial Sequence
  Synthetic Polypeptide
GGRGGFGGRGGFGGRGGFG

SEQ ID NO: 12 - protein, 226 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGS

SEQ ID NO: 13 - protein, 756 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSCSLASGATGGRGAVENEEDLPELSDSGDEAAWEDEDDADLPHGKQQTPCLFCNRLFTSAEETFSHCKSEHQFNIDSMVHKHGLEFYGYIKLINFIRLKNPTVEYMNSIYNPVPWEKEEYLKPVLEDDLLLQFDVEDLYEPVSVPFSYPNGLSENTSVVEKLKHMEARALSAEAALARAREDLQKMKQFAQDFVMHTDVRTCSSSTSVIADLQEDEDGVYFSSYGHYGIHEEMLKDKIRTESYRDFIYQNPHIFKDKVVLDVGCGTGILSMFAAKAGAKKVLGVDQSEILYQAMDIIRLNKLEDTITLIKGKIEEVHLPVEKVDVIISEWMGYFLLFESMLDSVLYAKNKYLAKGGSVYPDICTISLVAVSDVNKHADRIAFWDDVYGFKMSCMKKAVIPEAVVEVLDPKTLISEPCGIKHIDCHTTSISDLEFSSDFTLKITRTSMCTAIAGYFDIYFEKNCHNRVVFSTGPQSTKTHWKQTVFLLEKPFSVKAGEALKGKVTVHKNKKDPRSLTVTLTLNNSTQTYGLQ

SEQ ID NO: 14 - protein, 15 aa, Artificial Sequence
  Synthetic Polypeptide
PRKQLATKAARKSAP

SEQ ID NO: 15 - protein, 8 aa, Artificial Sequence
  Synthetic Polypeptide
EGHHHHHH

SEQ ID NO: 16 - protein, 601 aa, Artificial Sequence
  Synthetic Polypeptide
MDYKDDDDKAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLGSSGAQGSGGGSTSAHYAVNSQFTMGGPAISMASPMSIPTNTMHYGSEGHHHHHH

SEQ ID NO: 17 - protein, 6 aa, Artificial Sequence
  Synthetic Polypeptide
HHHHHH